(19) United States
(12) Patent Application Publication
(10) Pub. No.: US 2026/0069718 A1
(43) Pub. Date: Mar. 12, 2026

(54) Engineered Guide RNAs and Polynucleotides

(71) Applicant: Shape Therapeutics Inc., Seattle, WA (US)

(72) Inventors: Yiannis SAVVA, Seattle, WA (US); Lina Rajili BAGEPALLI, Cambridge, MA (US); Brian BOOTH, West Roxbury, MA (US); Adrian Wrangham BRIGGS, Seattle, WA (US); Richard Thomas SULLIVAN, Seattle, WA (US); Yue JIANG, Los Angeles, CA (US)

(21) Appl. No.: 19/243,831

(22) Filed: Jun. 20, 2025

Related U.S. Application Data
(63) Continuation of application No. PCT/US2023/085440, filed on Dec. 21, 2023.
(60) Provisional application No. 63/434,986, filed on Dec. 23, 2022.
(60) Provisional application No. 63/528,027, filed on Jul. 20, 2023.

(51) Int. Cl. A61K 48/00; C12N 9/78; C12N 15/11; C12N 15/86

(52) U.S. Cl. CPC A61K 48/0066; C12N 9/78; C12N 15/11; C12N 15/86; C12N 2310/20; C12N 2320/33; C12N 2750/14143

(57) ABSTRACT

Disclosed herein are engineered guide RNAs and compositions comprising the same for treatment of diseases or conditions in a subject. Also disclosed herein are methods of treating diseases or conditions in a subject by administering engineered guide RNAs or pharmaceutical compositions described herein.

### CROSS-REFERENCE TO RELATED APPLICATIONS

This application is a continuation of PCT/US2023/085440, filed Dec. 21, 2023, which claims priority under 35 U.S.C. § 119 from Provisional Application Ser. No. 63/528,027 filed on Jul. 20, 2023, and Provisional Application Ser. No. 63/434,986 filed on Dec. 23, 2022, the disclosures of which are incorporated herein by reference.

### SEQUENCE LISTING

The instant application contains a Sequence Listing which has been submitted electronically in ST.26 (xml) format and is hereby incorporated by reference in its entirety. Said ST.26 (xml) copy, created on Jan. 11, 2024, is named 199235-758601_xml and is 774,485 bytes in size.

### BACKGROUND

Compositions that mediate RNA editing can be viable therapies for genetic diseases. However, highly efficacious compositions that can maximize on-target RNA editing while minimizing off-target RNA editing are needed. Moreover, compositions that are capable of facilitating RNA editing are also needed.

### SUMMARY

Disclosed herein is a composition comprising an engineered guide RNA or a polynucleotide encoding the engineered guide RNA, wherein the engineered guide RNA has complementarity to a target sequence of a target SNCA RNA and comprises a polynucleotide sequence having at least about: 80%, 85%, 90%, 92%, 95%, 97%, or 99% sequence identity to any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784; wherein upon hybridization of the engineered guide RNA to the target sequence of the target SNCA RNA, a guide-target RNA scaffold is formed that has one or more structural features that is not present within the engineered guide RNA prior to the hybridization and is selected from the group consisting of: a bulge, an internal loop, and a hairpin; and wherein formation of the guide-target RNA scaffold results in knockdown of alpha-synuclein protein encoded by the target SNCA RNA. In some embodiments, the engineered guide RNA comprises a polynucleotide sequence having at least about: 80%, 85%, 90%, 92%, 95%, 97%, or 99% sequence identity to any one of SEQ ID NOS: 293-299 or SEQ ID NOS: 333-340. In some embodiments, the engineered guide RNA comprises the polynucleotide sequence of any one of SEQ ID NOS: 293-299 or SEQ ID NOS: 333-340. In some embodiments, the target sequence of the target SNCA RNA comprises a translation initiation site. In some embodiments, the translation initiation site is the SNCA Codon 1 translation initiation site of Exon 2. In some embodiments, the translation initiation site is the SNCA Codon 1 translation initiation site of Exon 2 corresponding to position 226 of an SNCA transcript variant 1 of accession number NM_000345.4. In some embodiments, the one or more structural features comprises at least a first 6/6 symmetric internal loop and at least a second 6/6 symmetric loop. In some embodiments, the first 6/6 symmetric internal loop is at a position selected from the group consisting of: 33, 32, 30, 28, and 26; relative to a target adenosine at position 0. In some embodiments, the first 6/6 symmetric internal loop is at position 33, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, or 99% sequence identity to SEQ ID NO: 337. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, or 99% sequence identity to SEQ ID NO: 338 or SEQ ID NO: 339. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, an A/C mismatch at position 0, a G/U wobble base at position 2 relative to position 0, a U/G wobble base at position 5 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, or 99% sequence identity to SEQ ID NO: 340. In some embodiments, the one or more structural features further comprises at least one structural feature selected from the group consisting of: the second 6/6 symmetric internal loop at position −6 relative to position 0, an A/C mismatch at position 0, and any combination thereof. In some embodiments, the engineered guide RNA comprises at least about: 80%, 85%, 90%, 92%, 95%, 97%, or 99% sequence identity to SEQ ID NO: 298 or SEQ ID NO: 299. In some embodiments, the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof. In some embodiments, the engineered guide RNA comprises at least about: 80%, 85%, 90%, 92%, 95%, 97%, or 99% sequence identity to SEQ ID NO: 299. In some embodiments, the first 6/6 symmetric internal loop is at position 32, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, or 99% sequence identity to SEQ ID NO: 333. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, or 99% sequence identity to SEQ ID NO: 334. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, or 99% sequence identity to SEQ ID NO: 335. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, a G/U wobble base at position −3 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, or 99% sequence identity to SEQ ID NO: 336. In some embodiments, the one or more structural features further comprises at least one structural feature selected from the group consisting of: the second 6/6 symmetric internal loop at position −6 relative to position 0, an A/C mismatch at position 0, and any combination thereof. In some embodiments, the engineered guide RNA comprises at least about: 80%, 85%, 90%, 92%, 95%, 97%, or 99% sequence identity to SEQ ID NO: 295. In some embodiments, the first 6/6 symmetric internal loop is at position 30, relative to the target adenosine at position 0. In some embodiments, the one or more structural features further comprises at least one structural feature selected from the group consisting of: the second 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof. In some embodiments, the engineered guide RNA comprises at least about: 80%, 85%, 90%, 92%, 95%, 97%, or 99% sequence identity to SEQ ID NO: 297. In some embodiments, the first 6/6 symmetric internal loop is at position 28, relative to the target adenosine at position 0. In some embodiments, the one or more structural features further comprises at least one structural feature selected from the group consisting of: the second 6/6 symmetric internal loop at position −8 relative to position 0, an A/C mismatch at position 0, a G/U wobble base pair at position 2 relative to position 0, and any combination thereof. In some embodiments, the engineered guide RNA comprises at least about: 80%, 85%, 90%, 92%, 95%, 97%, or 99% sequence identity to SEQ ID NO: 293. In some embodiments, the one or more structural features further comprises at least one structural feature selected from the group consisting of: the second 6/6 symmetric internal loop at position −10 relative to position 0, a 0/1 asymmetric bulge at position −6 relative to position 0, an A/C mismatch at position 0, an A/A mismatch at position 4 relative to position 0, and any combination thereof. In some embodiments, the engineered guide RNA comprises at least about: 80%, 85%, 90%, 92%, 95%, 97%, or 99% sequence identity to SEQ ID NO: 294. In some embodiments, the first 6/6 symmetric internal loop is at position 26, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof. In some embodiments, the engineered guide RNA comprises at least about: 80%, 85%, 90%, 92%, 95%, 97%, or 99% sequence identity to SEQ ID NO: 296. In some embodiments, the one or more structural features comprises the bulge, and wherein the bulge is a symmetric bulge. In some embodiments, the one or more structural features comprises the bulge, and wherein the bulge is an asymmetric bulge. In some embodiments, the one or more structural features comprises the internal loop, and wherein the internal loop is a symmetric internal loop. In some embodiments, the one or more structural features comprises the internal loop, and wherein the internal loop is an asymmetric internal loop. In some embodiments, the guide-target RNA scaffold comprises a wobble base pair. In some embodiments, the one or more structural features comprises the hairpin, and wherein the hairpin is a recruitment hairpin or a non-recruitment hairpin. In some embodiments, upon hybridization of the engineered guide RNA to the target sequence of the target SNCA RNA, the engineered guide RNA facilitates RNA editing of one or more adenosines in the target sequence of the target SNCA RNA by an RNA editing entity. In some embodiments, the RNA editing entity comprises ADAR1, ADAR2, ADAR3, or any combination thereof. In some embodiments, the composition comprises the engineered polynucleotide encoding the engineered guide RNA. In some embodiments, the engineered polynucleotide is comprised in or on a vector. In some embodiments, the vector is a viral vector, and wherein the engineered polynucleotide is encapsidated in the viral vector. In some embodiments, the viral vector is an adeno-associated viral (AAV) vector, a derivative thereof. In some embodiments, the viral vector is an adeno-associated viral (AAV) and wherein the AAV vector is an AAV1 vector, AAV2 vector, AAV3 vector, AAV4 vector, AAV5 vector, AAV6 vector, AAV7 vector, AAV8 vector, AAV9 vector, AAV10 vector, AAV11 vector, or a derivative, a chimera, or a variant of any of these. In some embodiments, the AAV vector is a recombinant AAV (rAAV) vector, a hybrid AAV vector, a chimeric AAV vector, a self-complementary AAV (scAAV) vector, or any combination thereof. In some embodiments, the target sequence of the target SNCA RNA has a polynucleotide sequence that is at least 80% identical to SEQ ID NO: 300. In some embodiments, the engineered guide RNA comprises the sequence of any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784.

Also disclosed herein is a composition that comprises an engineered guide RNA or a polynucleotide encoding the engineered guide RNA, wherein the engineered guide RNA has a length of from 85 nucleotides to 100 nucleotides and hybridizes to at least 80 bases of a target RNA sequence, and the target RNA sequence has at least 80%, at least 85%, at least 90%, at least 92%, at least 95%, at least 96%, at least 97%, at least 98%, at least 99%, or 100% sequence identity to SEQ ID NO: 300. In some embodiments, upon hybridization of the engineered guide RNA to the at least 80 bases of the target RNA sequence, the engineered guide RNA facilitates RNA editing of one or more adenosines in the at least 80 bases of the target RNA sequence by an RNA editing entity. In some embodiments, the engineered guide RNA, upon hybridization to the at least 80 bases of the target RNA sequence, forms a guide-target RNA scaffold that comprises one or more structural features. In some embodiments, the one or more structural features comprises a bulge, wherein the bulge is a symmetric bulge. In some embodiments, the one or more structural features comprises a bulge, wherein the bulge is an asymmetric bulge. In some embodiments, the one or more structural features comprises an internal loop, wherein the internal loop is a symmetric internal loop. In some embodiments, the one or more structural features comprises an internal loop, wherein the internal loop is an asymmetric internal loop. In some embodiments, the one or more structural features comprises a Wobble base pair. In some embodiments, the one or more structural features comprises a hairpin, wherein the hairpin is a recruitment hairpin or a non-recruitment hairpin. In some embodiments, the RNA editing entity comprises ADAR1, ADAR2, ADAR3, or any combination thereof. In some embodiments, the composition comprises an engineered polynucleotide encoding the engineered guide RNA. In some embodiments, the engineered polynucleotide encoding the engineered guide RNA is comprised in or on a vector. In some embodiments, the vector is a viral vector, and wherein the engineered polynucleotide encoding the engineered guide RNA is encapsidated in the viral vector. In some embodiments, the viral vector is an adeno-associated viral (AAV) vector or a derivative thereof. In some embodiments, the AAV vector is an AAV1 vector, AAV2 vector, AAV3 vector, AAV4 vector, AAV5 vector, AAV6 vector, AAV7 vector, AAV8 vector, AAV9 vector, AAV10 vector, AAV11 vector, or a derivative, a chimera, or a variant thereof. In some embodiments, the AAV vector is a recombinant AAV (rAAV) vector, a hybrid AAV vector, a chimeric AAV vector, a self-complementary AAV (scAAV) vector, or any combination thereof.

Also disclosed herein is a pharmaceutical composition comprising: (a) a composition as described herein; and (b) a pharmaceutically acceptable: excipient, carrier, or diluent.

Also disclosed herein is a method of treating a disease or a condition in a subject in need thereof, the method comprising: administering to the subject a therapeutically effective amount of a composition as described herein, or a pharmaceutical composition as described herein. In some embodiments, the disease or condition comprises a synucleinopathy. In some embodiments, the synucleinopathy comprises a Parkinson's disease. In some embodiments, the subject is a human or a non-human animal. In some embodiments, the pharmaceutical composition or the composition is in unit dose form. In some embodiments, the administering is sufficient to treat one or more symptoms of the disease of condition. In some embodiments, the disease or condition is a synucleinopathy. In some embodiments, the one of more symptoms treated comprises muscle tone rigidity, bradykinesia, resting tremor, or any combination thereof. In some embodiments, the administering is sufficient to reduce aggregation of alpha-synuclein protein, relative to: (a) a level of aggregation prior to the administering; (b) a level of accumulated aggregation in the subject in the absence of the administering; or (c) both.

Also disclosed herein is a method of treating a Parkinson's disease in a subject in need thereof, the method comprising: administering to the subject a composition as described herein, or a pharmaceutical composition as described herein, in an amount sufficient to treat the Parkinson's disease in the subject. In some embodiments, the administering is sufficient to treat one or more symptoms of the Parkinson's disease in the subject, relative to prior to the administering. In some embodiments, the one of more symptoms treated comprises muscle tone rigidity, bradykinesia, resting tremor, or any combination thereof. In some embodiments, the subject after the administering displays an increased Unified Parkinson's Disease Rating Scale (UPDRS) score, relative to a UPDRS score prior to the administering.

Also disclosed herein is a method of reducing expression of an alpha-synuclein protein in a subject in need thereof, the method comprising administering the subject a composition as described herein; wherein the administering is sufficient to reduce expression of alpha-synuclein protein in the subject, relative to an amount of alpha-synuclein protein prior to the administering, as determined by an in-vitro assay, thereby reducing the expression of the alpha-synuclein protein in the subject. In some embodiments, the engineered guide RNA has complementarity to a target sequence of a target SNCA RNA that is sufficient for the engineered guide RNA to hybridize to the target sequence of the target SNCA RNA, wherein the target sequence comprises a translation initiation site in the target SNCA RNA. In some embodiments, the translation initiation site is an SNCA Codon 1 translation initiation site of Exon 2. In some embodiments, the target SNCA RNA comprises a pre-mRNA transcript of SNCA. In some embodiments, hybridization of the engineered guide RNA to the target sequence of the target SNCA RNA results in editing of one or more adenosines in the target sequence by an RNA editing entity present in the subject. In some embodiments, at least 40%, at least 50%, at least 60%, at least 70%, at least 80%, at least 85%, at least 90%, at least 95%, or at least 99% of the pre-mRNA transcripts of SNCA have the edit of the one or more adenosines in the target sequence. In some embodiments, the editing of the one or more adenosines in the target sequence of the target SNCA RNA facilitates the reduced expression of alpha-synuclein protein in the subject. In some embodiments, the reduced expression of alpha-synuclein protein is a reduction of at least 10%, relative to an amount of alpha-synuclein protein present prior to the administering. In some embodiments, the reduced expression of alpha-synuclein protein is a reduction of at least 20%, relative to an amount of alpha-synuclein protein present prior to the administering. In some embodiments, the reduced expression of alpha-synuclein protein is a reduction of at least 25%, relative to an amount of alpha-synuclein protein present prior to the administering. In some embodiments, the reduced expression of alpha-synuclein protein is a reduction of at least 30%, relative to an amount of alpha-synuclein protein present prior to the administering. In some embodiments, the reduced expression of alpha-synuclein protein is a reduction of at least 35%, relative to an amount of alpha-synuclein protein present prior to the administering. In some embodiments, the reduced expression of alpha-synuclein protein is a reduction of at least 30%, relative to an amount of alpha-synuclein protein present prior to the administering. In some embodiments, the reduced expression of alpha-synuclein protein is a reduction of at least 40%, relative to an amount of alpha-synuclein protein present prior to the administering. In some embodiments, the reduced expression of alpha-synuclein protein is a reduction of at least 45%, relative to an amount of alpha-synuclein protein present prior to the administering. In some embodiments, the reduced expression of alpha-synuclein protein is a reduction of at least 50%, relative to an amount of alpha-synuclein protein present prior to the administering. In some embodiments, the reduced expression of alpha-synuclein protein is a reduction of from about 10% up to 20%, relative to an amount of alpha-synuclein protein present prior to the administering. In some embodiments, the reduced expression of alpha-synuclein protein is a reduction of from about 20% up to about 30%, relative to an amount of alpha-synuclein protein present prior to the administering. In some embodiments, the reduced expression of alpha-synuclein protein is a reduction of from about 30% up to about 40%, relative to an amount of alpha-synuclein protein present prior to the administering. In some embodiments, the reduced expression of alpha-synuclein protein is a reduction of from about 40% up to about 50%, relative to an amount of alpha-synuclein protein present prior to the administering. In some embodiments, the reduced expression of alpha-synuclein protein is a reduction of from about 50% up to about 60%, relative to an amount of alpha-synuclein protein present prior to the administering. In some embodiments, the reduced expression of alpha-synuclein protein is a reduction of from about 60% up to about 70%, relative to an amount of alpha-synuclein protein present prior to the administering. In some embodiments, the target sequence of the target SNCA RNA comprises a sequence that is at least 80% identical to SEQ ID NO: 300. In some embodiments, hybridization of the engineered guide RNA to the target sequence of the target SNCA RNA results in exon skipping in the pre-mRNA transcript of SNCA. In some embodiments, the exon skipping generates an SNCA mRNA alternate splice variant that does not comprise exon 2 of a wildtype SNCA mRNA transcript. In some embodiments, the exon skipping results in a reduction in alpha-synuclein protein. In some embodiments, the reducing expression of alpha-synuclein protein in the subject comprises a decrease in alpha-synuclein protein level in a biological sample from the subject relative to: (i) an alpha-synuclein protein level in a biological sample obtained from the subject prior to the administering; or (ii) a reference alpha-synuclein protein level obtained from a subject that has a Parkinson's disease, as determined by an in-vitro assay. In some embodiments, the method further comprises reducing a level of alpha-synuclein RNA transcripts comprising exon 2 in the subject relative to: (i) a level of alpha-synuclein RNA transcripts comprising Exon 2 in a biological sample obtained from the subject prior to the administering; or (ii) a reference a level of alpha-synuclein RNA transcripts comprising exon 2 obtained from a subject that has a Parkinson's disease, as determined by an in-vitro assay. In some embodiments, the method treats a disease or condition in the subject. In some embodiments, the disease or condition is at least one selected from the group consisting of: a neurodegenerative disease, a Parkinson's Disease, tremors, muscle stiffness, muscle rigidity, bradykinesia, Dementia with Lewy Bodies (DLB), Multiple System Atrophy (MSA), Pure Autonomic Failure (PAF) and REM sleep Behavior Disorder (RBD).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side).

FIG. 2 shows a schematic of the alpha-synuclein (SNCA, α-Syn) target indicating the translation initiation site (TIS) in Exon 2 of the RNA transcript.

FIG. 3 shows a graph of α-Syn protein expression quantification relative to beta actin controls from a western blot. WT-SH-SY5Y cells are shown in gray bars with circles. TIS c.1 A>G hardwired mutation is shown at 0. TIS c.5 A>G hardwired mutation does not impact α-syn protein expression as shown in black squares. 3′UTR A>G hardwired mutation does not impact α-syn protein expression as shown in white bars with triangles. Y-Axis: Total α-Syn/β-actin, X-axis: SH-SY5Y Cells Undifferentiated or SH-SY5Y Cells Differentiated.

FIGS. 4A-4B show the selection of guide RNA architectures to target SNCA. FIG. 4A shows a graph of the percent RNA editing by gRNAs from a high-throughput screen in the presence of ADAR1 or ADAR1+ADAR2 relative to control RNA. Y-axis: Percent RNA editing; X-axis: Guide (numbered 1-50) C: control. FIG. 4B shows the selected guide RNAs for further optimization with diverse secondary structures.

FIGS. 5A-5C show on-target and bystander editing results for each of the selected SNCA-targeting guides. FIG. 5A shows a schematic of RNA editing for unedited transcripts, on-target edits, on and off target edits, upstream off-target edits, and downstream target edits. FIG. 5B shows the percentage of edits in the presence of ADAR1. FIG. 5C shows the percentage of edits in the presence of both ADAR1 and ADAR2.

FIG. 6 shows a graph of the on-target editing fraction for engineered guide RNAs based on the parental guide RNAs having a polynucleotide sequence of SEQ ID NO: 298, SEQ ID NO: 375, or SEQ ID NO: 784. Editing fraction increases with guide length. Y-axis: On-target editing fraction; X-axis: shows the macrofootprints denoted as length of the guide.mismatch position which includes 80.40, 80.45, 90.50, 95.55, and 100.60.

FIGS. 7A-7B shows barbell scanning results. FIG. 7A shows a schematic of barbell scanning relative to the targeting site of the guide RNA comprising a mismatched nucleobase. FIG. 7B shows heatmaps indicating positions for left barbell and right barbell of exemplary guide RNAs based on the parental guide RNAs having a polynucleotide sequence of SEQ ID NO: 298, SEQ ID NO: 375, or SEQ ID NO: 784 that have the highest RNA editing capability.

FIGS. 8A-8B depict macrofootprint optimization of exemplary guide RNAs using a transient transfection assay. FIG. 8A shows the effect of guide RNA length on editing efficiency for select guide RNAs via ADAR. FIG. 8B depicts editing the editing profile of exemplary guides as a function of nucleotide position.

FIGS. 9A-9B depict SNCA RNA editing in mouse primary neurons with treated with select gRNAs. FIG. 9A shows a graph of relative SNCA protein levels in following delivery of gRNA1 or gRNA2 to mouse primary neurons relative to control RNA. Y-axis: Relative SNCA protein level; X-axis: Condition; regulatory element variants reg1, reg2, reg3, and reg4. FIG. 9B shows RNA editing in response to gRNA1 or gRNA2 relative to control RNA. Y-axis: Percent SNCA TIS editing; X-axis: Condition; regulatory element variants reg1, reg2, reg3, and reg4. Error bars represent standard deviation.

FIGS. 10A-10B show that selected SNCA-targeting gRNAs result in targeted alpha-synuclein protein knockdown and RNA editing in-vivo. FIG. 10A shows micrograms (μg) of human SNCA (hSNCA) per milligram (mg) of total protein in mouse brains administered gRNAs. Y-axis: μg hSNCA/mg total protein; X-axis: Condition; regulatory element variants reg1, reg2, reg3, and reg4. Stats: One-Way ANOVA with Dunnet post-hoc p<0.005, *p<0.05 compared to control gRNA FIG. 10B shows RNA editing in response to gRNA1 or gRNA2 relative to control RNA administered in-vivo. Y-axis: Percent SNCA TIS editing; X-axis: Condition; regulatory element variants reg1, reg2, reg3, and reg4. Mice were administered AAV-gRNA at a dose of 1.55e11 vg/mouse via bilateral ICV. Y-axis: μg hSNCA/mg total protein; X-axis: Condition. N=8 mice. Stats: One-Way ANOVA with Dunnett post-hoc ** p<0.005, *p<0.05 compared to control gRNA.

FIGS. 11A-FIG. 11B shows alternative splice variants detected in mouse models and in-vitro in primary neurons. FIG. 11A shows a schematic of a splice variant detected in SNCA. FIG. 11B shows a gel of RT-PCR products from primary neuron and brain samples treated with SNCA-targeting gRNAs. Bands are indicated as showing exon 2 with arrows.

FIG. 12A-12C show relationships between gRNA architecture and exon skipping in SNCA transcripts in mouse models and in-vitro in primary neurons. FIG. 12A shows a graph of the approximate % of novel splice variants (NSV) for the SNCA-targeting guide RNAs relative to control gRNAs in primary neurons (densitometry). Y-axis: % of novel splice variants (NSV); X-axis: Conditions; Reg: regulatory element. FIG. 12B shows a graph of the approximate % of novel splice variants (NSV) for the SNCA-targeting guide RNAs relative to control gRNAs in mouse brain following intracerebral injection (densitometry). Y-axis: % of novel splice variants (NSV); X-axis: Conditions; regulatory element variants reg1 and reg3. FIG. 12C shows a graph of percent transcript with exon 1-3 splice junction for control, LCOR gRNA, gRNA1, and gRNA2. Y-axis: % of transcripts with 1-3 splice junctions; X-axis: Conditions. SNCA transcripts were measured by droplet digital PCR (ddPCR).

FIG. 13 shows viral genome and gRNA quantification in mice treated with SNCA-targeting gRNAs. FIG. 13 shows a graph of viral genomes per diploid genome (VG/DG) isolated from mouse brains treated with selected gRNAs. Y-axis: VG/DG; X-Axis: Conditions; N=8/group. regulatory element variants reg1 and reg3. Stats: One-Way ANOVA with Dunnett post-hoc **p<0.005, *p<0.05 compared to control gRNA.

FIGS. 14A-14C show α-Syn protein knockdown in human neural stem cell (NSC)-derived dopaminergic neurons treated with select SNCA-targeting gRNAs. FIG. 14A shows images of variable NSC-derived dopaminergic neuronal cultures treated with SNCA gRNAs. Top image shows a population of primarily neuronal cells. Bottom image shows a mixed cell population. FIG. 14B shows a graph of α-Syn protein levels relative to non-transduced control cells. Y-axis: Relative α-Syn protein levels normalized to non-transduced control cells. + indicates presence of two hnRNP A1 within the vector; − indicates the absence of hnRNP A1 in the vector. X-axis: Conditions. 2 replicates per condition were tested. FIG. 14C shows a graph of the percentage of TIS editing in human NSC-derived dopaminergic neurons treated with SNCA gRNAs. + indicates presence of two hnRNP A1 within the vector; − indicates the absence of hnRNP A1 in the vector. X-axis: Conditions. 2 replicates per condition were tested.

FIGS. 15A-15B show α-Syn protein knockdown in human neural stem cell (NSC)-derived dopaminergic neurons treated with select SNCA-targeting gRNAs. FIG. 15A shows a graph of the percentage of TIS editing in human neurons treated with gRNA1, gRNA2, gRNA, and controls. Y-axis: Percent SNCA TIS editing; + indicates presence of two hnRNP A1 within the vector; − indicates the absence of hnRNP A1 in the vector. X-axis: Condition. FIG. 15B shows a graph of relative SNCA protein levels in following delivery of gRNA1, gRNA2, or gRNA, and control gRNA to human neurons relative to control RNA. Y-axis: Relative SNCA protein level; X-axis: Conditions; + indicates presence of two hnRNP A1 within the vector; − indicates the absence of hnRNP A1 in the vector.

FIGS. 16A-16C show that treatment of NSC-derived and iPSC-derived neurons with SNCA TIS guides contributes to exon skipping of exon 2. FIG. 16A shows a gel of PCR amplified SNCA transcription products in NSC-derived neurons. FIG. 16B shows a graph of the percentage of SNCA transcripts in the NSC-derived neurons that display SNCA exon 2 skipping in response to gRNA treatment (ddPCR). Y-axis: % Exon 2 skipping; X-axis: Conditions; + indicates presence of two hnRNP A1 within the vector; − indicates the absence of hnRNP A1 in the vector. 2 replicates per condition were tested. FIG. 16C shows a gel of PCR amplified SNCA transcription products in iPSC-derived neurons.

FIG. 17 shows a schematic of a droplet digital PCR (ddPCR) assay for SNCA novel splice variants. The assay was validated for use with hSNCA mice and human cell lines. The assay measures the amount of transcript with a novel splice junction between hSNCA exons 1 and 3 relative to the canonical exon 2.

FIG. 18 shows a schematic of gRNA modifications to be tested to minimize off-target effects at the −4 position of the targeting portion of the gRNA sequence. The gRNAs were modified to 1. Move the barbell closer to the target mismatch site; 2. Increase the size of the barbell; and introduce U-deletions at the site of the −4 position; and any combination of 1.-4.

FIG. 19 shows a graph of RNA editing by SNCA-TIS-targeting gRNAs in-vitro. Y-axis: % RNA editing; X-axis: Guide Architecture. Parent—P0: SEQ ID NO: 295; Progeny—P15: SEQ ID NO: 333, P16: SEQ ID NO: 390, P18: SEQ ID NO: 334, P20: SEQ ID NO: 335, P22: SEQ ID NO: 391, P24: SEQ ID NO: 336, P26: SEQ ID NO: 392, P28: SEQ ID NO: 393, P28: SEQ ID NO: 394, Structurally Diverse: SEQ ID NO: 395.

### DETAILED DESCRIPTION

### RNA Editing

RNA editing refers to a process by which RNA is enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from: cytidine to give rise to uridine, or C-to-U editing; or from adenosine to inosine, or A-to-I editing). RNA editing can be used to correct mutations (e.g., correction of a missense mutation) in order to restore protein expression and to introduce mutations or edit coding regions of RNA to effect protein knockdown.

Described herein are engineered guide RNAs that facilitate RNA editing by an RNA editing entity (e.g., an adenosine Deaminase Acting on RNA (ADAR)) or biologically active fragments thereof. For example, engineered guide RNAs of the present disclosure can facilitate editing of a transcription initiation site (e.g. the Codon 1 transcription initiation site) of a target SNCA mRNA (for example, an engineered guide RNA of any one SEQ ID NOS: 2-285, 293-299, 333-357, 375-376, 380-381, 390-440, or 777-784). In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain.

The engineered guide RNAs (e.g. an engineered guide RNA that comprises a polynucleotide sequence of any one of SEQ ID NOS: 2-285, 293-299, 333-357, 375-376, 380-381, 390-440, or 777-784) of the present disclosure facilitate RNA editing (for example, of an SNCA Exon 2 Codon 1 transcription initiation site) by endogenous ADAR enzymes. In some embodiments, the engineered guide RNA of the present disclosure can be encoded by a polynucleotide comprising a polynucleotide sequence of any one of SEQ ID NOS: 286-292, 325-332, 358-379, 441-776, or 785-792. In some embodiments, exogenous ADAR can be delivered alongside the engineered guide RNAs disclosed herein to facilitate RNA editing. In some embodiments, the ADAR is human ADAR1. In some embodiments, the ADAR is human ADAR2. In some embodiments, the ADAR is human ADAR3. In some embodiments, the ADAR is human ADAR1, human ADAR2, human ADAR2, or any combination thereof.

TIS. In some embodiments, the engineered guide RNAs of the present disclosure target a target sequence of a target SNCA RNA that comprises a translation initiation site (TIS), wherein the adenosine of the TIS is edited. In some embodiments, an engineered guide RNA of the present disclosure (e.g. an engineered guide RNA that comprises a polynucleotide sequence of any one of SEQ ID NOS: 2-285, 293-299, 333-357, 375-376, 380-381, 390-440, or 777-784 can target the Codon 1 TIS of Exon 2 corresponding to the canonical TIS at nucleotide position 226 of SNCA transcript variant 1 (NCBI Reference Sequence: NM_000345.4). In some embodiments, an engineered guide RNA that targets the SNCA Codon 1 TIS of Exon 2 comprises a polynucleotide sequence of any one of SEQ ID NOS: 293-299, 334, or 340. In some embodiments, an engineered guide disclosed herein is at least partially complementary to a target SNCA RNA. In some cases, the target SNCA RNA comprises the sequence of: GCCAUUCGACGACAGUGUGGUGUAAAGGAAUUCAUUAGCCAUGGAUGUAUU CAUGAAAGGACUUUCAAAGGCCAAGGAGGGAGUUGUGGCUGCUGCUGAG (SEQ ID NO: 300). In some cases, the target SNCA RNA can comprise a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300. An engineered guide RNA of the present disclosure can be used to facilitate modification of the target RNA (e.g., SNCA). In some embodiments, an engineered guide disclosed herein can facilitate ADAR-mediated RNA editing of one or more adenosines in the target RNA sequence of SEQ ID NO: 300. In some embodiments, an engineered guide RNA hybridizes to at least 80 bases of a target RNA sequence with at least 80%, at least 85%, at least 90%, at least 92%, at least 95%, at least 96%, at least 97%, at least 98%, at least 99%, or 100% sequence identity to SEQ ID NO: 300 and facilitates a protein knockdown. In some embodiments, the engineered guide RNAs facilitate ADAR-mediated RNA editing of the TIS (AUG) to GUG. In some instances, this results in protein knockdown. Protein knockdown can also be referred to as reduced expression of wild-type protein. In some instances, an engineered guide disclosed herein can facilitate ADAR-mediated RNA editing of one or more adenosines in the target RNA sequence of SEQ ID NO: 300, resulting in exon skipping. In some cases, the exon skipping that occurs through editing of the SNCA Codon 1 TIS produces SNCA mRNA alternate splice variants. Without wishing to be bound by theory, alternative splicing can result in production of SNCA mRNA alternate splice variants that lack Exon 2, which contains the Codon 1 TIS. The production of these alternative splice variants that lack Exon 2 (and thus the Exon 2 Codon 1 TIS) results in knockdown of wildtype alpha-synuclein protein. Thus, protein knockdown can be accomplished either by directly editing the TIS itself (thereby reducing SNCA mRNA transcription, or by indirectly removing the Exon 2 TIS through exon skipping.

### Engineered Guide RNAs

Disclosed herein are engineered guide RNAs (e.g. an engineered guide RNA that comprise a polynucleotide sequence of any one of SEQ ID NOS: 2-285, 293-299, 333-357, 375-376, 380-381, 390-440, or 777-784) and engineered polynucleotides encoding the same for site-specific, selective editing of a target RNA (for example, an SNCA Codon 1 TIS of Exon 2 corresponding to the canonical TIS at nucleotide position 226 of SNCA transcript variant 1 (NCBI Reference Sequence: NM_000345.4)) via an RNA editing entity or a biologically active fragment thereof. In some embodiments, an engineered guide RNA that targets the SNCA Codon 1 TIS of Exon 2 comprises a polynucleotide sequence of any one of SEQ ID NOS: 293-299, 334, or 340. In some embodiments, the engineered guide RNA of the present disclosure can be encoded by a polynucleotide comprising a polynucleotide sequence of any one of SEQ ID NOS: 286-292, 325-332, 358-379, 441-776, or 785-792. In some embodiments, the engineered guide RNAs of the present disclosure target one or more adenosines in the RNA sequence of SEQ ID NO: 300 or a sequence that is at least 80% identical to SEQ ID NO: 300.

In some embodiments, engineered guide RNAs of the present disclosure that target SNCA comprise a micro-footprint sequence and/or a macro-footprint sequence that each comprise latent structures, such that when the engineered guide RNA is hybridized to the target RNA, the latent structures manifest. A latent structure, when manifested, produces at least one structural feature selected from the group consisting of: a bulge, an internal loop, a mismatch, a hairpin, and any combination thereof. In some embodiments, the engineered guide RNA of the disclosure, upon hybridization of the engineered guide RNA and the sequence of the target RNA form a guide-target RNA scaffold, comprising (i) a region that comprises at least one structural feature; and (ii) a macro-footprint, such as a first internal loop (also referred to as a “left bell” or “LB”) and a second internal loop (also referred to as a “right bell” or “RB”) that flank opposing ends of the region of the guide-target RNA scaffold, where the engineered guide RNA facilitates an increase in the amount of the targeted edit of the adenosine of the target RNA via the adenosine deaminase enzyme RNA editing entity, relative to an otherwise comparable engineered guide RNA lacking the first internal loop and the second internal loop. As described herein, a first internal loop and a second internal loop can be described with respect to their position relative to an A/C mismatch in the target RNA scaffold, where the A in the A/C mismatch is the target adenosine of the SNCA target RNA.

Provided herein are micro-footprint sequences that are latent structures that, when manifested, facilitate editing of the adenosine of a target RNA via an adenosine deaminase enzyme. A macro-footprint can serve to guide an RNA editing entity (e.g., ADAR) and direct its activity towards a micro-footprint. In some embodiments, included within the micro-footprint sequence is a nucleotide that is positioned such that, when the guide RNA is hybridized to the target RNA, the nucleotide opposes the adenosine to be edited by the adenosine deaminase and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA (such as SNCA mRNA).

In some embodiments, guide RNAs of the present disclosure (e.g., a guide RNA that comprises a polynucleotide sequence of any one of SEQ ID NOS: 2-285, 293-299, 333-357, 375-376, 380-381, 390-440, or 777-784) can further comprise a macro-footprint. In some embodiments, the macro-footprint comprises a barbell macro-footprint. A micro-footprint can serve to guide an RNA editing enzyme and direct its activity towards the target adenosine to be edited. A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the SNCA target RNA, results in formation of barbell internal loops on opposing sides of the micro-footprint, which in turn comprises at least one structural feature that facilitates editing of the SNCA target RNA.

Provided herein are engineered guide RNAs (such as latent guide RNA that comprise a micro-footprint sequence and/or a macro-footprint sequence) and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding for engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

An engineered guide RNA as described herein comprises a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule (e.g. an SNCA Codon 1 TIS of Exon 2 corresponding to the canonical TIS at nucleotide position 226 of SNCA transcript variant 1 (NCBI Reference Sequence: NM_000345.4)). A targeting sequence can also be referred to as a “targeting domain” or a “targeting region”.

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

The targeting sequence can have sufficient complementarity to a target RNA to allow for hybridization of the targeting sequence to the target RNA. In some embodiments, the targeting sequence has a minimum antisense complementarity of about 50 nucleotides or more to the target RNA. In some embodiments, the targeting sequence has a minimum antisense complementarity of about 60 nucleotides or more to the target RNA. In some embodiments, the targeting sequence has a minimum antisense complementarity of about 70 nucleotides or more to the target RNA. In some embodiments, the targeting sequence has a minimum antisense complementarity of about 80 nucleotides or more to the target RNA. In some embodiments, the targeting sequence has a minimum antisense complementarity of about 90 nucleotides or more to the target RNA. In some embodiments, the targeting sequence has a minimum antisense complementarity of about 100 nucleotides or more to the target RNA. In some embodiments, antisense complementarity refers to non-contiguous stretches of sequence. In some embodiments, antisense complementarity refers to contiguous stretches of sequence.

In some cases, an engineered guide RNA targeting SNCA can comprise multiple targeting sequences. In some instances, one or more target sequence domains in the engineered guide RNA can bind to one or more regions of a target SNCA RNA. For example, a first targeting sequence can be configured to be at least partially complementary to a first region of a target RNA (e.g., a first exon of a pre-mRNA), while a second targeting sequence can be configured to be at least partially complementary to a second region of a target RNA (e.g. a second exon of a pre-mRNA). In some instances, multiple target sequences can be operatively linked to provide continuous hybridization of multiple regions of a target RNA. In some instances, multiple target sequences can provide non-continuous hybridization of multiple regions of a target RNA. A “non-continuous” overlap or hybridization refers to hybridization of a first region of a target SNCA RNA by a first targeting sequence, along with hybridization of a second region of a target SNCA RNA by a second targeting sequence, where the first region and the second region of the target SNCA RNA are discontinuous (e.g., where there is intervening sequence between the first and the second region of the target RNA). For example, a targeting sequence can be configured to bind to a portion of a first exon and can comprise an internal asymmetric loop (e.g., an oligo tether) that is configured to bind to a portion of a second exon, while the intervening sequence between the portion of exon 1 and the portion of exon 2 is not hybridized by either the targeting sequence or the oligo tether. Use of an engineered guide RNA as described herein configured for non-continuous hybridization can provide a number of benefits. For instance, such a guide can potentially target pre-mRNA during transcription (or shortly thereafter), which can then facilitate chemical modification using a deaminase (e.g., ADAR) co-transcriptionally and thus increase the overall efficiency of the chemical modification. Further, the use of oligo tethers to provide non-continuous hybridization while skipping intervening sequence can result in shorter, more specific guide RNA with fewer off-target editing.

In some instances, an engineered guide RNA configured for non-continuous hybridization to a target SNCA RNA (e.g., an engineered guide RNA comprising a targeting sequence with an oligo tether) can be configured to bind distinct regions or a target SNCA RNA separated by intervening sequence. In some instances, the intervening sequence can be at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 nucleotides. In some instances, the targeting sequence and oligo tether can target distinct non-continuous regions of the same intron or exon. In some instances, the targeting sequence and oligo tether can target distinct non-continuous regions of adjacent exons or introns. In some instances, the targeting sequence and oligo tether can target distinct non-continuous regions of distal exons or introns.

In some embodiments, a guide RNA or a polynucleotide encoding a guide RNA disclosed herein can comprise a targeting sequence disclosed in Table 2, such as any one of SEQ ID NOS: 293-299, 333-357 (represented as DNA sequences in SEQ ID NOS: 286-292, 325-332, 358-374). In some embodiments, a composition can comprise an engineered guide RNA comprising any one of SEQ ID NOS: 293-299, 333-357. In some embodiments, a composition can comprise an engineered guide RNA comprising any one of SEQ ID NOS: 293-299, 334, or 340. In some embodiments, a composition can comprise an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to any one of SEQ ID NOS: 293-299, 333-357. In some embodiments, a composition can comprise an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99%, sequence identity to any one of SEQ ID NOS: 293-299, 334, or 340. In some embodiments, a composition can comprise a polynucleotide encoding an engineered guide RNA comprising any one of SEQ ID NOS: 293-299, 333-357. In some embodiments, a composition can comprise a polynucleotide encoding an engineered guide RNA comprising any one of SEQ ID NOS: 293-299, 334, or 340. In some embodiments, a composition can comprise a polynucleotide encoding an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99% sequence identity to any one of any one of SEQ ID NOS: 293-299, 333-357. In some embodiments, a composition can comprise a polynucleotide encoding an engineered guide RNA with at least about: 80%, 81%, 82%, 83%, 84%, 85%, 86%, 87%, 88%, 89%, 90%, 91%, 92%, 93%, 94%, 95%, 96%, 97%, 98%, or 99% sequence identity to any one of any one of SEQ ID NOS: 293-299, 334, or 340.

In some embodiments, hybridization of a targeting domain of an engineered guide RNA to a target SNCA RNA results in protein knockdown. For example, hybridization of a targeting domain of an engineered guide RNA to a sequence of a target SNCA RNA containing the Exon 2 Codon 1 TIS can result in silencing of the Codon 1 TIS. This silencing can occur due to, for example, where the hybridization of the targeting domain to the target SNCA RNA results in ADAR-mediated editing of the adenosine of the Codon 1 TIS, thus converting the AUG start codon to a GUG codon and silencing the Codon 1 TIS. Silencing can also occur for to, for example, where the hybridization of the targeting domain to the target SNCA RNA results in exon skipping of Exon 2, thus silencing the Exon 2 TIS by removing it from the mature SNCA mRNA. In some cases, silencing of the Exon 2 Codon 1 TIS can occur due to both ADAR-mediated editing of the Exon 2 TIS and exon skipping of Exon 2.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can facilitate editing of a base of a nucleotide of in a target sequence of a target RNA (e.g., SEQ ID NO: 300) that results in modulating the expression of a polypeptide encoded by the target RNA. Said modulation can be increased expression of the polypeptide or decreased expression of the polypeptide. In some cases, an engineered guide can be configured to facilitate an editing of a base of a nucleotide or polynucleotide of a region of an RNA by an RNA editing entity (e.g., ADAR). In order to facilitate editing, an engineered guide RNA of the disclosure can recruit an RNA editing entity (e.g., ADAR). Various RNA editing entity recruiting domains can be utilized. In some examples, a recruiting domain comprises: Glutamate ionotropic receptor AMPA type subunit 2 (GluR2), an Alu sequence, or, in the case of recruiting APOBEC, an APOBEC recruiting domain.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence hybridizes to a target sequence of a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In some embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some examples, a recruiting domain can comprise at least about 90%, 95%, 96%, 97%, 98%, or 99% sequence homology and/or length to SEQ ID NO: 1.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of engineered guide RNAs. In some cases, a recruiting domain can be on an N-terminus, middle, or C-terminus of an engineered guide RNA. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribonucleotides and deoxyribonucleotides can in some cases not be excluded.

C. Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA (for example, an SNCA Codon 1 TIS). The resulting dsRNA substrate is also referred to herein as a “guide-target RNA scaffold.”

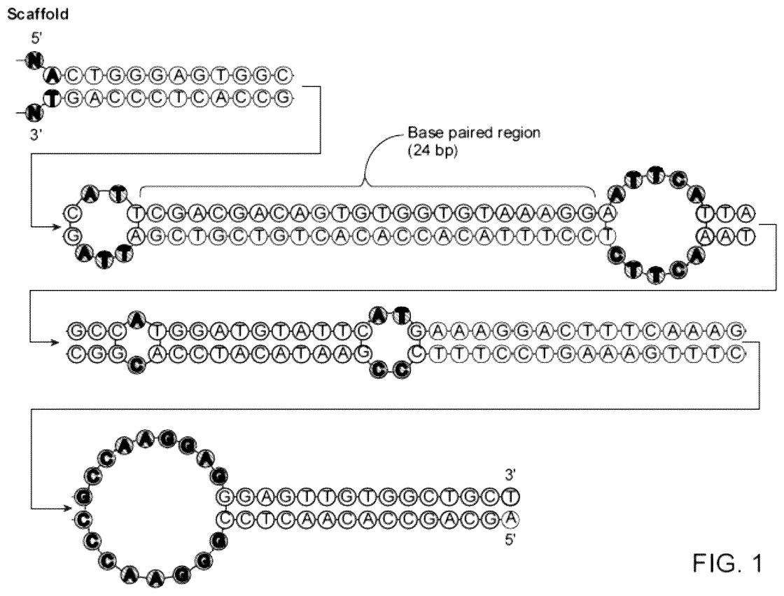

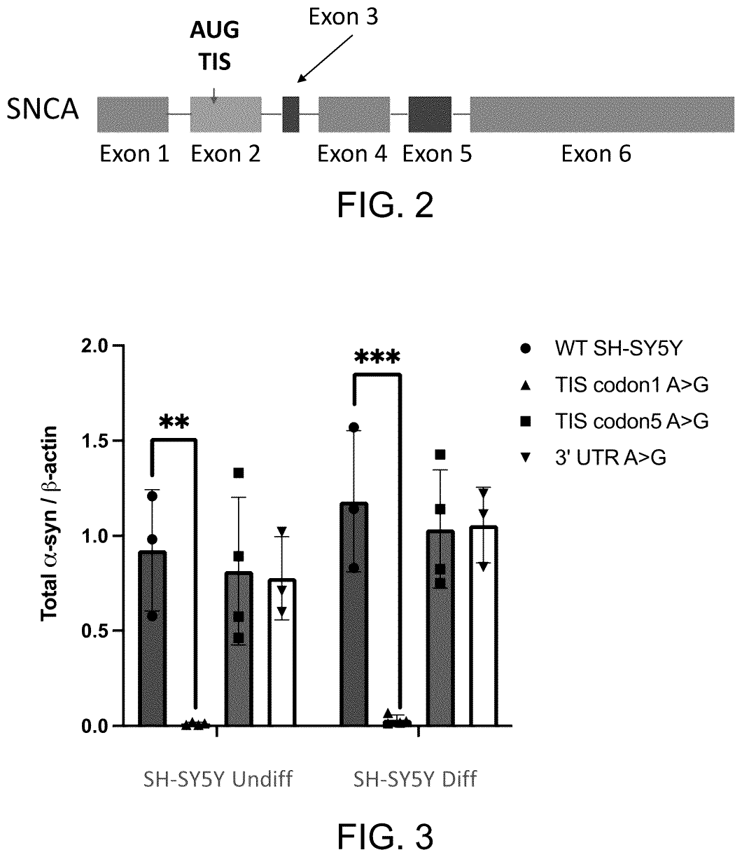

FIG. 1 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a 2/3 asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the 2/3 asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a 2/3 asymmetric bulge at position −36, then the 2/3 asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position +12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence can be absent, an engineered guide RNA can be still capable of associating with a subject RNA editing entity (e.g., ADAR) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

A double stranded RNA (dsRNA) substrate is formed upon hybridization of an engineered guide RNA of the present disclosure to a target RNA. The resulting dsRNA substrate is also referred to herein as a “guide-target RNA scaffold.” Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A double stranded RNA (dsRNA) substrate (i.e., a guide-target RNA scaffold) is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA.

In some embodiments, a mismatch positioned 5′ of the edit site can facilitate base-flipping of the target A to be edited. A mismatch can also help confer sequence specificity.

Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in-vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A double stranded RNA (dsRNA) substrate (i.e., a guide-target RNA scaffold) is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A double stranded RNA (dsRNA) substrate (i.e., a guide-target RNA scaffold) is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some embodiments, an asymmetric bulge can be a I/O asymmetric bulge. In some embodiments, a I/O asymmetric bulge can be a U deletion. A “U deletion” refers to a I/O asymmetric bulge in which a U nucleotide of an engineered guide RNA that would be situated opposite a non-target A of a target RNA in the guide-target RNA scaffold is deleted from the engineered guide RNA. In some instances, a I/O asymmetric bulge comprising a U deletion can reduce editing of the non-target A, relative to a comparable guide RNA lacking the U deletion.

A double stranded RNA (dsRNA) substrate (i.e., a guide-target RNA scaffold) is formed upon hybridization of an engineered guide RNA of the present disclosure to a target RNA. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides there between. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 15 nucleotides on the engineered polynucleotide side of the guide-target RNA scaffold target and 15 nucleotides on the target RNA side of the guide-target RNA scaffold. A symmetrical internal loop of the present disclosure can be formed by 20 nucleotides on the engineered polynucleotide side of the guide-target RNA scaffold target and 20 nucleotides on the target RNA side of the guide-target RNA scaffold. A symmetrical internal loop of the present disclosure can be formed by 30 nucleotides on the engineered polynucleotide side of the guide-target RNA scaffold target and 30 nucleotides on the target RNA side of the guide-target RNA scaffold. A symmetrical internal loop of the present disclosure can be formed by 40 nucleotides on the engineered polynucleotide side of the guide-target RNA scaffold target and 40 nucleotides on the target RNA side of the guide-target RNA scaffold. A symmetrical internal loop of the present disclosure can be formed by 50 nucleotides on the engineered polynucleotide side of the guide-target RNA scaffold target and 50 nucleotides on the target RNA side of the guide-target RNA scaffold. A symmetrical internal loop of the present disclosure can be formed by 60 nucleotides on the engineered polynucleotide side of the guide-target RNA scaffold target and 60 nucleotides on the target RNA side of the guide-target RNA scaffold. A symmetrical internal loop of the present disclosure can be formed by 70 nucleotides on the engineered polynucleotide side of the guide-target RNA scaffold target and 70 nucleotides on the target RNA side of the guide-target RNA scaffold. A symmetrical internal loop of the present disclosure can be formed by 80 nucleotides on the engineered polynucleotide side of the guide-target RNA scaffold target and 80 nucleotides on the target RNA side of the guide-target RNA scaffold. A symmetrical internal loop of the present disclosure can be formed by 90 nucleotides on the engineered polynucleotide side of the guide-target RNA scaffold target and 90 nucleotides on the target RNA side of the guide-target RNA scaffold. A symmetrical internal loop of the present disclosure can be formed by 100 nucleotides on the engineered polynucleotide side of the guide-target RNA scaffold target and 100 nucleotides on the target RNA side of the guide-target RNA scaffold. A symmetrical internal loop of the present disclosure can be formed by 110 nucleotides on the engineered polynucleotide side of the guide-target RNA scaffold target and 110 nucleotides on the target RNA side of the guide-target RNA scaffold. A symmetrical internal loop of the present disclosure can be formed by 120 nucleotides on the engineered polynucleotide side of the guide-target RNA scaffold target and 120 nucleotides on the target RNA side of the guide-target RNA scaffold. A symmetrical internal loop of the present disclosure can be formed by 130 nucleotides on the engineered polynucleotide side of the guide-target RNA scaffold target and 130 nucleotides on the target RNA side of the guide-target RNA scaffold. A symmetrical internal loop of the present disclosure can be formed by 140 nucleotides on the engineered polynucleotide side of the guide-target RNA scaffold target and 140 nucleotides on the target RNA side of the guide-target RNA scaffold. A symmetrical internal loop of the present disclosure can be formed by 150 nucleotides on the engineered polynucleotide side of the guide-target RNA scaffold target and 150 nucleotides on the target RNA side of the guide-target RNA scaffold. A symmetrical internal loop of the present disclosure can be formed by 200 nucleotides on the engineered polynucleotide side of the guide-target RNA scaffold target and 200 nucleotides on the target RNA side of the guide-target RNA scaffold. A symmetrical internal loop of the present disclosure can be formed by 250 nucleotides on the engineered polynucleotide side of the guide-target RNA scaffold target and 250 nucleotides on the target RNA side of the guide-target RNA scaffold. A symmetrical internal loop of the present disclosure can be formed by 300 nucleotides on the engineered polynucleotide side of the guide-target RNA scaffold target and 300 nucleotides on the target RNA side of the guide-target RNA scaffold. A symmetrical internal loop of the present disclosure can be formed by 350 nucleotides on the engineered polynucleotide side of the guide-target RNA scaffold target and 350 nucleotides on the target RNA side of the guide-target RNA scaffold. A symmetrical internal loop of the present disclosure can be formed by 400 nucleotides on the engineered polynucleotide side of the guide-target RNA scaffold target and 400 nucleotides on the target RNA side of the guide-target RNA scaffold. A symmetrical internal loop of the present disclosure can be formed by 450 nucleotides on the engineered polynucleotide side of the guide-target RNA scaffold target and 450 nucleotides on the target RNA side of the guide-target RNA scaffold. A symmetrical internal loop of the present disclosure can be formed by 500 nucleotides on the engineered polynucleotide side of the guide-target RNA scaffold target and 500 nucleotides on the target RNA side of the guide-target RNA scaffold. A symmetrical internal loop of the present disclosure can be formed by 600 nucleotides on the engineered polynucleotide side of the guide-target RNA scaffold target and 600 nucleotides on the target RNA side of the guide-target RNA scaffold. A symmetrical internal loop of the present disclosure can be formed by 700 nucleotides on the engineered polynucleotide side of the guide-target RNA scaffold target and 700 nucleotides on the target RNA side of the guide-target RNA scaffold. A symmetrical internal loop of the present disclosure can be formed by 800 nucleotides on the engineered polynucleotide side of the guide-target RNA scaffold target and 800 nucleotides on the target RNA side of the guide-target RNA scaffold. A symmetrical internal loop of the present disclosure can be formed by 900 nucleotides on the engineered polynucleotide side of the guide-target RNA scaffold target and 900 nucleotides on the target RNA side of the guide-target RNA scaffold. A symmetrical internal loop of the present disclosure can be formed by 1000 nucleotides on the engineered polynucleotide side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

A double stranded RNA (dsRNA) substrate (i.e., a guide-target RNA scaffold) is formed upon hybridization of an engineered guide RNA of the present disclosure to a target RNA. An internal loop can be a symmetrical internal loop or an asymmetrical internal loop. An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered polynucleotide side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered polynucleotide side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

The present disclosure provides engineered guide RNAs (for example, an engineered guide RNA comprising a polynucleotide sequence of any one of SEQ ID NOS: 2-285, 293-299, 333-357, 375-376, 380-381, 390-440, or 777-784) that target a sequence of an SNCA target RNA (for example, the Codon 1 TIS of Exon 2 corresponding to the canonical TIS at nucleotide position 226 of SNCA transcript variant 1 (NCBI Reference Sequence: NM_000345.4)). In some embodiments, the target RNA comprises the sequence of SEQ ID NO: 300. In some cases, the target RNA can comprise a sequence with at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 300.

In some embodiments, an engineered guide RNA of the present disclosure that targets the SNCA Codon 1 TIS in Exon 2 comprises one or more structural features, where the one or more structural features comprises at least a first 6/6 symmetric internal loop and at least a second 6/6 symmetric loop. In some embodiments, the first 6/6 symmetric internal loop is at a position selected from the group consisting of: 33, 32, 30, 28, and 26; relative to the target adenosine at position 0.

In some embodiments, the first 6/6 symmetric internal loop is at position 33, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, 99%, or 100% sequence identity to SEQ ID NO: 337.

In some embodiments, the first 6/6 symmetric internal loop is at position 33, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, 99%, or 100% sequence identity to SEQ ID NO: 338 or SEQ ID NO: 339.

In some embodiments, the first 6/6 symmetric internal loop is at position 33, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, an A/C mismatch at position 0, a G/U wobble base at position 2 relative to position 0, a U/G wobble base at position 5 relative to position 0, a 2/2 symmetric bulge at position 33 relative to position 0, a G/G mismatch at position 36 relative to position 0, a G/G mismatch at position 38 relative to position 0, and any combination thereof. In some embodiments, the engineered guide RNA comprises at least about: 80%, 85%, 90%, 92%, 95%, 97%, 99%, or 100% sequence identity to SEQ ID NO: 340.

In some embodiments, the first 6/6 symmetric internal loop is at position 33, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, and any combination thereof. In some embodiments, the engineered guide RNA comprises at least about: 80%, 85%, 90%, 92%, 95%, 97%, 99%, or 100% sequence identity to SEQ ID NO: 298 or SEQ ID NO: 299.

In some embodiments, the first 6/6 symmetric internal loop is at position 33, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof. In some embodiments, the engineered guide RNA comprises at least about: 80%, 85%, 90%, 92%, 95%, 97%, 99%, or 100% sequence identity to SEQ ID NO: 299.

In some embodiments, the first 6/6 symmetric internal loop is at position 32, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, 99%, or 100% sequence identity to SEQ ID NO: 333.

In some embodiments, the first 6/6 symmetric internal loop is at position 32, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, 99%, or 100% sequence identity to SEQ ID NO: 334.

In some embodiments, the first 6/6 symmetric internal loop is at position 32, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, 99%, or 100% sequence identity to SEQ ID NO: 335.

In some embodiments, the first 6/6 symmetric internal loop is at position 32, relative to the target adenosine at position 0. In some embodiments, the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, a G/U wobble base at position −3 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof. In some embodiments, the engineered guide RNA comprises at least about: 80%, 85%, 90%, 92%, 95%, 97%, 99%, or 100% sequence identity to SEQ ID NO: 336.

In some embodiments, the first 6/6 symmetric internal loop is at position 32, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, and any combination thereof. In some embodiments, the engineered guide RNA comprises at least about: 80%, 85%, 90%, 92%, 95%, 97%, 99%, or 100% sequence identity to SEQ ID NO: 295.

In some embodiments, the first 6/6 symmetric internal loop is at position 30, relative to the target adenosine at position 0. In some embodiments, the one or more structural features further comprises at least one structural feature selected from the group consisting of: the second 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof. In some embodiments, the engineered guide RNA comprises at least about: 80%, 85%, 90%, 92%, 95%, 97%, 99%, or 100% sequence identity to SEQ ID NO: 297.

In some embodiments, the first 6/6 symmetric internal loop is at position 28, relative to the target adenosine at position 0. In some embodiments, the one or more structural features further comprises at least one structural feature selected from the group consisting of: the second 6/6 symmetric internal loop at position −8 relative to position 0, an A/C mismatch at position 0, a G/U wobble base pair at position 2 relative to position 0, and any combination thereof. In some embodiments, the engineered guide RNA comprises at least about: 80%, 85%, 90%, 92%, 95%, 97%, 99%, or 100% sequence identity to SEQ ID NO: 293.

In some embodiments, the first 6/6 symmetric internal loop is at position 28, relative to the target adenosine at position 0. In some embodiments, the one or more structural features further comprises at least one structural feature selected from the group consisting of: the second 6/6 symmetric internal loop at position −10 relative to position 0, a 0/1 asymmetric bulge at position −6 relative to position 0, an A/C mismatch at position 0, an A/A mismatch at position 4 relative to position 0, and any combination thereof. In some embodiments, the engineered guide RNA comprises at least about: 80%, 85%, 90%, 92%, 95%, 97%, 99%, or 100% sequence identity to SEQ ID NO: 294.

In some embodiments, the first 6/6 symmetric internal loop is at position 26, relative to the target adenosine at position 0. In some embodiments, the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof. In some embodiments, the engineered guide RNA comprises at least about: 80%, 85%, 90%, 92%, 95%, 97%, 99%, or 100% sequence identity to SEQ ID NO: 296.

D. Guides with Macro-Footprints

Guide RNAs of the present disclosure can further comprise a macro-footprint. In some embodiments, the macro-footprint comprises a barbell macro-footprint. A micro-footprint can serve to guide an RNA editing enzyme and direct its activity towards the target adenosine to be edited. A “barbell” as described herein refers to a pair of internal loop latent structures that manifest upon hybridization of the guide RNA to the target RNA. In some embodiments, each internal loop is positioned towards the 5′ end or the 3′ end of the guide-target RNA scaffold formed upon hybridization of the guide RNA and the target RNA. In some embodiments, each internal loop flanks opposing sides of the micro-footprint sequence. Insertion of a barbell macro-footprint sequence flanking opposing sides of the micro-footprint sequence, upon hybridization of the guide RNA to the target RNA, results in formation of barbell internal loops on opposing sides of the micro-footprint. In some cases, barbell internal loops can comprise at least one structural feature that facilitates editing of a specific target RNA.

In some embodiments, the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the increase in the one or more aspects of editing provided by the barbell macro-footprint structures can be independent of the particular target RNA. For example, macro-footprints (e.g., barbell macro-footprints) and micro-footprints can provide an increased amount of on target adenosine editing relative to an otherwise comparable guide RNA lacking the barbells. In other embodiments, the presence of the barbell macro-footprint in addition to the micro-footprint described here can result in a lower amount of local off-target adenosine editing, relative to an otherwise comparable guide RNA, upon hybridization of the guide RNA and target RNA to form a guide-target RNA scaffold lacking the barbells.

As described herein, a “micro-footprint” sequence refers to a sequence with latent structures that, when manifested, facilitate editing of the adenosine of a target RNA via an adenosine deaminase enzyme. A macro-footprint can serve to guide an or focus RNA editing entity (e.g., ADAR) and direct its activity towards a micro-footprint. In some embodiments, included within the micro-footprint sequence is a nucleotide that is positioned such that, when the guide RNA is hybridized to the target RNA, said nucleotide is opposite the adenosine to be edited by the ADAR enzyme and does not base pair with the adenosine to be edited. This nucleotide is referred to herein as the “mismatched position” or “mismatch” and can be a cytosine. Micro-footprint sequences as described herein have upon hybridization of the engineered guide RNA and target RNA, at least one structural feature selected from the group consisting of: a bulge, an internal loop, a mismatch, a hairpin, and any combination thereof. Engineered guide RNAs with superior micro-footprint sequences can be selected based on their ability to facilitate editing of a specific target RNA. Engineered guide RNAs selected for their ability to facilitate editing of a specific target are capable of adopting various micro-footprint latent structures, which can vary on a target-by-target basis.

In some embodiments, the presence of barbells flanking the micro-footprint can improve one or more aspects of editing. For example, the presence of a barbell macro-footprint in addition to a micro-footprint can result in a higher amount of on target adenosine editing, relative to an otherwise comparable guide RNA lacking the barbells. Additionally, and or alternatively, the presence of a barbell macro-footprint in addition to a micro-footprint can result in a lower amount of local off-target adenosine editing, relative to an otherwise comparable guide RNA lacking the barbells. Further, while the effect of various micro-footprint structural features can vary on a target-by-target basis based on selection in a high throughput screen, the increase in the one or more aspects of editing provided by the barbell macro-footprint structures can be independent of the particular target RNA. For example, macro-footprints (e.g., barbell macro-footprints) and micro-footprints can provide an increased amount of on target adenosine editing relative to an otherwise comparable guide RNA lacking the barbells. In other embodiments, the presence of the barbell macro-footprint in addition to the micro-footprint described here can result in a lower amount of local off-target adenosine editing, relative to an otherwise comparable guide RNA, upon hybridization of the guide RNA and target RNA to form a guide-target RNA scaffold lacking the barbells.

A dumbbell design in an engineered guide RNA comprises two symmetrical internal loops, wherein the target A to be edited is positioned between the two symmetrical loops for selective editing of the target A. The two symmetrical internal loops are each formed by 6 nucleotides on the guide RNA side of the guide-target RNA scaffold and 6 nucleotides on the target RNA side of the guide-target RNA scaffold. Thus, a dumbbell can be a structural feature formed from latent structure provided by an engineered latent guide RNA.

As disclosed herein, a “macro-footprint” sequence can be positioned such that it flanks a micro-footprint sequence. Further, while a macro-footprint sequence can flank a micro-footprint sequence, additional latent structures can be incorporated that flank either end of the macro-footprint as well. In some embodiments, such additional latent structures are included as part of the macro-footprint. In some embodiments, such additional latent structures are separate, distinct, or both separate and distinct from the macro-footprint. In some embodiments, a macro-footprint sequence can comprise a barbell macro-footprint sequence comprising latent structures that, when manifested, produce a first internal loop and a second internal loop.

In some embodiments, the first internal loop of the barbell or the second internal loop of the barbell is positioned at least about 5 bases (e.g., 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bases) away from the A/C mismatch with respect to the base of the first internal loop or the second internal loop that is the most proximal to the A/C mismatch. In some embodiments, the first internal loop of the barbell or the second internal loop of the barbell is positioned at most about 50 bases away from the A/C mismatch (e.g., 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5) with respect to the base of the first internal loop or the second internal loop that is the most proximal to the A/C mismatch.

In some embodiments, a first internal loop or a second internal loop independently comprises a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the engineered guide RNA and a number of bases of at least about 5 bases or greater (e.g., 6, 7, 8, 9, 10, 11, 12, 13, 14, 15, 16, 17, 18, 19, 20, 30, 40, 50, 60, 70, 80, 90, 100, 110, 120, 130, 140, 150); about 150 bases or fewer (e.g., 145, 135, 125, 115, 95, 85, 75, 65, 55, 45, 35, 25, 19, 18, 17, 16, 15, 14, 13, 12, 11, 10, 9, 8, 7, 6, 5); or at least about 5 bases to at least about 150 bases (e.g., 5-150, 6-145, 7-140, 8-135, 9-130, 10-125, 11-120, 12-115, 13-110, 14-105, 15-100, 16-95, 17-90, 18-85, 19-80, 20-75, 21-70, 22-65, 23-60, 24-55, 25-50) of the target RNA.

In some embodiments, provided herein are engineered guide RNAs comprising a barbell macro-footprint. In some embodiments, provided herein are engineered guide RNAs comprising a micro-footprint. In some embodiments, provided herein are engineered guide RNAs comprising a macro-footprint and a micro-footprint. In some cases, an engineered guide RNA disclosed herein can comprise a micro-footprint in the absence of a macro-footprint. In some cases, an engineered guide RNA disclosed herein can comprise a macro-footprint in the absence of a micro-footprint.

In some embodiments, a macro-footprint sequence can comprise a barbell macro-footprint sequence comprising latent structures that, when manifested, produce a first internal loop and a second internal loop.

In some examples, a first internal loop is positioned near the 5′ end of the guide-target RNA scaffold and a second internal loop is positioned near the 3′ end of the guide-target RNA scaffold. The length of the dsRNA comprises a 5′ end and a 3′ end, where up to half of the length of the guide-target RNA scaffold at the 5′ end can be considered to be “near the 5′ end” while up to half of the length of the guide-target RNA scaffold at the 3′ end can be considered “near the 3′ end.” Non-limiting examples of the 5′ end can include about 50% or less of the total length of the dsRNA at the 5′ end, about 45%, about 40%, about 35%, about 30%, about 25%, about 20%, about 15%, about 10%, or about 5%. Non-limiting examples of the 3′ end can include about 50% or less of the total length of the dsRNA at the 3′ end about 45%, about 40%, about 35%, about 30%, about 25%, about 20%, about 15%, about 10%, or about 5%.

In some embodiments, the engineered guide RNAs of the disclosure comprising a barbell macro-footprint sequence (that manifests as a first internal loop and a second internal loop) can improve RNA editing efficiency, increase the amount or percentage of RNA editing generally, as well as for on-target nucleotide editing, such as on-target adenosine. In some embodiments, the engineered guide RNAs of the disclosure comprising a first internal loop and a second internal loop can also facilitate a decrease in the amount of or reduce off-target nucleotide editing, such as off-target adenosine or unintended adenosine editing. The decrease or reduction in some examples can be of the number of off-target edits or the percentage of off-target edits.

Each of the first and second internal loops of the barbell macro-footprint can independently be symmetrical or asymmetrical, where symmetry is determined by the number of bases or nucleotides of the engineered guide RNA and the number of bases or nucleotides of the target RNA, that together form each of the first and second internal loops.

### E. Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both sequences.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl. In some embodiments, a circular engineered guide RNA can comprise a guide RNA comprising a polynucleotide sequence of any one of SEQ ID NOS: 2-285, 293-299, 333-357, 375-376, 380-381, 390-440, or 777-784.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. For example, PCT/US2021/034301 provides a description of circular guide RNAs and their structures, sequences of circular guide RNAs, and methods of engineering circularized polynucleotide domains, and each of these descriptions in PCT/US2021/034301 is herein incorporated by reference.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g., increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

A circular or looped RNA can be formed by employing a self-cleaving entity, such as a ribozyme, tRNA, aptamer, catalytically active fragment of any of these, or any combination thereof. For example, a ribozyme, a tRNA, an aptamer, a catalytically active fragment of any of these, or any combination thereof can be added to a 3′ end, a 5′ end, or both of a precursor engineered RNA. In another example, a ribozyme, a tRNA, an aptamer, a catalytically active fragment of any of these, or any combination thereof can be added to a 3′ terminal end, a 5′ terminal end, or both of a precursor engineered RNA. A self-cleaving ribozyme can comprise, for example, an RNase P RNA a Hammerhead ribozyme (e.g., a Schistosoma mansoni ribozyme), a glmS ribozyme, an HDV-like ribozyme, an R2 element, a peptidyl transferase 23S rRNA, a GIRT branching ribozyme, a leadzyme, a group II intron, a hairpin ribozyme, a VS ribozyme, a CPEB3 ribozyme, a CoTC ribozyme, or a group I intron. In some cases, the self-cleaving ribozyme can be a trans-acting ribozyme that joins one RNA end on which it is present to a separate RNA end. In some embodiments, an aptamer can be added to each end of the engineered guide RNA. A ligase can be contacted with the aptamers at each end of the engineered guide RNA to form a covalent linkage between the aptamers thereby forming a circular engineered guide RNA. In some cases, a self-cleaving element or an aptamer can be configured to facilitate self-circularization of an engineered polynucleotide or a pro-polynucleotide (e.g., from a precursor engineered polypeptide) after transcription in a cell. In some instances, circularization of a guide RNA can be shown by PCR. For example, primers can by developed that bind to the end of a guide RNA and are directed outward such that a product is only formed when guides are circularized.

In some cases, circularization can occur by back-slicing and ligation of an exon. For example, an RNA can be engineered from 5′ to 3′ to comprise a forward complementary sequence intron, an exon (which can comprise the guide sequence), followed by a reverse complementary sequence intron. Once transcribed, the complementary sequence introns can hybridize and form dsRNA. The internal exon containing the guide sequence can be removed by splicing and ligated by an endogenous ligase to form a circular guide. In one example, an engineered guide RNA can initiate circularization in a cell by autocatalytic reactions of encoded ribozymes. After cleavage by one or more ribozymes, the linear polynucleotide will undergo intracellular RNA ligation of the 5′ and the 3′ end of ligation sequences by an endogenous ligase to circularize the guide RNA.

A suitable self-cleaving molecule can include a ribozyme. For example, a ribozyme domain can create an autocatalytic RNA. A ribozyme can comprise an RNase P, an rRNA (such as a Peptidyl transferase 23S rRNA), Leadzyme, Group I intron ribozyme, Group II intron ribozyme, a GIR1 branching ribozyme, a glmS ribozyme, a hairpin ribozyme, a Hammerhead ribozyme, an HDV ribozyme, a Twister ribozyme, a Twister sister ribozyme, a VS ribozyme, a Pistol ribozyme, a Hatchet ribozyme, a viroid, or any combination thereof. A ribozyme can include a P3 twister U2A ribozyme. A ribozyme can comprise 5′ GCCATCAGTCGCCGGTCCCAAGCCCGGATAAAATGGGAGGGGGCGGGAAACCGC CT 3′ (SEQ ID NO: 313). A ribozyme can comprise 5′ GCCAUCAGUCGCCGGUCCCAAGCCCGGAUAAAAUGGGAGGGGGCGGGAAACCG CCU 3′ (SEQ ID NO: 314). A ribozyme can comprise at least about: 70%, 75%, 80%, 85%, 90%, 95%, or 100% sequence homology to 5′ GCCATCAGTCGCCGGTCCCAAGCCCGGATAAAATGGGAGGGGGCGGGAAACCGC CT 3′ (SEQ ID NO: 313). A ribozyme can comprise at least about: 70%, 75%, 80%, 85%, 90%, 95%, or 100% sequence homology to 5′ GCCAUCAGUCGCCGGUCCCAAGCCCGGAUAAAAUGGGAGGGGGCGGGAAACCG CCU 3′ (SEQ ID NO: 314). A ribozyme can include a P1 Twister Ribozyme. A ribozyme can include 5′ AACACTGCCAATGCCGGTCCCAAGCCCGGATAAAAGTGGAGGGTACAGTCCACG C 3′ (SEQ ID NO: 317). A ribozyme can include 5′ AACACUGCCAAUGCCGGUCCCAAGCCCGGAUAAAAGUGGAGGGUACAGUCCAC GC 3′ (SEQ ID NO: 318). A ribozyme can comprise at least about: 70%, 75%, 80%, 85%, 90%, 95%, or 100% sequence homology to 5′ AACACTGCCAATGCCGGTCCCAAGCCCGGATAAAAGTGGAGGGTACAGTCCACG C 3′ (SEQ ID NO: 317). A ribozyme can comprise at least about: 70%, 75%, 80%, 85%, 90%, 95%, or 100% sequence homology to 5′ AACACUGCCAAUGCCGGUCCCAAGCCCGGAUAAAAGUGGAGGGUACAGUCCAC GC 3′ (SEQ ID NO: 318).

A ligation domain can facilitate a linkage, covalent or non-covalent, of a first nucleotide to a second nucleotide. In some embodiments, a ligation domain can recruit a ligating entity to facilitate a ligation reaction. In some cases, a ligation domain can recruit a recombining entity to facilitate a homologous recombination. In some instances, a first ligation domain can facilitate a linkage, covalent or non-covalent, to a second ligation domain. In some embodiments, a first ligation domain can facilitate the complementary pairing of a second ligation domain. In some cases, a ligation domain can comprise 5′ AACCATGCCGACTGATGGCAG 3′ (SEQ ID NO: 320). In some embodiments, a ligation domain can comprise 5′ GATGTCAGGTGCGGCTGACTACCGTC 3′ (SEQ ID NO: 321). In some cases, a ligation domain can comprise 5′ AACCAUGCCGACUGAUGGCAG 3′ (SEQ ID NO: 322). In some cases, a ligation domain can comprise 5′ GAUGUCAGGUGCGGCUGACUACCGUC 3′ (SEQ ID NO: 323). In some cases, a ligation domain can comprise at least about: 70%, 75%, 80%, 85%, 90%, 95%, or 100% sequence homology to 5′AACCATGCCGACTGATGGCAG 3′ (SEQ ID NO: 320). In some cases, a ligation domain can comprise at least about: 70%, 75%, 80%, 85%, 90%, 95%, or 100% sequence homology to 5′ GATGTCAGGTGCGGCTGACTACCGTC 3′ (SEQ ID NO: 321). In some cases, a ligation domain can comprise at least about: 70%, 75%, 80%, 85%, 90%, 95%, or 100% sequence homology to 5′ AACCAUGCCGACUGAUGGCAG 3′ (SEQ ID NO: 322). In some cases, a ligation domain can comprise at least about: 70%, 75%, 80%, 85%, 90%, 95%, or 100% sequence homology to 5′ GAUGUCAGGUGCGGCUGACUACCGUC 3′ (SEQ ID NO: 323).

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered guide RNA that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features. Promoters and snRNA components are described in PCT/US2021/028618 and PCT/US2022/078801, and each of these descriptions in PCT/US2021/028618 and PCT/US2022/078801 are herein incorporated by reference.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 301 or RNA: UAGGCUUUCUGGCUUUUUACCGGAAAGCCCCU (SEQ ID NO: 302). In some cases, a mouse U7 hairpin sequence comprises CAGGTTTTCTGACTTCGGTCGGAAAACCCCT (SEQ ID NO: 303 or RNA: CAGGUUUUCUGACUUCGGUCGGAAAACCCCU SEQ ID NO: 304). In some embodiments, the SmOPT sequence has a sequence of AATTTTTGGAG (SEQ ID NO: 305 or RNA: AAUUUUUGGAG SEQ ID NO: 306). In some embodiments, a guide RNA comprising a polynucleotide sequence of any one of SEQ ID NOS: 2-285, 293-299, 333-357, 375-376, 380-381, 390-440, or 777-784 that target SNCA Codon 1 TIS of Exon 2 can comprise a guide RNA comprising a U7 hairpin sequence (e.g., a human or a mouse U7 hairpin sequence), an SmOPT sequence, or a combination thereof. In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

Also disclosed herein are promoters for driving the expression of a guide RNA disclosed herein. In some cases, the promoters for driving expression can be 5′ to the guide RNA sequence disclosed herein. In some cases, a promoter can comprise a U1 promoter, a U7 promoter, a U6 promoter or any combination thereof. In some cases, a promoter can comprise a CMV promoter. In some cases, a U7 promoter, or a U6 promoter can be a mouse U7 promoter, or a mouse U6 promoter. In some cases, a U1 promoter, a U7 promoter, or a U6 promoter can be a human U1 promoter, a human U7 promoter, or a human U6 promoter. In some cases, a human U6 promoter can comprise a sequence with at least about: 70%, 75%, 80%, 85%, 90%, 95%, or 99% sequence identity to: GAGGGCCTATTTCCCATGATTCCTTCATATTTGCATATACGATACAAGGCTGTTA GAGAGATAATTAGAATTAATTTGACTGTAAACACAAAGATATTAGTACAAAATA CGTGACGTAGAAAGTAATAATTTCTTGGGTAGTTTGCAGTTTTAAAATTATGTTTT AAAATGGACTATCATATGCTTACCGTAACTTGAAAGTATTTCGATTTCTTGGCTTT ATATATCTTGTGGAAAGGACGAAACACC (SEQ ID NO: 307). In some cases, a mouse U6 promoter can comprise a sequence with at least about: 70%, 75%, 80%, 85%, 90%, 95%, or 99% sequence identity to: GTACTGAGTCGCCCAGTCTCAGATAGATCCGACGCCGCCATCTCTAGGCCCGCGC CGGCCCCCTCGCACAGACTTGTGGGAGAAGCTCGGCTACTCCCCTGCCCCGGTTA ATTTGCATATAATATTTCCTAGTAACTATAGAGGCTTAATGTGCGATAAAAGACA GATAATCTGTTCTTTTTAATACTAGCTACATTTTACATGATAGGCTTGGATTTCTA TAAGAGATACAAATACTAAATTATTATTTTAAAAAACAGCACAAAAGGAAACTC ACCCTAACTGTAAAGTAATTGTGTGTTTTGAGACTATAAATATCCCTTGGAGAAA AGCCTTGTTTG (SEQ ID NO: 308). In some cases, a human U7 promoter can comprise a sequence with at least about: 70%, 75%, 80%, 85%, 90%, 95%, or 99% sequence identity to: TTAACAACAACGAAGGGGCTGTGACTGGCTGCTTTCTCAACCAATCAGCACCGA ACTCATTTGCATGGGCTGAGAACAAATGTTCGCGAACTCTAGAAATGAATGACTT AAGTAAGTTCCTTAGAATATTATTTTTCCTACTGAAAGTTACCACATGCGTCGTTG TTTATACAGTAATAGGAACAAGAAAAAAGTCACCTAAGCTCACCCTCATCAATT GTGGAGTTCCTTTATATCCCATCTTCTCTCCAAACACATACGCA (SEQ ID NO: 309). In some cases, a mouse U7 promoter can comprise a sequence with at least about: 70%, 75%, 80%, 85%, 90%, 95%, or 99% sequence identity to: TTAACAACATAGGAGCTGTGATTGGCTGTTTTCAGCCAATCAGCACTGACTCATT TGCATAGCCTTTACAAGCGGTCACAAACTCAAGAAACGAGCGGTTTTAATAGTCT TTTAGAATATTGTTTATCGAACCGAATAAGGAACTGTGCTTTGTGATTCACATAT CAGTGGAGGGGTGTGGAAATGGCACCTTGATCTCACCCTCATCGAAAGTGGAGT TGATGTCCTTCCCTGGCTCGCTACAGACGCACTTCCGC (SEQ ID NO: 310). In some cases, a human U1 promoter can comprise a sequence with at least about: 70%, 75%, 80%, 85%, 90%, 95%, or 99% sequence identity to: TAAGGACCAGCTTCTTTGGGAGAGAACAGACGCAGGGGCGGGAGGGAAAAAGG GAGAGGCAGACGTCACTTCCTCTTGGCGACTCTGGCAGCAGATTGGTCGGTTGAG TGGCAGAAAGGCAGACGGGGACTGGGCAAGGCACTGTCGGTGACATCACGGAC AGGGCGACTTCTATGTAGATGAGGCAGCGCAGAGGCTGCTGCTTCGCCACTTGCT GCTTCGCCACGAAGGGAGTTCCCGTGCCCTGGGAGCGGGTTCAGGACCGCTGAT CGGAAGTGAGAATCCCAGCTGTGTGTCAGGGCTGGAAAGGGCTCGGGAGTGCGC GGGGCAAGTGACCGTGTGTGTAAAGAGTGAGGCGTATGAGGCTGTGTCGGGGCA GAGCCCGAAGATCTC (SEQ ID NO: 311). In some cases, a CMV promoter can comprise a sequence with at least about: 70%, 75%, 80%, 85%, 90%, 95%, or 99% sequence identity to:

(SEQ ID NO: 312)
ATACGCGTTGACATTGATTATTGACTAGTTATTAATAGTAATCAA
TTACGGGGTCATTAGTTCATAGCCCATATATGGAGTTCCGCGTTA
CATAACTTACGGTAAATGGCCCGCCTGGCTGACCGCCCAACGACC
CCCGCCCATTGACGTCAATAATGACGTATGTTCCCATAGTAACGC
CAATAGGGACTTTCCATTGACGTCAATGGGTGGAGTATTTACGGT
AAACTGCCCACTTGGCAGTACATCAAGTGTATCATATGCCAAGTA
CGCCCCCTATTGACGTCAATGACGGTAAATGGCCCGCCTGGCATT
ATGCCCAGTACATGACCTTATGGGACTTTCCTACTTGGCAGTACA
TCTACGTATTAGTCATCGCTATTACCATGGTGATGCGGTTTTGGC
AGTACATCAATGGGCGTGGATAGCGGTTTGACTCACGGGGATTTC
CAAGTCTCCACCCCATTGACGTCAATGGGAGTTTGTTTTGGCACC
AAAATCAACGGGACTTTCCAAAATGTCGTAACAACTCCGCCCCAT
TGACGCAAATGGGCGGTAGGCGTGTACGGTGGGAGGTCTATATAA
GCAGAGCTCGTTTAGTGAACCGTCAGATCGCCTGGAGACGCCATC
CACGCTGTTTTGACCTCCATAGAAGACACCGGGACCGATCCAGCC
TCCGGACTCTAGAGGATCGAACC.

### E. Chemically Modified Guide RNAs

An engineered guide RNA as described herein for use in treating a disease or condition in a subject can comprise at least one chemical modification. In some embodiments, the engineered guide RNA can comprise at least one, two, three, four, five, six, seven, eight, nine, ten, 11, 12, 13, 14, 15, 16, 17, 18, 19, 20, 30, 50, 100, or more chemical modifications. In some embodiments, the engineered guide RNA described herein may not comprise a chemical modification. In some cases, the engineered guide RNAs disclosed herein with barbell macro-footprints can be manufactured, chemically modified, and delivered directly to a subject in need thereof as RNA (without a vector, such as an AAV).

Exemplary chemical modifications comprise any one of: 5′ adenylate, 5′ guanosine-triphosphate cap, 5′ N7-Methylguanosine-triphosphate cap, 5′ triphosphate cap, 3′ phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphothioate DNA, phosphorothioate RNA, UNA, pseudouridine-5′-triphosphate, 5-methylcytidine-5′-triphosphate, 2-O-methyl 3-phosphorothioate, or any combinations thereof.

A chemical modification can be made at any location of the engineered guide RNA. In some cases, a modification may be located in a 5′ or 3′ end, or both. In some cases, a polynucleotide can comprise a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. In some cases, more than one modification can be made to the engineered guide RNA. In some cases, a modification can be permanent. In other cases, a modification can be transient. In some cases, multiple modifications may be made to the engineered guide RNA. The engineered guide RNA modification can alter physio-chemical properties of a nucleotide, such as their conformation, polarity, hydrophobicity, chemical reactivity, base-pairing interactions, or any combination thereof.

In some embodiments, a chemical modification can also be a phosphorothioate substitute. In some cases, a natural phosphodiester bond can be susceptible to rapid degradation by cellular nucleases and a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases may be of high probability in-vivo or in-vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

In some embodiments, a chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, a chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered guide RNA comprises at least one chemically modified base. In some instances, an engineered guide RNA can comprise 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, a chemical modification of the engineered guide RNA can comprise a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Chemical modifications to the engineered guide RNA include any modification contained herein, while some exemplary modifications are recited in Table 3.

TABLE 3
Exemplary Chemical Modification

| Modification of | |
|---|---|
| engineered guide RNA | Examples |
| Modification of one or both | sulfur (S), selenium (Se), BR3 (wherein R can be, e.g., hydrogen, |
| of the non-linking | alkyl, or aryl), C (e.g., an alkyl group, an aryl group, and the like), |
| phosphate oxygens in the | H, NR2, wherein R can be, e.g., hydrogen, alkyl, or aryl, or |
| phosphodiester backbone | wherein R can be, e.g., alkyl or aryl |
| linkage | |
| Modification of one or more | sulfur (S), selenium (Se), BR3 (wherein R can be, e.g., hydrogen, |
| of the linking phosphate | alkyl, or aryl), C (e.g., an alkyl group, an aryl group, and the like), |
| oxygens in the | H, NR2, wherein R can be, e.g., hydrogen, alkyl, or aryl, or |
| phosphodiester backbone | wherein R can be, e.g., alkyl or aryl |
| linkage | |
| Replacement of the | methyl phosphonate, hydroxylamino, siloxane, carbonate, |
| phosphate moiety with | carboxymethyl, carbamate, amide, thioether, ethylene oxide linker, |
| “dephospho” linkers | sulfonate, sulfonamide, thioformacetal, formacetal, oxime, |
| | methyleneimino, methylenemethylimino, methylenehydrazo, |
| | methylenedimethylhydrazo, or methyleneoxymethylimino |
| Modification or replacement | Nucleic acid analog (examples of nucleotide analogs can be found |
| of a naturally occurring | in PCT/US2015/025175, PCT/US2014/050423, |
| nucleobase | PCT/US2016/067353, PCT/US2018/041503, |
| | PCT/US2018/041509, PCT/US2004/011786, or |
| | PCT/US2004/011833, all of which are expressly incorporated by |
| | reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, alkyl or |
| | aryl phosphonates, phosphonoacetate, or phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, or |
| | phosphotriesters |
| Substitution of the | methyl phosphonate, hydroxylamino, siloxane, carbonate, |
| phosphate group | carboxymethyl, carbamate, amide, thioether, ethylene oxide linker, |
| | sulfonate, sulfonamide, thioformacetal, formacetal, oxime, |
| | methyleneimino, methylenemethylimino, methylenehydrazo, |
| | methylenedimethylhydrazo, or methyleneoxymethylimino. |
| Modification of the | morpholino, cyclobutyl, pyrrolidine, or peptide nucleic acid (PNA) |
| ribophosphate backbone | nucleoside surrogates |
| Modifications to the sugar | Locked nucleic acid (LNA), unlocked nucleic acid (UNA), or |
| of a nucleotide | bridged nucleic acid (BNA) |
| Modification of a | 2′-O-methyl, 2′-O-methoxy-ethyl (2′-MOE), 2′-fluoro, 2′- |
| constituent of the ribose | aminoethyl, 2′-deoxy-2′-fuloarabinou-cleic acid, 2′-deoxy, 2′-O- |
| sugar | methyl, 3′-phosphorothioate, 3′-phosphonoacetate (PACE), or 3′- |
| | phosphonothioacetate (thioPACE) |
| Modifications to the base of | Modification of A, T, C, G, or U |
| a nucleotide | |
| Stereopure of nucleotide | S conformation of phosphorothioate or R conformation of |
| | phosphorothioate |

### Modification of Phosphate Backbone

In some embodiments, the chemical modification can comprise modification of one or both of the non-linking phosphate oxygens in the phosphodiester backbone linkage or modification of one or more of the linking phosphate oxygens in the phosphodiester backbone linkage. As used herein, “alkyl” may be meant to refer to a saturated hydrocarbon group which may be straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” may refer to monocyclic or polycyclic (e.g. having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” may refer to an aliphatic group containing at least one double bond. As used herein, “alkynyl” may refer to a straight or branched hydrocarbon chain containing 2-12 carbon atoms and characterized in having one or more triple bonds. Examples of alkynyl groups can include ethynyl, propargyl, or 3-hexynyl. “Arylalkyl”

or “aralkyl” may refer to an alkyl moiety in which an alkyl hydrogen atom may be replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” may refer to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” may refer to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” may refer to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way may be a stereogenic center. The stereogenic phosphorous atom can possess either the “R” configuration (herein Rp) or the “S” configuration (herein Sp). In some cases, the engineered guide RNA can comprise stereopure nucleotides comprising S conformation of phosphorothioate or R conformation of phosphorothioate. In some embodiments, the chiral phosphate product may be present in a diastereomeric excess of 50%, 60%, 70%, 80%, 90%, or more. In some embodiments, the chiral phosphate product may be present in a diastereomeric excess of 95%. In some embodiments, the chiral phosphate product may be present in a diastereomeric excess of 96%. In some embodiments, the chiral phosphate product may be present in a diastereomeric excess of 97%. In some embodiments, the chiral phosphate product may be present in a diastereomeric excess of 98%. In some embodiments, the chiral phosphate product may be present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). In some cases, the replacement can occur at either or both of the linking oxygens.

In certain embodiments, nucleic acids comprise linked nucleic acids. Nucleic acids can be linked together using any inter nucleic acid linkage. The two main classes of inter nucleic acid linking groups are defined by the presence or absence of a phosphorus atom. Representative phosphorus containing inter nucleic acid linkages include, but are not limited to, phosphodiesters, phosphotriesters, methylphosphonates, phosphoramidate, and phosphorothioates (P=S). Representative non-phosphorus containing inter nucleic acid linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N*-dimethylhydrazine (—CH₂—N(CH₃)—N(CH₃)). In certain embodiments, inter nucleic acids linkages having a chiral atom can be prepared as a racemic mixture, as separate enantiomers, e.g., alkylphosphonates and phosphorothioates. Unnatural nucleic acids can contain a single modification. Unnatural nucleic acids can contain multiple modifications within one of the moieties or between different moieties.

In some cases, backbone phosphate modifications to nucleic acid include, but are not limited to, methyl phosphonate, phosphorothioate, phosphoramidate (bridging or non-bridging), phosphotriester, phosphorodithioate, phosphodithioate, and boranophosphate, and can be used in any combination. Other non-phosphate linkages may also be used.

In some embodiments, backbone modifications (e.g., methylphosphonate, phosphorothioate, phosphoroamidate and phosphorodithioate internucleotide linkages) can confer immunomodulatory activity on the modified nucleic acid and/or enhance their stability in-vivo.

In some instances, a phosphorous derivative (or modified phosphate group) may be attached to the sugar or sugar analog moiety in and can be a monophosphate, diphosphate, triphosphate, alkylphosphonate, phosphorothioate, phosphorodithioate, phosphoramidate or the like.

In some cases, backbone modification comprises replacing the phosphodiester linkage with an alternative moiety such as an anionic, neutral or cationic group. Examples of such modifications include: anionic internucleoside linkage; N3′ to P5′ phosphoramidate modification; boranophosphate DNA; prooligonucleotides; neutral internucleoside linkages such as methylphosphonates; amide linked DNA; methylene(methylimino) linkages; formacetal and thioformacetal linkages; backbones containing sulfonyl groups; morpholino oligos; peptide nucleic acids (PNA); and positively charged deoxyribonucleic guanidine (DNG) oligos. A modified nucleic acid may comprise a chimeric or mixed backbone comprising one or more modifications, e.g., a combination of phosphate linkages such as a combination of phosphodiester and phosphorothioate linkages.

In some cases, substitutes for the phosphate include, for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts. It may be also understood in a nucleotide substitute that both the sugar and the phosphate moieties of the nucleotide can be replaced, by for example an amide type linkage (aminoethylglycine) (PNA). It may be also possible to link other types of molecules (conjugates) to nucleotides or nucleotide analogs to enhance for example, cellular uptake. In some cases, conjugates can be chemically linked to the nucleotide or nucleotide analogs. Such conjugates include but are not limited to lipid moieties such as a cholesterol moiety, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethylammonium 1-di-O-hexadecyl-rac-glycero-S—H-phosphonate, a polyamine or a polyethylene glycol chain, or adamantane acetic acid, a palmityl moiety, or an octadecylamine or hexylamino-carbonyl-oxycholesterol moiety.

In some embodiments, a chemical modification described herein can comprise modification of a phosphate backbone. In some embodiments, the engineered guide RNA described herein can comprise at least one chemically modified phosphate backbone. Exemplary chemically modification of the phosphate group or backbone can include replacing one or more of the oxygens with a different substituent. Furthermore, the modified nucleotide present in the engineered guide RNA can include the replacement of an unmodified phosphate moiety with a modified phosphate as described herein. In some embodiments, the modification of the phosphate backbone can include alterations resulting in either an uncharged linker or a charged linker with unsymmetrical charge distribution. Exemplary modified phosphate groups can include, phosphorothioate, phosphonothioacetate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR3 (wherein R can be, e.g., hydrogen, alkyl, or aryl), C (e.g., an alkyl group, an aryl group, and the like), H, NR2 (wherein R can be, e.g., hydrogen, alkyl, or aryl), or OR (wherein R can be, e.g., alkyl or aryl). The phosphorous atom in an unmodified phosphate group may be achiral. However, replacement of one of the non-bridging oxygens with one of the above atoms or groups of atoms can render the phosphorous atom chiral; that may be to say that a phosphorous atom in a phosphate group modified in this way may be a stereogenic center. The stereogenic phosphorous atom can possess either the “R” configuration (herein Rp) or the “S” configuration (herein Sp). In such case, the chemically modified engineered guide RNA can be stereopure (e.g., S or R confirmation). In some cases, a chemically modified engineered guide RNA comprises stereopure phosphate modification. For example, the chemically modified engineered guide RNA can comprise S conformation of phosphorothioate or R conformation of phosphorothioate.

Phosphorodithioates have both non-bridging oxygens replaced by sulfur. The phosphorus center in the phosphorodithioates may be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl).

In some cases, the phosphate linker can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

### Replacement of Phosphate Moiety

In some embodiments, at least one phosphate group of the engineered guide RNA can be chemically modified. In some embodiments, the phosphate group can be replaced by non-phosphorus containing connectors. In some embodiments, the phosphate moiety can be replaced by dephospho linker. In some embodiments, the charge phosphate group can be replaced by a neutral group. In some cases, the phosphate group can be replaced by methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In some embodiments, nucleotide analogs described herein can also be modified at the phosphate group. Modified phosphate group can include modification at the linkage between two nucleotides with phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates (e.g., 3′-amino phosphoramidate and aminoalkylphosphoramidates), thionophosphoramidates, thionoalkylphosphonates, thionoalkylphosphotriesters, and boranophosphates. In some cases, the phosphate or modified phosphate linkage between two nucleotides can be through a 3′-5′ linkage or a 2′-5′ linkage, and the linkage contains inverted polarity such as 3′-5′ to 5′-3′ or 2′-5′ to 5′-2′.

### Substitution of Phosphate Group

In some embodiments, a chemical modification described herein can comprise modification by replacement of a phosphate group. In some embodiments, the engineered guide RNA described herein can comprise at least one chemically modification comprising a phosphate group substitution or replacement. Exemplary phosphate group replacement can include non-phosphorus containing connectors. In some embodiments, the phosphate group substitution or replacement can include replacing charged phosphate group can by a neutral moiety. Exemplary moieties which can replace the phosphate group can include methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

### Modification of the Ribophosphate Backbone

In some embodiments, the chemical modification described herein can comprise modifying ribophosphate backbone of the engineered guide RNA. In some embodiments, the engineered guide RNA described herein can comprise at least one chemically modified ribophosphate backbone. Exemplary chemically modified ribophosphate backbone can include scaffolds that can mimic nucleic acids can also be constructed wherein the phosphate linker and ribose sugar may be replaced by nuclease resistant nucleoside or nucleotide surrogates. In some embodiments, the nucleobases can be tethered by a surrogate backbone. Examples can include morpholino, cyclobutyl, pyrrolidine and peptide nucleic acid (PNA) nucleoside surrogates.

### Modification of Sugar

In some embodiments, the chemical modification described herein can comprise modifying of sugar. In some embodiments, the engineered guide RNA described herein can comprise at least one chemically modified sugar. Exemplary chemically modified sugar can include 2′ hydroxyl group (OH) modified or replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion. The 2′-alkoxide can catalyze degradation by intramolecular nucleophilic attack on the linker phosphorus atom. Examples of “oxy”-2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR, wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the “oxy”-2′ hydroxyl group modification can include (LNA, in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). In some embodiments, the “oxy”-2′ hydroxyl group modification can include the methoxyethyl group (MOE), (OCH₂CH₂OCH₃, e.g., a PEG derivative). In some cases, the deoxy modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which can be optionally substituted with e.g., an amino as described herein. In some instances, the sugar group can also contain one or more carbons that possess the opposite stereochemical configuration than that of the corresponding carbon in ribose. Thus, a modified nucleic acid can include nucleotides containing e.g., arabinose, as the sugar. The nucleotide “monomer” can have an alpha linkage at the F position on the sugar, e.g., alpha-nucleosides. The modified nucleic acids can also include “abasic” sugars, which lack a nucleobase at C—. The abasic sugars can also be further modified at one or more of the constituent sugar atoms. The modified nucleic acids can also include one or more sugars that may be in the L form, e.g., L-nucleosides. In some aspects, the engineered guide RNA described herein includes the sugar group ribose, which may be a 5-membered ring having an oxygen. Exemplary modified nucleosides and modified nucleotides can include replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). In some embodiments, the modified nucleotides can include multicyclic forms (e.g., tricyclo; and “unlocked” forms, such as glycol nucleic acid (GNA) (e.g., R-GNA or S-GNA, where ribose may be replaced by glycol units attached to phosphodiester bonds), threose nucleic acid. In some embodiments, the modifications to the sugar of the engineered guide RNA comprises modifying the engineered guide RNA to include locked nucleic acid (LNA), unlocked nucleic acid (UNA), or bridged nucleic acid (BNA).

### Modification of a Constituent of the Ribose Sugar

In some embodiments, the engineered guide RNA described herein can comprise at least one chemical modification of a constituent of the ribose sugar. In some embodiments, the chemical modification of the constituent of the ribose sugar can include 2′-O-methyl, 2′-O-methoxy-ethyl (2′-MOE), 2′-fluoro, 2′-aminoethyl, 2′-deoxy-2′-fuloarabinou-cleic acid, 2′-deoxy, 2′-O-methyl, 3′-phosphorothioate, 3′-phosphonoacetate (PACE), or 3′-phosphonothioacetate (thioPACE). In some embodiments, the chemical modification of the constituent of the ribose sugar comprises unnatural nucleic acid. In some instances, the unnatural nucleic acids include modifications at the 5′-position and the 2′-position of the sugar ring, such as 5′-CH₂-substituted 2′-O-protected nucleosides. In some cases, unnatural nucleic acids include amide linked nucleoside dimers that can be prepared for incorporation into oligonucleotides. In some cases, the 3′ linked nucleoside in the dimer (5′ to 3′) comprises a 2′-OCH₃ and a 5′-(S)—CH₃. Unnatural nucleic acids can include 2′-substituted 5′-CH₂ (or O) modified nucleosides. Unnatural nucleic acids can include 5′-methylenephosphonate DNA and RNA monomers, and dimers. Unnatural nucleic acids can include 5′-phosphonate monomers having a 2′-substitution and other modified 5′-phosphonate monomers. Unnatural nucleic acids can include 5′-modified methylenephosphonate monomers. Unnatural nucleic acids can include analogs of 5′ or 6′-phosphonate ribonucleosides comprising a hydroxyl group at the 5′ and/or 6′-position. Unnatural nucleic acids can include 5′-phosphonate deoxyribonucleoside monomers and dimers having a 5′-phosphate group. Unnatural nucleic acids can include nucleosides having a 6′-phosphonate group wherein the 5′ or/and 6′-position may be unsubstituted or substituted with a thio-tert-butyl group (SC(CH₃)₃) (and analogs thereof); a methyleneamino group (CH₂NH₂) (and analogs thereof) or a cyano group (CN) (and analogs thereof).

In some embodiments, unnatural nucleic acids also include modifications of the sugar moiety. In some cases, nucleic acids can contain one or more nucleosides wherein the sugar group has been modified. Such sugar modified nucleosides may impart enhanced nuclease stability, increased binding affinity, or some other beneficial biological property. In certain embodiments, nucleic acids can comprise a chemically modified ribofuranose ring moiety. Examples of chemically modified ribofuranose rings include, without limitation, addition of substituent groups (including 5′ and/or 2′ substituent groups; bridging of two ring atoms to form bicyclic nucleic acids; replacement of the ribosyl ring oxygen atom with S, N(R), or C(R₁)(R₂) (R=H, C₁-C₁₂ alkyl or a protecting group); and combinations thereof.

In some instances, the engineered guide RNA described herein can comprise modified sugars or sugar analogs. Thus, in addition to ribose and deoxyribose, the sugar moiety can be pentose, deoxypentose, hexose, deoxyhexose, glucose, arabinose, xylose, lyxose, or a sugar “analog” cyclopentyl group. The sugar can be in a pyranosyl or furanosyl form. The sugar moiety can be the furanoside of ribose, deoxyribose, arabinose or 2′-O-alkylribose, and the sugar can be attached to the respective heterocyclic bases either in [alpha] or [beta] anomeric configuration. Sugar modifications include, but are not limited to, 2′-alkoxy-RNA analogs, 2′-amino-RNA analogs, 2′-fluoro-DNA, and 2′-alkoxy- or amino-RNA/DNA chimeras. For example, a sugar modification may include 2′-O-methyl-uridine or 2′-O-methyl-cytidine. Sugar modifications include 2′-O-alkyl-substituted deoxyribonucleosides and 2′-O-ethyleneglycol-like ribonucleosides.

In some cases, modifications to the sugar moiety include natural modifications of the ribose and deoxy ribose as well as unnatural modifications. Sugar modifications include, but are not limited to, the following modifications at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀, alkyl or C₂ to C₁₀ alkenyl and alkynyl. 2′ sugar modifications also include but are not limited to —O[(CH₂)ₙO]ₘ·CH₃, —O(CH₂)ₙOCH₃, —O(CH₂)ₙNH₂, —O(CH₂)ₙCH₃, —O(CH₂)ₙONH₂, and —O(CH₂)ₙON[(CH₂)ₙ CH₃)]₂, where n and m may be from 1 to about 10. Other chemical modifications at the 2′ position include but are not limited to: C₁ to C₁₀ lower alkyl, substituted lower alkyl, alkaryl, aralkyl, O-alkaryl, O-aralkyl, SH, SCH₃, OCN, Cl, Br, CN, CF₃, OCF₃, SOCH₃, SO₂ CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of the 5′ terminal nucleotide. Chemically modified sugars also include those that contain modifications at the bridging ring oxygen, such as CH₂ and S. Nucleotide sugar analogs can also have sugar mimetics such as cyclobutyl moieties in place of the pentofuranosyl sugar. Examples of nucleic acids having modified sugar moieties include, without limitation, nucleic acids comprising 5′-vinyl, 5′-methyl (R or S), 4′-S, 2′-F, 2′-OCH₃, and 2′-O(CH₂)₂OCH₃ substituent groups. The substituent at the 2′ position can also be selected from allyl, amino, azido, thio, O-allyl, O—(C₁-C₁₀ alkyl), OCF₃, O(CH₂)₂SCH₃, O(CH₂)₂—O—N(Rₘ)(Rₙ), and O—CH₂—C(═O)—N(Rₘ)(Rₙ), where each Rₘ and Rₙ is, independently, H or substituted or unsubstituted C₁-C₁₀ alkyl.

In certain embodiments, nucleic acids described herein can include one or more bicyclic nucleic acids. In certain such embodiments, the bicyclic nucleic acid comprises a bridge between the 4′ and the 2′ ribosyl ring atoms. In certain embodiments, nucleic acids provided herein can include one or more bicyclic nucleic acids wherein the bridge comprises a 4′ to 2′ bicyclic nucleic acid. Examples of such 4′ to 2′ bicyclic nucleic acids include, but are not limited to, one of the formulae: 4′-(CH₂)—O-2′ (LNA); 4′-(CH₂)—S-2′; 4′-(CH₂)₂—O-2′ (ENA); 4′-CH(CH₃)—O-2′ and 4′-CH(CH₂OCH₃)—O-2′, and analogs thereof; 4′-C(CH₃)(CH₃)—O-2′ and analogs thereof.

### Modifications on the Base of Nucleotide

In some embodiments, the chemical modification described herein can comprise modification of the base of nucleotide (e.g., the nucleobase). Exemplary nucleobases can include adenine (A), thymine (T), guanine (G), cytosine (C), and uracil (U). These nucleobases can be modified or replaced to in the engineered guide RNA described herein. The nucleobase of the nucleotide can be independently selected from a purine, a pyrimidine, a purine or pyrimidine analog. In some embodiments, the nucleobase can be naturally-occurring or synthetic derivatives of a base.

In some embodiments, the chemical modification described herein can comprise modifying an uracil. In some embodiments, the engineered guide RNA described herein can comprise at least one chemically modified uracil. Exemplary chemically modified uracil can include pseudouridine, pyridin-4-one ribonucleoside, 5-aza-uridine, 6-aza-uridine, 2-thio-5-aza-uridine, 2-thio-uridine, 4-thio-uridine, 4-thio-pseudouridine, 2-thio-pseudouridine, 5-hydroxy-uridine, 5-aminoallyl-uridine, 5-halo-uridine (e.g., 5-iodo-uridine or 5-bromo-uridine), 3-methyl-uridine, 5-methoxy-uridine, uridine 5-oxyacetic acid, uridine 5-oxyacetic acid methyl ester, 5-carboxymethyl-uridine, 1-carboxymethyl-pseudouridine, 5-carboxyhydroxymethyl-uridine, 5-carboxyhydroxymethyl-uridine methyl ester, 5-methoxycarbonylmethyl-uridine, 5-methoxycarbonylmethyl-2-thio-uridine, 5-aminomethyl-2-thio-uridine, 5-methylaminomethyl-uridine, 5-methylaminomethyl-2-thio-uridine, 5-methylaminomethyl-2-seleno-uridine, 5-carbamoylmethyl-uridine, 5-carboxymethylaminomethyl-uridine, 5-carboxymethylaminomethyl-2-thio-uridine, 5-propynyl-uridine, 1-propynyl-pseudouridine, 5-taurinomethyl-uridine, 1-taurinomethyl-pseudouridine, 5-taurinomethyl-2-thio-uridine, 1-taurinomethyl-4-thio-pseudouridine, 5-methyl-uridine, 1 methyl-pseudouridine, 5-methyl-2-thio-uridine, 1-methyl-4-thio-pseudouridine, 4-thio-1-methyl-pseudouridine, 3-methyl-pseudouridine, 2-thio-1-methyl-pseudouridine, 1-methyl-1-deaza-pseudouridine, 2-thio-1-methyl-1-deaza-pseudouridine, dihydroundine, dihydropseudoundine, 5,6-dihydrouridine, 5-methyl-dihydrouridine, 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine, 1-methyl-3-(3-amino-3-carboxypropy pseudouridine, 5-(isopentenylaminomethyl) uridine, 5-(isopentenylaminomethyl)-2-thio-uridine, α-thio-uridine, 2′-O-methyl-uridine, 5,2′-O-dimethyl-uridine, 2′-O-methyl-pseudouridine, 2-thio-2′-O-methyl-uridine, 5-methoxycarbonylmethyl-2′-O-methyl-uridine, 5-carbamoylmethyl-2′-O-methyl-uridine, 5-carboxymethylaminomethyl-2′-O-methyl-uridine, 3,2′-O-dimethyl-uridine, 5-(isopentenylaminomethyl)-2′-O-methyl-uridine, 1-thio-uridine, deoxythymidine, 2′-F-ara-uridine, 2′-F-uridine, 2′-OH-ara-uridine, 5-(2-carbomethoxyvinyl) uridine, 5-[3-(1-E-propenylamino)uridine, pyrazolo[3,4-d]pyrimidines, xanthine, and hypoxanthine.

In some embodiments, the chemical modification described herein can comprise modifying a cytosine. In some embodiments, the engineered guide RNA described herein can comprise at least one chemically modified cytosine. Exemplary chemically modified cytosine can include 5-aza-cytidine, 6-aza-cytidine, pseudoisocytidine, 3-methyl-cytidine, N4-acetyl-cytidine, 5-formyl-cytidine, N4-methyl-cytidine, 5-methyl-cytidine, 5-halo-cytidine, 5-hydroxymethyl-cytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine, α-thio-cytidine, 2′-O-methyl-cytidine, 5,2′-O-dimethyl-cytidine, N4-acetyl-2′-O-methyl-cytidine, N4,2′-O-dimethyl-cytidine, 5-formyl-2′-O-methyl-cytidine, N4,N4,2′-O-trimethyl-cytidine, 1-thio-cytidine, 2′-F-ara-cytidine, 2′-F-cytidine, and 2′-OH-ara-cytidine.

In some embodiments, the chemical modification described herein can comprise modifying an adenine. In some embodiments, the engineered guide RNA described herein can comprise at least one chemically modified adenine. Exemplary chemically modified adenine can include 2-amino-purine, 2,6-diaminopurine, 2-amino-6-halo-purine (e.g., 2-amino-6-chloro-purine), 6-halo-purine (e.g., 6-chloi-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine, 2-methyl-adenine, N6-methyl-adenosine, 2-methylthio-N6-methyl-adenosine, N6-isopentenyl-adenosine, 2-methylthio-N6-isopentenyl-adenosine, N6-(cis-hydroxyisopentenyl) adenosine, 2-methylthio-N6-(cis-hydroxyisopentenyl) adenosine, N6-glycinylcarbamoyl-adenosine, N6-threonylcarbamoyl-adenosine, N6-methyl-N6-threonylcarbamoyl-adenosine, 2-methylthio-N6-threonylcarbamoyl-adenosine, N₆, N₆-dimethyl-adenosine, N₆-hydroxynorvalylcarbamoyl-adenosine, 2-methylthio-N₆-hydroxynorvalylcarbamoyl-adenosine, N₆-acetyl-adenosine, 7-methyl-adenine, 2-methylthio-adenine, 2-methoxy-adenine, α-thio-adenosine, 2′-O-methyl-adenosine, N_{6, 2}′-O-dimethyl-adenosine, N₆-Methyl-2′-deoxyadenosine, N₆, N_{6, 2}′-O-trimethyl-adenosine, 1,2′-O-dimethyl-adenosine, 2′-O-ribosyladenosine (phosphate) (Ar(p)), 2-amino-N₆-methyl-purine, 1-thio-adenosine, 8-azido-adenosine, 2′-F-ara-adenosine, 2′-F-adenosine, 2′-OH-ara-adenosine, and N₆-(19-amino-pentaoxanonadecyl)-adenosine.

In some embodiments, the chemical modification described herein can comprise modifying a guanine. In some embodiments, the engineered guide RNA described herein can comprise at least one chemically modified guanine. Exemplary chemically modified guanine can include inosine, 1-methyl-inosine, wyosine, methylwyosine, 4-demethyl-wyosine, isowyosine, wybutosine, peroxywybutosine, hydroxywybutosine, undermodified hydroxywybutosine, 7-deaza-guanosine, queuosine, epoxyqueuosine, galactosyl-queuosine, mannosyl-queuosine, 7-cyano-7-deaza-guanosine, 7-aminomethyl-7-deaza-guanosine, archaeosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methyl-inosine, 6-methoxy-guanosine, 1-methyl-guanosine, N2-methyl-guanosine, N2, N2-dimethyl-guanosine, N2, 7-dimethyl-guanosine, N2, N2, 7-dimethyl-guanosine, 8-oxo-guanosine, 7-methyl-8-oxo-guanosine, 1-meththio-guanosine, N2-methyl-6-thio-guanosine, N2,N2-dimethyl-6-thio-guanosine, α-thio-guanosine, 2′-O-methyl-guanosine, N2-methyl-2′-O-methyl-guanosine, N2,N2-dimethyl-2′-O-methyl-guanosine, 1-methyl-2′-O-methyl-guanosine, N2, 7-dimethyl-2′-O-methyl-guanosine, 2′-O-methyl-inosine, 1, 2′-O-dimethyl-inosine, 6-O-phenyl-2′-deoxyinosine, 2′-O-ribosylguanosine, 1-thio-guanosine, 6-O-methyguanosine, 0(6)-Methyl-2′-deoxyguanosine, 2′-F-ara-guanosine, and 2′-F-guanosine.

In some cases, the chemical modification of the engineered guide RNA can include introducing or substituting a nucleic acid analog or an unnatural nucleic acid into the engineered guide RNA. In some embodiments, nucleic acid analog can be any one of the chemically modified nucleic acid described herein. Exemplary nucleic acid analogs can be found in PCT/US2021/034272, PCT/US2015/025175, PCT/US2014/050423, PCT/US2016/067353, PCT/US2018/041503, PCT/US18/041509, PCT/US2004/011786, or PCT/US2004/011833, all of which are expressly incorporated by reference in their entireties. In some cases, the chemically modified nucleotide described herein can include a variant of guanosine, uridine, adenosine, thymidine, and cytosine, including any natively occurring or non-natively occurring guanosine, uridine, adenosine, thymidine or cytidine that has been altered chemically, for example by acetylation, methylation, hydroxylation. Exemplary chemically modified nucleotide can include 1-methyl-adenosine, 1-methyl-guanosine, 1-methyl-inosine, 2,2-dimethyl-guanosine, 2,6-diaminopurine, 2′-amino-2′-deoxyadenosine, 2′-amino-2′-deoxycytidine, 2′-amino-2′-deoxyguanosine, 2′-amino-2′-deoxyuridine, 2-amino-6-chloropurineriboside, 2-aminopurine-riboside, 2′-araadenosine, 2′-aracytidine, 2′-arauridine, 2′-azido-2′-deoxyadenosine, 2′-azido-2′-deoxycytidine, 2′-azido-2′-deoxyguanosine, 2′-azido-2′-deoxyuridine, 2-chloroadenosine, 2′-fluoro-2′-deoxyadenosine, 2′-fluoro-2′-deoxycytidine, 2′-fluoro-2′-deoxyguanosine, 2′-fluoro-2′-deoxyuridine, 2′-fluorothymidine, 2-methyl-adenosine, 2-methyl-guanosine, 2-methyl-thio-N6-isopenenyl-adenosine, 2′-O-methyl-2-aminoadenosine, 2′-O-methyl-2′-deoxyadenosine, 2′-O-methyl-2′-deoxycytidine, 2′-O-methyl-2′-deoxyguanosine, 2, —O-methyl-2′-deoxyuridine, 2′-O-methyl-5-methyluridine, 2′-O-methylinosine, 2′-O-methylpseudouridine, 2-thiocytidine, 2-thio-cytidine, 3-methyl-cytidine, 4-acetyl-cytidine, 4-thiouridine, 5-(carboxyhydroxymethyl)-uridine, 5,6-dihydrouridine, 5-aminoallylcytidine, 5-aminoallyl-deoxyuridine, 5-bromouridine, 5-carboxymethylaminomethyl-2-thio-uracil, 5-carboxymethylamonomethyl-uracil, 5-chloro-ara-cytosine, 5-fluoro-uridine, 5-iodouridine, 5-methoxycarbonylmethyl-uridine, 5-methoxy-uridine, 5-methyl-2-thio-uridine, 6-Azacytidine, 6-azauridine, 6-chloro-7-deaza-guanosine, 6-chloropurineriboside, 6-mercapto-guanosine, 6-methyl-mercaptopurine-riboside, 7-deaza-2′-deoxy-guanosine, 7-deazaadenosine, 7-methyl-guanosine, 8-azaadenosine, 8-bromo-adenosine, 8-bromo-guanosine, 8-mercapto-guanosine, 8-oxoguanosine, benzimidazole-riboside, beta-D-mannosyl-queosine, dihydro-uridine, inosine, N1-methyladenosine, N6-([6-aminohexyl]carbamoylmethyl)-adenosine, N6-isopentenyl-adenosine, N6-methyl-adenosine, N7-methyl-xanthosine, N-uracil-5-oxyacetic acid methyl ester, puromycin, queosine, uracil-5-oxyacetic acid, uracil-5-oxyacetic acid methyl ester, wybutoxosine, xanthosine, and xylo-adenosine. In some embodiments, the chemically modified nucleic acid as described herein comprises at least one chemically modified nucleotide selected from 2-amino-6-chloropurineriboside-5′-triphosphate, 2-aminopurine-riboside-5′-triphosphate, 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl-inosine-5′-triphosphate, 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 6-methylguanosine-5′-triphosphate, pseudouridine-5′-triphosphate, puromycin-5′-triphosphate, or xanthosine-5′-triphosphate. In some embodiments, the chemically modified nucleic acid as described herein can comprise at least one chemically modified nucleotide selected from pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the artificial nucleic acid as described herein comprises at least one chemically modified nucleotide selected from 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-I-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the chemically modified nucleic acid as described herein comprises at least one chemically modified nucleotide selected from 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In other embodiments, the chemically modified nucleic acid as described herein can comprise at least one chemically modified nucleotide selected from inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine. In certain embodiments, the chemically modified nucleic acid as described herein can comprise at least one chemically modified nucleotide selected from 6-aza-cytidine, 2-thio-cytidine, alpha-thio-cytidine, pseudo-iso-cytidine, 5-aminoallyl-uridine, 5-iodo-uridine, N1-methyl-pseudouridine, 5,6-dihydrouridine, alpha-thio-uridine, 4-thio-uridine, 6-aza-uridine, 5-hydroxy-uridine, deoxy-thymidine, 5-methyl-uridine, pyrrolo-cytidine, inosine, alpha-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, alpha-thio-adenosine, 8-azido-adenosine, 7-deaza-adenosine.

In some embodiments, a modified base of a unnatural nucleic acid includes, but may be not limited to, uracil-5-yl, hypoxanthin-9-yl (I), 2-aminoadenin-9-yl, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain unnatural nucleic acids, such as 5-substituted pyrimidines, 6-azapyrimidines and N-2 substituted purines, N-6 substituted purines, 0-6 substituted purines, 2-aminopropyladenine, 5-propynyluracil, 5-propynylcytosine, 5-methylcytosine, those that increase the stability of duplex formation, universal nucleic acids, hydrophobic nucleic acids, promiscuous nucleic acids, size-expanded nucleic acids, fluorinated nucleic acids, 5-substituted pyrimidines, 6-azapyrimidines and N-2, N-6 and 0-6 substituted purines, including 2-aminopropyladenine, 5-propynyluracil and 5-propynylcytosine. 5-methylcytosine (5-me-C), 5-hydroxymethyl cytosine, xanthine, hypoxanthine, 2-aminoadenine, 6-methyl, other alkyl derivatives of adenine and guanine, 2-propyl and other alkyl derivatives of adenine and guanine, 2-thiouracil, 2-thiothymine and 2-thiocytosine, 5-halouracil, 5-halocytosine, 5-propynyl (—C—C—CH₃) uracil, 5-propynyl cytosine, other alkynyl derivatives of pyrimidine nucleic acids, 6-azo uracil, 6-azo cytosine, 6-azo thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl, other 5-substituted uracils and cytosines, 7-methylguanine, 7-methyladenine, 2-F-adenine, 2-amino-adenine, 8-azaguanine, 8-azaadenine, 7-deazaguanine, 7-deazaadenine, 3-deazaguanine, 3-deazaadenine, tricyclic pyrimidines, phenoxazine cytidine([5,4-b][1,4]benzoxazin-2(3H)-one), phenothiazine cytidine (1H-pyrimido[5,4-b][1,4]benzothiazin-2(3H)-one), G-clamps, phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one), those in which the purine or pyrimidine base may be replaced with other heterocycles, 7-deaza-adenine, 7-deazaguanosine, 2-aminopyridine, 2-pyridone, azacytosine, 5-bromocytosine, bromouracil, 5-chlorocytosine, chlorinated cytosine, cyclocytosine, cytosine arabinoside, 5-fluorocytosine, fluoropyrimidine, fluorouracil, 5,6-dihydrocytosine, 5-iodocytosine, hydroxyurea, iodouracil, 5-nitrocytosine, 5-bromouracil, 5-chlorouracil, 5-fluorouracil, and 5-iodouracil, 2-amino-adenine, 6-thio-guanine, 2-thio-thymine, 4-thio-thymine, 5-propynyl-uracil, 4-thio-uracil, N4-ethylcytosine, 7-deazaguanine, 7-deaza-8-azaguanine, 5-hydroxycytosine, 2′-deoxyuridine, or 2-amino-2′-deoxyadenosine.

In some cases, the at least one chemical modification can comprise chemically modifying the 5′ or 3′ end such as 5′ cap or 3′ tail of the engineered guide RNA. In some embodiments, the engineered guide RNA can comprise a chemical modification comprising 3′ nucleotides which can be stabilized against degradation, e.g., by incorporating one or more of the modified nucleotides described herein. In this embodiment, uridines can be replaced with modified uridines, e.g., 5-(2-amino) propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein. In some embodiments, deaza nucleotides, e.g., 7-deaza-adenosine, can be incorporated into the gRNA. In some embodiments, O- and N-alkylated nucleotides, e.g., N6-methyladenosine, can be incorporated into the gRNA. In some embodiments, sugar-modified ribonucleotides can be incorporated, e.g., wherein the 2′ OH-group may be replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In some embodiments, the phosphate backbone can be modified as described herein, e.g., with a phosphothioate group. In some embodiments, the nucleotides in the overhang region of the gRNA can each independently be a modified or unmodified nucleotide including, but not limited to 2′-sugar modified, such as, 2-F 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), or any combinations thereof.

### Targets and Methods of Treatment

The present disclosure provides for compositions of engineered guide RNAs or engineered polynucleotides encoding guide RNAs and methods of use thereof, such as methods of treatment. In some embodiments, the engineered polynucleotides of the present disclosure encode for guide RNAs targeting a coding sequence of an RNA (e.g., a TIS).

The present disclosure provides for engineered guide RNAs that facilitate SNCA RNA editing when contacted with SNCA RNA to knockdown or reduce expression of alpha-synuclein protein. Knockdown of alpha-synuclein protein via an engineered guide RNA of the present disclosure result in a reduction of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of alpha-synuclein protein, relative to an amount prior to contacting the engineered guide RNA with the SNCA RNA. In some embodiments, the target SNCA RNA comprises a sequence that is at least 80% identical to SEQ ID NO: 300.

Alpha-synucleinopathies are characterized by alpha-synuclein dysfunction, overexpression and/or aggregation and are linked to neurodegenerative diseases by both genetic and neuropathological evidence. The gene encoding alpha-synuclein protein is referred to as SNCA. In Parkinson's disease (PD), SNCA genetic duplications and variants that promote alpha-synuclein aggregation (e.g., A53T) lead to early-onset and severe forms of disease. Thus, the engineered guide RNAs of the present disclosure can target SNCA for RNA editing, thereby, driving a reduction in alpha-synuclein synthesis and promoting clearance of aggregation. In some embodiments, the present disclosure provides compositions of engineered guide RNAs that target SNCA and facilitated ADAR-mediated RNA editing of SNCA to reduce pathogenic levels of alpha-synuclein by targeting key adenosines for deamination that are present in the translational initiation sites (TISs). In some embodiments, the engineered guide RNAs of the present disclosure target a coding sequence in SNCA. For example, the coding sequence can be a translation initiation site (TIS) (AUG) of SNCA and the engineered guide RNA can facilitate ADAR-mediated RNA editing of AUG to GUG. Thus, engineered guide RNAs of the present disclosure targeting these sites in SNCA are capable of facilitating edits that result in reduction in expression of the alpha-synuclein protein. In some embodiments, the TIS targeted by the engineered guide RNAs of the present disclosure is in Codon 1 of SNCA. In some embodiments, the engineered guide RNAs of the present disclosure target any key adenosine in the SNCA native TIS. For example, in some embodiments, the engineered guide RNAs target the AUG at position 265 in Exon 2 of SNCA to facilitate ADAR-mediated editing to GUG, thereby reducing alpha-synuclein expression. Assays to determine successful RNA editing can include NGS, Sanger sequencing, qPCR, ddPCR, fluorometric Western blots, and an alpha-synuclein specific sandwich ELISA. In some embodiments, any of the engineered guide RNAs disclosed herein are packaged in an AAV vector and are virally delivered. In some embodiments, administering a composition provided herein is sufficient to reduce expression of alpha-synuclein protein in a subject relative to an amount of alpha-synuclein protein prior to the administering, as determined by an in-vitro assay or an in-vivo assay. In some embodiments, the in-vitro assay comprises an immunosorbent assay or a sequencing assay. In some embodiments, the in-vivo assay comprises obtaining a biological sample from the subject and performing an in-vitro assay, for example, an enzyme-linked immunosorbent assay (ELISA).

As disclosed herein, editing of a target sequence of a target SNCA RNA (e.g., SEQ ID NO: 300) by an engineered guide RNA via ADAR can be used to reduce expression of alpha-synuclein protein. A reduction in alpha-synuclein can be utilized to treat a disease of condition associated with alpha-synuclein. In some embodiments, the disease or condition is a synucleinopathy. Editing of a target SNCA RNA as described herein, with concomitant reduction in alpha-synuclein levels, can be utilized to reduce or prevent aggregation of alpha-synuclein protein. Thus, one or more symptoms associated with aggregation of alpha-synuclein (e.g. synucleinopathies) can be treated by administration of an engineered guide RNA described herein.

As disclosed herein, administration of an engineered guide RNA described herein that targets SNCA RNA to a subject can be used to treat a disease or condition associated with alpha-synuclein, including treatment of one or more symptoms associated with the disease or condition. In some embodiments, the disease or the condition is associated with alpha-synuclein aggregation in the brain of a subject. In some embodiments, the disease or the condition is at least one selected from the group consisting of: a neurodegenerative disease, a Parkinson's Disease, tremors, muscle stiffness, muscle rigidity, bradykinesia, Dementia with Lewy Bodies (DLB), Multiple System Atrophy (MSA), Pure Autonomic Failure (PAF) and REM sleep Behavior Disorder (RBD). In some embodiments, the disease or condition can be Parkinson's disease. In some embodiments, one or more symptoms of Parkinson's disease can be treated by administration of an engineered guide RNA targeting SNCA RNA as described herein. For example, administration of an engineered guide RNA can be sufficient to reduce resting tremors, muscle stiffness, difficulty standing, difficulty walking, difficulty with bodily movements, involuntary movements, muscle rigidity, problems with coordination, rhythmic muscle contractions, slow bodily movement, bradykinesia, slow shuffling gait, or any combination thereof. In some embodiments, treatment of Parkinson's disease comprises improvement in cognitive function. For example, a subject administered an engineered guide RNA targeting SNCA of the present disclosure can display an increase in a cognitive aptitude or motor skill test, relative to performance prior to the administering. In some embodiments, a subject can show improvement in a Unified Parkinson's Disease Rating Scale (UPDRS) test, such as an MDS-UPDRS test. In some embodiments, a subject can be evaluated via an imaging technique such as an MRI or CAT scan in order to monitor the progression of the disease or condition. For example, MRI imaging can be used to visualize neurons of a subject over a treatment duration to monitor the progression of treatment. In some embodiments, neuronal cells in the substantia nigra can be monitored for degradation throughout the treatment duration.

As disclosed herein, administration of an engineered guide RNA of the present disclosure can be used to reduce alpha-synuclein protein levels through knockdown in order to treat a disease or condition associated with alpha-synuclein. While a reduction is obtained through administration, residual alpha-synuclein can still be present after the administering. In some cases, the presence of reduced alpha-synuclein protein levels treats the disease or condition, without reducing the level of alpha-synuclein protein levels to zero. Such levels can be determined in an in-vitro assay using a sample obtained from a subject. In some instances, the levels can be determined in-vivo using, for example, an imaging technique such as MRI as described above. Treatment can result in improvement in certain biomarkers in subject. For example, treatment can result in reduction of SNCA in CSF, reduction of SNCA in blood, reduced levels of Neurofilament A in CSF, or any combination thereof.

In some embodiments, the engineered guide RNAs of the present disclosure facilitate ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5% editing of a target adenosine. In some embodiments, the engineered guide RNAs of the present disclosure can facilitate at least 10% editing of a target adenosine. 15% editing of a target adenosine. In some embodiments, the engineered guide RNAs of the present disclosure can facilitate at least 20% editing of a target adenosine. In some embodiments, the engineered guide RNAs of the present disclosure can facilitate at least 25% editing of a target adenosine. In some embodiments, the engineered guide RNAs of the present disclosure can facilitate at least 30% editing of a target adenosine. In some embodiments, the engineered guide RNAs of the present disclosure can facilitate at least 35% editing of a target adenosine. In some embodiments, the engineered guide RNAs of the present disclosure can facilitate at least 40% editing of a target adenosine. In some embodiments, the engineered guide RNAs of the present disclosure can facilitate at least 45% editing of a target adenosine. In some embodiments, the engineered guide RNAs of the present disclosure can facilitate at least 50% editing of a target adenosine. In some embodiments, the engineered guide RNAs of the present disclosure can facilitate at least 55% editing of a target adenosine. In some embodiments, the engineered guide RNAs of the present disclosure can facilitate at least 60% editing of a target adenosine. In some embodiments, the engineered guide RNAs of the present disclosure can facilitate at least 65% editing of a target adenosine. In some embodiments, the engineered guide RNAs of the present disclosure can facilitate at least 70% editing of a target adenosine. In some embodiments, the engineered guide RNAs of the present disclosure can facilitate at least 75% editing of a target adenosine. In some embodiments, the engineered guide RNAs of the present disclosure can facilitate at least 80% editing of a target adenosine. In some embodiments, the engineered guide RNAs of the present disclosure can facilitate at least 85% editing of a target adenosine. In some embodiments, the engineered guide RNAs of the present disclosure can facilitate at least 90% editing of a target adenosine. In some embodiments, the engineered guide RNAs of the present disclosure can facilitate at least 95% editing of a target adenosine. In some embodiments, the engineered guide RNAs of the present disclosure can facilitate 100% editing of a target adenosine. In some embodiments, the engineered guide RNAs of the present disclosure can facilitate from 5 to 20% editing of a target adenosine. In some embodiments, the engineered guide RNAs of the present disclosure can facilitate from 20 to 40% editing of a target adenosine. In some embodiments, the engineered guide RNAs of the present disclosure can facilitate from 40 to 60% editing of a target adenosine. In some embodiments, the engineered guide RNAs of the present disclosure can facilitate from 60 to 80% editing of a target adenosine. In some embodiments, the engineered guide RNAs of the present disclosure can facilitate from 80 to 100% editing of a target adenosine. In some embodiments, the engineered guide RNAs of the present disclosure can facilitate from 60 to 80% editing of a target adenosine. In some embodiments, the engineered guide RNAs of the present disclosure can facilitate from 70 to 90% editing of a target adenosine.

In some embodiments, the engineered guide RNAs of the present disclosure can facilitate at least 70% or more editing of a target adenosine. In some embodiments, the engineered guide RNAs of the present disclosure can facilitate at least 80% or more editing of a target adenosine. In some embodiments, the engineered guide RNAs of the present disclosure can facilitate up to 90% or more editing of a target adenosine. Optionally, additionally, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 30% editing of an off-target adenosine. In some embodiments, the engineered guide RNAs of the present disclosure can facilitate these levels of on-target RNA editing while maintaining less than 25% editing of an off-target adenosine. In some embodiments, the engineered guide RNAs of the present disclosure can facilitate these levels of on-target RNA editing while maintaining less than 20% editing of an off-target adenosine. In some embodiments, the engineered guide RNAs of the present disclosure can facilitate these levels of on-target RNA editing while maintaining less than 15% editing of an off-target adenosine. In some embodiments, the engineered guide RNAs of the present disclosure can facilitate these levels of on-target RNA editing while maintaining less than 10% editing of an off-target adenosine. In some embodiments, the engineered guide RNAs of the present disclosure can facilitate these levels of on-target RNA editing while maintaining less than 9% editing of an off-target adenosine. In some embodiments, the engineered guide RNAs of the present disclosure can facilitate these levels of on-target RNA editing while maintaining less than 8% editing of an off-target adenosine. In some embodiments, the engineered guide RNAs of the present disclosure can facilitate these levels of on-target RNA editing while maintaining less than 7% editing of an off-target adenosine. In some embodiments, the engineered guide RNAs of the present disclosure can facilitate these levels of on-target RNA editing while maintaining less than 6% editing of an off-target adenosine. In some embodiments, the engineered guide RNAs of the present disclosure can facilitate these levels of on-target RNA editing while maintaining less than 5% editing of an off-target adenosine. In some embodiments, the engineered guide RNAs of the present disclosure can facilitate these levels of on-target RNA editing while maintaining less than 4% editing of an off-target adenosine. In some embodiments, the engineered guide RNAs of the present disclosure can facilitate these levels of on-target RNA editing while maintaining less than 3% editing of an off-target adenosine. In some embodiments, the engineered guide RNAs of the present disclosure can facilitate these levels of on-target RNA editing while maintaining less than 2% editing of an off-target adenosine. In some embodiments, the engineered guide RNAs of the present disclosure can facilitate these levels of on-target RNA editing while maintaining less than 1% editing of an off-target adenosine. In some embodiments, the engineered guide RNAs of the present disclosure can facilitate these levels of on-target RNA editing while maintaining 0% editing of an off-target adenosine. In some embodiments, the engineered guide RNAs of the present disclosure can facilitate at least 70% editing of a target adenosine while maintaining less than 30% editing of an off-target adenosine. In some embodiments, the engineered guide RNAs of the present disclosure can facilitate at least 70% editing of a target adenosine while maintaining less than 29% editing of an off-target adenosine. In some embodiments, the engineered guide RNAs of the present disclosure can facilitate at least 70% editing of a target adenosine while maintaining less than 28% editing of an off-target adenosine. In some embodiments, the engineered guide RNAs of the present disclosure can facilitate at least 70% editing of a target adenosine while maintaining less than 27% editing of an off-target adenosine. In some embodiments, the engineered guide RNAs of the present disclosure can facilitate at least 70% editing of a target adenosine while maintaining less than 26% editing of an off-target adenosine. In some embodiments, the engineered guide RNAs of the present disclosure can facilitate at least 70% editing of a target adenosine while maintaining less than 25% editing of an off-target adenosine. In some embodiments, the engineered guide RNAs of the present disclosure can facilitate at least 70% editing of a target adenosine while maintaining less than 24% editing of an off-target adenosine. In some embodiments, the engineered guide RNAs of the present disclosure can facilitate at least 70% editing of a target adenosine while maintaining less than 23% editing of an off-target adenosine. In some embodiments, the engineered guide RNAs of the present disclosure can facilitate at least 70% editing of a target adenosine while maintaining less than 22% editing of an off-target adenosine. In some embodiments, the engineered guide RNAs of the present disclosure can facilitate at least 70% editing of a target adenosine while maintaining less than 21% editing of an off-target adenosine. In some embodiments, the engineered guide RNAs of the present disclosure can facilitate at least 70% editing of a target adenosine while maintaining less than 20% editing of an off-target adenosine. In some embodiments, the engineered guide RNAs of the present disclosure can facilitate at least 70% editing of a target adenosine while maintaining less than 19% editing of an off-target adenosine. In some embodiments, the engineered guide RNAs of the present disclosure can facilitate at least 70% editing of a target adenosine while maintaining less than 18% editing of an off-target adenosine. In some embodiments, the engineered guide RNAs of the present disclosure can facilitate at least 70% editing of a target adenosine while maintaining less than 17% editing of an off-target adenosine. In some embodiments, the engineered guide RNAs of the present disclosure can facilitate at least 70% editing of a target adenosine while maintaining less than 16% editing of an off-target adenosine. In some embodiments, the engineered guide RNAs of the present disclosure can facilitate at least 70% editing of a target adenosine while maintaining less than 15% editing of an off-target adenosine. In some embodiments, the engineered guide RNAs of the present disclosure can facilitate at least 70% editing of a target adenosine while maintaining less than 14% editing of an off-target adenosine. In some embodiments, the engineered guide RNAs of the present disclosure can facilitate at least 70% editing of a target adenosine while maintaining less than 13% editing of an off-target adenosine. In some embodiments, the engineered guide RNAs of the present disclosure can facilitate at least 70% editing of a target adenosine while maintaining less than 12% editing of an off-target adenosine. In some embodiments, the engineered guide RNAs of the present disclosure can facilitate at least 70% editing of a target adenosine while maintaining less than 11% editing of an off-target adenosine. In some embodiments, the engineered guide RNAs of the present disclosure can facilitate at least 70% editing of a target adenosine while maintaining less than 10% editing of an off-target adenosine. In some embodiments, the engineered guide RNAs of the present disclosure can facilitate at least 70% editing of a target adenosine while maintaining less than 9% editing of an off-target adenosine. In some embodiments, the engineered guide RNAs of the present disclosure can facilitate at least 70% editing of a target adenosine while maintaining less than 8% editing of an off-target adenosine. In some embodiments, the engineered guide RNAs of the present disclosure can facilitate at least 70% editing of a target adenosine while maintaining less than 7% editing of an off-target adenosine. In some embodiments, the engineered guide RNAs of the present disclosure can facilitate at least 70% editing of a target adenosine while maintaining less than 6% editing of an off-target adenosine. In some embodiments, the engineered guide RNAs of the present disclosure can facilitate at least 70% editing of a target adenosine while maintaining less than 5% editing of an off-target adenosine. In some embodiments, the engineered guide RNAs of the present disclosure can facilitate at least 70% editing of a target adenosine while maintaining less than 4% editing of an off-target adenosine. In some embodiments, the engineered guide RNAs of the present disclosure can facilitate at least 70% editing of a target adenosine while maintaining less than 3% editing of an off-target adenosine. In some embodiments, the engineered guide RNAs of the present disclosure can facilitate at least 70% editing of a target adenosine while maintaining less than 2% editing of an off-target adenosine. In some embodiments, the engineered guide RNAs of the present disclosure can facilitate at least 70% editing of a target adenosine while maintaining less than 1% editing of an off-target adenosine. In some embodiments, the engineered guide RNAs of the present disclosure can facilitate at least 70% editing of a target adenosine while maintaining 0% editing of an off-target adenosine.

In some embodiments, the engineered guide RNAs of the present disclosure facilitate ADAR-mediated RNA editing of SNCA, which results in knockdown of protein levels. The knockdown in protein levels is quantitated as a reduction in expression of the alpha-synuclein protein. The engineered guide RNAs of the present disclosure can facilitate from 1% to 100% alpha-synuclein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90% alpha-synuclein knockdown. In some embodiments, the engineered guide RNAs of the present disclosure facilitate from 30% to 60% alpha-synuclein knockdown. Alpha-synuclein knockdown can be measured by an assay comparing a sample or subject treated with the engineered guide RNA to a control sample or subject not treated with the engineered guide RNA.

An engineered guide RNA of the present disclosure can be used in a method of treating a disorder in a subject in need thereof. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., a synucleinopathy such as Parkinson's disease). In some embodiments, an engineered guide RNA of the present disclosure can be used to treat a condition associated with one or more mutations.

### Pharmaceutical Compositions

The compositions described herein (e.g., compositions comprising an engineered guide RNA or an engineered polynucleotide) can be formulated with a pharmaceutically acceptable carrier for administration to a subject (e.g., a human or a non-human animal). A pharmaceutically acceptable carrier can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

In some examples, the pharmaceutical composition can be formulated in unit dose forms or multiple-dose forms. In some examples, the unit dose forms can be physically discrete units suitable for administration to human or non-human subjects (e.g., animals). In some examples, the unit dose forms can be packaged individually. In some examples, each unit dose contains a predetermined quantity of an active ingredient(s) that can be sufficient to produce the desired therapeutic effect in association with pharmaceutical carriers, diluents, excipients, or any combination thereof. In some examples, the unit dose forms comprise ampules, syringes, or individually packaged tablets and capsules, or any combination thereof. In some instances, a unit dose form can be comprised in a disposable syringe. In some instances, unit-dosage forms can be administered in fractions or multiples thereof. In some examples, a multiple-dose form comprises a plurality of identical unit dose forms packaged in a single container, which can be administered in segregated a unit dose form. In some examples, multiple dose forms comprise vials, bottles of tablets or capsules, or bottles of pints or gallons. In some instances, a multiple-dose forms comprise the same pharmaceutically active agents. In some instances, a multiple-dose forms comprise different pharmaceutically active agents.

In some examples, the pharmaceutical composition comprises a pharmaceutically acceptable excipient. In some examples, the excipient comprises a buffering agent, a cryopreservative, a preservative, a stabilizer, a binder, a compaction agent, a lubricant, a chelator, a dispersion enhancer, a disintegration agent, a flavoring agent, a sweetener, or a coloring agent, or any combination thereof.

In some examples, an excipient comprises a buffering agent. In some examples, the buffering agent comprises sodium citrate, magnesium carbonate, magnesium bicarbonate, calcium carbonate, calcium bicarbonate, or any combination thereof. In some examples, the buffering agent comprises sodium bicarbonate, potassium bicarbonate, magnesium hydroxide, magnesium lactate, magnesium gluconate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, or calcium hydroxide and other calcium salts, or any combination thereof.

In some examples, an excipient comprises a cryopreservative. In some examples, the cryopreservative comprises DMSO, glycerol, polyvinylpyrrolidone (PVP), or any combination thereof. In some examples, a cryopreservative comprises a sucrose, a trehalose, a starch, a salt of any of these, a derivative of any of these, or any combination thereof. In some examples, an excipient comprises a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. In some examples, an excipient comprises a surfactant, a sugar, an amino acid, an antioxidant, a salt, a non-ionic surfactant, a solubilizer, a triglyceride, an alcohol, or any combination thereof. In some examples, an excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerin, xanthan rubber, soy isoflavones, polysorbate 80, ethyl alcohol, water, teprenone, or any combination thereof. In some examples, the excipient can be an excipient described in the Handbook of Pharmaceutical Excipients, American Pharmaceutical Association (1986).

In some examples, the excipient comprises a preservative. In some examples, the preservative comprises an antioxidant, such as alpha-tocopherol and ascorbate, an antimicrobial, such as parabens, chlorobutanol, and phenol, or any combination thereof. In some examples, the antioxidant comprises EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol or N-acetyl cysteine, or any combination thereof. In some examples, the preservative comprises validamycin A, TL-3, sodium ortho vanadate, sodium fluoride, N-α-tosyl-Phe-chloromethylketone, N-α-tosyl-Lys-chloromethylketone, aprotinin, phenylmethylsulfonyl fluoride, diisopropylfluorophosphate, kinase inhibitor, phosphatase inhibitor, caspase inhibitor, granzyme inhibitor, cell adhesion inhibitor, cell division inhibitor, cell cycle inhibitor, lipid signaling inhibitor, protease inhibitor, reducing agent, alkylating agent, antimicrobial agent, oxidase inhibitor, or other inhibitors, or any combination thereof.

In some examples, the excipient comprises a binder. In some examples, the binder comprises starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, or any combination thereof.

In some examples, the binder can be a starch, for example a potato starch, corn starch, or wheat starch; a sugar such as sucrose, glucose, dextrose, lactose, or maltodextrin; a natural and/or synthetic gum; a gelatin; a cellulose derivative such as microcrystalline cellulose, hydroxypropyl cellulose, hydroxyethyl cellulose, hydroxypropyl methyl cellulose, carboxymethyl cellulose, methyl cellulose, or ethyl cellulose; polyvinylpyrrolidone (povidone); polyethylene glycol (PEG); a wax; calcium carbonate; calcium phosphate; an alcohol such as sorbitol, xylitol, mannitol, or water, or any combination thereof.

In some examples, the excipient comprises a lubricant. In some examples, the lubricant comprises magnesium stearate, calcium stearate, zinc stearate, hydrogenated vegetable oils, hydrogenated vegetable oil (STEROTEX®), hydrogenated cottonseed oil, polyoxyethylene monostearate, talc, polyethyleneglycol, sodium benzoate, sodium lauryl sulfate, magnesium lauryl sulfate, or light mineral oil, or any combination thereof. In some examples, the lubricant comprises metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate or talc or a combination thereof.

In some examples, the excipient comprises a dispersion enhancer. In some examples, the dispersion enhancer comprises starch, alginic acid, polyvinylpyrrolidones, guar gum, kaolin, bentonite, purified wood cellulose, sodium starch glycolate, isomorphous silicate, or microcrystalline cellulose, or any combination thereof as high HLB emulsifier surfactants.

In some examples, the excipient comprises a disintegrant. In some examples, a disintegrant comprises a non-effervescent disintegrant. In some examples, a non-effervescent disintegrants comprises starches such as corn starch, potato starch, pregelatinized and modified starches thereof, sweeteners, clays, such as bentonite, micro-crystalline cellulose, alginates, sodium starch glycolate, or gums such as agar, guar, locust bean, karaya, pectin, and tragacanth, or any combination thereof. In some examples, a disintegrant comprises an effervescent disintegrant. In some examples, a suitable effervescent disintegrant comprises bicarbonate in combination with citric acid, and sodium bicarbonate in combination with tartaric acid.

In some examples, the excipient comprises a sweetener, a flavoring agent or both. In some examples, a sweetener comprises glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia rebaudiana (Stevioside); chloro derivatives of sucrose such as sucralose; and sugar alcohols such as sorbitol, mannitol, sylitol, and the like, or any combination thereof.

In some cases, flavoring agents incorporated into a composition comprise synthetic flavor oils and flavoring aromatics; natural oils; extracts from plants, leaves, flowers, and fruits; or any combination thereof. In some embodiments, a flavoring agent comprises a cinnamon oils; oil of wintergreen; peppermint oils; clover oil; hay oil; anise oil; eucalyptus; vanilla; citrus oil such as lemon oil, orange oil, grape and grapefruit oil; and fruit essences including apple, peach, pear, strawberry, raspberry, cherry, plum, pineapple, and apricot, or any combination thereof.

In some examples, the excipient comprises a pH agent (e.g., to minimize oxidation or degradation of a component of the composition), a stabilizing agent (e.g., to prevent modification or degradation of a component of the composition), a buffering agent (e.g., to enhance temperature stability), a solubilizing agent (e.g., to increase protein solubility), or any combination thereof. In some examples, the excipient comprises a surfactant, a sugar, an amino acid, an antioxidant, a salt, a non-ionic surfactant, a solubilizer, a triglyceride, an alcohol, or any combination thereof. In some examples, the excipient comprises sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof. In some examples, the excipient comprises a cryo-preservative. In some examples, the excipient comprises DMSO, glycerol, polyvinylpyrrolidone (PVP), or any combination thereof. In some examples, the excipient comprises a sucrose, a trehalose, a starch, a salt of any of these, a derivative of any of these, or any combination thereof.

In some examples, the pharmaceutical composition comprises a diluent. In some examples, the diluent comprises water, glycerol, methanol, ethanol, or other similar biocompatible diluents, or any combination thereof. In some examples, a diluent comprises an aqueous acid such as acetic acid, citric acid, maleic acid, hydrochloric acid, phosphoric acid, nitric acid, sulfuric acid, or any combination thereof. In some examples, a diluent comprises an alkaline metal carbonates such as calcium carbonate; alkaline metal phosphates such as calcium phosphate; alkaline metal sulphates such as calcium sulphate; cellulose derivatives such as cellulose, microcrystalline cellulose, cellulose acetate; magnesium oxide, dextrin, fructose, dextrose, glyceryl palmitostearate, lactitol, choline, lactose, maltose, mannitol, simethicone, sorbitol, starch, pregelatinized starch, talc, xylitol and/or anhydrates, hydrates and/or pharmaceutically acceptable derivatives thereof or combinations thereof.

In some examples, the pharmaceutical composition comprises a carrier. In some examples, the carrier comprises a liquid or solid filler, solvent, or encapsulating material. In some examples, the carrier comprises additives proteins, peptides, amino acids, lipids, and carbohydrates (e.g., sugars, including monosaccharides, di-, tri-, tetra-oligosaccharides, and oligosaccharides; derivatized sugars such as alditols, aldolic acids, esterified sugars and the like; and polysaccharides or sugar polymers), alone or in combination.

### Delivery

An engineered guide RNA of the present disclosure (such as an engineered guide RNA that comprises a polynucleotide sequence of any one of SEQ ID NOS: 2-285, 293-299, 333-357, 375-376, 380-381, 390-440, or 777-784) or an engineered polynucleotide of the present disclosure (e.g., an engineered polynucleotide encoding an engineered guide RNA of the present disclosure, such as an engineered polynucleotide that comprises a polynucleotide sequence of any one of SEQ ID NOS: 286-292, 325-332, 358-379, 441-776, or 785-792) can be delivered via a delivery vehicle. In some embodiments, the delivery vehicle is a vector. A vector can facilitate delivery of the engineered guide RNA into a cell to genetically modify the cell. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the viral vector can be of a specific serotype. In some embodiments, the viral vector can be an AAV1 serotype, AAV2 serotype, AAV3 serotype, AAV4 serotype, AAV5 serotype, AAV6 serotype, AAV7 serotype, AAV8 serotype, AAV9 serotype, AAV10 serotype, AAV11 serotype, AAV 12 serotype, AAV13 serotype, AAV14 serotype, AAV15 serotype, AAV16 serotype, AAV.rh8 serotype, AAV.rh10 serotype, AAV.rh20 serotype, AAV.rh39 serotype, AAV.Rh74 serotype, AAV.RHM4-1 serotype, AAV.hu37 serotype, AAV.Anc80 serotype, AAV.Anc80L65 serotype, AAV.7m8 serotype, AAV.PHP.B serotype, AAV2.5 serotype, AAV2tYF serotype, AAV3B serotype, AAV.LK03 serotype, AAV.HSC1 serotype, AAV.HSC2 serotype, AAV.HSC3 serotype, AAV.HSC4 serotype, AAV.HSC5 serotype, AAV.HSC6 serotype, AAV.HSC7 serotype, AAV.HSC8 serotype, AAV.HSC9 serotype, AAV.HSC10 serotype, AAV.HSC11 serotype, AAV.HSC12 serotype, AAV.HSC13 serotype, AAV.HSC14 serotype, AAV.HSC15 serotype, AAV.HSC16 serotype, and AAVhu68 serotype, a derivative of any of these serotypes, or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding an engineered guide RNA) in an AAV vector. In some examples, methods of producing the delivery vectors described herein comprise, (a) introducing into a cell: (i) a polynucleotide comprising a promoter and an engineered guide RNA disclosed herein; and (ii) a viral genome comprising a Replication (Rep) gene and Capsid (Cap) gene that encodes a wild-type AAV capsid protein or modified version thereof; (b) expressing in the cell the wild-type AAV capsid protein or modified version thereof; (c) assembling an AAV particle; and (d) packaging the engineered guide RNA disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/6, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system comprises can be a liposome or polymeric nanoparticle. In some embodiments, the engineered polynucleotide or a non-viral vector comprising the engineered polynucleotide is delivered to a cell by hydrodynamic injection or ultrasound.

### Administration

Administration can refer to methods that can be used to enable the delivery of a composition described herein (e.g., comprising an engineered guide RNA or an engineered polynucleotide encoding the same) to the desired site of biological action. For example, an engineered guide RNA (such as an engineered guide RNA that comprises a polynucleotide sequence of any one of SEQ ID NOS: 2-285, 293-299, 333-357, 375-376, 380-381, 390-440, or 777-784) can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracomeal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some examples, a pharmaceutical composition disclosed herein can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The appropriate dosage and treatment regimen for the methods of treatment described herein vary with respect to the particular disease being treated, the gRNA and/or ADAR (or a vector encoding the gRNA and/or ADAR) being delivered, and the specific condition of the subject. In some examples, the administration can be over a period of time until the desired effect (e.g., reduction in symptoms can be achieved). In some examples, administration can be 1, 2, 3, 4, 5, 6, or 7 times per week. In some examples, administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. In some examples, administration can be over a period of 1, 2, 3, 4, 5, 6, 7, 8, 9, or 10 weeks. In some examples, administration can be over a period of 2, 3, 4, 5, 6 or more months. In some examples, administration can be performed repeatedly over a lifetime of a subject, such as once a month or once a year for the lifetime of a subject. In some examples, administration can be performed repeatedly over a substantial portion of a subject's life, such as once a month or once a year for at least about 1 year, 5 years, 10 years, 15 years, 20 years, 25 years, 30 years, or more. In some examples, treatment can be resumed following a period of remission.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, disclosed herein can be a method, comprising administering a composition disclosed herein to a subject (e.g., a human) in need thereof. In some instances, the method can treat (including prevent) a disease in the subject.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As disclosed herein, a base paired (bp) region refers to a region of the guide-target RNA scaffold in which bases in the guide RNA (e.g., the bases in the targeting sequence of the guide RNA) are paired with opposing bases in the target polynucleotide. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold.

In some embodiments, a base paired region has from 1 to 50, 1 to 75, 1 to 100, 1 to 125, 1 to 150, 1 to 175, 1 to 200, 1 to 225, 1 to 250, 1 to 275, 1 to 300, 50 to 75, 50 to 100, 50 to 125, 50 to 150, 50 to 175, 50 to 200, 50 to 225, 50 to 250, 50 to 275, 50 to 300, 60 to 75, 60 to 100, 60 to 125, 60 to 150, 60 to 175, 60 to 200, 60 to 225, 60 to 250, 60 to 275, 60 to 300, 70 to 100, 70 to 125, 70 to 150, 70 to 175, 70 to 200, 70 to 225, 70 to 250, 70 to 275, 70 to 300, 80 to 100, 80 to 125, 80 to 150, 80 to 175, 80 to 200, 80 to 225, 80 to 250, 80 to 275, 80 to 300, 90 to 125, 90 to 150, 90 to 175, 90 to 200, 90 to 225, 90 to 250, 90 to 275, 90 to 300, 100 to 125, 100 to 150, 100 to 175, 100 to 200, 100 to 225, 100 to 250, 100 to 275, 100 to 300, 150 to 200, 150 to 225, 150 to 250, 150 to 275, or 150 to 300 base pairs. In some embodiments, a base paired region has at least 1, 2, 3, 4, 5, 6, 7, 8, 9, 10, 12, 14, 16, 18, 20, 25, 30, 35, 40, 45, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 base pairs.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode for a polypeptide during translation, whereas DNA can encode for an mRNA molecule during transcription.

As used herein, the term “engineered guide RNA” can be used interchangeable with “guide RNA” and refers to a designed polynucleotide that is at least partially complementary to a target RNA. An engineered guide RNA of the present disclosure can be used to facilitate modification of the target RNA. Modification of the target RNA includes alteration of RNA splicing, reduction or enhancement of protein translation, target RNA knockdown, target RNA degradation, and/or ADAR mediated RNA editing of the target RNA. In some cases, guide RNAs facilitate ADAR mediated RNA editing for the purpose of target mRNA knockdown, downstream protein translation reduction or inhibition, downstream protein translation enhancement, correction of mutations (including correction of any G to A mutation, such as missense or nonsense mutations), introduction of mutations (e.g., introduction of an A to I (read as a G by cellular machinery) substitution), or alter the function of any adenosine containing a regulatory motif (e.g., polyadenylation signal, miRNA binding site, etc.). In some cases, a guide RNA can affect a functional outcome (e.g., target RNA modulation, downstream protein translation) via a combination of mechanisms, for example, ADAR-mediated RNA editing and binding and/or degrading target RNA. In some cases, a guide RNA can facilitate introduction of mutations at sites targeted by enzymes in order to modify the affinity of such enzymes for targeting and cleaving such sites. The guide RNAs of this disclosure can contain one or more structural features. A structural feature can be formed from latent structure in latent (unbound) guide RNA upon hybridization of the engineered latent guide RNA to a target RNA. Latent structure refers to a structural feature that forms or substantially forms only upon hybridization of a guide RNA to a target RNA. For example, upon hybridization of the guide RNA to the target RNA, the latent structural feature is formed in the resulting double stranded RNA (also referred herein as guide-target RNA scaffold). In such cases, a structural feature can include, but is not limited to, a mismatch, a wobble base pair, a symmetric internal loop, an asymmetric internal loop, a symmetric bulge, or an asymmetric bulge. In other instances, a structural feature can be a pre-formed structure (e.g., a GluR2 recruitment hairpin, or a hairpin from U7 snRNA).

An engineered latent guide RNA refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A guide-target RNA scaffold, as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain (e.g., the targeting sequence) of the engineered guide RNAs of the present disclosure, or any combination thereof.

A recruitment hairpin, as disclosed herein, can recruit at least in part an RNA editing entity, such as ADAR. In some cases, a recruitment hairpin can be formed and present in the absence of binding to a target RNA. In some embodiments, a recruitment hairpin is a GluR2 domain or portion thereof. In some embodiments, a recruitment hairpin is an Alu domain or portion thereof. A recruitment hairpin, as defined herein, can include a naturally occurring ADAR substrate or truncations thereof. Thus, a recruitment hairpin such as GluR2 is a pre-formed structural feature that may be present in constructs comprising an engineered guide RNA, not a structural feature formed by latent structure provided in an engineered latent guide RNA.

A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in-vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

The term percent “identity,” in the context of two or more nucleic acid or polypeptide sequences, refers to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence (also called the subject sequence) to which test sequences (also called query sequences) are compared. The percent sequence identity is defined as a test sequence's percent identity to a reference sequence. For example, when stated “Sequence A having a sequence identity of 50% to Sequence B,” Sequence A is the test sequence and Sequence B is the reference sequence. When using a sequence comparison algorithm, test and reference sequences are input into a computer program, subsequence coordinates are designated, if necessary, and sequence algorithm program parameters are designated. The sequence comparison algorithm then aligns the sequences to achieve the maximum alignment, based on the designated program parameters, introducing gaps in the alignment if necessary. The percent sequence identity for the test sequence(s) relative to the reference sequence can then be determined from the alignment of the test sequence to the reference sequence. The equation for percent sequence identity from the aligned sequence is as follows: [(Number of Identical Positions)/(Total Number of Positions in the Test Sequence)]×100%.

For purposes herein, percent identity and sequence similarity calculations are performed using the BLAST algorithm for sequence alignment, which is described in Altschul et al., J. Mol. Biol. 215:403-410 (1990). Software for performing BLAST analyses is publicly available through the National Center for Biotechnology Information (www.ncbi.nlm.nih.gov/). The BLAST algorithm uses a test sequence (also called a query sequence) and a reference sequence (also called a subject sequence) to search against, or in some cases, a database of multiple reference sequences to search against. The BLAST algorithm performs sequence alignment by finding high-scoring alignment regions between the test and the reference sequences by scoring alignment of short regions of the test sequence (termed “words”) to the reference sequence. The scoring of each alignment is determined by the BLAST algorithm and takes factors into account, such as the number of aligned positions, as well as whether introduction of gaps between the test and the reference sequences would improve the alignment. The alignment scores for nucleic acids can be scored by set match/mismatch scores. For protein sequences, the alignment scores can be scored using a substitution matrix to evaluate the significance of the sequence alignment, for example, the similarity between aligned amino acids based on their evolutionary probability of substitution. For purposes herein, the substitution matrix used is the BLOSUM62 matrix. For purposes herein, the public default values of Apr. 6, 2023 are used when using the BLASTN and BLASTP algorithms. The BLASTN and BLASTP algorithms then output a “Percent Identity” output value and a “Query Coverage” output value. The overall percent sequence identity as used herein can then be calculated from the BLASTN or BLASTP output values as follows:

$$Percent Sequence Identity = ( “ Percent Identity ” output value ) \times ( “ Query Coverage ” output value ) .$$

The following non-limiting examples illustrate the calculation of percent identity between two nucleic acids sequences. The percent identity is calculated as follows: [(number of identical nucleotide positions)/(total number of nucleotides in the test sequence)]×100%. Percent identity is calculated to compare test sequence 1: AAAAAGGGGG (length=10 nucleotides) to reference sequence 2: AAAAAAAAAA (length=10 nucleotides). The percent identity between test sequence 1 and reference sequence 2 would be [(5)/(10)]×100%=50%. Test sequence 1 has 50% sequence identity to reference sequence 2. In another example, percent identity is calculated to compare test sequence 3: CCCCCGGGGGGGGGGCCCCC (length=20 nucleotides) to reference sequence 4: GGGGGGGGGG (length=10 nucleotides). The percent identity between test sequence 3 and reference sequence 4 would be [(10)/(20)]×100%=50%. Test sequence 3 has 50% sequence identity to reference sequence 4. In another example, percent identity is calculated to compare test sequence 5: GGGGGGGGGG (length=10 nucleotides) to reference sequence 6: CCCCCGGGGGGGGGGCCCCC (length=20 nucleotides). The percent identity between test sequence 5 and reference sequence 6 would be [(10)/(10)]×100%=100%. Test sequence 5 has 100% sequence identity to reference sequence 6.

The following non-limiting examples illustrate the calculation of percent identity between two protein sequences. The percent identity is calculated as follows: [(number of identical amino acid positions)/(total number of amino acids in the test sequence)]×100%. Percent identity is calculated to compare test sequence 7: FFFFFYYYYY (length=10 amino acids) to reference sequence 8: YYYYYYYYYY (length=10 amino acids). The percent identity between test sequence 7 and reference sequence 8 would be [(5)/(10)]×100%=50%. Test sequence 7 has 50% sequence identity to reference sequence 8. In another example, percent identity is calculated to compare test sequence 9: LLLLLFFFFFYYYYYLLLLL (length=20 amino acids) to reference sequence 10: FFFFFYYYYY (length=10 amino acids). The percent identity between test sequence 9 and reference sequence 10 would be [(10)/(20)]×100%=50%. Test sequence 9 has 50% sequence identity to reference sequence 10. In another example, percent identity is calculated to compare test sequence 11: FFFFFYYYYY (length=10 amino acids) to reference sequence 12: LLLLLFFFFFYYYYYLLLLL (length=20 amino acids). The percent identity between test sequence 11 and reference sequence 12 would be [(10)/(10)]×100%=100%. Test sequence 11 has 100% sequence identity to reference sequence 12.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

Latent structure refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed, and the latent structure provided in the guide RNA is, thus, unmasked. The formation and structure of a latent structural feature upon binding to the target RNA depends on the guide RNA sequence. For example, formation and structure of the latent structural feature may depend on a pattern of complementary and mismatched residues in the guide RNA sequence relative to the target RNA. The guide RNA sequence may be engineered to have a latent structural feature that forms upon binding to the target RNA.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” refers to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids. A sequence of a polynucleotide may be provided interchangeably as an RNA sequence (containing U) or a DNA sequence (containing T). A sequence provided as an RNA sequence is intended to also cover the corresponding DNA sequence and the reverse complement RNA sequence or DNA sequence. A sequence provided as a DNA sequence is intended to also cover the corresponding RNA sequence and the reverse complement RNA sequence or DNA sequence.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together −optionally to preserve the conformation of the fused protein domains, prevent unfavorable interactions between the fused protein domains which can compromise their respective functions, or both.

An engineered polynucleotide as described herein can comprise one or more polynucleotide sequence(s) that encode one or more engineered guide RNA(s). For example, an engineered polynucleotide can comprise 1, 2, 3, 4, or more than 4 polynucleotide sequence(s) that encode 1, 2, 3, 4, or more than 4 engineered guide RNAs.

In some instances, the engineered polynucleotide can comprise one or more polynucleotide sequence(s) encoding one or more engineered guide RNA(s) that independently hybridize to (target): (1) different target sequences of the same target RNA, or (2) different target sequences of different target RNAs. For example, a first engineered guide RNA encoded by a first polynucleotide sequence can hybridize to a target sequence of a first target RNA while a second engineered guide RNA encoded by a second polynucleotide sequence can hybridize to a target sequence of a second target RNA, in some instances resulting in ADAR-mediated editing of an adenosine in the target sequence of the first target RNA and an adenosine in the target sequence of the second target RNA.

In some instances, the engineered polynucleotide can comprise one or more polynucleotide sequence(s) encoding one or more engineered guide RNA(s) that independently hybridize to (target) the same target sequence of a target RNA. For example, the one or more engineered guide RNA(s) encoded by the one or more polynucleotide sequence(s) can each independently hybridize to a target sequence of a target RNA and/or facilitate editing of the same adenosine in the target sequence of the target RNA via ADAR. In some cases, the one or more engineered guide RNA(s) that hybridize to (target) the same target sequence of a target RNA have identical sequences (i.e., the one or more engineered guide RNAs are copies of each other).

Alternatively, two or more engineered guide RNA(s) that hybridize to (target) the same target sequence of a target RNA can comprise different sequences. For example, a first engineered guide RNA encoded by an engineered polynucleotide can have a sequence identity of less than, greater than, or equal to: 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% to a second engineered guide RNA encoded by the engineered polynucleotide, where the second engineered guide RNA hybridizes to (targets) the same target sequence of a target RNA as the first engineered guide RNA. In some instances, a first engineered guide RNA encoded by an engineered polynucleotide can have at least about 70% to about 99% sequence identity, at least about 60% to about 99% sequence identity, at least about 80% to about 99% sequence identity, at least about 60% to about 70% sequence identity, at least about 70% to about 80% sequence identity, at least about 75% to about 85% sequence identity, at least about 85% to about 99% sequence identity, at least about 85% to about 90% sequence identity, at least about 88% to about 93% sequence identity, at least about 90% to about 95% sequence identity, at least about 92% to about 99% sequence identity, or at least about 95% to about 99% sequence identity to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 60% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 61% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 62% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 63% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 64% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 65% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 66% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 67% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 68% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 69% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 70% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 71% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 72% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 73% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 74% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 75% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 76% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 77% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 78% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 79% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 80% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 81% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 82% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 83% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 84% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 85% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 86% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 87%, to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 88% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 89% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 90% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 91% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 92% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 93% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 94% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 95% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 96% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 97% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 98% to a second engineered guide RNA encoded by the engineered polynucleotide, where the second engineered guide RNA hybridizes to (targets) the same target sequence of a target RNA as the first engineered guide RNA. In some cases, a first engineered guide RNA encoded by an engineered polynucleotide can have a sequence identity of less than, greater than, or equal to about 99% to a second engineered guide RNA encoded by the engineered polynucleotide, where the second engineered guide RNA hybridizes to (targets) the same target sequence of a target RNA as the first engineered guide RNA. In some embodiments, polynucleotides encoding a first engineered guide RNA, a second engineered guide RNA, or both can be delivered via an AAV. In some instances, the AAV can be formulated in a composition, such as any of the pharmaceutical compositions disclosed herein.

As disclosed herein, a structured motif comprises two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in-vivo or cultured in-vitro. The subject can be a mammal. The mammal can be a human. The subject can be diagnosed or suspected of being at high risk for a disease. In some cases, the subject is not necessarily diagnosed or suspected of being at high risk for the disease

As used herein, the term “targeting sequence” can be used interchangeable with “targeting domain” or “targeting region” and refers to a polynucleotide sequence within an engineered guide RNA sequence that is at least partially complementary to a target polynucleotide. The target polynucleotide (e.g., a target RNA or a target DNA) may be a region of a polynucleotide of interest, such as a gene or a messenger RNA. As used herein, a “complementary” sequence refers to a sequence that is a reverse complement relative to a second sequence. A targeting sequence of an engineered guide RNA allows the engineered guide RNA to hybridize to a target polynucleotide (e.g., a target RNA) through base pairing, such as Watson Crick base pairing. A targeting sequence can be located at either the N-terminus or C-terminus of the engineered guide RNA, or both, or the targeting sequence can be within the engineered guide RNA. The targeting sequence can be of any length sufficient to hybridize with the target polynucleotide. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides in length. In an embodiment, an engineered polynucleotide comprises a targeting sequence that is about 25 to 200, 50 to 150, 75 to 100, 80 to 110, 90 to 120, 95 to 115, 60 to 200, 60 to 180, 60 to 160, 60 to 140, 70 to 200, 70 to 180, 70 to 160, 70 to 140, 80 to 200, 80 to 190, 80 to 170, 80 to 160, 80 to 150, 80 to 140, 80 to 130, 80 to 120, 90 to 200, 90 to 190, 90 to 180, 90 to 170, 90 to 160, 90 to 150, 90 to 140, 90 to 130, 90 to 120, 100 to 200, 100 to 190, 100 to 180, 100 to 170, 100 to 160, 100 to 150, 100 to 140, 100 to 130, 100 to 120, 110 to 200, 110 to 190, 110 to 180, 110 to 170, 110 to 160, 110 to 150, 110 to 140, 110 to 120, 120 to 200, 120 to 190, 120 to 180, 120 to 170, 120 to 160, 120 to 150, 120 to 140, 130 to 200, 130 to 190, 130 to 180, 130 to 170, 130 to 160, 130 to 150, 140 to 200, 140 to 190, 140 to 180, 140 to 170, 140 to 160, 150 to 200, 150 to 190, 150 to 180, 150 to 170, 160 to 200, 160 to 190 or 160 to 180 nucleotides in length.

A targeting sequence comprises at least partial sequence complementarity to a target polynucleotide. The targeting sequence may have a degree of sequence complementarity to the target polynucleotide sufficient to hybridize with the target polynucleotide. In some cases, the targeting sequence comprises 95%, 96%, 97%, 98%, 99%, or 100% sequence complementarity to the target polynucleotide. In some cases, the targeting sequence comprises less than 100% complementarity to the target polynucleotide sequence. For example, the targeting sequence may have a single base mismatch relative to the target polynucleotide when bound to the target polynucleotide. In other cases, the targeting sequence comprises at least about 1, 2, 3, 4, 5, 6, 7, 8, 9, 10, 11, 12, 13, 14, 15, 16, 20, 30, 40 or up to about 50 base mismatches relative to the target polynucleotide when bound to the target polynucleotide. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype polynucleotide of a subject target polynucleotide.

A targeting sequence comprises nucleotide residues having complementarity to a target polynucleotide. The targeting sequence may have a number of residues with complementarity to the target polynucleotide sufficient to hybridize with the target polynucleotide. The complementary residues may be contiguous or non-contiguous. In some cases, the targeting sequence comprises at least 50 nucleotides having complementarity to the target polynucleotide. In some cases, the targeting sequence comprises from 50 to 150 nucleotides having complementarity to the target polynucleotide. In some cases, the targeting sequence comprises from 50 to 200 nucleotides having complementarity to the target polynucleotide. In some cases, the targeting sequence comprises from 50 to 250 nucleotides having complementarity to the target polynucleotide. In some cases, the targeting sequence comprises from 50 to 300 nucleotides having complementarity to the target polynucleotide. In some cases, the targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to the target polynucleotide. In some cases, the targeting sequence comprises more than 50 nucleotides total and has at least 50 nucleotides having complementarity to the target polynucleotide. In some cases, the targeting sequence comprises from 50 to 400 nucleotides total and has from 50 to 150 nucleotides having complementarity to the target polynucleotide. In some cases, the targeting sequence comprises from 50 to 400 nucleotides total and has from 50 to 200 nucleotides having complementarity to the target polynucleotide. In some cases, the targeting sequence comprises from 50 to 400 nucleotides total and has from 50 to 250 nucleotides having complementarity to the target polynucleotide. In some cases, the targeting sequence comprises from 50 to 400 nucleotides total and has from 50 to 300 nucleotides having complementarity to the target polynucleotide. In some cases, the at least 50 nucleotides having complementarity to the target polynucleotide are separated by one or more mismatches, one or more bulges, or one or more loops, or any combination thereof. In some cases, the from 50 to 150 nucleotides having complementarity to the target polynucleotide are separated by one or more mismatches, one or more bulges, or one or more loops, or any combination thereof. In some cases, the from 50 to 200 nucleotides having complementarity to the target polynucleotide are separated by one or more mismatches, one or more bulges, or one or more loops, or any combination thereof. In some cases, the from 50 to 250 nucleotides having complementarity to the target polynucleotide are separated by one or more mismatches, one or more bulges, or one or more loops, or any combination thereof. In some cases, the from 50 to 300 nucleotides having complementarity to the target polynucleotide are separated by one or more mismatches, one or more bulges, or one or more loops, or any combination thereof. For example, a targeting sequence comprises a total of 54 nucleotides wherein, sequentially, 25 nucleotides are complementarity to the target polynucleotide, 4 nucleotides form a bulge, and 25 nucleotides are complementarity to the target polynucleotide. As another example, a targeting sequence comprises a total of 118 nucleotides wherein, sequentially, 25 nucleotides are complementarity to the target polynucleotide, 4 nucleotides form a bulge, 25 nucleotides are complementarity to the target polynucleotide, 14 nucleotides form a loop, and 50 nucleotides are complementary to the target polynucleotide.

The term “in-vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in-vitro” assay.

The term “in-vitro” refers to an event that takes places contained in a container for holding laboratory reagent such that it can be separated from the biological source from which the material can be obtained. In-vitro assays can encompass cell-based assays in which living or dead cells can be employed. In-vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g., physiological pH, physiological temperature, physiological salt concentration, etc.).

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### EXAMPLES

The following illustrative examples are representative of embodiments of the stimulation, systems, and methods described herein and are not meant to be limiting in any way.

### Example 1

### Correlation of Hard-Wired SNCA Variants with Alpha-Synuclein Protein Levels

As disclosed herein, the engineered guide RNAs of the present disclosure target the SNCA Exon 2 TIS for treatment of neurodegenerative disease, which may result in knockdown of alpha-synuclein protein levels through ADAR-mediated editing of the Exon 2 AUG TIS to a GUG valine codon. In order to validate that ADAR-mediated editing of the adenosine of the Exon 2 TIS will have an effect on the expression of alpha-synuclein protein, a “hardwired” A>G mutation of the adenosine of the Exon 2 TIS was performed on the SNCA DNA gene. Because the hardwired mutation is performed on the SNCA DNA gene, the result is conversion of 100% of the Exon 2 TIS from AUG to GUG in the corresponding SNCA RNA transcripts.

The effect of hard-wired A to G mutations at the SNCA Exon 2 translation initiation sites (TIS) and the SNCA 3′ UTR on the expression levels of alpha-synuclein protein were determined in Sh-sy5y neuroblastoma cells. The Sh-sy5y cells were cultured according to standard protocols. Site directed mutagenesis was performed at the Exon 2 Codon 1 TIS, Exon 2 Codon 5 TIS, and the SNCA 3′ UTR. As shown in FIG. 3, mutagenesis of the SNCA Exon 2 Codon 1 TIS site from adenine (A) to guanine (G) resulted in greater than 90% knockdown of alpha-synuclein protein expression in-vitro, relative to controls.

Accordingly, this example validates targeting of the SNCA Exon 2 Codon 1 TIS in order to achieve knockdown of alpha-synuclein protein. Thus, engineered guide RNAs were prepared in the examples that follow that target the following mRNA sequence:

(SEQ ID NO: 300)
GCCAUUCGACGACAGUGUGGUGUAAAGGAAUUCAUUAGCCAUGGA
UGUAUUCAUGAAAGGACUUUCAAAGGCCAAGGAGGGAGUUGUGGC
UGCUGCUGAG.

### Example 2

### Engineered Guide RNA Compositions Targeting the SNCA Codon 1 TIS

This example describes sequences of engineered guide RNAs that target the Codon 1 TIS of Exon 2 corresponding to the canonical TIS at nucleotide position 226 of SNCA transcript variant 1 (NCBI Reference Sequence: NM_000345.4). Self-annealing RNA structures, which comprised (i) the engineered guide RNAs shown in Table 1 and (ii) the RNA sequences of the SNCA region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing by next generation sequencing (NGS). Engineered guide RNAs that displayed greater than 50% on-target editing of the SNCA TIS for ADAR1 and/or ADAR2, as quantified at a read depth of >200, are shown in Table 1. All polynucleotide sequences encoding for the engineered guide RNA of Table 1, are also encompassed herein, which are represented by each of the sequences shown in Table 1, with a T substituted for each U. For each sequence, the structural features formed in the double stranded RNA substrate upon hybridization of the guide RNA to the target SNCA RNA, are shown in the second column of Table 1. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, in SEQ ID NO: 2, “−18_6-6_internal loop-symmetric_UGGUGU-UGGAGU” is read as a structural feature formed in a guide-target RNA scaffold (target SNCA RNA sequence hybridized to an engineered guide RNA of SEQ ID NO: 2), where
a. the structural feature starts 18 nucleotides upstream (5′) (the −18 position) from the target A (0 position) of the target RNA sequence
b. six contiguous bases from the target RNA sequence and six contiguous bases from the engineered guide RNA form the structural feature
c. the structural feature is a symmetric loop
d. a sequence of UGGUGU from the target RNA side and a sequence of UGGAGU from the engineered guide RNA side participate in forming the symmetric loop.

Table 1 further includes the amount of on target editing achieved via ADAR1 or ADAR2 separately, as well as ADAR1 and ADAR2. The specificity of each guide was also calculated for each engineered guide via ADAR1 and ADAR2. Specificity as provided in Table 1 was calculated using the formula: Specificity=(fraction on-target editing+1)/(sum(non-synonymous off-target editing)). These data highlight the diverse sequence space represented by the SNCA TIS-targeting engineered guide RNAs of the present disclosure, which have a range of different structural features that drive sequence diversity and which exhibit high on-target editing efficiency.

TABLE 1
Engineered Guide RNAs Targeting the SNCA Codon 1 TIS

| RNA | | | DNA | | | | | |
|---|---|---|---|---|---|---|---|---|
| SEQ | | | SEQ | Engineered | | | | |
| ID | Structural Features | Engineered Guide | ID | Guide DNA | ADAR1 | ADAR2 | ADAR1 | ADAR2 |
| NO | (target/guide) | RNA Sequence | NO | Sequence | on-target | on-target | specificity | specificity |
| 2 | −18_6-6_internal_loop- | CUCAGCAGCAGCCA | 441 | CTCAGCAG | 0.571836 | 0.571836 | 0.529476 | 0.46651 |
| | symmetric_UGGUGU-UGGAGU −6_3- | CAACUCCCUCGCGC | | CAGCCACA | | | | |
| | 3_bulge-symmetric_CAU-CAU | AACUUAGGAAAGUG | | ACTCCCTCG | | | | |
| | 0_1-1_mismatch_A-C | GGUUCAUGCAUAGA | | CGCAACTTA | | | | |
| | 6_1-1_mismatch_G-G | UCCACGGCUACAUA | | GGAAAGTG | | | | |
| | 10_1-1_mismatch_U-C | AUUCCUUUUGGAGU | | GGTTCATGC | | | | |
| | 17_3-3_bulge-symmetric_AGG-GGG | CACUGUCGUCGAAU | | ATAGATCC | | | | |
| | 26_1-2_bulge-asymmetric_A-AG | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 3 | −18_6-6_internal_loop- | CUCAGCAGCAGCCA | 442 | CTCAGCAG | 0.559995 | 0.559995 | 0.506012 | 0.449852 |
| | symmetric_UGGUGU-UGGAGU | CAACUCCCUCGCGC | | CAGCCACA | | | | |
| | −6_3-3_bulge-symmetric_CAU-CAU | AACUGGGAAAGGCU | | ACTCCCTCG | | | | |
| | 0_1-1_mismatch_A-C | UUCAUGCAUAGAUC | | CGCAACTG | | | | |
| | 6_1-1_mismatch_G-G | CACGGCUACAUAAU | | GGAAAGGC | | | | |
| | 10_1-1_mismatch_U-C | UCCUUUUGGAGUCA | | TTTCATGCA | | | | |
| | 19_2-1_bulge-asymmetric_GA-G | CUGUCGUCGAAUGG | | TAGATCCAC | | | | |
| | 26_2-2_bulge-symmetric_AA-GG | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 4 | −18_6-6_internal_loop- | CUCAGCAGCAGCCA | 443 | CTCAGCAG | 0.55193 | 0.55193 | 0.490742 | 0.428669 |
| | symmetric_UGGUGU-UGGAGU | CAACUCCCUCGCGC | | CAGCCACA | | | | |
| | −6_3-3_bulge-symmetric_CAU-CAU | AACUUCAAGUGGUU | | ACTCCCTCG | | | | |
| | 0_1-1_mismatch_A-C | UCAUGCAUAGAUCC | | CGCAACTTC | | | | |
| | 6_1-1_mismatch_G-G | ACGGCUACAUAAUU | | AAGTGGTTT | | | | |
| | 10_1-1_mismatch_U-C | CCUUUUGGAGUCAC | | CATGCATA | | | | |
| | 18_2-2_bulge-symmetric_GG-GG | UGUCGUCGAAUGGC | | GATCCACG | | | | |
| | 24_3-1_bulge-asymmetric_UCA-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 5 | −18_6-6_internal_loop- | CUCAGCAGCAGCCA | 444 | CTCAGCAG | 0.509767 | 0.509767 | 0.503348 | 0.446823 |
| | symmetric_UGGUGU-UGGAGU | CAACUCCCUCGCGC | | CAGCCACA | | | | |
| | −6_3-3_bulge-symmetric_CAU-CAU | AACGAAGAACAACC | | ACTCCCTCG | | | | |
| | 0_1-1_mismatch_A-C | UUUCAUGCAUAGAU | | CGCAACGA | | | | |
| | 6_1-1_mismatch_G-G | CCACGGCUACAUAA | | AGAACAAC | | | | |
| | 10_1-1_mismatch_U-C | UUCCUUUUGGAGUC | | CTTTCATGC | | | | |
| | 20_3-3_bulge-symmetric_ACU-CAA | ACUGUCGUCGAAUG | | ATAGATCC | | | | |
| | 26_3-3_bulge-symmetric_AAA-GAA | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 6 | −18_6-6_internal_loop- | CUCAGCAGCAGCCA | 445 | CTCAGCAG | 0.513521 | 0.513521 | 0.507167 | 0.424272 |
| | symmetric_UGGUGU-UGGAGU | CAACUCCCUCGCGC | | CAGCCACA | | | | |
| | −6_3-3_bulge-symmetric_CAU-CAU | AACUUCCGAAAGGA | | ACTCCCTCG | | | | |
| | 0_1-1_mismatch_A-C | CUUUCAUGCAUAGA | | CGCAACTTC | | | | |
| | 6_1-1_mismatch_G-G | UCCACGGCUACAUA | | CGAAAGGA | | | | |
| | 10_1-1_mismatch_U-C | AUUCCUUUUGGAGU | | CTTTCATGC | | | | |
| | 19_2-2_bulge-symmetric_GA-GA | CACUGUCGUCGAAU | | ATAGATCC | | | | |
| | 26_1-2_bulge-asymmetric_A-CC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 7 | −18_6-6_internal_loop- | CUCAGCAGCAGCCA | 446 | CTCAGCAG | 0.508293 | 0.508293 | 0.487919 | 0.44448 |
| | symmetric_UGGUGU-UGGAGU | CAACUCCCUCGCGC | | CAGCCACA | | | | |
| | −6_3-3_bulge-symmetric_CAU-CAU | AACGACGAAACAUC | | ACTCCCTCG | | | | |
| | 0_1-1_mismatch_A-C | CUUUCAUGCAUAGA | | CGCAACGA | | | | |
| | 6_1-1_mismatch_G-G | UCCACGGCUACAUA | | CGAAACAT | | | | |
| | 10_1-1_mismatch_U-C | AUUCCUUUUGGAGU | | CCTTTCATG | | | | |
| | 21_1-2_bulge-asymmetric_C-CA | CACUGUCGUCGAAU | | CATAGATCC | | | | |
| | 26_3-3_bulge-symmetric_AAA-GAC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 8 | −18_6-6_internal_loop- | CUCAGCAGCAGCCA | 447 | CTCAGCAG | 0.51058 | 0.51058 | 0.482622 | 0.422288 |
| | symmetric_UGGUGU-UGGAGU | CAACUCCCUCGCGC | | CAGCCACA | | | | |
| | −6_3-3_bulge-symmetric_CAU-CAU | AACGCGGAAAGCCC | | ACTCCCTCG | | | | |
| | 0_1-1_mismatch_A-C | CUUUCAUGCAUAGA | | CGCAACGC | | | | |
| | 6_1-1_mismatch_G-G | UCCACGGCUACAUA | | GGAAAGCC | | | | |
| | 10_1-1_mismatch_U-C | AUUCCUUUUGGAGU | | CCTTTCATG | | | | |
| | 20_1-2_bulge-asymmetric_A-CC | CACUGUCGUCGAAU | | CATAGATCC | | | | |
| | 26_3-3_bulge-symmetric_AAA-GCG | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 9 | −18_6-6_internal_loop- | CUCAGCAGCAGCCA | 448 | CTCAGCAG | 0.503242 | 0.503242 | 0.493603 | 0.431215 |
| | symmetric_UGGUGU-UGGAGU | CAACUCCCUCGCGC | | CAGCCACA | | | | |
| | −6_3-3_bulge-symmetric_CAU-CAU | AACUGCCAAAGAAC | | ACTCCCTCG | | | | |
| | 0_1-1_mismatch_A-C | UUUCAUGCAUAGAU | | CGCAACTG | | | | |
| | 6_1-1_mismatch_G-G | CCACGGCUACAUAA | | CCAAAGAA | | | | |
| | 10_1-1_mismatch_U-C | UUCCUUUUGGAGUC | | CTTTCATGC | | | | |
| | 19_2-2_bulge-symmetric_GA-AA | ACUGUCGUCGAAUG | | ATAGATCC | | | | |
| | 25_3-3_bulge-symmetric_CAA-GCC | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 10 | −18_6-6_internal_loop- | CUCAGCAGCAGCCA | 449 | CTCAGCAG | 0.508586 | 0.508586 | 0.461217 | 0.407601 |
| | symmetric_UGGUGU-UGGAGU | CAACUCCCUCGCGC | | CAGCCACA | | | | |
| | −6_3-3_bulge-symmetric_CAU-CAU | AACUUCGGAAAGCA | | ACTCCCTCG | | | | |
| | 0_1-1_mismatch_A-C | GCCUUUCAUGCAUA | | CGCAACTTC | | | | |
| | 6_1-1_mismatch_G-G | GAUCCACGGCUACA | | GGAAAGCA | | | | |
| | 10_1-1_mismatch_U-C | UAAUUCCUUUUGGA | | GCCTTTCAT | | | | |
| | 20_1-3_bulge-asymmetric_A-CAG | GUCACUGUCGUCGA | | GCATAGAT | | | | |
| | 26_1-2_bulge-asymmetric_A-CG | AUGGC | | CCACGGCT | | | | |
| | 30_6-6_internal_loop- | | | ACATAATTC | | | | |
| | symmetric_GCCAAG-GCGCAA | | | CTTTTGGAG | | | | |
| | | | | TCACTGTCG | | | | |
| | | | | TCGAATGG | | | | |
| | | | | C | | | | |
| 11 | −18_6-6_internal_loop- | CUCAGCAGCAGCCA | 450 | CTCAGCAG | 0.502825 | 0.502825 | 0.497874 | 0.41574 |
| | symmetric_UGGUGU-UGGAGU | CAACUCCCUCGCGC | | CAGCCACA | | | | |
| | −6_3-3_bulge-symmetric_CAU-CAU | AACAUUGAAAGUCG | | ACTCCCTCG | | | | |
| | 0_1-1_mismatch_A-C | UUCAUGCAUAGAUC | | CGCAACATT | | | | |
| | 6_1-1_mismatch_G-G | CACGGCUACAUAAU | | GAAAGTCG | | | | |
| | 10_1-1_mismatch_U-C | UCCUUUUGGAGUCA | | TTCATGCAT | | | | |
| | 17_2-1_bulge-asymmetric_AG-G | CUGUCGUCGAAUGG | | AGATCCAC | | | | |
| | 28_1-1_mismatch_A-A | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 12 | −18_6-6_internal_loop- | CUCAGCAGCAGCCA | 451 | CTCAGCAG | 0.545766 | 0.545766 | 0.480791 | 0.448235 |
| | symmetric_UGGUGU-UGGAGU | CAACUCCCUCGCGC | | CAGCCACA | | | | |
| | −6_3-3_bulge-symmetric_CAU-CAU | AACUUCGAAAGUGA | | ACTCCCTCG | | | | |
| | 0_1-1_mismatch_A-C | CUUCAUGCAUAGAU | | CGCAACTTC | | | | |
| | 6_1-1_mismatch_G-G | CCACGGCUACAUAA | | GAAAGTGA | | | | |
| | 10_1-1_mismatch_U-C | UUCCUUUUGGAGUC | | CTTCATGCA | | | | |
| | 17_3-3_bulge-symmetric_AGG-GAC | ACUGUCGUCGAAUG | | TAGATCCAC | | | | |
| | 26_1-1_mismatch_A-C | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 13 | −18_6-6_internal_loop- | CUCAGCAGCAGCCA | 452 | CTCAGCAG | 0.494338 | 0.494338 | 0.490024 | 0.436344 |
| | symmetric_UGGUGU-UGGAGU | CAACUCCCUCGCGC | | CAGCCACA | | | | |
| | −6_3-3_bulge-symmetric_CAU-CAU | AACGAUGAUUCCUU | | ACTCCCTCG | | | | |
| | 0_1-1_mismatch_A-C | UCAUGCAUAGAUCC | | CGCAACGA | | | | |
| | 6_1-1_mismatch_G-G | ACGGCUACAUAAUU | | TGATTCCTT | | | | |
| | 10_1-1_mismatch_U-C | CCUUUUGGAGUCAC | | TCATGCATA | | | | |
| | 21_3-1_bulge-asymmetric_CUU-U | UGUCGUCGAAUGGC | | GATCCACG | | | | |
| | 27_2-2_bulge-symmetric_AA-GA | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 14 | −18_6-6_internal_loop- | CUCAGCAGCAGCCA | 453 | CTCAGCAG | 0.496073 | 0.496073 | 0.47971 | 0.432824 |
| | symmetric_UGGUGU-UGGAGU | CAACUCCCUCGCGC | | CAGCCACA | | | | |
| | −6_3-3_bulge-symmetric_CAU-CAU | AACUGCAAAAGUCC | | ACTCCCTCG | | | | |
| | 0_1-1_mismatch_A-C | AUCAUGCAUAGAUC | | CGCAACTG | | | | |
| | 6_1-1_mismatch_G-G | CACGGCUACAUAAU | | CAAAAGTC | | | | |
| | 10_1-1_mismatch_U-C | UCCUUUUGGAGUCA | | CATCATGCA | | | | |
| | 16_2-1_bulge-asymmetric_AA-A | CUGUCGUCGAAUGG | | TAGATCCAC | | | | |
| | 25_3-3_bulge-symmetric_CAA-GCA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 15 | −18_6-6_internal_loop- | CUCAGCAGCAGCCA | 454 | CTCAGCAG | 0.507624 | 0.507624 | 0.470947 | 0.439491 |
| | symmetric_UGGUGU-UGGAGU | CAACUCCCUCGCGC | | CAGCCACA | | | | |
| | −6_3-3_bulge-symmetric_CAU-CAU | AACUUUGAACCGUC | | ACTCCCTCG | | | | |
| | 0_1-1_mismatch_A-C | CUUCCAUGCAUAGA | | CGCAACTTT | | | | |
| | 6_1-1_mismatch_G-G | UCCACGGCUACAUA | | GAACCGTC | | | | |
| | 10_1-1_mismatch_U-C | AUUCCUUUUGGAGU | | CTTCCATGC | | | | |
| | 15_1-1_mismatch_A-C | CACUGUCGUCGAAU | | ATAGATCC | | | | |
| | 22_1-2_bulge-asymmetric_U-CC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 16 | −18_6-6_internal_loop- | CUCAGCAGCAGCCA | 455 | CTCAGCAG | 0.538755 | 0.538755 | 0.48509 | 0.431374 |
| | symmetric_UGGUGU-UGGAGU | CAACUCCCUCGCGC | | CAGCCACA | | | | |
| | −6_3-3_bulge-symmetric_CAU-CAU | AACUUUGAUGUGAU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GATGTGATT | | | | |
| | 10_1-1_mismatch_U-C | UCCUUUUGGAGUCA | | TCATGCATA | | | | |
| | 18_2-2_bulge-symmetric_GG-GA | CUGUCGUCGAAUGG | | GATCCACG | | | | |
| | 22_2-1_bulge-asymmetric_UU-U | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 17 | −18_6-6_internal_loop- | CUCAGCAGCAGCCA | 456 | CTCAGCAG | 0.502795 | 0.502795 | 0.470986 | 0.423762 |
| | symmetric_UGGUGU-UGGAGU | CAACUCCCUCGCGC | | CAGCCACA | | | | |
| | −6_3-3_bulge-symmetric_CAU-CAU | AACUCCUGAAAGUG | | ACTCCCTCG | | | | |
| | 0_1-1_mismatch_A-C | UUCAUGCAUAGAUC | | CGCAACTCC | | | | |
| | 6_1-1_mismatch_G-G | CACGGCUACAUAAU | | TGAAAGTG | | | | |
| | 10_1-1_mismatch_U-C | UCCUUUUGGAGUCA | | TTCATGCAT | | | | |
| | 17_3-1_bulge-asymmetric_AGG-G | CUGUCGUCGAAUGG | | AGATCCAC | | | | |
| | 27_1-2_bulge-asymmetric_A-CC | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 18 | −18_6-6_internal_loop- | CUCAGCAGCAGCCA | 457 | CTCAGCAG | 0.495002 | 0.495002 | 0.478373 | 0.439865 |
| | symmetric_UGGUGU-UGGAGU | CAACUCCCUCGCGC | | CAGCCACA | | | | |
| | −6_3-3_bulge-symmetric_CAU-CAU | AACUUUACAAAAGU | | ACTCCCTCG | | | | |
| | 0_1-1_mismatch_A-C | GUUCAUGCAUAGAU | | CGCAACTTT | | | | |
| | 6_1-1_mismatch_G-G | CCACGGCUACAUAA | | ACAAAAGT | | | | |
| | 10_1-1_mismatch_U-C | UUCCUUUUGGAGUC | | GTTCATGCA | | | | |
| | 17_3-1_bulge-asymmetric_AGG-G | ACUGUCGUCGAAUG | | TAGATCCAC | | | | |
| | 25_1-3_bulge-asymmetric_C-ACA | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 19 | −18_6-6_internal_loop- | CUCAGCAGCAGCCA | 458 | CTCAGCAG | 0.525503 | 0.525503 | 0.473067 | 0.432093 |
| | symmetric_UGGUGU-UGGAGU | CAACUCCCUCGCGC | | CAGCCACA | | | | |
| | −6_3-3_bulge-symmetric_CAU-CAU | AACUUUGACGUAGU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GACGTAGTT | | | | |
| | 10_1-1_mismatch_U-C | UCCUUUUGGAGUCA | | TCATGCATA | | | | |
| | 18_2-2_bulge-symmetric_GG-AG | CUGUCGUCGAAUGG | | GATCCACG | | | | |
| | 22_2-1_bulge-asymmetric_UU-C | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 20 | −18_6-6_internal_loop- | CUCAGCAGCAGCCA | 459 | CTCAGCAG | 0.494962 | 0.494962 | 0.470346 | 0.427208 |
| | symmetric_UGGUGU-UGGAGU | CAACUCCCUCGCGC | | CAGCCACA | | | | |
| | −6_3-3_bulge-symmetric_CAU-CAU | AACGGUGAAAGUCA | | ACTCCCTCG | | | | |
| | 0_1-1_mismatch_A-C | AUUCAUGCAUAGAU | | CGCAACGG | | | | |
| | 6_1-1_mismatch_G-G | CCACGGCUACAUAA | | TGAAAGTC | | | | |
| | 10_1-1_mismatch_U-C | UUCCUUUUGGAGUC | | AATTCATGC | | | | |
| | 17_2-2_bulge-symmetric_AG-AA | ACUGUCGUCGAAUG | | ATAGATCC | | | | |
| | 27_2-2_bulge-symmetric_AA-GG | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 21 | −18_6-6_internal_loop- | CUCAGCAGCAGCCA | 460 | CTCAGCAG | 0.487994 | 0.487994 | 0.493899 | 0.43447 |
| | symmetric_UGGUGU-UGGAGU | CAACUCCCUCGCGC | | CAGCCACA | | | | |
| | -6_3-3_bulge-symmetric_CAU-CAU | AACUGCGACGCUCC | | ACTCCCTCG | | | | |
| | 0_1-1_mismatch_A-C | UUUCAUGCAUAGAU | | CGCAACTG | | | | |
| | 6_1-1_mismatch_G-G | CCACGGCUACAUAA | | CGACGCTCC | | | | |
| | 10_1-1_mismatch_U-C | UUCCUUUUGGAGUC | | TTTCATGCA | | | | |
| | 21_3-3_bulge-symmetric_CUU-CGC | ACUGUCGUCGAAUG | | TAGATCCAC | | | | |
| | 26_2-2_bulge-symmetric_AA-GC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 22 | −18_6-6_internal_loop- | CUCAGCAGCAGCCA | 461 | CTCAGCAG | 0.496075 | 0.496075 | 0.471822 | 0.425736 |
| | symmetric_UGGUGU-UGGAGU | CAACUCCCUCGCGC | | CAGCCACA | | | | |
| | -6_3-3_bulge-symmetric_CAU-CAU | AACUUUGCUAAGUC | | ACTCCCTCG | | | | |
| | 0_1-1_mismatch_A-C | AUCAUGCAUAGAUC | | CGCAACTTT | | | | |
| | 6_1-1_mismatch_G-G | CACGGCUACAUAAU | | GCTAAGTC | | | | |
| | 10_1-1_mismatch_U-C | UCCUUUUGGAGUCA | | ATCATGCAT | | | | |
| | 16_3-1_bulge-asymmetric_AAG-A | CUGUCGUCGAAUGG | | AGATCCAC | | | | |
| | 24_1-2_bulge-asymmetric_U-CU | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 23 | −18_6-6_internal_loop- | CUCAGCAGCAGCCA | 462 | CTCAGCAG | 0.498928 | 0.498928 | 0.483145 | 0.410751 |
| | symmetric_UGGUGU-UGGAGU | CAACUCCCUCGCGC | | CAGCCACA | | | | |
| | -6_3-3_bulge-symmetric_CAU-CAU | AACUGUGAAAGUGA | | ACTCCCTCG | | | | |
| | 0_1-1_mismatch_A-C | UUUCAUGCAUAGAU | | CGCAACTGT | | | | |
| | 6_1-1_mismatch_G-G | CCACGGCUACAUAA | | GAAAGTGA | | | | |
| | 10_1-1_mismatch_U-C | UUCCUUUUGGAGUC | | TTTCATGCA | | | | |
| | 18_2-2_bulge-symmetric_GG-GA | ACUGUCGUCGAAUG | | TAGATCCAC | | | | |
| | 27_1-1_mismatch_A-G | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 24 | −18_6-6_internal_loop- | CUCAGCAGCAGCCA | 463 | CTCAGCAG | 0.496184 | 0.496184 | 0.482306 | 0.408682 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGG | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GAAAGTGG | | | | |
| | 10_1-1_mismatch_U-C | UUCCUUUUGGAGUC | | TTTCATGCA | | | | |
| | 18_2-2_bulge-symmetric_GG-GG | ACUGUCGUCGAAUG | | TAGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 25 | −18_6-6_internal_loop- | CUCAGCAGCAGCCA | 464 | CTCAGCAG | 0.489731 | 0.489731 | 0.498726 | 0.420163 |
| | symmetric_UGGUGU-UGGAGU | CAACUCCCUCGCGC | | CAGCCACA | | | | |
| | -6_3-3_bulge-symmetric_CAU-CAU | AACUUUGCACAAGU | | ACTCCCTCG | | | | |
| | 0_1-1_mismatch_A-C | CGUUCAUGCAUAGA | | CGCAACTTT | | | | |
| | 6_1-1_mismatch_G-G | UCCACGGCUACAUA | | GCACAAGT | | | | |
| | 10_1-1_mismatch_U-C | AUUCCUUUUGGAGU | | CGTTCATGC | | | | |
| | 17_2-1_bulge-asymmetric_AG-G | CACUGUCGUCGAAU | | ATAGATCC | | | | |
| | 24_1-3_bulge-asymmetric_U-CAC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 26 | −18_6-6_internal_loop- | CUCAGCAGCAGCCA | 465 | CTCAGCAG | 0.523909 | 0.523909 | 0.492842 | 0.40553 |
| | symmetric_UGGUGU-UGGAGU | CAACUCCCUCGCGC | | CAGCCACA | | | | |
| | -6_3-3_bulge-symmetric_CAU-CAU | AACUGAGAAACCCC | | ACTCCCTCG | | | | |
| | 0_1-1_mismatch_A-C | UUUCAUGCAUAGAU | | CGCAACTG | | | | |
| | 6_1-1_mismatch_G-G | CCACGGCUACAUAA | | AGAAACCC | | | | |
| | 10_1-1_mismatch_U-C | UUCCUUUUGGAGUC | | CTTTCATGC | | | | |
| | 20_2-2_bulge-symmetric_AC-CC | ACUGUCGUCGAAUG | | ATAGATCC | | | | |
| | 26_2-2_bulge-symmetric_AA-GA | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 27 | −18_6-6_internal_loop- | CUCAGCAGCAGCCA | 466 | CTCAGCAG | 0.529123 | 0.529123 | 0.470213 | 0.440508 |
| | symmetric_UGGUGU-UGGAGU | CAACUCCCUCGCGC | | CAGCCACA | | | | |
| | -6_3-3_bulge-symmetric_CAU-CAU | AACUUUGAUCUAGU | | ACTCCCTCG | | | | |
| | 0_1-1_mismatch_A-C | GACUUUCAUGCAUA | | CGCAACTTT | | | | |
| | 6_1-1_mismatch_G-G | GAUCCACGGCUACA | | GATCTAGTG | | | | |
| | 10_1-1_mismatch_U-C | UAAUUCCUUUUGGA | | ACTTTCATG | | | | |
| | 19_1-2_bulge-asymmetric_G-GA | GUCACUGUCGUCGA | | CATAGATCC | | | | |
| | 23_1-3_bulge-asymmetric_U-UCU | AUGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 28 | −18_6-6_internal_loop- | CUCAGCAGCAGCCA | 467 | CTCAGCAG | 0.485493 | 0.485493 | 0.469274 | 0.429259 |
| | symmetric_UGGUGU-UGGAGU | CAACUCCCUCGCGC | | CAGCCACA | | | | |
| | -6_3-3_bulge-symmetric_CAU-CAU | AACGGGGAAAGUCC | | ACTCCCTCG | | | | |
| | 0_1-1_mismatch_A-C | UUCCAUGCAUAGAU | | CGCAACGG | | | | |
| | 6_1-1_mismatch_G-G | CCACGGCUACAUAA | | GGAAAGTC | | | | |
| | 10_1-1_mismatch_U-C | UUCCUUUUGGAGUC | | CTTCCATGC | | | | |
| | 15_1-1_mismatch_A-C | ACUGUCGUCGAAUG | | ATAGATCC | | | | |
| | 26_3-3_bulge-symmetric_AAA-GGG | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 29 | −18_6-6_internal_loop- | CUCAGCAGCAGCCA | 468 | CTCAGCAG | 0.489591 | 0.489591 | 0.498918 | 0.421443 |
| | symmetric_UGGUGU-UGGAGU | CAACUCCCUCGCGC | | CAGCCACA | | | | |
| | -6_3-3_bulge-symmetric_CAU-CAU | AACUUUGAUCGUCG | | ACTCCCTCG | | | | |
| | 0_1-1_mismatch_A-C | GUUCAUGCAUAGAU | | CGCAACTTT | | | | |
| | 6_1-1_mismatch_G-G | CCACGGCUACAUAA | | GATCGTCG | | | | |
| | 10_1-1_mismatch_U-C | UUCCUUUUGGAGUC | | GTTCATGCA | | | | |
| | 17_2-2_bulge-symmetric_AG-GG | ACUGUCGUCGAAUG | | TAGATCCAC | | | | |
| | 22_2-2_bulge-symmetric_UU-UC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 30 | −18_6-6_internal_loop- | CUCAGCAGCAGCCA | 469 | CTCAGCAG | 0.490403 | 0.490403 | 0.465221 | 0.442358 |
| | symmetric_UGGUGU-UGGAGU | CAACUCCCUCGCGC | | CAGCCACA | | | | |
| | -6_3-3_bulge-symmetric_CAU-CAU | AACUCAUGAUCCUC | | ACTCCCTCG | | | | |
| | 0_1-1_mismatch_A-C | CUUUCAUGCAUAGA | | CGCAACTC | | | | |
| | 6_1-1_mismatch_G-G | UCCACGGCUACAUA | | ATGATCCTC | | | | |
| | 10_1-1_mismatch_U-C | AUUCCUUUUGGAGU | | CTTTCATGC | | | | |
| | 21_3-3_bulge-symmetric_CUU-UCC | CACUGUCGUCGAAU | | ATAGATCC | | | | |
| | 27_1-2_bulge-asymmetric_A-CA | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 31 | −18_6-6_internal_loop- | CUCAGCAGCAGCCA | 470 | CTCAGCAG | 0.500024 | 0.500024 | 0.470777 | 0.402047 |
| | symmetric_UGGUGU-UGGAGU | CAACUCCCUCGCGC | | CAGCCACA | | | | |
| | -6_3-3_bulge-symmetric_CAU-CAU | AACUUUGACCCUCC | | ACTCCCTCG | | | | |
| | 0_1-1_mismatch_A-C | UUUCAUGCAUAGAA | | CGCAACTTT | | | | |
| | 4_1-1_mismatch_A-A | CCACGGCUACAUAA | | GACCCTCCT | | | | |
| | 6_1-1_mismatch_G-G | UUCCUUUUGGAGUC | | TTCATGCAT | | | | |
| | 10_1-1_mismatch_U-C | ACUGUCGUCGAAUG | | AGAACCAC | | | | |
| | 21_3-3_bulge-symmetric_CUU-CCC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 32 | −18_6-6_internal_loop- | CUCAGCAGCAGCCA | 471 | CTCAGCAG | 0.508365 | 0.508365 | 0.466032 | 0.430517 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGA | | ACTCCCTCG | | | | |
| | 0_1-1_mismatch_A-C | CUUCAUGCAUAGAU | | CGCAACTTT | | | | |
| | 6_1-1_mismatch_G-G | CCACGGCUACAUAA | | GAAAGTGA | | | | |
| | 10_1-1_mismatch_U-C | UUCCUUUUGGAGUC | | CTTCATGCA | | | | |
| | 17_3-3_bulge-symmetric_AGG-GAC | ACUGUCGUCGAAUG | | TAGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 33 | −18_6-6_internal_loop- | CUCAGCAGCAGCCA | 472 | CTCAGCAG | 0.488348 | 0.488348 | 0.49385 | 0.407763 |
| | symmetric_UGGUGU-UGGAGU | CAACUCCCUCGCGC | | CAGCCACA | | | | |
| | -6_3-3_bulge-symmetric_CAU-CAU | AACUUUGUCCGGGC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GTCCGGGCT | | | | |
| | 10_1-1_mismatch_U-C | UUCCUUUUGGAGUC | | TTCATGCAT | | | | |
| | 19_2-2_bulge-symmetric_GA-GG | ACUGUCGUCGAAUG | | AGATCCAC | | | | |
| | 22_3-3_bulge-symmetric_UUU-UCC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 34 | −18_6-6_internal_loop- | CUCAGCAGCAGCCA | 473 | CTCAGCAG | 0.516321 | 0.516321 | 0.464419 | 0.419123 |
| | symmetric_UGGUGU-UGGAGU | CAACUCCCUCGCGC | | CAGCCACA | | | | |
| | -6_3-3_bulge-symmetric_CAU-CAU | AACUUCAUAAGUCC | | ACTCCCTCG | | | | |
| | 0_1-1_mismatch_A-C | GGGCAUGCAUAGAU | | CGCAACTTC | | | | |
| | 6_1-1_mismatch_G-G | CCACGGCUACAUAA | | ATAAGTCC | | | | |
| | 10_1-1_mismatch_U-C | UUCCUUUUGGAGUC | | GGGCATGC | | | | |
| | 15_3-3_bulge-symmetric_AAA-GGG | ACUGUCGUCGAAUG | | ATAGATCC | | | | |
| | 24_3-3_bulge-symmetric_UCA-CAU | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 35 | −18_6-6_internal_loop- | CUCAGCAGCAGCCA | 474 | CTCAGCAG | 0.48425 | 0.48425 | 0.475435 | 0.426979 |
| | symmetric_UGGUGU-UGGAGU | CAACUCCCUCGCGC | | CAGCCACA | | | | |
| | -6_3-3_bulge-symmetric_CAU-CAU | AACUCAUGAUGUCC | | ACTCCCTCG | | | | |
| | 0_1-1_mismatch_A-C | UUUCAUGCAUAGAU | | CGCAACTC | | | | |
| | 6_1-1_mismatch_G-G | CCACGGCUACAUAA | | ATGATGTCC | | | | |
| | 10_1-1_mismatch_U-C | UUCCUUUUGGAGUC | | TTTCATGCA | | | | |
| | 22_2-1_bulge-asymmetric_UU-U | ACUGUCGUCGAAUG | | TAGATCCAC | | | | |
| | 27_1-2_bulge-asymmetric_A-CA | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 36 | −18_6-6_internal_loop- | CUCAGCAGCAGCCA | 475 | CTCAGCAG | 0.484894 | 0.484894 | 0.474018 | 0.410959 |
| | symmetric_UGGUGU-UGGAGU | CAACUCCCUCGCGC | | CAGCCACA | | | | |
| | -6_3-3_bulge-symmetric_CAU-CAU | AACUUUGUUAGUCC | | ACTCCCTCG | | | | |
| | 0_1-1_mismatch_A-C | GUCAUGCAUAGAUC | | CGCAACTTT | | | | |
| | 6_1-1_mismatch_G-G | CACGGCUACAUAAU | | GTTAGTCCG | | | | |
| | 10_1-1_mismatch_U-C | UCCUUUUGGAGUCA | | TCATGCATA | | | | |
| | 16_2-1_bulge-asymmetric_AA-G | CUGUCGUCGAAUGG | | GATCCACG | | | | |
| | 23_2-2_bulge-symmetric_UU-UU | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 37 | −18_6-6_internal_loop- | CUCAGCAGCAGCCA | 476 | CTCAGCAG | 0.50368 | 0.50368 | 0.475875 | 0.419977 |
| | symmetric_UGGUGU-UGGAGU | CAACUCCCUCGCGC | | CAGCCACA | | | | |
| | -6_3-3_bulge-symmetric_CAU-CAU | AACUUAUCAAGUCG | | ACTCCCTCG | | | | |
| | 0_1-1_mismatch_A-C | GAUCAUGCAUAGAU | | CGCAACTTA | | | | |
| | 6_1-1_mismatch_G-G | CCACGGCUACAUAA | | TCAAGTCG | | | | |
| | 10_1-1_mismatch_U-C | UUCCUUUUGGAGUC | | GATCATGC | | | | |
| | 16_3-3_bulge-symmetric_AAG-GGA | ACUGUCGUCGAAUG | | ATAGATCC | | | | |
| | 24_3-3_bulge-symmetric_UCA-AUC | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 38 | −18_6-6_internal_loop- | CUCAGCAGCAGCCA | 477 | CTCAGCAG | 0.481279 | 0.481279 | 0.490273 | 0.418624 |
| | symmetric_UGGUGU-UGGAGU | CAACUCCCUCGCGC | | CAGCCACA | | | | |
| | -6_3-3_bulge-symmetric_CAU-CAU | AACUUCAAGUCAGU | | ACTCCCTCG | | | | |
| | 0_1-1_mismatch_A-C | UCAUGCAUAGAUCC | | CGCAACTTC | | | | |
| | 6_1-1_mismatch_G-G | ACGGCUACAUAAUU | | AAGTCAGTT | | | | |
| | 10_1-1_mismatch_U-C | CCUUUUGGAGUCAC | | CATGCATA | | | | |
| | 17_2-2_bulge-symmetric_AG-AG | UGUCGUCGAAUGGC | | GATCCACG | | | | |
| | 24_3-1_bulge-asymmetric_UCA-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 39 | −18_6-6_internal_loop- | CUCAGCAGCAGCCA | 478 | CTCAGCAG | 0.508315 | 0.508315 | 0.445698 | 0.409205 |
| | symmetric_UGGUGU-UGGAGU | CAACUCCCUCGCGC | | CAGCCACA | | | | |
| | -6_3-3_bulge-symmetric_CAU-CAU | AACUUUCAGUCCUU | | ACTCCCTCG | | | | |
| | 0_1-1_mismatch_A-C | UGAUGCAUAGAUCC | | CGCAACTTT | | | | |
| | 6_1-1_mismatch_G-G | ACGGCUACAUAAUU | | CAGTCCTTT | | | | |
| | 10_1-1_mismatch_U-C | CCUUUUGGAGUCAC | | GATGCATA | | | | |
| | 14_1-1_mismatch_G-G | UGUCGUCGAAUGGC | | GATCCACG | | | | |
| | 23_3-1_bulge-asymmetric_UUC-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 40 | −18_6-6_internal_loop- | CUCAGCAGCAGCCA | 479 | CTCAGCAG | 0.484544 | 0.484544 | 0.467506 | 0.407246 |
| | symmetric_UGGUGU-UGGAGU | CAACUCCCUCGCGC | | CAGCCACA | | | | |
| | -6_3-3_bulge-symmetric_CAU-CAU | AACUUUGUAAGUCA | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GTAAGTCAT | | | | |
| | 10_1-1_mismatch_U-C | UCCUUUUGGAGUCA | | TCATGCATA | | | | |
| | 17_2-1_bulge-asymmetric_AG-A | CUGUCGUCGAAUGG | | GATCCACG | | | | |
| | 24_1-1_mismatch_U-U | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 41 | −18_6-6_internal_loop- | CUCAGCAGCAGCCA | 480 | CTCAGCAG | 0.493226 | 0.493226 | 0.450952 | 0.419307 |
| | symmetric_UGGUGU-UGGAGU | CAACUCCCUCGCGC | | CAGCCACA | | | | |
| | -6_3-3_bulge-symmetric_CAU-CAU | AACUUUGCGUCGUC | | ACTCCCTCG | | | | |
| | 0_1-1_mismatch_A-C | AUGCAUAGAUCCAC | | CGCAACTTT | | | | |
| | 6_1-1_mismatch_G-G | GGCUACAUAAUUCC | | GCGTCGTCA | | | | |
| | 10_1-1_mismatch_U-C | UUUUGGAGUCACUG | | TGCATAGAT | | | | |
| | 16_3-1_bulge-asymmetric_AAG-G | UCGUCGAAUGGC | | CCACGGCT | | | | |
| | 22_3-1_bulge-asymmetric_UUU-C | | | ACATAATTC | | | | |
| | 30_6-6_internal_loop- | | | CTTTTGGAG | | | | |
| | symmetric_GCCAAG-GCGCAA | | | TCACTGTCG | | | | |
| | | | | TCGAATGG | | | | |
| | | | | C | | | | |
| 42 | −18_6-6_internal_loop- | CUCAGCAGCAGCCA | 481 | CTCAGCAG | 0.498399 | 0.498399 | 0.470599 | 0.410077 |
| | symmetric_UGGUGU-UGGAGU | CAACUCCCUCGCGC | | CAGCCACA | | | | |
| | -6_3-3_bulge-symmetric_CAU-CAU | AACUUGUGGAAAGU | | ACTCCCTCG | | | | |
| | 0_1-1_mismatch_A-C | CGAUUCAUGCAUAG | | CGCAACTTG | | | | |
| | 6_1-1_mismatch_G-G | AUCCACGGCUACAU | | TGGAAAGT | | | | |
| | 10_1-1_mismatch_U-C | AAUUCCUUUUGGAG | | CGATTCATG | | | | |
| | 17_2-2_bulge-symmetric_AG-GA | UCACUGUCGUCGAA | | CATAGATCC | | | | |
| | 26_1-3_bulge-asymmetric_A-GUG | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 43 | −18_6-6_internal_loop- | CUCAGCAGCAGCCA | 482 | CTCAGCAG | 0.493035 | 0.493035 | 0.473217 | 0.427973 |
| | symmetric_UGGUGU-UGGAGU | CAACUCCCUCGCGC | | CAGCCACA | | | | |
| | -6_3-3_bulge-symmetric_CAU-CAU | AACUUUGAUGUCGU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GATGTCGTT | | | | |
| | 10_1-1_mismatch_U-C | CCUUUUGGAGUCAC | | CATGCATA | | | | |
| | 17_2-1_bulge-asymmetric_AG-G | UGUCGUCGAAUGGC | | GATCCACG | | | | |
| | 22_2-1_bulge-asymmetric_UU-U | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 44 | −18_6-6_internal_loop- | CUCAGCAGCAGCCA | 483 | CTCAGCAG | 0.477501 | 0.477501 | 0.477777 | 0.404957 |
| | symmetric_UGGUGU-UGGAGU | CAACUCCCUCGCGC | | CAGCCACA | | | | |
| | -6_3-3_bulge-symmetric_CAU-CAU | AACGAUGAAAUCUU | | ACTCCCTCG | | | | |
| | 0_1-1_mismatch_A-C | CCUUUCAUGCAUAG | | CGCAACGA | | | | |
| | 6_1-1_mismatch_G-G | AUCCACGGCUACAU | | TGAAATCTT | | | | |
| | 10_1-1_mismatch_U-C | AAUUCCUUUUGGAG | | CCTTTCATG | | | | |
| | 21_1-3_bulge-asymmetric_C-UCU | UCACUGUCGUCGAA | | CATAGATCC | | | | |
| | 27_2-2_bulge-symmetric_AA-GA | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 45 | −18_6-6_internal_loop- | CUCAGCAGCAGCCA | 484 | CTCAGCAG | 0.483106 | 0.483106 | 0.485834 | 0.398475 |
| | symmetric_UGGUGU-UGGAGU | CAACUCCCUCGCGC | | CAGCCACA | | | | |
| | -6_3-3_bulge-symmetric_CAU-CAU | AACGGUGAAAGUCC | | ACTCCCTCG | | | | |
| | 0_1-1_mismatch_A-C | UUUCAUGCAUAGAU | | CGCAACGG | | | | |
| | 2_1-1_wobble_G-U | CUACGGCUACAUAA | | TGAAAGTC | | | | |
| | 6_1-1_mismatch_G-G | UUCCUUUUGGAGUC | | CTTTCATGC | | | | |
| | 10_1-1_mismatch_U-C | ACUGUCGUCGAAUG | | ATAGATCTA | | | | |
| | 27_2-2_bulge-symmetric_AA-GG | GC | | CGGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 46 | −18_6-6_internal_loop- | CUCAGCAGCAGCCA | 485 | CTCAGCAG | 0.47459 | 0.47459 | 0.468031 | 0.415703 |
| | symmetric_UGGUGU-UGGAGU | CAACUCCCUCGCGC | | CAGCCACA | | | | |
| | -6_3-3_bulge-symmetric_CAU-CAU | AACUUAUAGAAAGU | | ACTCCCTCG | | | | |
| | 0_1-1_mismatch_A-C | CCUGCAUGCAUAGA | | CGCAACTTA | | | | |
| | 6_1-1_mismatch_G-G | UCCACGGCUACAUA | | TAGAAAGT | | | | |
| | 10_1-1_mismatch_U-C | AUUCCUUUUGGAGU | | CCTGCATGC | | | | |
| | 15_2-1_bulge-asymmetric_AA-G | CACUGUCGUCGAAU | | ATAGATCC | | | | |
| | 26_1-3_bulge-asymmetric_A-AUA | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 47 | −18_6-6_internal_loop- | CUCAGCAGCAGCCA | 486 | CTCAGCAG | 0.477726 | 0.477726 | 0.48897 | 0.417785 |
| | symmetric_UGGUGU-UGGAGU | CAACUCCCUCGCGC | | CAGCCACA | | | | |
| | -6_3-3_bulge-symmetric_CAU-CAU | AACUUUGAAAGGCU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GAAAGGCT | | | | |
| | 10_1-1_mismatch_U-C | UCCUUUUGGAGUCA | | TTCATGCAT | | | | |
| | 19_2-1_bulge-asymmetric_GA-G | CUGUCGUCGAAUGG | | AGATCCAC | | | | |
| | 30_6-6_internal_loop- | C | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 48 | −18_6-6_internal_loop- | CUCAGCAGCAGCCA | 487 | CTCAGCAG | 0.473683 | 0.473683 | 0.470568 | 0.430347 |
| | symmetric_UGGUGU-UGGAGU | CAACUCCCUCGCGC | | CAGCCACA | | | | |
| | -6_3-3_bulge-symmetric_CAU-CAU | AACUCCAAAACGCC | | ACTCCCTCG | | | | |
| | 0_1-1_mismatch_A-C | UUUCAUGCAUAGAU | | CGCAACTCC | | | | |
| | 6_1-1_mismatch_G-G | CCACGGCUACAUAA | | AAAACGCC | | | | |
| | 10_1-1_mismatch_U-C | UUCCUUUUGGAGUC | | TTTCATGCA | | | | |
| | 20_2-2_bulge-symmetric_AC-CG | ACUGUCGUCGAAUG | | TAGATCCAC | | | | |
| | 25_3-3_bulge-symmetric_CAA-CCA | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 49 | −18_6-6_internal_loop- | CUCAGCAGCAGCCA | 488 | CTCAGCAG | 0.47379 | 0.47379 | 0.468569 | 0.424309 |
| | symmetric_UGGUGU-UGGAGU | CAACUCCCUCGCGC | | CAGCCACA | | | | |
| | -6_3-3_bulge-symmetric_CAU-CAU | AACUUUCAAGUCAU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | CAAGTCATT | | | | |
| | 10_1-1_mismatch_U-C | CCUUUUGGAGUCAC | | CATGCATA | | | | |
| | 17_2-1_bulge-asymmetric_AG-A | UGUCGUCGAAUGGC | | GATCCACG | | | | |
| | 24_2-1_bulge-asymmetric_UC-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 50 | −18_6-6_internal_loop- | CUCAGCAGCAGCCA | 489 | CTCAGCAG | 0.471324 | 0.471324 | 0.482706 | 0.412427 |
| | symmetric_UGGUGU-UGGAGU | CAACUCCCUCGCGC | | CAGCCACA | | | | |
| | -6_3-3_bulge-symmetric_CAU-CAU | AACUUCAAAGUAAU | | ACTCCCTCG | | | | |
| | 0_1-1_mismatch_A-C | UUCAUGCAUAGAUC | | CGCAACTTC | | | | |
| | 6_1-1_mismatch_G-G | CACGGCUACAUAAU | | AAAGTAAT | | | | |
| | 10_1-1_mismatch_U-C | UCCUUUUGGAGUCA | | TTCATGCAT | | | | |
| | 18_2-2_bulge-symmetric_GG-AA | CUGUCGUCGAAUGG | | AGATCCAC | | | | |
| | 25_2-1_bulge-asymmetric_CA-C | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 51 | −18_6-6_internal_loop- | CUCAGCAGCAGCCA | 490 | CTCAGCAG | 0.504752 | 0.504752 | 0.490091 | 0.400307 |
| | symmetric_UGGUGU-UGGAGU | CAACUCCCUCGCGC | | CAGCCACA | | | | |
| | -6_3-3_bulge-symmetric_CAU-CAU | AACUUUACAAAAGC | | ACTCCCTCG | | | | |
| | 0_1-1_mismatch_A-C | ACUUUCAUGCAUAG | | CGCAACTTT | | | | |
| | 6_1-1_mismatch_G-G | AUCCACGGCUACAU | | ACAAAAGC | | | | |
| | 10_1-1_mismatch_U-C | AAUUCCUUUUGGAG | | ACTTTCATG | | | | |
| | 19_2-2_bulge-symmetric_GA-CA | UCACUGUCGUCGAA | | CATAGATCC | | | | |
| | 25_1-3_bulge-asymmetric_C-ACA | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 52 | −18_6-6_internal_loop- | CUCAGCAGCAGCCA | 491 | CTCAGCAG | 0.482272 | 0.482272 | 0.449263 | 0.391704 |
| | symmetric_UGGUGU-UGGAGU | CAACUCCCUCGCGC | | CAGCCACA | | | | |
| | -6_3-3_bulge-symmetric_CAU-CAU | AACGAAGAACUUCC | | ACTCCCTCG | | | | |
| | 0_1-1_mismatch_A-C | UUUCAUGCAUAGAU | | CGCAACGA | | | | |
| | 6_1-1_mismatch_G-G | CCACGGCUACAUAA | | AGAACTTCC | | | | |
| | 10_1-1_mismatch_U-C | UUCCUUUUGGAGUC | | TTTCATGCA | | | | |
| | 21_2-2_bulge-symmetric_CU-CU | ACUGUCGUCGAAUG | | TAGATCCAC | | | | |
| | 26_3-3_bulge-symmetric_AAA-GAA | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 53 | −18_6-6_internal_loop- | CUCAGCAGCAGCCA | 492 | CTCAGCAG | 0.46751 | 0.46751 | 0.482801 | 0.420663 |
| | symmetric_UGGUGU-UGGAGU | CAACUCCCUCGCGC | | CAGCCACA | | | | |
| | -6_3-3_bulge-symmetric_CAU-CAU | AACUUACAAACCUC | | ACTCCCTCG | | | | |
| | 0_1-1_mismatch_A-C | CUUUCAUGCAUAGA | | CGCAACTTA | | | | |
| | 6_1-1_mismatch_G-G | UCCACGGCUACAUA | | CAAACCTCC | | | | |
| | 10_1-1_mismatch_U-C | AUUCCUUUUGGAGU | | TTTCATGCA | | | | |
| | 21_1-2_bulge-asymmetric_C-CC | CACUGUCGUCGAAU | | TAGATCCAC | | | | |
| | 25_2-2_bulge-symmetric_CA-AC | GGC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 54 | −18_6-6_internal_loop- | CUCAGCAGCAGCCA | 493 | CTCAGCAG | 0.478937 | 0.478937 | 0.468718 | 0.411667 |
| | symmetric_UGGUGU-UGGAGU | CAACUCCCUCGCGC | | CAGCCACA | | | | |
| | -6_3-3_bulge-symmetric_CAU-CAU | AACAUUGACGUCCU | | ACTCCCTCG | | | | |
| | 0_1-1_mismatch_A-C | UUCAUGCAUAGAUC | | CGCAACATT | | | | |
| | 6_1-1_mismatch_G-G | CACGGCUACAUAAU | | GACGTCCTT | | | | |
| | 10_1-1_mismatch_U-C | UCCUUUUGGAGUCA | | TCATGCATA | | | | |
| | 22_2-1_bulge-asymmetric_UU-C | CUGUCGUCGAAUGG | | GATCCACG | | | | |
| | 28_1-1_mismatch_A-A | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 55 | −18_6-6_internal_loop- | CUCAGCAGCAGCCA | 494 | CTCAGCAG | 0.479888 | 0.479888 | 0.467238 | 0.413521 |
| | symmetric_UGGUGU-UGGAGU | CAACUCCCUCGCGC | | CAGCCACA | | | | |
| | -6_3-3_bulge-symmetric_CAU-CAU | AACGCUGAAAGUCC | | ACTCCCTCG | | | | |
| | 0_1-1_mismatch_A-C | GGGUUCAUGCAUAG | | CGCAACGC | | | | |
| | 6_1-1_mismatch_G-G | AUCCACGGCUACAU | | TGAAAGTC | | | | |
| | 10_1-1_mismatch_U-C | AAUUCCUUUUGGAG | | CGGGTTCAT | | | | |
| | 17_1-3_bulge-asymmetric_A-GGG | UCACUGUCGUCGAA | | GCATAGAT | | | | |
| | 27_2-2_bulge-symmetric_AA-GC | UGGC | | CCACGGCT | | | | |
| | 30_6-6_internal_loop- | | | ACATAATTC | | | | |
| | symmetric_GCCAAG-GCGCAA | | | CTTTTGGAG | | | | |
| | | | | TCACTGTCG | | | | |
| | | | | TCGAATGG | | | | |
| | | | | C | | | | |
| 56 | −18_6-6_internal_loop- | CUCAGCAGCAGCCA | 495 | CTCAGCAG | 0.524837 | 0.524837 | 0.473327 | 0.446365 |
| | symmetric_UGGUGU-UGGAGU | CAACUCCCUCGCGC | | CAGCCACA | | | | |
| | -6_3-3_bulge-symmetric_CAU-CAU | AACUUUGAAAGCAG | | ACTCCCTCG | | | | |
| | 0_1-1_mismatch_A-C | CAGUUCAUGCAUAG | | CGCAACTTT | | | | |
| | 6_1-1_mismatch_G-G | AUCCACGGCUACAU | | GAAAGCAG | | | | |
| | 10_1-1_mismatch_U-C | AAUUCCUUUUGGAG | | CAGTTCATG | | | | |
| | 17_2-2_bulge-symmetric_AG-AG | UCACUGUCGUCGAA | | CATAGATCC | | | | |
| | 20_1-3_bulge-asymmetric_A-CAG | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 57 | −18_6-6_internal_loop- | CUCAGCAGCAGCCA | 496 | CTCAGCAG | 0.46869 | 0.46869 | 0.473915 | 0.415134 |
| | symmetric_UGGUGU-UGGAGU | CAACUCCCUCGCGC | | CAGCCACA | | | | |
| | -6_3-3_bulge-symmetric_CAU-CAU | AACUUGCGAAAGUA | | ACTCCCTCG | | | | |
| | 0_1-1_mismatch_A-C | ACUUCAUGCAUAGA | | CGCAACTTG | | | | |
| | 6_1-1_mismatch_G-G | UCCACGGCUACAUA | | CGAAAGTA | | | | |
| | 10_1-1_mismatch_U-C | AUUCCUUUUGGAGU | | ACTTCATGC | | | | |
| | 17_3-3_bulge-symmetric_AGG-AAC | CACUGUCGUCGAAU | | ATAGATCC | | | | |
| | 26_1-2_bulge-asymmetric_A-GC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 58 | −18_6-6_internal_loop- | CUCAGCAGCAGCCA | 497 | CTCAGCAG | 0.497302 | 0.497302 | 0.463722 | 0.424582 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GAAAGTGT | | | | |
| | 10_1-1_mismatch_U-C | CCUUUUGGAGUCAC | | TCATGCATA | | | | |
| | 17_3-1_bulge-asymmetric_AGG-G | UGUCGUCGAAUGGC | | GATCCACG | | | | |
| | 30_6-6_internal_loop- | | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 59 | −18_6-6_internal_loop- | CUCAGCAGCAGCCA | 498 | CTCAGCAG | 0.481268 | 0.481268 | 0.46092 | 0.397179 |
| | symmetric_UGGUGU-UGGAGU | CAACUCCCUCGCGC | | CAGCCACA | | | | |
| | -6_3-3_bulge-symmetric_CAU-CAU | AACUUUGUGUCCUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GTGTCCTTT | | | | |
| | 10_1-1_mismatch_U-C | CCUUUUGGAGUCAC | | CATGCATA | | | | |
| | 22_3-1_bulge-asymmetric_UUU-U | UGUCGUCGAAUGGC | | GATCCACG | | | | |
| | 30_6-6_internal_loop- | | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 60 | −18_6-6_internal_loop- | CUCAGCAGCAGCCA | 499 | CTCAGCAG | 0.561258 | 0.561258 | 0.445955 | 0.435844 |
| | symmetric_UGGUGU-UGGAGU | CAACUCCCUCGCGC | | CAGCCACA | | | | |
| | -6_3-3_bulge-symmetric_CAU-CAU | AACUUCAAGUGGCU | | ACTCCCTCG | | | | |
| | 0_1-1_mismatch_A-C | UUCAUGCAUAGAUC | | CGCAACTTC | | | | |
| | 6_1-1_mismatch_G-G | CACGGCUACAUAAU | | AAGTGGCTT | | | | |
| | 10_1-1_mismatch_U-C | UCCUUUUGGAGUCA | | TCATGCATA | | | | |
| | 19_1-2_bulge-asymmetric_G-GG | CUGUCGUCGAAUGG | | GATCCACG | | | | |
| | 24_3-1_bulge-asymmetric_UCA-C | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 61 | −18_6-6_internal_loop- | CUCAGCAGCAGCCA | 500 | CTCAGCAG | 0.476532 | 0.476532 | 0.469477 | 0.405773 |
| | symmetric_UGGUGU-UGGAGU | CAACUCCCUCGCGC | | CAGCCACA | | | | |
| | -6_3-3_bulge-symmetric_CAU-CAU | AACUUUGAUUAGUC | | ACTCCCTCG | | | | |
| | 0_1-1_mismatch_A-C | CGAUUCAUGCAUAG | | CGCAACTTT | | | | |
| | 6_1-1_mismatch_G-G | AUCCACGGCUACAU | | GATTAGTCC | | | | |
| | 10_1-1_mismatch_U-C | AAUUCCUUUUGGAG | | GATTCATGC | | | | |
| | 17_1-2_bulge-asymmetric_A-GA | UCACUGUCGUCGAA | | ATAGATCC | | | | |
| | 23_1-2_bulge-asymmetric_U-UU | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 62 | −18_6-6_internal_loop- | CUCAGCAGCAGCCA | 501 | CTCAGCAG | 0.467735 | 0.467735 | 0.460755 | 0.406338 |
| | symmetric_UGGUGU-UGGAGU | CAACUCCCUCGCGC | | CAGCCACA | | | | |
| | -6_3-3_bulge-symmetric_CAU-CAU | AACUUCCCAAGUCC | | ACTCCCTCG | | | | |
| | 0_1-1_mismatch_A-C | UUUCAUGCAUAGAU | | CGCAACTTC | | | | |
| | 6_1-1_mismatch_G-G | CCACGGCUACAUAA | | CCAAGTCCT | | | | |
| | 10_1-1_mismatch_U-C | UUCCUUUUGGAGUC | | TTCATGCAT | | | | |
| | 24_3-3_bulge-symmetric_UCA-CCC | ACUGUCGUCGAAUG | | AGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 63 | −18_6-6_internal_loop- | CUCAGCAGCAGCCA | 502 | CTCAGCAG | 0.462366 | 0.462366 | 0.461661 | 0.407917 |
| | symmetric_UGGUGU-UGGAGU | CAACUCCCUCGCGC | | CAGCCACA | | | | |
| | -6_3-3_bulge-symmetric_CAU-CAU | AACAUUGUCCAAGU | | ACTCCCTCG | | | | |
| | 0_1-1_mismatch_A-C | CCUUUCAUGCAUAG | | CGCAACATT | | | | |
| | 6_1-1_mismatch_G-G | AUCCACGGCUACAU | | GTCCAAGTC | | | | |
| | 10_1-1_mismatch_U-C | AAUUCCUUUUGGAG | | CTTTCATGC | | | | |
| | 24_1-3_bulge-asymmetric_U-UCC | UCACUGUCGUCGAA | | ATAGATCC | | | | |
| | 28_1-1_mismatch_A-A | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 64 | −18_6-6_internal_loop- | CUCAGCAGCAGCCA | 503 | CTCAGCAG | 0.476081 | 0.476081 | 0.462984 | 0.431412 |
| | symmetric_UGGUGU-UGGAGU | CAACUCCCUCGCGC | | CAGCCACA | | | | |
| | -6_3-3_bulge-symmetric_CAU-CAU | AACUUCGAAAGUGU | | ACTCCCTCG | | | | |
| | 0_1-1_mismatch_A-C | UCAUGCAUAGAUCC | | CGCAACTTC | | | | |
| | 6_1-1_mismatch_G-G | ACGGCUACAUAAUU | | GAAAGTGT | | | | |
| | 10_1-1_mismatch_U-C | CCUUUUGGAGUCAC | | TCATGCATA | | | | |
| | 17_3-1_bulge-asymmetric_AGG-G | UGUCGUCGAAUGGC | | GATCCACG | | | | |
| | 26_1-1_mismatch_A-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 65 | −18_6-6_internal_loop- | CUCAGCAGCAGCCA | 504 | CTCAGCAG | 0.464891 | 0.464891 | 0.463031 | 0.427536 |
| | symmetric_UGGUGU-UGGAGU | CAACUCCCUCGCGC | | CAGCCACA | | | | |
| | -6_3-3_bulge-symmetric_CAU-CAU | AACUUUGUUUGUCA | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GTTTGTCAT | | | | |
| | 10_1-1_mismatch_U-C | UCCUUUUGGAGUCA | | TCATGCATA | | | | |
| | 17_2-1_bulge-asymmetric_AG-A | CUGUCGUCGAAUGG | | GATCCACG | | | | |
| | 22_3-3_bulge-symmetric_UUU-UUU | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 66 | −18_6-6_internal_loop- | CUCAGCAGCAGCCA | 505 | CTCAGCAG | 0.467216 | 0.467216 | 0.463914 | 0.40943 |
| | symmetric_UGGUGU-UGGAGU | CAACUCCCUCGCGC | | CAGCCACA | | | | |
| | -6_3-3_bulge-symmetric_CAU-CAU | AACUUUGAAACUAU | | ACTCCCTCG | | | | |
| | 0_1-1_mismatch_A-C | CCUUUCAUGCAUAG | | CGCAACTTT | | | | |
| | 6_1-1_mismatch_G-G | AUCCACGGCUACAU | | GAAACTAT | | | | |
| | 10_1-1_mismatch_U-C | AAUUCCUUUUGGAG | | CCTTTCATG | | | | |
| | 21_1-3_bulge-asymmetric_C-CUA | UCACUGUCGUCGAA | | CATAGATCC | | | | |
| | 30_6-6_internal_loop- | UGGC | | ACGGCTAC | | | | |
| | symmetric_GCCAAG-GCGCAA | | | ATAATTCCT | | | | |
| | | | | TTTGGAGTC | | | | |
| | | | | ACTGTCGTC | | | | |
| | | | | GAATGGC | | | | |
| 67 | −18_6-6_internal_loop- | CUCAGCAGCAGCCA | 506 | CTCAGCAG | 0.478615 | 0.478615 | 0.467802 | 0.398507 |
| | symmetric_UGGUGU-UGGAGU | CAACUCCCUCGCGC | | CAGCCACA | | | | |
| | -6_3-3_bulge-symmetric_CAU-CAU | AACUUUGUCCAAGU | | ACTCCCTCG | | | | |
| | 0_1-1_mismatch_A-C | GCUUUCAUGCAUAG | | CGCAACTTT | | | | |
| | 6_1-1_mismatch_G-G | AUCCACGGCUACAU | | GTCCAAGT | | | | |
| | 10_1-1_mismatch_U-C | AAUUCCUUUUGGAG | | GCTTTCATG | | | | |
| | 19_1-1_mismatch_G-G | UCACUGUCGUCGAA | | CATAGATCC | | | | |
| | 24_1-3_bulge-asymmetric_U-UCC | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 68 | −18_6-6_internal_loop- | CUCAGCAGCAGCCA | 507 | CTCAGCAG | 0.466213 | 0.466213 | 0.455583 | 0.410947 |
| | symmetric_UGGUGU-UGGAGU | CAACUCCCUCGCGC | | CAGCCACA | | | | |
| | -6_3-3_bulge-symmetric_CAU-CAU | AACUUUGAACUCGU | | ACTCCCTCG | | | | |
| | 0_1-1_mismatch_A-C | CCUUUCAUGCAUAG | | CGCAACTTT | | | | |
| | 6_1-1_mismatch_G-G | AUCCACGGCUACAU | | GAACTCGTC | | | | |
| | 10_1-1_mismatch_U-C | AAUUCCUUUUGGAG | | CTTTCATGC | | | | |
| | 22_1-3_bulge-asymmetric_U-CUC | UCACUGUCGUCGAA | | ATAGATCC | | | | |
| | 30_6-6_internal_loop- | UGGC | | ACGGCTAC | | | | |
| | symmetric_GCCAAG-GCGCAA | | | ATAATTCCT | | | | |
| | | | | TTTGGAGTC | | | | |
| | | | | ACTGTCGTC | | | | |
| | | | | GAATGGC | | | | |
| 69 | −18_6-6_internal_loop- | CUCAGCAGCAGCCA | 508 | CTCAGCAG | 0.46212 | 0.46212 | 0.469336 | 0.409081 |
| | symmetric_UGGUGU-UGGAGU | CAACUCCCUCGCGC | | CAGCCACA | | | | |
| | -6_3-3_bulge-symmetric_CAU-CAU | AACUUAAAGAAAUA | | ACTCCCTCG | | | | |
| | 0_1-1_mismatch_A-C | CUCCUUUCAUGCAU | | CGCAACTTA | | | | |
| | 6_1-1_mismatch_G-G | AGAUCCACGGCUAC | | AAGAAATA | | | | |
| | 10_1-1_mismatch_U-C | AUAAUUCCUUUUGG | | CTCCTTTCA | | | | |
| | 21_1-3_bulge-asymmetric_C-UAC | AGUCACUGUCGUCG | | TGCATAGAT | | | | |
| | 26_1-3_bulge-asymmetric_A-AAA | AAUGGC | | CCACGGCT | | | | |
| | 30_6-6_internal_loop- | | | ACATAATTC | | | | |
| | symmetric_GCCAAG-GCGCAA | | | CTTTTGGAG | | | | |
| | | | | TCACTGTCG | | | | |
| | | | | TCGAATGG | | | | |
| | | | | C | | | | |
| 70 | −18_6-6_internal_loop- | CUCAGCAGCAGCCA | 509 | CTCAGCAG | 0.48054 | 0.48054 | 0.453638 | 0.420734 |
| | symmetric_UGGUGU-UGGAGU | CAACUCCCUCGCGC | | CAGCCACA | | | | |
| | -6_3-3_bulge-symmetric_CAU-CAU | AACUUGUAAAGUCC | | ACTCCCTCG | | | | |
| | 0_1-1_mismatch_A-C | ACAUGCAUAGAUCC | | CGCAACTTG | | | | |
| | 6_1-1_mismatch_G-G | ACGGCUACAUAAUU | | TAAAGTCC | | | | |
| | 10_1-1_mismatch_U-C | CCUUUUGGAGUCAC | | ACATGCAT | | | | |
| | 15_3-1_bulge-asymmetric_AAA-A | UGUCGUCGAAUGGC | | AGATCCAC | | | | |
| | 25_2-2_bulge-symmetric_CA-GU | | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 71 | −18_6-6_internal_loop- | CUCAGCAGCAGCCA | 510 | CTCAGCAG | 0.469288 | 0.469288 | 0.466662 | 0.419257 |
| | symmetric_UGGUGU-UGGAGU | CAACUCCCUCGCGC | | CAGCCACA | | | | |
| | -6_3-3_bulge-symmetric_CAU-CAU | AACUUUGAAAGGAG | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GAAAGGAG | | | | |
| | 10_1-1_mismatch_U-C | UUCCUUUUGGAGUC | | TTTCATGCA | | | | |
| | 18_3-3_bulge-symmetric_GGA-GAG | ACUGUCGUCGAAUG | | TAGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 72 | −18_6-6_internal_loop- | CUCAGCAGCAGCCA | 511 | CTCAGCAG | 0.490299 | 0.490299 | 0.467722 | 0.38757 |
| | symmetric_UGGUGU-UGGAGU | CAACUCCCUCGCGC | | CAGCCACA | | | | |
| | -6_3-3_bulge-symmetric_CAU-CAU | AACAUUGAAAGUCA | | ACTCCCTCG | | | | |
| | 0_1-1_mismatch_A-C | UUUCAUGCAUAGAU | | CGCAACATT | | | | |
| | 6_1-1_mismatch_G-G | CCACGGCUACAUAA | | GAAAGTCA | | | | |
| | 10_1-1_mismatch_U-C | UUCCUUUUGGAGUC | | TTTCATGCA | | | | |
| | 18_1-1_mismatch_G-A | ACUGUCGUCGAAUG | | TAGATCCAC | | | | |
| | 28_1-1_mismatch_A-A | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 73 | −18_6-6_internal_loop- | CUCAGCAGCAGCCA | 512 | CTCAGCAG | 0.486521 | 0.486521 | 0.451484 | 0.412735 |
| | symmetric_UGGUGU-UGGAGU | CAACUCCCUCGCGC | | CAGCCACA | | | | |
| | -6_3-3_bulge-symmetric_CAU-CAU | AACUUUGACUGCUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GACTGCTTT | | | | |
| | 10_1-1_mismatch_U-C | CCUUUUGGAGUCAC | | CATGCATA | | | | |
| | 19_1-1_mismatch_G-G | UGUCGUCGAAUGGC | | GATCCACG | | | | |
| | 21_3-1_bulge-asymmetric_CUU-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 74 | −18_6-6_internal_loop- | CUCAGCAGCAGCCA | 513 | CTCAGCAG | 0.50814 | 0.50814 | 0.449409 | 0.400094 |
| | symmetric_UGGUGU-UGGAGU | CAACUCCCUCGCGC | | CAGCCACA | | | | |
| | -6_3-3_bulge-symmetric_CAU-CAU | AACUUUGUGCCCUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GTGCCCTTT | | | | |
| | 10_1-1_mismatch_U-C | CCUUUUGGAGUCAC | | CATGCATA | | | | |
| | 20_1-1_mismatch_A-C | UGUCGUCGAAUGGC | | GATCCACG | | | | |
| | 22_3-1_bulge-asymmetric_UUU-U | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 75 | −18_6-6_internal_loop- | CUCAGCAGCAGCCA | 514 | CTCAGCAG | 0.457151 | 0.457151 | 0.483892 | 0.425975 |
| | symmetric_UGGUGU-UGGAGU | CAACUCCCUCGCGC | | CAGCCACA | | | | |
| | -6_3-3_bulge-symmetric_CAU-CAU | AACUUAAAAAGUGA | | ACTCCCTCG | | | | |
| | 0_1-1_mismatch_A-C | AUUCAUGCAUAGAU | | CGCAACTTA | | | | |
| | 6_1-1_mismatch_G-G | CCACGGCUACAUAA | | AAAAGTGA | | | | |
| | 10_1-1_mismatch_U-C | UUCCUUUUGGAGUC | | ATTCATGCA | | | | |
| | 17_3-3_bulge-symmetric_AGG-GAA | ACUGUCGUCGAAUG | | TAGATCCAC | | | | |
| | 25_2-2_bulge-symmetric_CA-AA | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 76 | −18_6-6_internal_loop- | CUCAGCAGCAGCCA | 515 | CTCAGCAG | 0.484274 | 0.484274 | 0.468672 | 0.390321 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCG | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GAAAGTCG | | | | |
| | 10_1-1_mismatch_U-C | UCCUUUUGGAGUCA | | TTCATGCAT | | | | |
| | 17_2-1_bulge-asymmetric_AG-G | CUGUCGUCGAAUGG | | AGATCCAC | | | | |
| | 30_6-6_internal_loop- | C | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 77 | −18_6-6_internal_loop- | CUCAGCAGCAGCCA | 516 | CTCAGCAG | 0.457319 | 0.457319 | 0.465007 | 0.407851 |
| | symmetric_UGGUGU-UGGAGU | CAACUCCCUCGCGC | | CAGCCACA | | | | |
| | -6_3-3_bulge-symmetric_CAU-CAU | AACGACGAAAGAUA | | ACTCCCTCG | | | | |
| | 0_1-1_mismatch_A-C | CCUUUCAUGCAUAG | | CGCAACGA | | | | |
| | 6_1-1_mismatch_G-G | AUCCACGGCUACAU | | CGAAAGAT | | | | |
| | 10_1-1_mismatch_U-C | AAUUCCUUUUGGAG | | ACCTTTCAT | | | | |
| | 20_1-3_bulge-asymmetric_A-AUA | UCACUGUCGUCGAA | | GCATAGAT | | | | |
| | 26_3-3_bulge-symmetric_AAA-GAC | UGGC | | CCACGGCT | | | | |
| | 30_6-6_internal_loop- | | | ACATAATTC | | | | |
| | symmetric_GCCAAG-GCGCAA | | | CTTTTGGAG | | | | |
| | | | | TCACTGTCG | | | | |
| | | | | TCGAATGG | | | | |
| | | | | C | | | | |
| 78 | −18_6-6_internal_loop- | CUCAGCAGCAGCCA | 517 | CTCAGCAG | 0.456259 | 0.456259 | 0.480623 | 0.416191 |
| | symmetric_UGGUGU-UGGAGU | CAACUCCCUCGCGC | | CAGCCACA | | | | |
| | -6_3-3_bulge-symmetric_CAU-CAU | AACUUAAAAGCGCC | | ACTCCCTCG | | | | |
| | 0_1-1_mismatch_A-C | UUUCAUGCAUAGAU | | CGCAACTTA | | | | |
| | 6_1-1_mismatch_G-G | CCACGGCUACAUAA | | AAAGCGCC | | | | |
| | 10_1-1_mismatch_U-C | UUCCUUUUGGAGUC | | TTTCATGCA | | | | |
| | 20_1-2_bulge-asymmetric_A-CG | ACUGUCGUCGAAUG | | TAGATCCAC | | | | |
| | 25_2-1_bulge-asymmetric_CA-A | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 79 | −18_6-6_internal_loop- | CUCAGCAGCAGCCA | 518 | CTCAGCAG | 0.473194 | 0.473194 | 0.457426 | 0.408751 |
| | symmetric_UGGUGU-UGGAGU | CAACUCCCUCGCGC | | CAGCCACA | | | | |
| | -6_3-3_bulge-symmetric_CAU-CAU | AACUUGUAAAGUCC | | ACTCCCTCG | | | | |
| | 0_1-1_mismatch_A-C | GAUUCAUGCAUAGA | | CGCAACTTG | | | | |
| | 6_1-1_mismatch_G-G | UCCACGGCUACAUA | | TAAAGTCC | | | | |
| | 10_1-1_mismatch_U-C | AUUCCUUUUGGAGU | | GATTCATGC | | | | |
| | 17_1-2_bulge-asymmetric_A-GA | CACUGUCGUCGAAU | | ATAGATCC | | | | |
| | 25_2-2_bulge-symmetric_CA-GU | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 80 | −18_6-6_internal_loop- | CUCAGCAGCAGCCA | 519 | CTCAGCAG | 0.459542 | 0.459542 | 0.470108 | 0.395769 |
| | symmetric_UGGUGU-UGGAGU | CAACUCCCUCGCGC | | CAGCCACA | | | | |
| | -6_3-3_bulge-symmetric_CAU-CAU | AACAUUGAAUUUCC | | ACTCCCTCG | | | | |
| | 0_1-1_mismatch_A-C | UUUCAUGCAUAGAU | | CGCAACATT | | | | |
| | 6_1-1_mismatch_G-G | CCACGGCUACAUAA | | GAATTTCCT | | | | |
| | 10_1-1_mismatch_U-C | UUCCUUUUGGAGUC | | TTCATGCAT | | | | |
| | 21_2-2_bulge-symmetric_CU-UU | ACUGUCGUCGAAUG | | AGATCCAC | | | | |
| | 28_1-1_mismatch_A-A | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 81 | −18_6-6_internal_loop- | CUCAGCAGCAGCCA | 520 | CTCAGCAG | 0.467687 | 0.467687 | 0.462637 | 0.409377 |
| | symmetric_UGGUGU-UGGAGU | CAACUCCCUCGCGC | | CAGCCACA | | | | |
| | -6_3-3_bulge-symmetric_CAU-CAU | AACUUUUCAAGUCA | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | TCAAGTCAT | | | | |
| | 10_1-1_mismatch_U-C | CCUUUUGGAGUCAC | | CATGCATA | | | | |
| | 16_3-1_bulge-asymmetric_AAG-A | UGUCGUCGAAUGGC | | GATCCACG | | | | |
| | 24_2-2_bulge-symmetric_UC-UC | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 82 | −18_6-6_internal_loop- | CUCAGCAGCAGCCA | 521 | CTCAGCAG | 0.463454 | 0.463454 | 0.452909 | 0.410053 |
| | symmetric_UGGUGU-UGGAGU | CAACUCCCUCGCGC | | CAGCCACA | | | | |
| | -6_3-3_bulge-symmetric_CAU-CAU | AACUCGGAAGGUCC | | ACTCCCTCG | | | | |
| | 0_1-1_mismatch_A-C | UUUCAUGCAUAGAU | | CGCAACTC | | | | |
| | 6_1-1_mismatch_G-G | CCACGGCUACAUAA | | GGAAGGTC | | | | |
| | 10_1-1_mismatch_U-C | UUCCUUUUGGAGUC | | CTTTCATGC | | | | |
| | 22_1-1_wobble_U-G | ACUGUCGUCGAAUG | | ATAGATCC | | | | |
| | 26_2-2_bulge-symmetric_AA-CG | GC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 83 | −18_6-6_internal_loop- | CUCAGCAGCAGCCA | 522 | CTCAGCAG | 0.463144 | 0.463144 | 0.468903 | 0.422551 |
| | symmetric_UGGUGU-UGGAGU | CAACUCCCUCGCGC | | CAGCCACA | | | | |
| | -6_3-3_bulge-symmetric_CAU-CAU | AACUUUGACUGUCC | | ACTCCCTCG | | | | |
| | 0_1-1_mismatch_A-C | GCGUUCAUGCAUAG | | CGCAACTTT | | | | |
| | 6_1-1_mismatch_G-G | AUCCACGGCUACAU | | GACTGTCCG | | | | |
| | 10_1-1_mismatch_U-C | AAUUCCUUUUGGAG | | CGTTCATGC | | | | |
| | 17_1-3_bulge-asymmetric_A-GCG | UCACUGUCGUCGAA | | ATAGATCC | | | | |
| | 22_2-2_bulge-symmetric_UU-CU | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 84 | −18_6-6_internal_loop- | CUCAGCAGCAGCCA | 523 | CTCAGCAG | 0.457467 | 0.457467 | 0.450896 | 0.417287 |
| | symmetric_UGGUGU-UGGAGU | CAACUCCCUCGCGC | | CAGCCACA | | | | |
| | -6_3-3_bulge-symmetric_CAU-CAU | AACUUUGAAAGUAU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GAAAGTAT | | | | |
| | 10_1-1_mismatch_U-C | UCCUUUUGGAGUCA | | TTCATGCAT | | | | |
| | 18_2-1_bulge-asymmetric_GG-A | CUGUCGUCGAAUGG | | AGATCCAC | | | | |
| | 30_6-6_internal_loop- | C | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 85 | −18_6-6_internal_loop- | CUCAGCAGCAGCCA | 524 | CTCAGCAG | 0.487412 | 0.487412 | 0.468986 | 0.415798 |
| | symmetric_UGGUGU-UGGAGU | CAACUCCCUCGCGC | | CAGCCACA | | | | |
| | -6_3-3_bulge-symmetric_CAU-CAU | AACUUUGAAAGAAC | | ACTCCCTCG | | | | |
| | 0_1-1_mismatch_A-C | UUAGCAUGCAUAGA | | CGCAACTTT | | | | |
| | 6_1-1_mismatch_G-G | UCCACGGCUACAUA | | GAAAGAAC | | | | |
| | 10_1-1_mismatch_U-C | AUUCCUUUUGGAGU | | TTAGCATGC | | | | |
| | 15_1-2_bulge-asymmetric_A-AG | CACUGUCGUCGAAU | | ATAGATCC | | | | |
| | 19_2-2_bulge-symmetric_GA-AA | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 86 | −18_6-6_internal_loop- | CUCAGCAGCAGCCA | 525 | CTCAGCAG | 0.453062 | 0.453062 | 0.477217 | 0.415565 |
| | symmetric_UGGUGU-UGGAGU | CAACUCCCUCGCGC | | CAGCCACA | | | | |
| | -6_3-3_bulge-symmetric_CAU-CAU | AACUUACAGAAAUA | | ACTCCCTCG | | | | |
| | 0_1-1_mismatch_A-C | ACUUUCAUGCAUAG | | CGCAACTTA | | | | |
| | 6_1-1_mismatch_G-G | AUCCACGGCUACAU | | CAGAAATA | | | | |
| | 10_1-1_mismatch_U-C | AAUUCCUUUUGGAG | | ACTTTCATG | | | | |
| | 19_3-3_bulge-symmetric_GAC-UAA | UCACUGUCGUCGAA | | CATAGATCC | | | | |
| | 26_1-3_bulge-asymmetric_A-ACA | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 87 | −18_6-6_internal_loop- | CUCAGCAGCAGCCA | 526 | CTCAGCAG | 0.468158 | 0.468158 | 0.458848 | 0.397717 |
| | symmetric_UGGUGU-UGGAGU | CAACUCCCUCGCGC | | CAGCCACA | | | | |
| | -6_3-3_bulge-symmetric_CAU-CAU | AACUCGGAUUCCUU | | ACTCCCTCG | | | | |
| | 0_1-1_mismatch_A-C | UCAUGCAUAGAUCC | | CGCAACTC | | | | |
| | 6_1-1_mismatch_G-G | ACGGCUACAUAAUU | | GGATTCCTT | | | | |
| | 10_1-1_mismatch_U-C | CCUUUUGGAGUCAC | | TCATGCATA | | | | |
| | 21_3-1_bulge-asymmetric_CUU-U | UGUCGUCGAAUGGC | | GATCCACG | | | | |
| | 26_2-2_bulge-symmetric_AA-CG | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 88 | −18_6-6_internal_loop- | CUCAGCAGCAGCCA | 527 | CTCAGCAG | 0.474524 | 0.474524 | 0.487895 | 0.404431 |
| | symmetric_UGGUGU-UGGAGU | CAACUCCCUCGCGC | | CAGCCACA | | | | |
| | -6_3-3_bulge-symmetric_CAU-CAU | AACUUUCUCAGUGC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | CTCAGTGCT | | | | |
| | 10_1-1_mismatch_U-C | UUCCUUUUGGAGUC | | TTCATGCAT | | | | |
| | 19_1-1_mismatch_G-G | ACUGUCGUCGAAUG | | AGATCCAC | | | | |
| | 23_3-3_bulge-symmetric_UUC-CUC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 89 | −18_6-6_internal_loop- | CUCAGCAGCAGCCA | 528 | CTCAGCAG | 0.449695 | 0.449695 | 0.478392 | 0.422659 |
| | symmetric_UGGUGU-UGGAGU | CAACUCCCUCGCGC | | CAGCCACA | | | | |
| | -6_3-3_bulge-symmetric_CAU-CAU | AACUUUUAGUCGUU | | ACTCCCTCG | | | | |
| | 0_1-1_mismatch_A-C | CAUGCAUAGAUCCA | | CGCAACTTT | | | | |
| | 6_1-1_mismatch_G-G | CGGCUACAUAAUUC | | TAGTCGTTC | | | | |
| | 10_1-1_mismatch_U-C | CUUUUGGAGUCACU | | ATGCATAG | | | | |
| | 17_2-1_bulge-asymmetric_AG-G | GUCGUCGAAUGGC | | ATCCACGG | | | | |
| | 23_3-1_bulge-asymmetric_UUC-U | | | CTACATAAT | | | | |
| | 30_6-6_internal_loop- | | | TCCTTTTGG | | | | |
| | symmetric_GCCAAG-GCGCAA | | | AGTCACTGT | | | | |
| | | | | CGTCGAAT | | | | |
| | | | | GGC | | | | |
| 90 | -6_6-6_internal_loop- | CUCAGCAGCAGCCA | 529 | CTCAGCAG | 0.451999 | 0.451999 | 0.466225 | 0.384298 |
| | symmetric_AUUCAU-UCAUAC | CAACUCCCUCGCGC | | CAGCCACA | | | | |
| | 0_1-1_mismatch_A-C | AACUGGAAAAGGAG | | ACTCCCTCG | | | | |
| | 6_3-3_bulge-symmetric_GUA-CCG | UUUCAUGAACCGAU | | CGCAACTG | | | | |
| | 18_3-3_bulge-symmetric_GGA-GAG | CCACGGCUAUCAUA | | GAAAAGGA | | | | |
| | 25_3-3_bulge-symmetric_CAA-GGA | CUCCUUUACACCAC | | GTTTCATGA | | | | |
| | 30_6-6_internal_loop- | ACUGUCGUCGAAUG | | ACCGATCC | | | | |
| | symmetric_GCCAAG-GCGCAA | GC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 91 | −18_6-6_internal_loop- | CUCAGCAGCAGCCA | 530 | CTCAGCAG | 0.450607 | 0.450607 | 0.470194 | 0.42613 |
| | symmetric_UGGUGU-UGGAGU | CAACUCCCUCGCGC | | CAGCCACA | | | | |
| | -6_3-3_bulge-symmetric_CAU-CAU | AACUUUGUAGUCGU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GTAGTCGTT | | | | |
| | 10_1-1_mismatch_U-C | CCUUUUGGAGUCAC | | CATGCATA | | | | |
| | 17_2-1_bulge-asymmetric_AG-G | UGUCGUCGAAUGGC | | GATCCACG | | | | |
| | 23_2-1_bulge-asymmetric_UU-U | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 92 | −18_6-6_internal_loop- | CUCAGCAGCAGCCA | 531 | CTCAGCAG | 0.459218 | 0.459218 | 0.43934 | 0.401455 |
| | symmetric_UGGUGU-UGGAGU | CAACUCCCUCGCGC | | CAGCCACA | | | | |
| | -6_3-3_bulge-symmetric_CAU-CAU | AACUUAGCUUGUCC | | ACTCCCTCG | | | | |
| | 0_1-1_mismatch_A-C | UUUCAUGCAUAGAU | | CGCAACTTA | | | | |
| | 6_1-1_mismatch_G-G | CCACGGCUACAUAA | | GCTTGTCCT | | | | |
| | 10_1-1_mismatch_U-C | UUCCUUUUGGAGUC | | TTCATGCAT | | | | |
| | 22_3-3_bulge-symmetric_UUU-CUU | ACUGUCGUCGAAUG | | AGATCCAC | | | | |
| | 26_1-1_mismatch_A-A | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 93 | −18_6-6_internal_loop- | CUCAGCAGCAGCCA | 532 | CTCAGCAG | 0.47793 | 0.47793 | 0.479465 | 0.392419 |
| | symmetric_UGGUGU-UGGAGU | CAACUCCCUCGCGC | | CAGCCACA | | | | |
| | -6_3-3_bulge-symmetric_CAU-CAU | AACUUUGACCAGUC | | ACTCCCTCG | | | | |
| | 0_1-1_mismatch_A-C | CUUGCAUGCAUAGA | | CGCAACTTT | | | | |
| | 6_1-1_mismatch_G-G | UCCACGGCUACAUA | | GACCAGTC | | | | |
| | 10_1-1_mismatch_U-C | AUUCCUUUUGGAGU | | CTTGCATGC | | | | |
| | 15_1-1_mismatch_A-G | CACUGUCGUCGAAU | | ATAGATCC | | | | |
| | 23_1-2_bulge-asymmetric_U-CC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 94 | −18_6-6_internal_loop- | CUCAGCAGCAGCCA | 533 | CTCAGCAG | 0.460113 | 0.460113 | 0.44623 | 0.402222 |
| | symmetric_UGGUGU-UGGAGU | CAACUCCCUCGCGC | | CAGCCACA | | | | |
| | -6_3-3_bulge-symmetric_CAU-CAU | AACUCAUGAAAUUA | | ACTCCCTCG | | | | |
| | 0_1-1_mismatch_A-C | CUUUCAUGCAUAGA | | CGCAACTC | | | | |
| | 6_1-1_mismatch_G-G | UCCACGGCUACAUA | | ATGAAATT | | | | |
| | 10_1-1_mismatch_U-C | AUUCCUUUUGGAGU | | ACTTTCATG | | | | |
| | 19_3-3_bulge-symmetric_GAC-UUA | CACUGUCGUCGAAU | | CATAGATCC | | | | |
| | 27_1-2_bulge-asymmetric_A-CA | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 95 | −18_6-6_internal_loop- | CUCAGCAGCAGCCA | 534 | CTCAGCAG | 0.457225 | 0.457225 | 0.484284 | 0.396422 |
| | symmetric_UGGUGU-UGGAGU | CAACUCCCUCGCGC | | CAGCCACA | | | | |
| | -6_3-3_bulge-symmetric_CAU-CAU | AACUUCGAAAGGGC | | ACTCCCTCG | | | | |
| | 0_1-1_mismatch_A-C | UUUCAUGCAUAGAU | | CGCAACTTC | | | | |
| | 6_1-1_mismatch_G-G | CCACGGCUACAUAA | | GAAAGGGC | | | | |
| | 10_1-1_mismatch_U-C | UUCCUUUUGGAGUC | | TTTCATGCA | | | | |
| | 19_2-2_bulge-symmetric_GA-GG | ACUGUCGUCGAAUG | | TAGATCCAC | | | | |
| | 26_1-1_mismatch_A-C | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 96 | −18_6-6_internal_loop- | CUCAGCAGCAGCCA | 535 | CTCAGCAG | 0.454876 | 0.454876 | 0.47166 | 0.399386 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCA | | ACTCCCTCG | | | | |
| | 0_1-1_mismatch_A-C | GUUCAUGCAUAGAU | | CGCAACTTT | | | | |
| | 6_1-1_mismatch_G-G | CCACGGCUACAUAA | | GAAAGTCA | | | | |
| | 10_1-1_mismatch_U-C | UUCCUUUUGGAGUC | | GTTCATGCA | | | | |
| | 17_2-2_bulge-symmetric_AG-AG | ACUGUCGUCGAAUG | | TAGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 97 | −18_6-6_internal_loop- | CUCAGCAGCAGCCA | 536 | CTCAGCAG | 0.456766 | 0.456766 | 0.45821 | 0.397874 |
| | symmetric_UGGUGU-UGGAGU | CAACUCCCUCGCGC | | CAGCCACA | | | | |
| | -6_3-3_bulge-symmetric_CAU-CAU | AACUUUGAAUUUAA | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GAATTTAAT | | | | |
| | 10_1-1_mismatch_U-C | UUCCUUUUGGAGUC | | TTCATGCAT | | | | |
| | 18_2-2_bulge-symmetric_GG-AA | ACUGUCGUCGAAUG | | AGATCCAC | | | | |
| | 21_2-2_bulge-symmetric_CU-UU | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 98 | −18_6-6_internal_loop- | CUCAGCAGCAGCCA | 537 | CTCAGCAG | 0.438263 | 0.438263 | 0.471657 | 0.403787 |
| | symmetric_UGGUGU-UGGAGU | CAACUCCCUCGCGC | | CAGCCACA | | | | |
| | -6_3-3_bulge-symmetric_CAU-CAU | AACUUAAAAAACUU | | ACTCCCTCG | | | | |
| | 0_1-1_mismatch_A-C | CCUUUCAUGCAUAG | | CGCAACTTA | | | | |
| | 6_1-1_mismatch_G-G | AUCCACGGCUACAU | | AAAAACTT | | | | |
| | 10_1-1_mismatch_U-C | AAUUCCUUUUGGAG | | CCTTTCATG | | | | |
| | 21_1-3_bulge-asymmetric_C-ACU | UCACUGUCGUCGAA | | CATAGATCC | | | | |
| | 25_2-2_bulge-symmetric_CA-AA | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 99 | -6_6-6_internal_loop- | CUCAGCAGCAGCCA | 538 | CTCAGCAG | 0.446052 | 0.446052 | 0.44916 | 0.37149 |
| | symmetric_AUUCAU-UCAUAC | CAACUCCCUCGCGC | | CAGCCACA | | | | |
| | 0_1-1_mismatch_A-C | AACUUUCGUAGAUU | | ACTCCCTCG | | | | |
| | 8_3-3_bulge-symmetric_AUU-UCG | CAUGUCGACAUCCA | | CGCAACTTT | | | | |
| | 17_3-3_bulge-symmetric_AGG-AGA | CGGCUAUCAUACUC | | CGTAGATTC | | | | |
| | 22_4-1_bulge-asymmetric_UUUC-C | CUUUACACCACACU | | ATGTCGAC | | | | |
| | 30_6-6_internal_loop- | GUCGUCGAAUGGC | | ATCCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 100 | −18_6-6_internal_loop- | CUCAGCAGCAGCCA | 539 | CTCAGCAG | 0.467253 | 0.467253 | 0.439591 | 0.433892 |
| | symmetric_UGGUGU-UGGAGU | CAACUCCCUCGCGC | | CAGCCACA | | | | |
| | -6_3-3_bulge-symmetric_CAU-CAU | AACUUUGUGUAUUU | | ACTCCCTCG | | | | |
| | 0_1-1_mismatch_A-C | CAUGCAUAGAUCCA | | CGCAACTTT | | | | |
| | 6_1-1_mismatch_G-G | CGGCUACAUAAUUC | | GTGTATTTC | | | | |
| | 10_1-1_mismatch_U-C | CUUUUGGAGUCACU | | ATGCATAG | | | | |
| | 18_2-1_bulge-asymmetric_GG-A | GUCGUCGAAUGGC | | ATCCACGG | | | | |
| | 22_3-1_bulge-asymmetric_UUU-U | | | CTACATAAT | | | | |
| | 30_6-6_internal_loop- | | | TCCTTTTGG | | | | |
| | symmetric_GCCAAG-GCGCAA | | | AGTCACTGT | | | | |
| | | | | CGTCGAAT | | | | |
| | | | | GGC | | | | |
| 101 | −18_6-6_internal_loop- | CUCAGCAGCAGCCA | 540 | CTCAGCAG | 0.462814 | 0.462814 | 0.447535 | 0.395798 |
| | symmetric_UGGUGU-UGGAGU | CAACUCCCUCGCGC | | CAGCCACA | | | | |
| | -6_3-3_bulge-symmetric_CAU-CAU | AACUUCAAAGUCCU | | ACTCCCTCG | | | | |
| | 0_1-1_mismatch_A-C | UUCAUGCAUAGAUC | | CGCAACTTC | | | | |
| | 6_1-1_mismatch_G-G | CACGGCUACAUAAU | | AAAGTCCTT | | | | |
| | 10_1-1_mismatch_U-C | UCCUUUUGGAGUCA | | TCATGCATA | | | | |
| | 25_2-1_bulge-asymmetric_CA-C | CUGUCGUCGAAUGG | | GATCCACG | | | | |
| | 30_6-6_internal_loop- | C | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 102 | −18_6-6_internal_loop- | CUCAGCAGCAGCCA | 541 | CTCAGCAG | 0.456969 | 0.456969 | 0.465385 | 0.424785 |
| | symmetric_UGGUGU-UGGAGU | CAACUCCCUCGCGC | | CAGCCACA | | | | |
| | -6_3-3_bulge-symmetric_CAU-CAU | AACUUUGAAACUAA | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GAAACTAA | | | | |
| | 10_1-1_mismatch_U-C | UUCCUUUUGGAGUC | | TTTCATGCA | | | | |
| | 18_2-2_bulge-symmetric_GG-AA | ACUGUCGUCGAAUG | | TAGATCCAC | | | | |
| | 21_1-1_mismatch_C-C | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 103 | −18_6-6_internal_loop- | CUCAGCAGCAGCCA | 542 | CTCAGCAG | 0.451065 | 0.451065 | 0.445188 | 0.419741 |
| | symmetric_UGGUGU-UGGAGU | CAACUCCCUCGCGC | | CAGCCACA | | | | |
| | -6_3-3_bulge-symmetric_CAU-CAU | AACUUUCAGAUAUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | CAGATATTT | | | | |
| | 10_1-1_mismatch_U-C | CCUUUUGGAGUCAC | | CATGCATA | | | | |
| | 18_3-3_bulge-symmetric_GGA-AUA | UGUCGUCGAAUGGC | | GATCCACG | | | | |
| | 23_3-1_bulge-asymmetric_UUC-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 104 | −18_6-6_internal_loop- | CUCAGCAGCAGCCA | 543 | CTCAGCAG | 0.465105 | 0.465105 | 0.455022 | 0.385578 |
| | symmetric_UGGUGU-UGGAGU | CAACUCCCUCGCGC | | CAGCCACA | | | | |
| | -6_3-3_bulge-symmetric_CAU-CAU | AACUAUGAAGGUCC | | ACTCCCTCG | | | | |
| | 0_1-1_mismatch_A-C | UUUCAUGCAUAGAU | | CGCAACTAT | | | | |
| | 6_1-1_mismatch_G-G | CCACGGCUACAUAA | | GAAGGTCC | | | | |
| | 10_1-1_mismatch_U-C | UUCCUUUUGGAGUC | | TTTCATGCA | | | | |
| | 22_1-1_wobble_U-G | ACUGUCGUCGAAUG | | TAGATCCAC | | | | |
| | 27_1-1_mismatch_A-A | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 105 | −18_6-6_internal_loop- | CUCAGCAGCAGCCA | 544 | CTCAGCAG | 0.42404 | 0.42404 | 0.463635 | 0.401729 |
| | symmetric_UGGUGU-UGGAGU | CAACUCCCUCGCGC | | CAGCCACA | | | | |
| | −6_3-3_bulge-symmetric_CAU-CAU | AACGAAGAACUGUC | | ACTCCCTCG | | | | |
| | 0_1-1_mismatch_A-C | CUUUCAUGCAUAGA | | CGCAACGA | | | | |
| | 6_1-1_mismatch_G-G | UCCACGGCUACAUA | | AGAACTGT | | | | |
| | 10_1-1_mismatch_U-C | AUUCCUUUUGGAGU | | CCTTTCATG | | | | |
| | 22_1-2_bulge-asymmetric_U-CU | CACUGUCGUCGAAU | | CATAGATCC | | | | |
| | 26_3-3_bulge-symmetric_AAA-GAA | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 106 | −18_6−6_internal_loop- | CUCAGCAGCAGCCA | 545 | CTCAGCAG | 0.462136 | 0.462136 | 0.457684 | 0.390279 |
| | symmetric_UGGUGU-UGGAGU | CAACUCCCUCGCGC | | CAGCCACA | | | | |
| | −6_3-3_bulge-symmetric_CAU-CAU | AACUUUGAAAGUGC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GAAAGTGC | | | | |
| | 10_1-1_mismatch_U-C | UUCCUUUUGGAGUC | | TTTCATGCA | | | | |
| | 19_1-1_mismatch_G-G | ACUGUCGUCGAAUG | | TAGATCCAC | | | | |
| | 30_6−6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 107 | −6_6−6_internal_loop- | CUCAGCAGCAGCCA | 546 | CTCAGCAG | 0.438003 | 0.438003 | 0.449196 | 0.386623 |
| | symmetric_AUUCAU-UCAUAC | CAACUCCCUCGCGC | | CAGCCACA | | | | |
| | 0_1-1_mismatch_A-C | AACUUGCUAAGGAG | | ACTCCCTCG | | | | |
| | 7_3-4_bulge-asymmetric_UAU-UAGU | UUUCAUGAUAGUCA | | CGCAACTTG | | | | |
| | 18_3-3_bulge-symmetric_GGA-GAG | UCCACGGCUAUCAU | | CTAAGGAG | | | | |
| | 24_3-3_bulge-symmetric_UCA-GCU | ACUCCUUUACACCA | | TTTCATGAT | | | | |
| | 30_6−6_internal_loop- | CACUGUCGUCGAAU | | AGTCATCCA | | | | |
| | symmetric_GCCAAG-GCGCAA | GGC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 108 | −6_6−6_internal_loop- | CUCAGCAGCAGCCA | 547 | CTCAGCAG | 0.418572 | 0.418572 | 0.486851 | 0.382145 |
| | symmetric_AUUCAU-UCAUAC | CAACUCCCUCGCGC | | CAGCCACA | | | | |
| | 0_1-1_mismatch_A-C | AACGGAGAAAGGGA | | ACTCCCTCG | | | | |
| | 7_3-3_bulge-symmetric_UAU-CAC | UUUCAUGACACCAU | | CGCAACGG | | | | |
| | 18_3-3_bulge-symmetric_GGA-GGA | CCACGGCUAUCAUA | | AGAAAGGG | | | | |
| | 26_3-3_bulge-symmetric_AAA-GGA | CUCCUUUACACCAC | | ATTTCATGA | | | | |
| | 30_6−6_internal_loop- | ACUGUCGUCGAAUG | | CACCATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 109 | −18_6−6_internal_loop- | CUCAGCAGCAGCCA | 548 | CTCAGCAG | 0.476267 | 0.476267 | 0.4493 | 0.413246 |
| | symmetric_UGGUGU-UGGAGU | CAACUCCCUCGCGC | | CAGCCACA | | | | |
| | −6_3-3_bulge-symmetric_CAU-CAU | AACUUUGAUCAGUC | | ACTCCCTCG | | | | |
| | 0_1-1_mismatch_A-C | CGGGCAUGCAUAGA | | CGCAACTTT | | | | |
| | 6_1-1_mismatch_G-G | UCCACGGCUACAUA | | GATCAGTCC | | | | |
| | 10_1-1_mismatch_U-C | AUUCCUUUUGGAGU | | GGGCATGC | | | | |
| | 15_3-3_bulge-symmetric_AAA-GGG | CACUGUCGUCGAAU | | ATAGATCC | | | | |
| | 23_1-2_bulge-asymmetric_U-UC | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 110 | −18_6−6_internal_loop- | CUCAGCAGCAGCCA | 549 | CTCAGCAG | 0.457941 | 0.457941 | 0.482419 | 0.395252 |
| | symmetric_UGGUGU-UGGAGU | CAACUCCCUCGCGC | | CAGCCACA | | | | |
| | −6_3-3_bulge-symmetric_CAU-CAU | AACUUUGCAAGUCC | | ACTCCCTCG | | | | |
| | 0_1-1_mismatch_A-C | CAUUCAUGCAUAGA | | CGCAACTTT | | | | |
| | 6_1-1_mismatch_G-G | UCCACGGCUACAUA | | GCAAGTCC | | | | |
| | 10_1-1_mismatch_U-C | AUUCCUUUUGGAGU | | CATTCATGC | | | | |
| | 17_1-2_bulge-asymmetric_A-CA | CACUGUCGUCGAAU | | ATAGATCC | | | | |
| | 24_1-1_mismatch_U-C | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 111 | −18_6−6_internal_loop- | CUCAGCAGCAGCCA | 550 | CTCAGCAG | 0.434081 | 0.434081 | 0.470325 | 0.40865 |
| | symmetric_UGGUGU-UGGAGU | CAACUCCCUCGCGC | | CAGCCACA | | | | |
| | −6_3-3_bulge-symmetric_CAU-CAU | AACUUUCUCAAAGU | | ACTCCCTCG | | | | |
| | 0_1-1_mismatch_A-C | CCUUUCAUGCAUAG | | CGCAACTTT | | | | |
| | 6_1-1_mismatch_G-G | AUCCACGGCUACAU | | CTCAAAGTC | | | | |
| | 10_1-1_mismatch_U-C | AAUUCCUUUUGGAG | | CTTTCATGC | | | | |
| | 25_1-3_bulge-asymmetric_C-CUC | UCACUGUCGUCGAA | | ATAGATCC | | | | |
| | 30_6−6_internal_loop- | UGGC | | ACGGCTAC | | | | |
| | symmetric_GCCAAG-GCGCAA | | | ATAATTCCT | | | | |
| | | | | TTTGGAGTC | | | | |
| | | | | ACTGTCGTC | | | | |
| | | | | GAATGGC | | | | |
| 112 | −18_6−6_internal_loop- | CUCAGCAGCAGCCA | 551 | CTCAGCAG | 0.465136 | 0.465136 | 0.45359 | 0.388317 |
| | symmetric_UGGUGU-UGGAGU | CAACUCCCUCGCGC | | CAGCCACA | | | | |
| | −6_3-3_bulge-symmetric_CAU-CAU | AACUGUGAAAAUCC | | ACTCCCTCG | | | | |
| | 0_1-1_mismatch_A-C | UUUCAUGCAUAGAU | | CGCAACTGT | | | | |
| | 6_1-1_mismatch_G-G | CCACGGCUACAUAA | | GAAAATCC | | | | |
| | 10_1-1_mismatch_U-C | UUCCUUUUGGAGUC | | TTTCATGCA | | | | |
| | 21_1-1_mismatch_C-A | ACUGUCGUCGAAUG | | TAGATCCAC | | | | |
| | 27_1-1_mismatch_A-G | GC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 113 | −18_6−6_internal_loop- | CUCAGCAGCAGCCA | 552 | CTCAGCAG | 0.426532 | 0.426532 | 0.454278 | 0.400828 |
| | symmetric_UGGUGU-UGGAGU | CAACUCCCUCGCGC | | CAGCCACA | | | | |
| | −6_3-3_bulge-symmetric_CAU-CAU | AACUUUUUAAGUCA | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | TTAAGTCAT | | | | |
| | 10_1-1_mismatch_U-C | UCCUUUUGGAGUCA | | TCATGCATA | | | | |
| | 17_2-1_bulge-asymmetric_AG-A | CUGUCGUCGAAUGG | | GATCCACG | | | | |
| | 24_2-2_bulge-symmetric_UC-UU | C | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 114 | −18_6−6_internal_loop- | CUCAGCAGCAGCCA | 553 | CTCAGCAG | 0.451268 | 0.451268 | 0.434655 | 0.396351 |
| | symmetric_UGGUGU-UGGAGU | CAACUCCCUCGCGC | | CAGCCACA | | | | |
| | −6_3-3_bulge-symmetric_CAU-CAU | AACUUUGAAAGAUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GAAAGATT | | | | |
| | 10_1-1_mismatch_U-C | CCUUUUGGAGUCAC | | TCATGCATA | | | | |
| | 18_3-1_bulge-asymmetric_GGA-A | UGUCGUCGAAUGGC | | GATCCACG | | | | |
| | 30_6−6_internal_loop- | | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 115 | −18_6−6_internal_loop- | CUCAGCAGCAGCCA | 554 | CTCAGCAG | 0.415824 | 0.415824 | 0.487147 | 0.410267 |
| | symmetric_UGGUGU-UGGAGU | CAACUCCCUCGCGC | | CAGCCACA | | | | |
| | −6_3-3_bulge-symmetric_CAU-CAU | AACUUUGAAAGUCC | | ACTCCCTCG | | | | |
| | 0_1-1_mismatch_A-C | UGGCAUGCAUAGAC | | CGCAACTTT | | | | |
| | 4_1-0_bulge-asymmetric_A- | CACGGCUACAUAAU | | GAAAGTCC | | | | |
| | 6_1-1_mismatch_G-G | UCCUUUUGGAGUCA | | TGGCATGC | | | | |
| | 10_1-1_mismatch_U-C | CUGUCGUCGAAUGG | | ATAGACCA | | | | |
| | 15_2-2_bulge-symmetric_AA-GG | C | | CGGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 116 | −6_6−6_internal_loop- | CUCAGCAGCAGCCA | 555 | CTCAGCAG | 0.424496 | 0.424496 | 0.465151 | 0.405997 |
| | symmetric_AUUCAU-UCAUAC | CAACUCCCUCGCGC | | CAGCCACA | | | | |
| | 0_1-1_mismatch_A-C | AACUUUGUUUGGGG | | ACTCCCTCG | | | | |
| | 7_3-3_bulge-symmetric_UAU-UGU | UUUCAUGAUGUCAU | | CGCAACTTT | | | | |
| | 18_3-3_bulge-symmetric_GGA-GGG | CCACGGCUAUCAUA | | GTTTGGGGT | | | | |
| | 22_3-3_bulge-symmetric_UUU-UUU | CUCCUUUACACCAC | | TTCATGATG | | | | |
| | 30_6−6_internal_loop- | ACUGUCGUCGAAUG | | TCATCCACG | | | | |
| | symmetric_GCCAAG-GCGCAA | GC | | GCTATCATA | | | | |
| | | | | CTCCTTTAC | | | | |
| | | | | ACCACACT | | | | |
| | | | | GTCGTCGA | | | | |
| | | | | ATGGC | | | | |
| 117 | −18_6−6_internal_loop- | CUCAGCAGCAGCCA | 556 | CTCAGCAG | 0.437859 | 0.437859 | 0.454702 | 0.40612 |
| | symmetric_UGGUGU-UGGAGU | CAACUCCCUCGCGC | | CAGCCACA | | | | |
| | −6_3-3_bulge-symmetric_CAU-CAU | AACUUUCCAAAGUC | | ACTCCCTCG | | | | |
| | 0_1-1_mismatch_A-C | AUCAUGCAUAGAUC | | CGCAACTTT | | | | |
| | 6_1-1_mismatch_G-G | CACGGCUACAUAAU | | CCAAAGTC | | | | |
| | 10_1-1_mismatch_U-C | UCCUUUUGGAGUCA | | ATCATGCAT | | | | |
| | 16_3-1_bulge-asymmetric_AAG-A | CUGUCGUCGAAUGG | | AGATCCAC | | | | |
| | 25_1-2_bulge-asymmetric_C-CC | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 118 | −18_6−6_internal_loop- | CUCAGCAGCAGCCA | 557 | CTCAGCAG | 0.502159 | 0.502159 | 0.441092 | 0.433188 |
| | symmetric_UGGUGU-UGGAGU | CAACUCCCUCGCGC | | CAGCCACA | | | | |
| | −6_3-3_bulge-symmetric_CAU-CAU | AACUUUGAUUCAUU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GATTCATTT | | | | |
| | 10_1-1_mismatch_U-C | CCUUUUGGAGUCAC | | CATGCATA | | | | |
| | 18_1-1_mismatch_G-A | UGUCGUCGAAUGGC | | GATCCACG | | | | |
| | 21_3-1_bulge-asymmetric_CUU-U | | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 119 | −18_6−6_internal_loop- | CUCAGCAGCAGCCA | 558 | CTCAGCAG | 0.424271 | 0.424271 | 0.455023 | 0.397111 |
| | symmetric_UGGUGU-UGGAGU | CAACUCCCUCGCGC | | CAGCCACA | | | | |
| | −6_3-3_bulge-symmetric_CAU-CAU | AACUUGUCGAAAGU | | ACTCCCTCG | | | | |
| | 0_1-1_mismatch_A-C | CCUUUCAUGCAUAG | | CGCAACTTG | | | | |
| | 3_1-2_bulge-asymmetric_G-GG | AUGGCACGGCUACA | | TCGAAAGT | | | | |
| | 6_1-1_mismatch_G-G | UAAUUCCUUUUGGA | | CCTTTCATG | | | | |
| | 10_1-1_mismatch_U-C | GUCACUGUCGUCGA | | CATAGATG | | | | |
| | 26_1-3_bulge-asymmetric_A-GUC | AUGGC | | GCACGGCT | | | | |
| | 30_6−6_internal_loop- | | | ACATAATTC | | | | |
| | symmetric_GCCAAG-GCGCAA | | | CTTTTGGAG | | | | |
| | | | | TCACTGTCG | | | | |
| | | | | TCGAATGG | | | | |
| | | | | C | | | | |
| 120 | −6_6−6_internal_loop- | CUCAGCAGCAGCCA | 559 | CTCAGCAG | 0.455501 | 0.455501 | 0.436346 | 0.380184 |
| | symmetric_AUUCAU-UCAUAC | CAACUCCCUCGCGC | | CAGCCACA | | | | |
| | 0_1-1_mismatch_A-C | AACUAGCUAAAGGA | | ACTCCCTCG | | | | |
| | 4_3-3_bulge-symmetric_AUG-GCC | GUUUCAUGAAUAGC | | CGCAACTA | | | | |
| | 18_3-3_bulge-symmetric_GGA-GAG | CCCACGGCUAUCAU | | GCTAAAGG | | | | |
| | 25_3-4_bulge-asymmetric_CAA-AGCU | ACUCCUUUACACCA | | AGTTTCATG | | | | |
| | 30_6−6_internal_loop- | CACUGUCGUCGAAU | | AATAGCCC | | | | |
| | symmetric_GCCAAG-GCGCAA | GGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 121 | −18_6−6_internal_loop- | CUCAGCAGCAGCCA | 560 | CTCAGCAG | 0.422394 | 0.422394 | 0.438586 | 0.399686 |
| | symmetric_UGGUGU-UGGAGU | CAACUCCCUCGCGC | | CAGCCACA | | | | |
| | −6_3-3_bulge-symmetric_CAU-CAU | AACGCUGAAAGUCC | | ACTCCCTCG | | | | |
| | 0_1-1_mismatch_A-C | UUUCAUGCAUAGAU | | CGCAACGC | | | | |
| | 6_1-1_mismatch_G-G | CCACGGCUACAUAA | | TGAAAGTC | | | | |
| | 10_1-1_mismatch_U-C | UUCCUUUUGGAGUC | | CTTTCATGC | | | | |
| | 27_2-2_bulge-symmetric_AA-GC | ACUGUCGUCGAAUG | | ATAGATCC | | | | |
| | 30_6−6_internal_loop- | GC | | ACGGCTAC | | | | |
| | symmetric_GCCAAG-GCGCAA | | | ATAATTCCT | | | | |
| | | | | TTTGGAGTC | | | | |
| | | | | ACTGTCGTC | | | | |
| | | | | GAATGGC | | | | |
| 122 | −6_6−6_internal_loop- | CUCAGCAGCAGCCA | 561 | CTCAGCAG | 0.408732 | 0.408732 | 0.49185 | 0.39111 |
| | symmetric_AUUCAU-UCAUAC | CAACUCCCUCGCGC | | CAGCCACA | | | | |
| | 0_1-1_mismatch_A-C | AACUGCCAAAGGAG | | ACTCCCTCG | | | | |
| | 7_4-4_bulge-symmetric_UAUU-UACC | UUUCAUGUACCCAU | | CGCAACTG | | | | |
| | 18_3-3_bulge-symmetric_GGA-GAG | CCACGGCUAUCAUA | | CCAAAGGA | | | | |
| | 25_3-3_bulge-symmetric_CAA-GCC | CUCCUUUACACCAC | | GTTTCATGT | | | | |
| | 30_6−6_internal_loop- | ACUGUCGUCGAAUG | | ACCCATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 123 | −18_6−6_internal_loop- | CUCAGCAGCAGCCA | 562 | CTCAGCAG | 0.45789 | 0.45789 | 0.462786 | 0.396407 |
| | symmetric_UGGUGU-UGGAGU | CAACUCCCUCGCGC | | CAGCCACA | | | | |
| | −6_3-3_bulge-symmetric_CAU-CAU | AACUUCAAGUCCUU | | ACTCCCTCG | | | | |
| | 0_1-1_mismatch_A-C | UCAUGCAUAGAUCC | | CGCAACTTC | | | | |
| | 6_1-1_mismatch_G-G | ACGGCUACAUAAUU | | AAGTCCTTT | | | | |
| | 10_1-1_mismatch_U-C | CCUUUUGGAGUCAC | | CATGCATA | | | | |
| | 24_3-1_bulge-asymmetric_UCA-C | UGUCGUCGAAUGGC | | GATCCACG | | | | |
| | 30_6−6_internal_loop- | | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 124 | −18_6−6_internal_loop- | CUCAGCAGCAGCCA | 563 | CTCAGCAG | 0.479049 | 0.479049 | 0.451861 | 0.423384 |
| | symmetric_UGGUGU-UGGAGU | CAACUCCCUCGCGC | | CAGCCACA | | | | |
| | −6_3-3_bulge-symmetric_CAU-CAU | AACUUUGAAAGACA | | ACTCCCTCG | | | | |
| | 0_1-1_mismatch_A-C | CAUCAUGCAUAGAU | | CGCAACTTT | | | | |
| | 6_1-1_mismatch_G-G | CCACGGCUACAUAA | | GAAAGACA | | | | |
| | 10_1-1_mismatch_U-C | UUCCUUUUGGAGUC | | CATCATGCA | | | | |
| | 16_3-3_bulge-symmetric_AAG-ACA | ACUGUCGUCGAAUG | | TAGATCCAC | | | | |
| | 20_1-1_mismatch_A-A | GC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 125 | −6_6−6_internal_loop- | CUCAGCAGCAGCCA | 564 | CTCAGCAG | 0.417847 | 0.417847 | 0.4498 | 0.377967 |
| | symmetric_AUUCAU-UCAUAC | CAACUCCCUCGCGC | | CAGCCACA | | | | |
| | 0_1-1_mismatch_A-C | AACUACAUAAGGAG | | ACTCCCTCG | | | | |
| | 9_3-3_bulge-symmetric_UUC-CGU | UUUCAUCGUUACAU | | CGCAACTA | | | | |
| | 18_3-3_bulge-symmetric_GGA-GAG | CCACGGCUAUCAUA | | CATAAGGA | | | | |
| | 24_4-4_bulge-symmetric_UCAA-ACAU | CUCCUUUACACCAC | | GTTTCATCG | | | | |
| | 30_6−6_internal_loop- | ACUGUCGUCGAAUG | | TTACATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 126 | −18_6−6_internal_loop- | CUCAGCAGCAGCCA | 565 | CTCAGCAG | 0.458666 | 0.458666 | 0.459228 | 0.410161 |
| | symmetric_UGGUGU-UGGAGU | CAACUCCCUCGCGC | | CAGCCACA | | | | |
| | −6_3-3_bulge-symmetric_CAU-CAU | AACUUUGAAAUAAU | | ACTCCCTCG | | | | |
| | 0_1-1_mismatch_A-C | CACUUCAUGCAUAG | | CGCAACTTT | | | | |
| | 6_1-1_mismatch_G-G | AUCCACGGCUACAU | | GAAATAAT | | | | |
| | 10_1-1_mismatch_U-C | AAUUCCUUUUGGAG | | CACTTCATG | | | | |
| | 17_2-2_bulge-symmetric_AG-AC | UCACUGUCGUCGAA | | CATAGATCC | | | | |
| | 21_1-3_bulge-asymmetric_C-UAA | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 127 | −18_6−6_internal_loop- | CUCAGCAGCAGCCA | 566 | CTCAGCAG | 0.472416 | 0.472416 | 0.444973 | 0.418004 |
| | symmetric_UGGUGU-UGGAGU | CAACUCCCUCGCGC | | CAGCCACA | | | | |
| | −6_3-3_bulge-symmetric_CAU-CAU | AACUUUGAAAGCAC | | ACTCCCTCG | | | | |
| | 0_1-1_mismatch_A-C | CAAUCAUGCAUAGA | | CGCAACTTT | | | | |
| | 6_1-1_mismatch_G-G | UCCACGGCUACAUA | | GAAAGCAC | | | | |
| | 10_1-1_mismatch_U-C | AUUCCUUUUGGAGU | | CAATCATGC | | | | |
| | 16_2-2_bulge-symmetric_AA-AA | CACUGUCGUCGAAU | | ATAGATCC | | | | |
| | 20_1-2_bulge-asymmetric_A-CA | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 128 | −18_6−6_internal_loop- | CUCAGCAGCAGCCA | 567 | CTCAGCAG | 0.453086 | 0.453086 | 0.447286 | 0.420605 |
| | symmetric_UGGUGU-UGGAGU | CAACUCCCUCGCGC | | CAGCCACA | | | | |
| | −6_3-3_bulge-symmetric_CAU-CAU | AACUUUGAACUGUA | | ACTCCCTCG | | | | |
| | 0_1-1_mismatch_A-C | ACUUUCAUGCAUAG | | CGCAACTTT | | | | |
| | 6_1-1_mismatch_G-G | AUCCACGGCUACAU | | GAACTGTA | | | | |
| | 10_1-1_mismatch_U-C | AAUUCCUUUUGGAG | | ACTTTCATG | | | | |
| | 19_1-2_bulge-asymmetric_G-AA | UCACUGUCGUCGAA | | CATAGATCC | | | | |
| | 22_1-2_bulge-asymmetric_U-CU | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 129 | −18_6−6_internal_loop- | CUCAGCAGCAGCCA | 568 | CTCAGCAG | 0.488543 | 0.488543 | 0.477455 | 0.394975 |
| | symmetric_UGGUGU-UGGAGU | CAACUCCCUCGCGC | | CAGCCACA | | | | |
| | −6_3-3_bulge-symmetric_CAU-CAU | AACUUCGAAACGCC | | ACTCCCTCG | | | | |
| | 0_1-1_mismatch_A-C | UUUCAUGCAUAGAU | | CGCAACTTC | | | | |
| | 6_1-1_mismatch_G-G | CCACGGCUACAUAA | | GAAACGCC | | | | |
| | 10_1-1_mismatch_U-C | UUCCUUUUGGAGUC | | TTTCATGCA | | | | |
| | 20_2-2_bulge-symmetric_AC-CG | ACUGUCGUCGAAUG | | TAGATCCAC | | | | |
| | 26_1-1_mismatch_A-C | GC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 130 | −18_6−6_internal_loop- | CUCAGCAGCAGCCA | 569 | CTCAGCAG | 0.479257 | 0.479257 | 0.440091 | 0.425876 |
| | symmetric_UGGUGU-UGGAGU | CAACUCCCUCGCGC | | CAGCCACA | | | | |
| | −6_3-3_bulge-symmetric_CAU-CAU | AACUUUGUAGAUUU | | ACTCCCTCG | | | | |
| | 0_1-1_mismatch_A-C | CAUGCAUAGAUCCA | | CGCAACTTT | | | | |
| | 6_1-1_mismatch_G-G | CGGCUACAUAAUUC | | GTAGATTTC | | | | |
| | 10_1-1_mismatch_U-C | CUUUUGGAGUCACU | | ATGCATAG | | | | |
| | 18_3-1_bulge-asymmetric_GGA-A | GUCGUCGAAUGGC | | ATCCACGG | | | | |
| | 23_2-1_bulge-asymmetric_UU-U | | | CTACATAAT | | | | |
| | 30_6−6_internal_loop- | | | TCCTTTTGG | | | | |
| | symmetric_GCCAAG-GCGCAA | | | AGTCACTGT | | | | |
| | | | | CGTCGAAT | | | | |
| | | | | GGC | | | | |
| 131 | −18_6−6_internal_loop- | CUCAGCAGCAGCCA | 570 | CTCAGCAG | 0.441079 | 0.441079 | 0.450558 | 0.407868 |
| | symmetric_UGGUGU-UGGAGU | CAACUCCCUCGCGC | | CAGCCACA | | | | |
| | −6_3-3_bulge-symmetric_CAU-CAU | AACUUUGAAUCGUC | | ACTCCCTCG | | | | |
| | 0_1-1_mismatch_A-C | CGAUUCAUGCAUAG | | CGCAACTTT | | | | |
| | 6_1-1_mismatch_G-G | AUCCACGGCUACAU | | GAATCGTCC | | | | |
| | 10_1-1_mismatch_U-C | AAUUCCUUUUGGAG | | GATTCATGC | | | | |
| | 17_1-2_bulge-asymmetric_A-GA | UCACUGUCGUCGAA | | ATAGATCC | | | | |
| | 22_1-2_bulge-asymmetric_U-UC | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 132 | −6_6−6_internal_loop- | CUCAGCAGCAGCCA | 571 | CTCAGCAG | 0.430839 | 0.430839 | 0.443099 | 0.384286 |
| | symmetric_AUUCAU-UCAUAC | CAACUCCCUCGCGC | | CAGCCACA | | | | |
| | 0_1-1_mismatch_A-C | AACUUUGACUAUGG | | ACTCCCTCG | | | | |
| | 9_3-3_bulge-symmetric_UUC-CCU | GGUUUCAUCCUUAC | | CGCAACTTT | | | | |
| | 18_3-3_bulge-symmetric_GGA-GGG | AUCCACGGCUAUCA | | GACTATGG | | | | |
| | 22_2-4_bulge-asymmetric_UU-CUAU | UACUCCUUUACACC | | GGTTTCATC | | | | |
| | 30_6−6_internal_loop- | ACACUGUCGUCGAA | | CTTACATCC | | | | |
| | symmetric_GCCAAG-GCGCAA | UGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 133 | −18_6−6_internal_loop- | CUCAGCAGCAGCCA | 572 | CTCAGCAG | 0.497958 | 0.497958 | 0.470128 | 0.390688 |
| | symmetric_UGGUGU-UGGAGU | CAACUCCCUCGCGC | | CAGCCACA | | | | |
| | −6_3-3_bulge-symmetric_CAU-CAU | AACUUUGCUAAACC | | ACTCCCTCG | | | | |
| | 0_1-1_mismatch_A-C | CUUUCAUGCAUAGA | | CGCAACTTT | | | | |
| | 6_1-1_mismatch_G-G | UCCACGGCUACAUA | | GCTAAACC | | | | |
| | 10_1-1_mismatch_U-C | AUUCCUUUUGGAGU | | CTTTCATGC | | | | |
| | 20_2-2_bulge-symmetric_AC-AC | CACUGUCGUCGAAU | | ATAGATCC | | | | |
| | 24_1-2_bulge-asymmetric_U-CU | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 134 | −6_6−6_internal_loop- | CUCAGCAGCAGCCA | 573 | CTCAGCAG | 0.401224 | 0.401224 | 0.486877 | 0.375039 |
| | symmetric_AUUCAU-UCAUAC | CAACUCCCUCGCGC | | CAGCCACA | | | | |
| | 0_1-1_mismatch_A-C | AACUGCCAAAGGAG | | ACTCCCTCG | | | | |
| | 8_3-3_bulge-symmetric_AUU-CGC | UUUCAUGCGCACAU | | CGCAACTG | | | | |
| | 18_3-3_bulge-symmetric_GGA-GAG | CCACGGCUAUCAUA | | CCAAAGGA | | | | |
| | 25_3-3_bulge-symmetric_CAA-GCC | CUCCUUUACACCAC | | GTTTCATGC | | | | |
| | 30_6−6_internal_loop- | ACUGUCGUCGAAUG | | GCACATCC | | | | |
| | symmetric_GCCAAG-GCGCAA | GC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 135 | −6_6−6_internal_loop- | CUCAGCAGCAGCCA | 574 | CTCAGCAG | 0.408902 | 0.408902 | 0.450957 | 0.358472 |
| | symmetric_AUUCAU-UCAUAC | CAACUCCCUCGCGC | | CAGCCACA | | | | |
| | 0_1-1_mismatch_A-C | AACUUAACAAGCGG | | ACTCCCTCG | | | | |
| | 9_2-4_bulge-asymmetric_UU-UCGC | UUUCAUGUCGCUAC | | CGCAACTTA | | | | |
| | 18_3-3_bulge-symmetric_GGA-CGG | AUCCACGGCUAUCA | | ACAAGCGG | | | | |
| | 24_3-3_bulge-symmetric_UCA-AAC | UACUCCUUUACACC | | TTTCATGTC | | | | |
| | 30_6−6_internal_loop- | ACACUGUCGUCGAA | | GCTACATCC | | | | |
| | symmetric_GCCAAG-GCGCAA | UGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 136 | −18_6−6_internal_loop- | CUCAGCAGCAGCCA | 575 | CTCAGCAG | 0.459503 | 0.459503 | 0.438881 | 0.399572 |
| | symmetric_UGGUGU-UGGAGU | CAACUCCCUCGCGC | | CAGCCACA | | | | |
| | −6_3-3_bulge-symmetric_CAU-CAU | AACUUUGAAAGUCA | | ACTCCCTCG | | | | |
| | 0_1-1_mismatch_A-C | UUCAUGCAUAGACA | | CGCAACTTT | | | | |
| | 3_2-2_bulge-symmetric_GA-CA | CACGGCUACAUAAU | | GAAAGTCA | | | | |
| | 6_1-1_mismatch_G-G | UCCUUUUGGAGUCA | | TTCATGCAT | | | | |
| | 10_1-1_mismatch_U-C | CUGUCGUCGAAUGG | | AGACACAC | | | | |
| | 17_2-1_bulge-asymmetric_AG-A | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 137 | −18_6−6_internal_loop- | CUCAGCAGCAGCCA | 576 | CTCAGCAG | 0.508859 | 0.508859 | 0.438869 | 0.426741 |
| | symmetric_UGGUGU-UGGAGU | CAACUCCCUCGCGC | | CAGCCACA | | | | |
| | −6_3-3_bulge-symmetric_CAU-CAU | AACUUUGACUCACA | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GACTCACAT | | | | |
| | 10_1-1_mismatch_U-C | CCUUUUGGAGUCAC | | CATGCATA | | | | |
| | 16_3-3_bulge-symmetric_AAG-ACA | UGUCGUCGAAUGGC | | GATCCACG | | | | |
| | 21_3-1_bulge-asymmetric_CUU-C | | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 138 | −6_6−6_internal_loop- | CUCAGCAGCAGCCA | 577 | CTCAGCAG | 0.409945 | 0.409945 | 0.4459 | 0.373056 |
| | symmetric_AUUCAU-UCAUAC | CAACUCCCUCGCGC | | CAGCCACA | | | | |
| | 0_1-1_mismatch_A-C | AACUUUGAAAGUGA | | ACTCCCTCG | | | | |
| | 6_1-1_mismatch_G-G | UUUCAUGAAUAGAU | | CGCAACTTT | | | | |
| | 18_2-2_bulge-symmetric_GG-GA | CCACGGCUAUCAUA | | GAAAGTGA | | | | |
| | 30_6−6_internal_loop- | CUCCUUUACACCAC | | TTTCATGAA | | | | |
| | symmetric_GCCAAG-GCGCAA | ACUGUCGUCGAAUG | | TAGATCCAC | | | | |
| | | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 139 | −6_6−6_internal_loop- | CUCAGCAGCAGCCA | 578 | CTCAGCAG | 0.403854 | 0.403854 | 0.443973 | 0.380163 |
| | symmetric_AUUCAU-UCAUAC | CAACUCCCUCGCGC | | CAGCCACA | | | | |
| | 0_1-1_mismatch_A-C | AACGGGGAAAGUAG | | ACTCCCTCG | | | | |
| | 7_3-3_bulge-symmetric_UAU-UGC | UAUUUCAUGAUGCC | | CGCAACGG | | | | |
| | 18_2-4_bulge-asymmetric_GG-AGUA | AUCCACGGCUAUCA | | GGAAAGTA | | | | |
| | 26_3-3_bulge-symmetric_AAA-GGG | UACUCCUUUACACC | | GTATTTCAT | | | | |
| | 30_6−6_internal_loop- | ACACUGUCGUCGAA | | GATGCCATC | | | | |
| | symmetric_GCCAAG-GCGCAA | UGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 140 | −6_6−6_internal_loop- | CUCAGCAGCAGCCA | 579 | CTCAGCAG | 0.416093 | 0.416093 | 0.441365 | 0.383058 |
| | symmetric_AUUCAU-UCAUAC | CAACUCCCUCGCGC | | CAGCCACA | | | | |
| | 0_1-1_mismatch_A-C | AACUUCUCUAAGUG | | ACTCCCTCG | | | | |
| | 7_3-3_bulge-symmetric_UAU-CCU | GGUUCAUGACCUCA | | CGCAACTTC | | | | |
| | 17_3-3_bulge-symmetric_AGG-GGG | UCCACGGCUAUCAU | | TCTAAGTGG | | | | |
| | 24_3-4_bulge-asymmetric_UCA-CUCU | ACUCCUUUACACCA | | GTTCATGAC | | | | |
| | 30_6−6_internal_loop- | CACUGUCGUCGAAU | | CTCATCCAC | | | | |
| | symmetric_GCCAAG-GCGCAA | GGC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 141 | −6_6−6_internal_loop- | CUCAGCAGCAGCCA | 580 | CTCAGCAG | 0.42231 | 0.42231 | 0.447497 | 0.362968 |
| | symmetric_AUUCAU-UCAUAC | CAACUCCCUCGCGC | | CAGCCACA | | | | |
| | 0_1-1_mismatch_A-C | AACUGUGAAAGUCG | | ACTCCCTCG | | | | |
| | 9_2-2_bulge-symmetric_UU-CU | UCAUGCUUACAUCC | | CGCAACTGT | | | | |
| | 16_3-1_bulge-asymmetric_AAG-G | ACGGCUAUCAUACU | | GAAAGTCG | | | | |
| | 27_1-1_mismatch_A-G | CCUUUACACCACAC | | TCATGCTTA | | | | |
| | 30_6−6_internal_loop- | UGUCGUCGAAUGGC | | CATCCACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 142 | −18_6−6_internal_loop- | CUCAGCAGCAGCCA | 581 | CTCAGCAG | 0.449995 | 0.449995 | 0.431323 | 0.405684 |
| | symmetric_UGGUGU-UGGAGU | CAACUCCCUCGCGC | | CAGCCACA | | | | |
| | −6_3-3_bulge-symmetric_CAU-CAU | AACUUUGUAGUCAU | | ACTCCCTCG | | | | |
| | 0_1-1_mismatch_A-C | UCAUGCAUAGAUCC | | CGCAACTTT | | | | |
| | 6_1-1_mismatch_G-G | ACGGCUACAUAAUU | | GTAGTCATT | | | | |
| | 10_1-1_mismatch_U-C | CCUUUUGGAGUCAC | | CATGCATA | | | | |
| | 17_2-1_bulge-asymmetric_AG-A | UGUCGUCGAAUGGC | | GATCCACG | | | | |
| | 23_2-1_bulge-asymmetric_UU-U | | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 143 | −6_6−6_internal_loop- | CUCAGCAGCAGCCA | 582 | CTCAGCAG | 0.392427 | 0.392427 | 0.467475 | 0.381986 |
| | symmetric_AUUCAU-UCAUAC | CAACUCCCUCGCGC | | CAGCCACA | | | | |
| | 0_1-1_mismatch_A-C | AACGAAGAACCCGC | | ACTCCCTCG | | | | |
| | 8_3-3_bulge-symmetric_AUU-CUG | CUUUCAUGCUGACA | | CGCAACGA | | | | |
| | 20_3-4_bulge-asymmetric_ACU-CCCG | UCCACGGCUAUCAU | | AGAACCCG | | | | |
| | 26_3-3_bulge-symmetric_AAA-GAA | ACUCCUUUACACCA | | CCTTTCATG | | | | |
| | 30_6−6_internal_loop- | CACUGUCGUCGAAU | | CTGACATCC | | | | |
| | symmetric_GCCAAG-GCGCAA | GGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 144 | −6_6−6_internal_loop- | CUCAGCAGCAGCCA | 583 | CTCAGCAG | 0.408317 | 0.408317 | 0.445719 | 0.384523 |
| | symmetric_AUUCAU-UCAUAC | CAACUCCCUCGCGC | | CAGCCACA | | | | |
| | 0_1-1_mismatch_A-C | AACUUCUAUAAAGG | | ACTCCCTCG | | | | |
| | 4_3-3_bulge-symmetric_AUG-ACC | AAUUUCAUGAAUAA | | CGCAACTTC | | | | |
| | 18_3-3_bulge-symmetric_GGA-GAA | CCCCACGGCUAUCA | | TATAAAGG | | | | |
| | 25_2-4_bulge-asymmetric_CA-CUAU | UACUCCUUUACACC | | AATTTCATG | | | | |
| | 30_6−6_internal_loop- | ACACUGUCGUCGAA | | AATAACCC | | | | |
| | symmetric_GCCAAG-GCGCAA | UGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 145 | −18_6−6_internal_loop- | CUCAGCAGCAGCCA | 584 | CTCAGCAG | 0.396728 | 0.396728 | 0.475444 | 0.406196 |
| | symmetric_UGGUGU-UGGAGU | CAACUCCCUCGCGC | | CAGCCACA | | | | |
| | −6_3-3_bulge-symmetric_CAU-CAU | AACUUUGAAAGGGG | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGGGG | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_3-3_bulge-symmetric_GGA-GGG | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 146 | −18_6−6_internal_loop- | CUCAGCAGCAGCCA | 585 | CTCAGCAG | 0.481035 | 0.481035 | 0.463079 | 0.389024 |
| | symmetric_UGGUGU-UGGAGU | CAACUCCCUCGCGC | | CAGCCACA | | | | |
| | −6_3-3_bulge-symmetric_CAU-CAU | AACUUUGAUUGUCC | | ACTCCCTCG | | | | |
| | 0_1-1_mismatch_A-C | UGUCAUGCAUAGAU | | CGCAACTTT | | | | |
| | 6_1-1_mismatch_G-G | CCACGGCUACAUAA | | GATTGTCCT | | | | |
| | 10_1-1_mismatch_U-C | UUCCUUUUGGAGUC | | GTCATGCAT | | | | |
| | 16_1-1_mismatch_A-G | ACUGUCGUCGAAUG | | AGATCCAC | | | | |
| | 22_2-2_bulge-symmetric_UU-UU | GC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 147 | −18_6−6_internal_loop- | CUCAGCAGCAGCCA | 586 | CTCAGCAG | 0.500681 | 0.500681 | 0.437085 | 0.419861 |
| | symmetric_UGGUGU-UGGAGU | CAACUCCCUCGCGC | | CAGCCACA | | | | |
| | −6_3-3_bulge-symmetric_CAU-CAU | AACUUUGCGUCCUG | | ACTCCCTCG | | | | |
| | 0_1-1_mismatch_A-C | AUGCAUAGAUCCAC | | CGCAACTTT | | | | |
| | 6_1-1_mismatch_G-G | GGCUACAUAAUUCC | | GCGTCCTGA | | | | |
| | 10_1-1_mismatch_U-C | UUUUGGAGUCACUG | | TGCATAGAT | | | | |
| | 14_3-1_bulge-asymmetric_GAA-G | UCGUCGAAUGGC | | CCACGGCT | | | | |
| | 22_3-1_bulge-asymmetric_UUU-C | | | ACATAATTC | | | | |
| | 30_6−6_internal_loop- | | | CTTTTGGAG | | | | |
| | symmetric_GCCAAG-GCGCAA | | | TCACTGTCG | | | | |
| | | | | TCGAATGG | | | | |
| | | | | C | | | | |
| 148 | −6_6−6_internal_loop- | CUCAGCAGCAGCCA | 587 | CTCAGCAG | 0.397278 | 0.397278 | 0.452363 | 0.374287 |
| | symmetric_AUUCAU-UCAUAC | CAACUCCCUCGCGC | | CAGCCACA | | | | |
| | 0_1-1_mismatch_A-C | AACUCGAAAAGUGA | | ACTCCCTCG | | | | |
| | 9_3-3_bulge-symmetric_UUC-UGC | CUUCAUUGCUACAU | | CGCAACTC | | | | |
| | 17_3-3_bulge-symmetric_AGG-GAC | CCACGGCUAUCAUA | | GAAAAGTG | | | | |
| | 25_3-3_bulge-symmetric_CAA-CGA | CUCCUUUACACCAC | | ACTTCATTG | | | | |
| | 30_6−6_internal_loop- | ACUGUCGUCGAAUG | | CTACATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 149 | −18_6−6_internal_loop- | CUCAGCAGCAGCCA | 588 | CTCAGCAG | 0.397191 | 0.397191 | 0.451206 | 0.402936 |
| | symmetric_UGGUGU-UGGAGU | CAACUCCCUCGCGC | | CAGCCACA | | | | |
| | −6_3-3_bulge-symmetric_CAU-CAU | AACUUUGAAAGUAA | | ACTCCCTCG | | | | |
| | 0_1-1_mismatch_A-C | CUUCAUGCAUAGAC | | CGCAACTTT | | | | |
| | 4_1-0_bulge-asymmetric_A- | CACGGCUACAUAAU | | GAAAGTAA | | | | |
| | 6_1-1_mismatch_G-G | UCCUUUUGGAGUCA | | CTTCATGCA | | | | |
| | 10_1-1_mismatch_U-C | CUGUCGUCGAAUGG | | TAGACCAC | | | | |
| | 17_3-3_bulge-symmetric_AGG-AAC | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 150 | −18_6−6_internal_loop- | CUCAGCAGCAGCCA | 589 | CTCAGCAG | 0.388194 | 0.388194 | 0.479389 | 0.406036 |
| | symmetric_UGGUGU-UGGAGU | CAACUCCCUCGCGC | | CAGCCACA | | | | |
| | −6_3-3_bulge-symmetric_CAU-CAU | AACUUUGAAAGUGG | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGTGG | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_2-2_bulge-symmetric_GG-GG | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 151 | −18_6−6_internal_loop- | CUCAGCAGCAGCCA | 590 | CTCAGCAG | 0.43694 | 0.43694 | 0.471482 | 0.374014 |
| | symmetric_UGGUGU-UGGAGU | CAACUCCCUCGCGC | | CAGCCACA | | | | |
| | −6_3-3_bulge-symmetric_CAU-CAU | AACUGUGAAAGUCC | | ACTCCCTCG | | | | |
| | 0_1-1_mismatch_A-C | UUUCAUGCAUAGAU | | CGCAACTGT | | | | |
| | 6_1-1_mismatch_G-G | CCACGGCUACAUAA | | GAAAGTCC | | | | |
| | 10_1-1_mismatch_U-C | UUCCUUUUGGAGUC | | TTTCATGCA | | | | |
| | 27_1-1_mismatch_A-G | ACUGUCGUCGAAUG | | TAGATCCAC | | | | |
| | 30_6−6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 152 | −18_6−6_internal_loop- | CUCAGCAGCAGCCA | 591 | CTCAGCAG | 0.468057 | 0.468057 | 0.485448 | 0.386627 |
| | symmetric_UGGUGU-UGGAGU | CAACUCCCUCGCGC | | CAGCCACA | | | | |
| | −6_3-3_bulge-symmetric_CAU-CAU | AACUUUGCUAAGUC | | ACTCCCTCG | | | | |
| | 0_1-1_mismatch_A-C | CUGUCAUGCAUAGA | | CGCAACTTT | | | | |
| | 6_1-1_mismatch_G-G | UCCACGGCUACAUA | | GCTAAGTCC | | | | |
| | 10_1-1_mismatch_U-C | AUUCCUUUUGGAGU | | TGTCATGCA | | | | |
| | 16_1-1_mismatch_A-G | CACUGUCGUCGAAU | | TAGATCCAC | | | | |
| | 24_1-2_bulge-asymmetric_U-CU | GGC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 153 | −6_6−6_internal_loop- | CUCAGCAGCAGCCA | 592 | CTCAGCAG | 0.386968 | 0.386968 | 0.475603 | 0.370481 |
| | symmetric_AUUCAU-UCAUAC | CAACUCCCUCGCGC | | CAGCCACA | | | | |
| | 0_1-1_mismatch_A-C | AACUUUCUCAAAGU | | ACTCCCTCG | | | | |
| | 9_1-1_mismatch_U-C | CCGUCAUGACUACA | | CGCAACTTT | | | | |
| | 16_2-1_bulge-asymmetric_AA-G | UCCACGGCUAUCAU | | CTCAAAGTC | | | | |
| | 25_1-3_bulge-asymmetric_C-CUC | ACUCCUUUACACCA | | CGTCATGAC | | | | |
| | 30_6−6_internal_loop- | CACUGUCGUCGAAU | | TACATCCAC | | | | |
| | symmetric_GCCAAG-GCGCAA | GGC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 154 | −18_6−6_internal_loop- | CUCAGCAGCAGCCA | 593 | CTCAGCAG | 0.436556 | 0.436556 | 0.443092 | 0.392954 |
| | symmetric_UGGUGU-UGGAGU | CAACUCCCUCGCGC | | CAGCCACA | | | | |
| | −6_3-3_bulge-symmetric_CAU-CAU | AACUUUGACCCUCC | | ACTCCCTCG | | | | |
| | 0_1-1_mismatch_A-C | GCGUUCAUGCAUAG | | CGCAACTTT | | | | |
| | 6_1-1_mismatch_G-G | AUCCACGGCUACAU | | GACCCTCCG | | | | |
| | 10_1-1_mismatch_U-C | AAUUCCUUUUGGAG | | CGTTCATGC | | | | |
| | 17_1-3_bulge-asymmetric_A-GCG | UCACUGUCGUCGAA | | ATAGATCC | | | | |
| | 21_3-3_bulge-symmetric_CUU-CCC | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 155 | −18_6−6_internal_loop- | CUCAGCAGCAGCCA | 594 | CTCAGCAG | 0.400062 | 0.400062 | 0.448712 | 0.400349 |
| | symmetric_UGGUGU-UGGAGU | CAACUCCCUCGCGC | | CAGCCACA | | | | |
| | −6_3-3_bulge-symmetric_CAU-CAU | AACUUUGAAAGGAC | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGGAC | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 19_2-2_bulge-symmetric_GA-GA | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 156 | −18_6−6_internal_loop- | CUCAGCAGCAGCCA | 595 | CTCAGCAG | 0.451541 | 0.451541 | 0.454843 | 0.386124 |
| | symmetric_UGGUGU-UGGAGU | CAACUCCCUCGCGC | | CAGCCACA | | | | |
| | −6_3-3_bulge-symmetric_CAU-CAU | AACUUUGUUUAAGU | | ACTCCCTCG | | | | |
| | 0_1-1_mismatch_A-C | CCUCUCAUGCAUAG | | CGCAACTTT | | | | |
| | 6_1-1_mismatch_G-G | AUCCACGGCUACAU | | GTTTAAGTC | | | | |
| | 10_1-1_mismatch_U-C | AAUUCCUUUUGGAG | | CTCTCATGC | | | | |
| | 16_1-1_mismatch_A-C | UCACUGUCGUCGAA | | ATAGATCC | | | | |
| | 24_1-3_bulge-asymmetric_U-UUU | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 157 | −18_6−6_internal_loop- | CUCAGCAGCAGCCA | 596 | CTCAGCAG | 0.471676 | 0.471676 | 0.433525 | 0.424654 |
| | symmetric_UGGUGU-UGGAGU | CAACUCCCUCGCGC | | CAGCCACA | | | | |
| | −6_3-3_bulge-symmetric_CAU-CAU | AACUUUGACGCUCC | | ACTCCCTCG | | | | |
| | 0_1-1_mismatch_A-C | CUCAUGCAUAGAUC | | CGCAACTTT | | | | |
| | 6_1-1_mismatch_G-G | CACGGCUACAUAAU | | GACGCTCCC | | | | |
| | 10_1-1_mismatch_U-C | UCCUUUUGGAGUCA | | TCATGCATA | | | | |
| | 16_2-1_bulge-asymmetric_AA-C | CUGUCGUCGAAUGG | | GATCCACG | | | | |
| | 21_3-3_bulge-symmetric_CUU-CGC | C | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 158 | −18_6−6_internal_loop- | CUCAGCAGCAGCCA | 597 | CTCAGCAG | 0.50252 | 0.50252 | 0.4316 | 0.443291 |
| | symmetric_UGGUGU-UGGAGU | CAACUCCCUCGCGC | | CAGCCACA | | | | |
| | −6_3-3_bulge-symmetric_CAU-CAU | AACUUUGACGCAGU | | ACTCCCTCG | | | | |
| | 0_1-1_mismatch_A-C | CGUCAUGCAUAGAU | | CGCAACTTT | | | | |
| | 6_1-1_mismatch_G-G | CCACGGCUACAUAA | | GACGCAGT | | | | |
| | 10_1-1_mismatch_U-C | UUCCUUUUGGAGUC | | CGTCATGCA | | | | |
| | 16_3-1_bulge-asymmetric_AAG-G | ACUGUCGUCGAAUG | | TAGATCCAC | | | | |
| | 23_1-3_bulge-asymmetric_U-CGC | GC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 159 | −18_6−6_internal_loop- | CUCAGCAGCAGCCA | 598 | CTCAGCAG | 0.455126 | 0.455126 | 0.424776 | 0.397757 |
| | symmetric_UGGUGU-UGGAGU | CAACUCCCUCGCGC | | CAGCCACA | | | | |
| | −6_3-3_bulge-symmetric_CAU-CAU | AACUUUGAAAGUGU | | ACTCCCTCG | | | | |
| | 0_1-1_mismatch_A-C | UCGUGCAUAGAUCC | | CGCAACTTT | | | | |
| | 6_1-1_mismatch_G-G | ACGGCUACAUAAUU | | GAAAGTGT | | | | |
| | 10_1-1_mismatch_U-C | CCUUUUGGAGUCAC | | TCGTGCATA | | | | |
| | 13_1-1_wobble_U-G | UGUCGUCGAAUGGC | | GATCCACG | | | | |
| | 17_3-1_bulge-asymmetric_AGG-G | | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 160 | −18_6−6_internal_loop- | CUCAGCAGCAGCCA | 599 | CTCAGCAG | 0.499617 | 0.499617 | 0.471561 | 0.384277 |
| | symmetric_UGGUGU-UGGAGU | CAACUCCCUCGCGC | | CAGCCACA | | | | |
| | −6_3-3_bulge-symmetric_CAU-CAU | AACUUUGUCAAGUC | | ACTCCCTCG | | | | |
| | 0_1-1_mismatch_A-C | CGUUCAUGCAUAGA | | CGCAACTTT | | | | |
| | 6_1-1_mismatch_G-G | UCCACGGCUACAUA | | GTCAAGTCC | | | | |
| | 10_1-1_mismatch_U-C | AUUCCUUUUGGAGU | | GTTCATGCA | | | | |
| | 17_1-1_mismatch_A-G | CACUGUCGUCGAAU | | TAGATCCAC | | | | |
| | 24_1-2_bulge-asymmetric_U-UC | GGC | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 161 | −6_6−6_internal_loop- | CUCAGCAGCAGCCA | 600 | CTCAGCAG | 0.396228 | 0.396228 | 0.427016 | 0.364645 |
| | symmetric_AUUCAU-UCAUAC | CAACUCCCUCGCGC | | CAGCCACA | | | | |
| | 0_1-1_mismatch_A-C | AACUUCAAGUGUUU | | ACTCCCTCG | | | | |
| | 6_2-2_bulge-symmetric_GU-CG | CAUGAAUCGAUCCA | | CGCAACTTC | | | | |
| | 18_2-1_bulge-asymmetric_GG-G | CGGCUAUCAUACUC | | AAGTGTTTC | | | | |
| | 24_3-1_bulge-asymmetric_UCA-C | CUUUACACCACACU | | ATGAATCG | | | | |
| | 30_6−6_internal_loop- | GUCGUCGAAUGGC | | ATCCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 162 | −6_6−6_internal_loop- | CUCAGCAGCAGCCA | 601 | CTCAGCAG | 0.386362 | 0.386362 | 0.470124 | 0.38089 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 4_1-1_mismatch_A-A | UUCAUGCAUACAAC | | CGCAACTTT | | | | |
| | 10_1-1_mismatch_U-C | CACGGCUAUCAUAC | | GAAAGTGT | | | | |
| | 18_2-1_bulge-asymmetric_GG-G | UCCUUUACACCACA | | TTCATGCAT | | | | |
| | 30_6−6_internal_loop- | CUGUCGUCGAAUGG | | ACAACCAC | | | | |
| | symmetric_GCCAAG-GCGCAA | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 163 | −18_6−6_internal_loop- | CUCAGCAGCAGCCA | 602 | CTCAGCAG | 0.466717 | 0.466717 | 0.455612 | 0.383268 |
| | symmetric_UGGUGU-UGGAGU | CAACUCCCUCGCGC | | CAGCCACA | | | | |
| | −6_3-3_bulge-symmetric_CAU-CAU | AACUUUGACCGUCC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GACCGTCCT | | | | |
| | 10_1-1_mismatch_U-C | UUCCUUUUGGAGUC | | TTCATGCAT | | | | |
| | 22_2-2_bulge-symmetric_UU-CC | ACUGUCGUCGAAUG | | AGATCCAC | | | | |
| | 30_6−6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 164 | −18_6−6_internal_loop- | CUCAGCAGCAGCCA | 603 | CTCAGCAG | 0.45349 | 0.45349 | 0.436059 | 0.425339 |
| | symmetric_UGGUGU-UGGAGU | CAACUCCCUCGCGC | | CAGCCACA | | | | |
| | −6_3-3_bulge-symmetric_CAU-CAU | AACUUUGAAAUUCC | | ACTCCCTCG | | | | |
| | 0_1-1_mismatch_A-C | CGAUUCAUGCAUAG | | CGCAACTTT | | | | |
| | 6_1-1_mismatch_G-G | AUCCACGGCUACAU | | GAAATTCCC | | | | |
| | 10_1-1_mismatch_U-C | AAUUCCUUUUGGAG | | GATTCATGC | | | | |
| | 17_1-3_bulge-asymmetric_A-CGA | UCACUGUCGUCGAA | | ATAGATCC | | | | |
| | 21_1-1_mismatch_C-U | UGGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 165 | −6_6−6_internal_loop- | CUCAGCAGCAGCCA | 604 | CTCAGCAG | 0.387059 | 0.387059 | 0.453959 | 0.362156 |
| | symmetric_AUUCAU-UCAUAC | CAACUCCCUCGCGC | | CAGCCACA | | | | |
| | 0_1-1_mismatch_A-C | AACUACAAAAGGGG | | ACTCCCTCG | | | | |
| | 9_3-3_bulge-symmetric_UUC-CCU | AUUCAUCCUUACAU | | CGCAACTA | | | | |
| | 17_4-4_bulge-symmetric_AGGA-GGGA | CCACGGCUAUCAUA | | CAAAAGGG | | | | |
| | 25_3-3_bulge-symmetric_CAA-ACA | CUCCUUUACACCAC | | GATTCATCC | | | | |
| | 30_6−6_internal_loop- | ACUGUCGUCGAAUG | | TTACATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 166 | −6_6−6_internal_loop- | CUCAGCAGCAGCCA | 605 | CTCAGCAG | 0.403949 | 0.403949 | 0.438894 | 0.383088 |
| | symmetric_AUUCAU-UCAUAC | CAACUCCCUCGCGC | | CAGCCACA | | | | |
| | 0_1-1_mismatch_A-C | AACUUUGCUUGGAU | | ACTCCCTCG | | | | |
| | 9_3-3_bulge-symmetric_UUC-UGC | GUUCAUUGCUACAU | | CGCAACTTT | | | | |
| | 17_4-4_bulge-symmetric_AGGA-GAUG | CCACGGCUAUCAUA | | GCTTGGATG | | | | |
| | 22_3-3_bulge-symmetric_UUU-CUU | CUCCUUUACACCAC | | TTCATTGCT | | | | |
| | 30_6−6_internal_loop- | ACUGUCGUCGAAUG | | ACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 167 | −6_6−6_internal_loop- | CUCAGCAGCAGCCA | 606 | CTCAGCAG | 0.40634 | 0.40634 | 0.448473 | 0.363219 |
| | symmetric_AUUCAU-UCAUAC | CAACUCCCUCGCGC | | CAGCCACA | | | | |
| | 0_1-1_mismatch_A-C | AACUUUGAAAGGUU | | ACTCCCTCG | | | | |
| | 2_1-1_wobble_G-U | UCAUGAAUAGAUCU | | CGCAACTTT | | | | |
| | 6_1-1_mismatch_G-G | ACGGCUAUCAUACU | | GAAAGGTT | | | | |
| | 18_3-1_bulge-asymmetric_GGA-G | CCUUUACACCACAC | | TCATGAATA | | | | |
| | 30_6−6_internal_loop- | UGUCGUCGAAUGGC | | GATCTACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 168 | −18_6−6_internal_loop- | CUCAGCAGCAGCCA | 607 | CTCAGCAG | 0.503936 | 0.503936 | 0.429163 | 0.428452 |
| | symmetric_UGGUGU-UGGAGU | CAACUCCCUCGCGC | | CAGCCACA | | | | |
| | −6_3-3_bulge-symmetric_CAU-CAU | AACUUUGAAACAGC | | ACTCCCTCG | | | | |
| | 0_1-1_mismatch_A-C | UUCCCAUGCAUAGA | | CGCAACTTT | | | | |
| | 6_1-1_mismatch_G-G | UCCACGGCUACAUA | | GAAACAGC | | | | |
| | 10_1-1_mismatch_U-C | AUUCCUUUUGGAGU | | TTCCCATGC | | | | |
| | 15_1-2_bulge-asymmetric_A-CC | CACUGUCGUCGAAU | | ATAGATCC | | | | |
| | 19_3-3_bulge-symmetric_GAC-CAG | GGC | | ACGGCTAC | | | | |
| | 30_6−6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 169 | −18_6−6_internal_loop- | CUCAGCAGCAGCCA | 608 | CTCAGCAG | 0.41691 | 0.41691 | 0.430103 | 0.389103 |
| | symmetric_UGGUGU-UGGAGU | CAACUCCCUCGCGC | | CAGCCACA | | | | |
| | −6_3-3_bulge-symmetric_CAU-CAU | AACUUUGAAAGUCC | | ACTCCCTCG | | | | |
| | 0_1-1_mismatch_A-C | UUAAUUGCAUAGAC | | CGCAACTTT | | | | |
| | 4_1-0_bulge-asymmetric_A- | CACGGCUACAUAAU | | GAAAGTCC | | | | |
| | 6_1-1_mismatch_G-G | UCCUUUUGGAGUCA | | TTAATTGCA | | | | |
| | 10_1-1_mismatch_U-C | CUGUCGUCGAAUGG | | TAGACCAC | | | | |
| | 13_3-3_bulge-symmetric_UGA-AAU | C | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 170 | −6_6−6_internal_loop- | CUCAGCAGCAGCCA | 609 | CTCAGCAG | 0.394419 | 0.394419 | 0.4464 | 0.362283 |
| | symmetric_AUUCAU-UCAUAC | CAACUCCCUCGCGC | | CAGCCACA | | | | |
| | 0_1-1_mismatch_A-C | AACUUUGUGUGAUU | | ACTCCCTCG | | | | |
| | 5_1-2_bulge-asymmetric_U-CC | UCAUGAAUACCCUC | | CGCAACTTT | | | | |
| | 18_2-2_bulge-symmetric_GG-GA | CACGGCUAUCAUAC | | GTGTGATTT | | | | |
| | 22_3-1_bulge-asymmetric_UUU-U | UCCUUUACACCACA | | CATGAATA | | | | |
| | 30_6−6_internal_loop- | CUGUCGUCGAAUGG | | CCCTCCACG | | | | |
| | symmetric_GCCAAG-GCGCAA | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 171 | −6_6−6_internal_loop- | CUCAGCAGCAGCCA | 610 | CTCAGCAG | 0.394314 | 0.394314 | 0.440563 | 0.371236 |
| | symmetric_AUUCAU-UCAUAC | CAACUCCCUCGCGC | | CAGCCACA | | | | |
| | 0_1-1_mismatch_A-C | AACGUCGACCCUCC | | ACTCCCTCG | | | | |
| | 7_1-4_bulge-asymmetric_U-UGAC | UUUCAUGAAUUGAC | | CGCAACGT | | | | |
| | 21_3-3_bulge-symmetric_CUU-CCC | CAUCCACGGCUAUC | | CGACCCTCC | | | | |
| | 26_3-3_bulge-symmetric_AAA-GUC | AUACUCCUUUACAC | | TTTCATGAA | | | | |
| | 30_6−6_internal_loop- | CACACUGUCGUCGA | | TTGACCATC | | | | |
| | symmetric_GCCAAG-GCGCAA | AUGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 172 | −18_6−6_internal_loop- | CUCAGCAGCAGCCA | 611 | CTCAGCAG | 0.421252 | 0.421252 | 0.424586 | 0.383216 |
| | symmetric_UGGUGU-UGGAGU | CAACUCCCUCGCGC | | CAGCCACA | | | | |
| | −6_3-3_bulge-symmetric_CAU-CAU | AACUUUGAAAGUAU | | ACTCCCTCG | | | | |
| | 0_1-1_mismatch_A-C | UCAUGCAUAGAUCU | | CGCAACTTT | | | | |
| | 2_1-1_wobble_G-U | ACGGCUACAUAAUU | | GAAAGTAT | | | | |
| | 6_1-1_mismatch_G-G | CCUUUUGGAGUCAC | | TCATGCATA | | | | |
| | 10_1-1_mismatch_U-C | UGUCGUCGAAUGGC | | GATCTACG | | | | |
| | 17_3-1_bulge-asymmetric_AGG-A | | | GCTACATA | | | | |
| | 30_6−6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 173 | −6_6−6_internal_loop- | CUCAGCAGCAGCCA | 612 | CTCAGCAG | 0.386881 | 0.386881 | 0.468261 | 0.356016 |
| | symmetric_AUUCAU-UCAUAC | CAACUCCCUCGCGC | | CAGCCACA | | | | |
| | 0_1-1_mismatch_A-C | AACGGUGAAAGACC | | ACTCCCTCG | | | | |
| | 4_1-0_bulge-asymmetric_A- | UUUCAUGAAUACAC | | CGCAACGG | | | | |
| | 20_1-1_mismatch_A-A | CACGGCUAUCAUAC | | TGAAAGAC | | | | |
| | 27_2-2_bulge-symmetric_AA-GG | UCCUUUACACCACA | | CTTTCATGA | | | | |
| | 30_6−6_internal_loop- | CUGUCGUCGAAUGG | | ATACACCA | | | | |
| | symmetric_GCCAAG-GCGCAA | C | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 174 | −6_6−6_internal_loop- | CUCAGCAGCAGCCA | 613 | CTCAGCAG | 0.38319 | 0.38319 | 0.441349 | 0.384672 |
| | symmetric_AUUCAU-UCAUAC | CAACUCCCUCGCGC | | CAGCCACA | | | | |
| | 0_1-1_mismatch_A-C | AACUUUCAGUGAUU | | ACTCCCTCG | | | | |
| | 6_1-1_mismatch_G-G | UCAUGAAUAGAUCC | | CGCAACTTT | | | | |
| | 18_2-2_bulge-symmetric_GG-GA | ACGGCUAUCAUACU | | CAGTGATTT | | | | |
| | 23_3-1_bulge-asymmetric_UUC-C | CCUUUACACCACAC | | CATGAATA | | | | |
| | 30_6−6_internal_loop- | UGUCGUCGAAUGGC | | GATCCACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 175 | −18_6−6_internal_loop- | CUCAGCAGCAGCCA | 614 | CTCAGCAG | 0.505995 | 0.505995 | 0.437463 | 0.413175 |
| | symmetric_UGGUGU-UGGAGU | CAACUCCCUCGCGC | | CAGCCACA | | | | |
| | −6_3-3_bulge-symmetric_CAU-CAU | AACUUUGAACCCUG | | ACTCCCTCG | | | | |
| | 0_1-1_mismatch_A-C | GAAUGCAUAGAUCC | | CGCAACTTT | | | | |
| | 6_1-1_mismatch_G-G | ACGGCUACAUAAUU | | GAACCCTG | | | | |
| | 10_1-1_mismatch_U-C | CCUUUUGGAGUCAC | | GAATGCAT | | | | |
| | 14_3-3_bulge-symmetric_GAA-GGA | UGUCGUCGAAUGGC | | AGATCCAC | | | | |
| | 20_3-1_bulge-asymmetric_ACU-C | | | GGCTACAT | | | | |
| | 30_6−6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 176 | −18_6−6_internal_loop- | CUCAGCAGCAGCCA | 615 | CTCAGCAG | 0.464709 | 0.464709 | 0.452543 | 0.377105 |
| | symmetric_UGGUGU-UGGAGU | CAACUCCCUCGCGC | | CAGCCACA | | | | |
| | -6_3-3_bulge-symmetric_CAU-CAU | AACUUUGUAGUCCU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GTAGTCCTT | | | | |
| | 10_1-1_mismatch_U-C | UCCUUUUGGAGUCA | | TCATGCATA | | | | |
| | 23_2-1_bulge-asymmetric_UU-U | CUGUCGUCGAAUGG | | GATCCACG | | | | |
| | 30_6-6_internal_loop- | C | | GCTACATA | | | | |
| | symmetric_GCCAAG-GCGCAA | | | ATTCCTTTT | | | | |
| | | | | GGAGTCAC | | | | |
| | | | | TGTCGTCGA | | | | |
| | | | | ATGGC | | | | |
| 177 | −18_6-6_internal_loop- | CUCAGCAGCAGCCA | 616 | CTCAGCAG | 0.429542 | 0.429542 | 0.451361 | 0.379 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGA | | ACTCCCTCG | | | | |
| | 0_1-1_mismatch_A-C | UUUCAUGCAUAGAC | | CGCAACTTT | | | | |
| | 3_2-2_bulge-symmetric_GA-CA | ACACGGCUACAUAA | | GAAAGTGA | | | | |
| | 6_1-1_mismatch_G-G | UUCCUUUUGGAGUC | | TTTCATGCA | | | | |
| | 10_1-1_mismatch_U-C | ACUGUCGUCGAAUG | | TAGACACA | | | | |
| | 18_2-2_bulge-symmetric_GG-GA | GC | | CGGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 178 | −18_6-6_internal_loop- | CUCAGCAGCAGCCA | 617 | CTCAGCAG | 0.504517 | 0.504517 | 0.424199 | 0.443949 |
| | symmetric_UGGUGU-UGGAGU | CAACUCCCUCGCGC | | CAGCCACA | | | | |
| | -6_3-3_bulge-symmetric_CAU-CAU | AACUUUGACUCCUA | | ACTCCCTCG | | | | |
| | 0_1-1_mismatch_A-C | GCAUGCAUAGAUCC | | CGCAACTTT | | | | |
| | 6_1-1_mismatch_G-G | ACGGCUACAUAAUU | | GACTCCTAG | | | | |
| | 10_1-1_mismatch_U-C | CCUUUUGGAGUCAC | | CATGCATA | | | | |
| | 15_2-2_bulge-symmetric_AA-AG | UGUCGUCGAAUGGC | | GATCCACG | | | | |
| | 21_3-1_bulge-asymmetric_CUU-C | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 179 | -6_6-6_internal_loop- | CUCAGCAGCAGCCA | 618 | CTCAGCAG | 0.404381 | 0.404381 | 0.416624 | 0.371128 |
| | symmetric_AUUCAU-UCAUAC | CAACUCCCUCGCGC | | CAGCCACA | | | | |
| | 0_1-1_mismatch_A-C | AACUGCCAAAGUGG | | ACTCCCTCG | | | | |
| | 8_3-3_bulge-symmetric_AUU-UCC | GGUUUCAUGUCCAC | | CGCAACTG | | | | |
| | 18_2-4_bulge-asymmetric_GG-GGGG | AUCCACGGCUAUCA | | CCAAAGTG | | | | |
| | 25_3-3_bulge-symmetric_CAA-GCC | UACUCCUUUACACC | | GGGTTTCAT | | | | |
| | 30_6-6_internal_loop- | ACACUGUCGUCGAA | | GTCCACATC | | | | |
| | symmetric_GCCAAG-GCGCAA | UGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 180 | -6_6-6_internal_loop- | CUCAGCAGCAGCCA | 619 | CTCAGCAG | 0.405819 | 0.405819 | 0.439351 | 0.379461 |
| | symmetric_AUUCAU-UCAUAC | CAACUCCCUCGCGC | | CAGCCACA | | | | |
| | 0_1-1_mismatch_A-C | AACUUUGAAAGUCG | | ACTCCCTCG | | | | |
| | 4_1-1_mismatch_A-A | UCAUGUUACAACCA | | CGCAACTTT | | | | |
| | 9_2-1_bulge-asymmetric_UU-U | CGGCUAUCAUACUC | | GAAAGTCG | | | | |
| | 16_3-1_bulge-asymmetric_AAG-G | CUUUACACCACACU | | TCATGTTAC | | | | |
| | 30_6-6_internal_loop- | GUCGUCGAAUGGC | | AACCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 181 | −18_6-6_internal_loop- | CUCAGCAGCAGCCA | 620 | CTCAGCAG | 0.450808 | 0.450808 | 0.453206 | 0.368384 |
| | symmetric_UGGUGU-UGGAGU | CAACUCCCUCGCGC | | CAGCCACA | | | | |
| | -6_3-3_bulge-symmetric_CAU-CAU | AACUUUGUUAAGUC | | ACTCCCTCG | | | | |
| | 0_1-1_mismatch_A-C | CUUUCGUGCAUAGA | | CGCAACTTT | | | | |
| | 6_1-1_mismatch_G-G | UCCACGGCUACAUA | | GTTAAGTCC | | | | |
| | 10_1-1_mismatch_U-C | AUUCCUUUUGGAGU | | TTTCGTGCA | | | | |
| | 13_1-1_wobble_U-G | CACUGUCGUCGAAU | | TAGATCCAC | | | | |
| | 24_1-2_bulge-asymmetric_U-UU | GGC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 182 | −18_6-6_internal_loop- | CUCAGCAGCAGCCA | 621 | CTCAGCAG | 0.46462 | 0.46462 | 0.461551 | 0.376478 |
| | symmetric_UGGUGU-UGGAGU | CAACUCCCUCGCGC | | CAGCCACA | | | | |
| | -6_3-3_bulge-symmetric_CAU-CAU | AACUUUGUAGUCGU | | ACTCCCTCG | | | | |
| | 0_1-1_mismatch_A-C | UUCAUGCAUAGAUC | | CGCAACTTT | | | | |
| | 6_1-1_mismatch_G-G | CACGGCUACAUAAU | | GTAGTCGTT | | | | |
| | 10_1-1_mismatch_U-C | UCCUUUUGGAGUCA | | TCATGCATA | | | | |
| | 18_1-1_mismatch_G-G | CUGUCGUCGAAUGG | | GATCCACG | | | | |
| | 23_2-1_bulge-asymmetric_UU-U | C | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 183 | -6_6-6_internal_loop- | CUCAGCAGCAGCCA | 622 | CTCAGCAG | 0.389042 | 0.389042 | 0.439221 | 0.358874 |
| | symmetric_AUUCAU-UCAUAC | CAACUCCCUCGCGC | | CAGCCACA | | | | |
| | 0_1-1_mismatch_A-C | AACUUUGAAAGUAU | | ACTCCCTCG | | | | |
| | 6_1-2_bulge-asymmetric_G-AG | UCCAUGAAUAAGAU | | CGCAACTTT | | | | |
| | 15_1-1_mismatch_A-C | CCACGGCUAUCAUA | | GAAAGTAT | | | | |
| | 18_2-1_bulge-asymmetric_GG-A | CUCCUUUACACCAC | | TCCATGAAT | | | | |
| | 30_6-6_internal_loop- | ACUGUCGUCGAAUG | | AAGATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 184 | −18_6-6_internal_loop- | CUCAGCAGCAGCCA | 623 | CTCAGCAG | 0.379853 | 0.379853 | 0.459579 | 0.394456 |
| | symmetric_UGGUGU-UGGAGU | CAACUCCCUCGCGC | | CAGCCACA | | | | |
| | -6_3-3_bulge-symmetric_CAU-CAU | AACUUUGAAAGGAG | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGGAG | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_3-3_bulge-symmetric_GGA-GAG | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 185 | -6_6-6_internal_loop- | CUCAGCAGCAGCCA | 624 | CTCAGCAG | 0.377193 | 0.377193 | 0.459665 | 0.380596 |
| | symmetric_AUUCAU-UCAUAC | CAACUCCCUCGCGC | | CAGCCACA | | | | |
| | 0_1-1_mismatch_A-C | AACUUGUUAAGCAU | | ACTCCCTCG | | | | |
| | 9_3-3_bulge-symmetric_UUC-CAC | AUUUCAUCACUACA | | CGCAACTTG | | | | |
| | 18_3-4_bulge-asymmetric_GGA-CAUA | UCCACGGCUAUCAU | | TTAAGCATA | | | | |
| | 24_3-3_bulge-symmetric_UCA-GUU | ACUCCUUUACACCA | | TTTCATCAC | | | | |
| | 30_6-6_internal_loop- | CACUGUCGUCGAAU | | TACATCCAC | | | | |
| | symmetric_GCCAAG-GCGCAA | GGC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 186 | -6_6-6_internal_loop- | CUCAGCAGCAGCCA | 625 | CTCAGCAG | 0.373585 | 0.373585 | 0.470273 | 0.386827 |
| | symmetric_AUUCAU-UCAUAC | CAACUCCCUCGCGC | | CAGCCACA | | | | |
| | 0_1-1_mismatch_A-C | AACUUGUCAAGAGA | | ACTCCCTCG | | | | |
| | 9_3-3_bulge-symmetric_UUC-CUU | UUUCAUCUUUACAU | | CGCAACTTG | | | | |
| | 18_3-3_bulge-symmetric_GGA-AGA | CCACGGCUAUCAUA | | TCAAGAGA | | | | |
| | 24_3-3_bulge-symmetric_UCA-GUC | CUCCUUUACACCAC | | TTTCATCTT | | | | |
| | 30_6-6_internal_loop- | ACUGUCGUCGAAUG | | TACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 187 | -6_6-6_internal_loop- | CUCAGCAGCAGCCA | 626 | CTCAGCAG | 0.386566 | 0.386566 | 0.438147 | 0.365031 |
| | symmetric_AUUCAU-UCAUAC | CAACUCCCUCGCGC | | CAGCCACA | | | | |
| | 0_1-1_mismatch_A-C | AACUUUGAAAGGCU | | ACTCCCTCG | | | | |
| | 4_1-0_bulge-asymmetric_A- | UUCAUGAAUGCACC | | CGCAACTTT | | | | |
| | 7_1-1_wobble_U-G | ACGGCUAUCAUACU | | GAAAGGCT | | | | |
| | 19_2-1_bulge-asymmetric_GA-G | CCUUUACACCACAC | | TTCATGAAT | | | | |
| | | UGUCGUCGAAUGGC | | GCACCACG | | | | |
| | 30_6-6_internal_loop- | | | GCTATCATA | | | | |
| | symmetric_GCCAAG-GCGCAA | | | CTCCTTTAC | | | | |
| | | | | ACCACACT | | | | |
| | | | | GTCGTCGA | | | | |
| | | | | ATGGC | | | | |
| 188 | −18_6-6_internal_loop- | CUCAGCAGCAGCCA | 627 | CTCAGCAG | 0.507803 | 0.507803 | 0.414845 | 0.423386 |
| | symmetric_UGGUGU-UGGAGU | CAACUCCCUCGCGC | | CAGCCACA | | | | |
| | -6_3-3_bulge-symmetric_CAU-CAU | AACUUUGAACUACC | | ACTCCCTCG | | | | |
| | 0_1-1_mismatch_A-C | UACAUGCAUAGAUC | | CGCAACTTT | | | | |
| | 6_1-1_mismatch_G-G | CACGGCUACAUAAU | | GAACTACCT | | | | |
| | 10_1-1_mismatch_U-C | UCCUUUUGGAGUCA | | ACATGCAT | | | | |
| | 15_2-1_bulge-asymmetric_AA-A | CUGUCGUCGAAUGG | | AGATCCAC | | | | |
| | 20_3-3_bulge-symmetric_ACU-CUA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 189 | −18_6-6_internal_loop- | CUCAGCAGCAGCCA | 628 | CTCAGCAG | 0.501031 | 0.501031 | 0.486453 | 0.375068 |
| | symmetric_UGGUGU-UGGAGU | CAACUCCCUCGCGC | | CAGCCACA | | | | |
| | -6_3-3_bulge-symmetric_CAU-CAU | AACUUUGCAUAAGC | | ACTCCCTCG | | | | |
| | 0_1-1_mismatch_A-C | ACUUUCAUGCAUAG | | CGCAACTTT | | | | |
| | 6_1-1_mismatch_G-G | AUCCACGGCUACAU | | GCATAAGC | | | | |
| | 10_1-1_mismatch_U-C | AAUUCCUUUUGGAG | | ACTTTCATG | | | | |
| | 19_2-2_bulge-symmetric_GA-CA | UCACUGUCGUCGAA | | CATAGATCC | | | | |
| | 24_1-3_bulge-asymmetric_U-CAU | UGGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 190 | -6_6-6_internal_loop- | CUCAGCAGCAGCCA | 629 | CTCAGCAG | 0.408965 | 0.408965 | 0.412749 | 0.36468 |
| | symmetric_AUUCAU-UCAUAC | CAACUCCCUCGCGC | | CAGCCACA | | | | |
| | 0_1-1_mismatch_A-C | AACUUUGAAUGCUG | | ACTCCCTCG | | | | |
| | 8_3-3_bulge-symmetric_AUU-CUC | UGGAUUCAUGCUCA | | CGCAACTTT | | | | |
| | 17_3-3_bulge-symmetric_AGG-GGA | CAUCCACGGCUAUC | | GAATGCTGT | | | | |
| | 22_1-4_bulge-asymmetric_U-UGCU | AUACUCCUUUACAC | | GGATTCATG | | | | |
| | 30_6-6_internal_loop- | CACACUGUCGUCGA | | CTCACATCC | | | | |
| | symmetric_GCCAAG-GCGCAA | AUGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 191 | −18_6-6_internal_loop- | CUCAGCAGCAGCCA | 630 | CTCAGCAG | 0.380749 | 0.380749 | 0.473135 | 0.374602 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCC | | ACTCCCTCG | | | | |
| | 0_1-1_mismatch_A-C | AGUCAUGCAUAGAC | | CGCAACTTT | | | | |
| | 4_1-0_bulge-asymmetric_A- | CACGGCUACAUAAU | | GAAAGTCC | | | | |
| | 6_1-1_mismatch_G-G | UCCUUUUGGAGUCA | | AGTCATGC | | | | |
| | 10_1-1_mismatch_U-C | CUGUCGUCGAAUGG | | ATAGACCA | | | | |
| | 16_2-2_bulge-symmetric_AA-AG | C | | CGGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 192 | −18_6-6_internal_loop- | CUCAGCAGCAGCCA | 631 | CTCAGCAG | 0.518988 | 0.518988 | 0.462196 | 0.368304 |
| | symmetric_UGGUGU-UGGAGU | CAACUCCCUCGCGC | | CAGCCACA | | | | |
| | -6_3-3_bulge-symmetric_CAU-CAU | AACUUUGACAGCCC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GACAGCCC | | | | |
| | 10_1-1_mismatch_U-C | UUCCUUUUGGAGUC | | TTTCATGCA | | | | |
| | 20_1-1_mismatch_A-C | ACUGUCGUCGAAUG | | TAGATCCAC | | | | |
| | 23_1-1_mismatch_U-C | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 193 | -6_6-6_internal_loop- | CUCAGCAGCAGCCA | 632 | CTCAGCAG | 0.371706 | 0.371706 | 0.458365 | 0.3638 |
| | symmetric_AUUCAU-UCAUAC | CAACUCCCUCGCGC | | CAGCCACA | | | | |
| | 0_1-1_mismatch_A-C | AACUGUCCAAGGAA | | ACTCCCTCG | | | | |
| | 10_3-3_bulge-symmetric_UCA-CUU | UUUCACUUAUACAU | | CGCAACTGT | | | | |
| | 18_3-3_bulge-symmetric_GGA-GAA | CCACGGCUAUCAUA | | CCAAGGAA | | | | |
| | 24_4-4_bulge-symmetric_UCAA-GUCC | CUCCUUUACACCAC | | TTTCACTTA | | | | |
| | 30_6-6_internal_loop- | ACUGUCGUCGAAUG | | TACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 194 | -6_6-6_internal_loop- | CUCAGCAGCAGCCA | 633 | CTCAGCAG | 0.375908 | 0.375908 | 0.455997 | 0.375358 |
| | symmetric_AUUCAU-UCAUAC | CAACUCCCUCGCGC | | CAGCCACA | | | | |
| | 0_1-1_mismatch_A-C | AACUUUGAAAGUCA | | ACTCCCTCG | | | | |
| | 7_1-1_mismatch_U-C | UUCAUGAAUCCAUC | | CGCAACTTT | | | | |
| | 17_2-1_bulge-asymmetric_AG-A | CACGGCUAUCAUAC | | GAAAGTCA | | | | |
| | 30_6-6_internal_loop- | UCCUUUACACCACA | | TTCATGAAT | | | | |
| | symmetric_GCCAAG-GCGCAA | CUGUCGUCGAAUGG | | CCATCCACG | | | | |
| | | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 195 | -6_6-6_internal_loop- | CUCAGCAGCAGCCA | 634 | CTCAGCAG | 0.369185 | 0.369185 | 0.458828 | 0.368331 |
| | symmetric_AUUCAU-UCAUAC | CAACUCCCUCGCGC | | CAGCCACA | | | | |
| | 0_1-1_mismatch_A-C | AACUUCCUAAGCGG | | ACTCCCTCG | | | | |
| | 9_3-3_bulge-symmetric_UUC-AGU | GUUCAUAGUUACAU | | CGCAACTTC | | | | |
| | 17_4-4_bulge-symmetric_AGGA-CGGG | CCACGGCUAUCAUA | | CTAAGCGG | | | | |
| | 24_3-3_bulge-symmetric_UCA-CCU | CUCCUUUACACCAC | | GTTCATAGT | | | | |
| | 30_6-6_internal_loop- | ACUGUCGUCGAAUG | | TACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 196 | -6_6-6_internal_loop- | CUCAGCAGCAGCCA | 635 | CTCAGCAG | 0.374213 | 0.374213 | 0.450209 | 0.376236 |
| | symmetric_AUUCAU-UCAUAC | CAACUCCCUCGCGC | | CAGCCACA | | | | |
| | 0_1-1_mismatch_A-C | AACUUUGAAAGUCG | | ACTCCCTCG | | | | |
| | 4_1-0_bulge-asymmetric_A- | UUCAUGAAUGCACC | | CGCAACTTT | | | | |
| | 7_1-1_wobble_U-G | ACGGCUAUCAUACU | | GAAAGTCG | | | | |
| | 17_2-1_bulge-asymmetric_AG-G | CCUUUACACCACAC | | TTCATGAAT | | | | |
| | 30_6-6_internal_loop- | UGUCGUCGAAUGGC | | GCACCACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 197 | -6_6-6_internal_loop- | CUCAGCAGCAGCCA | 636 | CTCAGCAG | 0.376795 | 0.376795 | 0.462887 | 0.370697 |
| | symmetric_AUUCAU-UCAUAC | CAACUCCCUCGCGC | | CAGCCACA | | | | |
| | 0_1-1_mismatch_A-C | AACUUUGAAAGUCA | | ACTCCCTCG | | | | |
| | 6_1-2_bulge-asymmetric_G-AG | UUCAUGAAUAAGAU | | CGCAACTTT | | | | |
| | 17_2-1_bulge-asymmetric_AG-A | CCACGGCUAUCAUA | | GAAAGTCA | | | | |
| | 30_6-6_internal_loop- | CUCCUUUACACCAC | | TTCATGAAT | | | | |
| | symmetric_GCCAAG-GCGCAA | ACUGUCGUCGAAUG | | AAGATCCA | | | | |
| | | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 198 | −18_6-6_internal_loop- | CUCAGCAGCAGCCA | 637 | CTCAGCAG | 0.423226 | 0.423226 | 0.421075 | 0.389519 |
| | symmetric_UGGUGU-UGGAGU | CAACUCCCUCGCGC | | CAGCCACA | | | | |
| | -6_3-3_bulge-symmetric_CAU-CAU | AACUUUGAAAGACC | | ACTCCCTCG | | | | |
| | 0_1-1_mismatch_A-C | CCUUUCAUGCAUAG | | CGCAACTTT | | | | |
| | 4_1-0_bulge-asymmetric_A- | ACCACGGCUACAUA | | GAAAGACC | | | | |
| | 6_1-1_mismatch_G-G | AUUCCUUUUGGAGU | | CCTTTCATG | | | | |
| | 10_1-1_mismatch_U-C | CACUGUCGUCGAAU | | CATAGACC | | | | |
| | 20_1-3_bulge-asymmetric_A-ACC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 199 | -6_6-6_internal_loop- | CUCAGCAGCAGCCA | 638 | CTCAGCAG | 0.371531 | 0.371531 | 0.4419 | 0.366147 |
| | symmetric_AUUCAU-UCAUAC | CAACUCCCUCGCGC | | CAGCCACA | | | | |
| | 0_1-1_mismatch_A-C | AACGACGAAAGGUA | | ACTCCCTCG | | | | |
| | 7_3-3_bulge-symmetric_UAU-UUC | UUUCAUGAUUCCAU | | CGCAACGA | | | | |
| | 18_3-3_bulge-symmetric_GGA-GUA | CCACGGCUAUCAUA | | CGAAAGGT | | | | |
| | 26_3-3_bulge-symmetric_AAA-GAC | CUCCUUUACACCAC | | ATTTCATGA | | | | |
| | 30_6-6_internal_loop- | ACUGUCGUCGAAUG | | TTCCATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 200 | -6_6-6_internal_loop- | CUCAGCAGCAGCCA | 639 | CTCAGCAG | 0.385973 | 0.385973 | 0.427629 | 0.363546 |
| | symmetric_AUUCAU-UCAUAC | CAACUCCCUCGCGC | | CAGCCACA | | | | |
| | 0_1-1_mismatch_A-C | AACUUUGACUGUAU | | ACTCCCTCG | | | | |
| | 10_1-2_bulge-asymmetric_U-CC | UUCAUGCCAUACAU | | CGCAACTTT | | | | |
| | 18_2-1_bulge-asymmetric_GG-A | CCACGGCUAUCAUA | | GACTGTATT | | | | |
| | 22_2-2_bulge-symmetric_UU-CU | CUCCUUUACACCAC | | TCATGCCAT | | | | |
| | 30_6-6_internal_loop- | ACUGUCGUCGAAUG | | ACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 201 | -6_6-6_internal_loop- | CUCAGCAGCAGCCA | 640 | CTCAGCAG | 0.378521 | 0.378521 | 0.417108 | 0.366473 |
| | symmetric_AUUCAU-UCAUAC | CAACUCCCUCGCGC | | CAGCCACA | | | | |
| | 0_1-1_mismatch_A-C | AACUGCUAAAGCGA | | ACTCCCTCG | | | | |
| | 9_3-3_bulge-symmetric_UUC-UGC | UUUCAUUGCUACAU | | CGCAACTG | | | | |
| | 18_3-3_bulge-symmetric_GGA-CGA | CCACGGCUAUCAUA | | CTAAAGCG | | | | |
| | 25_3-3_bulge-symmetric_CAA-GCU | CUCCUUUACACCAC | | ATTTCATTG | | | | |
| | 30_6-6_internal_loop- | ACUGUCGUCGAAUG | | CTACATCCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 202 | −18_6-6_internal_loop- | CUCAGCAGCAGCCA | 641 | CTCAGCAG | 0.375727 | 0.375727 | 0.460237 | 0.389134 |
| | symmetric_UGGUGU-UGGAGU | CAACUCCCUCGCGC | | CAGCCACA | | | | |
| | -6_3-3_bulge-symmetric_CAU-CAU | AACUUUGAAAUGAC | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAATGAC | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 19_3-3_bulge-symmetric_GAC-UGA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 203 | -6_6-6_internal_loop- | CUCAGCAGCAGCCA | 642 | CTCAGCAG | 0.375086 | 0.375086 | 0.447769 | 0.374178 |
| | symmetric_AUUCAU-UCAUAC | CAACUCCCUCGCGC | | CAGCCACA | | | | |
| | 0_1-1_mismatch_A-C | AACUUUGAAAGUAU | | ACTCCCTCG | | | | |
| | 4_1-1_mismatch_A-A | UUCAUGACGACAAC | | CGCAACTTT | | | | |
| | 8_2-2_bulge-symmetric_AU-CG | CACGGCUAUCAUAC | | GAAAGTAT | | | | |
| | 18_2-1_bulge-asymmetric_GG-A | UCCUUUACACCACA | | TTCATGACG | | | | |
| | 30_6-6_internal_loop- | CUGUCGUCGAAUGG | | ACAACCAC | | | | |
| | symmetric_GCCAAG-GCGCAA | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 204 | −18_6-6_internal_loop- | CUCAGCAGCAGCCA | 643 | CTCAGCAG | 0.469091 | 0.469091 | 0.444335 | 0.365124 |
| | symmetric_UGGUGU-UGGAGU | CAACUCCCUCGCGC | | CAGCCACA | | | | |
| | -6_3-3_bulge-symmetric_CAU-CAU | AACUUUGUCUGUCC | | ACTCCCTCG | | | | |
| | 0_1-1_mismatch_A-C | UUUCAUGCAUAGAU | | CGCAACTTT | | | | |
| | 6_1-1_mismatch_G-G | CCACGGCUACAUAA | | GTCTGTCCT | | | | |
| | 10_1-1_mismatch_U-C | UUCCUUUUGGAGUC | | TTCATGCAT | | | | |
| | 22_3-3_bulge-symmetric_UUU-UCU | ACUGUCGUCGAAUG | | AGATCCAC | | | | |
| | 30_6-6_internal_loop- | GC | | GGCTACAT | | | | |
| | symmetric_GCCAAG-GCGCAA | | | AATTCCTTT | | | | |
| | | | | TGGAGTCA | | | | |
| | | | | CTGTCGTCG | | | | |
| | | | | AATGGC | | | | |
| 205 | −18_6-6_internal_loop- | CUCAGCAGCAGCCA | 644 | CTCAGCAG | 0.400768 | 0.400768 | 0.423973 | 0.402003 |
| | symmetric_UGGUGU-UGGAGU | CAACUCCCUCGCGC | | CAGCCACA | | | | |
| | -6_3-3_bulge-symmetric_CAU-CAU | AACUUUGAAACUAC | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAACTAC | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 19_3-3_bulge-symmetric_GAC-CUA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 206 | −18_6-6_internal_loop- | CUCAGCAGCAGCCA | 645 | CTCAGCAG | 0.465496 | 0.465496 | 0.444086 | 0.357103 |
| | symmetric_UGGUGU-UGGAGU | CAACUCCCUCGCGC | | CAGCCACA | | | | |
| | -6_3-3_bulge-symmetric_CAU-CAU | AACUUUGCCAGUCC | | ACTCCCTCG | | | | |
| | 0_1-1_mismatch_A-C | UUUCAUGCAUAGAC | | CGCAACTTT | | | | |
| | 4_1-1_mismatch_A-C | CCACGGCUACAUAA | | GCCAGTCCT | | | | |
| | 6_1-1_mismatch_G-G | UUCCUUUUGGAGUC | | TTCATGCAT | | | | |
| | 10_1-1_mismatch_U-C | ACUGUCGUCGAAUG | | AGACCCAC | | | | |
| | 23_2-2_bulge-symmetric_UU-CC | GC | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 207 | -8_6-6_internal_loop- | CUCAGCAGCAGCCA | 646 | CTCAGCAG | 0.36974 | 0.36974 | 0.47951 | 0.416564 |
| | symmetric_GAAUUC-CGCCGA | CAACUCCCUCGCGC | | CAGCCACA | | | | |
| | -4_1-0_bulge-asymmetric_A- | AACUUUGUUUCCUU | | ACTCCCTCG | | | | |
| | 0_1-1_mismatch_A-C | UCAUGUGCCCAUCC | | CGCAACTTT | | | | |
| | 7_4-4_bulge-symmetric_UAUU-UGCC | ACGGCAAUCGCCGA | | GTTTCCTTT | | | | |
| | 21_4-2_bulge-asymmetric_CUUU-UU | CUUUACACCACACU | | CATGTGCCC | | | | |
| | 30_6-6_internal_loop- | GUCGUCGAAUGGC | | ATCCACGG | | | | |
| | symmetric_GCCAAG-GCGCAA | | | CAATCGCC | | | | |
| | | | | GACTTTACA | | | | |
| | | | | CCACACTGT | | | | |
| | | | | CGTCGAAT | | | | |
| | | | | GGC | | | | |
| 208 | −18_6-6_internal_loop- | CUCAGCAGCAGCCA | 647 | CTCAGCAG | 0.413117 | 0.413117 | 0.419993 | 0.396682 |
| | symmetric_UGGUGU-UGGAGU | CAACUCCCUCGCGC | | CAGCCACA | | | | |
| | -6_3-3_bulge-symmetric_CAU-CAU | AACUUUGAAAACUU | | ACTCCCTCG | | | | |
| | 0_1-1_mismatch_A-C | UCAUGCAUAGAUCG | | CGCAACTTT | | | | |
| | 2_1-1_mismatch_G-G | ACGGCUACAUAAUU | | GAAAACTTT | | | | |
| | 6_1-1_mismatch_G-G | CCUUUUGGAGUCAC | | CATGCATA | | | | |
| | 10_1-1_mismatch_U-C | UGUCGUCGAAUGGC | | GATCGACG | | | | |
| | 19_3-1_bulge-asymmetric_GAC-A | | | GCTACATA | | | | |
| | 30_6-6_internal_loop- | | | ATTCCTTTT | | | | |
| | symmetric_GCCAAG-GCGCAA | | | GGAGTCAC | | | | |
| | | | | TGTCGTCGA | | | | |
| | | | | ATGGC | | | | |
| 209 | -6_6-6_internal_loop- | CUCAGCAGCAGCCA | 648 | CTCAGCAG | 0.383241 | 0.383241 | 0.425916 | 0.370338 |
| | symmetric_AUUCAU-UCAUAC | CAACUCCCUCGCGC | | CAGCCACA | | | | |
| | 0_1-1_mismatch_A-C | AACUUUGAAAGUCA | | ACTCCCTCG | | | | |
| | 9_1-1_mismatch_U-C | UUCAUGACUACAUC | | CGCAACTTT | | | | |
| | 17_2-1_bulge-asymmetric_AG-A | CACGGCUAUCAUAC | | GAAAGTCA | | | | |
| | 30_6-6_internal_loop- | UCCUUUACACCACA | | TTCATGACT | | | | |
| | symmetric_GCCAAG-GCGCAA | CUGUCGUCGAAUGG | | ACATCCAC | | | | |
| | | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 210 | -6_6-6_internal_loop- | CUCAGCAGCAGCCA | 649 | CTCAGCAG | 0.370414 | 0.370414 | 0.443955 | 0.376148 |
| | symmetric_AUUCAU-UCAUAC | CAACUCCCUCGCGC | | CAGCCACA | | | | |
| | 0_1-1_mismatch_A-C | AACUUCAAGUAAGC | | ACTCCCTCG | | | | |
| | 10_1-1_mismatch_U-C | UUUCAUGCAUACAU | | CGCAACTTC | | | | |
| | 19_1-3_bulge-asymmetric_G-AAG | CCACGGCUAUCAUA | | AAGTAAGC | | | | |
| | 24_3-1_bulge-asymmetric_UCA-C | CUCCUUUACACCAC | | TTTCATGCA | | | | |
| | 30_6-6_internal_loop- | ACUGUCGUCGAAUG | | TACATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 211 | -8_6-6_internal_loop- | CUCAGCAGCAGCCA | 650 | CTCAGCAG | 0.393743 | 0.393743 | 0.427227 | 0.394587 |
| | symmetric_GAAUUC-CGCCGA | CAACUCCCUCGCGC | | CAGCCACA | | | | |
| | -4_1-0_bulge-asymmetric_A- | AACUUUGACCACCU | | ACTCCCTCG | | | | |
| | 0_1-1_mismatch_A-C | UUCAUGCUACACAU | | CGCAACTTT | | | | |
| | 8_3-4_bulge-asymmetric_AUU-CUAC | CCACGGCAAUCGCC | | GACCACCTT | | | | |
| | 20_4-3_bulge-asymmetric_ACUU-CCA | GACUUUACACCACA | | TCATGCTAC | | | | |
| | 30_6-6_internal_loop- | CUGUCGUCGAAUGG | | ACATCCAC | | | | |
| | symmetric_GCCAAG-GCGCAA | C | | GGCAATCG | | | | |
| | | | | CCGACTTTA | | | | |
| | | | | CACCACACT | | | | |
| | | | | GTCGTCGA | | | | |
| | | | | ATGGC | | | | |
| 212 | −18_6-6_internal_loop- | CUCAGCAGCAGCCA | 651 | CTCAGCAG | 0.367888 | 0.367888 | 0.44981 | 0.398963 |
| | symmetric_UGGUGU-UGGAGU | CAACUCCCUCGCGC | | CAGCCACA | | | | |
| | -6_3-3_bulge-symmetric_CAU-CAU | AACUUUGAAAGUAA | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGTAA | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_2-2_bulge-symmetric_GG-AA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 213 | -6_6-6_internal_loop- | CUCAGCAGCAGCCA | 652 | CTCAGCAG | 0.367379 | 0.367379 | 0.465023 | 0.382499 |
| | symmetric_AUUCAU-UCAUAC | CAACUCCCUCGCGC | | CAGCCACA | | | | |
| | 0_1-1_mismatch_A-C | AACUCAUGAUGUCC | | ACTCCCTCG | | | | |
| | 2_1-1_wobble_G-U | UUUCAUGAAUACAU | | CGCAACTC | | | | |
| | 22_2-1_bulge-asymmetric_UU-U | CUACGGCUAUCAUA | | ATGATGTCC | | | | |
| | 27_1-2_bulge-asymmetric_A-CA | CUCCUUUACACCAC | | TTTCATGAA | | | | |
| | 30_6-6_internal_loop- | ACUGUCGUCGAAUG | | TACATCTAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 214 | -6_6-6_internal_loop- | CUCAGCAGCAGCCA | 653 | CTCAGCAG | 0.374401 | 0.374401 | 0.447292 | 0.376381 |
| | symmetric_AUUCAU-UCAUAC | CAACUCCCUCGCGC | | CAGCCACA | | | | |
| | 0_1-1_mismatch_A-C | AACUUUGAAAGUCG | | ACTCCCTCG | | | | |
| | 7_2-1_bulge-asymmetric_UA-C | UUCAUGAACCAUCC | | CGCAACTTT | | | | |
| | 17_2-1_bulge-asymmetric_AG-G | ACGGCUAUCAUACU | | GAAAGTCG | | | | |
| | 30_6-6_internal_loop- | CCUUUACACCACAC | | TTCATGAAC | | | | |
| | symmetric_GCCAAG-GCGCAA | UGUCGUCGAAUGGC | | CATCCACG | | | | |
| | | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 215 | -6_6-6_internal_loop- | CUCAGCAGCAGCCA | 654 | CTCAGCAG | 0.37072 | 0.37072 | 0.446201 | 0.373084 |
| | symmetric_AUUCAU-UCAUAC | CAACUCCCUCGCGC | | CAGCCACA | | | | |
| | 0_1-1_mismatch_A-C | AACUCUAAAAGUGG | | ACTCCCTCG | | | | |
| | 7_4-4_bulge-symmetric_UAUU-CGGC | GUUCAUGCGGCCAU | | CGCAACTCT | | | | |
| | 17_3-3_bulge-symmetric_AGG-GGG | CCACGGCUAUCAUA | | AAAAGTGG | | | | |
| | 25_3-3_bulge-symmetric_CAA-CUA | CUCCUUUACACCAC | | GTTCATGCG | | | | |
| | 30_6-6_internal_loop- | ACUGUCGUCGAAUG | | GCCATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 216 | -8_6-6_internal_loop- | CUCAGCAGCAGCCA | 655 | CTCAGCAG | 0.380197 | 0.380197 | 0.444423 | 0.421221 |
| | symmetric_GAAUUC-CGCCGA | CAACUCCCUCGCGC | | CAGCCACA | | | | |
| | -4_1-0_bulge-asymmetric_A- | AACUUUGAACGCUU | | ACTCCCTCG | | | | |
| | 0_1-1_mismatch_A-C | UCCUCCAUACAUCC | | CGCAACTTT | | | | |
| | 10_4-4_bulge-symmetric_UCAU-CUCC | ACGGCAAUCGCCGA | | GAACGCTTT | | | | |
| | 19_4-2_bulge-asymmetric_GACU-CG | CUUUACACCACACU | | CCTCCATAC | | | | |
| | 30_6-6_internal_loop- | GUCGUCGAAUGGC | | ATCCACGG | | | | |
| | symmetric_GCCAAG-GCGCAA | | | CAATCGCC | | | | |
| | | | | GACTTTACA | | | | |
| | | | | CCACACTGT | | | | |
| | | | | CGTCGAAT | | | | |
| | | | | GGC | | | | |
| 217 | -6_6-6_internal_loop- | CUCAGCAGCAGCCA | 656 | CTCAGCAG | 0.364904 | 0.364904 | 0.463954 | 0.368628 |
| | symmetric_AUUCAU-UCAUAC | CAACUCCCUCGCGC | | CAGCCACA | | | | |
| | 0_1-1_mismatch_A-C | AACUUCCCAAGAUU | | ACTCCCTCG | | | | |
| | 9_3-3_bulge-symmetric_UUC-CCU | CAUCCUUACAUCCA | | CGCAACTTC | | | | |
| | 17_4-1_bulge-asymmetric_AGGA-A | CGGCUAUCAUACUC | | CCAAGATTC | | | | |
| | 24_3-3_bulge-symmetric_UCA-CCC | CUUUACACCACACU | | ATCCTTACA | | | | |
| | 30_6-6_internal_loop- | GUCGUCGAAUGGC | | TCCACGGCT | | | | |
| | symmetric_GCCAAG-GCGCAA | | | ATCATACTC | | | | |
| | | | | CTTTACACC | | | | |
| | | | | ACACTGTCG | | | | |
| | | | | TCGAATGG | | | | |
| | | | | C | | | | |
| 218 | −18_6-6_internal_loop- | CUCAGCAGCAGCCA | 657 | CTCAGCAG | 0.466849 | 0.466849 | 0.464008 | 0.352477 |
| | symmetric_UGGUGU-UGGAGU | CAACUCCCUCGCGC | | CAGCCACA | | | | |
| | -6_3-3_bulge-symmetric_CAU-CAU | AACUUUGUCAAGUA | | ACTCCCTCG | | | | |
| | 0_1-1_mismatch_A-C | CUUUCAUGCAUAGA | | CGCAACTTT | | | | |
| | 6_1-1_mismatch_G-G | UCCACGGCUACAUA | | GTCAAGTA | | | | |
| | 10_1-1_mismatch_U-C | AUUCCUUUUGGAGU | | CTTTCATGC | | | | |
| | 19_1-1_mismatch_G-A | CACUGUCGUCGAAU | | ATAGATCC | | | | |
| | 24_1-2_bulge-asymmetric_U-UC | GGC | | ACGGCTAC | | | | |
| | 30_6-6_internal_loop- | | | ATAATTCCT | | | | |
| | symmetric_GCCAAG-GCGCAA | | | TTTGGAGTC | | | | |
| | | | | ACTGTCGTC | | | | |
| | | | | GAATGGC | | | | |
| 219 | −18_6-6_internal_loop- | CUCAGCAGCAGCCA | 658 | CTCAGCAG | 0.393566 | 0.393566 | 0.413055 | 0.360877 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCC | | ACTCCCTCG | | | | |
| | 0_1-1_mismatch_A-C | AGGCAUGCAUAGAC | | CGCAACTTT | | | | |
| | 4_1-0_bulge-asymmetric_A- | CACGGCUACAUAAU | | GAAAGTCC | | | | |
| | 6_1-1_mismatch_G-G | UCCUUUUGGAGUCA | | AGGCATGC | | | | |
| | 10_1-1_mismatch_U-C | CUGUCGUCGAAUGG | | ATAGACCA | | | | |
| | 15_3-3_bulge-symmetric_AAA-AGG | C | | CGGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 220 | −18_6-6_internal_loop- | CUCAGCAGCAGCCA | 659 | CTCAGCAG | 0.3669 | 0.3669 | 0.467411 | 0.393366 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGA | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGTGA | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_2-2_bulge-symmetric_GG-GA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 221 | -6_6-6_internal_loop- | CUCAGCAGCAGCCA | 660 | CTCAGCAG | 0.37407 | 0.37407 | 0.443725 | 0.382628 |
| | symmetric_AUUCAU-UCAUAC | CAACUCCCUCGCGC | | CAGCCACA | | | | |
| | 0_1-1_mismatch_A-C | AACUUUGAAAGAUU | | ACTCCCTCG | | | | |
| | 5_1-1_mismatch_U-U | UCAUGACUUACUUC | | CGCAACTTT | | | | |
| | 9_1-2_bulge-asymmetric_U-CU | CACGGCUAUCAUAC | | GAAAGATT | | | | |
| | 18_3-1_bulge-asymmetric_GGA-A | UCCUUUACACCACA | | TCATGACTT | | | | |
| | 30_6-6_internal_loop- | CUGUCGUCGAAUGG | | ACTTCCACG | | | | |
| | symmetric_GCCAAG-GCGCAA | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 222 | -6_6-6_internal_loop- | CUCAGCAGCAGCCA | 661 | CTCAGCAG | 0.367037 | 0.367037 | 0.433614 | 0.364926 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 4_1-0_bulge-asymmetric_A- | UUCAUGAAACCACC | | CGCAACTTT | | | | |
| | 7_2-2_bulge-symmetric_UA-AC | ACGGCUAUCAUACU | | GAAAGTGT | | | | |
| | 18_2-1_bulge-asymmetric_GG-G | CCUUUACACCACAC | | TTCATGAAA | | | | |
| | 30_6-6_internal_loop- | UGUCGUCGAAUGGC | | CCACCACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 223 | -6_6-6_internal_loop- | CUCAGCAGCAGCCA | 662 | CTCAGCAG | 0.361592 | 0.361592 | 0.468941 | 0.38063 |
| | symmetric_AUUCAU-UCAUAC | CAACUCCCUCGCGC | | CAGCCACA | | | | |
| | 0_1-1_mismatch_A-C | AACUUUCUUAGUAG | | ACTCCCTCG | | | | |
| | 5_3-3_bulge-symmetric_UGU-CUC | GUUCAUGAAUCUCU | | CGCAACTTT | | | | |
| | 17_3-3_bulge-symmetric_AGG-AGG | CCACGGCUAUCAUA | | CTTAGTAGG | | | | |
| | 23_3-3_bulge-symmetric_UUC-CUU | CUCCUUUACACCAC | | TTCATGAAT | | | | |
| | 30_6-6_internal_loop- | ACUGUCGUCGAAUG | | CTCTCCACG | | | | |
| | symmetric_GCCAAG-GCGCAA | GC | | GCTATCATA | | | | |
| | | | | CTCCTTTAC | | | | |
| | | | | ACCACACT | | | | |
| | | | | GTCGTCGA | | | | |
| | | | | ATGGC | | | | |
| 224 | -6_6-6_internal_loop- | CUCAGCAGCAGCCA | 663 | CTCAGCAG | 0.375356 | 0.375356 | 0.410516 | 0.356876 |
| | symmetric_AUUCAU-UCAUAC | CAACUCCCUCGCGC | | CAGCCACA | | | | |
| | 0_1-1_mismatch_A-C | AACUUUGAAUUGCG | | ACTCCCTCG | | | | |
| | 9_3-3_bulge-symmetric_UUC-UGU | UGGGUUCAUUGUUA | | CGCAACTTT | | | | |
| | 17_3-3_bulge-symmetric_AGG-GGG | CAUCCACGGCUAUC | | GAATTGCGT | | | | |
| | 22_1-4_bulge-asymmetric_U-UUGC | AUACUCCUUUACAC | | GGGTTCATT | | | | |
| | 30_6-6_internal_loop- | CACACUGUCGUCGA | | GTTACATCC | | | | |
| | symmetric_GCCAAG-GCGCAA | AUGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 225 | -6_6-6_internal_loop- | CUCAGCAGCAGCCA | 664 | CTCAGCAG | 0.372655 | 0.372655 | 0.434771 | 0.386047 |
| | symmetric_AUUCAU-UCAUAC | CAACUCCCUCGCGC | | CAGCCACA | | | | |
| | 0_1-1_mismatch_A-C | AACUUUGACUGUCG | | ACTCCCTCG | | | | |
| | 7_1-1_mismatch_U-C | UUCAUGAAUCCAUC | | CGCAACTTT | | | | |
| | 17_2-1_bulge-asymmetric_AG-G | CACGGCUAUCAUAC | | GACTGTCGT | | | | |
| | 22_2-2_bulge-symmetric_UU-CU | UCCUUUACACCACA | | TCATGAATC | | | | |
| | 30_6-6_internal_loop- | CUGUCGUCGAAUGG | | CATCCACG | | | | |
| | symmetric_GCCAAG-GCGCAA | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 226 | -6_6-6_internal_loop- | CUCAGCAGCAGCCA | 665 | CTCAGCAG | 0.36604 | 0.36604 | 0.450439 | 0.373056 |
| | symmetric_AUUCAU-UCAUAC | CAACUCCCUCGCGC | | CAGCCACA | | | | |
| | 0_1-1_mismatch_A-C | AACUGUUAAAGUCC | | ACTCCCTCG | | | | |
| | 2_1-1_wobble_G-U | UUUCACUCAUACAU | | CGCAACTGT | | | | |
| | 10_3-3_bulge-symmetric_UCA-CUC | CUACGGCUAUCAUA | | TAAAGTCCT | | | | |
| | 25_3-3_bulge-symmetric_CAA-GUU | CUCCUUUACACCAC | | TTCACTCAT | | | | |
| | 30_6-6_internal_loop- | ACUGUCGUCGAAUG | | ACATCTACG | | | | |
| | symmetric_GCCAAG-GCGCAA | GC | | GCTATCATA | | | | |
| | | | | CTCCTTTAC | | | | |
| | | | | ACCACACT | | | | |
| | | | | GTCGTCGA | | | | |
| | | | | ATGGC | | | | |
| 227 | -6_6-6_internal_loop- | CUCAGCAGCAGCCA | 666 | CTCAGCAG | 0.360414 | 0.360414 | 0.454877 | 0.398172 |
| | symmetric_AUUCAU-UCAUAC | CAACUCCCUCGCGC | | CAGCCACA | | | | |
| | 0_1-1_mismatch_A-C | AACGUCGAUUACUC | | ACTCCCTCG | | | | |
| | 4_3-3_bulge-symmetric_AUG-AAG | CUUUCAUGAAUAAA | | CGCAACGT | | | | |
| | 21_3-4_bulge-asymmetric_CUU-UUAC | GCCACGGCUAUCAU | | CGATTACTC | | | | |
| | 26_3-3_bulge-symmetric_AAA-GUC | ACUCCUUUACACCA | | CTTTCATGA | | | | |
| | 30_6-6_internal_loop- | CACUGUCGUCGAAU | | ATAAAGCC | | | | |
| | symmetric_GCCAAG-GCGCAA | GGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 228 | -6_6-6_internal_loop- | CUCAGCAGCAGCCA | 667 | CTCAGCAG | 0.36671 | 0.36671 | 0.441126 | 0.353259 |
| | symmetric_AUUCAU-UCAUAC | CAACUCCCUCGCGC | | CAGCCACA | | | | |
| | 0_1-1_mismatch_A-C | AACUUUGAAAGGGC | | ACTCCCTCG | | | | |
| | 6_2-2_bulge-symmetric_GU-CG | UUUCAUGAAUCGAU | | CGCAACTTT | | | | |
| | 19_2-2_bulge-symmetric_GA-GG | CCACGGCUAUCAUA | | GAAAGGGC | | | | |
| | 30_6-6_internal_loop- | CUCCUUUACACCAC | | TTTCATGAA | | | | |
| | symmetric_GCCAAG-GCGCAA | ACUGUCGUCGAAUG | | TCGATCCAC | | | | |
| | | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 229 | -6_6-6_internal_loop- | CUCAGCAGCAGCCA | 668 | CTCAGCAG | 0.373567 | 0.373567 | 0.432598 | 0.363719 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 7_2-1_bulge-asymmetric_UA-C | UUCAUGAACCAUCC | | CGCAACTTT | | | | |
| | 18_2-1_bulge-asymmetric_GG-G | ACGGCUAUCAUACU | | GAAAGTGT | | | | |
| | 30_6-6_internal_loop- | CCUUUACACCACAC | | TTCATGAAC | | | | |
| | symmetric_GCCAAG-GCGCAA | UGUCGUCGAAUGGC | | CATCCACG | | | | |
| | | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 230 | -6_6-6_internal_loop- | CUCAGCAGCAGCCA | 669 | CTCAGCAG | 0.362587 | 0.362587 | 0.444329 | 0.363385 |
| | symmetric_AUUCAU-UCAUAC | CAACUCCCUCGCGC | | CAGCCACA | | | | |
| | 0_1-1_mismatch_A-C | AACGAAGAACAACC | | ACTCCCTCG | | | | |
| | 9_2-4_bulge-asymmetric_UU-UGUU | UUUCAUGUGUUUAC | | CGCAACGA | | | | |
| | 20_3-3_bulge-symmetric_ACU-CAA | AUCCACGGCUAUCA | | AGAACAAC | | | | |
| | 26_3-3_bulge-symmetric_AAA-GAA | UACUCCUUUACACC | | CTTTCATGT | | | | |
| | 30_6-6_internal_loop- | ACACUGUCGUCGAA | | GTTTACATC | | | | |
| | symmetric_GCCAAG-GCGCAA | UGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 231 | -6_6-6_internal_loop- | CUCAGCAGCAGCCA | 670 | CTCAGCAG | 0.371599 | 0.371599 | 0.435016 | 0.370124 |
| | symmetric_AUUCAU-UCAUAC | CAACUCCCUCGCGC | | CAGCCACA | | | | |
| | 0_1-1_mismatch_A-C | AACUUUGUUAAGAC | | ACTCCCTCG | | | | |
| | 5_1-2_bulge-asymmetric_U-UC | UUUCAUGAAUACUC | | CGCAACTTT | | | | |
| | 19_2-1_bulge-asymmetric_GA-A | UCCACGGCUAUCAU | | GTTAAGACT | | | | |
| | 24_1-2_bulge-asymmetric_U-UU | ACUCCUUUACACCA | | TTCATGAAT | | | | |
| | 30_6-6_internal_loop- | CACUGUCGUCGAAU | | ACTCTCCAC | | | | |
| | symmetric_GCCAAG-GCGCAA | GGC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 232 | -6_6-6_internal_loop- | CUCAGCAGCAGCCA | 671 | CTCAGCAG | 0.374705 | 0.374705 | 0.44149 | 0.359745 |
| | symmetric_AUUCAU-UCAUAC | CAACUCCCUCGCGC | | CAGCCACA | | | | |
| | 0_1-1_mismatch_A-C | AACUUUGAUCGUCC | | ACTCCCTCG | | | | |
| | 7_1-1_mismatch_U-C | UUUGAUGAAUCCAU | | CGCAACTTT | | | | |
| | 14_1-1_mismatch_G-G | CCACGGCUAUCAUA | | GATCGTCCT | | | | |
| | 22_2-2_bulge-symmetric_UU-UC | CUCCUUUACACCAC | | TTGATGAAT | | | | |
| | 30_6-6_internal_loop- | ACUGUCGUCGAAUG | | CCATCCACG | | | | |
| | symmetric_GCCAAG-GCGCAA | GC | | GCTATCATA | | | | |
| | | | | CTCCTTTAC | | | | |
| | | | | ACCACACT | | | | |
| | | | | GTCGTCGA | | | | |
| | | | | ATGGC | | | | |
| 233 | -6_6-6_internal_loop- | CUCAGCAGCAGCCA | 672 | CTCAGCAG | 0.358325 | 0.358325 | 0.469112 | 0.376877 |
| | symmetric_AUUCAU-UCAUAC | CAACUCCCUCGCGC | | CAGCCACA | | | | |
| | 0_1-1_mismatch_A-C | AACUUUUAGUAUUU | | ACTCCCTCG | | | | |
| | 4_1-1_mismatch_A-A | CAUGAAUACAACCA | | CGCAACTTT | | | | |
| | 18_2-1_bulge-asymmetric_GG-A | CGGCUAUCAUACUC | | TAGTATTTC | | | | |
| | 23_3-1_bulge-asymmetric_UUC-U | CUUUACACCACACU | | ATGAATAC | | | | |
| | 30_6-6_internal_loop- | GUCGUCGAAUGGC | | AACCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 234 | -6_6-6_internal_loop- | CUCAGCAGCAGCCA | 673 | CTCAGCAG | 0.367951 | 0.367951 | 0.445542 | 0.348794 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 5_1-2_bulge-asymmetric_U-UU | UUCAUGAAUACUUU | | CGCAACTTT | | | | |
| | 18_2-1_bulge-asymmetric_GG-G | CCACGGCUAUCAUA | | GAAAGTGT | | | | |
| | 30_6-6_internal_loop- | CUCCUUUACACCAC | | TTCATGAAT | | | | |
| | symmetric_GCCAAG-GCGCAA | ACUGUCGUCGAAUG | | ACTTTCCAC | | | | |
| | | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 235 | -6_6-6_internal_loop- | CUCAGCAGCAGCCA | 674 | CTCAGCAG | 0.364691 | 0.364691 | 0.43834 | 0.391614 |
| | symmetric_AUUCAU-UCAUAC | CAACUCCCUCGCGC | | CAGCCACA | | | | |
| | 0_1-1_mismatch_A-C | AACUUUGAAAGACU | | ACTCCCTCG | | | | |
| | 7_1-1_mismatch_U-C | UUCAUGAAUCCAUC | | CGCAACTTT | | | | |
| | 19_2-1_bulge-asymmetric_GA-A | CACGGCUAUCAUAC | | GAAAGACT | | | | |
| | 30_6-6_internal_loop- | UCCUUUACACCACA | | TTCATGAAT | | | | |
| | symmetric_GCCAAG-GCGCAA | CUGUCGUCGAAUGG | | CCATCCACG | | | | |
| | | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 236 | -6_6-6_internal_loop- | CUCAGCAGCAGCCA | 675 | CTCAGCAG | 0.360139 | 0.360139 | 0.432415 | 0.352929 |
| | symmetric_AUUCAU-UCAUAC | CAACUCCCUCGCGC | | CAGCCACA | | | | |
| | 0_1-1_mismatch_A-C | AACUUCCUAAGGGU | | ACTCCCTCG | | | | |
| | 9_3-3_bulge-symmetric_UUC-CCU | UCAUCCUUACAUCC | | CGCAACTTC | | | | |
| | 17_4-2_bulge-asymmetric_AGGA-GG | ACGGCUAUCAUACU | | CTAAGGGTT | | | | |
| | 24_3-3_bulge-symmetric_UCA-CCU | CCUUUACACCACAC | | CATCCTTAC | | | | |
| | 30_6-6_internal_loop- | UGUCGUCGAAUGGC | | ATCCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 237 | −18_6-6_internal_loop- | CUCAGCAGCAGCCA | 676 | CTCAGCAG | 0.366015 | 0.366015 | 0.478145 | 0.372533 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCC | | ACTCCCTCG | | | | |
| | 0_1-1_mismatch_A-C | GUUCAUGCAUAGAG | | CGCAACTTT | | | | |
| | 4_1-1_mismatch_A-G | CCACGGCUACAUAA | | GAAAGTCC | | | | |
| | 6_1-1_mismatch_G-G | UUCCUUUUGGAGUC | | GTTCATGCA | | | | |
| | 10_1-1_mismatch_U-C | | | TAGAGCCA | | | | |
| | 17_1-1_mismatch_A-G | ACUGUCGUCGAAUG | | CGGCTACAT | | | | |
| | 30_6-6_internal_loop- | GC | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 238 | -6_6-6_internal_loop- | CUCAGCAGCAGCCA | 677 | CTCAGCAG | 0.360344 | 0.360344 | 0.443107 | 0.353927 |
| | symmetric_AUUCAU-UCAUAC | CAACUCCCUCGCGC | | CAGCCACA | | | | |
| | 0_1-1_mismatch_A-C | AACAUUGAAAGUCC | | ACTCCCTCG | | | | |
| | 3_1-1_wobble_G-U | UUUCAUGAAUAGUU | | CGCAACATT | | | | |
| | 5_2-2_bulge-symmetric_UG-GU | UCACGGCUAUCAUA | | GAAAGTCC | | | | |
| | 28_1-1_mismatch_A-A | CUCCUUUACACCAC | | TTTCATGAA | | | | |
| | 30_6-6_internal_loop- | ACUGUCGUCGAAUG | | TAGTTTCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 239 | -6_6-6_internal_loop- | CUCAGCAGCAGCCA | 678 | CTCAGCAG | 0.355378 | 0.355378 | 0.450198 | 0.375463 |
| | symmetric_AUUCAU-UCAUAC | CAACUCCCUCGCGC | | CAGCCACA | | | | |
| | 0_1-1_mismatch_A-C | AACUUAUAACCUCC | | ACTCCCTCG | | | | |
| | 6_1-1_mismatch_G-G | UUUCAUGAAUAGAU | | CGCAACTTA | | | | |
| | 21_2-2_bulge-symmetric_CU-CC | CCACGGCUAUCAUA | | TAACCTCCT | | | | |
| | 25_2-2_bulge-symmetric_CA-AU | CUCCUUUACACCAC | | TTCATGAAT | | | | |
| | 30_6-6_internal_loop- | ACUGUCGUCGAAUG | | AGATCCAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 240 | -6_6-6_internal_loop- | CUCAGCAGCAGCCA | 679 | CTCAGCAG | 0.385065 | 0.385065 | 0.43052 | 0.35437 |
| | symmetric_AUUCAU-UCAUAC | CAACUCCCUCGCGC | | CAGCCACA | | | | |
| | 0_1-1_mismatch_A-C | AACUUUGAUCGUCC | | ACTCCCTCG | | | | |
| | 2_1-1_wobble_G-U | UUUCAUCUACAUCU | | CGCAACTTT | | | | |
| | 9_3-1_bulge-asymmetric_UUC-C | ACGGCUAUCAUACU | | GATCGTCCT | | | | |
| | 22_2-2_bulge-symmetric_UU-UC | CCUUUACACCACAC | | TTCATCTAC | | | | |
| | 30_6-6_internal_loop- | UGUCGUCGAAUGGC | | ATCTACGGC | | | | |
| | symmetric_GCCAAG-GCGCAA | | | TATCATACT | | | | |
| | | | | CCTTTACAC | | | | |
| | | | | CACACTGTC | | | | |
| | | | | GTCGAATG | | | | |
| | | | | GC | | | | |
| 241 | -8_6-6_internal_loop- | CUCAGCAGCAGCCA | 680 | CTCAGCAG | 0.357401 | 0.357401 | 0.46409 | 0.40595 |
| | symmetric_GAAUUC-CGCCGA | CAACUCCCUCGCGC | | CAGCCACA | | | | |
| | -4_1-0_bulge-asymmetric_A- | AACUUUGACCCUUU | | ACTCCCTCG | | | | |
| | 0_1-1_mismatch_A-C | CAUGUCUUUACAUC | | CGCAACTTT | | | | |
| | 9_2-4_bulge-asymmetric_UU-UCUU | CACGGCAAUCGCCG | | GACCCTTTC | | | | |
| | 20_4-1_bulge-asymmetric_ACUU-C | ACUUUACACCACAC | | ATGTCTTTA | | | | |
| | 30_6-6_internal_loop- | UGUCGUCGAAUGGC | | CATCCACG | | | | |
| | symmetric_GCCAAG-GCGCAA | | | GCAATCGC | | | | |
| | | | | CGACTTTAC | | | | |
| | | | | ACCACACT | | | | |
| | | | | GTCGTCGA | | | | |
| | | | | ATGGC | | | | |
| 242 | −18_6-6_internal_loop- | CUCAGCAGCAGCCA | 681 | CTCAGCAG | 0.366846 | 0.366846 | 0.427511 | 0.377803 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCA | | ACTCCCTCG | | | | |
| | 0_1-1_mismatch_A-C | UCUCAUGCAUAGAC | | CGCAACTTT | | | | |
| | 4_1-0_bulge-asymmetric_A- | CACGGCUACAUAAU | | GAAAGTCA | | | | |
| | 6_1-1_mismatch_G-G | UCCUUUUGGAGUCA | | TCTCATGCA | | | | |
| | 10_1-1_mismatch_U-C | CUGUCGUCGAAUGG | | TAGACCAC | | | | |
| | 16_3-3_bulge-symmetric_AAG-AUC | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 243 | -6_6-6_internal_loop- | CUCAGCAGCAGCCA | 682 | CTCAGCAG | 0.358979 | 0.358979 | 0.445669 | 0.360657 |
| | symmetric_AUUCAU-UCAUAC | CAACUCCCUCGCGC | | CAGCCACA | | | | |
| | 0_1-1_mismatch_A-C | AACGAUGAUGCAGU | | ACTCCCTCG | | | | |
| | 6_1-3_bulge-asymmetric_G-AAG | CCUUUCAUGAAUAA | | CGCAACGA | | | | |
| | 23_1-3_bulge-asymmetric_U-UGC | AGAUCCACGGCUAU | | TGATGCAGT | | | | |
| | 27_2-2_bulge-symmetric_AA-GA | CAUACUCCUUUACA | | CCTTTCATG | | | | |
| | 30_6-6_internal_loop- | CCACACUGUCGUCG | | AATAAAGA | | | | |
| | symmetric_GCCAAG-GCGCAA | AAUGGC | | TCCACGGCT | | | | |
| | | | | ATCATACTC | | | | |
| | | | | CTTTACACC | | | | |
| | | | | ACACTGTCG | | | | |
| | | | | TCGAATGG | | | | |
| | | | | C | | | | |
| 244 | -10_6-6_internal_loop- | CUCAGCAGCAGCCA | 683 | CTCAGCAG | 0.374722 | 0.374722 | 0.455272 | 0.402004 |
| | symmetric_AGGAAU-UAGAGC | CAACUCCCUCCUCU | | CAGCCACA | | | | |
| | -6_2-2_bulge-symmetric_AU-CG | CGAGUUGAAAGAGC | | ACTCCCTCC | | | | |
| | 0_1-1_mismatch_A-C | UUUCAUGAAUACAA | | TCTCGAGTT | | | | |
| | 4_1-1_mismatch_A-A | CCACGGCUACGGAU | | GAAAGAGC | | | | |
| | 19_2-2_bulge-symmetric_GA-AG | AGAGCUUACACCAC | | TTTCATGAA | | | | |
| | 28_6-6_internal_loop- | ACUGUCGUCGAAUG | | TACAACCA | | | | |
| | symmetric_AGGCCA-CUCGAG | GC | | CGGCTACG | | | | |
| | | | | GATAGAGC | | | | |
| | | | | TTACACCAC | | | | |
| | | | | ACTGTCGTC | | | | |
| | | | | GAATGGC | | | | |
| 245 | -6_6-6_internal_loop- | CUCAGCAGCAGCCA | 684 | CTCAGCAG | 0.352358 | 0.352358 | 0.456377 | 0.383674 |
| | symmetric_AUUCAU-UCAUAC | CAACUCCCUCGCGC | | CAGCCACA | | | | |
| | 0_1-1_mismatch_A-C | AACUUUGAUGACGU | | ACTCCCTCG | | | | |
| | 9_3-3_bulge-symmetric_UUC-CUU | GGGUUCAUCUUUAC | | CGCAACTTT | | | | |
| | 17_3-3_bulge-symmetric_AGG-GGG | AUCCACGGCUAUCA | | GATGACGT | | | | |
| | 22_2-4_bulge-asymmetric_UU-UGAC | UACUCCUUUACACC | | GGGTTCATC | | | | |
| | 30_6-6_internal_loop- | ACACUGUCGUCGAA | | TTTACATCC | | | | |
| | symmetric_GCCAAG-GCGCAA | UGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 246 | -6_6-6_internal_loop- | CUCAGCAGCAGCCA | 685 | CTCAGCAG | 0.357603 | 0.357603 | 0.459988 | 0.37489 |
| | symmetric_AUUCAU-UCAUAC | CAACUCCCUCGCGC | | CAGCCACA | | | | |
| | 0_1-1_mismatch_A-C | AACUUUGAAAGUAU | | ACTCCCTCG | | | | |
| | 4_1-1_mismatch_A-G | UUCAUGAAUACAGC | | CGCAACTTT | | | | |
| | 18_2-1_bulge-asymmetric_GG-A | CACGGCUAUCAUAC | | GAAAGTAT | | | | |
| | 30_6-6_internal_loop- | UCCUUUACACCACA | | TTCATGAAT | | | | |
| | symmetric_GCCAAG-GCGCAA | CUGUCGUCGAAUGG | | ACAGCCAC | | | | |
| | | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 247 | -8_6-6_internal_loop- | CUCAGCAGCAGCCA | 686 | CTCAGCAG | 0.364482 | 0.364482 | 0.457145 | 0.373653 |
| | symmetric_GAAUUC-CGCCGA | CAACUCCCUCCUAU | | CAGCCACA | | | | |
| | 0_1-1_mismatch_A-C | AGAGUUGAAAGGUU | | ACTCCCTCC | | | | |
| | 2_1-1_wobble_G-U | UCAUCAAUACAUCU | | TATAGAGTT | | | | |
| | 11_1-1_mismatch_C-C | ACGGCUAAUCGCCG | | GAAAGGTT | | | | |
| | 18_3-1_bulge-asymmetric_GGA-G | ACUUUACACCACAC | | TCATCAATA | | | | |
| | 28_6-6_internal_loop- | UGUCGUCGAAUGGC | | CATCTACGG | | | | |
| | symmetric_AGGCCA-AUAGAG | | | CTAATCGCC | | | | |
| | | | | GACTTTACA | | | | |
| | | | | CCACACTGT | | | | |
| | | | | CGTCGAAT | | | | |
| | | | | GGC | | | | |
| 248 | -6_6-6_internal_loop- | CUCAGCAGCAGCCA | 687 | CTCAGCAG | 0.360703 | 0.360703 | 0.44969 | 0.372726 |
| | symmetric_AUUCAU-UCAUAC | CAACUCCCUCGCGC | | CAGCCACA | | | | |
| | 0_1-1_mismatch_A-C | AACUUUGAAAGGUU | | ACTCCCTCG | | | | |
| | 7_2-2_bulge-symmetric_UA-CC | UCAUGAACCCAUCC | | CGCAACTTT | | | | |
| | 18_3-1_bulge-asymmetric_GGA-G | ACGGCUAUCAUACU | | GAAAGGTT | | | | |
| | 30_6-6_internal_loop- | CCUUUACACCACAC | | TCATGAACC | | | | |
| | symmetric_GCCAAG-GCGCAA | UGUCGUCGAAUGGC | | CATCCACG | | | | |
| | | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 249 | -6_6-6_internal_loop- | CUCAGCAGCAGCCA | 688 | CTCAGCAG | 0.365405 | 0.365405 | 0.438813 | 0.362656 |
| | symmetric_AUUCAU-UCAUAC | CAACUCCCUCGCGC | | CAGCCACA | | | | |
| | 0_1-1_mismatch_A-C | AACUUUGAAAGGUU | | ACTCCCTCG | | | | |
| | 4_1-1_mismatch_A-G | UCAUGAAUACAGCC | | CGCAACTTT | | | | |
| | 18_3-1_bulge-asymmetric_GGA-G | ACGGCUAUCAUACU | | GAAAGGTT | | | | |
| | 30_6-6_internal_loop- | CCUUUACACCACAC | | TCATGAATA | | | | |
| | symmetric_GCCAAG-GCGCAA | UGUCGUCGAAUGGC | | CAGCCACG | | | | |
| | | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 250 | -6_6-6_internal_loop- | CUCAGCAGCAGCCA | 689 | CTCAGCAG | 0.366473 | 0.366473 | 0.435599 | 0.368349 |
| | symmetric_AUUCAU-UCAUAC | CAACUCCCUCGCGC | | CAGCCACA | | | | |
| | 0_1-1_mismatch_A-C | AACAUUGAAAGUAU | | ACTCCCTCG | | | | |
| | 3_1-2_bulge-asymmetric_G-AG | UUCAUGAAUACAUA | | CGCAACATT | | | | |
| | 18_2-1_bulge-asymmetric_GG-A | GCACGGCUAUCAUA | | GAAAGTAT | | | | |
| | 28_1-1_mismatch_A-A | CUCCUUUACACCAC | | TTCATGAAT | | | | |
| | 30_6-6_internal_loop- | ACUGUCGUCGAAUG | | ACATAGCA | | | | |
| | symmetric_GCCAAG-GCGCAA | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 251 | -6_6-6_internal_loop- | CUCAGCAGCAGCCA | 690 | CTCAGCAG | 0.360278 | 0.360278 | 0.452183 | 0.352939 |
| | symmetric_AUUCAU-UCAUAC | CAACUCCCUCGCGC | | CAGCCACA | | | | |
| | 0_1-1_mismatch_A-C | AACUUUGAAAGUCC | | ACTCCCTCG | | | | |
| | 8_2-2_bulge-symmetric_AU-UA | AGUCAUGAUAACAU | | CGCAACTTT | | | | |
| | 16_2-2_bulge-symmetric_AA-AG | CCACGGCUAUCAUA | | GAAAGTCC | | | | |
| | 30_6-6_internal_loop- | CUCCUUUACACCAC | | AGTCATGAT | | | | |
| | symmetric_GCCAAG-GCGCAA | ACUGUCGUCGAAUG | | AACATCCA | | | | |
| | | GC | | CGGCTATCA | | | | |
| | | | | TACTCCTTT | | | | |
| | | | | ACACCACA | | | | |
| | | | | CTGTCGTCG | | | | |
| | | | | AATGGC | | | | |
| 252 | -10_6-6_internal_loop- | CUCAGCAGCAGCCA | 691 | CTCAGCAG | 0.357861 | 0.357861 | 0.464615 | 0.401677 |
| | symmetric_AGGAAU-UAGAGC | CAACUCCCUCCUCU | | CAGCCACA | | | | |
| | -6_2-2_bulge-symmetric_AU-CG | CGAGUUGAAAGUGU | | ACTCCCTCC | | | | |
| | 0_1-1_mismatch_A-C | UUCAUGCUUACAUC | | TCTCGAGTT | | | | |
| | 9_2-2_bulge-symmetric_UU-CU | CACGGCUACGGAUA | | GAAAGTGT | | | | |
| | 18_2-1_bulge-asymmetric_GG-G | GAGCUUACACCACA | | TTCATGCTT | | | | |
| | 28_6-6_internal_loop- | CUGUCGUCGAAUGG | | ACATCCAC | | | | |
| | symmetric_AGGCCA-CUCGAG | C | | GGCTACGG | | | | |
| | | | | ATAGAGCTT | | | | |
| | | | | ACACCACA | | | | |
| | | | | CTGTCGTCG | | | | |
| | | | | AATGGC | | | | |
| 253 | -8_6-6_internal_loop- | CUCAGCAGCAGCCA | 692 | CTCAGCAG | 0.373268 | 0.373268 | 0.455275 | 0.396423 |
| | symmetric_GAAUUC-CGCCGA | CAACUCCCUCGCGC | | CAGCCACA | | | | |
| | -4_1-0_bulge-asymmetric_A- | AACUUUGAUGCCUU | | ACTCCCTCG | | | | |
| | 0_1-1_mismatch_A-C | UCAUUAUAACAUCC | | CGCAACTTT | | | | |
| | 8_4-4_bulge-symmetric_AUUC-UAUA | ACGGCAAUCGCCGA | | GATGCCTTT | | | | |
| | 20_4-2_bulge-asymmetric_ACUU-UG | CUUUACACCACACU | | CATTATAAC | | | | |
| | 30_6-6_internal_loop- | GUCGUCGAAUGGC | | ATCCACGG | | | | |
| | symmetric_GCCAAG-GCGCAA | | | CAATCGCC | | | | |
| | | | | GACTTTACA | | | | |
| | | | | CCACACTGT | | | | |
| | | | | CGTCGAAT | | | | |
| | | | | GGC | | | | |
| 254 | -6_6-6_internal_loop- | CUCAGCAGCAGCCA | 693 | CTCAGCAG | 0.347673 | 0.347673 | 0.480352 | 0.389378 |
| | symmetric_AUUCAU-UCAUAC | CAACUCCCUCGCGC | | CAGCCACA | | | | |
| | 0_1-1_mismatch_A-C | AACUUCAAGCACUU | | ACTCCCTCG | | | | |
| | 9_1-1_mismatch_U-C | UCAUGACUACAUCC | | CGCAACTTC | | | | |
| | 19_2-2_bulge-symmetric_GA-CA | ACGGCUAUCAUACU | | AAGCACTTT | | | | |
| | 24_3-1_bulge-asymmetric_UCA-C | CCUUUACACCACAC | | CATGACTAC | | | | |
| | 30_6-6_internal_loop- | UGUCGUCGAAUGGC | | ATCCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 255 | -6_6-6_internal_loop- | CUCAGCAGCAGCCA | 694 | CTCAGCAG | 0.348518 | 0.348518 | 0.468991 | 0.383226 |
| | symmetric_AUUCAU-UCAUAC | CAACUCCCUCGCGC | | CAGCCACA | | | | |
| | 0_1-1_mismatch_A-C | AACUUAUUAAGUCC | | ACTCCCTCG | | | | |
| | 2_1-1_wobble_G-U | UUUCAUGAAGCCAU | | CGCAACTTA | | | | |
| | 7_2-2_bulge-symmetric_UA-GC | CUACGGCUAUCAUA | | TTAAGTCCT | | | | |
| | 24_3-3_bulge-symmetric_UCA-AUU | CUCCUUUACACCAC | | TTCATGAAG | | | | |
| | 30_6-6_internal_loop- | ACUGUCGUCGAAUG | | CCATCTACG | | | | |
| | symmetric_GCCAAG-GCGCAA | GC | | GCTATCATA | | | | |
| | | | | CTCCTTTAC | | | | |
| | | | | ACCACACT | | | | |
| | | | | GTCGTCGA | | | | |
| | | | | ATGGC | | | | |
| 256 | -6_6-6_internal_loop- | CUCAGCAGCAGCCA | 695 | CTCAGCAG | 0.344474 | 0.344474 | 0.482569 | 0.395628 |
| | symmetric_AUUCAU-UCAUAC | CAACUCCCUCGCGC | | CAGCCACA | | | | |
| | 0_1-1_mismatch_A-C | AACUUUGAAAGUCA | | ACTCCCTCG | | | | |
| | 4_1-1_mismatch_A-A | UUCAUGUAUACAAC | | CGCAACTTT | | | | |
| | 10_1-1_mismatch_U-U | CACGGCUAUCAUAC | | GAAAGTCA | | | | |
| | 17_2-1_bulge-asymmetric_AG-A | UCCUUUACACCACA | | TTCATGTAT | | | | |
| | 30_6-6_internal_loop- | CUGUCGUCGAAUGG | | ACAACCAC | | | | |
| | symmetric_GCCAAG-GCGCAA | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 257 | -10_6-6_internal_loop- | CUCAGCAGCAGCCA | 696 | CTCAGCAG | 0.354208 | 0.354208 | 0.479149 | 0.411124 |
| | symmetric_AGGAAU-UAGAGC | CAACUCCCUCCUCU | | CAGCCACA | | | | |
| | -6_2-2_bulge-symmetric_AU-CG | CGAGUUGAAAGUCC | | ACTCCCTCC | | | | |
| | 0_1-1_mismatch_A-C | UGGCAUGAAUACAC | | TCTCGAGTT | | | | |
| | 4_1-0_bulge-asymmetric_A- | CACGGCUACGGAUA | | GAAAGTCC | | | | |
| | 15_2-2_bulge-symmetric_AA-GG | GAGCUUACACCACA | | TGGCATGA | | | | |
| | 28_6-6_internal_loop- | CUGUCGUCGAAUGG | | ATACACCA | | | | |
| | symmetric_AGGCCA-CUCGAG | C | | CGGCTACG | | | | |
| | | | | GATAGAGC | | | | |
| | | | | TTACACCAC | | | | |
| | | | | ACTGTCGTC | | | | |
| | | | | GAATGGC | | | | |
| 258 | -6_6-6_internal_loop- | CUCAGCAGCAGCCA | 697 | CTCAGCAG | 0.34192 | 0.34192 | 0.488377 | 0.387386 |
| | symmetric_AUUCAU-UCAUAC | CAACUCCCUCGCGC | | CAGCCACA | | | | |
| | 0_1-1_mismatch_A-C | AACUUUACCAAAAG | | ACTCCCTCG | | | | |
| | 9_3-3_bulge-symmetric_UUC-CUU | GGGUUUCAUCUUUA | | CGCAACTTT | | | | |
| | 18_3-3_bulge-symmetric_GGA-GGG | CAUCCACGGCUAUC | | ACCAAAAG | | | | |
| | 25_1-4_bulge-asymmetric_C-ACCA | AUACUCCUUUACAC | | GGGTTTCAT | | | | |
| | 30_6-6_internal_loop- | CACACUGUCGUCGA | | CTTTACATC | | | | |
| | symmetric_GCCAAG-GCGCAA | AUGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 259 | -6_6-6_internal_loop- | CUCAGCAGCAGCCA | 698 | CTCAGCAG | 0.343971 | 0.343971 | 0.464126 | 0.348641 |
| | symmetric_AUUCAU-UCAUAC | CAACUCCCUCGCGC | | CAGCCACA | | | | |
| | 0_1-1_mismatch_A-C | AACUUUAUAAAGUC | | ACTCCCTCG | | | | |
| | 4_1-0_bulge-asymmetric_A- | CUUUGGAUGAAUAC | | CGCAACTTT | | | | |
| | 14_1-2_bulge-asymmetric_G-GG | ACCACGGCUAUCAU | | ATAAAGTC | | | | |
| | 25_1-2_bulge-asymmetric_C-AU | ACUCCUUUACACCA | | CTTTGGATG | | | | |
| | 30_6-6_internal_loop- | CACUGUCGUCGAAU | | AATACACC | | | | |
| | symmetric_GCCAAG-GCGCAA | GGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 260 | -6_6-6_internal_loop- | CUCAGCAGCAGCCA | 699 | CTCAGCAG | 0.350386 | 0.350386 | 0.440942 | 0.372333 |
| | symmetric_AUUCAU-UCAUAC | CAACUCCCUCGCGC | | CAGCCACA | | | | |
| | 0_1-1_mismatch_A-C | AACUUUGACAGUAU | | ACTCCCTCG | | | | |
| | 9_2-2_bulge-symmetric_UU-UU | UCAUGUUUACAUCC | | CGCAACTTT | | | | |
| | 17_3-1_bulge-asymmetric_AGG-A | ACGGCUAUCAUACU | | GACAGTATT | | | | |
| | 23_1-1_mismatch_U-C | CCUUUACACCACAC | | CATGTTTAC | | | | |
| | 30_6-6_internal_loop- | UGUCGUCGAAUGGC | | ATCCACGG | | | | |
| | symmetric_GCCAAG-GCGCAA | | | CTATCATAC | | | | |
| | | | | TCCTTTACA | | | | |
| | | | | CCACACTGT | | | | |
| | | | | CGTCGAAT | | | | |
| | | | | GGC | | | | |
| 261 | −18_6-6_internal_loop- | CUCAGCAGCAGCCA | 700 | CTCAGCAG | 0.354955 | 0.354955 | 0.439032 | 0.398742 |
| | symmetric_UGGUGU-UGGAGU | CAACUCCCUCGCGC | | CAGCCACA | | | | |
| | -6_3-3_bulge-symmetric_CAU-CAU | AACUUUGAAAGCAA | | ACTCCCTCG | | | | |
| | 0_1-1_mismatch_A-C | UUUCAUGCAUAGAC | | CGCAACTTT | | | | |
| | 4_1-0_bulge-asymmetric_A- | CACGGCUACAUAAU | | GAAAGCAA | | | | |
| | 6_1-1_mismatch_G-G | UCCUUUUGGAGUCA | | TTTCATGCA | | | | |
| | 10_1-1_mismatch_U-C | CUGUCGUCGAAUGG | | TAGACCAC | | | | |
| | 18_3-3_bulge-symmetric_GGA-CAA | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 262 | -6_6-6_internal_loop- | CUCAGCAGCAGCCA | 701 | CTCAGCAG | 0.35672 | 0.35672 | 0.43618 | 0.35884 |
| | symmetric_AUUCAU-UCAUAC | CAACUCCCUCGCGC | | CAGCCACA | | | | |
| | 0_1-1_mismatch_A-C | AACUUUUCCAAAGU | | ACTCCCTCG | | | | |
| | 3_2-2_bulge-symmetric_GA-AG | CCUUUGAUGAAUAC | | CGCAACTTT | | | | |
| | 14_1-1_mismatch_G-G | AAGCACGGCUAUCA | | TCCAAAGTC | | | | |
| | 25_1-3_bulge-asymmetric_C-UCC | UACUCCUUUACACC | | CTTTGATGA | | | | |
| | 30_6-6_internal_loop- | ACACUGUCGUCGAA | | ATACAAGC | | | | |
| | symmetric_GCCAAG-GCGCAA | UGGC | | ACGGCTATC | | | | |
| | | | | ATACTCCTT | | | | |
| | | | | TACACCAC | | | | |
| | | | | ACTGTCGTC | | | | |
| | | | | GAATGGC | | | | |
| 263 | -10_6-6_internal_loop- | CUCAGCAGCAGCCA | 377 | CTCAGCAG | 0.353038 | 0.353038 | 0.462143 | 0.409117 |
| | symmetric_AGGAAU-UCUGAG | CAACUCCCUCCUGA | | CAGCCACA | | | | |
| | -4_2-0_bulge-asymmetric_UA- | AUGAUUGAAAGUCC | | ACTCCCTCC | | | | |
| | 0_1-1_mismatch_A-C | UUUCAUGAAUACAU | | TGAATGATT | | | | |
| | 28_6-6_internal_loop- | CCACGGCAUGAUCU | | GAAAGTCC | | | | |
| | symmetric_AGGCCA-GAAUGA | GAGUUACACCACAC | | TTTCATGAA | | | | |
| | | UGUCGUCGAAUGGC | | TACATCCAC | | | | |
| | | | | GGCATGAT | | | | |
| | | | | CTGAGTTAC | | | | |
| | | | | ACCACACT | | | | |
| | | | | GTCGTCGA | | | | |
| | | | | ATGGC | | | | |
| 264 | -6_6-6_internal_loop- | CUCAGCAGCAGCCA | 702 | CTCAGCAG | 0.344502 | 0.344502 | 0.451624 | 0.369981 |
| | symmetric_AUUCAU-UCAUAC | CAACUCCCUCGCGC | | CAGCCACA | | | | |
| | 0_1-1_mismatch_A-C | AACUUUGAAAUAUU | | ACTCCCTCG | | | | |
| | 4_1-0_bulge-asymmetric_A- | UCAUCUCUACACCA | | CGCAACTTT | | | | |
| | 9_3-3_bulge-symmetric_UUC-CUC | CGGCUAUCAUACUC | | GAAATATTT | | | | |
| | 18_4-2_bulge-asymmetric_GGAC-UA | CUUUACACCACACU | | CATCTCTAC | | | | |
| | 30_6-6_internal_loop- | GUCGUCGAAUGGC | | ACCACGGC | | | | |
| | symmetric_GCCAAG-GCGCAA | | | TATCATACT | | | | |
| | | | | CCTTTACAC | | | | |
| | | | | CACACTGTC | | | | |
| | | | | GTCGAATG | | | | |
| | | | | GC | | | | |
| 265 | -8_6-6_internal_loop- | CUCAGCAGCAGCCA | 703 | CTCAGCAG | 0.34072 | 0.34072 | 0.470101 | 0.427055 |
| | symmetric_GAAUUC-CGCCGA | CAACUCCCUCGCGC | | CAGCCACA | | | | |
| | -4_1-0_bulge-asymmetric_A- | AACUUUGAAACUCU | | ACTCCCTCG | | | | |
| | 0_1-1_mismatch_A-C | UCCUUUCAUGCUGC | | CGCAACTTT | | | | |
| | 8_3-4_bulge-asymmetric_AUU-CUGC | ACAUCCACGGCAAU | | GAAACTCTT | | | | |
| | 21_1-4_bulge-asymmetric_C-CUCU | CGCCGACUUUACAC | | CCTTTCATG | | | | |
| | 30_6-6_internal_loop- | CACACUGUCGUCGA | | CTGCACATC | | | | |
| | symmetric_GCCAAG-GCGCAA | AUGGC | | CACGGCAA | | | | |
| | | | | TCGCCGACT | | | | |
| | | | | TTACACCAC | | | | |
| | | | | ACTGTCGTC | | | | |
| | | | | GAATGGC | | | | |
| 266 | -6_6-6_internal_loop- | CUCAGCAGCAGCCA | 704 | CTCAGCAG | 0.344254 | 0.344254 | 0.451534 | 0.354701 |
| | symmetric_AUUCAU-UCAUAC | CAACUCCCUCGCGC | | CAGCCACA | | | | |
| | 0_1-1_mismatch_A-C | AACUUUGAAAGAUU | | ACTCCCTCG | | | | |
| | 2_1-1_wobble_G-U | UCAUGAAUAGAUCU | | CGCAACTTT | | | | |
| | 6_1-1_mismatch_G-G | ACGGCUAUCAUACU | | GAAAGATT | | | | |
| | 18_3-1_bulge-asymmetric_GGA-A | CCUUUACACCACAC | | TCATGAATA | | | | |
| | 30_6-6_internal_loop- | UGUCGUCGAAUGGC | | GATCTACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 267 | -8_6-6_internal_loop- | CUCAGCAGCAGCCA | 705 | CTCAGCAG | 0.338848 | 0.338848 | 0.437992 | 0.382641 |
| | symmetric_GAAUUC-CGCCGA | CAACUCCCUCCUAU | | CAGCCACA | | | | |
| | 0_1-1_mismatch_A-C | AGAGUUGUGUCCUU | | ACTCCCTCC | | | | |
| | 2_1-1_wobble_G-U | UCAUGUAUACAUCU | | TATAGAGTT | | | | |
| | 10_1-1_mismatch_U-U | ACGGCUAAUCGCCG | | GTGTCCTTT | | | | |
| | 22_3-1_bulge-asymmetric_UUU-U | ACUUUACACCACAC | | CATGTATAC | | | | |
| | 28_6-6_internal_loop- | UGUCGUCGAAUGGC | | ATCTACGGC | | | | |
| | symmetric_AGGCCA-AUAGAG | | | TAATCGCCG | | | | |
| | | | | ACTTTACAC | | | | |
| | | | | CACACTGTC | | | | |
| | | | | GTCGAATG | | | | |
| | | | | GC | | | | |
| 268 | -6_6-6_internal_loop- | CUCAGCAGCAGCCA | 706 | CTCAGCAG | 0.338806 | 0.338806 | 0.471768 | 0.369025 |
| | symmetric_AUUCAU-UCAUAC | CAACUCCCUCGCGC | | CAGCCACA | | | | |
| | 0_1-1_mismatch_A-C | AACUUCAGGAAAGA | | ACTCCCTCG | | | | |
| | 2_1-1_wobble_G-U | UUUCAUGAAUACAU | | CGCAACTTC | | | | |
| | 18_3-1_bulge-asymmetric_GGA-A | CUACGGCUAUCAUA | | AGGAAAGA | | | | |
| | 26_1-3_bulge-asymmetric_A-CAG | CUCCUUUACACCAC | | TTTCATGAA | | | | |
| | 30_6-6_internal_loop- | ACUGUCGUCGAAUG | | TACATCTAC | | | | |
| | symmetric_GCCAAG-GCGCAA | GC | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 269 | -6_6-6_internal_loop- | CUCAGCAGCAGCCA | 707 | CTCAGCAG | 0.350133 | 0.350133 | 0.431509 | 0.36214 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 4_2-2_bulge-symmetric_AU-UG | UUCCUGAAUACUGC | | CGCAACTTT | | | | |
| | 13_1-1_mismatch_U-C | CACGGCUAUCAUAC | | GAAAGTGT | | | | |
| | 18_2-1_bulge-asymmetric_GG-G | UCCUUUACACCACA | | TTCCTGAAT | | | | |
| | 30_6-6_internal_loop- | CUGUCGUCGAAUGG | | ACTGCCAC | | | | |
| | symmetric_GCCAAG-GCGCAA | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 270 | −18_6-6_internal_loop- | CUCAGCAGCAGCCA | 708 | CTCAGCAG | 0.343193 | 0.343193 | 0.455386 | 0.371063 |
| | symmetric_UGGUGU-UGGAGU | CAACUCCCUCGCGC | | CAGCCACA | | | | |
| | -6_3-3_bulge-symmetric_CAU-CAU | AACUUUGAAAGUGG | | ACTCCCTCG | | | | |
| | 0_1-1_mismatch_A-C | CUUCAUGCAUAGAC | | CGCAACTTT | | | | |
| | 4_1-0_bulge-asymmetric_A- | CACGGCUACAUAAU | | GAAAGTGG | | | | |
| | 6_1-1_mismatch_G-G | UCCUUUUGGAGUCA | | CTTCATGCA | | | | |
| | 10_1-1_mismatch_U-C | CUGUCGUCGAAUGG | | TAGACCAC | | | | |
| | 17_3-3_bulge-symmetric_AGG-GGC | C | | GGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 271 | -8_6-6_internal_loop- | CUCAGCAGCAGCCA | 709 | CTCAGCAG | 0.336571 | 0.336571 | 0.471134 | 0.373521 |
| | symmetric_GAAUUC-CGCCGA | CAACUCCCUCCUAU | | CAGCCACA | | | | |
| | 0_1-1_mismatch_A-C | AGAGUUGAAAGACU | | ACTCCCTCC | | | | |
| | 2_1-1_wobble_G-U | UUCAUGUCUACAUC | | TATAGAGTT | | | | |
| | 9_2-2_bulge-symmetric_UU-UC | UACGGCUAAUCGCC | | GAAAGACT | | | | |
| | 19_2-1_bulge-asymmetric_GA-A | GACUUUACACCACA | | TTCATGTCT | | | | |
| | 28_6-6_internal_loop- | CUGUCGUCGAAUGG | | ACATCTACG | | | | |
| | symmetric_AGGCCA-AUAGAG | C | | GCTAATCGC | | | | |
| | | | | CGACTTTAC | | | | |
| | | | | ACCACACT | | | | |
| | | | | GTCGTCGA | | | | |
| | | | | ATGGC | | | | |
| 272 | -8_6-6_internal_loop- | CUCAGCAGCAGCCA | 710 | CTCAGCAG | 0.348823 | 0.348823 | 0.436216 | 0.359507 |
| | symmetric_GAAUUC-CGCCGA | CAACUCCCUCCUAU | | CAGCCACA | | | | |
| | 0_1-1_mismatch_A-C | AGAGUUGACCCUCC | | ACTCCCTCC | | | | |
| | 2_1-1_wobble_G-U | UUUCAUGAAUACCG | | TATAGAGTT | | | | |
| | 4_2-2_bulge-symmetric_AU-CG | CUACGGCUAAUCGC | | GACCCTCCT | | | | |
| | 21_3-3_bulge-symmetric_CUU-CCC | CGACUUUACACCAC | | TTCATGAAT | | | | |
| | 28_6-6_internal_loop- | ACUGUCGUCGAAUG | | ACCGCTAC | | | | |
| | symmetric_AGGCCA-AUAGAG | GC | | GGCTAATC | | | | |
| | | | | GCCGACTTT | | | | |
| | | | | ACACCACA | | | | |
| | | | | CTGTCGTCG | | | | |
| | | | | AATGGC | | | | |
| 273 | -6_6-6_internal_loop- | CUCAGCAGCAGCCA | 711 | CTCAGCAG | 0.342992 | 0.342992 | 0.461253 | 0.363877 |
| | symmetric_AUUCAU-UCAUAC | CAACUCCCUCGCGC | | CAGCCACA | | | | |
| | 0_1-1_mismatch_A-C | AACUUUGAAAGUCG | | ACTCCCTCG | | | | |
| | 2_1-1_wobble_G-U | UUCAUGACGCCAUC | | CGCAACTTT | | | | |
| | 7_3-3_bulge-symmetric_UAU-CGC | UACGGCUAUCAUAC | | GAAAGTCG | | | | |
| | 17_2-1_bulge-asymmetric_AG-G | UCCUUUACACCACA | | TTCATGACG | | | | |
| | 30_6-6_internal_loop- | CUGUCGUCGAAUGG | | CCATCTACG | | | | |
| | symmetric_GCCAAG-GCGCAA | C | | GCTATCATA | | | | |
| | | | | CTCCTTTAC | | | | |
| | | | | ACCACACT | | | | |
| | | | | GTCGTCGA | | | | |
| | | | | ATGGC | | | | |
| 274 | -10_6-6_internal_loop- | CUCAGCAGCAGCCA | 712 | CTCAGCAG | 0.346369 | 0.346369 | 0.465576 | 0.390772 |
| | symmetric_AGGAAU-UAGAGC | CAACUCCCUCCUCU | | CAGCCACA | | | | |
| | -6_2-2_bulge-symmetric_AU-CG | CGAGUUGAAAGUGA | | ACTCCCTCC | | | | |
| | 0_1-1_mismatch_A-C | CUUCAUGAGUACAU | | TCTCGAGTT | | | | |
| | 9_1-1_wobble_U-G | CCACGGCUACGGAU | | GAAAGTGA | | | | |
| | 17_3-3_bulge-symmetric_AGG-GAC | AGAGCUUACACCAC | | CTTCATGAG | | | | |
| | 28_6-6_internal_loop- | ACUGUCGUCGAAUG | | TACATCCAC | | | | |
| | symmetric_AGGCCA-CUCGAG | GC | | GGCTACGG | | | | |
| | | | | ATAGAGCTT | | | | |
| | | | | ACACCACA | | | | |
| | | | | CTGTCGTCG | | | | |
| | | | | AATGGC | | | | |
| 275 | -6_6-6_internal_loop- | CUCAGCAGCAGCCA | 713 | CTCAGCAG | 0.338406 | 0.338406 | 0.449726 | 0.37465 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 2_1-1_wobble_G-U | UCAUGAAUAAAAUC | | CGCAACTTT | | | | |
| | 6_1-2_bulge-asymmetric_G-AA | UACGGCUAUCAUAC | | GAAAGTGT | | | | |
| | 17_3-1_bulge-asymmetric_AGG-G | UCCUUUACACCACA | | TCATGAATA | | | | |
| | 30_6-6_internal_loop- | CUGUCGUCGAAUGG | | AAATCTAC | | | | |
| | symmetric_GCCAAG-GCGCAA | C | | GGCTATCAT | | | | |
| | | | | ACTCCTTTA | | | | |
| | | | | CACCACACT | | | | |
| | | | | GTCGTCGA | | | | |
| | | | | ATGGC | | | | |
| 276 | -10_6-6_internal_loop- | CUCAGCAGCAGCCA | 714 | CTCAGCAG | 0.345953 | 0.345953 | 0.458812 | 0.400477 |
| | symmetric_AGGAAU-UAGAGC | CAACUCCCUCCUCU | | CAGCCACA | | | | |
| | -6_2-2_bulge-symmetric_AU-CG | CGAGUUGAAAGGGA | | ACTCCCTCC | | | | |
| | 0_1-1_mismatch_A-C | CCUUUCAUGAAUAC | | TCTCGAGTT | | | | |
| | 4_1-0_bulge-asymmetric_A- | ACCACGGCUACGGA | | GAAAGGGA | | | | |
| | 20_1-3_bulge-asymmetric_A-GGA | UAGAGCUUACACCA | | CCTTTCATG | | | | |
| | 28_6-6_internal_loop- | CACUGUCGUCGAAU | | AATACACC | | | | |
| | symmetric_AGGCCA-CUCGAG | GGC | | ACGGCTAC | | | | |
| | | | | GGATAGAG | | | | |
| | | | | CTTACACCA | | | | |
| | | | | CACTGTCGT | | | | |
| | | | | CGAATGGC | | | | |
| 277 | -6_6-6_internal_loop- | CUCAGCAGCAGCCA | 715 | CTCAGCAG | 0.342728 | 0.342728 | 0.438754 | 0.358336 |
| | symmetric_AUUCAU-UCAUAC | CAACUCCCUCGCGC | | CAGCCACA | | | | |
| | 0_1-1_mismatch_A-C | AACUUUGAACGUGU | | ACTCCCTCG | | | | |
| | 3_2-2_bulge-symmetric_GA-AG | CCUUUCAUGAAUAC | | CGCAACTTT | | | | |
| | 22_1-3_bulge-asymmetric_U-CGU | AAGCACGGCUAUCA | | GAACGTGT | | | | |
| | 30_6-6_internal_loop- | UACUCCUUUACACC | | CCTTTCATG | | | | |
| | symmetric_GCCAAG-GCGCAA | ACACUGUCGUCGAA | | AATACAAG | | | | |
| | | UGGC | | CACGGCTAT | | | | |
| | | | | CATACTCCT | | | | |
| | | | | TTACACCAC | | | | |
| | | | | ACTGTCGTC | | | | |
| | | | | GAATGGC | | | | |
| 278 | -10_6-6_internal_loop- | CUCAGCAGCAGCCA | 716 | CTCAGCAG | 0.341978 | 0.341978 | 0.457147 | 0.394494 |
| | symmetric_AGGAAU-UAGAGC | CAACUCCCUCCUCU | | CAGCCACA | | | | |
| | -6_2-2_bulge-symmetric_AU-CG | CGAGUUGAAAGUAG | | ACTCCCTCC | | | | |
| | 0_1-1_mismatch_A-C | AUUCAUGAUUACAU | | TCTCGAGTT | | | | |
| | 9_1-1_mismatch_U-U | CCACGGCUACGGAU | | GAAAGTAG | | | | |
| | 17_3-3_bulge-symmetric_AGG-AGA | AGAGCUUACACCAC | | ATTCATGAT | | | | |
| | 28_6-6_internal_loop- | ACUGUCGUCGAAUG | | TACATCCAC | | | | |
| | symmetric_AGGCCA-CUCGAG | GC | | GGCTACGG | | | | |
| | | | | ATAGAGCTT | | | | |
| | | | | ACACCACA | | | | |
| | | | | CTGTCGTCG | | | | |
| | | | | AATGGC | | | | |
| 279 | -6_6-6_internal_loop- | CUCAGCAGCAGCCA | 717 | CTCAGCAG | 0.340165 | 0.340165 | 0.43477 | 0.378264 |
| | symmetric_AUUCAU-UCAUAC | CAACUCCCUCGCGC | | CAGCCACA | | | | |
| | 0_1-1_mismatch_A-C | AACUUAACGAUCAG | | ACTCCCTCG | | | | |
| | 3_1-1_mismatch_G-G | UCCUUUCAUGAAUA | | CGCAACTTA | | | | |
| | 23_1-2_bulge-asymmetric_U-UC | CAUGCACGGCUAUC | | ACGATCAG | | | | |
| | 26_1-3_bulge-asymmetric_A-AAC | AUACUCCUUUACAC | | TCCTTTCAT | | | | |
| | 30_6-6_internal_loop- | CACACUGUCGUCGA | | GAATACAT | | | | |
| | symmetric_GCCAAG-GCGCAA | AUGGC | | GCACGGCT | | | | |
| | | | | ATCATACTC | | | | |
| | | | | CTTTACACC | | | | |
| | | | | ACACTGTCG | | | | |
| | | | | TCGAATGG | | | | |
| | | | | C | | | | |
| 280 | -6_6-6_internal_loop- | CUCAGCAGCAGCCA | 718 | CTCAGCAG | 0.35605 | 0.35605 | 0.422068 | 0.356886 |
| | symmetric_AUUCAU-UCAUAC | CAACUCCCUCGCGC | | CAGCCACA | | | | |
| | 0_1-1_mismatch_A-C | AACUUUGAACCCUU | | ACTCCCTCG | | | | |
| | 2_1-1_wobble_G-U | UCACAUACAUCUAC | | CGCAACTTT | | | | |
| | 10_3-1_bulge-asymmetric_UCA-C | GGCUAUCAUACUCC | | GAACCCTTT | | | | |
| | 20_3-1_bulge-asymmetric_ACU-C | UUUACACCACACUG | | CACATACAT | | | | |
| | 30_6-6_internal_loop- | UCGUCGAAUGGC | | CTACGGCTA | | | | |
| | symmetric_GCCAAG-GCGCAA | | | TCATACTCC | | | | |
| | | | | TTTACACCA | | | | |
| | | | | CACTGTCGT | | | | |
| | | | | CGAATGGC | | | | |
| 281 | −18_6-6_internal_loop- | CUCAGCAGCAGCCA | 719 | CTCAGCAG | 0.342303 | 0.342303 | 0.456585 | 0.368217 |
| | symmetric_UGGUGU-UGGAGU | CAACUCCCUCGCGC | | CAGCCACA | | | | |
| | -6_3-3_bulge-symmetric_CAU-CAU | AACUUUGAAAGUCC | | ACTCCCTCG | | | | |
| | 0_1-1_mismatch_A-C | GGUCAUGCAUAGAC | | CGCAACTTT | | | | |
| | 4_1-0_bulge-asymmetric_A- | CACGGCUACAUAAU | | GAAAGTCC | | | | |
| | 6_1-1_mismatch_G-G | UCCUUUUGGAGUCA | | GGTCATGC | | | | |
| | 10_1-1_mismatch_U-C | CUGUCGUCGAAUGG | | ATAGACCA | | | | |
| | 16_2-2_bulge-symmetric_AA-GG | C | | CGGCTACAT | | | | |
| | 30_6-6_internal_loop- | | | AATTCCTTT | | | | |
| | symmetric_GCCAAG-GCGCAA | | | TGGAGTCA | | | | |
| | | | | CTGTCGTCG | | | | |
| | | | | AATGGC | | | | |
| 282 | -4_6-6_internal_loop- | CUCAGCAGCAGCCA | 720 | CTCAGCAG | 0.343875 | 0.343875 | 0.452265 | 0.369938 |
| | symmetric_UCAUUA-GUCAUC | CAACUCCCUCCUGA | | CAGCCACA | | | | |
| | 0_1-1_mismatch_A-C | AUGAUUGAAAGUCC | | ACTCCCTCC | | | | |
| | 13_1-1_mismatch_U-C | UUCCCUGAAUACAU | | TGAATGATT | | | | |
| | 15_1-1_mismatch_A-C | CCACGGCGUCAUCA | | GAAAGTCC | | | | |
| | 28_6-6_internal_loop- | UUCCUUUACACCAC | | TTCCCTGAA | | | | |
| | symmetric_AGGCCA-GAAUGA | ACUGUCGUCGAAUG | | TACATCCAC | | | | |
| | | GC | | GGCGTCATC | | | | |
| | | | | ATTCCTTTA | | | | |
| | | | | CACCACACT | | | | |
| | | | | GTCGTCGA | | | | |
| | | | | ATGGC | | | | |
| 283 | -6_6-6_internal_loop- | CUCAGCAGCAGCCA | 721 | CTCAGCAG | 0.338434 | 0.338434 | 0.427857 | 0.369617 |
| | symmetric_AUUCAU-UCAUAC | CAACUCCCUCGCGC | | CAGCCACA | | | | |
| | 0_1-1_mismatch_A-C | AACUUUGAAAGUGU | | ACTCCCTCG | | | | |
| | 4_2-2_bulge-symmetric_AU-CG | UCAUGCCUACCGCC | | CGCAACTTT | | | | |
| | 9_2-2_bulge-symmetric_UU-CC | ACGGCUAUCAUACU | | GAAAGTGT | | | | |
| | 17_3-1_bulge-asymmetric_AGG-G | CCUUUACACCACAC | | TCATGCCTA | | | | |
| | 30_6-6_internal_loop- | UGUCGUCGAAUGGC | | CCGCCACG | | | | |
| | symmetric_GCCAAG-GCGCAA | | | GCTATCATA | | | | |
| | | | | CTCCTTTAC | | | | |
| | | | | ACCACACT | | | | |
| | | | | GTCGTCGA | | | | |
| | | | | ATGGC | | | | |
| 284 | -10_6-6_internal_loop- | CUCAGCAGCAGCCA | 722 | CTCAGCAG | 0.342755 | 0.342755 | 0.474225 | 0.413163 |
| | symmetric_AGGAAU-UAGAGC | CAACUCCCUCCUCU | | CAGCCACA | | | | |
| | -6_2-2_bulge-symmetric_AU-CG | CGAGUUGAAUCCCC | | ACTCCCTCC | | | | |
| | 0_1-1_mismatch_A-C | UUUCAUGAAUACAC | | TCTCGAGTT | | | | |
| | 4_1-0_bulge-asymmetric_A- | CACGGCUACGGAUA | | GAATCCCCT | | | | |
| | 20_3-3_bulge-symmetric_ACU-UCC | GAGCUUACACCACA | | TTCATGAAT | | | | |
| | 28_6-6_internal_loop- | CUGUCGUCGAAUGG | | ACACCACG | | | | |
| | symmetric_AGGCCA-CUCGAG | C | | GCTACGGA | | | | |
| | | | | TAGAGCTTA | | | | |
| | | | | CACCACACT | | | | |
| | | | | GTCGTCGA | | | | |
| | | | | ATGGC | | | | |
| 285 | -10_6-6_internal_loop- | CUCAGCAGCAGCCA | 723 | CTCAGCAG | 0.342678 | 0.342678 | 0.459303 | 0.385217 |
| | symmetric_AGGAAU-UAGAGC | CAACUCCCUCCUCU | | CAGCCACA | | | | |
| | -6_2-2_bulge-symmetric_AU-CG | CGAGUUGAAAGAAA | | ACTCCCTCC | | | | |
| | 0_1-1_mismatch_A-C | CCUUUCAUGAAUAC | | TCTCGAGTT | | | | |
| | 4_2-2_bulge-symmetric_AU-CA | CACCACGGCUACGG | | GAAAGAAA | | | | |
| | 20_1-3_bulge-asymmetric_A-AAA | AUAGAGCUUACACC | | CCTTTCATG | | | | |
| | 28_6-6_internal_loop- | ACACUGUCGUCGAA | | AATACCAC | | | | |
| | symmetric_AGGCCA-CUCGAG | UGGC | | CACGGCTA | | | | |
| | | | | CGGATAGA | | | | |
| | | | | GCTTACACC | | | | |
| | | | | ACACTGTCG | | | | |
| | | | | TCGAATGG | | | | |
| | | | | C | | | | |

Thus, Example 1 demonstrates production of engineered guide RNAs that target the Exon 2 Codon 1 TIS and facilitated editing of the TIS via ADAR1 and ADAR2 with high on-target editing and specificity.

Additional guide RNAs depicted in Table 2 below were engineered to target the Exon 2 translation initiation site (TIS) of SNCA mRNA in order to reduce levels of full-length alpha-synuclein protein levels. The guide RNAs depicted in Table 2 below were utilized in the Examples that follow.

TABLE 2
SNCA-Targeting Sequences.

| SEQ ID | | SEQ ID | | |
|---|---|---|---|---|
| NO: | Guide DNA Sequence | NO: | Corresponding Guide RNA sequence | Structural Features |
| 286 | CAGCAGCCACAACTCCCTCCTA | 293 | CAGCAGCCACAACUCCCUCCUAUAGA | -8_6-6_internal_loop-symmetric_GAAUUC-CGCCGA |
| | TAGAGTTGAAAGTCCTTTCATG | | GUUGAAAGUCCUUUCAUGAAUACAUC | 0_1-1_mismatch_A-C |
| | AATACATCTACGGCTAATCGCC | | UACGGCUAAUCGCCGACUUUACACCA | 2_1-1_wobble_G-U |
| | GACTTTACACCACACTGTCGTC | | CACUGUCGUCGAAUGGC | 28_6-6_internal_loop-symmetric_AGGCCA-AUAGAG |
| | GAATGGC | | | |
| 287 | AGCAGCCACAACTCCCTCCTAC | 294 | AGCAGCCACAACUCCCUCCUACAAAC | -10_6-6_internal_loop-symmetric_AGGAAU-CGGAGA |
| | AAACTTGAAAGTCCTTTCATGA | | UUGAAAGUCCUUUCAUGAAUACAACC | -6_0-1_bulge-asymmetric_-U |
| | ATACAACCACGGCTAATTGACG | | ACGGCUAAUUGACGGAGAUUACACCA | 0_1-1_mismatch_A-C |
| | GAGATTACACCACACTGTC | | CACUGUC | 4_1-1_mismatch_A-A |
| | | | | 28_6-6_internal_loop-symmetric_AGGCCA-ACAAAC |
| 288 | CAGCAGCCACAACTCCCAGGAT | 295 | CAGCAGCCACAACUCCCAGGAUCGCC | -6_6-6_internal_loop-symmetric_AUUCAU-CGACAG |
| | CGCCTTTGAAAGTCCTTCCCTGA | | UUUGAAAGUCCUUCCCUGAAUACAUC | 0_1-1_mismatch_A-C |
| | ATACATCCACGGCTACGACAGT | | CACGGCUACGACAGUCCUUUACACCA | 13_1-1_mismatch_U-C |
| | CCTTTACACCACACTGTCGTCG | | CACUGUCGUCGAAUGGC | 15_1-1_mismatch_A-C |
| | AATGGC | | | 32_6-6_internal_loop-symmetric_CAAGGA-AGGAUC |
| 289 | GCCACAACTCCCTCCTTGATAG | 296 | GCCACAACUCCCUCCUUGAUAGGCGA | -6_6-6_internal_loop-symmetric_AUUCAU-UCAUAC |
| | GCGAAAGTCCTTTCATGAATAA | | AAGUCCUUUCAUGAAUAACUCCACGG | 0_1-1_mismatch_A-C |
| | CTCCACGGCTATCATACTCCTTT | | CUAUCAUACUCCUUUACACCACACUG | 5_2-2_bulge-symmetric_UG-AC |
| | ACACCACACTGTCGTCGA | | UCGUCGA | 26_6-6_internal_loop-symmetric_AAAGGC-AUAGG |
| 290 | CAGCAGCCACAACTCCCTCGCG | 297 | CAGCAGCCACAACUCCCUCGCGCAAC | -18_6-6_internal_loop-symmetric_UGGUGU-UGGAGU |
| | CAACTTTGAAAGTCCTTTCATGC | | UUUGAAAGUCCUUUCAUGCAUAGAUC | -6_3-3_bulge-symmetric_CAU-CAU |
| | ATAGATCCACGGCTACATAATT | | CACGGCUACAUAAUUCCUUUUGGAGU | 0_1-1_mismatch_A-C |
| | CCTTTTGGAGTCACTGTCGTCGA | | CACUGUCGUCGA | 6_1-1_mismatch_G-G |
| | | | | 10_1-1_mismatch_U-C |
| | | | | 30_6-6_internal_loop-symmetric_GCCAAG-GCGCAA |
| 291 | CTCAGCAGCAGCCACAACTCCA | 298 | CUCAGCAGCAGCCACAACUCCAAUGA | -6_6-6_internal_loop-symmetric_AUUCAU-CAUAUC |
| | ATGAAGGCCTTTGAAAGTCCTT | | AGGCCUUUGAAAGUCCUUUCAUGAAU | 0_1-1_mismatch_A-C |
| | TCATGAATACATCTACGGCTAC | | ACAUCUACGGCUACAUAUCUCCUUUA | 2_1-1_wobble_G-U |
| | ATATCTCCTTTACACCACACTGT | | CACCACACUGUCGUCGAAUGGC | 33_6-6_internal_loop-symmetric_AAGGAG-AAUGAA |
| | CGTCGAATGGC | | | |
| 292 | CTCAGCAGCAGCCACAACTCCA | 299 | CUCAGCAGCAGCCACAACUCCAGAAU | -6_6-6_internal_loop-symmetric_AUUCAU-CAUAUC |
| | GAATAGGCCTTTGAAAGTCCTT | | AGGCCUUUGAAAGUCCUUUCAUGAAU | 0_1-1_mismatch_A-C |
| | TCATGAATAACTCCACGGCTAT | | AACUCCACGGCUAUAAAUCUCCUUUA | 2_1-1_wobble_G-U |
| | AAATCTCCTTTACACCACACTGT | | CACCACACUGUCGUCGAAUGGC | 33_6-6_internal_loop-symmetric_AAGGAG-AAUGAA |
| | CGTCGAATGGC | | | |
| 325 | CAGCAGCCACAACTCCCGAAGG | 333 | CAGCAGCCACAACUCCCGAAGGAGCC | -6_6-6_internal_loop-symmetric_AUUCAU-UACUUA |
| | AGCCTTTGAAAGTCCTTAATTG | | UUUGAAAGUCCUUAAUUGAAUACAUC | 0_1-1_mismatch_A-C |
| | AATACATCCACGGCTATACTTA | | CACGGCUAUACUUAUCCUUUACACCA | 13_3-3_bulge-symmetric_UGA-AAU |
| | TCCTTTACACCACACTGTCGTCG | | CACUGUCGUCGAAUGGC | 31_0-1_bulge-asymmetric_-A |
| | AATGGC | | | 33_5-4_internal_loop-asymmetric_AAGGA-GAAG |
| | | | | 55_1-1_mismatch_C-A |
| 326 | CAGCAGCCACAACTCCCGAAGG | 334 | CAGCAGCCACAACUCCCGAAGGAGCC | -40_1-1_wobble_G-U |
| | AGCCTTTGAAAGTCCTTAATTG | | UUUGAAAGUCCUUAAUUGAAUACAUC | -36_1-1_wobble_U-G |
| | AATACATCCACGGCTATACTTA | | CACGGCUAUACUUAUCCUUUACACCA | -33_1-1_wobble_G-U |
| | TCCTTTACACCACACTGTCGTTG | | CACUGUCGUUGAGUGGU | -6_6-6_internal_loop-symmetric_AUUCAU-UACUUA |
| | AGTGGT | | | 0_1-1_mismatch_A-C |
| | | | | 13_3-3_bulge-symmetric_UGA-AAU |
| | | | | 31_0-1_bulge-asymmetric_-A |
| | | | | 33_5-4_internal_loop-asymmetric_AAGGA-GAAG |
| | | | | 55_1-1_mismatch_C-A |
| 327 | CAGCAGCCACAACTCCCGAAGG | 335 | CAGCAGCCACAACUCCCGAAGGAGCC | -40_1-1_wobble_G-U |
| | AGCCTTTGAAAGTCCTTAATTG | | UUUGAAAGUCCUUAAUUGAAUACAUC | -36_1-1_wobble_U-G |
| | AATACATCCACGGCTATACTTA | | CACGGCUAUACUUAUCCUUUACACCA | -33_1-1_wobble_G-U |
| | TCCTTTACACCACGCTGTTGTTG | | CGCUGUUGUUGAGUGGU | -30_1-1_wobble_G-U |
| | AGTGGT | | | -25_1-1_wobble_U-G |
| | | | | -6_6-6_internal_loop-symmetric_AUUCAU-UACUUA |
| | | | | 0_1-1_mismatch_A-C |
| | | | | 13_3-3_bulge-symmetric_UGA-AAU |
| | | | | 31_0-1_bulge-asymmetric_-A |
| | | | | 33_5-4_internal_loop-asymmetric_AAGGA-GAAG |
| | | | | 55_1-1_mismatch_C-A |
| 328 | CAGCAGCCACAACTCCCGAAGG | 336 | CAGCAGCCACAACUCCCGAAGGAGCC | -40_1-1_wobble_G-U |
| | AGCCTTTGAAAGTCCTTAATTG | | UUUGAAAGUCCUUAAUUGAAUACAUC | -36_1-1_wobble_U-G |
| | AATACATCCACGGTTATACTTA | | CACGGUUAUACUUAUUCUUUGCAUCA | -33_1-1_wobble_G-U |
| | TTCTTTGCATCACGCTGTTGTTG | | CGCUGUUGUUGAGUGGU | -30_1-1_wobble_G-U |
| | AGTGGT | | | -25_1-1_wobble_U-G |
| | | | | -21_1-1_wobble_G-U |
| | | | | -18_1-1_wobble_U-G |
| | | | | -13_1-1_wobble_G-U |
| | | | | -6_6-6_internal_loop-symmetric_AUUCAU-UACUUA |
| | | | | -3_1-1_wobble_G-U |
| | | | | 0_1-1_mismatch_A-C |
| | | | | 13_3-3_bulge-symmetric_UGA-AAU |
| | | | | 31_0-1_bulge-asymmetric_-A |
| | | | | 33_5-4_internal_loop-asymmetric_AAGGA-GAAG |
| | | | | 55_1-1_mismatch_C-A |
| 329 | CTCAGCAGCAGCCACAACTCCG | 337 | CUCAGCAGCAGCCACAACUCCGUGCG | -40_1-1_wobble_G-U |
| | TGCGGGGCCTTTGAAAGTCCTT | | GGGCCUUUGAAAGUCCUUUCAUGAAU | -36_1-1_wobble_U-G |
| | TCATGAATACATCTACGGCTAT | | ACAUCUACGGCUAUACUGGUCCUUUA | -30_1-1_wobble_G-U |
| | ACTGGTCCTTTACACCACACTGT | | CACCACACUGUUGUCGAGUGGU | -6_6-6_internal_loop-symmetric_AUUCAU-UACUGG |
| | TGTCGAGTGGT | | | 0_1-1_mismatch_A-C |
| | | | | 2_1-1_wobble_G-U |
| | | | | 33_2-2_bulge-symmetric_AA-GG |
| | | | | 36_1-1_mismatch_G-G |
| | | | | 38_1-1_mismatch_G-G |
| 330 | CTCAGCAGCAGCCACAACTCCG | 338 | CUCAGCAGCAGCCACAACUCCGUGCG | -40_1-1_wobble_G-U |
| | TGCGGGGCCTTTGAAAGTCCTT | | GGGCCUUUGAAAGUCCUUUCAUGAAU | -36_1-1_wobble_U-G |
| | TCATGAATACATCTACGGCTAT | | ACAUCUACGGCUAUACUGGUCCUUUA | -30_1-1_wobble_G-U |
| | ACTGGTCCTTTACACCGCGCTGT | | CACCGCGCUGUUGUCGAGUGGU | -25_1-1_wobble_U-G |
| | TGTCGAGTGGT | | | -23_1-1_wobble_U-G |
| | | | | -6_6-6_internal_loop-symmetric_AUUCAU-UACUGG |
| | | | | 0_1-1_mismatch_A-C |
| | | | | 2_1-1_wobble_G-U |
| | | | | 33_2-2_bulge-symmetric_AA-GG |
| | | | | 36_1-1_mismatch_G-G |
| | | | | 38_1-1_mismatch_G-G |
| 331 | CTCAGCAGCAGCCACAACTCCG | 339 | CUCAGCAGCAGCCACAACUCCGUGCG | -40_1-1_wobble_G-U |
| | TGCGGGGCCTTTGAAAGTCCTT | | GGGCCUUUGAAAGUCCUUUCAUGAAU | -36_1-1_wobble_U-G |
| | TCATGAATACATCTACGGCTAT | | ACAUCUACGGCUAUACUGGUCCUUUG | -30_1-1_wobble_G-U |
| | ACTGGTCCTTTGCATCGCGCTGT | | CAUCGCGCUGUUGUCGAGUGGU | -25_1-1_wobble_U-G |
| | TGTCGAGTGGT | | | -23_1-1_wobble_U-G |
| | | | | -21_1-1_wobble_G-U |
| | | | | -18_1-1_wobble_U-G |
| | | | | -6_6-6_internal_loop-symmetric_AUUCAU-UACUGG |
| | | | | 0_1-1_mismatch_A-C |
| | | | | 2_1-1_wobble_G-U |
| | | | | 33_2-2_bulge-symmetric_AA-GG |
| | | | | 36_1-1_mismatch_G-G |
| | | | | 38_1-1_mismatch_G-G |
| 332 | CTCAGCAGCAGCCACAACTCCG | 340 | CUCAGCAGCAGCCACAACUCCGUGCG | -40_1-1_wobble_G-U |
| | TGCGGGGCCTTTGAAAGTCCTT | | GGGCCUUUGAAAGUCCUUUCAUGAAU | -36_1-1_wobble_U-G |
| | TCATGAATACGTCTACGGCTAT | | ACGUCUACGGCUAUACUGGUUCUUUG | -30_1-1_wobble_G-U |
| | ACTGGTTCTTTGCATCGCGCTGT | | CAUCGCGCUGUUGUCGAGUGGU | -25_1-1_wobble_U-G |
| | TGTCGAGTGGT | | | -23__wobble_U-G |
| | | | | -21_1-1_wobble_G-U |
| | | | | -18_1-1_wobble_U-G |
| | | | | -13-1-1_wobble_G-U |
| | | | | -6_6-6_internal_loop-symmetric_AUUCAU-UACUGG |
| | | | | 0_1-1_mismatch_A-C |
| | | | | 2_1-1_wobble_G-U |
| | | | | 5_1-1_wobble_U-G |
| | | | | 33_2-2_bulge-symmetric_AA-GG |
| | | | | 36_1-1_mismatch_G-G |
| | | | | 38_1-1_mismatch_G-G |
| 358 | GCCACAACTCCCTCGCGCAACT | 341 | GCCACAACUCCCUCGCGCAACUUUGA | -18_6-6_internal_loop-symmetric_UGGUGU-UGGAGU |
| | TTGAAAGTCCTTTCATGCATAG | | AAGUCCUUUCAUGCAUAGAUCCACGG | -5_7-7_internal_loop-symmetric_AUUCAUU-CCAAAAC |
| | ATCCACGGCTCCAAAACTCCTT | | CUCCAAAACUCCUUUUGGAGUCACUG | 0_1-1_mismatch_A-C |
| | TTGGAGTCACTGTCGTCGAATG | | UCGUCGAAUGGCCACUCCCAGU | 6_1-1_mismatch_G-G |
| | GCCACTCCCAGT | | | 10_1-1_mismatch_U-C |
| | | | | 30_6-6_internal_loop-symmetric_GCCAAG-GCGCAA |
| 359 | GCCACAACTCCCTCGCGCAACT | 342 | GCCACAACUCCCUCGCGCAACUUUGA | -18_6-6_internal_loop-symmetric_UGGUGU-UGGAGU |
| | TTGAAAGTCCTTTCATGCATAG | | AAGUCCUUUCAUGCAUAGAUCCACGG | -5_4-3_bulge-asymmetric_CAUU-CCA |
| | ATCCACGGCTCCAAATTCCTTTT | | CUCCAAAUUCCUUUUGGAGUCACUGU | 0_1-1_mismatch_A-C |
| | GGAGTCACTGTCGTCGAATGGC | | CGUCGAAUGGCCACUCCCAGU | 6_1-1_mismatch_G-G |
| | CACTCCCAGT | | | 10_1-1_mismatch_U-C |
| | | | | 30_6-6_internal_loop-symmetric_GCCAAG-GCGCAA |
| 360 | GCCACAACTCCCTCGCGCAACT | 343 | GCCACAACUCCCUCGCGCAACUUUGA | -18_6-6_internal_loop-symmetric_UGGUGU-UGGAGU |
| | TTGAAAGTCCTTTCATGCATAG | | AAGUCCUUUCAUGCAUAGAUCCACGG | -5_4-4_bulge-symmetric_CAUU-CCAU |
| | ATCCACGGCTCCATAATTCCTTT | | CUCCAUAAUUCCUUUUGGAGUCACUG | 0_1-1_mismatch_A-C |
| | TGGAGTCACTGTCGTCGAATGG | | UCGUCGAAUGGCCACUCCCAGU | 6_1-1_mismatch_G-G |
| | CCACTCCCAGT | | | 10_1-1_mismatch_U-C |
| | | | | 30_6-6_internal_loop-symmetric_GCCAAG-GCGCAA |
| 361 | GCCACAACTCCCTCGCGCAACT | 344 | GCCACAACUCCCUCGCGCAACUUUGA | -18_6-6_internal_loop-symmetric_UGGUGU-UGGAGU |
| | TTGAAAGTCCTTTCATGCATAG | | AAGUCCUUUCAUGCAUAGAUCCACGG | -6_3-3_bulge-symmetric_CAU-CAU |
| | ATCCACGGCTGCATAATTCCTTT | | CUGCAUAAUUCCUUUUGGAGUCACUG | -5_1-1_wobble_U-G |
| | TGGAGTCACTGTCGTCGAATGG | | UCGUCGAAUGGCCACUCCCAGU | 0_1-1_mismatch_A-C |
| | CCACTCCCAGT | | | 6_1-1_mismatch_G-G |
| | | | | 10_1-1_mismatch_U-C |
| | | | | 30_6-6_internal_loop-symmetric_GCCAAG-GCGCAA |
| 362 | GCCACAACTCCCTCCTACAAAC | 345 | GCCACAACUCCCUCCUACAAACUUGA | -10_6-6_internal_loop-symmetric_AGGAAU-CGGAGA |
| | TTGAAAGTCCTTTCATGAATAC | | AAGUCCUUUCAUGAAUACAACCACGG | -6_1-1_mismatch_U-U |
| | AACCACGGCTATTGACGGAGAT | | CUAUUGACGGAGAUUACACCACACUG | 0_1-1_mismatch_A-C |
| | TACACCACACTGTCGTCGAATG | | UCGUCGAAUGGCCACUCCCAGU | 4_1-1_mismatch_A-A |
| | GCCACTCCCAGT | | | 28_6-6_internal_loop-symmetric_AGGCCA-ACAAAC |
| 363 | GCCACAACTCCCTCCTACAAAC | 346 | GCCACAACUCCCUCCUACAAACUUGA | -10_6-6_internal_loop-symmetric_AGGAAU-CGGAGA |
| | TTGAAAGTCCTTTCATGAATAC | | AAGUCCUUUCAUGAAUACAACCACGG | -6_1-2_bulge-asymmetric_U-UU |
| | AACCACGGCTATTTGACGGAGA | | CUAUUUGACGGAGAUUACACCACACU | 0_1-1_mismatch_A-C |
| | TTACACCACACTGTCGTCGAAT | | GUCGUCGAAUGGCCACUCCCAGU | 4_1-1_mismatch_A-A |
| | GGCCACTCCCAGT | | | 28_6-6_internal_loop-symmetric_AGGCCA-ACAAAC |
| 364 | GCCACAACTCCCTCCTACAAAC | 347 | GCCACAACUCCCUCCUACAAACUUGA | -10_6-6_internal_loop-symmetric_AGGAAU-CGGAGA |
| | TTGAAAGTCCTTTCATGAATAC | | AAGUCCUUUCAUGAAUACAACCACGG | -5_2-1_bulge-asymmetric_UU-U |
| | AACCACGGCTTTGACGGAGATT | | CUUUGACGGAGAUUACACCACACUGU | 0_1-1_mismatch_A-C |
| | ACACCACACTGTCGTCGAATGG | | CGUCGAAUGGCCACUCCCAGU | 4_1-1_mismatch_A-A |
| | CCACTCCCAGT | | | 28_6-6_internal loop-symmetric_AGGCCA-ACAAAC |
| 365 | GCCACAACTCCCTCCTATAGAG | 348 | GCCACAACUCCCUCCUAUAGAGUUGA | -6_8-7_internal_loop-asymmetric_GAAUUCAU- |
| | TTGAAAGTCCTTTCATGAATAC | | AAGUCCUUUCAUGAAUACAUCUACGG | UCGCCGA |
| | ATCTACGGCTATCGCCGACTTT | | CUAUCGCCGACUUUACACCACACUGU | 0_1-1_mismatch_A-C |
| | ACACCACACTGTCGTCGAATGG | | CGUCGAAUGGCCACUCCCAGU | 2_1-1_wobble_G-U |
| | CCACTCCCAGT | | | 28_6-6_internal_loop-symmetric_AGGCCA-AUAGAG |
| 366 | GCCACAACTCCCTCCTATAGAG | 349 | GCCACAACUCCCUCCUAUAGAGUUGA | -5_9-8_internal_loop-asymmetric_GAAUUCAUU- |
| | TTGAAAGTCCTTTCATGAATAC | | AAGUCCUUUCAUGAAUACAUCUACGG | CUCGCCGA |
| | ATCTACGGCTCTCGCCGACTTTA | | CUCUCGCCGACUUUACACCACACUGU | 0_1-1_mismatch_A-C |
| | CACCACACTGTCGTCGAATGGC | | CGUCGAAUGGCCACUCCCAGU | 2_1-1_wobble_G-U |
| | CACTCCCAGT | | | 28_6-6_internal_loop-symmetric_AGGCCA-AUAGAG |
| 367 | GCCACAACTCCCTCCTATAGCG | 350 | GCCACAACUCCCUCCUAUAGCGUUGA | -5_9-8_internal_loop-asymmetric_GAAUUCAUU- |
| | TTGAAAGTCCTTTCATGAATAC | | AAGUCCUUUCAUGAAUACAUCUACGG | CUCGCCGA |
| | ATCTACGGCTCTCGCCGACTTTA | | CUCUCGCCGACUUUACACCACACUGU | 0_1-1_mismatch_A-C |
| | CACCACACTGTCGTCGAATGGC | | CGUCGAAUGGCCACUCCCAGU | 2_1-1_wobble_G-U |
| | CACTCCCAGT | | | 28_2-1_bulge-asymmetric_AG-G |
| | | | | 32_2-3_bulge-asymmetric_CA-AUA |
| 368 | GCCACAACTCCCTCCTTGACGG | 351 | GCCACAACUCCCUCCUUGACGGCGGA | -16_6-6_internal_loop-symmetric_GUGUAA-ACCGUG |
| | CGGAAAGTCCTTTCATGGCACA | | AAGUCCUUUCAUGGCACAUCCACGGC | -6_3-2_bulge-asymmetric_CAU-CC |
| | TCCACGGCTACCAATTCCTACC | | UACCAAUUCCUACCGUGCACACUGUC | 0_1-1_mismatch_A-C |
| | GTGCACACTGTCGTCGAATGGC | | GUCGAAUGGCCACUCCCAGU | 8_2-1_bulge-asymmetric_AU-C |
| | CACTCCCAGT | | | 10_1-1_wobble_U-G |
| | | | | 26_4-4_bulge-symmetric_AAAG-GGCG |
| | | | | 31_1-1_mismatch_C-A |
| 369 | GCCACAACTCCCTCCTTGATGG | 352 | GCCACAACUCCCUCCUUGAUGGCGGA | -16_6-6_internal_loop-symmetric_GUGUAA-ACCGUG |
| | CGGAAAGTCCTTTCATGGCACA | | AAGUCCUUUCAUGGCACAUCCACGGC | -6_3-2_bulge-asymmetric_CAU-CC |
| | TCCACGGCTACCAATTCCTACC | | UACCAAUUCCUACCGUGCACACUGUC | 0_1-1_mismatch_A-C |
| | GTGCACACTGTCGTCGAATGGC | | GUCGAAUGGCCACUCCCAGU | 8_2-1_bulge-asymmetric_AU-C |
| | CACTCCCAGT | | | 10_1-1_wobble_U-G |
| | | | | 26_6-6_internal_loop-symmetric_AAAGGC-AUGGCG |
| 370 | GCCACAACTCCCTCCTTGATGG | 353 | GCCACAACUCCCUCCUUGAUGGCGGA | -16_6-6_internal_loop-symmetric_GUGUAA-ACCGUG |
| | CGGAAAGTCCTTTCATGGCACA | | AAGUCCUUUCAUGGCACAUCCACGGC | -6_3-2_bulge-asymmetric_CAU-UC |
| | TCCACGGCTATCAATTCCTACC | | UAUCAAUUCCUACCGUGCACACUGUC | 0_1-1_mismatch_A-C |
| | GTGCACACTGTCGTCGAATGGC | | GUCGAAUGGCCACUCCCAGU | 8_2-1_bulge-asymmetric_AU-C |
| | CACTCCCAGT | | | 10_1-1_wobble_U-G |
| | | | | 26_6-6_internal_loop-symmetric_AAAGGC-AUGGCG |
| 371 | GCCACAACTCCCTCCTTGTAGG | 354 | GCCACAACUCCCUCCUUGUAGGAGGA | -16_6-6_internal_loop-symmetric_GUGUAA-CCUCUA |
| | AGGAAAGTCCTTTCATTAATAC | | AAGUCCUUUCAUUAAUACAUCCACGG | -6_2-2_bulge-symmetric_AU-UG |
| | ATCCACGGCAATGGAATTCCTC | | CAAUGGAAUUCCUCCUCUACACACUG | -4_1-1_mismatch_A-A |
| | CTCTACACACTGTCGTCGAATG | | UCGUCGAAUGGCCACUCCCAGU | 0_1-1_mismatch_A-C |
| | GCCACTCCCAGT | | | 11_1-1_mismatch_C-U |
| | | | | 26_6-6_internal_loop-symmetric_AAAGGC-UAGGAG |
| 372 | GCCACAACTCCCTCCTTGTAGG | 355 | GCCACAACUCCCUCCUUGUAGGAGGA | -16_6-6_internal_loop-symmetric_GUGUAA-CCUCUA |
| | AGGAAAGTCCTTTCATTAATAC | | AAGUCCUUUCAUUAAUACAUCCACGG | -4_4-5_internal_loop-asymmetric_AUUA-ACUUG_0_1- |
| | ATCCACGGCACTTGGAATTCCT | | CACUUGGAAUUCCUCCUCUACACACU | 1_mismatch_A-C |
| | CCTCTACACACTGTCGTCGAAT | | GUCGUCGAAUGGCCACUCCCAGU | 11_1-1_mismatch_C-U |
| | GGCCACTCCCAGT | | | 26_6-6_internal_loop-symmetric_AAAGGC-UAGGAG |
| 373 | GCCACAACTCCCTCCTTGTAGG | 356 | GCCACAACUCCCUCCUUGUAGGAGGA | -16_6-6_internal_loop-symmetric_GUGUAA-CCUCUA |
| | AGGAAAGTCCTTTCATTAATAC | | AAGUCCUUUCAUUAAUACAUCCACGG | -6_2-2_bulge-symmetric_AU-UG |
| | ATCCACGGCTATGGAATTCCTC | | CUAUGGAAUUCCUCCUCUACACACUG | 0_1-1_mismatch_A-C |
| | CTCTACACACTGTCGTCGAATG | | UCGUCGAAUGGCCACUCCCAGU | 11_1-1_mismatch_C-U |
| | GCCACTCCCAGT | | | 26_6-6_internal_loop-symmetric_AAAGGC-UAGGAG |
| 374 | GCCACAACTCCCTCCTTGTAGG | 357 | GCCACAACUCCCUCCUUGUAGGAGGA | -16_6-6_internal_loop-symmetric_GUGUAA-CCUCUA |
| | AGGAAAGTCCTTTCATTAATAC | | AAGUCCUUUCAUUAAUACAUCCACGG | -5_3-4_bulge-asymmetric_AUU-CUUG |
| | ATCCACGGCTCTTGGAATTCCTC | | CUCUUGGAAUUCCUCCUCUACACACU | 0_1-1_mismatch_A-C |
| | CTCTACACACTGTCGTCGAATG | | GUCGUCGAAUGGCCACUCCCAGU | 11_1-1_mismatch_C-U |
| | GCCACTCCCAGT | | | 26_6-6_internal_loop-symmetric_AAAGGC-UAGGAG |
| 378 | CTCAGCAGCAGCCACAACTCCC | 380 | CUCAGCAGCAGCCACAACUCCCUCCU | -14_6-6_internal_loop-symmetric_GUAAAG-GCGCUG |
| | TCCTTGTAAGGAGAAAGTCCTT | | UGUAAGGAGAAAGUCCUUUCAUGAA | -4_3-3_bulge-symmetric_UUA-GGC |
| | TCATGAATACATCCACGGCGGC | | UACAUCCACGGCGGCUGAAUUCGCGC | 0_1-1_mismatch_A-C |
| | TGAATTCGCGCTGACCACACTG | | UGACCACACUGUCGUCGAAUGGC | 26_6-6_internal_loop-symmetric_AAAGGC-UAAGGA |
| | TCGTCGAATGGC | | | |
| 379 | CTCAGCAGCAGCCACAACTCCC | 381 | CUCAGCAGCAGCCACAACUCCCUCCU | -10_6-6_internal_loop-symmetric_AGGAAU-UAGAGC |
| | TCCTCTCGAGTTGAAAGTCCTTT | | CUCGAGUUGAAAGUCCUUUCAUGAAU | -6_2-2_bulge-symmetric_AU-CG |
| | CATGAATACATCCACGGCTACG | | ACAUCCACGGCUACGGAUAGAGCUUA | 0_1-1_mismatch_A-C |
| | GATAGAGCTTACACCACACTGT | | CACCACACUGUCGUCGAAUGGC | 28_6-6_internal_loop-symmetric_AGGCCA-CUCGAG |
| | CGTCGAATGGC | | | |

### Example 3

### Identification of Parental Guide RNA Architectures and Sequences That Target SNCA Exon 2 TIS

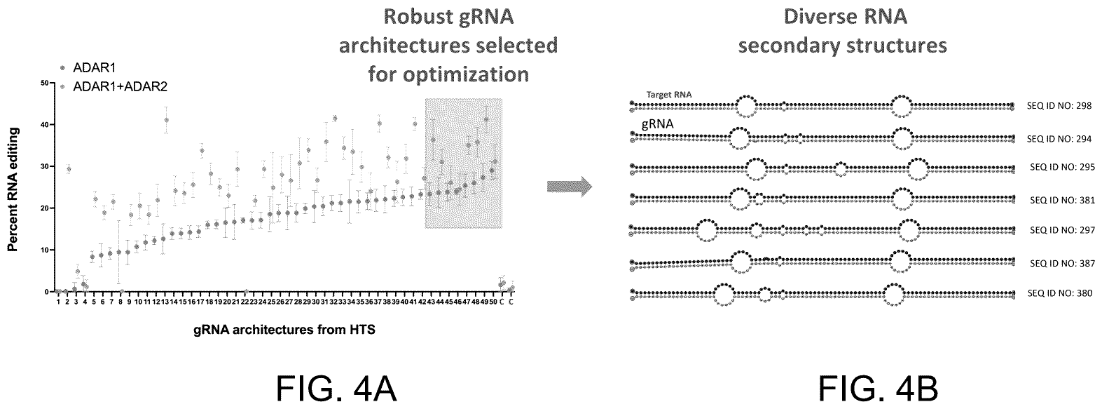

Cell-free high throughput screening of over 40,000 structurally distinct gRNAs was performed. The high throughput screen quantified the effect of varying secondary structure on ADAR-mediated RNA editing of SNCA. The engineered guide RNAs recited in Table 4 below were selected as parental guide RNAs as starting scaffolds to promote ADAR recruitment, facilitate editing of the SNCA target RNA, and reduce alpha-synuclein protein expression (FIGS. 4A-4B). Each guide RNA recited in Table 4 and shown in FIG. 4B exhibited greater than 20% RNA editing via endogenous ADAR1 and endogenous ADAR1+overexpressed ADAR2.

TABLE 4
Guide RNA Sequences Targeting SNCA TIS.

| SEQ ID | | |
|---|---|---|
| NOS: | Sequence | Structural Features |
| 294 | AGCAGCCACAACUCCCUCC | -10_6-6_internal_loop-symmetric_AGGAAU-CGGAGA |
| | UACAAACUUGAAAGUCCU | -6_0-1_bulge-asymmetric_-U |
| | UUCAUGAAUACAACCACG | 0_1-1_mismatch_A-C |
| | GCUAAUUGACGGAGAUUA | 4_1-1_mismatch_A-A |
| | CACCACACUGUC | 28_6-6_internal_loop-symmetric_AGGCCA-ACAAAC |
| 295 | CAGCAGCCACAACUCCCAG | -6_6-6_internal_loop-symmetric_AUUCAU-CGACAG |
| | GAUCGCCUUUGAAAGUCC | 0_1-1_mismatch_A-C |
| | UUCCCUGAAUACAUCCACG | 13_1-1_mismatch_U-C |
| | GCUACGACAGUCCUUUAC | 15_1-1_mismatch_A-C |
| | ACCACACUGUCGUCGAAU | 32_6-6_internal_loop-symmetric_CAAGGA-AGGAUC |
| | GGC | |
| 297 | CAGCAGCCACAACUCCCUC | -18_6-6_internal_loop-symmetric_UGGUGU-UGGAGU |
| | GCGCAACUUUGAAAGUCC | -6_3-3_bulge-symmetric_CAU-CAU |
| | UUUCAUGCAUAGAUCCAC | 0_1-1_mismatch_A-C |
| | GGCUACAUAAUUCCUUUU | 6_1-1_mismatch_G-G |
| | GGAGUCACUGUCGUCGA | 10_1-1_mismatch_U-C |
| | | 30_6-6_internal_loop-symmetric_GCCAAG-GCGCAA |
| 298 | CUCAGCAGCAGCCACAACU | -6_6-6_internal_loop-symmetric_AUUCAU-CAUAUC |
| | CCAAUGAAGGCCUUUGAA | 0_1-1_mismatch_A-C |
| | AGUCCUUUCAUGAAUACA | 2_1-1_wobble_G-U |
| | UCUACGGCUACAUAUCUCC | 33_6-6_internal_loop-symmetric_AAGGAG-AAUGAA |
| | UUUACACCACACUGUCGUC | |
| | GAAUGGC | |
| 299 | CUCAGCAGCAGCCACAACU | -6_6-6_internal_loop-symmetric_AUUCAU-CAUAUC |
| | CCAGAAUAGGCCUUUGAA | 0_1-1_mismatch_A-C |
| | AGUCCUUUCAUGAAUAAC | 2_1-1_wobble_G-U |
| | UCCACGGCUAUAAAUCUCC | 33_6-6_internal_loop-symmetric_AAGGAG-AAUGAA |
| | UUUACACCACACUGUCGUC | |
| | GAAUGGC | |
| 380 | CUCAGCAGCAGCCACAACU | -14_6-6_internal_loop-symmetric_GUAAAG-GCGCUG |
| | CCCUCCUUGUAAGGAGAA | -4_3-3_bulge-symmetric_UUA-GGC |
| | AGUCCUUUCAUGAAUACA | 0_1-1_mismatch_A-C |
| | UCCACGGCGGCUGAAUUC | 26_6-6_internal_loop-symmetric_AAAGGC-UAAGGA |
| | GCGCUGACCACACUGUCGU | |
| | CGAAUGGC | |
| 381 | CUCAGCAGCAGCCACAACU | -10_6-6_internal_loop-symmetric_AGGAAU-UAGAGC |
| | CCCUCCUCUCGAGUUGAAA | -6_2-2_bulge-symmetric_AU-CG |
| | GUCCUUUCAUGAAUACAU | 0_1-1_mismatch_A-C |
| | CCACGGCUACGGAUAGAG | 28_6-6_internal_loop-symmetric_AGGCCA-CUCGAG |
| | CUUACACCACACUGUCGUC | |
| | GAAUGGC | |

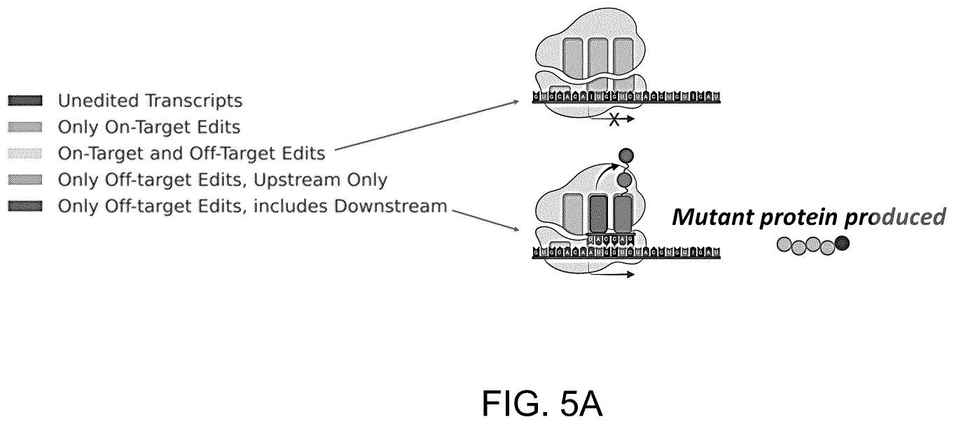

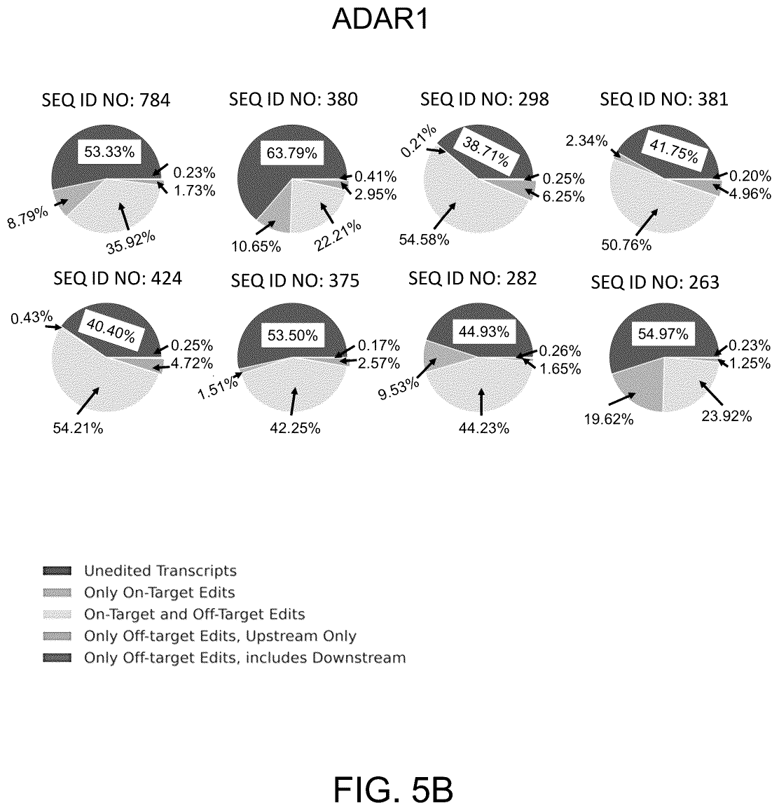

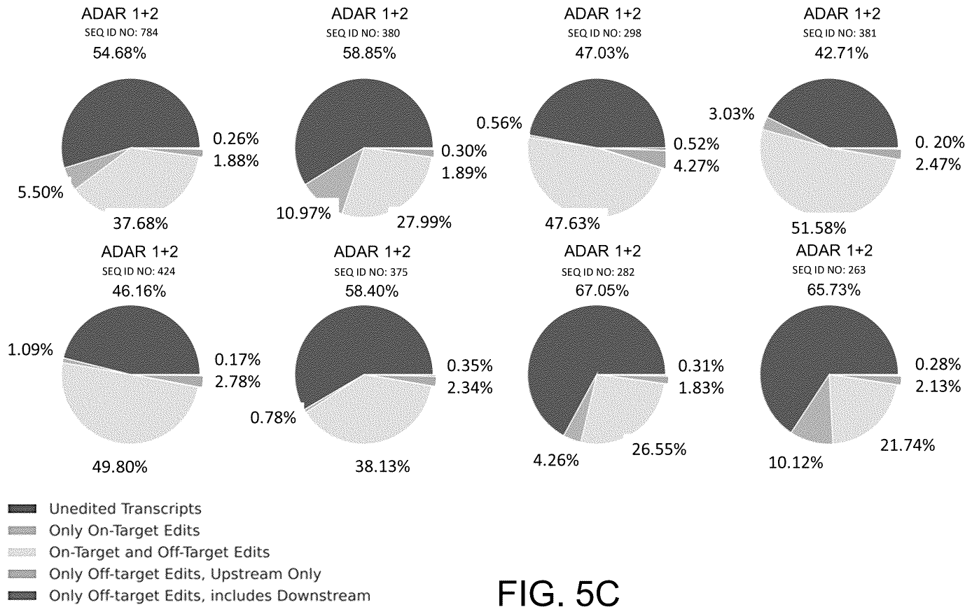

Next, the amount of on-target and off-target editing for each of the 8 engineered gRNAs in Table 4 via endogenous ADAR1 or ADAR 1 with ADAR2 was determined. A targeting specificity assay was performed with the guide RNAs and endogenous ADAR1 or ADAR 1 with ADAR2. RNA editing was analyzed by next generation sequencing (NGS) to identify unedited transcripts, on target edits, on and off target edits, off target edits upstream of the codon 1 TIS (5′ region) and off-target edits downstream of the codon 1 TIS (3′) (FIG. 5A). Single RNA molecules were analyzed and the results are provided in Tables 5 and 6 and FIGS. 5B-SC.

TABLE 5
ADAR 1 On-Target and Off-Target Editing.

| | | | | On- | Only off- | |
|---|---|---|---|---|---|---|
| | | | | Target | target | |
| | | | Only On- | and Off- | edits, | Only off- |
| | | Unedited | Target | Target | upstream | target edits, |
| SEQ ID | | Transcripts | Edits | edits | only | downstream |
| NO: | Sequence | % | % | % | % | % |
| 298 | CUCAGCAGCAGCC | 38.71 | 0.21 | 54.58 | 6.25 | 0.25 |
| | ACAACUCCAAUGA | | | | | |
| | AGGCCUUUGAAAG | | | | | |
| | UCCUUUCAUGAAU | | | | | |
| | ACAUCUACGGCUA | | | | | |
| | CAUAUCUCCUUUA | | | | | |
| | CACCACACUGUCG | | | | | |
| | UCGAAUGGC | | | | | |
| 294 | AGCAGCCACAACU | 40.40 | 0.43 | 54.21 | 4.72 | 0.25 |
| | CCCUCCUACAAAC | | | | | |
| | UUGAAAGUCCUUU | | | | | |
| | CAUGAAUACAACC | | | | | |
| | ACGGCUAAUUGAC | | | | | |
| | GGAGAUUACACCA | | | | | |
| | CACUGUC | | | | | |
| 295 | CAGCAGCCACAAC | 44.93 | 9.53 | 44.23 | 1.05 | 0.26 |
| | UCCCAGGAUCGCC | | | | | |
| | UUUGAAAGUCCUU | | | | | |
| | CCCUGAAUACAUC | | | | | |
| | CACGGCUACGACA | | | | | |
| | GUCCUUUACACCA | | | | | |
| | CACUGUCGUCGAA | | | | | |
| | UGGC | | | | | |
| 299 | CUCAGCAGCAGCC | 53.33 | 8.79 | 35.92 | 1.73 | 0.23 |
| | ACAACUCCAGAAU | | | | | |
| | AGGCCUUUGAAAG | | | | | |
| | UCCUUUCAUGAAU | | | | | |
| | AACUCCACGGCUA | | | | | |
| | UAAAUCUCCUUUA | | | | | |
| | CACCACACUGUCG | | | | | |
| | UCGAAUGGC | | | | | |
| 297 | CAGCAGCCACAAC | 53.50 | 1.51 | 42.25 | 2.57 | 0.17 |
| | UCCCUCGCGCAAC | | | | | |
| | UUUGAAAGUCCUU | | | | | |
| | UCAUGCAUAGAUC | | | | | |
| | CACGGCUACAUAA | | | | | |
| | UUCCUUUUGGAGU | | | | | |
| | CACUGUCGUCGA | | | | | |
| 263 | CUCAGCAGCAGCC | 54.97 | 19.62 | 23.92 | 1.25 | 0.23 |
| | ACAACUCCCUCCU | | | | | |
| | GAAUGAUUGAAAG | | | | | |
| | UCCUUUCAUGAAU | | | | | |
| | ACAUCCACGGCAU | | | | | |
| | GAUCUGAGUUACA | | | | | |
| | CCACACUGUCGUC | | | | | |
| | GAAUGGC | | | | | |
| 380 | CUCAGCAGCAGCC | 63.79 | 10.65 | 22.21 | 2.95 | 0.41 |
| | ACAACUCCCUCCU | | | | | |
| | UGUAAGGAGAAAG | | | | | |
| | UCCUUUCAUGAAU | | | | | |
| | ACAUCCACGGCGG | | | | | |
| | CUGAAUUCGCGCU | | | | | |
| | GACCACACUGUCG | | | | | |
| | UCGAAUGGC | | | | | |
| 381 | CUCAGCAGCAGCC | 41.75 | 2.34 | 50.76 | 4.96 | 0.20 |
| | ACAACUCCCUCCUC | | | | | |
| | UCGAGUUGAAAGU | | | | | |
| | CCUUUCAUGAAUA | | | | | |
| | CAUCCACGGCUAC | | | | | |
| | GGAUAGAGCUUAC | | | | | |
| | ACCACACUGUCGU | | | | | |
| | CGAAUGGC | | | | | |

TABLE 6
ADAR 1 + ADAR 2 On-Target and Off-target Editing.

| | | | | On- | Only off- | |
|---|---|---|---|---|---|---|
| | | | | Target | target | Only off- |
| | | | Only On- | and Off- | edits, | target |
| | | Unedited | Target | Target | upstream | edits, |
| SEQ ID | | Transcripts | Edits | Edits | only | downstream |
| NO: | Sequence | % | % | % | % | % |
| 298 | CUCAGCAGCAGCCA | 47.03 | 0.56 | 47.63 | 4.27 | 0.52 |
| | CAACUCCAAUGAAG | | | | | |
| | GCCUUUGAAAGUCC | | | | | |
| | UUUCAUGAAUACAU | | | | | |
| | CUACGGCUACAUAU | | | | | |
| | CUCCUUUACACCAC | | | | | |
| | ACUGUCGUCGAAUG | | | | | |
| | GC | | | | | |
| 294 | AGCAGCCACAACUC | 46.16 | 1.09 | 49.80 | 2.78 | 0.17 |
| | CCUCCUACAAACUU | | | | | |
| | GAAAGUCCUUUCAU | | | | | |
| | GAAUACAACCACGG | | | | | |
| | CUAAUUGACGGAGA | | | | | |
| | UUACACCACACUGU | | | | | |
| | C | | | | | |
| 295 | CAGCAGCCACAACU | 67.05 | 4.26 | 26.55 | 1.83 | 0.31 |
| | CCCAGGAUCGCCUU | | | | | |
| | UGAAAGUCCUUCCC | | | | | |
| | UGAAUACAUCCACG | | | | | |
| | GCUACGACAGUCCU | | | | | |
| | UUACACCACACUGU | | | | | |
| | CGUCGAAUGGC | | | | | |
| 299 | CUCAGCAGCAGCCA | 54.68 | 5.50 | 37.68 | 1.88 | 0.26 |
| | CAACUCCAGAAUAG | | | | | |
| | GCCUUUGAAAGUCC | | | | | |
| | UUUCAUGAAUAACU | | | | | |
| | CCACGGCUAUAAAU | | | | | |
| | CUCCUUUACACCAC | | | | | |
| | ACUGUCGUCGAAUG | | | | | |
| | GC | | | | | |
| 297 | CAGCAGCCACAACU | 58.40 | 0.78 | 38.13 | 2.34 | 0.35 |
| | CCCUCGCGCAACUU | | | | | |
| | UGAAAGUCCUUUCA | | | | | |
| | UGCAUAGAUCCACG | | | | | |
| | GCUACAUAAUUCCU | | | | | |
| | UUUGGAGUCACUGU | | | | | |
| | CGUCGA | | | | | |
| 263 | CUCAGCAGCAGCCA | 65.73 | 10.12 | 21.74 | 2.13 | 0.28 |
| | CAACUCCCUCCUGA | | | | | |
| | AUGAUUGAAAGUCC | | | | | |
| | UUUCAUGAAUACAU | | | | | |
| | CCACGGCAUGAUCU | | | | | |
| | GAGUUACACCACAC | | | | | |
| | UGUCGUCGAAUGGC | | | | | |
| 380 | CUCAGCAGCAGCCA | 58.85 | 10.97 | 27.99 | 1.89 | 0.30 |
| | CAACUCCCUCCUUG | | | | | |
| | UAAGGAGAAAGUCC | | | | | |
| | UUUCAUGAAUACAU | | | | | |
| | CCACGGCGGCUGAA | | | | | |
| | UUCGCGCUGACCAC | | | | | |
| | ACUGUCGUCGAAUG | | | | | |
| | GC | | | | | |
| 381 | CUCAGCAGCAGCCA | 42.71 | 3.03 | 51.58 | 2.47 | 0.20 |
| | CAACUCCCUCCUCU | | | | | |
| | CGAGUUGAAAGUCC | | | | | |
| | UUUCAUGAAUACAU | | | | | |
| | CCACGGCUACGGAU | | | | | |
| | AGAGCUUACACCAC | | | | | |
| | ACUGUCGUCGAAUG | | | | | |
| | GC | | | | | |

As shown in Table 5 and Table 6, the eight guide RNAs recited in Table 4 produced edits via ADAR1 and ADAR1+ADAR2 that were mostly either on-target (i.e., editing of the adenosine of Exon 2 Codon 1 TIS) or a combination of on-target and off-target. In contrast, few transcripts were present having only off-target edits. As discussed above with respect to Example 1, editing of the Exon 2 Codon 1 TIS results in significant reduction of alpha-synuclein protein. Further, as shown in FIG. 5A, it is expected that only those mRNA transcripts having off-target downstream edits alone could result in production of mutant alpha-synuclein variants (rather than a reduction in wild-type alpha-synuclein levels. Computational analysis enabled rapid identification and enrichment of structures efficient editing SNCA. Accordingly, these data suggest that the guide RNAs recited in Table 4 are more likely to produce a reduction of wild-type alpha-synuclein levels rather than production of alpha-synuclein variants.

### Example 4

### Engineering of SNCA-Translation Initiation Site (TIS) Guide RNA Macrofootprint

High-throughput screening (HTS) was performed to identify and fine-tune macrofootprints of the gRNAs with optimal positioning at the target site, barbell coordinates, and length. Guide RNAs from cell-free HTS were translated in-vitro, in cellular models of endogenous ADAR and native SNCA. HEK293 cells expressing endogenous ADAR1 or endogenous ADAR1 plus recombinant ADAR2 were transiently transfected with plasmid DNA encoding SNCA TIS targeting guide RNAs. RNA sequences were analyzed for editing efficiency. High performing gRNA architectures were selected from the screen based on efficiency and specificity in ADAR1 and ADAR1+ADAR2 HEK293 cells. Guide RNA structural features were further optimized to ablate editing of off-target adenosines. The goal of the in vitro assay was to determine both on and off target editing in systems that are not limited by gRNA expression. Furthermore, single site integration of guide RNAs in HEK cells were used to rank on-target editing in gRNA expression systems.

In order to select the optimal guide length and barbell positions of each guide RNA, guide RNAs having the SEQ ID NO: 298, SEQ ID NO: 297, and SEQ ID NO: 296 architecture were tested by stable integration into HEK293T cells using a single-integration expression system that produces a single genomic copy of a particular guide RNA per cell. Single integration assays were performed on the engineered guide RNAs to determine editing efficiency. Guide RNAs were cloned into a plasmid with a puromycin resistance genes to select for HEK293T cells that were transfected with the guide and achieved full integration. Cells were passaged until transient plasmid levels dropped beyond detection. Guide RNAs that proceeded to the next stage of guide development were chosen for higher on-target editing, favorable off-target profiles, and other characteristics to retain diversity within the gRNAs tested.

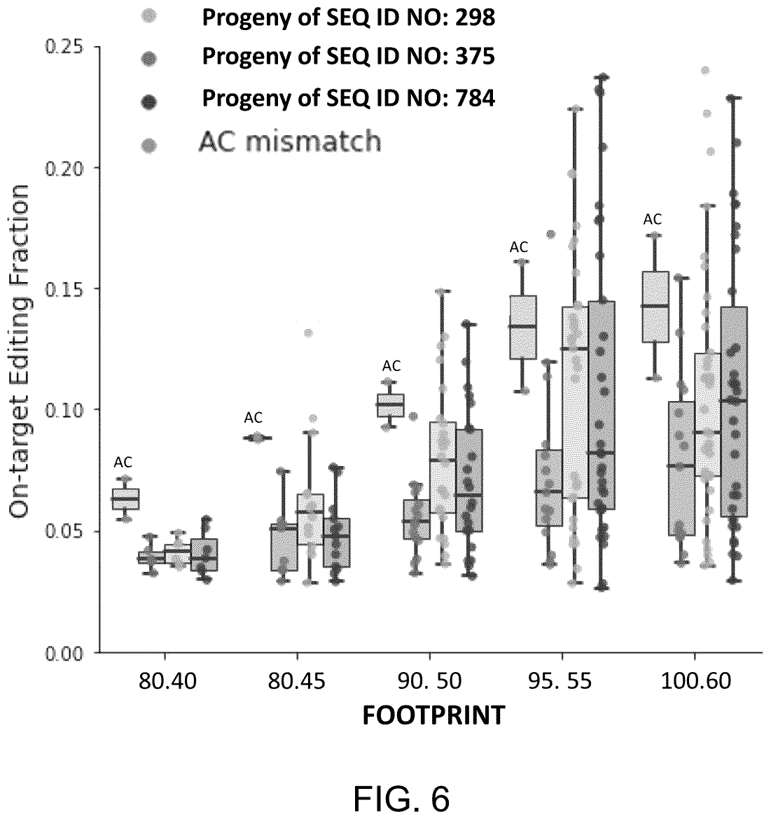

First, the length of the macrofootprint for the guide RNAs having a polynucleotide sequence of SEQ ID NO: 298, SEQ ID NO: 375, and SEQ ID NO: 784 architectures was scanned using the following configurations: 100.60 (guide length: 100, mismatch position: 60), 95.55 (guide length: 95, mismatch position: 55), 90.50 (guide length: 90, mismatch position: 50), 80.45 (guide length: 80, mismatch position: 45), and 80.40 (guide length: 80, mismatch position: 40). Average on-target editing increased with overall guide length (FIG. 6).

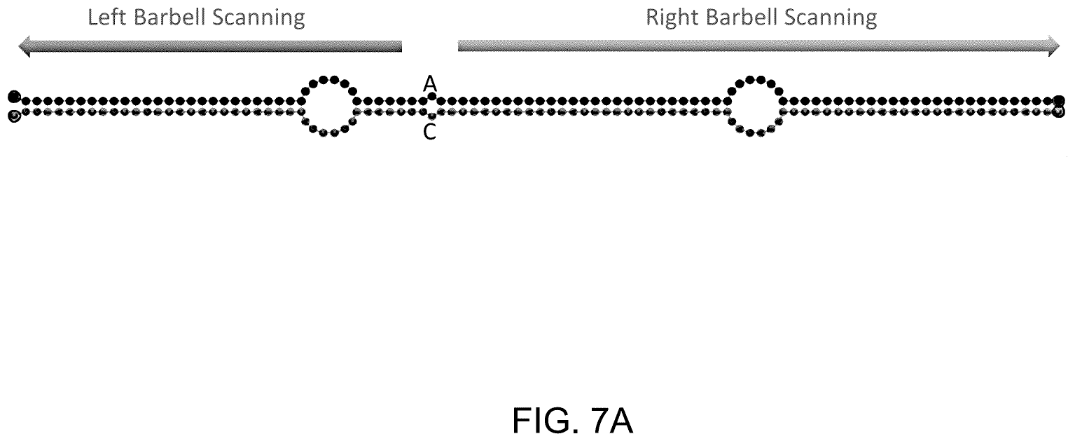

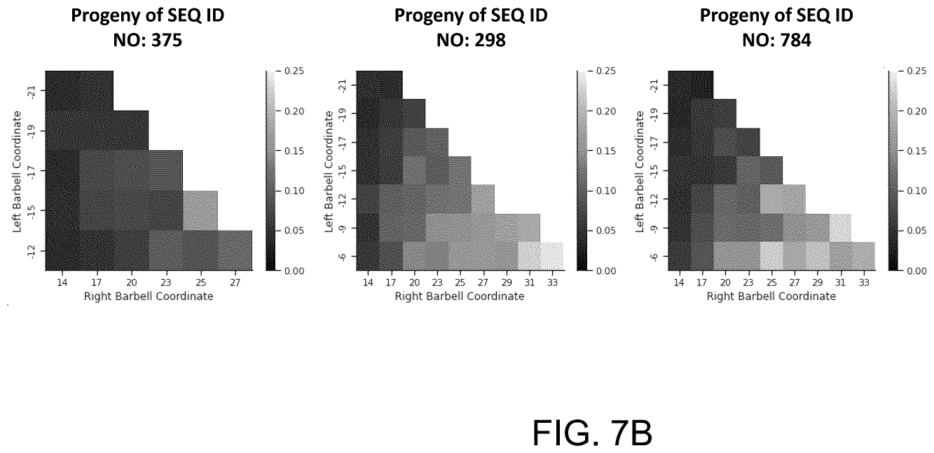

Next, a scan was performed for the optimal position of the left barbell and right barbell for these guides were identified (FIG. 7A). Heatmaps were generated to determine the most favorable positions for left barbell and right barbell positioning on either side of the target mismatch to enhance RNA editing capability for each guide RNA based on the parental guide RNAs having a polynucleotide sequence of SEQ ID NO: 298, SEQ ID NO: 375, or SEQ ID NO: 784 (FIG. 7B).

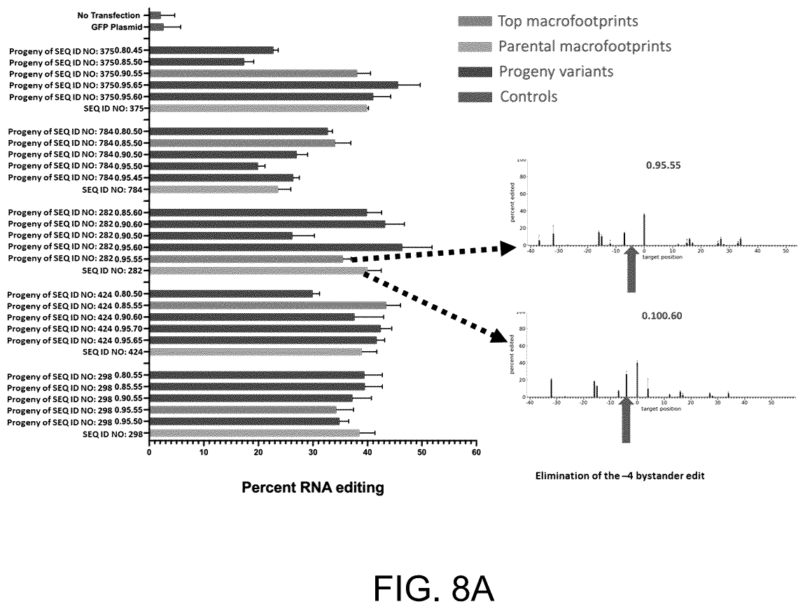

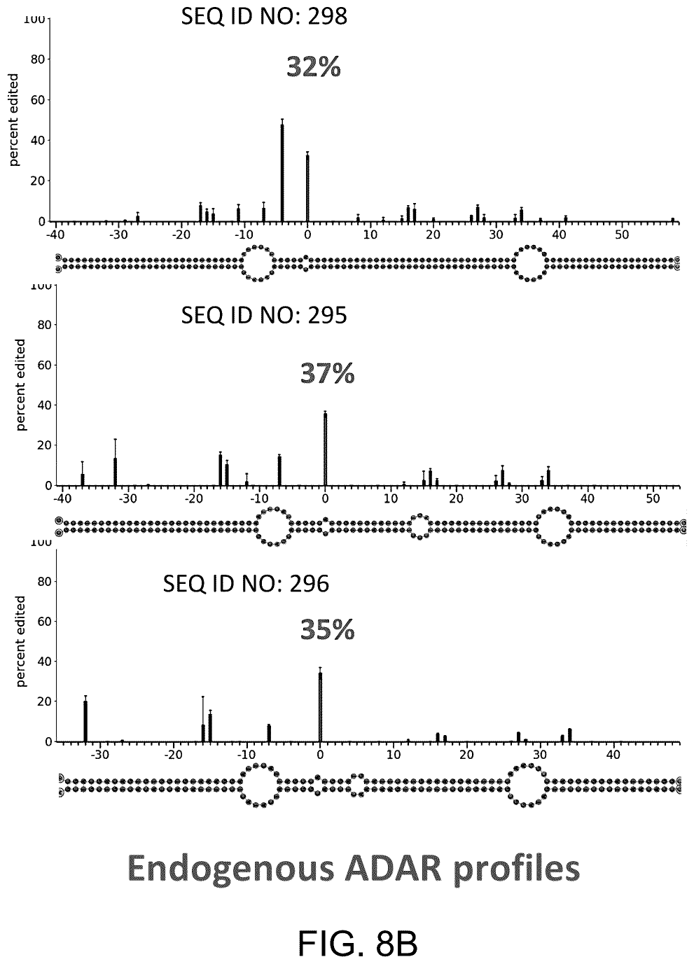

Next, the macrofootprint of guide RNAs with the SEQ ID NO: 298, SEQ ID NO: 297, SEQ ID NO: 296, SEQ ID NO: 295, and SEQ ID NO: 294 architecture was engineered using transient transfection assays. The effect of guide RNA length for guide RNAs having the SEQ ID NO: 298, SEQ ID NO: 297, and SEQ ID NO: 296 architectures on ADAR editing efficiency was determined as discussed above with the single integration assay. FIG. 8A shows the effect of guide RNA length on editing efficiency for select guide RNAs via ADAR. As shown in FIG. 8B, select guide RNAs such as SEQ ID NO: 296 have significantly-reduced bystander editing of the adenosine at position −4, relative to the adenosine of the Codon 1 TIS. Table 7 below shows a summary of optimal guide RNA architectures from the transient transfection assay and single integration assay.

TABLE 7
SNCA gRNA Architectures

| | | | | | Non- |
|---|---|---|---|---|---|
| | | | | | coding -4 |
| | | Macro- | | | off-target |
| SEQ ID | | footprint. | Barbell | | editing |
| NO: | Sequence | length | Coordinates | Assay | (yes/no) |
| 293 | CAGCAGCCACAACUCCCUC | 95.55 | −8, +28 | Transient | YES |
| | CUAUAGAGUUGAAAGUCC | | | Transfection | |
| | UUUCAUGAAUACAUCUAC | | | Assay | |
| | GGCUAAUCGCCGACUUUA | | | | |
| | CACCACACUGUCGUCGAA | | | | |
| | UGGC | | | | |
| 294 | AGCAGCCACAACUCCCUCC | 85.55 | −10, +28 | Transient | YES |
| | UACAAACUUGAAAGUCCU | | | Transfection | |
| | UUCAUGAAUACAACCACG | | | Assay | |
| | GCUAAUUGACGGAGAUUA | | | | |
| | CACCACACUGUC | | | | |
| 295 | CAGCAGCCACAACUCCCAG | 95.55 | −6, +32 | Transient | NO |
| | GAUCGCCUUUGAAAGUCC | | | Transfection | |
| | UUCCCUGAAUACAUCCAC | | | Assay | |
| | GGCUACGACAGUCCUUUA | | | | |
| | CACCACACUGUCGUCGAA | | | | |
| | UGGC | | | | |
| 299 | CUCAGCAGCAGCCACAACU | 85.50 | −6, +26 | Transient | NO |
| | CCAGAAUAGGCCUUUGAA | | | Transfection | |
| | AGUCCUUUCAUGAAUAAC | | | Assay | |
| | UCCACGGCUAUAAAUCUC | | | | |
| | CUUUACACCACACUGUCG | | | | |
| | UCGAAUGGC | | | | |
| 297 | CAGCAGCCACAACUCCCUC | 90.55 | −17, +30 | Transient | NO |
| | GCGCAACUUUGAAAGUCC | | | Transfection | |
| | UUUCAUGCAUAGAUCCAC | | | Assay | |
| | GGCUACAUAAUUCCUUUU | | | | |
| | GGAGUCACUGUCGUCGA | | | | |
| 298 | CUCAGCAGCAGCCACAACU | 100.60 | −6, +33 | Single | YES |
| | CCAAUGAAGGCCUUUGAA | | | Integration | |
| | AGUCCUUUCAUGAAUACA | | | | |
| | UCUACGGCUACAUAUCUC | | | | |
| | CUUUACACCACACUGUCG | | | | |
| | UCGAAUGGC | | | | |
| 299 | CUCAGCAGCAGCCACAACU | 100.60 | −6, +33 | Single | NO |
| | CCAGAAUAGGCCUUUGAA | | | Integration | |
| | AGUCCUUUCAUGAAUAAC | | | | |
| | UCCACGGCUAUAAAUCUC | | | | |
| | CUUUACACCACACUGUCG | | | | |
| | UCGAAUGGC | | | | |

AAV-packaged SNCA-TIS targeting guide RNAs and functional validation in-vitro and in-vivo

SNCA-targeting guide RNAs
SEQ ID NO: 375
(CUCAGCAGCAGCCACAACUCCCUCGCGCAACUUUGAAAGUCCUUUCAUGCAUA
GAUCCACGGCUACAUAAUUCCUUUUGGAGUCACUGUCGUCGAAUGGC);
written as DNA-
SEQ ID NO: 748
(CTCAGCAGCAGCCACAACTCCCTCGCGCAACTTTGAAAGTCCTTTCATGCATAG
ATCCACGGCTACATAATTCCTTTTGGAGTCACTGTCGTCGAATGGC);
SEQ ID NO: 376
(CUCAGCAGCAGCCACAACUCCCUCCUAUAGAGUUGAAAGUCCUUUCAUGAAU
ACAUCUACGGCUAAUCGCCGACUUUACACCACACUGUCGUCGAAUGGC);
written as DNA-
SEQ ID NO: 749
(CTCAGCAGCAGCCACAACTCCCTCCTATAGAGTTGAAAGTCCTTTCATGAATAC
ATCTACGGCTAATCGCCGACTTTACACCACACTGTCGTCGAATGGC

selected in Example 4 were packaged into AAV vectors with regulatory element variants reg1, reg2, reg3, and reg4. The AAV vectors were delivered to mouse primary neurons in-vitro to demonstrate: (1) that the guide RNAs are able to facilitate editing of SNCA mRNA in the cell, and (b) that the guide RNAs reduce the level of wild-type alpha-synuclein in the cell. The dual guide RNA vectors comprise two guide RNAs. To produce the AAV vectors, a producer cell line is transfected with: (1) DNA necessary for AAV replication and synthesis of an AAV capsid, (2) one or more helper constructs comprising the viral functions missing from the AAV vector, (3) the gene of interest (GOI), and (4) a plasmid construct containing the genome of the AAV vector, which comprises the engineered guide RNA sequences.

Constructs were designed as shown in Table 8.

TABLE 8
gRNA AAV Constructs.

| | gRNA SEQ | |
|---|---|---|
| Name | ID NO: | #gRNA copies |
| gRNA1 + reg1 | SEQ ID NO: | 2 |
| | 375 | |
| gRNA2 + reg1 | SEQ ID NO: | 2 |
| | 376 | |
| gRNA1 + reg2 | SEQ ID NO: | 2 |
| | 375 | |
| gRNA1 + reg3 | SEQ ID NO: | 2 |
| | 375 | |
| gRNA2 + reg3 | SEQ ID NO: | 2 |
| | 376 | |
| gRNA1 + reg4 | SEQ ID NO: | 2 |
| | 375 | |
| Control gRNA | — | 1 |
| (RAB7A) (in- | | |
| vitro) | | |
| Control gRNA | — | 2 |
| (LCOR) (in- | | |
| vivo) | | |

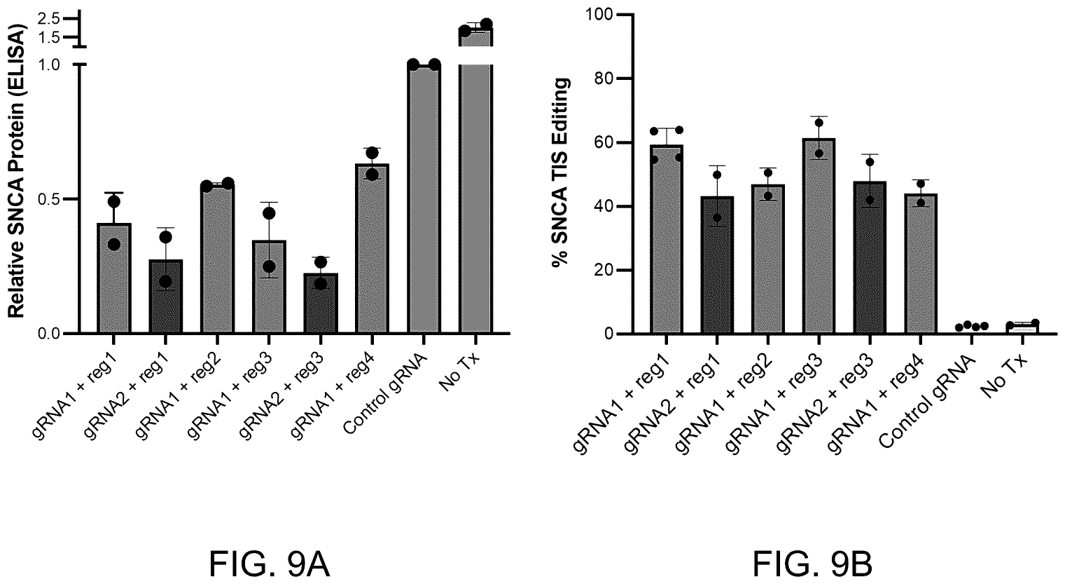

AAV-gRNA vectors were delivered to the primary neurons at 1×10⁵ vector genomes (vg) per cell (vg/cell) on after 5 days in cell culture and harvested for all measurements at passage 12. SNCA protein was measured via ELISA. SNCA-TIS targeting gRNA-treated primary mouse neurons exhibited 40 to 80% of protein knockdown relative to control gRNA (FIG. 9A). RNA editing was measured via Sanger Sequencing. SNCA-TIS targeting gRNA-treated primary mouse neurons exhibited 40 to 60% RNA editing (FIG. 9B). Strong wildtype alpha-synuclein protein knockdown was shown with gRNA2 constructs having the SEQ ID NO: 293 footprint.

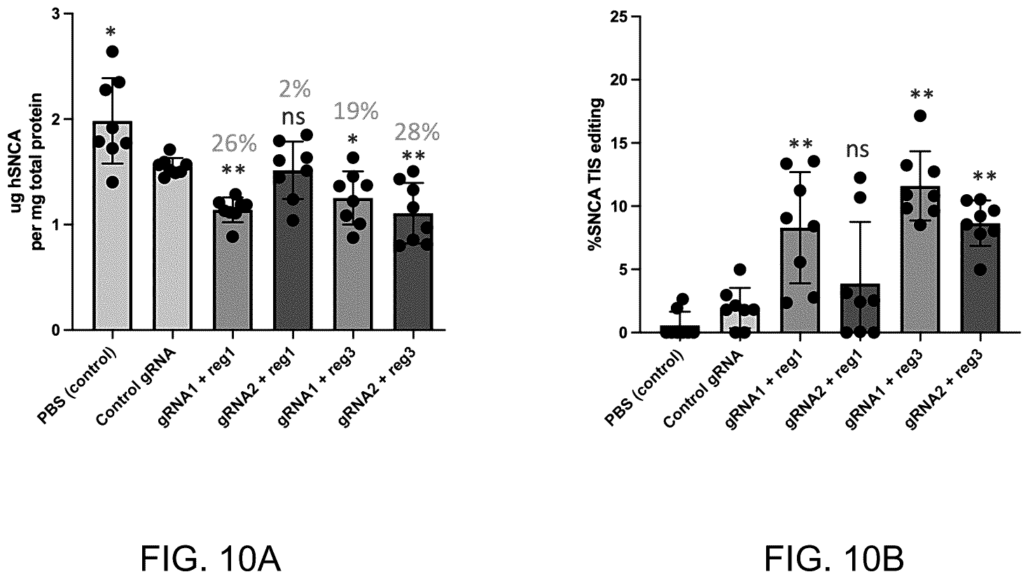

Next, transgenic mice were injected with the AAV-gRNA vectors by bilateral intracerebroventricular injection (ICV) at 1.55×10¹¹ viral genomes per mouse. RNA editing and alpha-synuclein protein was measured 4 weeks post-injection. SNCA protein was measured via ELISA and SNCA editing was measured via Sanger sequencing. Significant alpha-synuclein protein knockdown was observed in three of four treatment groups. Mice treated with the AAV-gRNA vectors encoding the SNCA-targeting guide RNAs exhibited up to 48% knockdown of SNCA protein in mouse brain relative to controls and 28% knockdown of SNCA on average (FIG. 10A). Approximately 5 to 18% RNA editing was observed in the mouse brain (FIG. 10B). Functional knockdown of alpha-synuclein protein in mouse CNS tissues was observed for gRNA1 (SEQ ID NO: 375) and gRNA2 (SEQ ID NO: 376) 4 weeks post-bilateral ICV.

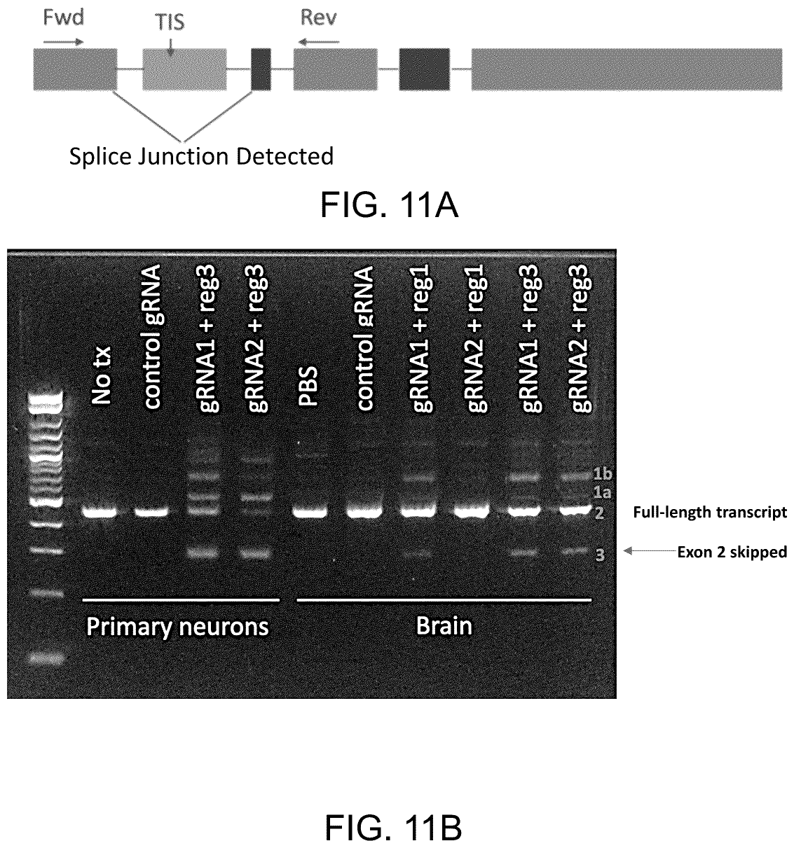

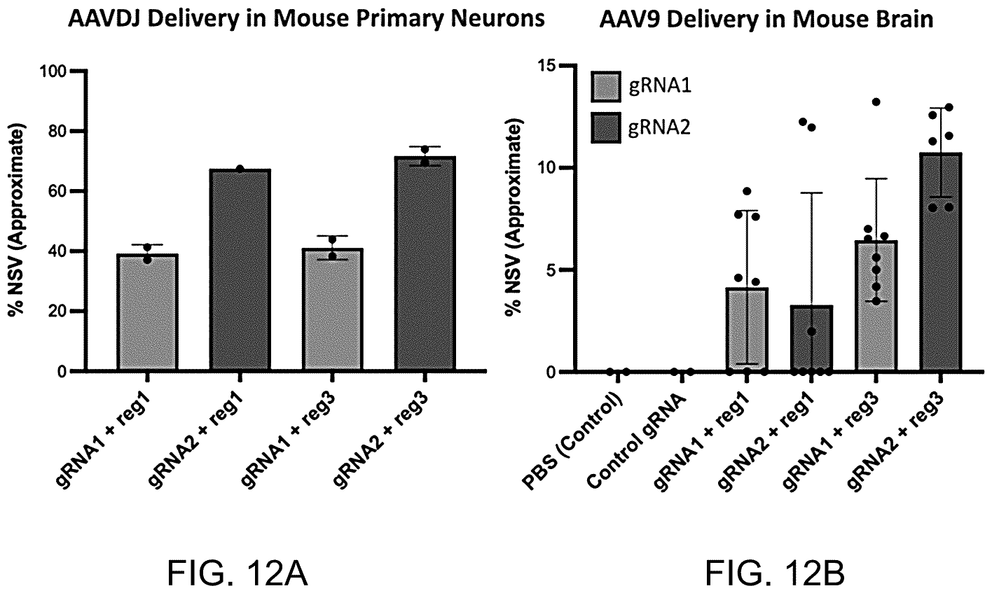

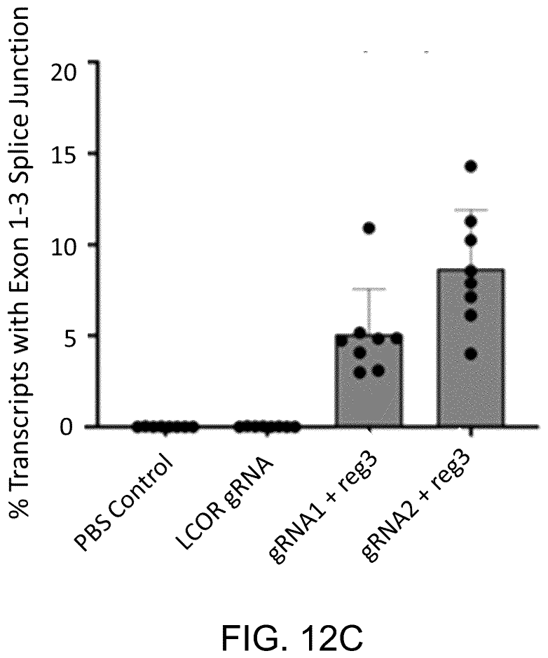

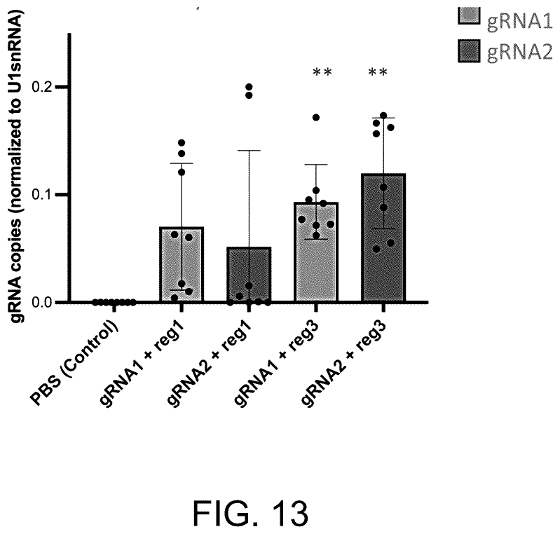

RT-PCR amplification of SNCA mRNA transcripts between Exons 1-4 of SNCA was performed to characterize any alternative splicing present in the mRNA transcripts resulting from gRNA targeting of the SNCA TIS. This analysis demonstrated the presence of gRNA-mediated skipping of the TIS containing exon 2 of SNCA, with transcript in both mouse primary neurons and whole brain showing exclusion of SNCA Exon 2 due to the ADAR-mediated editing (FIGS. 11A-11B) which results in the observed reduction in wildtype alpha-synuclein protein expression. Densitometry was performed to approximate the percent of the non-canonical alpha-synuclein splice variants detected in primary neurons and mice brain due to the ADAR-mediated editing of the Codon 2 TIS by the guide RNAs (FIGS. 12A-12C). The rate of splicing was dependent on gRNA architecture as gRNA2 exhibited increased novel splice variant formation (>60%) in mouse primary neurons (FIG. 12A). Droplet digital polymerase chain reaction (ddPCR) was performed to determine the percentage of transcript with the exon 1-3 splice junction. The exon 1-3 splice junction was detected in 5-10% of total SNCA transcripts in-vivo (FIG. 12C). Viral genome and guide RNA quantification were performed to evaluate editing readouts. The gRNA copies and viral DNA amount were measured via PCR. Viral genomes were detected per diploid genome (0-12 vg/dg) as shown in FIG. 13.

### Example 6

### SNCA-Targeting gRNA Validation in Human Neural Stem Cell (NSC)-Derived and Induced Pluripotent Stem Cell (iPSC)-Derived Neurons

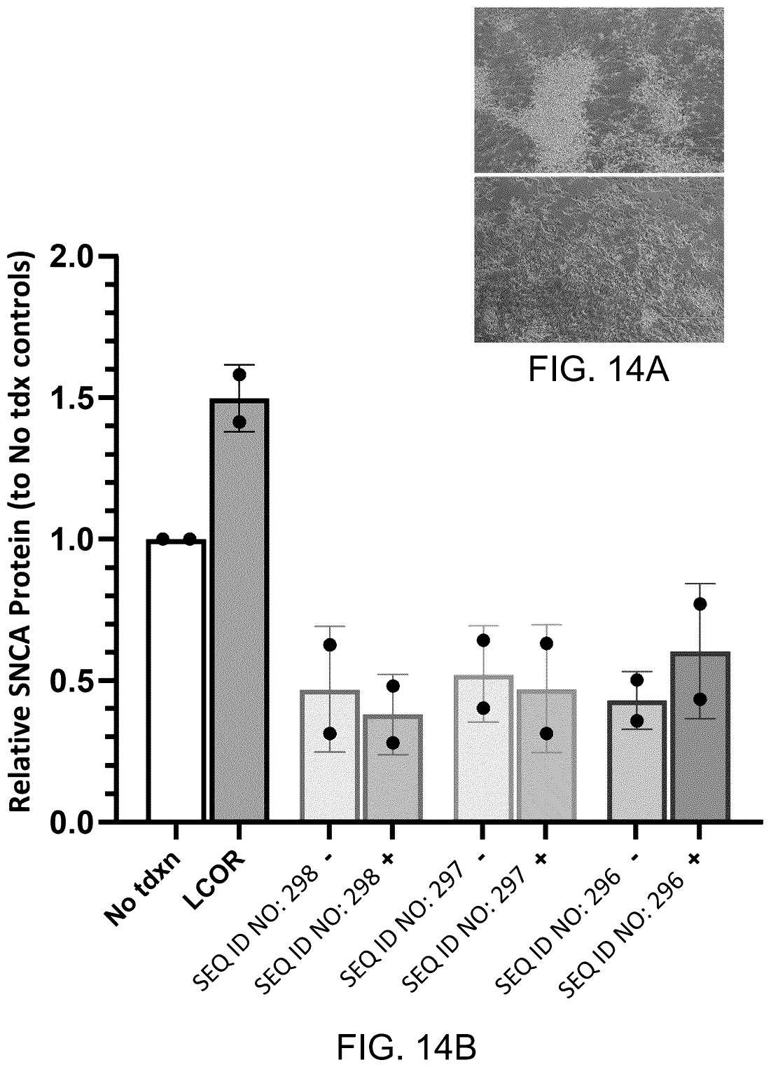

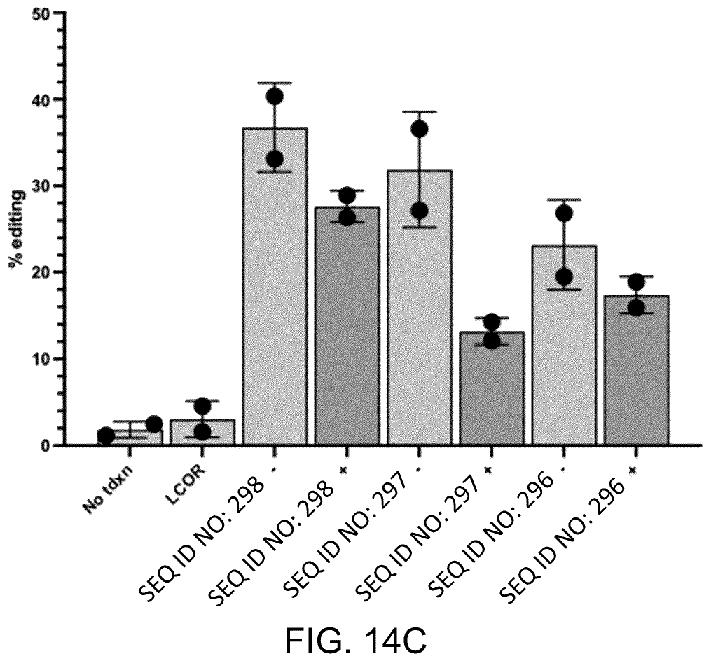

Human neural stem cell (NSC)-derived and induced pluripotent stem cells (iPSC)-derived dopaminergic neurons were cultured according to standard protocols. Substantial variability in cell population (% neurons vs. other cell types) was observed with the NSC-derived neurons and was dependent on differentiation (FIG. 14A). The following constructs in Table 9, were utilized in this example:

TABLE 9
gRNA AAV Constructs.

| | | Trans- |
|---|---|---|
| Base Guide RNA | hnRNP | duction |
| Footprint | A1 | Marker |
| CAGCAGCCACAACUCCCUCG | Double | CMV350- |
| CGCAACUUUGAAAGUCCUUU | | Thy1.2 |
| CAUGCAUAGAUCCACGGCUA | | |
| CAUAAUUCCUUUUGGAGUCA | | |
| CUGUCGUCGA (SEQ ID | | |
| NO: 297) | | |
| CAGCAGCCACAACUCCCUCG | None | CMV350- |
| CGCAACUUUGAAAGUCCUUU | | Thy1.2 |
| CAUGCAUAGAUCCACGGCUA | | |
| CAUAAUUCCUUUUGGAGUCA | | |
| CUGUCGUCGA (SEQ ID | | |
| NO: 297) | | |
| CUCAGCAGCAGCCACAACUC | Double | CMV350- |
| CAAUGAAGGCCUUUGAAAGU | | Thy1.2 |
| CCUUUCAUGAAUACAUCUAC | | |
| GGCUACAUAUCUCCUUUACA | | |
| CCACACUGUCGUCGAAUGGC | | |
| (SEQ ID NO: 298) | | |
| CUCAGCAGCAGCCACAACUC | None | CMV350- |
| CAAUGAAGGCCUUUGAAAGU | | Thy1.2 |
| CCUUUCAUGAAUACAUCUAC | | |
| GGCUACAUAUCUCCUUUACA | | |
| CCACACUGUCGUCGAAUGGC | | |
| (SEQ ID NO: 298) | | |
| GCCACAACUCCCUCCUUGAU | Double | CMV350- |
| AGGCGAAAGUCCUUUCAUGA | | Thy1.2 |
| AUAACUCCACGGCUAUCAUA | | |
| CUCCUUUACACCACACUGUC | | |
| GUCGA (SEQ ID NO: | | |
| 296) | | |
| GCCACAACUCCCUCCUUGAU | None | CMV350- |
| AGGCGAAAGUCCUUUCAUGA | | Thy1.2 |
| AUAACUCCACGGCUAUCAUA | | |
| CUCCUUUACACCACACUGUC | | |
| GUCGA (SEQ ID NO: | | |
| 296) | | |

Human NSC-derived neurons or iPSC neurons were infected with AAV-SNCA gRNAs at 5×10⁴ vg/cell for 7 days (NSC-derived) or 5 days (iPSC-derived). Despite differences in the cell culture, transduction efficiency of the AAV-gRNAs ranged from about 30% to 40% Thy1.2 positive cells. For the NSC-derived neurons, a higher transduction efficiency was associated with decreased SNCA protein (FIG. 14B) and increased levels of editing of the SNCA TIS (FIG. 14C). As shown in FIG. 14B, the AAV vectors produced a 40-60% reduction in wild-type alpha-synuclein protein. As shown in the graph provided in FIG. 14C, AAV packaged gRNAs delivered to the NSC-derived neurons facilitated 20-40% RNA editing of SNCA.

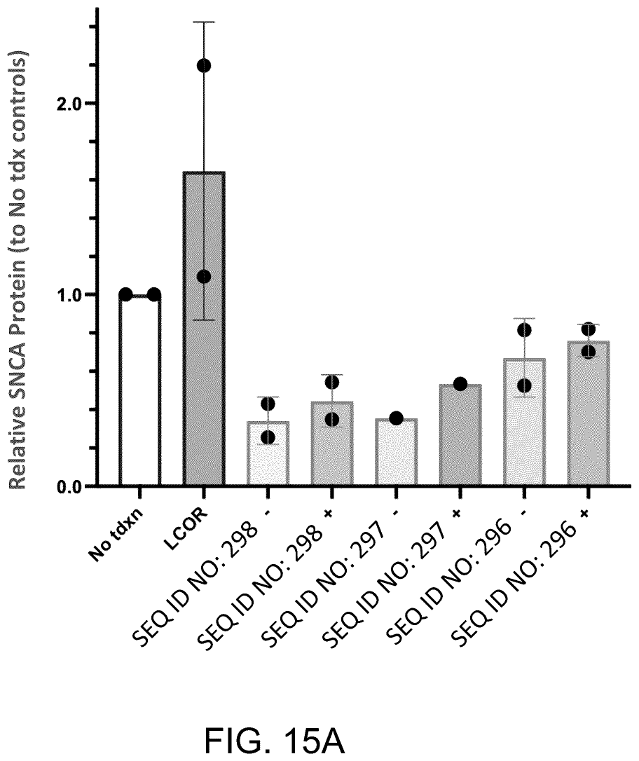

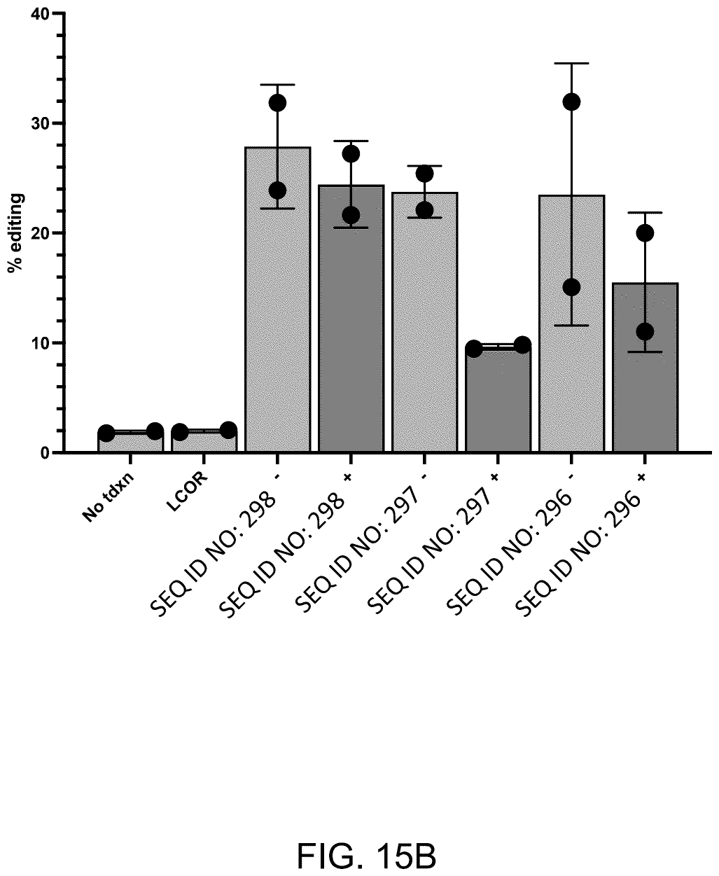

As shown in FIG. 15A, the AAV vectors produced a 30-70% reduction in wild-type alpha-synuclein protein in the iPSC-derived neurons, and a similar level of edited SNCA TIS transcripts (FIG. 15B) as seen in the NSC-derived neurons (FIG. 14C).

Further, an SNCA novel splice variant (NSV) assay was performed with each cell line. The amount of transcript with a novel splice junction between human SNCA exons 1 and 3 were measured. The percentage of exon 2 skipped was determined by the following formula

$$\% Exon 2 skipped = ( \frac{“ Skip ” probe}{“ WT ” + “ Skip ” probes} ) * 100 .$$

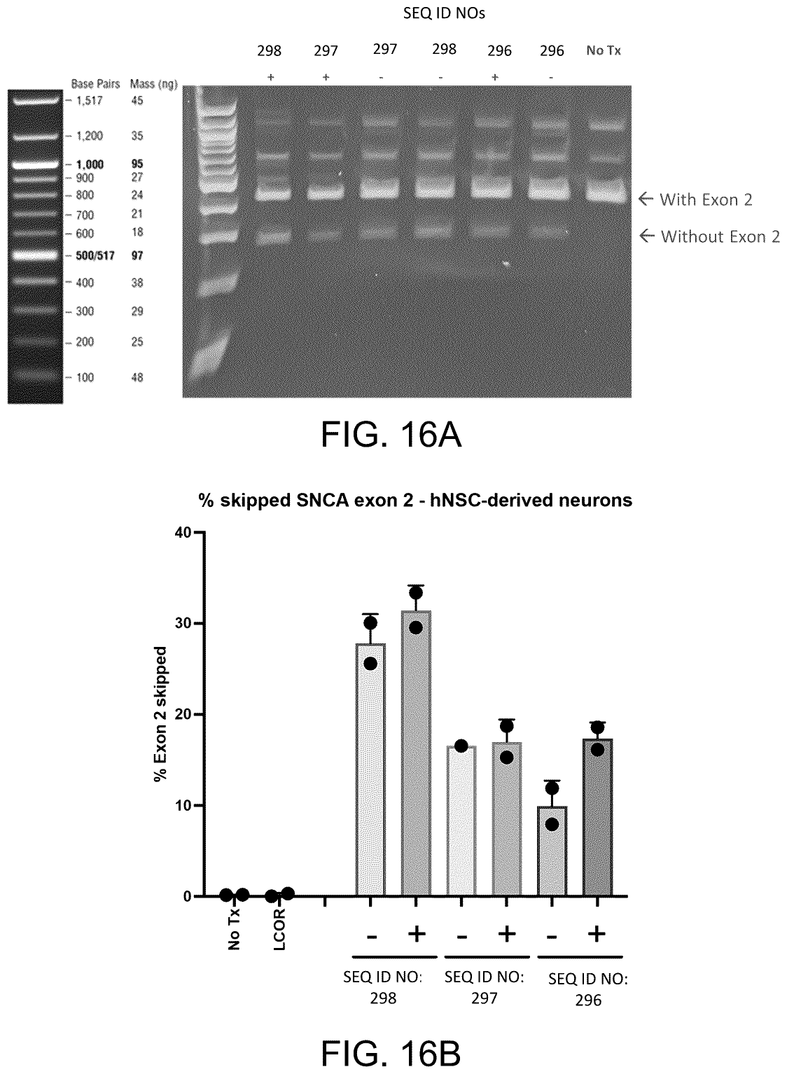

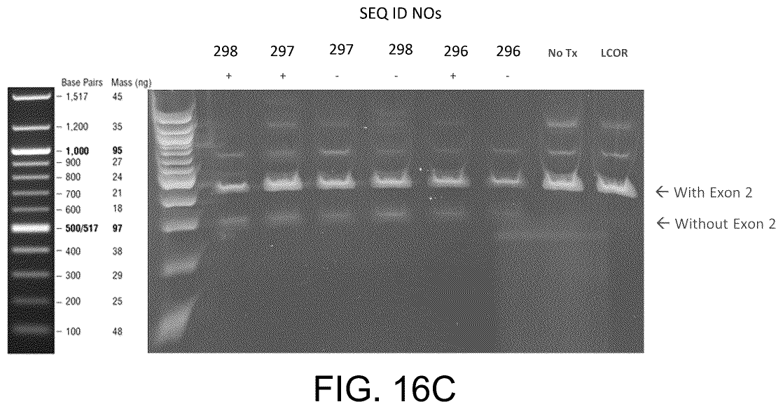

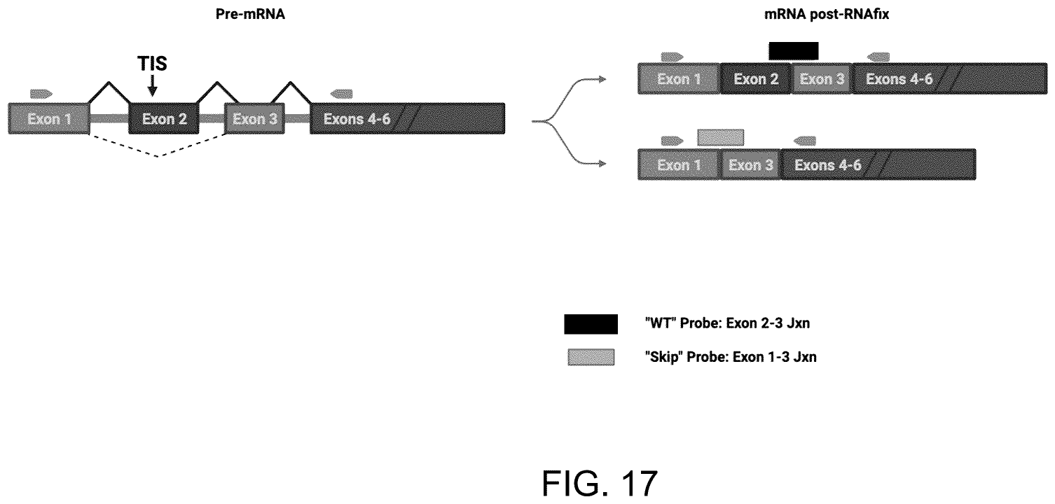

The AAV-SNCA gRNAs induced significant SNCA exon 2 skipping relative to controls in both the NSC-derived neurons (FIGS. 16A-16B) and the iPSC-derived neurons (FIG. 16C). A schematic of SNCA pre-mRNA and the proposed mRNA splicing products (both full length and the Exon 2 splice variant) post-gRNA delivery is shown in FIG. 17.

### Example 7

### Vector-Packaged SNCA-TIS Targeting gRNAs

Engineered guide RNAs targeting the SNCA-TIS gRNAs of SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 334, and SEQ ID NO: 340 are packaged into AAV vectors for AAV-mediated delivery of guide RNAs into cells. Each AAV vector cassette includes one or two copies of an engineered guide RNA targeting the SNCA-TIS. The AAV-gRNA constructs encoding the guide RNAs that target the SNCA TIS are used to deliver the engineered guide RNA into cells.

### Example 8

### Barbell Modifications of SNCA Guide RNAs and Non-Coding −4 Reducing Guide RNAs

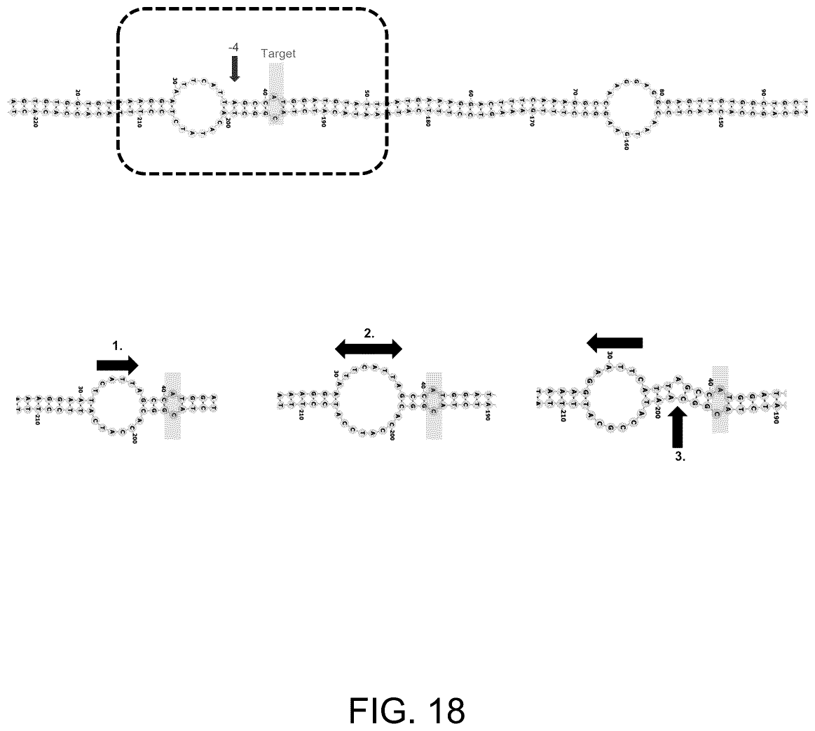

SNCA parental guide RNAs SEQ ID NO: 293 and SEQ ID NO: 296 were further engineered to reduce off-target effects by modifying the left barbell structure. The barbell was moved closer to the targeting site, increased in size, and/or U-deletions were introduced at the site of the −4 position (FIG. 18). Shifting the left barbell diminished −4 editing while retaining efficiency. An optimal reduction of −4 editing and retention of on-target efficiency was observed for (−4, 33) 6/6 design and (−4, 33) 8/8 designs relative to the parental SEQ ID NO: 293 RNA guide. Editing at the −4 site was reduced with a minor impact to efficiency with a (−8,33) 6/6 UDel relative to the parental guide.

Additional mutations were performed on the constructs to further deviate from the parent sequence. Specifically, additional wobble base pairs were added barbell-modified guide RNAs. Sequences of the guides are provided in Table 2 and summarized in Table 10 below with corresponding structural features.

TABLE 10
SNCA-targeting gRNAs.

| RNA | | DNA | | |
|---|---|---|---|---|
| SEQ | | SEQ | | |
| ID | | ID | | |
| NO: | RNA Sequence | NO: | DNA Sequence | Structural Features |
| 333 | CAGCAGCCACAACUC | 325 | CAGCAGCCACAACT | -6_6-6_internal_loop-symmetric_ |
| | CCGAAGGAGCCUUUG | | CCCGAAGGAGCCTT | AUUCAU-UACUUA |
| | AAAGUCCUUAAUUGA | | TGAAAGTCCTTAAT | 0_1-1_mismatch_A-C |
| | AUACAUCCACGGCUA | | TGAATACATCCACG | 13_3-3_bulge-symmetric_UGA-AAU |
| | UACUUAUCCUUUACA | | GCTATACTTATCCT | 31_0-1_bulge-asymmetric_-A |
| | CCACACUGUCGUCGA | | TTACACCACACTGT | 33 5-4_internal_loop-asymmetric_ |
| | AUGGC | | CGTCGAATGGC | AAGGA-GAAG |
| | | | | 55_1-1_mismatch_C-A |
| 390 | CAGCAGCCACAACUC | 724 | CAGCAGCCACAACT | -40_1-1_wobble_G-U |
| | CCGAAGGAGCCUUUG | | CCCGAAGGAGCCTT | -6_6-6_internal_loop-symmetric_ |
| | AAAGUCCUUAAUUGA | | TGAAAGTCCTTAAT | AUUCAU-UACUUA |
| | AUACAUCCACGGCUA | | TGAATACATCCACG | 0_1-1_mismatch A-C |
| | UACUUAUCCUUUACA | | GCTATACTTATCCT | 13_3-3_bulge-symmetric_UGA-AAU |
| | CCACACUGUCGUCGA | | TTACACCACACTGT | 31_0-1_bulge-asymmetric_-A |
| | AUGGU | | CGTCGAATGGT | 33_5-4_internal_loop-asymmetric_ |
| | | | | AAGGA-GAAG |
| | | | | 55_1-1_mismatch_C-A |
| 334 | CAGCAGCCACAACUC | 325 | CAGCAGCCACAACT | -40_1-1_wobble_G-U |
| | CCGAAGGAGCCUUUG | | CCCGAAGGAGCCTT | -36_1-1_wobble_U-G |
| | AAAGUCCUUAAUUGA | | TGAAAGTCCTTAAT | -33_1-1_wobble_G-U |
| | AUACAUCCACGGCUA | | TGAATACATCCACG | -6_6-6_internal_loop-symmetric_ |
| | UACUUAUCCUUUACA | | GCTATACTTATCCT | AUUCAU-UACUUA |
| | CCACACUGUCGUUGA | | TTACACCACACTGT | 0_1-1_mismatch A-C |
| | GUGGU | | CGTCGAATGGC | 13_3-3_bulge-symmetric_UGA-AAU |
| | | | | 31_0-1_bulge-asymmetric_-A |
| | | | | 33_5-4_internal_loop-asymmetric_ |
| | | | | AAGGA-GAAG |
| | | | | 55_1-1_mismatch_C-A |
| 335 | CAGCAGCCACAACUC | 326 | CAGCAGCCACAACT | -40_1-1_wobble_G-U |
| | CCGAAGGAGCCUUUG | | CCCGAAGGAGCCTT | -36_1-1_wobble_U-G |
| | AAAGUCCUUAAUUGA | | TGAAAGTCCTTAAT | -33_1-1_wobble_G-U |
| | AUACAUCCACGGCUA | | TGAATACATCCACG | -30_1-1_wobble_G-U |
| | UACUUAUCCUUUACA | | GCTATACTTATCCT | -25_1-1_wobble_U-G |
| | CCACGCUGUUGUUGA | | TTACACCACACTGT | -6_6-6_internal_loop-symmetric_ |
| | GUGGU | | CGTTGAGTGGT | AUUCAU-UACUUA |
| | | | | 0_1-1_mismatch A-C |
| | | | | 13_3-3_bulge-symmetric_UGA-AAU |
| | | | | 31_0-1_bulge-asymmetric_-A |
| | | | | 33_5-4_internal_loop-asymmetric_ |
| | | | | AAGGA-GAAG |
| | | | | 55_1-1_mismatch_C-A |
| 391 | CAGCAGCCACAACUC | 725 | CAGCAGCCACAACT | -40_1-1_wobble_G-U |
| | CCGAAGGAGCCUUUG | | CCCGAAGGAGCCTT | -36_1-1_wobble_U-G |
| | AAAGUCCUUAAUUGA | | TGAAAGTCCTTAAT | -33_1-1_wobble_G-U |
| | AUACAUCCACGGCUA | | TGAATACATCCACG | -30_1-1_wobble_G-U |
| | UACUUAUCCUUUGCA | | GCTATACTTATCCT | -25_wobble_U-G |
| | UCACGCUGUUGUUGA | | TTGCATCACGCTGT | -21_1-1_wobble_G-U |
| | GUGGU | | TGTTGAGTGGT | -18_1-1_wobble_U-G |
| | | | | -6_6-6_internal_loop-symmetric_ |
| | | | | AUUCAU-UACUUA |
| | | | | 0_1-1_mismatch_A-C |
| | | | | 13_3-3_bulge-symmetric_UGA-AAU |
| | | | | 31_0-1_bulge-asymmetric_-A |
| | | | | 33_5-4_internal_loop-asymmetric_ |
| | | | | AAGGA-GAAG |
| | | | | 55_1-1_mismatch_C-A |
| 336 | CAGCAGCCACAACUC | 328 | CAGCAGCCACAACT | -40_1-1_wobble_G-U |
| | CCGAAGGAGCCUUUG | | CCCGAAGGAGCCTT | -36_wobble_U-G |
| | AAAGUCCUUAAUUGA | | TGAAAGTCCTTAAT | -33_1-1_wobble_G-U |
| | AUACAUCCACGGUUA | | TGAATACATCCACG | -30_1-1_wobble_G-U |
| | UACUUAUUCUUUGCA | | GTTATACTTATTCT | -25_1-1_wobble_U-G |
| | UCACGCUGUUGUUGA | | TTGCATCACGCTGT | -21_1-1_wobble_G-U |
| | GUGGU | | TGTTGAGTGGT | -18_1-1_wobble_U-G |
| | | | | -13_wobble_G-U |
| | | | | -6_6-6_internal_loop-symmetric_ |
| | | | | AUUCAU-UACUUA |
| | | | | -3_1-1_wobble_G-U |
| | | | | 0_1-1_mismatch A-C |
| | | | | 13_3-3_bulge-symmetric_UGA-AAU |
| | | | | 31_0-1_bulge-asymmetric_-A |
| | | | | 33 5-4_internal_loop-asymmetric_ |
| | | | | AAGGA-GAAG |
| | | | | 55_1-1_mismatch_C-A |
| 392 | CAGCAGCCACAACUC | 726 | CAGCAGCCACAACT | -40_1-1_wobble_G-U |
| | CCGAAGGAGCCUUUG | | CCCGAAGGAGCCTT | -36_1-1_wobble_U-G |
| | AAAGUCCUUAAUUGA | | TGAAAGTCCTTAAT | -33_1-1_wobble_G-U |
| | GUAUAUCCACGGUUA | | TGAGTATATCCACG | -30_1-1_wobble_G-U |
| | UACUUAUUCUUUGCA | | GTTATACTTATTCT | -25_wobble_U-G |
| | UCACGCUGUUGUUGA | | TTGCATCACGCTGT | -21_wobble_G-U |
| | GUGGU | | TGTTGAGTGGT | -18_1-1_wobble_U-G |
| | | | | -13_1-1_wobble_G-U |
| | | | | -6_6-6_internal_loop-symmetric_ |
| | | | | AUUCAU-UACUUA |
| | | | | -3_1-1_wobble_G-U |
| | | | | 0_1-1_mismatch A-C |
| | | | | 6_1-1_wobble_G-U |
| | | | | 9_1-1_wobble_U-G |
| | | | | 13_3-3_bulge-symmetric_UGA-AAU |
| | | | | 31_0-1_bulge-asymmetric_-A |
| | | | | 33 5-4_internal_loop-asymmetric_ |
| | | | | AAGGA-GAAG |
| | | | | 55_1-1_mismatch_C-A |
| 393 | CAGCAGCCACAACUC | 727 | CAGCAGCCACAACT | -40_1-1_wobble_G-U |
| | UCGAAGGAGCCUUUG | | CTCGAAGGAGCCTT | -36_1-1_wobble_U-G |
| | AGAGUCCUUAAUUGA | | TGAGAGTCCTTAAT | -33_1-1_wobble_G-U |
| | GUAUAUCCACGGUUA | | TGAGTATATCCACG | -30_1-1_wobble_G-U |
| | UACUUAUUCUUUGCA | | GTTATACTTATTCT | -25_1-1_wobble_U-G |
| | UCACGCUGUUGUUGA | | TTGCATCACGCTGT | -21_1-1_wobble_G-U |
| | GUGGU | | TGTTGAGTGGT | -18_1-1_wobble_U-G |
| | | | | -13_1-1_wobble_G-U |
| | | | | -6_6-6_internal_loop-symmetric_ |
| | | | | AUUCAU-UACUUA |
| | | | | -3_1-1_wobble_G-U |
| | | | | 0_1-1_mismatch A-C |
| | | | | 6_1-1_wobble_G-U |
| | | | | 9_1-1_wobble_U-G |
| | | | | 13_3-3_bulge-symmetric_UGA-AAU |
| | | | | 23_1-1_wobble_U-G |
| | | | | 31_0-1_bulge-asymmetric_-A |
| | | | | 33 5-4_internal_loop-asymmetric_ |
| | | | | AAGGA-GAAG |
| | | | | 40_1-1_wobble_G-U |
| | | | | 55_1-1_mismatch_C-A |
| 394 | CAGUAGCCACGACUC | 728 | CAGTAGCCACGACT | -40_1-1_wobble_G-U |
| | UCGAAGGAGCCUUUG | | CTCGAAGGAGCCTT | -36_1-1_wobble_U-G |
| | AGAGUCCUUAAUUGA | | TGAGAGTCCTTAAT | -33_1-1_wobble_G-U |
| | GUAUAUCCACGGUUA | | TGAGTATATCCACG | -30_1-1_wobble_G-U |
| | UACUUAUUCUUUGCA | | GTTATACTTATTCT | -25_1-1_wobble_U-G |
| | UCACGCUGUUGUUGA | | TTGCATCACGCTGT | -21_1-1_wobble_G-U |
| | GUGGU | | TGTTGAGTGGT | -18_1-1_wobble_U-G |
| | | | | -13_1-1_wobble_G-U |
| | | | | -6_6-6_internal_loop-symmetric_ |
| | | | | AUUCAU-UACUUA |
| | | | | -3_1-1_wobble_G-U |
| | | | | 0_1-1_mismatch A-C |
| | | | | 6_1-1_wobble_G-U |
| | | | | 9_1-1_wobble_U-G |
| | | | | 13_3-3_bulge-symmetric_UGA-AAU |
| | | | | 23_1-1_wobble_U-G |
| | | | | 31_0-1_bulge-asymmetric_-A |
| | | | | 33_5-4_internal_loop-asymmetric_ |
| | | | | AAGGA-GAAG |
| | | | | 40_1-1_wobble_G-U |
| | | | | 45_1-1_wobble_U-G |
| | | | | 52_1-1_wobble_G-U |
| | | | | 55_1-1_mismatch_C-A |
| 395 | CAGCAGCCACAACUC | 729 | CAGCAGCCACAACT | |
| | CCGAAGGAGCCUUUG | | CCCGAAGGAGCCTT | |
| | AAAGUCCUUUAUUGA | | TGAAAGTCCTTTAT | |
| | AUACAUCCACGGCUA | | TGAATACATCCACG | |
| | AUGAAAUCCUUUACA | | GCTAATGAAATCCT | |
| | CCACACUGUCGUCGA | | TTACACCACACTGT | |
| | AUGGC | | CGTCGAATGGC | |
| 337 | CUCAGCAGCAGCCAC | 329 | CTCAGCAGCAGCCA | -40_1-1_wobble_G-U |
| | AACUCCGUGCGGGGC | | CAACTCCGTGCGGG | -36_1-1_wobble_U-G |
| | CUUUGAAAGUCCUUU | | GCCTTTGAAAGTCC | -30_1-1_wobble_G-U |
| | CAUGAAUACAUCUAC | | TTTCATGAATACAT | -6_6-6_internal_loop-symmetric_ |
| | GGCUAUACUGGUCCU | | CTACGGCTATACTG | AUUCAU-UACUGG |
| | UUACACCACACUGUU | | GTCCTTTACACCAC | 0_1-1_mismatch_A-C |
| | GUCGAGUGGU | | ACTGTTGTCGAGTG | 2_1-1_wobble_G-U |
| | | | GT | 33_2-2_bulge-symmetric_AA-GG |
| | | | | 36_1-1_mismatch_G-G |
| | | | | 38_1-1_mismatch_G-G |
| 338 | CUCAGCAGCAGCCAC | 330 | CTCAGCAGCAGCCA | -40_1-1_wobble_G-U |
| | AACUCCGUGCGGGGC | | CAACTCCGTGCGGG | -36_1-1_wobble_U-G |
| | CUUUGAAAGUCCUUU | | GCCTTTGAAAGTCC | -30_1-1_wobble_G-U |
| | CAUGAAUACAUCUAC | | TTTCATGAATACAT | -25_1-1_wobble_U-G |
| | GGCUAUACUGGUCCU | | CTACGGCTATACTG | -23_1-1_wobble_U-G |
| | UUACACCGCGCUGUU | | GTCCTTTACACCGC | -6_6-6_internal_loop-symmetric_ |
| | GUCGAGUGGU | | GCTGTTGTCGAGTG | AUUCAU-UACUGG |
| | | | GT | 0_1-1_mismatch_A-C |
| | | | | 2_1-1_wobble_G-U |
| | | | | 33_2-2_bulge-symmetric AA-GG |
| | | | | 36_1-1_mismatch_G-G |
| | | | | 38_1-1_mismatch_G-G |
| 339 | CUCAGCAGCAGCCAC | 331 | CTCAGCAGCAGCCA | -40_1-1_wobble_G-U |
| | AACUCCGUGCGGGGC | | CAACTCCGTGCGGG | -36_1-1_wobble_U-G |
| | CUUUGAAAGUCCUUU | | GCCTTTGAAAGTCC | -30_1-1_wobble_G-U |
| | CAUGAAUACAUCUAC | | TTTCATGAATACAT | -25_1-1_wobble_U-G |
| | GGCUAUACUGGUCCU | | CTACGGCTATACTG | -23_1-1_wobble_U-G |
| | UUGCAUCGCGCUGUU | | GTCCTTTGCATCGC | -21_1-1_wobble_G-U |
| | GUCGAGUGGU | | GCTGTTGTCGAGTG | -18_1-1_wobble_U-G |
| | | | GT | -6_6-6_internal_loop-symmetric_ |
| | | | | AUUCAU-UACUGG |
| | | | | 0_1-1_mismatch A-C |
| | | | | 2_1-1_wobble_G-U |
| | | | | 33_2-2_bulge-symmetric_AA-GG |
| | | | | 36_1-1_mismatch_G-G |
| | | | | 38_1-1_mismatch_G-G |
| 340 | CUCAGCAGCAGCCAC | 332 | CTCAGCAGCAGCCA | -40_1-1_wobble_G-U |
| | AACUCCGUGCGGGGC | | CAACTCCGTGCGGG | -36_1-1_wobble_U-G |
| | CUUUGAAAGUCCUUU | | GCCTTTGAAAGTCC | -30_1-1_wobble_G-U |
| | CAUGAAUACGUCUAC | | TTTCATGAATACGT | -25_1-1_wobble_U-G |
| | GGCUAUACUGGUUCU | | CTACGGCTATACTG | -23_1-1_wobble_U-G |
| | UUGCAUCGCGCUGUU | | GTTCTTTGCATCGC | -21_1-1_wobble_G-U |
| | GUCGAGUGGU | | GCTGTTGTCGAGTG | -18_wobble_U-G |
| | | | GT | -13_1-1_wobble_G-U |
| | | | | -6_6-6_internal_loop-symmetric_ |
| | | | | AUUCAU-UACUGG |
| | | | | 0_1-1_mismatch_A-C |
| | | | | 2_1-1_wobble_G-U |
| | | | | 5_1-1_wobble_U-G |
| | | | | 33_2-2_bulge-symmetric_AA-GG |
| | | | | 36_1-1_mismatch_G-G |
| | | | | 38_1-1_mismatch_G-G |
| 396 | CUCAGCAGCAGCCAC | 730 | CTCAGCAGCAGCCA | -4_8-8_internal_loop- |
| | AACUCCGUGGUAGGC | | CAACTCCGTGGTAG | symmetric_AUUCAUUA-AUUCCUUC |
| | CUUUGAAAGUCCUUU | | GCCTTTGAAAGTCC | 0_1-1_mismatch A-C |
| | CAUGAAUACAUCUAC | | TTTCATGAATACAT | 2_1-1_wobble_G-U |
| | GGCAUUCCUUCUCCU | | CTACGGCATTCCTT | 33_4-4_bulge-symmetric_AAGG-GGUA |
| | UUACACCACACUGUC | | CTCCTTTACACCAC | 38_1-1_mismatch_G-G |
| | GUCGAAUGGC | | ACTGTCGTCGAATG | |
| | | | GC | |
| 397 | CUCAGCAGCGGCCAC | 731 | CTCAGCAGCGGCCA | -36_wobble_U-G |
| | AGCUCCAUGGAAGGC | | CAGCTCCATGGAAG | -33_wobble_G-U |
| | UUUUGGAAGUUCUUU | | GCTTTTGGAAGTTC | -21_1-1_wobble_G-U |
| | UAUGAGUACGUCUAC | | TTTTATGAGTACGT | -18_1-1_wobble_U-G |
| | GGCAUCACUCCUCCU | | CTACGGCATCACTC | -4_8-8_internal_loop- |
| | UUGCAUCACACUGUC | | CTCCTTTGCATCAC | symmetric_AUUCAUUA-AUCACUCC |
| | GUUGAGUGGC | | ACTGTCGTTGAGTG | 0_1-1_mismatch_A-C |
| | | | GC | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_6-6_internal_loop-symmetric_ |
| | | | | AAGGAG-AUGGAA |
| | | | | 43_wobble_U-G |
| | | | | 50_1-1_wobble_U-G |
| 398 | CUCAGCAGCGGCCAC | 732 | CTCAGCAGCGGCCA | -36_1-1_wobble_U-G |
| | AGCUCCGUGGAAGGC | | CAGCTCCGTGGAAG | -33_1-1_wobble_G-U |
| | UUUUGGAAGUUCUUU | | GCTTTTGGAAGTTC | -21_1-1_wobble_G-U |
| | UAUGAGUACGUCUAC | | TTTTATGAGTACGT | -18_wobble_U-G |
| | GGCAUCCCUCCUCCU | | CTACGGCATCCCTC | -4_8-8_internal_loop- |
| | UUGCAUCACACUGUC | | CTCCTTTGCATCAC | symmetric_AUUCAUUA-AUCCCUCC |
| | GUUGAGUGGC | | ACTGTCGTTGAGTG | 0_1-1_mismatch_A-C |
| | | | GC | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric_AAGG-GGAA |
| | | | | 38_1-1_mismatch_G-G |
| | | | | 43_1-1_wobble_U-G |
| | | | | 50_1-1_wobble_U-G |
| 399 | CUCAGUAGCGGCCAC | 733 | CTCAGTAGCGGCCA | -36_1-1_wobble_U-G |
| | AGCUCCGUGGAAGGC | | CAGCTCCGTGGAAG | -33_1-1_wobble_G-U |
| | UUUUGGAAGUUCUUU | | GCTTTTGGAAGTTC | -21_1-1_wobble_G-U |
| | UAUGAGUACGUCUAC | | TTTTATGAGTACGT | -18_wobble_U-G |
| | GGCAUCCCUCCUCCU | | CTACGGCATCCCTC | -4_8-8_internal_loop- |
| | UUGCAUCACACUGUC | | CTCCTTTGCATCAC | symmetric_AUUCAUUA-AUCCCUCC |
| | GUUGAGUGGC | | ACTGTCGTTGAGTG | 0_1-1_mismatch A-C |
| | | | GC | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric AAGG-GGAA |
| | | | | 38_mismatch_G-G |
| | | | | 43_wobble_U-G |
| | | | | 50_1-1_wobble_U-G |
| | | | | 54_1-1_wobble_G-U |
| 400 | CUCAGUAGCGGCCAC | 734 | CTCAGTAGCGGCCA | -36_1-1_wobble_U-G |
| | AGCUCCGUGGAAGGC | | CAGCTCCGTGGAAG | -33_1-1_wobble_G-U |
| | UUUUGGAAGUUCUUU | | GCTTTTGGAAGTTC | -21_1-1_wobble_G-U |
| | UAUGAGUACGUCUAC | | TTTTATGAGTACGT | -18_1-1_wobble_U-G |
| | GGCAUUCCUCCUCCU | | CTACGGCATTCCTC | -4_8-8_internal_loop- |
| | UUGCAUCACACUGUC | | CTCCTTTGCATCAC | symmetric_AUUCAUUA-AUUCCUCC |
| | GUUGAGUGGC | | ACTGTCGTTGAGTG | 0_1-1_mismatch_A-C |
| | | | GC | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric AAGG-GGAA |
| | | | | 38_1-1_mismatch_G-G |
| | | | | 43_1-1_wobble_U-G |
| | | | | 50_1-1_wobble_U-G |
| | | | | 54_1-1_wobble_G-U |
| 401 | CUCAGUAGCGGCCAC | 735 | CTCAGTAGCGGCCA | -36_1-1_wobble_U-G |
| | AGCUCCGUGGAAGGC | | CAGCTCCGTGGAAG | -33_1-1_wobble_G-U |
| | UUUUGGAAGUUCUUU | | GCTTTTGGAAGTTC | -23_1-1_wobble_U-G |
| | UAUGAGUACGUCUAC | | TTTTATGAGTACGT | -21_1-1_wobble_G-U |
| | GGCAUUCCUCCUCCU | | CTACGGCATTCCTC | -18_1-1_wobble_U-G |
| | UUGCAUCGCACUGUC | | CTCCTTTGCATCGC | -4_8-8_internal_loop- |
| | GUUGAGUGGC | | ACTGTCGTTGAGTG | symmetric_AUUCAUUA-AUUCCUCC |
| | | | GC | 0_1-1_mismatch A-C |
| | | | | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric_AAGG-GGAA |
| | | | | 38_1-1_mismatch_G-G |
| | | | | 43_1-1_wobble_U-G |
| | | | | 50_1-1_wobble_U-G |
| | | | | 54_1-1_wobble_G-U |
| 402 | CUCAGUAGCGGCCAC | 736 | CTCAGTAGCGGCCA | -36_1-1_wobble_U-G |
| | AGCUCCGUGGAAGGC | | CAGCTCCGTGGAAG | -33_1-1_wobble_G-U |
| | UUUUGGAAGUUCUUU | | GCTTTTGGAAGTTC | -23_1-1_wobble_U-G |
| | UAUGAGUACGUCUAC | | TTTTATGAGTACGT | -21_1-1_wobble_G-U |
| | GGCAUUCCUUCUCCU | | CTACGGCATTCCTT | -18_wobble_U-G |
| | UUGCAUCGCACUGUC | | CTCCTTTGCATCGC | -4_8-8_internal_loop- |
| | GUUGAGUGGC | | ACTGTCGTTGAGTG | symmetric_AUUCAUUA-AUUCCUUC |
| | | | GC | 0_1-1_mismatch A-C |
| | | | | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric_AAGG-GGAA |
| | | | | 38_1-1_mismatch_G-G |
| | | | | 43_1-1_wobble_U-G |
| | | | | 50_1-1_wobble_U-G |
| | | | | 54_1-1_wobble_G-U |
| 403 | CUCAGUAGCGGCCAC | 737 | CTCAGTAGCGGCCA | -36_1-1_wobble_U-G |
| | AGCUCCGUGGUAGGC | | CAGCTCCGTGGTAG | -33_1-1_wobble_G-U |
| | UUUUGGAAGUUCUUU | | GCTTTTGGAAGTTC | -23_wobble_U-G |
| | UAUGAGUACGUCUAC | | TTTTATGAGTACGT | -21_wobble_G-U |
| | GGCAUUCCUUCUCCU | | CTACGGCATTCCTT | -18_wobble_U-G |
| | UUGCAUCGCACUGUC | | CTCCTTTGCATCGC | -4_8-8_internal_loop- |
| | GUUGAGUGGC | | ACTGTCGTTGAGTG | symmetric_AUUCAUUA-AUUCCUUC |
| | | | GC | 0_1-1_mismatch_A-C |
| | | | | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4 bulge-symmetric_AAGG-GGUA |
| | | | | 38_1-1_mismatch_G-G |
| | | | | 43_1-1_wobble_U-G |
| | | | | 50_1-1_wobble_U-G |
| | | | | 54_1-1_wobble_G-U |
| 404 | CUUAGUAGCGGCCAC | 738 | CTTAGTAGCGGCCA | -36_1-1_wobble_U-G |
| | AGCUCCGUGGUAGGC | | CAGCTCCGTGGTAG | -33_1-1_wobble_G-U |
| | UUUUGGAAGUUCUUU | | GCTTTTGGAAGTTC | -23_1-1_wobble_U-G |
| | UAUGAGUACGUCUAC | | TTTTATGAGTACGT | -21_1-1_wobble_G-U |
| | GGCAUUCCUUCUCCU | | CTACGGCATTCCTT | -18_1-1_wobble_U-G |
| | UUGCAUCGCACUGUC | | CTCCTTTGCATCGC | -4_8-8_internal_loop- |
| | GUUGAGUGGC | | ACTGTCGTTGAGTG | symmetric_AUUCAUUA-AUUCCUUC |
| | | | GC | 0_1-1_mismatch_A-C |
| | | | | 2_1-1_wobble_G-U |
| | | | | 5_1-1_wobble_U-G |
| | | | | 9_1-1_wobble_U-G |
| | | | | 14_1-1_wobble_G-U |
| | | | | 19_1-1_wobble_G-U |
| | | | | 24_1-1_wobble_U-G |
| | | | | 29_1-1_wobble_G-U |
| | | | | 33_4-4_bulge-symmetric_AAGG-GGUA |
| | | | | 38_1-1_mismatch_G-G |
| | | | | 43_1-1_wobble_U-G |
| | | | | 50_1-1_wobble_U-G |
| | | | | 54_1-1_wobble_G-U |
| | | | | 57_1-1_wobble_G-U |
| 405 | CUCAGCAGCAGCCAC | 739 | CTCAGCAGCAGCCA | -9_3-3_bulge-symmetric_AUU-UCA |
| | AACUCCGUGGACGGC | | CAACTCCGTGGACG | -6_2-2_bulge-symmetric_AU-CG |
| | CUUUGAAAGUCCUUU | | GCCTTTGAAAGTCC | 0_1-1_mismatch A-C |
| | CAUGAAUAGUUCCAC | | TTTCATGAATAGTT | 5_2-2_bulge-symmetric_UG-GU |
| | GGCUACGGUCAUCCU | | CCACGGCTACGGTC | 33_4-4_bulge-symmetric_AAGG-GGAC |
| | UUACACCACACUGUC | | ATCCTTTACACCAC | 38_1-1_mismatch_G-G |
| | GUCGAAUGGC | | ACTGTCGTCGAATG | |
| | | | GC | |
| 406 | CUCAGCAGCAGUCAC | 740 | CTCAGCAGCAGTCA | -35_1-1_wobble_U-G |
| | GACUCCGUGGUAGGC | | CGACTCCGTGGTAG | -21_1-1_wobble_G-U |
| | CUUUGAGAGUCCUUU | | GCCTTTGAGAGTCC | -9 3-3_bulge-symmetric_AUU-UCA |
| | CGUGAAUAGUUCCAC | | TTTCGTGAATAGTT | -6_2-2_bulge-symmetric_AU-CG |
| | GGCUACGGUCAUCCU | | CCACGGCTACGGTC | 0_1-1_mismatch A-C |
| | UUACAUCACACUGUC | | ATCCTTTACATCAC | 5_2-2_bulge-symmetric_UG-GU |
| | GUCGGAUGGC | | ACTGTCGTCGGATG | 13_1-1_wobble_U-G |
| | | | GC | 23_1-1_wobble_U-G |
| | | | | 33_4-4_bulge-symmetric_AAGG-GGUA |
| | | | | 38_1-1 mismatch_G-G |
| | | | | 44_1-1_wobble_U-G |
| | | | | 48_1-1_wobble_G-U |
| 407 | CUCAGCAGCAGUCAC | 741 | CTCAGCAGCAGTCA | -35_1-1_wobble_U-G |
| | GACUCCGUGGACGGC | | CGACTCCGTGGACG | -21_1-1_wobble_G-U |
| | CUUUGAGAGUCCUUU | | GCCTTTGAGAGTCC | -9_3-3_bulge-symmetric_AUU-UCA |
| | CGUGAAUAGUUCCAC | | TTTCGTGAATAGTT | -6_2-2_bulge-symmetric_AU-CG |
| | GGCUACGGUCAUCCU | | CCACGGCTACGGTC | 0_1-1_mismatch A-C |
| | UUACAUCACACUGUC | | ATCCTTTACATCAC | 5_2-2_bulge-symmetric_UG-GU |
| | GUCGGAUGGC | | ACTGTCGTCGGATG | 13_1-1_wobble_U-G |
| | | | GC | 23_1-1_wobble_U-G |
| | | | | 33_4-4_bulge-symmetric_AAGG-GGAC |
| | | | | 38_1-1_mismatch_G-G |
| | | | | 44_1-1_wobble_U-G |
| | | | | 48_1-1_wobble_G-U |
| 408 | CUCAGCAGCAGUCAC | 742 | CTCAGCAGCAGTCA | -35__wobble_U-G |
| | AACUCCGUGGACGGC | | CAACTCCGTGGACG | -30_1-1_wobble_G-U |
| | CUUUGAGAGUUCUUU | | GCCTTTGAGAGTTC | -21_wobble_G-U |
| | CGUGAAUAGUUCCAC | | TTTCGTGAATAGTT | -9_3-3_bulge-symmetric_AUU-UCA |
| | GGCUACGGUCAUCCU | | CCACGGCTACGGTC | -6_2-2_bulge-symmetric_AU-CG |
| | UUACAUCACACUGUU | | ATCCTTTACATCAC | 0_1-1_mismatch A-C |
| | GUCGGAUGGC | | ACTGTTGTCGGATG | 5_2-2_bulge-symmetric_UG-GU |
| | | | GC | 13_1-1_wobble_U-G |
| | | | | 19_1-1_wobble_G-U |
| | | | | 23_1-1_wobble_U-G |
| | | | | 33_4-4_bulge-symmetric AAGG-GGAC |
| | | | | 38_1-1_mismatch_G-G |
| | | | | 48_1-1_wobble_G-U |
| 409 | CUCAGCAGCAGUCAC | 743 | CTCAGCAGCAGTCA | -35_1-1_wobble_U-G |
| | GACUCCGUGGACGGC | | CGACTCCGTGGACG | -30_1-1_wobble_G-U |
| | CUUUGAGAGUUCUUU | | GCCTTTGAGAGTTC | -21_1-1_wobble_G-U |
| | CGUGAAUAGUUCCAC | | TTTCGTGAATAGTT | -9_3-3_bulge-symmetric_AUU-UCA |
| | GGCUACGGUCAUCCU | | CCACGGCTACGGTC | -6_2-2_bulge-symmetric_AU-CG |
| | UUACAUCACACUGUU | | ATCCTTTACATCAC | 0_1-1_mismatch_A-C |
| | GUCGGAUGGC | | ACTGTTGTCGGATG | 5 2-2_bulge-symmetric_UG-GU |
| | | | GC | 13_1-1_wobble_U-G |
| | | | | 19_1-1_wobble_G-U |
| | | | | 23_1-1_wobble_U-G |
| | | | | 33 4-4_bulge-symmetric_AAGG-GGAC |
| | | | | 38_1-1_mismatch_G-G |
| | | | | 44_1-1_wobble_U-G |
| | | | | 48_1-1_wobble_G-U |
| 410 | CUCAGCAGCAGUCAC | 744 | CTCAGCAGCAGTCA | -35_1-1_wobble_U-G |
| | GACUCCGUGGACGGU | | CGACTCCGTGGACG | -30_1-1_wobble_G-U |
| | CUUUGAGAGUCCUUU | | GTCTTTGAGAGTCC | -25_1-1_wobble_U-G |
| | CGUGAAUAGUUCCAC | | TTTCGTGAATAGTT | -21_1-1_wobble_G-U |
| | GGCUACGGUCAUCCU | | CCACGGCTACGGTC | -9_3-3_bulge-symmetric_AUU-UCA |
| | UUACAUCACGCUGUU | | ATCCTTTACATCAC | -6_2-2_bulge-symmetric_AU-CG |
| | GUCGGAUGGC | | GCTGTTGTCGGATG | 0_1-1_mismatch A-C |
| | | | GC | 5_2-2_bulge-symmetric_UG-GU |
| | | | | 13_1-1_wobble_U-G |
| | | | | 23_1-1_wobble_U-G |
| | | | | 30_1-1_wobble_G-U |
| | | | | 33 4-4_bulge-symmetric_AAGG-GGAC |
| | | | | 38_1-1_mismatch_G-G |
| | | | | 44_1-1_wobble_U-G |
| | | | | 48_1-1_wobble_G-U |
| 411 | CUCAGCAGCAGUCAC | 745 | CTCAGCAGCAGTCA | -35_1-1_wobble_U-G |
| | GACUCCGUGGACGGU | | CGACTCCGTGGACG | -30_1-1_wobble_G-U |
| | CUUUGAGAGUUCUUU | | GTCTTTGAGAGTTC | -25_1-1_wobble_U-G |
| | CGUGAAUAGUUCCAC | | TTTCGTGAATAGTT | -21_wobble_G-U |
| | GGCUACGGUCAUCCU | | CCACGGCTACGGTC | -9_3-3_bulge-symmetric_AUU-UCA |
| | UUACAUCACGCUGUU | | ATCCTTTACATCAC | -6_2-2_bulge-symmetric_AU-CG |
| | GUCGGAUGGC | | GCTGTTGTCGGATG | 0_1-1_mismatch_A-C |
| | | | GC | 5_2-2_bulge-symmetric_UG-GU |
| | | | | 13_1-1_wobble_U-G |
| | | | | 19_1-1_wobble_G-U |
| | | | | 23_1-1_wobble_U-G |
| | | | | 30_1-1_wobble_G-U |
| | | | | 33 4-4_bulge-symmetric_AAGG-GGAC |
| | | | | 38_1-1_mismatch_G-G |
| | | | | 44_1-1_wobble_U-G |
| | | | | 48_1-1_wobble_G-U |
| 412 | CUCAGCGGCAGUCAC | 746 | CTCAGCGGCAGTCA | -40_1-1_wobble_G-U |
| | GACUCCGUGGACGGU | | CGACTCCGTGGACG | -35_1-1_wobble_U-G |
| | CUUUGAGAGUCCUUU | | GTCTTTGAGAGTCC | -30_1-1_wobble_G-U |
| | CGUGAAUAGUUCCAC | | TTTCGTGAATAGTT | -25_1-1_wobble_U-G |
| | GGCUACGGUCAUCCU | | CCACGGCTACGGTC | -21_1-1_wobble_G-U |
| | UUACAUCACGCUGUU | | ATCCTTTACATCAC | -9_3-3_bulge-symmetric_AUU-UCA |
| | GUCGGAUGGU | | GCTGTTGTCGGATG | -6_2-2_bulge-symmetric_AU-CG |
| | | | GT | 0_1-1_mismatch A-C |
| | | | | 5_2-2_bulge-symmetric_UG-GU |
| | | | | 13_1-1_wobble_U-G |
| | | | | 23_1-1_wobble_U-G |
| | | | | 30_1-1_wobble_G-U |
| | | | | 33_4-4_bulge-symmetric_AAGG-GGAC |
| | | | | 38_1-1_mismatch_G-G |
| | | | | 44_1-1_wobble_U-G |
| | | | | 48_1-1_wobble_G-U |
| | | | | 53_1-1_wobble_U-G |
| 413 | CUCAGCGGCAGUCAC | 747 | CTCAGCGGCAGTCA | -40_1-1_wobble_G-U |
| | GACUCCGUGGACGGU | | CGACTCCGTGGACG | -35_1-1_wobble_U-G |
| | CUUUGAGAGUUCUUU | | GTCTTTGAGAGTTC | -30_1-1_wobble_G-U |
| | CGUGAAUAGUUCCAC | | TTTCGTGAATAGTT | -25_1-1_wobble_U-G |
| | GGCUACGGUCAUCCU | | CCACGGCTACGGTC | -21_1-1_wobble_G-U |
| | UUACAUCACGCUGUU | | ATCCTTTACATCAC | -9_3-3_bulge-symmetric_AUU-UCA |
| | GUCGGAUGGU | | GCTGTTGTCGGATG | -6_2-2_bulge-symmetric_AU-CG |
| | | | GT | 0_1-1_mismatch A-C |
| | | | | 5_2-2_bulge-symmetric_UG-GU |
| | | | | 13_1-1_wobble_U-G |
| | | | | 19_1-1_wobble_G-U |
| | | | | 23_1-1_wobble_U-G |
| | | | | 30_1-1_wobble_G-U |
| | | | | 33_4-4_bulge-symmetric_AAGG-GGAC |
| | | | | 38_1-1_mismatch_G-G |
| | | | | 44_1-1_wobble_U-G |
| | | | | 48_1-1_wobble_G-U |
| | | | | 53_1-1_wobble_U-G |
| 414 | CAGCAGCCACAACUC | 750 | CAGCAGCCACAACT | -33_wobble_G-U |
| | CCUCAAACCGCUUUG | | CCCTCAAACCGCTT | -30_1-1_wobble_G-U |
| | AAAGUCCUUUCAUGU | | TGAAAGTCCTTTCA | -25_1-1_wobble_U-G |
| | AUAGAUCCACGGCUA | | TGTATAGATCCACG | -18_6-6_internal_loop-symmetric_ |
| | UACAAUUCCUUUUGU | | GCTATACAATTCCT | UGGUGU-UGUGGU |
| | GGUCGCUGUUGUUGG | | TTTGTGGTCGCTGT | -6_3-3_bulge-symmetric_CAU-UAC |
| | | | TGTTGG | 0_1-1_mismatch_A-C |
| | | | | 6_1-1_mismatch G-G |
| | | | | 10_1-1_mismatch_U-U |
| | | | | 30_6-6_internal_loop-symmetric_ |
| | | | | GCCAAG-AAACCG |
| 415 | CAGCAGCCACAGCUC | 751 | CAGCAGCCACAGCT | -33_1-1_wobble_G-U |
| | UCUCACACCGCUUUG | | CTCTCACACCGCTT | -30_1-1_wobble_G-U |
| | GAAGUUCUUUCGUGC | | TGGAAGTTCTTTCG | -25_1-1_wobble_U-G |
| | AUGGAUCCACGGCUA | | TGCATGGATCCACG | -18_6-6_internal_loop-symmetric_ |
| | UACAAUUUCUUUUGU | | GCTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | 0_1-1_mismatch A-C |
| | | | | 6_1-1_mismatch_G-G |
| | | | | 7_1-1_wobble_U-G |
| | | | | 10_1-1_mismatch U-C |
| | | | | 13_1-1_wobble_U-G |
| | | | | 19_1-1_wobble_G-U |
| | | | | 24_1-1_wobble_U-G |
| | | | | 30_6-6_internal_loop-symmetric_ |
| | | | | GCCAAG-ACACCG |
| | | | | 39_1-1_wobble_G-U |
| | | | | 43_1-1_wobble_U-G |
| 416 | CAGCAGCCACAGCUC | 752 | CAGCAGCCACAGCT | -33_1-1_wobble_G-U |
| | UCUCAAACCGCUUUG | | CTCTCAAACCGCTT | -30_1-1_wobble_G-U |
| | GAAGUUCUUUCGUGC | | TGGAAGTTCTTTCG | -25_1-1_wobble_U-G |
| | AUGGAUCCACGGCUA | | TGCATGGATCCACG | -18_6-6_internal_loop-symmetric_ |
| | UACAAUUUCUUUUGU | | GCTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | 0_1-1_mismatch A-C |
| | | | | 6_1-1_mismatch G-G |
| | | | | 7_1-1_wobble_U-G |
| | | | | 10_1-1_mismatch U-C |
| | | | | 13_1-1_wobble_U-G |
| | | | | 19_1-1_wobble_G-U |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| 417 | CAGCAGCCACAGCUC | 753 | CAGCAGCCACAGCT | -33_1-1_wobble_G-U |
| | UCUCAAACCGCUUUG | | CTCTCAAACCGCTT | -30_1-1_wobble_G-U |
| | GAGGUUCUUUCGUGC | | TGGAGGTTCTTTCG | -25_1-1_wobble_U-G |
| | AUGGAUCCACGGCUA | | TGCATGGATCCACG | -18_6-6_internal_loop-symmetric_ |
| | UACAAUUUCUUUUGU | | GCTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | 0_1-1_mismatch A-C |
| | | | | 6_1-1 mismatch_G-G |
| | | | | 7_1-1_wobble_U-G |
| | | | | 10_1-1_mismatch_U-C |
| | | | | 13_1-1_wobble_U-G |
| | | | | 19_1-1_wobble_G-U |
| | | | | 22_1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| 418 | CAGUAGCCACAGCUC | 754 | CAGTAGCCACAGCT | -33_1-1_wobble_G-U |
| | UCUCAAACCGCUUUG | | CTCTCAAACCGCTT | -30_1-1_wobble_G-U |
| | GAGGUUCUUUCGUGC | | TGGAGGTTCTTTCG | -25_1-1_wobble_U-G |
| | AUGGAUCCACGGCUA | | TGCATGGATCCACG | -18_6-6_internal_loop-symmetric_ |
| | UACAAUUUCUUUUGU | | GCTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | 0_1-1_mismatch A-C |
| | | | | 6_1-1_mismatch_G-G |
| | | | | 7_1-1_wobble_U-G |
| | | | | 10_1-1_mismatch_U-C |
| | | | | 13_1-1_wobble_U-G |
| | | | | 19_1-1_wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 51_1-1_wobble_G-U |
| 419 | CAGUAGCCACAGCUC | 755 | CAGTAGCCACAGCT | -33_1-1_wobble_G-U |
| | UCUCAAACCGCUUUG | | CTCTCAAACCGCTT | -30_1-1_wobble_G-U |
| | GAGGUUCUUUCGUGC | | TGGAGGTTCTTTCG | -25_1-1_wobble_U-G |
| | AUGGAUCCACGGUUA | | TGCATGGATCCACG | -18_6-6_internal_loop-symmetric_ |
| | UACAAUUUCUUUUGU | | GTTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | -3_1-1_wobble_G-U |
| | | | | 0_1-1_mismatch_A-C |
| | | | | 6_1-1_mismatch_G-G |
| | | | | 7_1-1_wobble_U-G |
| | | | | 10_1-1_mismatch_U-C |
| | | | | 13_1-1_wobble_U-G |
| | | | | 19_1-1_wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 51_1-1_wobble_G-U |
| 420 | CAGUAGCCACAGCUC | 756 | CAGTAGCCACAGCT | -33_1-1_wobble_G-U |
| | UCUCAAACCGCUUUG | | CTCTCAAACCGCTT | -30_wobble_G-U |
| | GAGGUUCUUUCGUGC | | TGGAGGTTCTTTCG | -25_1-1_wobble_U-G |
| | AUGGAUUCACGGUUA | | TGCATGGATTCACG | -18_6-6_internal_loop-symmetric_ |
| | UACAAUUUCUUUUGU | | GTTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | -3_1-1_wobble_G-U |
| | | | | 0_1-1_mismatch_A-C |
| | | | | 3_1-1_wobble_G-U |
| | | | | 6_1-1_mismatch_G-G |
| | | | | 7_1-1_wobble_U-G |
| | | | | 10_1-1_mismatch_U-C |
| | | | | 13_wobble_U-G |
| | | | | 19_1-1_wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 51_1-1_wobble_G-U |
| 421 | CAGUAGCCACAGCUC | 757 | CAGTAGCCACAGCT | -33_1-1_wobble_G-U |
| | UCUCAAACCGCUUUG | | CTCTCAAACCGCTT | -30_1-1_wobble_G-U |
| | GAGGUUCUUUCGUGU | | TGGAGGTTCTTTCG | -25_1-1_wobble_U-G |
| | AUGGAUUCACGGUUA | | TGTATGGATTCACG | -18_6-6_internal_loop-symmetric_ |
| | UACAAUUUCUUUUGU | | GTTATACAATTTCT | UGGUGU-UGUGGU |
| | GGUCGCUGUUGUUGA | | TTTGTGGTCGCTGT | -13_1-1_wobble_G-U |
| | | | TGTTGA | -6_3-3_bulge-symmetric_CAU-UAC |
| | | | | -3_1-1_wobble_G-U |
| | | | | 0_1-1_mismatch_A-C |
| | | | | 3_1-1_wobble_G-U |
| | | | | 6_1-1_mismatch_G-G |
| | | | | 7_1-1_wobble_U-G |
| | | | | 10_1-1_mismatch_U-U |
| | | | | 13_1-1_wobble_U-G |
| | | | | 19_1-1_wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 51_1-1_wobble_G-U |
| 422 | CAGUAGCCACAGCUC | 758 | CAGTAGCCACAGCT | -33_1-1_wobble_G-U |
| | UCUCAAACCGCUUUG | | CTCTCAAACCGCTT | -30_1-1_wobble_G-U |
| | GAGGUUCUUUCGUG | | TGGAGGTTCTTTCG | -25_1-1_wobble_U-G |
| | UAUGGAUUCACGGU | | TGTATGGATTCACG | -18_6-6_internal_loop-symmetric_ |
| | UAUACAAUUUCUUU | | GTTATACAATTTCT | UGGUGU-UGUGGU |
| | UGUGGUCGCUGUUG | | TTTGTGGTCGCTGT | -13_1-1_wobble_G-U |
| | UUGG | | TGTTGG | -6_3-3_bulge-symmetric_CAU-UAC |
| | | | | -3_1-1_wobble_G-U |
| | | | | 0_1-1_mismatch_A-C |
| | | | | 3_1-1_wobble_G-U |
| | | | | 6_1-1_mismatch G-G |
| | | | | 7_1-1_wobble_U-G |
| | | | | 10_1-1 mismatch_U-U |
| | | | | 13_1-1_wobble_U-G |
| | | | | 19__wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 51_1-1_wobble_G-U |
| 423 | CAGUAGCUACAGCUC | 759 | CAGTAGCTACAGCT | -33_1-1_wobble_G-U |
| | UCUCAAACCGCUUUG | | CTCTCAAACCGCTT | -30_1-1_wobble_G-U |
| | GAGGUUCUUUCGUG | | TGGAGGTTCTTTCG | -25_1-1_wobble_U-G |
| | UAUGGAUUCACGGU | | TGTATGGATTCACG | -18_6-6_internal_loop-symmetric_ |
| | UAUACAAUUUCUUU | | GTTATACAATTTCT | UGGUGU-UGUGGU |
| | UGUGGUCGCUGUUG | | TTTGTGGTCGCTGT | -13_1-1_wobble_G-U |
| | UUGG | | TGTTGG | -6_3-3_bulge-symmetric_CAU-UAC |
| | | | | -3_1-1_wobble_G-U |
| | | | | 0_1-1_mismatch A-C |
| | | | | 3_1-1_wobble_G-U |
| | | | | 6_1-1_mismatch G-G |
| | | | | 7_1-1_wobble_U-G |
| | | | | 10_1-1_mismatch U-U |
| | | | | 13_1-1_wobble_U-G |
| | | | | 19_1-1_wobble_G-U |
| | | | | 22_1-1_wobble_U-G |
| | | | | 24_1-1_wobble_U-G |
| | | | | 30_6-6_internal_loop-symmetric_ |
| | | | | GCCAAG-AAACCG |
| | | | | 39_1-1_wobble_G-U |
| | | | | 43_1-1_wobble_U-G |
| | | | | 47_1-1_wobble_G-U |
| | | | | 51_1-1_wobble_G-U |
| 424 | CAGCAGCCACAACUC | 760 | CAGCAGCCACAACT | -30_1-1_wobble_G-U |
| | CCUCCUACAAACUUG | | CCCTCCTACAAACT | -10_6-6_internal_loop-symmetric_ |
| | AAAGUCCUUUCAUG | | TGAAAGTCCTTTCA | AGGAAU-CGGAGA |
| | AAUACAACCACGGCU | | TGAATACAACCACG | -6_0-1_bulge-asymmetric_-U |
| | AAUUGACGGAGAUU | | GCTAATTGACGGAG | 0_1-1_mismatch A-C |
| | ACACCACACUGUU | | ATTACACCACACTG | 4_1-1_mismatch_A-A |
| | | | TT | 28_6-6_internal_loop-symmetric_ |
| | | | | AGGCCA-ACAAAC |
| 425 | CAGCAGCCACGACUC | 761 | CAGCAGCCACGACT | -30_1-1_wobble_G-U |
| | CCUUCUCCAAGAUUG | | CCCTTCTCCAAGAT | -25_1-1_wobble_U-G |
| | AGAGUUCUUUUAUG | | TGAGAGTTCTTTTA | -20_1-1_wobble_U-G |
| | AGUACACCCACGGCU | | TGAGTACACCCACG | -18_1-1_wobble_U-G |
| | AGUUGAUGAGGAUU | | GCTAGTTGATGAGG | -10_6-6_internal_loop-symmetric_ |
| | GCGCCACGCUGUU | | ATTGCGCCACGCTG | AGGAAU-UGAGGA |
| | | | TT | -6_0-1_bulge-asymmetric_-U |
| | | | | -6_1-1_wobble_U-G |
| | | | | 0_1-1_mismatch A-C |
| | | | | 4_1-1_mismatch_A-C |
| | | | | 9_1-1_wobble_U-G |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 44_1-1_wobble_U-G |
| 426 | CAGCAGCCACGACUC | 762 | CAGCAGCCACGACT | -30_1-1_wobble_G-U |
| | CCUUCUCCAAGAUUG | | CCCTTCTCCAAGAT | -25_1-1_wobble_U-G |
| | AGAGUUCUUUUAUG | | TGAGAGTTCTTTTA | -22_1-1_wobble_G-U |
| | AGUACACCCACGGCU | | TGAGTACACCCACG | -20_1-1_wobble_U-G |
| | AGUUGAUGAGGAUU | | GCTAGTTGATGAGG | -18_1-1_wobble_U-G |
| | GCGCUACGCUGUU | | ATTGCGCTACGCTG | -10_6-6_internal_loop-symmetric_ |
| | | | TT | AGGAAU-UGAGGA |
| | | | | -6_0-1_bulge-asymmetric_-U |
| | | | | -6_1-1_wobble_U-G |
| | | | | 0_1-1_mismatch A-C |
| | | | | 4_1-1_mismatch_A-C |
| | | | | 9_1-1_wobble_U-G |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 44_1-1_wobble_U-G |
| 427 | CAGCAGUCACGACUC | 763 | CAGCAGTCACGACT | -30_1-1_wobble_G-U |
| | CCUUCUCCAAGAUUG | | CCCTTCTCCAAGAT | -25_1-1_wobble_U-G |
| | AGAGUUCUUUUAUG | | TGAGAGTTCTTTTA | -22_1-1_wobble_G-U |
| | AGUACACCCACGGCU | | TGAGTACACCCACG | -20_1-1_wobble_U-G |
| | AGUUGAUGAGGAUU | | GCTAGTTGATGAGG | -18_1-1_wobble_U-G |
| | GCGCUACGCUGUU | | ATTGCGCTACGCTG | -10_6-6_internal_loop-symmetric_ |
| | | | TT | AGGAAU-UGAGGA |
| | | | | -6_0-1_bulge-asymmetric_-U |
| | | | | -6_1-1_wobble_U-G |
| | | | | 0_1-1_mismatch A-C |
| | | | | 4_1-1_mismatch_A-C |
| | | | | 9_1-1_wobble_U-G |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 44_1-1_wobble_U-G |
| | | | | 48_1-1_wobble_G-U |
| 428 | CAGCAGUCACGACUC | 764 | CAGCAGTCACGACT | -30_1-1_wobble_G-U |
| | CCUUCUCCAAGAUUG | | CCCTTCTCCAAGAT | -25_wobble_U-G |
| | AGAGUUCUUUUAUA | | TGAGAGTTCTTTTA | -22_wobble_G-U |
| | AGUACACCCACGGCU | | TAAGTACACCCACG | -20_wobble_U-G |
| | AGUUGAUGAGGAUU | | GCTAGTTGATGAGG | -18_1-1_wobble_U-G |
| | GCGCUACGCUGUU | | ATTGCGCTACGCTG | -10_6-6_internal_loop-symmetric_ |
| | | | TT | AGGAAU-UGAGGA |
| | | | | -6_0-1_bulge-asymmetric_-U |
| | | | | -6_1-1_wobble_U-G |
| | | | | 0_1-1_mismatch A-C |
| | | | | 4_1-1_mismatch A-C |
| | | | | 9_1-1_wobble_U-G |
| | | | | 11_1-1_mismatch_C-A |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 44_1-1_wobble_U-G |
| | | | | 48_1-1_wobble_G-U |
| 429 | CAGCAGUCACGACUC | 765 | CAGCAGTCACGACT | -30_1-1_wobble_G-U |
| | UCUUCUCCAAGAUUG | | CTCTTCTCCAAGAT | -25_1-1_wobble_U-G |
| | AGAGUUCUUUUAUA | | TGAGAGTTCTTTTA | -22_wobble_G-U |
| | AGUACACCCACGGCU | | TAAGTACACCCACG | -20_1-1_wobble_U-G |
| | AGUUGAUGAGGAUU | | GCTAGTTGATGAGG | -18_1-1_wobble_U-G |
| | GCGCUACGCUGUU | | ATTGCGCTACGCTG | -10_6-6_internal_loop-symmetric_ |
| | | | TT | AGGAAU-UGAGGA |
| | | | | -6_0-1_bulge-asymmetric_-U |
| | | | | -6_1-1_wobble_U-G |
| | | | | 0_1-1_mismatch A-C |
| | | | | 4_1-1_mismatch A-C |
| | | | | 9_1-1_wobble_U-G |
| | | | | 11_1-1_mismatch_C-A |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 39_1-1_wobble_G-U |
| | | | | 44_1-1_wobble_U-G |
| | | | | 48_1-1_wobble_G-U |
| 430 | CAGCAGUCACGACUC | 766 | CAGCAGTCACGACT | -30_1-1_wobble_G-U |
| | UCUUCUCCAAGAUUG | | CTCTTCTCCAAGAT | -25_wobble_U-G |
| | AGAGUUCUUUUAUA | | TGAGAGTTCTTTTA | -22_wobble_G-U |
| | AGUACACCCACGGCU | | TAAGTACACCCACG | -20_1-1_wobble_U-G |
| | AGUUGAUAAGGAUU | | GCTAGTTGATAAGG | -18_1-1_wobble_U-G |
| | GCGCUACGCUGUU | | ATTGCGCTACGCTG | -10_6-6_internal_loop-symmetric_ |
| | | | TT | AGGAAU-UAAGGA |
| | | | | -6_0-1_bulge-asymmetric_-U |
| | | | | -6_1-1_wobble_U-G |
| | | | | 0_1-1_mismatch_A-C |
| | | | | 4_1-1_mismatch_A-C |
| | | | | 9_1-1_wobble_U-G |
| | | | | 11_1-1_mismatch_C-A |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 39_1-1_wobble_G-U |
| | | | | 44_1-1_wobble_U-G |
| | | | | 48_1-1_wobble_G-U |
| 431 | CAGUAGUCACGACUC | 767 | CAGTAGTCACGACT | -30_1-1_wobble_G-U |
| | UCUUCUCCAAGAUUG | | CTCTTCTCCAAGAT | -25_1-1_wobble_U-G |
| | AGAGUUCUUUUAUA | | TGAGAGTTCTTTTA | -22_1-1_wobble_G-U |
| | AGUACACCCACGGCU | | TAAGTACACCCACG | -20_1-1_wobble_U-G |
| | AGUUGAUAAGGAUU | | GCTAGTTGATAAGG | -18_1-1_wobble_U-G |
| | GCGCUACGCUGUU | | ATTGCGCTACGCTG | -10_6-6_internal_loop-symmetric_ |
| | | | TT | AGGAAU-UAAGGA |
| | | | | -6_0-1_bulge-asymmetric_-U |
| | | | | -6_1-1_wobble_U-G |
| | | | | 0_1-1_mismatch A-C |
| | | | | 4_1-1_mismatch_A-C |
| | | | | 9_1-1_wobble_U-G |
| | | | | 11_1-1_mismatch_C-A |
| | | | | 14_1-1_wobble_G-U |
| | | | | 19_1-1_wobble_G-U |
| | | | | 23_1-1_wobble_U-G |
| | | | | 28_6-6_internal_loop-symmetric_ |
| | | | | AGGCCA-CCAAGA |
| | | | | 36_1-1_wobble_G-U |
| | | | | 39_1-1_wobble_G-U |
| | | | | 44_1-1_wobble_U-G |
| | | | | 48_1-1_wobble_G-U |
| | | | | 51_1-1_wobble_G-U |
| 432 | CUCAGCGGCAGUCAC | 768 | CTCAGCGGCAGTCA | -35_1-1_wobble_U-G |
| | AGCUCCUUCCUACAU | | CAGCTCCTTCCTAC | -30_1-1_wobble_G-U |
| | GAUUGGAAGUCUUU | | ATGATTGGAAGTCT | -25_1-1_wobble_U-G |
| | UUAUGGAUACGUCC | | TTTTATGGATACGT | -21_1-1_wobble_G-U |
| | ACGGCAUGAUAUGG | | CCACGGCATGATAT | -18_wobble_U-G |
| | GUUGCAUCACGCUGU | | GGGTTGCATCACGC | -10_6-6_internal_loop-symmetric_ |
| | UGUCGGAUGGC | | TGTTGTCGGATGGC | AGGAAU-UAUGGG |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 433 | CUCAGCGGCAGUCAC | 769 | CTCAGCGGCAGTCA | -40_1-1_wobble_G-U |
| | AGCUCCUUCCUACAU | | CAGCTCCTTCCTAC | -35_1-1_wobble_U-G |
| | GAUUGGAAGUCUUU | | ATGATTGGAAGTCT | -30_wobble_G-U |
| | UUAUGGAUACGUCC | | TTTTATGGATACGT | -25_1-1_wobble_U-G |
| | ACGGCAUGAUAUGG | | CCACGGCATGATAT | -21_wobble_G-U |
| | GUUGCAUCACGCUGU | | GGGTTGCATCACGC | -18_wobble_U-G |
| | UGUCGGAUGGU | | TGTTGTCGGATGGT | -10_6-6_internal_loop-symmetric_ |
| | | | | AGGAAU-UAUGGG |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 434 | CUCAGCGGCAGUCAC | 770 | CTCAGCGGCAGTCA | -40_1-1_wobble_G-U |
| | AGCUCCUUCCUACAU | | CAGCTCCTTCCTAC | -35_1-1_wobble_U-G |
| | GAUUGGAAGUCUUU | | ATGATTGGAAGTCT | -30_1-1_wobble_G-U |
| | UUAUGGAUACGUCC | | TTTTATGGATACGT | -25_1-1_wobble_U-G |
| | ACGGCAUGAUAUGG | | CCACGGCATGATAT | -21_1-1_wobble_G-U |
| | AUUGCAUCACGCUGU | | GGATTGCATCACGC | -18_1-1_wobble_U-G |
| | UGUCGGAUGGU | | TGTTGTCGGATGGT | -10_6-6_internal_loop-symmetric_ |
| | | | | AGGAAU-UAUGGA |
| | | | | -4_2-0_bulge-asymmetric_UA- |
| | | | | 0_1-1_mismatch_A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 435 | CUCAGCGGCAGUCAC | 771 | CTCAGCGGCAGTCA | -40_1-1_wobble_G-U |
| | AGCUCCUUCCUACAU | | CAGCTCCTTCCTAC | -35_wobble_U-G |
| | GAUUGGAAGUCUUU | | ATGATTGGAAGTCT | -33_1-1_wobble_G-U |
| | UUAUGGAUACGUCC | | TTTTATGGATACGT | -30_1-1_wobble_G-U |
| | ACGGCAUGAUAUGG | | CCACGGCATGATAT | -25_1-1_wobble_U-G |
| | AUUGCAUCACGCUGU | | GGATTGCATCACGC | -21_1-1_wobble_G-U |
| | UGUUGGAUGGU | | TGTTGTTGGATGGT | -18_1-1_wobble_U-G |
| | | | | -10_6-6_internal_loop-symmetric_ |
| | | | | AGGAAU-UAUGGA |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 436 | CUCAGCGGCAGUCAC | 772 | CTCAGCGGCAGTCA | -40_1-1_wobble_G-U |
| | AGCUCCUUCCUACAU | | CAGCTCCTTCCTAC | -35_1-1_wobble_U-G |
| | GAUUGGAAGUCUUU | | ATGATTGGAAGTCT | -33_1-1_wobble_G-U |
| | UUAUGGAUACGUCC | | TTTTATGGATACGT | -30_1-1_wobble_G-U |
| | ACGGCAUGAUAUGG | | CCACGGCATGATAT | -25_1-1_wobble_U-G |
| | AUUGCAUCGCGCUGU | | GGATTGCATCGCGC | -23_1-1_wobble_U-G |
| | UGUUGGAUGGU | | TGTTGTTGGATGGT | -21_1-1_wobble_G-U |
| | | | | -18_1-1_wobble_U-G |
| | | | | -10_6-6_internal_loop-symmetric_ |
| | | | | AGGAAU-UAUGGA |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_wobble_G-U |
| | | | | 53_1-1_wobble_U-G |
| 437 | CUCAGCGGCAGUCAC | 773 | CTCAGCGGCAGTCA | -40_1-1_wobble_G-U |
| | AGCUCCUUCCUACAU | | CAGCTCCTTCCTAC | -35_1-1_wobble_U-G |
| | GAUUGGAAGUCUUU | | ATGATTGGAAGTCT | -33_1-1_wobble_G-U |
| | UUAUGGAUACGUCC | | TTTTATGGATACGT | -30_1-1_wobble_G-U |
| | ACGGCAUGAUAAGG | | CCACGGCATGATAA | -25_1-1_wobble_U-G |
| | AUUGCAUCGCGCUGU | | GGATTGCATCGCGC | -23_1-1_wobble_U-G |
| | UGUUGGAUGGU | | TGTTGTTGGATGGT | -21_1-1_wobble_G-U |
| | | | | -18_1-1_wobble_U-G |
| | | | | -10_6-6_internal_loop-symmetric_ |
| | | | | AGGAAU-UAAGGA |
| | | | | -4_2-0_bulge-asymmetric_UA- |
| | | | | 0_1-1_mismatch_A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 438 | CUCAGCGGCAGUCAC | 774 | CTCAGCGGCAGTCA | -40_1-1_wobble_G-U |
| | AGCUCCUUCCUACAU | | CAGCTCCTTCCTAC | -35_1-1_wobble_U-G |
| | GAUUGGAGGUCUUU | | ATGATTGGAGGTCT | -33_1-1_wobble_G-U |
| | UUAUGGAUACGUCC | | TTTTATGGATACGT | -30_1-1_wobble_G-U |
| | ACGGCAUGAUAAGG | | CCACGGCATGATAA | -25_wobble_U-G |
| | AUUGCAUCGCGCUGU | | GGATTGCATCGCGC | -23_1-1_wobble_U-G |
| | UGUUGGAUGGU | | TGTTGTTGGATGGT | -21_1-1_wobble_G-U |
| | | | | -18_1-1_wobble_U-G |
| | | | | -10_6-6_internal_loop-symmetric_ |
| | | | | AGGAAU-UAAGGA |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 22_1-1_wobble_U-G |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| 439 | CUUAGCGGCAGUCAC | 775 | CTTAGCGGCAGTCA | -40_1-1_wobble_G-U |
| | AGCUCCUUCCUACAU | | CAGCTCCTTCCTAC | -35_1-1_wobble_U-G |
| | GAUUGGAGGUCUUU | | ATGATTGGAGGTCT | -33_1-1_wobble_G-U |
| | UUAUGGAUACGUCC | | TTTTATGGATACGT | -30_1-1_wobble_G-U |
| | ACGGCAUGAUAAGG | | CCACGGCATGATAA | -25_1-1_wobble_U-G |
| | AUUGCAUCGCGCUGU | | GGATTGCATCGCGC | -23_1-1_wobble_U-G |
| | UGUUGGAUGGU | | TGTTGTTGGATGGT | -21_1-1_wobble_G-U |
| | | | | -18_1-1_wobble_U-G |
| | | | | -10_6-6_internal_loop-symmetric_ |
| | | | | AGGAAU-UAAGGA |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 22_1-1_wobble_U-G |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_1-1_wobble_U-G |
| | | | | 48_1-1_wobble_G-U |
| | | | | 53_1-1_wobble_U-G |
| | | | | 57_wobble_G-U |
| 440 | CUUAGCGGCAGUCAC | 776 | CTTAGCGGCAGTCA | -40_1-1_wobble_G-U |
| | AGCUCCUUCCUACAU | | CAGCTCCTTCCTAC | -36_1-1_wobble_U-G |
| | GAUUGGAGGUCUUU | | ATGATTGGAGGTCT | -35_1-1_wobble_U-G |
| | UUAUGGAUACGUCC | | TTTTATGGATACGT | -33_1-1_wobble_G-U |
| | ACGGCAUGAUAAGG | | CCACGGCATGATAA | -30_1-1_wobble_G-U |
| | AUUGCAUCGCGCUGU | | GGATTGCATCGCGC | -25_1-1_wobble_U-G |
| | UGUUGGGUGGU | | TGTTGTTGGGTGGT | -23_1-1_wobble_U-G |
| | | | | -21_1-1_wobble_G-U |
| | | | | -18_1-1_wobble_U-G |
| | | | | -10_6-6_internal_loop-symmetric_ |
| | | | | AGGAAU-UAAGGA |
| | | | | -4_2-0_bulge-asymmetric_UA- |
| | | | | 0_1-1_mismatch A-C |
| | | | | 5_1-1_wobble_U-G |
| | | | | 10_1-1_wobble_U-G |
| | | | | 14_1-1_wobble_G-U |
| | | | | 18_1-1_wobble_G-U |
| | | | | 22_1-1_wobble_U-G |
| | | | | 24_1-1_wobble_U-G |
| | | | | 28_6-6_internal_loop-symmetric_ |
| | | | | AGGCCA-ACAUGA |
| | | | | 38_1-1_wobble_G-U |
| | | | | 43_wobble_U-G |
| | | | | 48_wobble_G-U |
| | | | | 53_1-1_wobble_U-G |
| | | | | 57_1-1_wobble_G-U |
| 777 | CUCAGCAGCAGCCAC | 785 | CTCAGCAGCAGCCA | -40_1-1_wobble_G-U |
| | AACUCCGUGCGGGGC | | CAACTCCGTGCGGG | -6_6-6_internal_loop-symmetric_ |
| | CUUUGAAAGUCCUU | | GCCTTTGAAAGTCC | AUUCAU-UACUGG |
| | UCAUGAAUACAUCU | | TTTCATGAATACAT | 0_1-1_mismatch_A-C |
| | ACGGCUAUACUGGUC | | CTACGGCTATACTG | 2_1-1_wobble_G-U |
| | CUUUACACCACACUG | | GTCCTTTACACCAC | 33_2-2_bulge-symmetric_AA-GG |
| | UCGUCGAAUGGU | | ACTGTCGTCGAATG | 36_1-1_mismatch G-G |
| | | | GT | 38_1-1_mismatch_G-G |
| 778 | CUCAGCAGCAGCCAC | 786 | CTCAGCAGCAGCCA | -40_1-1_wobble_G-U |
| | AACUCCGUGCGGGGC | | CAACTCCGTGCGGG | -36_1-1_wobble_U-G |
| | CUUUGAAAGUCCUU | | GCCTTTGAAAGTCC | -30_1-1_wobble_G-U |
| | UCGUGAGUACGUCU | | TTTCGTGAGTACGT | -25_1-1_wobble_U-G |
| | ACGGCUAUACUGGU | | CTACGGCTATACTG | -23_1-1_wobble_U-G |
| | UCUUUGCAUCGCGCU | | GTTCTTTGCATCGC | -21_1-1_wobble_G-U |
| | GUUGUCGAGUGGU | | GCTGTTGTCGAGTG | -18_1-1_wobble_U-G |
| | | | GT | -13_1-1_wobble_G-U |
| | | | | -6_6-6_internal_loop-symmetric_ |
| | | | | AUUCAU-UACUGG |
| | | | | 0_1-1_mismatch A-C |
| | | | | 2_1-1_wobble_G-U |
| | | | | 5_1-1_wobble_U-G |
| | | | | 9_1-1_wobble_U-G |
| | | | | 13_1-1_wobble_U-G |
| | | | | 33_2-2_bulge-symmetric_AA-GG |
| | | | | 36_1-1_mismatch_G-G |
| | | | | 38_1-1_mismatch_G-G |
| 779 | CUCAGCAGCAGCCAC | 787 | CTCAGCAGCAGCCA | -40_1-1_wobble_G-U |
| | AACUCCGUGCGGGGU | | CAACTCCGTGCGGG | -36_1-1_wobble_U-G |
| | CUUUGAAAGUUCUU | | GTCTTTGAAAGTTC | -30_1-1_wobble_G-U |
| | UCGUGAGUACGUCU | | TTTCGTGAGTACGT | -25_1-1_wobble_U-G |
| | ACGGCUAUACUGGU | | CTACGGCTATACTG | -23_1-1_wobble_U-G |
| | UCUUUGCAUCGCGCU | | GTTCTTTGCATCGC | -21_1-1_wobble_G-U |
| | GUUGUCGAGUGGU | | GCTGTTGTCGAGTG | -18_1-1_wobble_U-G |
| | | | GT | -13_1-1_wobble_G-U |
| | | | | -6_6-6_internal_loop-symmetric_ |
| | | | | AUUCAU-UACUGG |
| | | | | 0_1-1_mismatch_A-C |
| | | | | 2_1-1_wobble_G-U |
| | | | | 5_1-1_wobble_U-G |
| | | | | 9_1-1_wobble_U-G |
| | | | | 13_1-1_wobble_U-G |
| | | | | 19_1-1_wobble_G-U |
| | | | | 30_1-1_wobble_G-U |
| | | | | 33 2-2_bulge-symmetric_AA-GG |
| | | | | 36_1-1_mismatch_G-G |
| | | | | 38_1-1_mismatch_G-G |
| 780 | CUCAGCAGCAGCCAC | 788 | CTCAGCAGCAGCCA | -40_1-1_wobble_G-U |
| | GACUUCGUGCGGGG | | CGACTTCGTGCGGG | -36_1-1_wobble_U-G |
| | UCUUUGAAAGUUCU | | GTCTTTGAAAGTTC | -30_1-1_wobble_G-U |
| | UUCGUGAGUACGUC | | TTTCGTGAGTACGT | -25_1-1_wobble_U-G |
| | UACGGCUAUACUGG | | CTACGGCTATACTG | -23_1-1_wobble_U-G |
| | UUCUUUGCAUCGCGC | | GTTCTTTGCATCGC | -21_wobble_G-U |
| | UGUUGUCGAGUGGU | | GCTGTTGTCGAGTG | -18_1-1_wobble_U-G |
| | | | GT | -13_1-1_wobble_G-U |
| | | | | -6_6-6_internal_loop-symmetric_ |
| | | | | AUUCAU-UACUGG |
| | | | | 0_1-1_mismatch_A-C |
| | | | | 2_1-1_wobble_G-U |
| | | | | 5_1-1_wobble_U-G |
| | | | | 9_1-1_wobble_U-G |
| | | | | 13_1-1_wobble_U-G |
| | | | | 19_1-1_wobble_G-U |
| | | | | 30_1-1_wobble_G-U |
| | | | | 33_2-2_bulge-symmetric_AA-GG |
| | | | | 36_1-1_mismatch_G-G |
| | | | | 38_1-1_mismatch_G-G |
| | | | | 40_1-1_wobble_G-U |
| | | | | 44_1-1_wobble_U-G |
| 781 | CUCAGCAGCGGCUAC | 789 | CTCAGCAGCGGCTA | -40_wobble_G-U |
| | GACUUCGUGCGGGG | | CGACTTCGTGCGGG | -36_1-1_wobble_U-G |
| | UCUUUGAAAGUUCU | | GTCTTTGAAAGTTC | -30_1-1_wobble_G-U |
| | UUCGUGAGUACGUC | | TTTCGTGAGTACGT | -25_1-1_wobble_U-G |
| | UACGGCUAUACUGG | | CTACGGCTATACTG | -23_1-1_wobble_U-G |
| | UUCUUUGCAUCGCGC | | GTTCTTTGCATCGC | -21_1-1_wobble_G-U |
| | UGUUGUCGAGUGGU | | GCTGTTGTCGAGTG | -18_1-1_wobble_U-G |
| | | | GT | -13_wobble_G-U |
| | | | | -6_6-6_internal_loop-symmetric_ |
| | | | | AUUCAU-UACUGG |
| | | | | 0_1-1_mismatch A-C |
| | | | | 2_1-1_wobble_G-U |
| | | | | 5_1-1_wobble_U-G |
| | | | | 9_1-1_wobble_U-G |
| | | | | 13_1-1_wobble_U-G |
| | | | | 19_1-1_wobble_G-U |
| | | | | 30_1-1_wobble_G-U |
| | | | | 33_2-2_bulge-symmetric_AA-GG |
| | | | | 36_1-1_mismatch_G-G |
| | | | | 38_1-1_mismatch_G-G |
| | | | | 40_1-1_wobble_G-U |
| | | | | 44_1-1_wobble_U-G |
| | | | | 47_1-1_wobble_G-U |
| | | | | 50_1-1_wobble_U-G |
| 782 | CUCGGUAGCGGCUAC | 790 | CTCGGTAGCGGCTA | -40_1-1_wobble_G-U |
| | GACUUCGUGCGGGG | | CGACTTCGTGCGGG | -36_1-1_wobble_U-G |
| | UCUUUGAAAGUUCU | | GTCTTTGAAAGTTC | -30_wobble_G-U |
| | UUCGUGAGUACGUC | | TTTCGTGAGTACGT | -25_1-1_wobble_U-G |
| | UACGGCUAUACUGG | | CTACGGCTATACTG | -23_1-1_wobble_U-G |
| | UUCUUUGCAUCGCGC | | GTTCTTTGCATCGC | -21_1-1_wobble_G-U |
| | UGUUGUCGAGUGGU | | GCTGTTGTCGAGTG | -18_1-1_wobble_U-G |
| | | | GT | -13_1-1_wobble_G-U |
| | | | | -6_6-6_internal_loop-symmetric_ |
| | | | | AUUCAU-UACUGG |
| | | | | 0_1-1_mismatch_A-C |
| | | | | 2_1-1_wobble_G-U |
| | | | | 5_1-1_wobble_U-G |
| | | | | 9_1-1_wobble_U-G |
| | | | | 13_1-1_wobble_U-G |
| | | | | 19_1-1_wobble_G-U |
| | | | | 30_1-1_wobble_G-U |
| | | | | 33_2-2_bulge-symmetric AA-GG |
| | | | | 36_1-1_mismatch_G-G |
| | | | | 38_1-1_mismatch_G-G |
| | | | | 40_1-1_wobble_G-U |
| | | | | 44_1-1_wobble_U-G |
| | | | | 47_1-1_wobble_G-U |
| | | | | 50_1-1_wobble_U-G |
| | | | | 54_1-1_wobble_G-U |
| | | | | 56_1-1_wobble_U-G |
| 783 | CUCAGCAGCAGCCAC | 791 | CTCAGCAGCAGCCA | -40_1-1_wobble_G-U |
| | AACUCCGUGCGGGGC | | CAACTCCGTGCGGG | -11_1-1_mismatch_A-G |
| | CUUUGAAAGUCCUU | | GCCTTTGAAAGTCC | -10_1-1_wobble_U-G |
| | UCAUGAAUACAUCU | | TTTCATGAATACAT | 0_1-1_mismatch_A-C |
| | ACGGCUAAUGAGGU | | CTACGGCTAATGAG | 2_1-1_wobble_G-U |
| | CCUUUACACCACACU | | GTCCTTTACACCAC | 33_2-2_bulge-symmetric_AA-GG |
| | GUCGUCGAAUGGU | | ACTGTCGTCGAATG | 36_1-1_mismatch_G-G |
| | | | GT | 38_1-1_mismatch_G-G |
| 784 | CUCAGCAGCAGCCAC | 792 | CTCAGCAGCAGCCA | -6_6-6_internal_loop-symmetric_ |
| | AACUCCCUCCUUGAU | | CAACTCCCTCCTTG | AUUCAU-UCAUAC |
| | AGGCGAAAGUCCUU | | ATAGGCGAAAGTCC | 0_1-1_mismatch_A-C |
| | UCAUGAAUAACUCCA | | TTTCATGAATAACT | 5_2-2_bulge-symmetric_UG-AC |
| | CGGCUAUCAUACUCC | | CCACGGCTATCATA | 26_6-6_internal_loop-symmetric_ |
| | UUUACACCACACUGU | | CTCCTTTACACCAC | AAAGGC-AUAGGC |
| | CGUCGAAUGGC | | ACTGTCGTCGAATG | |
| | | | GC | |

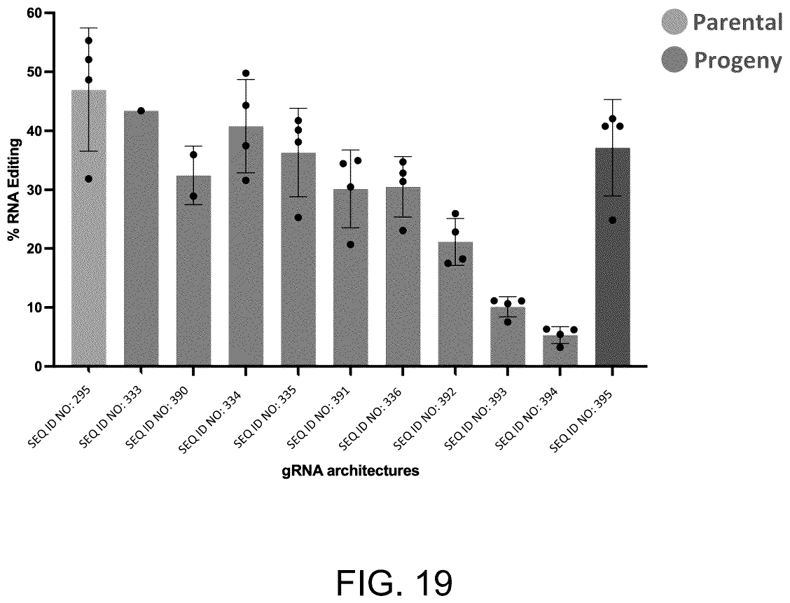

RNA editing was comparable among the RNAs in Table 10 and the parental gRNA (FIG. 19). As such, vectors can be prepared that utilize more than one guide RNA sequence in Table 1, Table 2, Table 4, Table 10, or Table 11, along with a parental gRNA.

### Example 9

### SNCA-TIS Targeting Guide RNAs Identified by Machine Learning

Select guide RNAs were identified by leveraging machine learning-based algorithms from guide RNAs trained on a cell-free high throughput screen and cell-based screening assays. The sequences of the guide RNAs are provided in Table 2 and summarized below in Table 11.

TABLE 11
Guide RNAs Identified by Machine Learning.

| | SEQ | | SEQ | |
|---|---|---|---|---|
| | ID | | ID | |
| RNA Sequence | NO: | DNA Sequence | NO: | Structural Features |
| GCCACAACUCCCUCGCGCAACUU | 341 | GCCACAACTCCCTCGCGCAACTT | 358 | -18_6-6_internal_loop-symmetric_ |
| UGAAAGUCCUUUCAUGCAUAGAU | | TGAAAGTCCTTTCATGCATAGAT | | UGGUGU-UGGAGU-5_7-7_internal_ |
| CCACGGCUCCAAAACUCCUUUUG | | CCACGGCTCCAAAACTCCTTTTG | | loop-symmetric_AUUCAUU-CCAAAAC |
| GAGUCACUGUCGUCGAAUGGCCA | | GAGTCACTGTCGTCGAATGGCCA | | 0_1-1_mismatch_A-C |
| CUCCCAGU | | CTCCCAGT | | 6_1-1_mismatch_G-G |
| | | | | 10_1-1_mismatch_U-C |
| | | | | 30_6-6_internal_loop-symmetric_ |
| | | | | GCCAAG-GCGCAA |
| GCCACAACUCCCUCGCGCAACUU | 342 | GCCACAACTCCCTCGCGCAACTT | 359 | -18_6-6_internal_loop-symmetric_ |
| UGAAAGUCCUUUCAUGCAUAGAU | | TGAAAGTCCTTTCATGCATAGAT | | UGGUGU-UGGAGU-5_4-3_bulge- |
| CCACGGCUCCAAAUUCCUUUUGG | | CCACGGCTCCAAATTCCTTTTGG | | asymmetric_CAUU-CCA |
| AGUCACUGUCGUCGAAUGGCCAC | | AGTCACTGTCGTCGAATGGCCAC | | 0_1-1_mismatch_A-C |
| UCCCAGU | | TCCCAGT | | 6_1-1_mismatch_G-G |
| | | | | 10_1-1_mismatch_U-C |
| | | | | 30_6-6_internal_loop-symmetric_ |
| | | | | GCCAAG-GCGCAA |
| GCCACAACUCCCUCGCGCAACUU | 343 | GCCACAACTCCCTCGCGCAACTT | 360 | -18_6-6_internal_loop-symmetric_ |
| UGAAAGUCCUUUCAUGCAUAGAU | | TGAAAGTCCTTTCATGCATAGAT | | UGGUGU-UGGAGU-5_4-4_bulge- |
| CCACGGCUCCAUAAUUCCUUUUG | | CCACGGCTCCATAATTCCTTTTG | | symmetric_CAUU-CCAU |
| GAGUCACUGUCGUCGAAUGGCCA | | GAGTCACTGTCGTCGAATGGCCA | | 0_1-1_mismatch_A-C |
| CUCCCAGU | | CTCCCAGT | | 6_1-1_mismatch_G-G |
| | | | | 10_1-1_mismatch_U-C |
| | | | | 30_6-6_internal_loop-symmetric_ |
| | | | | GCCAAG-GCGCAA |
| GCCACAACUCCCUCGCGCAACUU | 344 | GCCACAACTCCCTCGCGCAACTT | 361 | -18_6-6_internal_loop-symmetric_ |
| UGAAAGUCCUUUCAUGCAUAGAU | | TGAAAGTCCTTTCATGCATAGAT | | UGGUGU-UGGAGU-6_3-3_bulge- |
| CCACGGCUGCAUAAUUCCUUUUG | | CCACGGCTGCATAATTCCTTTTG | | symmetric_CAU-CAU-5_1-1_wobble_ |
| GAGUCACUGUCGUCGAAUGGCCA | | GAGTCACTGTCGTCGAATGGCCA | | U-G |
| CUCCCAGU | | CTCCCAGT | | 0_1-1_mismatch_A-C |
| | | | | 6_1-1_mismatch_G-G |
| | | | | 10_1-1_mismatch_U-C |
| | | | | 30_6-6_internal_loop-symmetric_ |
| | | | | GCCAAG-GCGCAA |
| GCCACAACUCCCUCCUACAAACU | 345 | GCCACAACTCCCTCCTACAAACT | 362 | -10_6-6_internal_loop-symmetric_ |
| UGAAAGUCCUUUCAUGAAUACAA | | TGAAAGTCCTTTCATGAATACAA | | AGGAAU-CGGAGA |
| CCACGGCUAUUGACGGAGAUUAC | | CCACGGCTATTGACGGAGATTAC | | -6_1-1_mismatch_U-U |
| ACCACACUGUCGUCGAAUGGCCA | | ACCACACTGTCGTCGAATGGCCA | | 0_1-1_mismatch_A-C |
| CUCCCAGU | | CTCCCAGT | | 4 1-1_mismatch_A-A |
| | | | | 28_6-6_internal_loop-symmetric_ |
| | | | | AGGCCA-ACAAAC |
| GCCACAACUCCCUCCUACAAACU | 346 | GCCACAACTCCCTCCTACAAACT | 363 | -10_6-6_internal_loop-symmetric_ |
| UGAAAGUCCUUUCAUGAAUACAA | | TGAAAGTCCTTTCATGAATACAA | | AGGAAU-CGGAGA-6_1-2_bulge- |
| CCACGGCUAUUUGACGGAGAUUA | | CCACGGCTATTTGACGGAGATTA | | asymmetric_U-UU |
| CACCACACUGUCGUCGAAUGGCC | | CACCACACTGTCGTCGAATGGCC | | 0_1-1_mismatch_A-C |
| ACUCCCAGU | | ACTCCCAGT | | 4 1-1_mismatch_A-A |
| | | | | 28_6-6_internal_loop-symmetric_ |
| | | | | AGGCCA-ACAAAC |
| GCCACAACUCCCUCCUACAAACU | 347 | GCCACAACTCCCTCCTACAAACT | 364 | -10_6-6_internal_loop-symmetric_ |
| UGAAAGUCCUUUCAUGAAUACAA | | TGAAAGTCCTTTCATGAATACAA | | AGGAAU-CGGAGA-5_2-1_bulge- |
| CCACGGCUUUGACGGAGAUUACA | | CCACGGCTTTGACGGAGATTACA | | asymmetric_UU-U |
| CCACACUGUCGUCGAAUGGCCAC | | CCACACTGTCGTCGAATGGCCAC | | 0_1-1_mismatch_A-C |
| UCCCAGU | | TCCCAGT | | 4_1-1_mismatch_A-A |
| | | | | 28_6-6_internal_loop-symmetric_ |
| | | | | AGGCCA-ACAAAC |
| GCCACAACUCCCUCCUAUAGAGU | 348 | GCCACAACTCCCTCCTATAGAGT | 365 | -6_8-7_internal_loop-asymmetric_ |
| UGAAAGUCCUUUCAUGAAUACAU | | TGAAAGTCCTTTCATGAATACAT | | GAAUUCAU-UCGCCGA |
| CUACGGCUAUCGCCGACUUUACA | | CTACGGCTATCGCCGACTTTACA | | 0_1-1_mismatch_A-C |
| CCACACUGUCGUCGAAUGGCCAC | | CCACACTGTCGTCGAATGGCCAC | | 2 1-1_wobble G-U |
| UCCCAGU | | TCCCAGT | | 28_6-6_internal_loop-symmetric_ |
| | | | | AGGCCA-AUAGAG |
| GCCACAACUCCCUCCUAUAGAGU | 349 | GCCACAACTCCCTCCTATAGAGT | 366 | -5_9-8_internal_loop-asymmetric_ |
| UGAAAGUCCUUUCAUGAAUACAU | | TGAAAGTCCTTTCATGAATACAT | | GAAUUCAUU-CUCGCCGA |
| CUACGGCUCUCGCCGACUUUACA | | CTACGGCTCTCGCCGACTTTACA | | 0_1-1_mismatch_A-C |
| CCACACUGUCGUCGAAUGGCCAC | | CCACACTGTCGTCGAATGGCCAC | | 2_1-1_wobble_G-U |
| UCCCAGU | | TCCCAGT | | 28_6-6_internal_loop-symmetric_ |
| | | | | AGGCCA-AUAGAG |
| GCCACAACUCCCUCCUAUAGCGU | 350 | GCCACAACTCCCTCCTATAGCGT | 367 | -5_9-8_internal_loop-asymmetric_ |
| UGAAAGUCCUUUCAUGAAUACAU | | TGAAAGTCCTTTCATGAATACAT | | GAAUUCAUU-CUCGCCGA |
| CUACGGCUCUCGCCGACUUUACA | | CTACGGCTCTCGCCGACTTTACA | | 0_1-1_mismatch_A-C |
| CCACACUGUCGUCGAAUGGCCAC | | CCACACTGTCGTCGAATGGCCAC | | 2_1-1_wobble G-U |
| UCCCAGU | | TCCCAGT | | 28_2-1_bulge-asymmetric_AG-G |
| | | | | 32_2-3_bulge-asymmetric_CA-AUA |
| GCCACAACUCCCUCCUUGACGGC | 351 | GCCACAACTCCCTCCTTGACGGC | 368 | -16_6-6_internal_loop-symmetric_ |
| GGAAAGUCCUUUCAUGGCACAUC | | GGAAAGTCCTTTCATGGCACATC | | GUGUAA-ACCGUG |
| CACGGCUACCAAUUCCUACCGUG | | CACGGCTACCAATTCCTACCGTG | | -6_3-2_bulge-asymmetric_CAU-CC |
| CACACUGUCGUCGAAUGGCCACU | | CACACTGTCGTCGAATGGCCACT | | 0_1-1_mismatch_A-C |
| CCCAGU | | CCCAGT | | 8_2-1_bulge-asymmetric_AU-C |
| | | | | 10_1-1_wobble U-G |
| | | | | 26_4-4_bulge-symmetric_AAAG-GGCG |
| | | | | 31_1-1_mismatch_C-A |
| GCCACAACUCCCUCCUUGAUGGC | 352 | GCCACAACTCCCTCCTTGATGGC | 369 | -16_6-6_internal_loop-symmetric_ |
| GGAAAGUCCUUUCAUGGCACAUC | | GGAAAGTCCTTTCATGGCACATC | | GUGUAA-ACCGUG |
| CACGGCUACCAAUUCCUACCGUG | | CACGGCTACCAATTCCTACCGTG | | -6_3-2_bulge-asymmetric_CAU-CC |
| CACACUGUCGUCGAAUGGCCACU | | CACACTGTCGTCGAATGGCCACT | | 0_1-1_mismatch_A-C |
| CCCAGU | | CCCAGT | | 8_2-1_bulge-asymmetric_AU-C |
| | | | | 10_1-1_wobble U-G |
| | | | | 26_6-6_internal_loop-symmetric_ |
| | | | | AAAGGC-AUGGCG |
| GCCACAACUCCCUCCUUGAUGGC | 353 | GCCACAACTCCCTCCTTGATGGC | 370 | -16_6-6_internal_loop-symmetric_ |
| GGAAAGUCCUUUCAUGGCACAUC | | GGAAAGTCCTTTCATGGCACATC | | GUGUAA-ACCGUG |
| CACGGCUAUCAAUUCCUACCGUG | | CACGGCTATCAATTCCTACCGTG | | -6_3-2_bulge-asymmetric_CAU-UC |
| CACACUGUCGUCGAAUGGCCACU | | CACACTGTCGTCGAATGGCCACT | | 0_1-1_mismatch_A-C |
| CCCAGU | | CCCAGT | | 8_2-1_bulge-asymmetric_AU-C |
| | | | | 10_1-1_wobble_U-G |
| | | | | 26_6-6_internal_loop-symmetric_ |
| | | | | AAAGGC-AUGGCG |
| GCCACAACUCCCUCCUUGUAGGA | 354 | GCCACAACTCCCTCCTTGTAGGA | 371 | -16_6-6_internal_loop-symmetric_ |
| GGAAAGUCCUUUCAUUAAUACAU | | GGAAAGTCCTTTCATTAATACAT | | GUGUAA-CCUCUA |
| CCACGGCAAUGGAAUUCCUCCUC | | CCACGGCAATGGAATTCCTCCTC | | -6_2-2_bulge-symmetric_AU-UG |
| UACACACUGUCGUCGAAUGGCCA | | TACACACTGTCGTCGAATGGCCA | | -4_1-1_mismatch_A-A |
| CUCCCAGU | | CTCCCAGT | | 0_1-1_mismatch_A-C |
| | | | | 11 1-1_mismatch_C-U |
| | | | | 26_6-6_internal_loop-symmetric_ |
| | | | | AAAGGC-UAGGAG |
| GCCACAACUCCCUCCUUGUAGGA | 355 | GCCACAACTCCCTCCTTGTAGGA | 372 | -16_6-6_internal_loop-symmetric_ |
| GGAAAGUCCUUUCAUUAAUACAU | | GGAAAGTCCTTTCATTAATACAT | | GUGUAA-CCUCUA |
| CCACGGCACUUGGAAUUCCUCCU | | CCACGGCACTTGGAATTCCTCCT | | -4_4-5_internal_loop-asymmetric_ |
| CUACACACUGUCGUCGAAUGGCC | | CTACACACTGTCGTCGAATGGCC | | AUUA-ACUUG |
| ACUCCCAGU | | ACTCCCAGT | | 0_1-1_mismatch_A-C |
| | | | | 11_1-1_mismatch_C-U |
| | | | | 26_6-6_internal_loop-symmetric_ |
| | | | | AAAGGC-UAGGAG |
| GCCACAACUCCCUCCUUGUAGGA | 356 | GCCACAACTCCCTCCTTGTAGGA | 373 | -16 6-6_internal_loop-symmetric_ |
| GGAAAGUCCUUUCAUUAAUACAU | | GGAAAGTCCTTTCATTAATACAT | | GUGUAA-CCUCUA |
| CCACGGCUAUGGAAUUCCUCCUC | | CCACGGCTATGGAATTCCTCCTC | | -6_2-2_bulge-symmetric_AU-UG |
| UACACACUGUCGUCGAAUGGCCA | | TACACACTGTCGTCGAATGGCCA | | 0_1-1_mismatch_A-C |
| CUCCCAGU | | CTCCCAGT | | 11_1-1_mismatch_C-U |
| | | | | 26_6-6_internal_loop-symmetric_ |
| | | | | AAAGGC-UAGGAG |
| GCCACAACUCCCUCCUUGUAGGA | 357 | GCCACAACTCCCTCCTTGTAGGA | 374 | -16_6-6_internal_loop-symmetric_ |
| GGAAAGUCCUUUCAUUAAUACAU | | GGAAAGTCCTTTCATTAATACAT | | GUGUAA-CCUCUA |
| CCACGGCUCUUGGAAUUCCUCCU | | CCACGGCTCTTGGAATTCCTCCT | | -5_3-4_bulge-asymmetric_AUU-CUUG |
| CUACACACUGUCGUCGAAUGGCC | | CTACACACTGTCGTCGAATGGCC | | 0_1-1_mismatch_A-C |
| ACUCCCAGU | | ACTCCCAGT | | 11_1-1_mismatch_C-U |
| | | | | 26_6-6_internal_loop-symmetric_ |
| | | | | AAAGGC-UAGGAG |

### Example 10

### SNCA Guide Design And Hybridization In Non-Human Primates

In-vivo molecular efficiency is evaluated by treating NHP subjects with an AAV vector comprising a sequence encoding a guide RNA of SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 334, or SEQ ID NO: 340 that targets the SNCA TIS. RNA editing and protein expression of SNCA is evaluated. Readouts also include SNCA target site editing by RNA sequences, gRNA quantification in tissues, and identification of splice variants of SNCA by sequencing analysis. These results are compared with assays performed in human iPSCs.

Biodistribution assays are performed in vivo. The biodistribution of the vector comprising the guide RNA sequence is also assessed by measuring the viral genomes per diploid genome and gRNA expression in NHP tissues. For example, gRNA expression and SNCA expression is performed in brain, liver, and kidney. Urine is collected from treated animals to determine excretion rates of the gRNA and determine the appropriate AAV-SNCA gRNA dose.

In order to determine the safety and efficacy of SNCA-targeting gRNAs, off-target RNA editing assessment of NHP tissues is performed as well as monitoring NHPs for markers of neuronal damage, using biomarkers such as Neurofilament Light (NfL) in the cerebral spinal fluid (CSF) and serum. NfL and SNCA proteins are measured via an ELISA assay comparing control and gRNA-treated NHP biological samples. During axonal damage in the CNS, NfL leaks into CSF and can often also be detected in serum and plasma. NfL levels are used to determine the effect of SNCA-targeting gRNAs on NHP neurons by establishing a baseline NfL prior to administration of gRNAs and comparing treated animals with untreated animals. NfL is used to identify the presence of any adverse reactions to treatment in the brain. Biological samples from cynomolgus NHPs are also evaluated by histopathology and toxicology assays to monitor changes in disease pathology.

### Example 11

### Treatment of a Synucleinopathy in a Human Subject

A human subject having a synucleinopathy is administered an AAV vector comprising a sequence encoding a guide RNA of SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 334, or SEQ ID NO: 340 that targets the SNCA TIS. The subject is administered a therapeutic dose of the AAV-virus and is monitored for improvements in symptoms associated with the synucleinopathy. Each of the AAV-delivered SNCA TIS-targeting guide RNAs when administered to the subject improves one or more symptoms of the synucleinopathy in the human subject.

### Example 12

### Treatment of Parkinson's Disease in a Human Subject

A human subject diagnosed with Parkinson's disease is administered an AAV vector comprising a sequence encoding a guide RNA of SEQ ID NO: 293, SEQ ID NO: 294, SEQ ID NO: 295, SEQ ID NO: 296, SEQ ID NO: 297, SEQ ID NO: 298, SEQ ID NO: 299, SEQ ID NO: 334, or SEQ ID NO: 340 that targets the SNCA TIS. The subject is administered a therapeutic dose of the AAV-virus and is monitored for improvements in behavioral and cognitive ability. Patients that are administered the AAV-gRNAs that target the SNCA TIS show an increase Unified Parkinson's Disease Rating Scale (UPDRS) scores, relative to prior to receiving treatment, thereby treating Parkinson's disease in the human subject.

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. An engineered guide RNA, or an engineered polynucleotide encoding the engineered guide RNA, wherein the engineered guide RNA comprises from 85 to 90 bases and has at least 80% sequence identity to any one of SEQ ID NO: 294, SEQ ID NO: 296, or SEQ ID NO: 297.
Embodiment 2. An engineered guide RNA, or an engineered polynucleotide encoding the engineered guide RNA, wherein the engineered guide RNA comprises from 95 to 100 bases and has at least 80% sequence identity to any one of SEQ ID NO: 293, SEQ ID NO: 299, SEQ ID NO: 334, or SEQ ID NO: 340.
Embodiment 3. An engineered guide RNA, or an engineered polynucleotide encoding the engineered guide RNA, wherein the engineered guide RNA has at least 80% sequence identity to any one of SEQ ID NO: 296, SEQ ID NO: 299, SEQ ID NO: 334, or SEQ ID NO: 340, wherein, upon hybridization of the engineered guide RNA to a target sequence of a target SNCA RNA, a guide-target RNA scaffold is formed that comprises a 6/6 symmetric internal loop; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii); and wherein the 6/6 symmetric internal loop is at position −6, relative to the target adenosine at position 0.
Embodiment 4. An engineered guide RNA, or an engineered polynucleotide encoding the engineered guide RNA, wherein the engineered guide RNA has at least 80% sequence identity to SEQ ID NO: 293, wherein, upon hybridization of the engineered guide RNA to a target sequence of a target SNCA RNA, a guide-target RNA scaffold is formed that comprises a 6/6 symmetric internal loop; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii); and wherein the 6/6 symmetric internal loop is at position −8, relative to the target adenosine at position 0.
Embodiment 5. An engineered guide RNA, or an engineered polynucleotide encoding the engineered guide RNA, wherein the engineered guide RNA has at least 80% sequence identity to SEQ ID NO: 294, wherein, upon hybridization of the engineered guide RNA to a target sequence of a target SNCA RNA, a guide-target RNA scaffold is formed that comprises a 6/6 symmetric internal loop; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii); and wherein the 6/6 symmetric internal loop is at position −10, relative to the target adenosine at position 0.
Embodiment 6. An engineered guide RNA or an engineered polynucleotide encoding the engineered guide RNA, wherein the engineered guide RNA has at least 80% sequence identity to SEQ ID NO: 297, wherein, upon hybridization of the engineered guide RNA to a target sequence of a target SNCA RNA, a guide-target RNA scaffold is formed that comprises a 6/6 symmetric internal loop; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii); and wherein the 6/6 symmetric internal loop is at position −10, relative to the target adenosine at position 0.
Embodiment 7. An engineered guide RNA or an engineered polynucleotide encoding the engineered guide RNA, wherein the engineered guide RNA comprises a polynucleotide sequence having at least about: 80%, 85%, 90%, 92%, 95%, 97%, or 99% sequence identity to any one of SEQ ID NOS: 293-299, wherein, upon hybridization of the engineered guide RNA to a target sequence of a target SNCA RNA, a guide-target RNA scaffold is formed that has one or more structural features selected from the group consisting of: a bulge, an internal loop, a wobble base pair, or a hairpin; wherein the structural feature is not present within the engineered guide RNA prior to the hybridization of the engineered guide RNA to the target sequence of the target SNCA RNA; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii).
Embodiment 8. An engineered guide RNA or an engineered polynucleotide encoding the engineered guide RNA, wherein the engineered guide RNA comprises a polynucleotide sequence having at least about: 80%, 85%, 90%, 92%, 95%, 97%, or 99% sequence identity to any one of SEQ ID NOS: 334-340, wherein, upon hybridization of the engineered guide RNA to a target sequence of a target SNCA RNA, a guide-target RNA scaffold is formed that has one or more structural features selected from the group consisting of: a bulge, an internal loop, a wobble base pair, or a hairpin; wherein the structural feature is not present within the engineered guide RNA prior to the hybridization of the engineered guide RNA to the target sequence of the target SNCA RNA; wherein upon hybridization of the engineered guide RNA to the target sequence of the target SNCA RNA: (i) the engineered guide RNA facilitates RNA editing of a target adenosine in the target sequence of the target SNCA RNA by an RNA editing entity; (ii) the engineered guide RNA facilitates exon skipping of an exon of the target SNCA RNA that comprises the target adenosine; or (iii) both (i) and (ii).
Embodiment 9. An engineered guide RNA or an engineered polynucleotide encoding the engineered guide RNA, wherein the engineered guide RNA comprises a polynucleotide sequence having at least about: 80%, 85%, 90%, 92%, 95%, 97%, or 99% sequence identity to any one of SEQ ID NO: 293, SEQ ID NO: 298, or SEQ ID NO: 334-SEQ ID NO: 340, wherein, upon hybridization of the engineered guide RNA to a target sequence of a target SNCA RNA, a guide-target RNA scaffold is formed that has one or more wobble base pairs.
Embodiment 10. A composition that comprises the engineered guide RNA, or the engineered polynucleotide encoding the engineered guide RNA, of any one of Embodiment 1-Embodiment 9.
Embodiment 11. A composition comprising an engineered guide RNA or a polynucleotide encoding the engineered guide RNA, wherein the engineered guide RNA has complementarity to a target sequence of a target SNCA RNA and comprises a polynucleotide sequence having at least about: 80%, 85%, 90%, 92%, 95%, 97%, or 99% sequence identity to any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784; wherein upon hybridization of the engineered guide RNA to the target sequence of the target SNCA RNA, a guide-target RNA scaffold is formed that has one or more structural features that is not present within the engineered guide RNA prior to the hybridization and is selected from the group consisting of: a bulge, an internal loop, and a hairpin; and wherein formation of the guide-target RNA scaffold results in knockdown of alpha-synuclein protein encoded by the target SNCA RNA.
Embodiment 12. The composition of Embodiment 11, wherein the engineered guide RNA comprises a polynucleotide sequence having at least about: 80%, 85%, 90%, 92%, 95%, 97%, or 99% sequence identity to any one of SEQ ID NOS: 293-299 or SEQ ID NOS: 333-340.
Embodiment 13. The composition of Embodiment 12, wherein the engineered guide RNA comprises the polynucleotide sequence of any one of SEQ ID NOS: 293-299 or SEQ ID NOS: 333-340.
Embodiment 14. The composition of Embodiment 11, wherein the target sequence of the target SNCA RNA comprises a translation initiation site.
Embodiment 15. The composition of Embodiment 14, wherein the translation initiation site is the SNCA Codon 1 translation initiation site of Exon 2.
Embodiment 16. The composition of Embodiment 15, wherein the translation initiation site is the SNCA Codon 1 translation initiation site of Exon 2 corresponding to position 226 of an SNCA transcript variant 1 of accession number NM_000345.4.
Embodiment 17. The composition of Embodiment 11, wherein the one or more structural features comprises at least a first 6/6 symmetric internal loop and at least a second 6/6 symmetric loop.
Embodiment 18. The composition of Embodiment 17, wherein the first 6/6 symmetric internal loop is at a position selected from the group consisting of: 33, 32, 30, 28, and 26; relative to a target adenosine at position 0.
Embodiment 19. The composition of Embodiment 18, wherein the first 6/6 symmetric internal loop is at position 33, relative to the target adenosine at position 0.
Embodiment 20. The composition of Embodiment 19, wherein the one or more structural features further comprises at least one structural feature selected from the group consisting of: a G/U wobble base at position −40 relative to position 0, a U/G wobble base at position −36 relative to position 0, a G/U wobble base at position −30 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof.
Embodiment 21. The composition of Embodiment 20, wherein the engineered guide RNA comprises at least about: 80%, 85%, 90%, 92%, 95%, 97%, or 99% sequence identity to SEQ ID NO: 337.
Embodiment 22. The composition of Embodiment 19, wherein the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, the second 6/6 symmetric internal loop at position −6 relative to position 0, an A/C mismatch at position 0, a G/U wobble base at position 2 relative to position 0, a 2/2 symmetric bulge at position 33 relative to position 0, a G/G mismatch at position 36 relative to position 0, a G/G mismatch at position 38 relative to position 0, and any combination thereof.
Embodiment 23. The composition of Embodiment 22, wherein the engineered guide RNA comprises at least about: 80%, 85%, 90%, 92%, 95%, 97%, or 99% sequence identity to SEQ ID NO: 338 or SEQ ID NO: 339.
Embodiment 24. The composition of Embodiment 19, wherein the one or more structural features further comprises at least one structural feature selected from the group consisting of: a G/U wobble base at position −40 relative to position 0, a U/G wobble base at position −36 relative to position 0, a G/U wobble base at position −30 relative to position 0, a U/G wobble base at position −25 relative to position 0, a U/G wobble base at position −23 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, an A/C mismatch at position 0, a G/U wobble base at position 2 relative to position 0, a U/G wobble base at position 5 relative to position 0, a 2/2 symmetric bulge at position 33 relative to position 0, a G/G mismatch at position 36 relative to position 0, a G/G mismatch at position 38 relative to position 0, and any combination thereof.
Embodiment 25. The composition of Embodiment 24, wherein the engineered guide RNA comprises at least about: 80%, 85%, 90%, 92%, 95%, 97%, or 99% sequence identity to SEQ ID NO: 340.
Embodiment 26. The composition of Embodiment 19, wherein the one or more structural features further comprises at least one structural feature selected from the group consisting of: the second 6/6 symmetric internal loop at position −6 relative to position 0, an A/C mismatch at position 0, and any combination thereof.
Embodiment 27. The composition of Embodiment 26, wherein the engineered guide RNA comprises at least about: 80%, 85%, 90%, 92%, 95%, 97%, or 99% sequence identity to SEQ ID NO: 298 or SEQ ID NO: 299.
Embodiment 28. The composition of Embodiment 19, wherein the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof.
Embodiment 29. The composition of Embodiment 28, wherein the engineered guide RNA comprises at least about: 80%, 85%, 90%, 92%, 95%, 97%, or 99% sequence identity to SEQ ID NO: 299.
Embodiment 30. The composition of Embodiment 18, wherein the first 6/6 symmetric internal loop is at position 32, relative to the target adenosine at position 0.
Embodiment 31. The composition of Embodiment 30, wherein the one or more structural features further comprises at least one structural feature selected from the group consisting of: the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof.
Embodiment 32. The composition of Embodiment 31, wherein the engineered guide RNA comprises at least about: 80%, 85%, 90%, 92%, 95%, 97%, or 99% sequence identity to SEQ ID NO: 333.
Embodiment 33. The composition of Embodiment 30, wherein the one or more structural features further comprises at least one structural feature selected from the group consisting of: a G/U wobble base at position −40 relative to position 0, a U/G wobble base at position −36 relative to position 0, a G/U wobble base at position −33 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof.
Embodiment 34. The composition of Embodiment 33, wherein the engineered guide RNA comprises at least about: 80%, 85%, 90%, 92%, 95%, 97%, or 99% sequence identity to SEQ ID NO: 334.
Embodiment 35. The composition of Embodiment 30, wherein the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, the second 6/6 symmetric internal loop at position −6 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof.
Embodiment 36. The composition of Embodiment 35, wherein the engineered guide RNA comprises at least about: 80%, 85%, 90%, 92%, 95%, 97%, or 99% sequence identity to SEQ ID NO: 335.
Embodiment 37. The composition of Embodiment 30, wherein the one or more structural features further comprises at least one structural feature selected from the group consisting of: a G/U wobble base at position −40 relative to position 0, a U/G wobble base at position −36 relative to position 0, a G/U wobble base at position −33 relative to position 0, a G/U wobble base at position −30 relative to position 0, a U/G wobble base at position −25 relative to position 0, a G/U wobble base at position −21 relative to position 0, a U/G wobble base at position −18 relative to position 0, a G/U wobble base at position −13 relative to position 0, the second 6/6 symmetric internal loop at position −6 relative to position 0, a G/U wobble base at position −3 relative to position 0, an A/C mismatch at position 0, a 3/3 symmetric bulge at position 13 relative to position 0, a 0/1 asymmetric bulge at position 31 relative to position 0, a 5/4 asymmetric internal loop at position 33 relative to position 0, a C/A mismatch at position 55 relative to position 0, and any combination thereof.
Embodiment 38. The composition of Embodiment 37, wherein the engineered guide RNA comprises at least about: 80%, 85%, 90%, 92%, 95%, 97%, or 99% sequence identity to SEQ ID NO: 336.
Embodiment 39. The composition of Embodiment 30, wherein the one or more structural features further comprises at least one structural feature selected from the group consisting of: the second 6/6 symmetric internal loop at position −6 relative to position 0, an A/C mismatch at position 0, and any combination thereof.
Embodiment 40. The composition of Embodiment 39, wherein the engineered guide RNA comprises at least about: 80%, 85%, 90%, 92%, 95%, 97%, or 99% sequence identity to SEQ ID NO: 295.
Embodiment 41. The composition of Embodiment 18, wherein the first 6/6 symmetric internal loop is at position 30, relative to the target adenosine at position 0.
Embodiment 42. The composition of Embodiment 41, wherein the one or more structural features further comprises at least one structural feature selected from the group consisting of: the second 6/6 symmetric internal loop at position −18 relative to position 0, a 3/3 symmetric bulge at position −6 relative to position 0, an A/C mismatch at position 0, a G/G mismatch at position 6 relative to position 0, a U/C mismatch at position 10 relative to position 0, and any combination thereof.
Embodiment 43. The composition of Embodiment 42, wherein the engineered guide RNA comprises at least about: 80%, 85%, 90%, 92%, 95%, 97%, or 99% sequence identity to SEQ ID NO: 297.
Embodiment 44. The composition of Embodiment 18, wherein the first 6/6 symmetric internal loop is at position 28, relative to the target adenosine at position 0.
Embodiment 45. The composition of Embodiment 44, wherein the one or more structural features further comprises at least one structural feature selected from the group consisting of: the second 6/6 symmetric internal loop at position −8 relative to position 0, an A/C mismatch at position 0, a G/U wobble base pair at position 2 relative to position 0, and any combination thereof.
Embodiment 46. The composition of Embodiment 45, wherein the engineered guide RNA comprises at least about: 80%, 85%, 90%, 92%, 95%, 97%, or 99% sequence identity to SEQ ID NO: 293.
Embodiment 47. The composition of Embodiment 44, wherein the one or more structural features further comprises at least one structural feature selected from the group consisting of: the second 6/6 symmetric internal loop at position −10 relative to position 0, a 0/1 asymmetric bulge at position −6 relative to position 0, an A/C mismatch at position 0, an A/A mismatch at position 4 relative to position 0, and any combination thereof.
Embodiment 48. The composition of Embodiment 47, wherein the engineered guide RNA comprises at least about: 80%, 85%, 90%, 92%, 95%, 97%, or 99% sequence identity to SEQ ID NO: 294.
Embodiment 49. The composition of Embodiment 18, wherein the first 6/6 symmetric internal loop is at position 26, relative to the target adenosine at position 0.
Embodiment 50. The composition of Embodiment 49, wherein the one or more structural features further comprises at least one structural feature selected from the group consisting of: the second 6/6 symmetric internal loop at position −6 relative to position 0, an A/C mismatch at position 0, a 2/2 symmetric bulge at position 5 relative to position 0, and any combination thereof.
Embodiment 51. The composition of Embodiment 50, wherein the engineered guide RNA comprises at least about: 80%, 85%, 90%, 92%, 95%, 97%, or 99% sequence identity to SEQ ID NO: 296.
Embodiment 52. The composition of Embodiment 11, wherein the one or more structural features comprises the bulge, and wherein the bulge is a symmetric bulge.
Embodiment 53. The composition of Embodiment 11, wherein the one or more structural features comprises the bulge, and wherein the bulge is an asymmetric bulge.
Embodiment 54. The composition of Embodiment 11, wherein the one or more structural features comprises the internal loop, and wherein the internal loop is a symmetric internal loop.
Embodiment 55. The composition of Embodiment 11, wherein the one or more structural features comprises the internal loop, and wherein the internal loop is an asymmetric internal loop.
Embodiment 56. The composition of Embodiment 11, wherein the guide-target RNA scaffold comprises a wobble base pair.
Embodiment 57. The composition of Embodiment 11, wherein the one or more structural features comprises the hairpin, and wherein the hairpin is a recruitment hairpin or a non-recruitment hairpin.
Embodiment 58. The composition of any one of Embodiment 11-Embodiment 57, wherein upon hybridization of the engineered guide RNA to the target sequence of the target SNCA RNA, the engineered guide RNA facilitates RNA editing of one or more adenosines in the target sequence of the target SNCA RNA by an RNA editing entity.
Embodiment 59. The composition of Embodiment 58, wherein the RNA editing entity comprises ADAR1, ADAR2, ADAR3, or any combination thereof.
Embodiment 60. The composition of any one of Embodiment 11-Embodiment 59, wherein the composition comprises the engineered polynucleotide encoding the engineered guide RNA.
Embodiment 61. The composition of Embodiment 60, wherein the engineered polynucleotide is comprised in or on a vector.
Embodiment 62. The composition of Embodiment 61, wherein the vector is a viral vector, and wherein the engineered polynucleotide is encapsidated in the viral vector.
Embodiment 63. The composition of Embodiment 61, wherein the viral vector is an adeno-associated viral (AAV) vector, a derivative thereof.
Embodiment 64. The composition of Embodiment 63, wherein the viral vector is an adeno-associated viral (AAV) and wherein the AAV vector is an AAV1 vector, AAV2 vector, AAV3 vector, AAV4 vector, AAV5 vector, AAV6 vector, AAV7 vector, AAV8 vector, AAV9 vector, AAV10 vector, AAV 11 vector, or a derivative, a chimera, or a variant of any of these.
Embodiment 65. The composition of any one of Embodiment 63-Embodiment 64, wherein the AAV vector is a recombinant AAV (rAAV) vector, a hybrid AAV vector, a chimeric AAV vector, a self-complementary AAV (scAAV) vector, or any combination thereof.
Embodiment 66. The composition of Embodiment 11, wherein the target sequence of the target SNCA RNA has a polynucleotide sequence that is at least 80% identical to SEQ ID NO: 300.
Embodiment 67. The composition of any one of Embodiment 52-Embodiment 66, wherein the engineered guide RNA comprises the sequence of any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784.
Embodiment 68. A composition that comprises an engineered guide RNA or a polynucleotide encoding the engineered guide RNA, wherein the engineered guide RNA has a length of from 85 nucleotides to 100 nucleotides and hybridizes to at least 80 bases of a target RNA sequence, and the target RNA sequence has at least 80%, at least 85%, at least 90%, at least 92%, at least 95%, at least 96%, at least 97%, at least 98%, at least 99%, or 100% sequence identity to

(SEQ ID NO: 300)
GCCAUUCGACGACAGUGUGGUGUAAAGGAAUUCAUUAGCCAUGGAUGUAU
UCAUGAAAGGACUUUCAAAGGCCAAGGAGGGAGUUGUGGCUGCUGCUGA
G.

Embodiment 69. The composition of Embodiment 68, wherein upon hybridization of the engineered guide RNA to the at least 80 bases of the target RNA sequence, the engineered guide RNA facilitates RNA editing of one or more adenosines in the at least 80 bases of the target RNA sequence by an RNA editing entity.
Embodiment 70. The composition of Embodiment 69, wherein the engineered guide RNA, upon hybridization to the at least 80 bases of the target RNA sequence, forms a guide-target RNA scaffold that comprises one or more structural features.
Embodiment 71. The composition of Embodiment 70, wherein the one or more structural features comprises a bulge, wherein the bulge is a symmetric bulge.
Embodiment 72. The composition of Embodiment 70, wherein the one or more structural features comprises a bulge, wherein the bulge is an asymmetric bulge.
Embodiment 73. The composition of Embodiment 70, wherein the one or more structural features comprises an internal loop, wherein the internal loop is a symmetric internal loop.
Embodiment 74. The composition of Embodiment 70, wherein the one or more structural features comprises an internal loop, wherein the internal loop is an asymmetric internal loop.
Embodiment 75. The composition of Embodiment 70, wherein the one or more structural features comprises a Wobble base pair.
Embodiment 76. The composition of Embodiment 70, wherein the one or more structural features comprises a hairpin, wherein the hairpin is a recruitment hairpin or a non-recruitment hairpin.
Embodiment 77. The composition of any one of Embodiment 69-Embodiment 76, wherein the RNA editing entity comprises ADAR1, ADAR2, ADAR3, or any combination thereof.
Embodiment 78. The composition of Embodiment 68, wherein the composition comprises an engineered polynucleotide encoding the engineered guide RNA.
Embodiment 79. The composition of claim Embodiment 78, wherein the engineered polynucleotide encoding the engineered guide RNA is comprised in or on a vector.
Embodiment 80. The composition of Embodiment 79, wherein the vector is a viral vector, and wherein the engineered polynucleotide encoding the engineered guide RNA is encapsidated in the viral vector.
Embodiment 81. The composition of Embodiment 80, wherein the viral vector is an adeno-associated viral (AAV) vector or a derivative thereof.
Embodiment 82. The composition of Embodiment 81, wherein the AAV vector is an AAV1 vector, AAV2 vector, AAV3 vector, AAV4 vector, AAV5 vector, AAV6 vector, AAV7 vector, AAV8 vector, AAV9 vector, AAV10 vector, AAV11 vector, or a derivative, a chimera, or a variant thereof.
Embodiment 83. The composition of Embodiment 81 or Embodiment 82, wherein the AAV vector is a recombinant AAV (rAAV) vector, a hybrid AAV vector, a chimeric AAV vector, a self-complementary AAV (scAAV) vector, or any combination thereof.
Embodiment 84. A method of treating a disease or a condition in a subject in need thereof, the method comprising administering to the subject a therapeutically effective amount of the engineered guide RNA, or the engineered polynucleotide encoding the engineered guide RNA, of any one of Embodiment 1-Embodiment 9, or the composition of any one of Embodiment 10-Embodiment 83.
Embodiment 85. The method of Embodiment 84, wherein the disease or condition comprises a synucleinopathy.
Embodiment 86. The method of Embodiment 85, wherein the synucleinopathy comprises a Parkinson's disease.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

1. A composition comprising an engineered guide RNA or a polynucleotide encoding the engineered guide RNA, wherein the engineered guide RNA has complementarity to a target sequence of a target SNCA RNA and comprises a polynucleotide sequence having at least 80% sequence identity to any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784.

2. (canceled)

3. (canceled)

4. The composition of claim 1, wherein the target sequence of the target SNCA RNA comprises a translation initiation site.

5. The composition of claim 4, wherein the translation initiation site is the SNCA Codon 1 translation initiation site of Exon 2.

6. The composition of claim 5, wherein the translation initiation site is the SNCA Codon 1 translation initiation site of Exon 2 corresponding to position 226 of an SNCA transcript variant 1 of accession number NM_000345.4.

7. The composition of claim 1, wherein upon hybridization of the engineered guide RNA to the target sequence of the target SNCA RNA, a guide-target RNA scaffold is formed that has one or more structural features that is not present within the engineered guide RNA prior to the hybridization, and wherein the one or more structural features comprises at least a first 6/6 symmetric internal loop and at least a second 6/6 symmetric loop.

8. The composition of claim 7, wherein the first 6/6 symmetric internal loop is at a position selected from the group consisting of: 33, 32, 30, 28, and 26; relative to a target adenosine at position 0.

9. The composition of claim 8, wherein the first 6/6 symmetric internal loop is at position 33, relative to the target adenosine at position 0.

10. (canceled)

11. The composition of claim 9, wherein the engineered guide RNA comprises at least 80% sequence identity to an one of SEQ ID NO: 337, SEQ ID NO: 338, SEQ ID NO: 339, SEQ ID NO: 340, SEQ ID NO: 298, SEQ ID NO: 299, or SEQ ID NO: 299.

12.-19. (canceled)

20. The composition of claim 8, wherein the first 6/6 symmetric internal loop is at position 32, relative to the target adenosine at position 0.

21. (canceled)

22. The composition of claim 20, wherein the engineered guide RNA comprises at least 80% sequence identity to an one of SEQ ID NO: 333, SEQ ID NO: 334, SEQ ID NO: 335, SEQ ID NO: 336, or SEQ ID NO: 295.

23.-30. (canceled)

31. The composition of claim 8, wherein the first 6/6 symmetric internal loop is at position 30, relative to the target adenosine at position 0.

32. (canceled)

33. The composition of claim 31, wherein the engineered guide RNA comprises at least 80% sequence identity to SEQ ID NO: 297.

34. The composition of claim 8, wherein the first 6/6 symmetric internal loop is at position 28, relative to the target adenosine at position 0.

35. (canceled)

36. The composition of claim 34, wherein the engineered guide RNA comprises at least 80% sequence identity to SEQ ID NO: 293 or SEQ ID NO: 294.

37. (canceled)

38. (canceled)

39. The composition of claim 8, wherein the first 6/6 symmetric internal loop is at position 26, relative to the target adenosine at position 0.

40. (canceled)

41. The composition of claim 39, wherein the engineered guide RNA comprises at least 80% sequence identity to SEQ ID NO: 296.

42.-47. (canceled)

48. The composition of claim 1, wherein upon hybridization of the engineered guide RNA to the target sequence of the target SNCA RNA, the engineered guide RNA facilitates RNA editing of one or more adenosines in the target sequence of the target SNCA RNA by an ADAR enzyme.

49. (canceled)

50. (canceled)

51. (canceled)

52. The composition of claim 51, wherein the engineered guide RNA is encoded by a viral vector.

53. The composition of claim 52, wherein the viral vector is an adeno-associated viral (AAV) vector.

54. The composition of claim 53, wherein the AAV vector is an AAV1 vector, AAV2 vector, AAV3 vector, AAV4 vector, AAV5 vector, AAV6 vector, AAV7 vector, AAV8 vector, AAV9 vector, AAV10 vector, AAV 11 vector, or a derivative, a chimera, or a variant of any of these.

55.-83. (canceled)

84. A method of treating a Parkinson's disease in a subject in need thereof, the method comprising: administering to the subject a composition that comprises an engineered guide RNA or a polynucleotide encoding the engineered guide RNA, wherein the engineered guide RNA has complementarity to a target sequence of a target SNCA RNA and comprises a polynucleotide sequence having at least 80% sequence identity to any one of SEQ ID NOS: 2-285, SEQ ID NOS: 293-299, SEQ ID NOS: 333-357, SEQ ID NOS: 375-376, SEQ ID NOS: 380-381, SEQ ID NOS: 390-440, or SEQ ID NOS: 777-784, wherein the administering results in treatment of the Parkinson's disease in the subject.

85. The method of claim 84, wherein the treating comprises reducing one or more symptoms of the Parkinson's disease in the subject selected from the group consisting of muscle tone rigidity, bradykinesia, resting tremor, and any combination thereof, relative to prior to the administering.

86.-117. (canceled)

118. The composition of claim 1, wherein hybridization of the engineered guide RNA to the target sequence of the target SNCA RNA results in knockdown of alpha-synuclein protein encoded by the target SNCA RNA.


SEQUENCE LISTING (813 sequences)

SEQ ID NO: 1 - RNA, 45 nt, unidentified
  source 1..45: mol_type = unassigned RNA
gtggaatagtataacaatatgctaaatgttgttatagtatcccac

SEQ ID NO: 2 - RNA, 101 nt, synthetic construct
  source 1..101: mol_type = other RNA
ctcagcagcagccacaactccctcgcgcaacttaggaaagtgggttcatgcatagatccacggctacataattccttttggagtcactgtcgtcgaatggc

SEQ ID NO: 3 - RNA, 99 nt, synthetic construct
  source 1..99: mol_type = other RNA
ctcagcagcagccacaactccctcgcgcaactgggaaaggctttcatgcatagatccacggctacataattccttttggagtcactgtcgtcgaatggc

SEQ ID NO: 4 - RNA, 98 nt, synthetic construct
  source 1..98: mol_type = other RNA
ctcagcagcagccacaactccctcgcgcaacttcaagtggtttcatgcatagatccacggctacataattccttttggagtcactgtcgtcgaatggc

SEQ ID NO: 5 - RNA, 100 nt, synthetic construct
  source 1..100: mol_type = other RNA
ctcagcagcagccacaactccctcgcgcaacgaagaacaacctttcatgcatagatccacggctacataattccttttggagtcactgtcgtcgaatggc

SEQ ID NO: 6 - RNA, 101 nt, synthetic construct
  source 1..101: mol_type = other RNA
ctcagcagcagccacaactccctcgcgcaacttccgaaaggactttcatgcatagatccacggctacataattccttttggagtcactgtcgtcgaatggc

SEQ ID NO: 7 - RNA, 101 nt, synthetic construct
  source 1..101: mol_type = other RNA
ctcagcagcagccacaactccctcgcgcaacgacgaaacatcctttcatgcatagatccacggctacataattccttttggagtcactgtcgtcgaatggc

SEQ ID NO: 8 - RNA, 101 nt, synthetic construct
  source 1..101: mol_type = other RNA
ctcagcagcagccacaactccctcgcgcaacgcggaaagcccctttcatgcatagatccacggctacataattccttttggagtcactgtcgtcgaatggc

SEQ ID NO: 9 - RNA, 100 nt, synthetic construct
  source 1..100: mol_type = other RNA
ctcagcagcagccacaactccctcgcgcaactgccaaagaactttcatgcatagatccacggctacataattccttttggagtcactgtcgtcgaatggc

SEQ ID NO: 10 - RNA, 103 nt, synthetic construct
  source 1..103: mol_type = other RNA
ctcagcagcagccacaactccctcgcgcaacttcggaaagcagcctttcatgcatagatccacggctacataattccttttggagtcactgtcgtcgaatggc

SEQ ID NO: 11 - RNA, 99 nt, synthetic construct
  source 1..99: mol_type = other RNA
ctcagcagcagccacaactccctcgcgcaacattgaaagtcgttcatgcatagatccacggctacataattccttttggagtcactgtcgtcgaatggc

SEQ ID NO: 12 - RNA, 100 nt, synthetic construct
  source 1..100: mol_type = other RNA
ctcagcagcagccacaactccctcgcgcaacttcgaaagtgacttcatgcatagatccacggctacataattccttttggagtcactgtcgtcgaatggc

SEQ ID NO: 13 - RNA, 98 nt, synthetic construct
  source 1..98: mol_type = other RNA
ctcagcagcagccacaactccctcgcgcaacgatgattcctttcatgcatagatccacggctacataattccttttggagtcactgtcgtcgaatggc

SEQ ID NO: 14 - RNA, 99 nt, synthetic construct
  source 1..99: mol_type = other RNA
ctcagcagcagccacaactccctcgcgcaactgcaaaagtccatcatgcatagatccacggctacataattccttttggagtcactgtcgtcgaatggc

SEQ ID NO: 15 - RNA, 101 nt, synthetic construct
  source 1..101: mol_type = other RNA
ctcagcagcagccacaactccctcgcgcaactttgaaccgtccttccatgcatagatccacggctacataattccttttggagtcactgtcgtcgaatggc

SEQ ID NO: 16 - RNA, 99 nt, synthetic construct
  source 1..99: mol_type = other RNA
ctcagcagcagccacaactccctcgcgcaactttgatgtgatttcatgcatagatccacggctacataattccttttggagtcactgtcgtcgaatggc

SEQ ID NO: 17 - RNA, 99 nt, synthetic construct
  source 1..99: mol_type = other RNA
ctcagcagcagccacaactccctcgcgcaactcctgaaagtgttcatgcatagatccacggctacataattccttttggagtcactgtcgtcgaatggc

SEQ ID NO: 18 - RNA, 100 nt, synthetic construct
  source 1..100: mol_type = other RNA
ctcagcagcagccacaactccctcgcgcaactttacaaaagtgttcatgcatagatccacggctacataattccttttggagtcactgtcgtcgaatggc

SEQ ID NO: 19 - RNA, 99 nt, synthetic construct
  source 1..99: mol_type = other RNA
ctcagcagcagccacaactccctcgcgcaactttgacgtagtttcatgcatagatccacggctacataattccttttggagtcactgtcgtcgaatggc

SEQ ID NO: 20 - RNA, 100 nt, synthetic construct
  source 1..100: mol_type = other RNA
ctcagcagcagccacaactccctcgcgcaacggtgaaagtcaattcatgcatagatccacggctacataattccttttggagtcactgtcgtcgaatggc

SEQ ID NO: 21 - RNA, 100 nt, synthetic construct
  source 1..100: mol_type = other RNA
ctcagcagcagccacaactccctcgcgcaactgcgacgctcctttcatgcatagatccacggctacataattccttttggagtcactgtcgtcgaatggc

SEQ ID NO: 22 - RNA, 99 nt, synthetic construct
  source 1..99: mol_type = other RNA
ctcagcagcagccacaactccctcgcgcaactttgctaagtcatcatgcatagatccacggctacataattccttttggagtcactgtcgtcgaatggc

SEQ ID NO: 23 - RNA, 100 nt, synthetic construct
  source 1..100: mol_type = other RNA
ctcagcagcagccacaactccctcgcgcaactgtgaaagtgatttcatgcatagatccacggctacataattccttttggagtcactgtcgtcgaatggc

SEQ ID NO: 24 - RNA, 100 nt, synthetic construct
  source 1..100: mol_type = other RNA
ctcagcagcagccacaactccctcgcgcaactttgaaagtggtttcatgcatagatccacggctacataattccttttggagtcactgtcgtcgaatggc

SEQ ID NO: 25 - RNA, 101 nt, synthetic construct
  source 1..101: mol_type = other RNA
ctcagcagcagccacaactccctcgcgcaactttgcacaagtcgttcatgcatagatccacggctacataattccttttggagtcactgtcgtcgaatggc

SEQ ID NO: 26 - RNA, 100 nt, synthetic construct
  source 1..100: mol_type = other RNA
ctcagcagcagccacaactccctcgcgcaactgagaaacccctttcatgcatagatccacggctacataattccttttggagtcactgtcgtcgaatggc

SEQ ID NO: 27 - RNA, 103 nt, synthetic construct
  source 1..103: mol_type = other RNA
ctcagcagcagccacaactccctcgcgcaactttgatctagtgactttcatgcatagatccacggctacataattccttttggagtcactgtcgtcgaatggc

SEQ ID NO: 28 - RNA, 100 nt, synthetic construct
  source 1..100: mol_type = other RNA
ctcagcagcagccacaactccctcgcgcaacggggaaagtccttccatgcatagatccacggctacataattccttttggagtcactgtcgtcgaatggc

SEQ ID NO: 29 - RNA, 100 nt, synthetic construct
  source 1..100: mol_type = other RNA
ctcagcagcagccacaactccctcgcgcaactttgatcgtcggttcatgcatagatccacggctacataattccttttggagtcactgtcgtcgaatggc

SEQ ID NO: 30 - RNA, 101 nt, synthetic construct
  source 1..101: mol_type = other RNA
ctcagcagcagccacaactccctcgcgcaactcatgatcctcctttcatgcatagatccacggctacataattccttttggagtcactgtcgtcgaatggc

SEQ ID NO: 31 - RNA, 100 nt, synthetic construct
  source 1..100: mol_type = other RNA
ctcagcagcagccacaactccctcgcgcaactttgaccctcctttcatgcatagaaccacggctacataattccttttggagtcactgtcgtcgaatggc

SEQ ID NO: 32 - RNA, 100 nt, synthetic construct
  source 1..100: mol_type = other RNA
ctcagcagcagccacaactccctcgcgcaactttgaaagtgacttcatgcatagatccacggctacataattccttttggagtcactgtcgtcgaatggc

SEQ ID NO: 33 - RNA, 100 nt, synthetic construct
  source 1..100: mol_type = other RNA
ctcagcagcagccacaactccctcgcgcaactttgtccgggctttcatgcatagatccacggctacataattccttttggagtcactgtcgtcgaatggc

SEQ ID NO: 34 - RNA, 100 nt, synthetic construct
  source 1..100: mol_type = other RNA
ctcagcagcagccacaactccctcgcgcaacttcataagtccgggcatgcatagatccacggctacataattccttttggagtcactgtcgtcgaatggc

SEQ ID NO: 35 - RNA, 100 nt, synthetic construct
  source 1..100: mol_type = other RNA
ctcagcagcagccacaactccctcgcgcaactcatgatgtcctttcatgcatagatccacggctacataattccttttggagtcactgtcgtcgaatggc

SEQ ID NO: 36 - RNA, 99 nt, synthetic construct
  source 1..99: mol_type = other RNA
ctcagcagcagccacaactccctcgcgcaactttgttagtccgtcatgcatagatccacggctacataattccttttggagtcactgtcgtcgaatggc

SEQ ID NO: 37 - RNA, 100 nt, synthetic construct
  source 1..100: mol_type = other RNA
ctcagcagcagccacaactccctcgcgcaacttatcaagtcggatcatgcatagatccacggctacataattccttttggagtcactgtcgtcgaatggc

SEQ ID NO: 38 - RNA, 98 nt, synthetic construct
  source 1..98: mol_type = other RNA
ctcagcagcagccacaactccctcgcgcaacttcaagtcagttcatgcatagatccacggctacataattccttttggagtcactgtcgtcgaatggc

SEQ ID NO: 39 - RNA, 98 nt, synthetic construct
  source 1..98: mol_type = other RNA
ctcagcagcagccacaactccctcgcgcaactttcagtcctttgatgcatagatccacggctacataattccttttggagtcactgtcgtcgaatggc

SEQ ID NO: 40 - RNA, 99 nt, synthetic construct
  source 1..99: mol_type = other RNA
ctcagcagcagccacaactccctcgcgcaactttgtaagtcattcatgcatagatccacggctacataattccttttggagtcactgtcgtcgaatggc

SEQ ID NO: 41 - RNA, 96 nt, synthetic construct
  source 1..96: mol_type = other RNA
ctcagcagcagccacaactccctcgcgcaactttgcgtcgtcatgcatagatccacggctacataattccttttggagtcactgtcgtcgaatggc

SEQ ID NO: 42 - RNA, 102 nt, synthetic construct
  source 1..102: mol_type = other RNA
ctcagcagcagccacaactccctcgcgcaacttgtggaaagtcgattcatgcatagatccacggctacataattccttttggagtcactgtcgtcgaatggc

SEQ ID NO: 43 - RNA, 98 nt, synthetic construct
  source 1..98: mol_type = other RNA
ctcagcagcagccacaactccctcgcgcaactttgatgtcgttcatgcatagatccacggctacataattccttttggagtcactgtcgtcgaatggc

SEQ ID NO: 44 - RNA, 102 nt, synthetic construct
  source 1..102: mol_type = other RNA
ctcagcagcagccacaactccctcgcgcaacgatgaaatcttcctttcatgcatagatccacggctacataattccttttggagtcactgtcgtcgaatggc

SEQ ID NO: 45 - RNA, 100 nt, synthetic construct
  source 1..100: mol_type = other RNA
ctcagcagcagccacaactccctcgcgcaacggtgaaagtcctttcatgcatagatctacggctacataattccttttggagtcactgtcgtcgaatggc

SEQ ID NO: 46 - RNA, 101 nt, synthetic construct
  source 1..101: mol_type = other RNA
ctcagcagcagccacaactccctcgcgcaacttatagaaagtcctgcatgcatagatccacggctacataattccttttggagtcactgtcgtcgaatggc

SEQ ID NO: 47 - RNA, 99 nt, synthetic construct
  source 1..99: mol_type = other RNA
ctcagcagcagccacaactccctcgcgcaactttgaaaggctttcatgcatagatccacggctacataattccttttggagtcactgtcgtcgaatggc

SEQ ID NO: 48 - RNA, 100 nt, synthetic construct
  source 1..100: mol_type = other RNA
ctcagcagcagccacaactccctcgcgcaactccaaaacgcctttcatgcatagatccacggctacataattccttttggagtcactgtcgtcgaatggc

SEQ ID NO: 49 - RNA, 98 nt, synthetic construct
  source 1..98: mol_type = other RNA
ctcagcagcagccacaactccctcgcgcaactttcaagtcattcatgcatagatccacggctacataattccttttggagtcactgtcgtcgaatggc

SEQ ID NO: 50 - RNA, 99 nt, synthetic construct
  source 1..99: mol_type = other RNA
ctcagcagcagccacaactccctcgcgcaacttcaaagtaatttcatgcatagatccacggctacataattccttttggagtcactgtcgtcgaatggc

SEQ ID NO: 51 - RNA, 102 nt, synthetic construct
  source 1..102: mol_type = other RNA
ctcagcagcagccacaactccctcgcgcaactttacaaaagcactttcatgcatagatccacggctacataattccttttggagtcactgtcgtcgaatggc

SEQ ID NO: 52 - RNA, 100 nt, synthetic construct
  source 1..100: mol_type = other RNA
ctcagcagcagccacaactccctcgcgcaacgaagaacttcctttcatgcatagatccacggctacataattccttttggagtcactgtcgtcgaatggc

SEQ ID NO: 53 - RNA, 101 nt, synthetic construct
  source 1..101: mol_type = other RNA
ctcagcagcagccacaactccctcgcgcaacttacaaacctcctttcatgcatagatccacggctacataattccttttggagtcactgtcgtcgaatggc

SEQ ID NO: 54 - RNA, 99 nt, synthetic construct
  source 1..99: mol_type = other RNA
ctcagcagcagccacaactccctcgcgcaacattgacgtcctttcatgcatagatccacggctacataattccttttggagtcactgtcgtcgaatggc

SEQ ID NO: 55 - RNA, 102 nt, synthetic construct
  source 1..102: mol_type = other RNA
ctcagcagcagccacaactccctcgcgcaacgctgaaagtccgggttcatgcatagatccacggctacataattccttttggagtcactgtcgtcgaatggc

SEQ ID NO: 56 - RNA, 102 nt, synthetic construct
  source 1..102: mol_type = other RNA
ctcagcagcagccacaactccctcgcgcaactttgaaagcagcagttcatgcatagatccacggctacataattccttttggagtcactgtcgtcgaatggc

SEQ ID NO: 57 - RNA, 101 nt, synthetic construct
  source 1..101: mol_type = other RNA
ctcagcagcagccacaactccctcgcgcaacttgcgaaagtaacttcatgcatagatccacggctacataattccttttggagtcactgtcgtcgaatggc

SEQ ID NO: 58 - RNA, 98 nt, synthetic construct
  source 1..98: mol_type = other RNA
ctcagcagcagccacaactccctcgcgcaactttgaaagtgttcatgcatagatccacggctacataattccttttggagtcactgtcgtcgaatggc

SEQ ID NO: 59 - RNA, 98 nt, synthetic construct
  source 1..98: mol_type = other RNA
ctcagcagcagccacaactccctcgcgcaactttgtgtcctttcatgcatagatccacggctacataattccttttggagtcactgtcgtcgaatggc

SEQ ID NO: 60 - RNA, 99 nt, synthetic construct
  source 1..99: mol_type = other RNA
ctcagcagcagccacaactccctcgcgcaacttcaagtggctttcatgcatagatccacggctacataattccttttggagtcactgtcgtcgaatggc

SEQ ID NO: 61 - RNA, 102 nt, synthetic construct
  source 1..102: mol_type = other RNA
ctcagcagcagccacaactccctcgcgcaactttgattagtccgattcatgcatagatccacggctacataattccttttggagtcactgtcgtcgaatggc

SEQ ID NO: 62 - RNA, 100 nt, synthetic construct
  source 1..100: mol_type = other RNA
ctcagcagcagccacaactccctcgcgcaacttcccaagtcctttcatgcatagatccacggctacataattccttttggagtcactgtcgtcgaatggc

SEQ ID NO: 63 - RNA, 102 nt, synthetic construct
  source 1..102: mol_type = other RNA
ctcagcagcagccacaactccctcgcgcaacattgtccaagtcctttcatgcatagatccacggctacataattccttttggagtcactgtcgtcgaatggc

SEQ ID NO: 64 - RNA, 98 nt, synthetic construct
  source 1..98: mol_type = other RNA
ctcagcagcagccacaactccctcgcgcaacttcgaaagtgttcatgcatagatccacggctacataattccttttggagtcactgtcgtcgaatggc

SEQ ID NO: 65 - RNA, 99 nt, synthetic construct
  source 1..99: mol_type = other RNA
ctcagcagcagccacaactccctcgcgcaactttgtttgtcattcatgcatagatccacggctacataattccttttggagtcactgtcgtcgaatggc

SEQ ID NO: 66 - RNA, 102 nt, synthetic construct
  source 1..102: mol_type = other RNA
ctcagcagcagccacaactccctcgcgcaactttgaaactatcctttcatgcatagatccacggctacataattccttttggagtcactgtcgtcgaatggc

SEQ ID NO: 67 - RNA, 102 nt, synthetic construct
  source 1..102: mol_type = other RNA
ctcagcagcagccacaactccctcgcgcaactttgtccaagtgctttcatgcatagatccacggctacataattccttttggagtcactgtcgtcgaatggc

SEQ ID NO: 68 - RNA, 102 nt, synthetic construct
  source 1..102: mol_type = other RNA
ctcagcagcagccacaactccctcgcgcaactttgaactcgtcctttcatgcatagatccacggctacataattccttttggagtcactgtcgtcgaatggc

SEQ ID NO: 69 - RNA, 104 nt, synthetic construct
  source 1..104: mol_type = other RNA
ctcagcagcagccacaactccctcgcgcaacttaaagaaatactcctttcatgcatagatccacggctacataattccttttggagtcactgtcgtcgaatggc

SEQ ID NO: 70 - RNA, 98 nt, synthetic construct
  source 1..98: mol_type = other RNA
ctcagcagcagccacaactccctcgcgcaacttgtaaagtccacatgcatagatccacggctacataattccttttggagtcactgtcgtcgaatggc

SEQ ID NO: 71 - RNA, 100 nt, synthetic construct
  source 1..100: mol_type = other RNA
ctcagcagcagccacaactccctcgcgcaactttgaaaggagtttcatgcatagatccacggctacataattccttttggagtcactgtcgtcgaatggc

SEQ ID NO: 72 - RNA, 100 nt, synthetic construct
  source 1..100: mol_type = other RNA
ctcagcagcagccacaactccctcgcgcaacattgaaagtcatttcatgcatagatccacggctacataattccttttggagtcactgtcgtcgaatggc

SEQ ID NO: 73 - RNA, 98 nt, synthetic construct
  source 1..98: mol_type = other RNA
ctcagcagcagccacaactccctcgcgcaactttgactgctttcatgcatagatccacggctacataattccttttggagtcactgtcgtcgaatggc

SEQ ID NO: 74 - RNA, 98 nt, synthetic construct
  source 1..98: mol_type = other RNA
ctcagcagcagccacaactccctcgcgcaactttgtgccctttcatgcatagatccacggctacataattccttttggagtcactgtcgtcgaatggc

SEQ ID NO: 75 - RNA, 100 nt, synthetic construct
  source 1..100: mol_type = other RNA
ctcagcagcagccacaactccctcgcgcaacttaaaaagtgaattcatgcatagatccacggctacataattccttttggagtcactgtcgtcgaatggc

SEQ ID NO: 76 - RNA, 99 nt, synthetic construct
  source 1..99: mol_type = other RNA
ctcagcagcagccacaactccctcgcgcaactttgaaagtcgttcatgcatagatccacggctacataattccttttggagtcactgtcgtcgaatggc

SEQ ID NO: 77 - RNA, 102 nt, synthetic construct
  source 1..102: mol_type = other RNA
ctcagcagcagccacaactccctcgcgcaacgacgaaagatacctttcatgcatagatccacggctacataattccttttggagtcactgtcgtcgaatggc

SEQ ID NO: 78 - RNA, 100 nt, synthetic construct
  source 1..100: mol_type = other RNA
ctcagcagcagccacaactccctcgcgcaacttaaaagcgcctttcatgcatagatccacggctacataattccttttggagtcactgtcgtcgaatggc

SEQ ID NO: 79 - RNA, 101 nt, synthetic construct
  source 1..101: mol_type = other RNA
ctcagcagcagccacaactccctcgcgcaacttgtaaagtccgattcatgcatagatccacggctacataattccttttggagtcactgtcgtcgaatggc

SEQ ID NO: 80 - RNA, 100 nt, synthetic construct
  source 1..100: mol_type = other RNA
ctcagcagcagccacaactccctcgcgcaacattgaatttcctttcatgcatagatccacggctacataattccttttggagtcactgtcgtcgaatggc

SEQ ID NO: 81 - RNA, 98 nt, synthetic construct
  source 1..98: mol_type = other RNA
ctcagcagcagccacaactccctcgcgcaacttttcaagtcatcatgcatagatccacggctacataattccttttggagtcactgtcgtcgaatggc

SEQ ID NO: 82 - RNA, 100 nt, synthetic construct
  source 1..100: mol_type = other RNA
ctcagcagcagccacaactccctcgcgcaactcggaaggtcctttcatgcatagatccacggctacataattccttttggagtcactgtcgtcgaatggc

SEQ ID NO: 83 - RNA, 102 nt, synthetic construct
  source 1..102: mol_type = other RNA
ctcagcagcagccacaactccctcgcgcaactttgactgtccgcgttcatgcatagatccacggctacataattccttttggagtcactgtcgtcgaatggc

SEQ ID NO: 84 - RNA, 99 nt, synthetic construct
  source 1..99: mol_type = other RNA
ctcagcagcagccacaactccctcgcgcaactttgaaagtatttcatgcatagatccacggctacataattccttttggagtcactgtcgtcgaatggc

SEQ ID NO: 85 - RNA, 101 nt, synthetic construct
  source 1..101: mol_type = other RNA
ctcagcagcagccacaactccctcgcgcaactttgaaagaacttagcatgcatagatccacggctacataattccttttggagtcactgtcgtcgaatggc

SEQ ID NO: 86 - RNA, 102 nt, synthetic construct
  source 1..102: mol_type = other RNA
ctcagcagcagccacaactccctcgcgcaacttacagaaataactttcatgcatagatccacggctacataattccttttggagtcactgtcgtcgaatggc

SEQ ID NO: 87 - RNA, 98 nt, synthetic construct
  source 1..98: mol_type = other RNA
ctcagcagcagccacaactccctcgcgcaactcggattcctttcatgcatagatccacggctacataattccttttggagtcactgtcgtcgaatggc

SEQ ID NO: 88 - RNA, 100 nt, synthetic construct
  source 1..100: mol_type = other RNA
ctcagcagcagccacaactccctcgcgcaactttctcagtgctttcatgcatagatccacggctacataattccttttggagtcactgtcgtcgaatggc

SEQ ID NO: 89 - RNA, 97 nt, synthetic construct
  source 1..97: mol_type = other RNA
ctcagcagcagccacaactccctcgcgcaacttttagtcgttcatgcatagatccacggctacataattccttttggagtcactgtcgtcgaatggc

SEQ ID NO: 90 - RNA, 100 nt, synthetic construct
  source 1..100: mol_type = other RNA
ctcagcagcagccacaactccctcgcgcaactggaaaaggagtttcatgaaccgatccacggctatcatactcctttacaccacactgtcgtcgaatggc

SEQ ID NO: 91 - RNA, 98 nt, synthetic construct
  source 1..98: mol_type = other RNA
ctcagcagcagccacaactccctcgcgcaactttgtagtcgttcatgcatagatccacggctacataattccttttggagtcactgtcgtcgaatggc

SEQ ID NO: 92 - RNA, 100 nt, synthetic construct
  source 1..100: mol_type = other RNA
ctcagcagcagccacaactccctcgcgcaacttagcttgtcctttcatgcatagatccacggctacataattccttttggagtcactgtcgtcgaatggc

SEQ ID NO: 93 - RNA, 101 nt, synthetic construct
  source 1..101: mol_type = other RNA
ctcagcagcagccacaactccctcgcgcaactttgaccagtccttgcatgcatagatccacggctacataattccttttggagtcactgtcgtcgaatggc

SEQ ID NO: 94 - RNA, 101 nt, synthetic construct
  source 1..101: mol_type = other RNA
ctcagcagcagccacaactccctcgcgcaactcatgaaattactttcatgcatagatccacggctacataattccttttggagtcactgtcgtcgaatggc

SEQ ID NO: 95 - RNA, 100 nt, synthetic construct
  source 1..100: mol_type = other RNA
ctcagcagcagccacaactccctcgcgcaacttcgaaagggctttcatgcatagatccacggctacataattccttttggagtcactgtcgtcgaatggc

SEQ ID NO: 96 - RNA, 100 nt, synthetic construct
  source 1..100: mol_type = other RNA
ctcagcagcagccacaactccctcgcgcaactttgaaagtcagttcatgcatagatccacggctacataattccttttggagtcactgtcgtcgaatggc

SEQ ID NO: 97 - RNA, 100 nt, synthetic construct
  source 1..100: mol_type = other RNA
ctcagcagcagccacaactccctcgcgcaactttgaatttaatttcatgcatagatccacggctacataattccttttggagtcactgtcgtcgaatggc

SEQ ID NO: 98 - RNA, 102 nt, synthetic construct
  source 1..102: mol_type = other RNA
ctcagcagcagccacaactccctcgcgcaacttaaaaaacttcctttcatgcatagatccacggctacataattccttttggagtcactgtcgtcgaatggc

SEQ ID NO: 99 - RNA, 97 nt, synthetic construct
  source 1..97: mol_type = other RNA
ctcagcagcagccacaactccctcgcgcaactttcgtagattcatgtcgacatccacggctatcatactcctttacaccacactgtcgtcgaatggc

SEQ ID NO: 100 - RNA, 97 nt, synthetic construct
  source 1..97: mol_type = other RNA
ctcagcagcagccacaactccctcgcgcaactttgtgtatttcatgcatagatccacggctacataattccttttggagtcactgtcgtcgaatggc

SEQ ID NO: 101 - RNA, 99 nt, synthetic construct
  source 1..99: mol_type = other RNA
ctcagcagcagccacaactccctcgcgcaacttcaaagtcctttcatgcatagatccacggctacataattccttttggagtcactgtcgtcgaatggc

SEQ ID NO: 102 - RNA, 100 nt, synthetic construct
  source 1..100: mol_type = other RNA
ctcagcagcagccacaactccctcgcgcaactttgaaactaatttcatgcatagatccacggctacataattccttttggagtcactgtcgtcgaatggc

SEQ ID NO: 103 - RNA, 98 nt, synthetic construct
  source 1..98: mol_type = other RNA
ctcagcagcagccacaactccctcgcgcaactttcagatatttcatgcatagatccacggctacataattccttttggagtcactgtcgtcgaatggc

SEQ ID NO: 104 - RNA, 100 nt, synthetic construct
  source 1..100: mol_type = other RNA
ctcagcagcagccacaactccctcgcgcaactatgaaggtcctttcatgcatagatccacggctacataattccttttggagtcactgtcgtcgaatggc

SEQ ID NO: 105 - RNA, 101 nt, synthetic construct
  source 1..101: mol_type = other RNA
ctcagcagcagccacaactccctcgcgcaacgaagaactgtcctttcatgcatagatccacggctacataattccttttggagtcactgtcgtcgaatggc

SEQ ID NO: 106 - RNA, 100 nt, synthetic construct
  source 1..100: mol_type = other RNA
ctcagcagcagccacaactccctcgcgcaactttgaaagtgctttcatgcatagatccacggctacataattccttttggagtcactgtcgtcgaatggc

SEQ ID NO: 107 - RNA, 101 nt, synthetic construct
  source 1..101: mol_type = other RNA
ctcagcagcagccacaactccctcgcgcaacttgctaaggagtttcatgatagtcatccacggctatcatactcctttacaccacactgtcgtcgaatggc

SEQ ID NO: 108 - RNA, 100 nt, synthetic construct
  source 1..100: mol_type = other RNA
ctcagcagcagccacaactccctcgcgcaacggagaaagggatttcatgacaccatccacggctatcatactcctttacaccacactgtcgtcgaatggc

SEQ ID NO: 109 - RNA, 101 nt, synthetic construct
  source 1..101: mol_type = other RNA
ctcagcagcagccacaactccctcgcgcaactttgatcagtccgggcatgcatagatccacggctacataattccttttggagtcactgtcgtcgaatggc

SEQ ID NO: 110 - RNA, 101 nt, synthetic construct
  source 1..101: mol_type = other RNA
ctcagcagcagccacaactccctcgcgcaactttgcaagtcccattcatgcatagatccacggctacataattccttttggagtcactgtcgtcgaatggc

SEQ ID NO: 111 - RNA, 102 nt, synthetic construct
  source 1..102: mol_type = other RNA
ctcagcagcagccacaactccctcgcgcaactttctcaaagtcctttcatgcatagatccacggctacataattccttttggagtcactgtcgtcgaatggc

SEQ ID NO: 112 - RNA, 100 nt, synthetic construct
  source 1..100: mol_type = other RNA
ctcagcagcagccacaactccctcgcgcaactgtgaaaatcctttcatgcatagatccacggctacataattccttttggagtcactgtcgtcgaatggc

SEQ ID NO: 113 - RNA, 99 nt, synthetic construct
  source 1..99: mol_type = other RNA
ctcagcagcagccacaactccctcgcgcaactttttaagtcattcatgcatagatccacggctacataattccttttggagtcactgtcgtcgaatggc

SEQ ID NO: 114 - RNA, 98 nt, synthetic construct
  source 1..98: mol_type = other RNA
ctcagcagcagccacaactccctcgcgcaactttgaaagatttcatgcatagatccacggctacataattccttttggagtcactgtcgtcgaatggc

SEQ ID NO: 115 - RNA, 99 nt, synthetic construct
  source 1..99: mol_type = other RNA
ctcagcagcagccacaactccctcgcgcaactttgaaagtcctggcatgcatagaccacggctacataattccttttggagtcactgtcgtcgaatggc

SEQ ID NO: 116 - RNA, 100 nt, synthetic construct
  source 1..100: mol_type = other RNA
ctcagcagcagccacaactccctcgcgcaactttgtttggggtttcatgatgtcatccacggctatcatactcctttacaccacactgtcgtcgaatggc

SEQ ID NO: 117 - RNA, 99 nt, synthetic construct
  source 1..99: mol_type = other RNA
ctcagcagcagccacaactccctcgcgcaactttccaaagtcatcatgcatagatccacggctacataattccttttggagtcactgtcgtcgaatggc

SEQ ID NO: 118 - RNA, 98 nt, synthetic construct
  source 1..98: mol_type = other RNA
ctcagcagcagccacaactccctcgcgcaactttgattcatttcatgcatagatccacggctacataattccttttggagtcactgtcgtcgaatggc

SEQ ID NO: 119 - RNA, 103 nt, synthetic construct
  source 1..103: mol_type = other RNA
ctcagcagcagccacaactccctcgcgcaacttgtcgaaagtcctttcatgcatagatggcacggctacataattccttttggagtcactgtcgtcgaatggc

SEQ ID NO: 120 - RNA, 101 nt, synthetic construct
  source 1..101: mol_type = other RNA
ctcagcagcagccacaactccctcgcgcaactagctaaaggagtttcatgaatagccccacggctatcatactcctttacaccacactgtcgtcgaatggc

SEQ ID NO: 121 - RNA, 100 nt, synthetic construct
  source 1..100: mol_type = other RNA
ctcagcagcagccacaactccctcgcgcaacgctgaaagtcctttcatgcatagatccacggctacataattccttttggagtcactgtcgtcgaatggc

SEQ ID NO: 122 - RNA, 100 nt, synthetic construct
  source 1..100: mol_type = other RNA
ctcagcagcagccacaactccctcgcgcaactgccaaaggagtttcatgtacccatccacggctatcatactcctttacaccacactgtcgtcgaatggc

SEQ ID NO: 123 - RNA, 98 nt, synthetic construct
  source 1..98: mol_type = other RNA
ctcagcagcagccacaactccctcgcgcaacttcaagtcctttcatgcatagatccacggctacataattccttttggagtcactgtcgtcgaatggc

SEQ ID NO: 124 - RNA, 100 nt, synthetic construct
  source 1..100: mol_type = other RNA
ctcagcagcagccacaactccctcgcgcaactttgaaagacacatcatgcatagatccacggctacataattccttttggagtcactgtcgtcgaatggc

SEQ ID NO: 125 - RNA, 100 nt, synthetic construct
  source 1..100: mol_type = other RNA
ctcagcagcagccacaactccctcgcgcaactacataaggagtttcatcgttacatccacggctatcatactcctttacaccacactgtcgtcgaatggc

SEQ ID NO: 126 - RNA, 102 nt, synthetic construct
  source 1..102: mol_type = other RNA
ctcagcagcagccacaactccctcgcgcaactttgaaataatcacttcatgcatagatccacggctacataattccttttggagtcactgtcgtcgaatggc

SEQ ID NO: 127 - RNA, 101 nt, synthetic construct
  source 1..101: mol_type = other RNA
ctcagcagcagccacaactccctcgcgcaactttgaaagcaccaatcatgcatagatccacggctacataattccttttggagtcactgtcgtcgaatggc

SEQ ID NO: 128 - RNA, 102 nt, synthetic construct
  source 1..102: mol_type = other RNA
ctcagcagcagccacaactccctcgcgcaactttgaactgtaactttcatgcatagatccacggctacataattccttttggagtcactgtcgtcgaatggc

SEQ ID NO: 129 - RNA, 100 nt, synthetic construct
  source 1..100: mol_type = other RNA
ctcagcagcagccacaactccctcgcgcaacttcgaaacgcctttcatgcatagatccacggctacataattccttttggagtcactgtcgtcgaatggc

SEQ ID NO: 130 - RNA, 97 nt, synthetic construct
  source 1..97: mol_type = other RNA
ctcagcagcagccacaactccctcgcgcaactttgtagatttcatgcatagatccacggctacataattccttttggagtcactgtcgtcgaatggc

SEQ ID NO: 131 - RNA, 102 nt, synthetic construct
  source 1..102: mol_type = other RNA
ctcagcagcagccacaactccctcgcgcaactttgaatcgtccgattcatgcatagatccacggctacataattccttttggagtcactgtcgtcgaatggc

SEQ ID NO: 132 - RNA, 102 nt, synthetic construct
  source 1..102: mol_type = other RNA
ctcagcagcagccacaactccctcgcgcaactttgactatggggtttcatccttacatccacggctatcatactcctttacaccacactgtcgtcgaatggc

SEQ ID NO: 133 - RNA, 101 nt, synthetic construct
  source 1..101: mol_type = other RNA
ctcagcagcagccacaactccctcgcgcaactttgctaaaccctttcatgcatagatccacggctacataattccttttggagtcactgtcgtcgaatggc

SEQ ID NO: 134 - RNA, 100 nt, synthetic construct
  source 1..100: mol_type = other RNA
ctcagcagcagccacaactccctcgcgcaactgccaaaggagtttcatgcgcacatccacggctatcatactcctttacaccacactgtcgtcgaatggc

SEQ ID NO: 135 - RNA, 102 nt, synthetic construct
  source 1..102: mol_type = other RNA
ctcagcagcagccacaactccctcgcgcaacttaacaagcggtttcatgtcgctacatccacggctatcatactcctttacaccacactgtcgtcgaatggc

SEQ ID NO: 136 - RNA, 99 nt, synthetic construct
  source 1..99: mol_type = other RNA
ctcagcagcagccacaactccctcgcgcaactttgaaagtcattcatgcatagacacacggctacataattccttttggagtcactgtcgtcgaatggc

SEQ ID NO: 137 - RNA, 98 nt, synthetic construct
  source 1..98: mol_type = other RNA
ctcagcagcagccacaactccctcgcgcaactttgactcacatcatgcatagatccacggctacataattccttttggagtcactgtcgtcgaatggc

SEQ ID NO: 138 - RNA, 100 nt, synthetic construct
  source 1..100: mol_type = other RNA
ctcagcagcagccacaactccctcgcgcaactttgaaagtgatttcatgaatagatccacggctatcatactcctttacaccacactgtcgtcgaatggc

SEQ ID NO: 139 - RNA, 102 nt, synthetic construct
  source 1..102: mol_type = other RNA
ctcagcagcagccacaactccctcgcgcaacggggaaagtagtatttcatgatgccatccacggctatcatactcctttacaccacactgtcgtcgaatggc

SEQ ID NO: 140 - RNA, 101 nt, synthetic construct
  source 1..101: mol_type = other RNA
ctcagcagcagccacaactccctcgcgcaacttctctaagtgggttcatgacctcatccacggctatcatactcctttacaccacactgtcgtcgaatggc

SEQ ID NO: 141 - RNA, 98 nt, synthetic construct
  source 1..98: mol_type = other RNA
ctcagcagcagccacaactccctcgcgcaactgtgaaagtcgtcatgcttacatccacggctatcatactcctttacaccacactgtcgtcgaatggc

SEQ ID NO: 142 - RNA, 98 nt, synthetic construct
  source 1..98: mol_type = other RNA
ctcagcagcagccacaactccctcgcgcaactttgtagtcattcatgcatagatccacggctacataattccttttggagtcactgtcgtcgaatggc

SEQ ID NO: 143 - RNA, 101 nt, synthetic construct
  source 1..101: mol_type = other RNA
ctcagcagcagccacaactccctcgcgcaacgaagaacccgcctttcatgctgacatccacggctatcatactcctttacaccacactgtcgtcgaatggc

SEQ ID NO: 144 - RNA, 102 nt, synthetic construct
  source 1..102: mol_type = other RNA
ctcagcagcagccacaactccctcgcgcaacttctataaaggaatttcatgaataaccccacggctatcatactcctttacaccacactgtcgtcgaatggc

SEQ ID NO: 145 - RNA, 99 nt, synthetic construct
  source 1..99: mol_type = other RNA
ctcagcagcagccacaactccctcgcgcaactttgaaaggggtttcatgcatagaccacggctacataattccttttggagtcactgtcgtcgaatggc

SEQ ID NO: 146 - RNA, 100 nt, synthetic construct
  source 1..100: mol_type = other RNA
ctcagcagcagccacaactccctcgcgcaactttgattgtcctgtcatgcatagatccacggctacataattccttttggagtcactgtcgtcgaatggc

SEQ ID NO: 147 - RNA, 96 nt, synthetic construct
  source 1..96: mol_type = other RNA
ctcagcagcagccacaactccctcgcgcaactttgcgtcctgatgcatagatccacggctacataattccttttggagtcactgtcgtcgaatggc

SEQ ID NO: 148 - RNA, 100 nt, synthetic construct
  source 1..100: mol_type = other RNA
ctcagcagcagccacaactccctcgcgcaactcgaaaagtgacttcattgctacatccacggctatcatactcctttacaccacactgtcgtcgaatggc

SEQ ID NO: 149 - RNA, 99 nt, synthetic construct
  source 1..99: mol_type = other RNA
ctcagcagcagccacaactccctcgcgcaactttgaaagtaacttcatgcatagaccacggctacataattccttttggagtcactgtcgtcgaatggc

SEQ ID NO: 150 - RNA, 99 nt, synthetic construct
  source 1..99: mol_type = other RNA
ctcagcagcagccacaactccctcgcgcaactttgaaagtggtttcatgcatagaccacggctacataattccttttggagtcactgtcgtcgaatggc

SEQ ID NO: 151 - RNA, 100 nt, synthetic construct
  source 1..100: mol_type = other RNA
ctcagcagcagccacaactccctcgcgcaactgtgaaagtcctttcatgcatagatccacggctacataattccttttggagtcactgtcgtcgaatggc

SEQ ID NO: 152 - RNA, 101 nt, synthetic construct
  source 1..101: mol_type = other RNA
ctcagcagcagccacaactccctcgcgcaactttgctaagtcctgtcatgcatagatccacggctacataattccttttggagtcactgtcgtcgaatggc

SEQ ID NO: 153 - RNA, 101 nt, synthetic construct
  source 1..101: mol_type = other RNA
ctcagcagcagccacaactccctcgcgcaactttctcaaagtccgtcatgactacatccacggctatcatactcctttacaccacactgtcgtcgaatggc

SEQ ID NO: 154 - RNA, 102 nt, synthetic construct
  source 1..102: mol_type = other RNA
ctcagcagcagccacaactccctcgcgcaactttgaccctccgcgttcatgcatagatccacggctacataattccttttggagtcactgtcgtcgaatggc

SEQ ID NO: 155 - RNA, 99 nt, synthetic construct
  source 1..99: mol_type = other RNA
ctcagcagcagccacaactccctcgcgcaactttgaaaggactttcatgcatagaccacggctacataattccttttggagtcactgtcgtcgaatggc

SEQ ID NO: 156 - RNA, 102 nt, synthetic construct
  source 1..102: mol_type = other RNA
ctcagcagcagccacaactccctcgcgcaactttgtttaagtcctctcatgcatagatccacggctacataattccttttggagtcactgtcgtcgaatggc

SEQ ID NO: 157 - RNA, 99 nt, synthetic construct
  source 1..99: mol_type = other RNA
ctcagcagcagccacaactccctcgcgcaactttgacgctccctcatgcatagatccacggctacataattccttttggagtcactgtcgtcgaatggc

SEQ ID NO: 158 - RNA, 100 nt, synthetic construct
  source 1..100: mol_type = other RNA
ctcagcagcagccacaactccctcgcgcaactttgacgcagtcgtcatgcatagatccacggctacataattccttttggagtcactgtcgtcgaatggc

SEQ ID NO: 159 - RNA, 98 nt, synthetic construct
  source 1..98: mol_type = other RNA
ctcagcagcagccacaactccctcgcgcaactttgaaagtgttcgtgcatagatccacggctacataattccttttggagtcactgtcgtcgaatggc

SEQ ID NO: 160 - RNA, 101 nt, synthetic construct
  source 1..101: mol_type = other RNA
ctcagcagcagccacaactccctcgcgcaactttgtcaagtccgttcatgcatagatccacggctacataattccttttggagtcactgtcgtcgaatggc

SEQ ID NO: 161 - RNA, 97 nt, synthetic construct
  source 1..97: mol_type = other RNA
ctcagcagcagccacaactccctcgcgcaacttcaagtgtttcatgaatcgatccacggctatcatactcctttacaccacactgtcgtcgaatggc

SEQ ID NO: 162 - RNA, 99 nt, synthetic construct
  source 1..99: mol_type = other RNA
ctcagcagcagccacaactccctcgcgcaactttgaaagtgtttcatgcatacaaccacggctatcatactcctttacaccacactgtcgtcgaatggc

SEQ ID NO: 163 - RNA, 100 nt, synthetic construct
  source 1..100: mol_type = other RNA
ctcagcagcagccacaactccctcgcgcaactttgaccgtcctttcatgcatagatccacggctacataattccttttggagtcactgtcgtcgaatggc

SEQ ID NO: 164 - RNA, 102 nt, synthetic construct
  source 1..102: mol_type = other RNA
ctcagcagcagccacaactccctcgcgcaactttgaaattcccgattcatgcatagatccacggctacataattccttttggagtcactgtcgtcgaatggc

SEQ ID NO: 165 - RNA, 100 nt, synthetic construct
  source 1..100: mol_type = other RNA
ctcagcagcagccacaactccctcgcgcaactacaaaaggggattcatccttacatccacggctatcatactcctttacaccacactgtcgtcgaatggc

SEQ ID NO: 166 - RNA, 100 nt, synthetic construct
  source 1..100: mol_type = other RNA
ctcagcagcagccacaactccctcgcgcaactttgcttggatgttcattgctacatccacggctatcatactcctttacaccacactgtcgtcgaatggc

SEQ ID NO: 167 - RNA, 98 nt, synthetic construct
  source 1..98: mol_type = other RNA
ctcagcagcagccacaactccctcgcgcaactttgaaaggtttcatgaatagatctacggctatcatactcctttacaccacactgtcgtcgaatggc

SEQ ID NO: 168 - RNA, 101 nt, synthetic construct
  source 1..101: mol_type = other RNA
ctcagcagcagccacaactccctcgcgcaactttgaaacagcttcccatgcatagatccacggctacataattccttttggagtcactgtcgtcgaatggc

SEQ ID NO: 169 - RNA, 99 nt, synthetic construct
  source 1..99: mol_type = other RNA
ctcagcagcagccacaactccctcgcgcaactttgaaagtccttaattgcatagaccacggctacataattccttttggagtcactgtcgtcgaatggc

SEQ ID NO: 170 - RNA, 99 nt, synthetic construct
  source 1..99: mol_type = other RNA
ctcagcagcagccacaactccctcgcgcaactttgtgtgatttcatgaataccctccacggctatcatactcctttacaccacactgtcgtcgaatggc

SEQ ID NO: 171 - RNA, 103 nt, synthetic construct
  source 1..103: mol_type = other RNA
ctcagcagcagccacaactccctcgcgcaacgtcgaccctcctttcatgaattgaccatccacggctatcatactcctttacaccacactgtcgtcgaatggc

SEQ ID NO: 172 - RNA, 98 nt, synthetic construct
  source 1..98: mol_type = other RNA
ctcagcagcagccacaactccctcgcgcaactttgaaagtattcatgcatagatctacggctacataattccttttggagtcactgtcgtcgaatggc

SEQ ID NO: 173 - RNA, 99 nt, synthetic construct
  source 1..99: mol_type = other RNA
ctcagcagcagccacaactccctcgcgcaacggtgaaagacctttcatgaatacaccacggctatcatactcctttacaccacactgtcgtcgaatggc

SEQ ID NO: 174 - RNA, 98 nt, synthetic construct
  source 1..98: mol_type = other RNA
ctcagcagcagccacaactccctcgcgcaactttcagtgatttcatgaatagatccacggctatcatactcctttacaccacactgtcgtcgaatggc

SEQ ID NO: 175 - RNA, 98 nt, synthetic construct
  source 1..98: mol_type = other RNA
ctcagcagcagccacaactccctcgcgcaactttgaaccctggaatgcatagatccacggctacataattccttttggagtcactgtcgtcgaatggc

SEQ ID NO: 176 - RNA, 99 nt, synthetic construct
  source 1..99: mol_type = other RNA
ctcagcagcagccacaactccctcgcgcaactttgtagtcctttcatgcatagatccacggctacataattccttttggagtcactgtcgtcgaatggc

SEQ ID NO: 177 - RNA, 100 nt, synthetic construct
  source 1..100: mol_type = other RNA
ctcagcagcagccacaactccctcgcgcaactttgaaagtgatttcatgcatagacacacggctacataattccttttggagtcactgtcgtcgaatggc

SEQ ID NO: 178 - RNA, 98 nt, synthetic construct
  source 1..98: mol_type = other RNA
ctcagcagcagccacaactccctcgcgcaactttgactcctagcatgcatagatccacggctacataattccttttggagtcactgtcgtcgaatggc

SEQ ID NO: 179 - RNA, 102 nt, synthetic construct
  source 1..102: mol_type = other RNA
ctcagcagcagccacaactccctcgcgcaactgccaaagtggggtttcatgtccacatccacggctatcatactcctttacaccacactgtcgtcgaatggc

SEQ ID NO: 180 - RNA, 97 nt, synthetic construct
  source 1..97: mol_type = other RNA
ctcagcagcagccacaactccctcgcgcaactttgaaagtcgtcatgttacaaccacggctatcatactcctttacaccacactgtcgtcgaatggc

SEQ ID NO: 181 - RNA, 101 nt, synthetic construct
  source 1..101: mol_type = other RNA
ctcagcagcagccacaactccctcgcgcaactttgttaagtcctttcgtgcatagatccacggctacataattccttttggagtcactgtcgtcgaatggc

SEQ ID NO: 182 - RNA, 99 nt, synthetic construct
  source 1..99: mol_type = other RNA
ctcagcagcagccacaactccctcgcgcaactttgtagtcgtttcatgcatagatccacggctacataattccttttggagtcactgtcgtcgaatggc

SEQ ID NO: 183 - RNA, 100 nt, synthetic construct
  source 1..100: mol_type = other RNA
ctcagcagcagccacaactccctcgcgcaactttgaaagtattccatgaataagatccacggctatcatactcctttacaccacactgtcgtcgaatggc

SEQ ID NO: 184 - RNA, 99 nt, synthetic construct
  source 1..99: mol_type = other RNA
ctcagcagcagccacaactccctcgcgcaactttgaaaggagtttcatgcatagaccacggctacataattccttttggagtcactgtcgtcgaatggc

SEQ ID NO: 185 - RNA, 101 nt, synthetic construct
  source 1..101: mol_type = other RNA
ctcagcagcagccacaactccctcgcgcaacttgttaagcatatttcatcactacatccacggctatcatactcctttacaccacactgtcgtcgaatggc

SEQ ID NO: 186 - RNA, 100 nt, synthetic construct
  source 1..100: mol_type = other RNA
ctcagcagcagccacaactccctcgcgcaacttgtcaagagatttcatctttacatccacggctatcatactcctttacaccacactgtcgtcgaatggc

SEQ ID NO: 187 - RNA, 98 nt, synthetic construct
  source 1..98: mol_type = other RNA
ctcagcagcagccacaactccctcgcgcaactttgaaaggctttcatgaatgcaccacggctatcatactcctttacaccacactgtcgtcgaatggc

SEQ ID NO: 188 - RNA, 99 nt, synthetic construct
  source 1..99: mol_type = other RNA
ctcagcagcagccacaactccctcgcgcaactttgaactacctacatgcatagatccacggctacataattccttttggagtcactgtcgtcgaatggc

SEQ ID NO: 189 - RNA, 102 nt, synthetic construct
  source 1..102: mol_type = other RNA
ctcagcagcagccacaactccctcgcgcaactttgcataagcactttcatgcatagatccacggctacataattccttttggagtcactgtcgtcgaatggc

SEQ ID NO: 190 - RNA, 103 nt, synthetic construct
  source 1..103: mol_type = other RNA
ctcagcagcagccacaactccctcgcgcaactttgaatgctgtggattcatgctcacatccacggctatcatactcctttacaccacactgtcgtcgaatggc

SEQ ID NO: 191 - RNA, 99 nt, synthetic construct
  source 1..99: mol_type = other RNA
ctcagcagcagccacaactccctcgcgcaactttgaaagtccagtcatgcatagaccacggctacataattccttttggagtcactgtcgtcgaatggc

SEQ ID NO: 192 - RNA, 100 nt, synthetic construct
  source 1..100: mol_type = other RNA
ctcagcagcagccacaactccctcgcgcaactttgacagccctttcatgcatagatccacggctacataattccttttggagtcactgtcgtcgaatggc

SEQ ID NO: 193 - RNA, 100 nt, synthetic construct
  source 1..100: mol_type = other RNA
ctcagcagcagccacaactccctcgcgcaactgtccaaggaatttcacttatacatccacggctatcatactcctttacaccacactgtcgtcgaatggc

SEQ ID NO: 194 - RNA, 99 nt, synthetic construct
  source 1..99: mol_type = other RNA
ctcagcagcagccacaactccctcgcgcaactttgaaagtcattcatgaatccatccacggctatcatactcctttacaccacactgtcgtcgaatggc

SEQ ID NO: 195 - RNA, 100 nt, synthetic construct
  source 1..100: mol_type = other RNA
ctcagcagcagccacaactccctcgcgcaacttcctaagcgggttcatagttacatccacggctatcatactcctttacaccacactgtcgtcgaatggc

SEQ ID NO: 196 - RNA, 98 nt, synthetic construct
  source 1..98: mol_type = other RNA
ctcagcagcagccacaactccctcgcgcaactttgaaagtcgttcatgaatgcaccacggctatcatactcctttacaccacactgtcgtcgaatggc

SEQ ID NO: 197 - RNA, 100 nt, synthetic construct
  source 1..100: mol_type = other RNA
ctcagcagcagccacaactccctcgcgcaactttgaaagtcattcatgaataagatccacggctatcatactcctttacaccacactgtcgtcgaatggc

SEQ ID NO: 198 - RNA, 101 nt, synthetic construct
  source 1..101: mol_type = other RNA
ctcagcagcagccacaactccctcgcgcaactttgaaagacccctttcatgcatagaccacggctacataattccttttggagtcactgtcgtcgaatggc

SEQ ID NO: 199 - RNA, 100 nt, synthetic construct
  source 1..100: mol_type = other RNA
ctcagcagcagccacaactccctcgcgcaacgacgaaaggtatttcatgattccatccacggctatcatactcctttacaccacactgtcgtcgaatggc

SEQ ID NO: 200 - RNA, 100 nt, synthetic construct
  source 1..100: mol_type = other RNA
ctcagcagcagccacaactccctcgcgcaactttgactgtatttcatgccatacatccacggctatcatactcctttacaccacactgtcgtcgaatggc

SEQ ID NO: 201 - RNA, 100 nt, synthetic construct
  source 1..100: mol_type = other RNA
ctcagcagcagccacaactccctcgcgcaactgctaaagcgatttcattgctacatccacggctatcatactcctttacaccacactgtcgtcgaatggc

SEQ ID NO: 202 - RNA, 99 nt, synthetic construct
  source 1..99: mol_type = other RNA
ctcagcagcagccacaactccctcgcgcaactttgaaatgactttcatgcatagaccacggctacataattccttttggagtcactgtcgtcgaatggc

SEQ ID NO: 203 - RNA, 99 nt, synthetic construct
  source 1..99: mol_type = other RNA
ctcagcagcagccacaactccctcgcgcaactttgaaagtatttcatgacgacaaccacggctatcatactcctttacaccacactgtcgtcgaatggc

SEQ ID NO: 204 - RNA, 100 nt, synthetic construct
  source 1..100: mol_type = other RNA
ctcagcagcagccacaactccctcgcgcaactttgtctgtcctttcatgcatagatccacggctacataattccttttggagtcactgtcgtcgaatggc

SEQ ID NO: 205 - RNA, 99 nt, synthetic construct
  source 1..99: mol_type = other RNA
ctcagcagcagccacaactccctcgcgcaactttgaaactactttcatgcatagaccacggctacataattccttttggagtcactgtcgtcgaatggc

SEQ ID NO: 206 - RNA, 100 nt, synthetic construct
  source 1..100: mol_type = other RNA
ctcagcagcagccacaactccctcgcgcaactttgccagtcctttcatgcatagacccacggctacataattccttttggagtcactgtcgtcgaatggc

SEQ ID NO: 207 - RNA, 97 nt, synthetic construct
  source 1..97: mol_type = other RNA
ctcagcagcagccacaactccctcgcgcaactttgtttcctttcatgtgcccatccacggcaatcgccgactttacaccacactgtcgtcgaatggc

SEQ ID NO: 208 - RNA, 98 nt, synthetic construct
  source 1..98: mol_type = other RNA
ctcagcagcagccacaactccctcgcgcaactttgaaaactttcatgcatagatcgacggctacataattccttttggagtcactgtcgtcgaatggc

SEQ ID NO: 209 - RNA, 99 nt, synthetic construct
  source 1..99: mol_type = other RNA
ctcagcagcagccacaactccctcgcgcaactttgaaagtcattcatgactacatccacggctatcatactcctttacaccacactgtcgtcgaatggc

SEQ ID NO: 210 - RNA, 100 nt, synthetic construct
  source 1..100: mol_type = other RNA
ctcagcagcagccacaactccctcgcgcaacttcaagtaagctttcatgcatacatccacggctatcatactcctttacaccacactgtcgtcgaatggc

SEQ ID NO: 211 - RNA, 99 nt, synthetic construct
  source 1..99: mol_type = other RNA
ctcagcagcagccacaactccctcgcgcaactttgaccacctttcatgctacacatccacggcaatcgccgactttacaccacactgtcgtcgaatggc

SEQ ID NO: 212 - RNA, 99 nt, synthetic construct
  source 1..99: mol_type = other RNA
ctcagcagcagccacaactccctcgcgcaactttgaaagtaatttcatgcatagaccacggctacataattccttttggagtcactgtcgtcgaatggc

SEQ ID NO: 213 - RNA, 100 nt, synthetic construct
  source 1..100: mol_type = other RNA
ctcagcagcagccacaactccctcgcgcaactcatgatgtcctttcatgaatacatctacggctatcatactcctttacaccacactgtcgtcgaatggc

SEQ ID NO: 214 - RNA, 98 nt, synthetic construct
  source 1..98: mol_type = other RNA
ctcagcagcagccacaactccctcgcgcaactttgaaagtcgttcatgaaccatccacggctatcatactcctttacaccacactgtcgtcgaatggc

SEQ ID NO: 215 - RNA, 100 nt, synthetic construct
  source 1..100: mol_type = other RNA
ctcagcagcagccacaactccctcgcgcaactctaaaagtgggttcatgcggccatccacggctatcatactcctttacaccacactgtcgtcgaatggc

SEQ ID NO: 216 - RNA, 97 nt, synthetic construct
  source 1..97: mol_type = other RNA
ctcagcagcagccacaactccctcgcgcaactttgaacgctttcctccatacatccacggcaatcgccgactttacaccacactgtcgtcgaatggc

SEQ ID NO: 217 - RNA, 97 nt, synthetic construct
  source 1..97: mol_type = other RNA
ctcagcagcagccacaactccctcgcgcaacttcccaagattcatccttacatccacggctatcatactcctttacaccacactgtcgtcgaatggc

SEQ ID NO: 218 - RNA, 101 nt, synthetic construct
  source 1..101: mol_type = other RNA
ctcagcagcagccacaactccctcgcgcaactttgtcaagtactttcatgcatagatccacggctacataattccttttggagtcactgtcgtcgaatggc

SEQ ID NO: 219 - RNA, 99 nt, synthetic construct
  source 1..99: mol_type = other RNA
ctcagcagcagccacaactccctcgcgcaactttgaaagtccaggcatgcatagaccacggctacataattccttttggagtcactgtcgtcgaatggc

SEQ ID NO: 220 - RNA, 99 nt, synthetic construct
  source 1..99: mol_type = other RNA
ctcagcagcagccacaactccctcgcgcaactttgaaagtgatttcatgcatagaccacggctacataattccttttggagtcactgtcgtcgaatggc

SEQ ID NO: 221 - RNA, 99 nt, synthetic construct
  source 1..99: mol_type = other RNA
ctcagcagcagccacaactccctcgcgcaactttgaaagatttcatgacttacttccacggctatcatactcctttacaccacactgtcgtcgaatggc

SEQ ID NO: 222 - RNA, 98 nt, synthetic construct
  source 1..98: mol_type = other RNA
ctcagcagcagccacaactccctcgcgcaactttgaaagtgtttcatgaaaccaccacggctatcatactcctttacaccacactgtcgtcgaatggc

SEQ ID NO: 223 - RNA, 100 nt, synthetic construct
  source 1..100: mol_type = other RNA
ctcagcagcagccacaactccctcgcgcaactttcttagtaggttcatgaatctctccacggctatcatactcctttacaccacactgtcgtcgaatggc

SEQ ID NO: 224 - RNA, 103 nt, synthetic construct
  source 1..103: mol_type = other RNA
ctcagcagcagccacaactccctcgcgcaactttgaattgcgtgggttcattgttacatccacggctatcatactcctttacaccacactgtcgtcgaatggc

SEQ ID NO: 225 - RNA, 99 nt, synthetic construct
  source 1..99: mol_type = other RNA
ctcagcagcagccacaactccctcgcgcaactttgactgtcgttcatgaatccatccacggctatcatactcctttacaccacactgtcgtcgaatggc

SEQ ID NO: 226 - RNA, 100 nt, synthetic construct
  source 1..100: mol_type = other RNA
ctcagcagcagccacaactccctcgcgcaactgttaaagtcctttcactcatacatctacggctatcatactcctttacaccacactgtcgtcgaatggc

SEQ ID NO: 227 - RNA, 101 nt, synthetic construct
  source 1..101: mol_type = other RNA
ctcagcagcagccacaactccctcgcgcaacgtcgattactcctttcatgaataaagccacggctatcatactcctttacaccacactgtcgtcgaatggc

SEQ ID NO: 228 - RNA, 100 nt, synthetic construct
  source 1..100: mol_type = other RNA
ctcagcagcagccacaactccctcgcgcaactttgaaagggctttcatgaatcgatccacggctatcatactcctttacaccacactgtcgtcgaatggc

SEQ ID NO: 229 - RNA, 98 nt, synthetic construct
  source 1..98: mol_type = other RNA
ctcagcagcagccacaactccctcgcgcaactttgaaagtgtttcatgaaccatccacggctatcatactcctttacaccacactgtcgtcgaatggc

SEQ ID NO: 230 - RNA, 102 nt, synthetic construct
  source 1..102: mol_type = other RNA
ctcagcagcagccacaactccctcgcgcaacgaagaacaacctttcatgtgtttacatccacggctatcatactcctttacaccacactgtcgtcgaatggc

SEQ ID NO: 231 - RNA, 101 nt, synthetic construct
  source 1..101: mol_type = other RNA
ctcagcagcagccacaactccctcgcgcaactttgttaagactttcatgaatactctccacggctatcatactcctttacaccacactgtcgtcgaatggc

SEQ ID NO: 232 - RNA, 100 nt, synthetic construct
  source 1..100: mol_type = other RNA
ctcagcagcagccacaactccctcgcgcaactttgatcgtcctttgatgaatccatccacggctatcatactcctttacaccacactgtcgtcgaatggc

SEQ ID NO: 233 - RNA, 97 nt, synthetic construct
  source 1..97: mol_type = other RNA
ctcagcagcagccacaactccctcgcgcaacttttagtatttcatgaatacaaccacggctatcatactcctttacaccacactgtcgtcgaatggc

SEQ ID NO: 234 - RNA, 100 nt, synthetic construct
  source 1..100: mol_type = other RNA
ctcagcagcagccacaactccctcgcgcaactttgaaagtgtttcatgaatactttccacggctatcatactcctttacaccacactgtcgtcgaatggc

SEQ ID NO: 235 - RNA, 99 nt, synthetic construct
  source 1..99: mol_type = other RNA
ctcagcagcagccacaactccctcgcgcaactttgaaagactttcatgaatccatccacggctatcatactcctttacaccacactgtcgtcgaatggc

SEQ ID NO: 236 - RNA, 98 nt, synthetic construct
  source 1..98: mol_type = other RNA
ctcagcagcagccacaactccctcgcgcaacttcctaagggttcatccttacatccacggctatcatactcctttacaccacactgtcgtcgaatggc

SEQ ID NO: 237 - RNA, 100 nt, synthetic construct
  source 1..100: mol_type = other RNA
ctcagcagcagccacaactccctcgcgcaactttgaaagtccgttcatgcatagagccacggctacataattccttttggagtcactgtcgtcgaatggc

SEQ ID NO: 238 - RNA, 100 nt, synthetic construct
  source 1..100: mol_type = other RNA
ctcagcagcagccacaactccctcgcgcaacattgaaagtcctttcatgaatagtttcacggctatcatactcctttacaccacactgtcgtcgaatggc

SEQ ID NO: 239 - RNA, 100 nt, synthetic construct
  source 1..100: mol_type = other RNA
ctcagcagcagccacaactccctcgcgcaacttataacctcctttcatgaatagatccacggctatcatactcctttacaccacactgtcgtcgaatggc

SEQ ID NO: 240 - RNA, 98 nt, synthetic construct
  source 1..98: mol_type = other RNA
ctcagcagcagccacaactccctcgcgcaactttgatcgtcctttcatctacatctacggctatcatactcctttacaccacactgtcgtcgaatggc

SEQ ID NO: 241 - RNA, 98 nt, synthetic construct
  source 1..98: mol_type = other RNA
ctcagcagcagccacaactccctcgcgcaactttgaccctttcatgtctttacatccacggcaatcgccgactttacaccacactgtcgtcgaatggc

SEQ ID NO: 242 - RNA, 99 nt, synthetic construct
  source 1..99: mol_type = other RNA
ctcagcagcagccacaactccctcgcgcaactttgaaagtcatctcatgcatagaccacggctacataattccttttggagtcactgtcgtcgaatggc

SEQ ID NO: 243 - RNA, 104 nt, synthetic construct
  source 1..104: mol_type = other RNA
ctcagcagcagccacaactccctcgcgcaacgatgatgcagtcctttcatgaataaagatccacggctatcatactcctttacaccacactgtcgtcgaatggc

SEQ ID NO: 244 - RNA, 100 nt, synthetic construct
  source 1..100: mol_type = other RNA
ctcagcagcagccacaactccctcctctcgagttgaaagagctttcatgaatacaaccacggctacggatagagcttacaccacactgtcgtcgaatggc

SEQ ID NO: 245 - RNA, 102 nt, synthetic construct
  source 1..102: mol_type = other RNA
ctcagcagcagccacaactccctcgcgcaactttgatgacgtgggttcatctttacatccacggctatcatactcctttacaccacactgtcgtcgaatggc

SEQ ID NO: 246 - RNA, 99 nt, synthetic construct
  source 1..99: mol_type = other RNA
ctcagcagcagccacaactccctcgcgcaactttgaaagtatttcatgaatacagccacggctatcatactcctttacaccacactgtcgtcgaatggc

SEQ ID NO: 247 - RNA, 98 nt, synthetic construct
  source 1..98: mol_type = other RNA
ctcagcagcagccacaactccctcctatagagttgaaaggtttcatcaatacatctacggctaatcgccgactttacaccacactgtcgtcgaatggc

SEQ ID NO: 248 - RNA, 98 nt, synthetic construct
  source 1..98: mol_type = other RNA
ctcagcagcagccacaactccctcgcgcaactttgaaaggtttcatgaacccatccacggctatcatactcctttacaccacactgtcgtcgaatggc

SEQ ID NO: 249 - RNA, 98 nt, synthetic construct
  source 1..98: mol_type = other RNA
ctcagcagcagccacaactccctcgcgcaactttgaaaggtttcatgaatacagccacggctatcatactcctttacaccacactgtcgtcgaatggc

SEQ ID NO: 250 - RNA, 100 nt, synthetic construct
  source 1..100: mol_type = other RNA
ctcagcagcagccacaactccctcgcgcaacattgaaagtatttcatgaatacatagcacggctatcatactcctttacaccacactgtcgtcgaatggc

SEQ ID NO: 251 - RNA, 100 nt, synthetic construct
  source 1..100: mol_type = other RNA
ctcagcagcagccacaactccctcgcgcaactttgaaagtccagtcatgataacatccacggctatcatactcctttacaccacactgtcgtcgaatggc

SEQ ID NO: 252 - RNA, 99 nt, synthetic construct
  source 1..99: mol_type = other RNA
ctcagcagcagccacaactccctcctctcgagttgaaagtgtttcatgcttacatccacggctacggatagagcttacaccacactgtcgtcgaatggc

SEQ ID NO: 253 - RNA, 97 nt, synthetic construct
  source 1..97: mol_type = other RNA
ctcagcagcagccacaactccctcgcgcaactttgatgcctttcattataacatccacggcaatcgccgactttacaccacactgtcgtcgaatggc

SEQ ID NO: 254 - RNA, 98 nt, synthetic construct
  source 1..98: mol_type = other RNA
ctcagcagcagccacaactccctcgcgcaacttcaagcactttcatgactacatccacggctatcatactcctttacaccacactgtcgtcgaatggc

SEQ ID NO: 255 - RNA, 100 nt, synthetic construct
  source 1..100: mol_type = other RNA
ctcagcagcagccacaactccctcgcgcaacttattaagtcctttcatgaagccatctacggctatcatactcctttacaccacactgtcgtcgaatggc

SEQ ID NO: 256 - RNA, 99 nt, synthetic construct
  source 1..99: mol_type = other RNA
ctcagcagcagccacaactccctcgcgcaactttgaaagtcattcatgtatacaaccacggctatcatactcctttacaccacactgtcgtcgaatggc

SEQ ID NO: 257 - RNA, 99 nt, synthetic construct
  source 1..99: mol_type = other RNA
ctcagcagcagccacaactccctcctctcgagttgaaagtcctggcatgaatacaccacggctacggatagagcttacaccacactgtcgtcgaatggc

SEQ ID NO: 258 - RNA, 103 nt, synthetic construct
  source 1..103: mol_type = other RNA
ctcagcagcagccacaactccctcgcgcaactttaccaaaaggggtttcatctttacatccacggctatcatactcctttacaccacactgtcgtcgaatggc

SEQ ID NO: 259 - RNA, 101 nt, synthetic construct
  source 1..101: mol_type = other RNA
ctcagcagcagccacaactccctcgcgcaactttataaagtcctttggatgaatacaccacggctatcatactcctttacaccacactgtcgtcgaatggc

SEQ ID NO: 260 - RNA, 98 nt, synthetic construct
  source 1..98: mol_type = other RNA
ctcagcagcagccacaactccctcgcgcaactttgacagtattcatgtttacatccacggctatcatactcctttacaccacactgtcgtcgaatggc

SEQ ID NO: 261 - RNA, 99 nt, synthetic construct
  source 1..99: mol_type = other RNA
ctcagcagcagccacaactccctcgcgcaactttgaaagcaatttcatgcatagaccacggctacataattccttttggagtcactgtcgtcgaatggc

SEQ ID NO: 262 - RNA, 102 nt, synthetic construct
  source 1..102: mol_type = other RNA
ctcagcagcagccacaactccctcgcgcaacttttccaaagtcctttgatgaatacaagcacggctatcatactcctttacaccacactgtcgtcgaatggc

SEQ ID NO: 263 - RNA, 98 nt, synthetic construct
  source 1..98: mol_type = other RNA
ctcagcagcagccacaactccctcctgaatgattgaaagtcctttcatgaatacatccacggcatgatctgagttacaccacactgtcgtcgaatggc

SEQ ID NO: 264 - RNA, 97 nt, synthetic construct
  source 1..97: mol_type = other RNA
ctcagcagcagccacaactccctcgcgcaactttgaaatatttcatctctacaccacggctatcatactcctttacaccacactgtcgtcgaatggc

SEQ ID NO: 265 - RNA, 103 nt, synthetic construct
  source 1..103: mol_type = other RNA
ctcagcagcagccacaactccctcgcgcaactttgaaactcttcctttcatgctgcacatccacggcaatcgccgactttacaccacactgtcgtcgaatggc

SEQ ID NO: 266 - RNA, 98 nt, synthetic construct
  source 1..98: mol_type = other RNA
ctcagcagcagccacaactccctcgcgcaactttgaaagatttcatgaatagatctacggctatcatactcctttacaccacactgtcgtcgaatggc

SEQ ID NO: 267 - RNA, 98 nt, synthetic construct
  source 1..98: mol_type = other RNA
ctcagcagcagccacaactccctcctatagagttgtgtcctttcatgtatacatctacggctaatcgccgactttacaccacactgtcgtcgaatggc

SEQ ID NO: 268 - RNA, 100 nt, synthetic construct
  source 1..100: mol_type = other RNA
ctcagcagcagccacaactccctcgcgcaacttcaggaaagatttcatgaatacatctacggctatcatactcctttacaccacactgtcgtcgaatggc

SEQ ID NO: 269 - RNA, 99 nt, synthetic construct
  source 1..99: mol_type = other RNA
ctcagcagcagccacaactccctcgcgcaactttgaaagtgtttcctgaatactgccacggctatcatactcctttacaccacactgtcgtcgaatggc

SEQ ID NO: 270 - RNA, 99 nt, synthetic construct
  source 1..99: mol_type = other RNA
ctcagcagcagccacaactccctcgcgcaactttgaaagtggcttcatgcatagaccacggctacataattccttttggagtcactgtcgtcgaatggc

SEQ ID NO: 271 - RNA, 99 nt, synthetic construct
  source 1..99: mol_type = other RNA
ctcagcagcagccacaactccctcctatagagttgaaagactttcatgtctacatctacggctaatcgccgactttacaccacactgtcgtcgaatggc

SEQ ID NO: 272 - RNA, 100 nt, synthetic construct
  source 1..100: mol_type = other RNA
ctcagcagcagccacaactccctcctatagagttgaccctcctttcatgaataccgctacggctaatcgccgactttacaccacactgtcgtcgaatggc

SEQ ID NO: 273 - RNA, 99 nt, synthetic construct
  source 1..99: mol_type = other RNA
ctcagcagcagccacaactccctcgcgcaactttgaaagtcgttcatgacgccatctacggctatcatactcctttacaccacactgtcgtcgaatggc

SEQ ID NO: 274 - RNA, 100 nt, synthetic construct
  source 1..100: mol_type = other RNA
ctcagcagcagccacaactccctcctctcgagttgaaagtgacttcatgagtacatccacggctacggatagagcttacaccacactgtcgtcgaatggc

SEQ ID NO: 275 - RNA, 99 nt, synthetic construct
  source 1..99: mol_type = other RNA
ctcagcagcagccacaactccctcgcgcaactttgaaagtgttcatgaataaaatctacggctatcatactcctttacaccacactgtcgtcgaatggc

SEQ ID NO: 276 - RNA, 101 nt, synthetic construct
  source 1..101: mol_type = other RNA
ctcagcagcagccacaactccctcctctcgagttgaaagggacctttcatgaatacaccacggctacggatagagcttacaccacactgtcgtcgaatggc

SEQ ID NO: 277 - RNA, 102 nt, synthetic construct
  source 1..102: mol_type = other RNA
ctcagcagcagccacaactccctcgcgcaactttgaacgtgtcctttcatgaatacaagcacggctatcatactcctttacaccacactgtcgtcgaatggc

SEQ ID NO: 278 - RNA, 100 nt, synthetic construct
  source 1..100: mol_type = other RNA
ctcagcagcagccacaactccctcctctcgagttgaaagtagattcatgattacatccacggctacggatagagcttacaccacactgtcgtcgaatggc

SEQ ID NO: 279 - RNA, 103 nt, synthetic construct
  source 1..103: mol_type = other RNA
ctcagcagcagccacaactccctcgcgcaacttaacgatcagtcctttcatgaatacatgcacggctatcatactcctttacaccacactgtcgtcgaatggc

SEQ ID NO: 280 - RNA, 96 nt, synthetic construct
  source 1..96: mol_type = other RNA
ctcagcagcagccacaactccctcgcgcaactttgaaccctttcacatacatctacggctatcatactcctttacaccacactgtcgtcgaatggc

SEQ ID NO: 281 - RNA, 99 nt, synthetic construct
  source 1..99: mol_type = other RNA
ctcagcagcagccacaactccctcgcgcaactttgaaagtccggtcatgcatagaccacggctacataattccttttggagtcactgtcgtcgaatggc

SEQ ID NO: 282 - RNA, 100 nt, synthetic construct
  source 1..100: mol_type = other RNA
ctcagcagcagccacaactccctcctgaatgattgaaagtccttccctgaatacatccacggcgtcatcattcctttacaccacactgtcgtcgaatggc

SEQ ID NO: 283 - RNA, 98 nt, synthetic construct
  source 1..98: mol_type = other RNA
ctcagcagcagccacaactccctcgcgcaactttgaaagtgttcatgcctaccgccacggctatcatactcctttacaccacactgtcgtcgaatggc

SEQ ID NO: 284 - RNA, 99 nt, synthetic construct
  source 1..99: mol_type = other RNA
ctcagcagcagccacaactccctcctctcgagttgaatcccctttcatgaatacaccacggctacggatagagcttacaccacactgtcgtcgaatggc

SEQ ID NO: 285 - RNA, 102 nt, synthetic construct
  source 1..102: mol_type = other RNA
ctcagcagcagccacaactccctcctctcgagttgaaagaaacctttcatgaataccaccacggctacggatagagcttacaccacactgtcgtcgaatggc

SEQ ID NO: 286 - DNA, 95 bp, synthetic construct
  source 1..95: mol_type = other DNA
cagcagccacaactccctcctatagagttgaaagtcctttcatgaatacatctacggctaatcgccgactttacaccacactgtcgtcgaatggc

SEQ ID NO: 287 - DNA, 85 bp, synthetic construct
  source 1..85: mol_type = other DNA
agcagccacaactccctcctacaaacttgaaagtcctttcatgaatacaaccacggctaattgacggagattacaccacactgtc

SEQ ID NO: 288 - DNA, 95 bp, synthetic construct
  source 1..95: mol_type = other DNA
cagcagccacaactcccaggatcgcctttgaaagtccttccctgaatacatccacggctacgacagtcctttacaccacactgtcgtcgaatggc

SEQ ID NO: 289 - DNA, 85 bp, synthetic construct
  source 1..85: mol_type = other DNA
gccacaactccctccttgataggcgaaagtcctttcatgaataactccacggctatcatactcctttacaccacactgtcgtcga

SEQ ID NO: 290 - DNA, 90 bp, synthetic construct
  source 1..90: mol_type = other DNA
cagcagccacaactccctcgcgcaactttgaaagtcctttcatgcatagatccacggctacataattccttttggagtcactgtcgtcga

SEQ ID NO: 291 - DNA, 100 bp, synthetic construct
  source 1..100: mol_type = other DNA
ctcagcagcagccacaactccaatgaaggcctttgaaagtcctttcatgaatacatctacggctacatatctcctttacaccacactgtcgtcgaatggc

SEQ ID NO: 292 - DNA, 100 bp, synthetic construct
  source 1..100: mol_type = other DNA
ctcagcagcagccacaactccagaataggcctttgaaagtcctttcatgaataactccacggctataaatctcctttacaccacactgtcgtcgaatggc

SEQ ID NO: 293 - RNA, 95 nt, synthetic construct
  source 1..95: mol_type = other RNA
cagcagccacaactccctcctatagagttgaaagtcctttcatgaatacatctacggctaatcgccgactttacaccacactgtcgtcgaatggc

SEQ ID NO: 294 - RNA, 85 nt, synthetic construct
  source 1..85: mol_type = other RNA
agcagccacaactccctcctacaaacttgaaagtcctttcatgaatacaaccacggctaattgacggagattacaccacactgtc

SEQ ID NO: 295 - RNA, 95 nt, synthetic construct
  source 1..95: mol_type = other RNA
cagcagccacaactcccaggatcgcctttgaaagtccttccctgaatacatccacggctacgacagtcctttacaccacactgtcgtcgaatggc

SEQ ID NO: 296 - RNA, 85 nt, synthetic construct
  source 1..85: mol_type = other RNA
gccacaactccctccttgataggcgaaagtcctttcatgaataactccacggctatcatactcctttacaccacactgtcgtcga

SEQ ID NO: 297 - RNA, 90 nt, synthetic construct
  source 1..90: mol_type = other RNA
cagcagccacaactccctcgcgcaactttgaaagtcctttcatgcatagatccacggctacataattccttttggagtcactgtcgtcga

SEQ ID NO: 298 - RNA, 100 nt, synthetic construct
  source 1..100: mol_type = other RNA
ctcagcagcagccacaactccaatgaaggcctttgaaagtcctttcatgaatacatctacggctacatatctcctttacaccacactgtcgtcgaatggc

SEQ ID NO: 299 - RNA, 100 nt, synthetic construct
  source 1..100: mol_type = other RNA
ctcagcagcagccacaactccagaataggcctttgaaagtcctttcatgaataactccacggctataaatctcctttacaccacactgtcgtcgaatggc

SEQ ID NO: 300 - RNA, 100 nt, unidentified
  source 1..100: mol_type = unassigned RNA
gccattcgacgacagtgtggtgtaaaggaattcattagccatggatgtattcatgaaaggactttcaaaggccaaggagggagttgtggctgctgctgag

SEQ ID NO: 301 - DNA, 32 bp, Homo sapiens
  source 1..32: mol_type = genomic DNA
taggctttctggctttttaccggaaagcccct

SEQ ID NO: 302 - RNA, 32 nt, Homo sapiens
  source 1..32: mol_type = genomic RNA
taggctttctggctttttaccggaaagcccct

SEQ ID NO: 303 - DNA, 31 bp, Mus sp.
  source 1..31: mol_type = genomic DNA
caggttttctgacttcggtcggaaaacccct

SEQ ID NO: 304 - RNA, 31 nt, Mus sp.
  source 1..31: mol_type = genomic RNA
caggttttctgacttcggtcggaaaacccct

SEQ ID NO: 305 - DNA, 11 bp, unidentified
  source 1..11: mol_type = unassigned DNA
aatttttggag

SEQ ID NO: 306 - RNA, 11 nt, unidentified
  source 1..11: mol_type = unassigned RNA
aatttttggag

SEQ ID NO: 307 - DNA, 249 bp, Homo sapiens
  source 1..249: mol_type = genomic DNA
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 308 - DNA, 341 bp, Mus sp.
  source 1..341: mol_type = genomic DNA
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 309 - DNA, 263 bp, Homo sapiens
  source 1..263: mol_type = genomic DNA
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 310 - DNA, 257 bp, Mus sp.
  source 1..257: mol_type = genomic DNA
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 311 - DNA, 393 bp, Homo sapiens
  source 1..393: mol_type = genomic DNA
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 312 - DNA, 698 bp, Cytomegalovirus sp.
  source 1..698: mol_type = genomic DNA
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 313 - DNA, 56 bp, synthetic construct
  source 1..56: mol_type = other DNA
gccatcagtcgccggtcccaagcccggataaaatgggagggggcgggaaaccgcct

SEQ ID NO: 314 - RNA, 56 nt, synthetic construct
  source 1..56: mol_type = other RNA
gccatcagtcgccggtcccaagcccggataaaatgggagggggcgggaaaccgcct

SEQ ID NO: 315-316 - skipped (2 entries)

SEQ ID NO: 317 - DNA, 55 bp, synthetic construct
  source 1..55: mol_type = other DNA
aacactgccaatgccggtcccaagcccggataaaagtggagggtacagtccacgc

SEQ ID NO: 318 - RNA, 55 nt, synthetic construct
  source 1..55: mol_type = other RNA
aacactgccaatgccggtcccaagcccggataaaagtggagggtacagtccacgc

SEQ ID NO: 319 - skipped

SEQ ID NO: 320 - DNA, 21 bp, synthetic construct
  source 1..21: mol_type = other DNA
aaccatgccgactgatggcag

SEQ ID NO: 321 - DNA, 26 bp, synthetic construct
  source 1..26: mol_type = other DNA
gatgtcaggtgcggctgactaccgtc

SEQ ID NO: 322 - RNA, 21 nt, synthetic construct
  source 1..21: mol_type = other RNA
aaccatgccgactgatggcag

SEQ ID NO: 323 - RNA, 26 nt, synthetic construct
  source 1..26: mol_type = other RNA
gatgtcaggtgcggctgactaccgtc

SEQ ID NO: 324 - skipped

SEQ ID NO: 325 - DNA, 95 bp, synthetic construct
  source 1..95: mol_type = other DNA
cagcagccacaactcccgaaggagcctttgaaagtccttaattgaatacatccacggctatacttatcctttacaccacactgtcgtcgaatggc

SEQ ID NO: 326 - DNA, 95 bp, synthetic construct
  source 1..95: mol_type = other DNA
cagcagccacaactcccgaaggagcctttgaaagtccttaattgaatacatccacggctatacttatcctttacaccacactgtcgttgagtggt

SEQ ID NO: 327 - DNA, 95 bp, synthetic construct
  source 1..95: mol_type = other DNA
cagcagccacaactcccgaaggagcctttgaaagtccttaattgaatacatccacggctatacttatcctttacaccacgctgttgttgagtggt

SEQ ID NO: 328 - DNA, 95 bp, synthetic construct
  source 1..95: mol_type = other DNA
cagcagccacaactcccgaaggagcctttgaaagtccttaattgaatacatccacggttatacttattctttgcatcacgctgttgttgagtggt

SEQ ID NO: 329 - DNA, 100 bp, synthetic construct
  source 1..100: mol_type = other DNA
ctcagcagcagccacaactccgtgcggggcctttgaaagtcctttcatgaatacatctacggctatactggtcctttacaccacactgttgtcgagtggt

SEQ ID NO: 330 - DNA, 100 bp, synthetic construct
  source 1..100: mol_type = other DNA
ctcagcagcagccacaactccgtgcggggcctttgaaagtcctttcatgaatacatctacggctatactggtcctttacaccgcgctgttgtcgagtggt

SEQ ID NO: 331 - DNA, 100 bp, synthetic construct
  source 1..100: mol_type = other DNA
ctcagcagcagccacaactccgtgcggggcctttgaaagtcctttcatgaatacatctacggctatactggtcctttgcatcgcgctgttgtcgagtggt

SEQ ID NO: 332 - DNA, 100 bp, synthetic construct
  source 1..100: mol_type = other DNA
ctcagcagcagccacaactccgtgcggggcctttgaaagtcctttcatgaatacgtctacggctatactggttctttgcatcgcgctgttgtcgagtggt

SEQ ID NO: 333 - RNA, 95 nt, synthetic construct
  source 1..95: mol_type = other RNA
cagcagccacaactcccgaaggagcctttgaaagtccttaattgaatacatccacggctatacttatcctttacaccacactgtcgtcgaatggc

SEQ ID NO: 334 - RNA, 95 nt, synthetic construct
  source 1..95: mol_type = other RNA
cagcagccacaactcccgaaggagcctttgaaagtccttaattgaatacatccacggctatacttatcctttacaccacactgtcgttgagtggt

SEQ ID NO: 335 - RNA, 95 nt, synthetic construct
  source 1..95: mol_type = other RNA
cagcagccacaactcccgaaggagcctttgaaagtccttaattgaatacatccacggctatacttatcctttacaccacgctgttgttgagtggt

SEQ ID NO: 336 - RNA, 95 nt, synthetic construct
  source 1..95: mol_type = other RNA
cagcagccacaactcccgaaggagcctttgaaagtccttaattgaatacatccacggttatacttattctttgcatcacgctgttgttgagtggt

SEQ ID NO: 337 - RNA, 100 nt, synthetic construct
  source 1..100: mol_type = other RNA
ctcagcagcagccacaactccgtgcggggcctttgaaagtcctttcatgaatacatctacggctatactggtcctttacaccacactgttgtcgagtggt

SEQ ID NO: 338 - RNA, 100 nt, synthetic construct
  source 1..100: mol_type = other RNA
ctcagcagcagccacaactccgtgcggggcctttgaaagtcctttcatgaatacatctacggctatactggtcctttacaccgcgctgttgtcgagtggt

SEQ ID NO: 339 - RNA, 100 nt, synthetic construct
  source 1..100: mol_type = other RNA
ctcagcagcagccacaactccgtgcggggcctttgaaagtcctttcatgaatacatctacggctatactggtcctttgcatcgcgctgttgtcgagtggt

SEQ ID NO: 340 - RNA, 100 nt, synthetic construct
  source 1..100: mol_type = other RNA
ctcagcagcagccacaactccgtgcggggcctttgaaagtcctttcatgaatacgtctacggctatactggttctttgcatcgcgctgttgtcgagtggt

SEQ ID NO: 341 - RNA, 100 nt, synthetic construct
  source 1..100: mol_type = other RNA
gccacaactccctcgcgcaactttgaaagtcctttcatgcatagatccacggctccaaaactccttttggagtcactgtcgtcgaatggccactcccagt

SEQ ID NO: 342 - RNA, 99 nt, synthetic construct
  source 1..99: mol_type = other RNA
gccacaactccctcgcgcaactttgaaagtcctttcatgcatagatccacggctccaaattccttttggagtcactgtcgtcgaatggccactcccagt

SEQ ID NO: 343 - RNA, 100 nt, synthetic construct
  source 1..100: mol_type = other RNA
gccacaactccctcgcgcaactttgaaagtcctttcatgcatagatccacggctccataattccttttggagtcactgtcgtcgaatggccactcccagt

SEQ ID NO: 344 - RNA, 100 nt, synthetic construct
  source 1..100: mol_type = other RNA
gccacaactccctcgcgcaactttgaaagtcctttcatgcatagatccacggctgcataattccttttggagtcactgtcgtcgaatggccactcccagt

SEQ ID NO: 345 - RNA, 100 nt, synthetic construct
  source 1..100: mol_type = other RNA
gccacaactccctcctacaaacttgaaagtcctttcatgaatacaaccacggctattgacggagattacaccacactgtcgtcgaatggccactcccagt

SEQ ID NO: 346 - RNA, 101 nt, synthetic construct
  source 1..101: mol_type = other RNA
gccacaactccctcctacaaacttgaaagtcctttcatgaatacaaccacggctatttgacggagattacaccacactgtcgtcgaatggccactcccagt

SEQ ID NO: 347 - RNA, 99 nt, synthetic construct
  source 1..99: mol_type = other RNA
gccacaactccctcctacaaacttgaaagtcctttcatgaatacaaccacggctttgacggagattacaccacactgtcgtcgaatggccactcccagt

SEQ ID NO: 348 - RNA, 99 nt, synthetic construct
  source 1..99: mol_type = other RNA
gccacaactccctcctatagagttgaaagtcctttcatgaatacatctacggctatcgccgactttacaccacactgtcgtcgaatggccactcccagt

SEQ ID NO: 349 - RNA, 99 nt, synthetic construct
  source 1..99: mol_type = other RNA
gccacaactccctcctatagagttgaaagtcctttcatgaatacatctacggctctcgccgactttacaccacactgtcgtcgaatggccactcccagt

SEQ ID NO: 350 - RNA, 99 nt, synthetic construct
  source 1..99: mol_type = other RNA
gccacaactccctcctatagcgttgaaagtcctttcatgaatacatctacggctctcgccgactttacaccacactgtcgtcgaatggccactcccagt

SEQ ID NO: 351 - RNA, 98 nt, synthetic construct
  source 1..98: mol_type = other RNA
gccacaactccctccttgacggcggaaagtcctttcatggcacatccacggctaccaattcctaccgtgcacactgtcgtcgaatggccactcccagt

SEQ ID NO: 352 - RNA, 98 nt, synthetic construct
  source 1..98: mol_type = other RNA
gccacaactccctccttgatggcggaaagtcctttcatggcacatccacggctaccaattcctaccgtgcacactgtcgtcgaatggccactcccagt

SEQ ID NO: 353 - RNA, 98 nt, synthetic construct
  source 1..98: mol_type = other RNA
gccacaactccctccttgatggcggaaagtcctttcatggcacatccacggctatcaattcctaccgtgcacactgtcgtcgaatggccactcccagt

SEQ ID NO: 354 - RNA, 100 nt, synthetic construct
  source 1..100: mol_type = other RNA
gccacaactccctccttgtaggaggaaagtcctttcattaatacatccacggcaatggaattcctcctctacacactgtcgtcgaatggccactcccagt

SEQ ID NO: 355 - RNA, 101 nt, synthetic construct
  source 1..101: mol_type = other RNA
gccacaactccctccttgtaggaggaaagtcctttcattaatacatccacggcacttggaattcctcctctacacactgtcgtcgaatggccactcccagt

SEQ ID NO: 356 - RNA, 100 nt, synthetic construct
  source 1..100: mol_type = other RNA
gccacaactccctccttgtaggaggaaagtcctttcattaatacatccacggctatggaattcctcctctacacactgtcgtcgaatggccactcccagt

SEQ ID NO: 357 - RNA, 101 nt, synthetic construct
  source 1..101: mol_type = other RNA
gccacaactccctccttgtaggaggaaagtcctttcattaatacatccacggctcttggaattcctcctctacacactgtcgtcgaatggccactcccagt

SEQ ID NO: 358 - DNA, 100 bp, synthetic construct
  source 1..100: mol_type = other DNA
gccacaactccctcgcgcaactttgaaagtcctttcatgcatagatccacggctccaaaactccttttggagtcactgtcgtcgaatggccactcccagt

SEQ ID NO: 359 - DNA, 99 bp, synthetic construct
  source 1..99: mol_type = other DNA
gccacaactccctcgcgcaactttgaaagtcctttcatgcatagatccacggctccaaattccttttggagtcactgtcgtcgaatggccactcccagt

SEQ ID NO: 360 - DNA, 100 bp, synthetic construct
  source 1..100: mol_type = other DNA
gccacaactccctcgcgcaactttgaaagtcctttcatgcatagatccacggctccataattccttttggagtcactgtcgtcgaatggccactcccagt

SEQ ID NO: 361 - DNA, 100 bp, synthetic construct
  source 1..100: mol_type = other DNA
gccacaactccctcgcgcaactttgaaagtcctttcatgcatagatccacggctgcataattccttttggagtcactgtcgtcgaatggccactcccagt

SEQ ID NO: 362 - DNA, 100 bp, synthetic construct
  source 1..100: mol_type = other DNA
gccacaactccctcctacaaacttgaaagtcctttcatgaatacaaccacggctattgacggagattacaccacactgtcgtcgaatggccactcccagt

SEQ ID NO: 363 - DNA, 101 bp, synthetic construct
  source 1..101: mol_type = other DNA
gccacaactccctcctacaaacttgaaagtcctttcatgaatacaaccacggctatttgacggagattacaccacactgtcgtcgaatggccactcccagt

SEQ ID NO: 364 - DNA, 99 bp, synthetic construct
  source 1..99: mol_type = other DNA
gccacaactccctcctacaaacttgaaagtcctttcatgaatacaaccacggctttgacggagattacaccacactgtcgtcgaatggccactcccagt

SEQ ID NO: 365 - DNA, 99 bp, synthetic construct
  source 1..99: mol_type = other DNA
gccacaactccctcctatagagttgaaagtcctttcatgaatacatctacggctatcgccgactttacaccacactgtcgtcgaatggccactcccagt

SEQ ID NO: 366 - DNA, 99 bp, synthetic construct
  source 1..99: mol_type = other DNA
gccacaactccctcctatagagttgaaagtcctttcatgaatacatctacggctctcgccgactttacaccacactgtcgtcgaatggccactcccagt

SEQ ID NO: 367 - DNA, 99 bp, synthetic construct
  source 1..99: mol_type = other DNA
gccacaactccctcctatagcgttgaaagtcctttcatgaatacatctacggctctcgccgactttacaccacactgtcgtcgaatggccactcccagt

SEQ ID NO: 368 - DNA, 98 bp, synthetic construct
  source 1..98: mol_type = other DNA
gccacaactccctccttgacggcggaaagtcctttcatggcacatccacggctaccaattcctaccgtgcacactgtcgtcgaatggccactcccagt

SEQ ID NO: 369 - DNA, 98 bp, synthetic construct
  source 1..98: mol_type = other DNA
gccacaactccctccttgatggcggaaagtcctttcatggcacatccacggctaccaattcctaccgtgcacactgtcgtcgaatggccactcccagt

SEQ ID NO: 370 - DNA, 98 bp, synthetic construct
  source 1..98: mol_type = other DNA
gccacaactccctccttgatggcggaaagtcctttcatggcacatccacggctatcaattcctaccgtgcacactgtcgtcgaatggccactcccagt

SEQ ID NO: 371 - DNA, 100 bp, synthetic construct
  source 1..100: mol_type = other DNA
gccacaactccctccttgtaggaggaaagtcctttcattaatacatccacggcaatggaattcctcctctacacactgtcgtcgaatggccactcccagt

SEQ ID NO: 372 - DNA, 101 bp, synthetic construct
  source 1..101: mol_type = other DNA
gccacaactccctccttgtaggaggaaagtcctttcattaatacatccacggcacttggaattcctcctctacacactgtcgtcgaatggccactcccagt

SEQ ID NO: 373 - DNA, 100 bp, synthetic construct
  source 1..100: mol_type = other DNA
gccacaactccctccttgtaggaggaaagtcctttcattaatacatccacggctatggaattcctcctctacacactgtcgtcgaatggccactcccagt

SEQ ID NO: 374 - DNA, 101 bp, synthetic construct
  source 1..101: mol_type = other DNA
gccacaactccctccttgtaggaggaaagtcctttcattaatacatccacggctcttggaattcctcctctacacactgtcgtcgaatggccactcccagt

SEQ ID NO: 375 - RNA, 100 nt, synthetic construct
  source 1..100: mol_type = other RNA
ctcagcagcagccacaactccctcgcgcaactttgaaagtcctttcatgcatagatccacggctacataattccttttggagtcactgtcgtcgaatggc

SEQ ID NO: 376 - RNA, 100 nt, synthetic construct
  source 1..100: mol_type = other RNA
ctcagcagcagccacaactccctcctatagagttgaaagtcctttcatgaatacatctacggctaatcgccgactttacaccacactgtcgtcgaatggc

SEQ ID NO: 377 - DNA, 98 bp, synthetic construct
  source 1..98: mol_type = other DNA
ctcagcagcagccacaactccctcctgaatgattgaaagtcctttcatgaatacatccacggcatgatctgagttacaccacactgtcgtcgaatggc

SEQ ID NO: 378 - DNA, 100 bp, synthetic construct
  source 1..100: mol_type = other DNA
ctcagcagcagccacaactccctccttgtaaggagaaagtcctttcatgaatacatccacggcggctgaattcgcgctgaccacactgtcgtcgaatggc

SEQ ID NO: 379 - DNA, 100 bp, synthetic construct
  source 1..100: mol_type = other DNA
ctcagcagcagccacaactccctcctctcgagttgaaagtcctttcatgaatacatccacggctacggatagagcttacaccacactgtcgtcgaatggc

SEQ ID NO: 380 - RNA, 100 nt, synthetic construct
  source 1..100: mol_type = other RNA
ctcagcagcagccacaactccctccttgtaaggagaaagtcctttcatgaatacatccacggcggctgaattcgcgctgaccacactgtcgtcgaatggc

SEQ ID NO: 381 - RNA, 100 nt, synthetic construct
  source 1..100: mol_type = other RNA
ctcagcagcagccacaactccctcctctcgagttgaaagtcctttcatgaatacatccacggctacggatagagcttacaccacactgtcgtcgaatggc

SEQ ID NO: 382-389 - skipped (8 entries)

SEQ ID NO: 390 - RNA, 95 nt, synthetic construct
  source 1..95: mol_type = other RNA
cagcagccacaactcccgaaggagcctttgaaagtccttaattgaatacatccacggctatacttatcctttacaccacactgtcgtcgaatggt

SEQ ID NO: 391 - RNA, 95 nt, synthetic construct
  source 1..95: mol_type = other RNA
cagcagccacaactcccgaaggagcctttgaaagtccttaattgaatacatccacggctatacttatcctttgcatcacgctgttgttgagtggt

SEQ ID NO: 392 - RNA, 95 nt, synthetic construct
  source 1..95: mol_type = other RNA
cagcagccacaactcccgaaggagcctttgaaagtccttaattgagtatatccacggttatacttattctttgcatcacgctgttgttgagtggt

SEQ ID NO: 393 - RNA, 95 nt, synthetic construct
  source 1..95: mol_type = other RNA
cagcagccacaactctcgaaggagcctttgagagtccttaattgagtatatccacggttatacttattctttgcatcacgctgttgttgagtggt

SEQ ID NO: 394 - RNA, 95 nt, synthetic construct
  source 1..95: mol_type = other RNA
cagtagccacgactctcgaaggagcctttgagagtccttaattgagtatatccacggttatacttattctttgcatcacgctgttgttgagtggt

SEQ ID NO: 395 - RNA, 95 nt, synthetic construct
  source 1..95: mol_type = other RNA
cagcagccacaactcccgaaggagcctttgaaagtcctttattgaatacatccacggctaatgaaatcctttacaccacactgtcgtcgaatggc

SEQ ID NO: 396 - RNA, 100 nt, synthetic construct
  source 1..100: mol_type = other RNA
ctcagcagcagccacaactccgtggtaggcctttgaaagtcctttcatgaatacatctacggcattccttctcctttacaccacactgtcgtcgaatggc

SEQ ID NO: 397 - RNA, 100 nt, synthetic construct
  source 1..100: mol_type = other RNA
ctcagcagcggccacagctccatggaaggcttttggaagttcttttatgagtacgtctacggcatcactcctcctttgcatcacactgtcgttgagtggc

SEQ ID NO: 398 - RNA, 100 nt, synthetic construct
  source 1..100: mol_type = other RNA
ctcagcagcggccacagctccgtggaaggcttttggaagttcttttatgagtacgtctacggcatccctcctcctttgcatcacactgtcgttgagtggc

SEQ ID NO: 399 - RNA, 100 nt, synthetic construct
  source 1..100: mol_type = other RNA
ctcagtagcggccacagctccgtggaaggcttttggaagttcttttatgagtacgtctacggcatccctcctcctttgcatcacactgtcgttgagtggc

SEQ ID NO: 400 - RNA, 100 nt, synthetic construct
  source 1..100: mol_type = other RNA
ctcagtagcggccacagctccgtggaaggcttttggaagttcttttatgagtacgtctacggcattcctcctcctttgcatcacactgtcgttgagtggc

SEQ ID NO: 401 - RNA, 100 nt, synthetic construct
  source 1..100: mol_type = other RNA
ctcagtagcggccacagctccgtggaaggcttttggaagttcttttatgagtacgtctacggcattcctcctcctttgcatcgcactgtcgttgagtggc

SEQ ID NO: 402 - RNA, 100 nt, synthetic construct
  source 1..100: mol_type = other RNA
ctcagtagcggccacagctccgtggaaggcttttggaagttcttttatgagtacgtctacggcattccttctcctttgcatcgcactgtcgttgagtggc

SEQ ID NO: 403 - RNA, 100 nt, synthetic construct
  source 1..100: mol_type = other RNA
ctcagtagcggccacagctccgtggtaggcttttggaagttcttttatgagtacgtctacggcattccttctcctttgcatcgcactgtcgttgagtggc

SEQ ID NO: 404 - RNA, 100 nt, synthetic construct
  source 1..100: mol_type = other RNA
cttagtagcggccacagctccgtggtaggcttttggaagttcttttatgagtacgtctacggcattccttctcctttgcatcgcactgtcgttgagtggc

SEQ ID NO: 405 - RNA, 100 nt, synthetic construct
  source 1..100: mol_type = other RNA
ctcagcagcagccacaactccgtggacggcctttgaaagtcctttcatgaatagttccacggctacggtcatcctttacaccacactgtcgtcgaatggc

SEQ ID NO: 406 - RNA, 100 nt, synthetic construct
  source 1..100: mol_type = other RNA
ctcagcagcagtcacgactccgtggtaggcctttgagagtcctttcgtgaatagttccacggctacggtcatcctttacatcacactgtcgtcggatggc

SEQ ID NO: 407 - RNA, 100 nt, synthetic construct
  source 1..100: mol_type = other RNA
ctcagcagcagtcacgactccgtggacggcctttgagagtcctttcgtgaatagttccacggctacggtcatcctttacatcacactgtcgtcggatggc

SEQ ID NO: 408 - RNA, 100 nt, synthetic construct
  source 1..100: mol_type = other RNA
ctcagcagcagtcacaactccgtggacggcctttgagagttctttcgtgaatagttccacggctacggtcatcctttacatcacactgttgtcggatggc

SEQ ID NO: 409 - RNA, 100 nt, synthetic construct
  source 1..100: mol_type = other RNA
ctcagcagcagtcacgactccgtggacggcctttgagagttctttcgtgaatagttccacggctacggtcatcctttacatcacactgttgtcggatggc

SEQ ID NO: 410 - RNA, 100 nt, synthetic construct
  source 1..100: mol_type = other RNA
ctcagcagcagtcacgactccgtggacggtctttgagagtcctttcgtgaatagttccacggctacggtcatcctttacatcacgctgttgtcggatggc

SEQ ID NO: 411 - RNA, 100 nt, synthetic construct
  source 1..100: mol_type = other RNA
ctcagcagcagtcacgactccgtggacggtctttgagagttctttcgtgaatagttccacggctacggtcatcctttacatcacgctgttgtcggatggc

SEQ ID NO: 412 - RNA, 100 nt, synthetic construct
  source 1..100: mol_type = other RNA
ctcagcggcagtcacgactccgtggacggtctttgagagtcctttcgtgaatagttccacggctacggtcatcctttacatcacgctgttgtcggatggt

SEQ ID NO: 413 - RNA, 100 nt, synthetic construct
  source 1..100: mol_type = other RNA
ctcagcggcagtcacgactccgtggacggtctttgagagttctttcgtgaatagttccacggctacggtcatcctttacatcacgctgttgtcggatggt

SEQ ID NO: 414 - RNA, 90 nt, synthetic construct
  source 1..90: mol_type = other RNA
cagcagccacaactccctcaaaccgctttgaaagtcctttcatgtatagatccacggctatacaattccttttgtggtcgctgttgttgg

SEQ ID NO: 415 - RNA, 90 nt, synthetic construct
  source 1..90: mol_type = other RNA
cagcagccacagctctctcacaccgctttggaagttctttcgtgcatggatccacggctatacaatttcttttgtggtcgctgttgttga

SEQ ID NO: 416 - RNA, 90 nt, synthetic construct
  source 1..90: mol_type = other RNA
cagcagccacagctctctcaaaccgctttggaagttctttcgtgcatggatccacggctatacaatttcttttgtggtcgctgttgttga

SEQ ID NO: 417 - RNA, 90 nt, synthetic construct
  source 1..90: mol_type = other RNA
cagcagccacagctctctcaaaccgctttggaggttctttcgtgcatggatccacggctatacaatttcttttgtggtcgctgttgttga

SEQ ID NO: 418 - RNA, 90 nt, synthetic construct
  source 1..90: mol_type = other RNA
cagtagccacagctctctcaaaccgctttggaggttctttcgtgcatggatccacggctatacaatttcttttgtggtcgctgttgttga

SEQ ID NO: 419 - RNA, 90 nt, synthetic construct
  source 1..90: mol_type = other RNA
cagtagccacagctctctcaaaccgctttggaggttctttcgtgcatggatccacggttatacaatttcttttgtggtcgctgttgttga

SEQ ID NO: 420 - RNA, 90 nt, synthetic construct
  source 1..90: mol_type = other RNA
cagtagccacagctctctcaaaccgctttggaggttctttcgtgcatggattcacggttatacaatttcttttgtggtcgctgttgttga

SEQ ID NO: 421 - RNA, 90 nt, synthetic construct
  source 1..90: mol_type = other RNA
cagtagccacagctctctcaaaccgctttggaggttctttcgtgtatggattcacggttatacaatttcttttgtggtcgctgttgttga

SEQ ID NO: 422 - RNA, 90 nt, synthetic construct
  source 1..90: mol_type = other RNA
cagtagccacagctctctcaaaccgctttggaggttctttcgtgtatggattcacggttatacaatttcttttgtggtcgctgttgttgg

SEQ ID NO: 423 - RNA, 90 nt, synthetic construct
  source 1..90: mol_type = other RNA
cagtagctacagctctctcaaaccgctttggaggttctttcgtgtatggattcacggttatacaatttcttttgtggtcgctgttgttgg

SEQ ID NO: 424 - RNA, 86 nt, synthetic construct
  source 1..86: mol_type = other RNA
cagcagccacaactccctcctacaaacttgaaagtcctttcatgaatacaaccacggctaattgacggagattacaccacactgtt

SEQ ID NO: 425 - RNA, 86 nt, synthetic construct
  source 1..86: mol_type = other RNA
cagcagccacgactcccttctccaagattgagagttcttttatgagtacacccacggctagttgatgaggattgcgccacgctgtt

SEQ ID NO: 426 - RNA, 86 nt, synthetic construct
  source 1..86: mol_type = other RNA
cagcagccacgactcccttctccaagattgagagttcttttatgagtacacccacggctagttgatgaggattgcgctacgctgtt

SEQ ID NO: 427 - RNA, 86 nt, synthetic construct
  source 1..86: mol_type = other RNA
cagcagtcacgactcccttctccaagattgagagttcttttatgagtacacccacggctagttgatgaggattgcgctacgctgtt

SEQ ID NO: 428 - RNA, 86 nt, synthetic construct
  source 1..86: mol_type = other RNA
cagcagtcacgactcccttctccaagattgagagttcttttataagtacacccacggctagttgatgaggattgcgctacgctgtt

SEQ ID NO: 429 - RNA, 86 nt, synthetic construct
  source 1..86: mol_type = other RNA
cagcagtcacgactctcttctccaagattgagagttcttttataagtacacccacggctagttgatgaggattgcgctacgctgtt

SEQ ID NO: 430 - RNA, 86 nt, synthetic construct
  source 1..86: mol_type = other RNA
cagcagtcacgactctcttctccaagattgagagttcttttataagtacacccacggctagttgataaggattgcgctacgctgtt

SEQ ID NO: 431 - RNA, 86 nt, synthetic construct
  source 1..86: mol_type = other RNA
cagtagtcacgactctcttctccaagattgagagttcttttataagtacacccacggctagttgataaggattgcgctacgctgtt

SEQ ID NO: 432 - RNA, 98 nt, synthetic construct
  source 1..98: mol_type = other RNA
ctcagcggcagtcacagctccttcctacatgattggaagtctttttatggatacgtccacggcatgatatgggttgcatcacgctgttgtcggatggc

SEQ ID NO: 433 - RNA, 98 nt, synthetic construct
  source 1..98: mol_type = other RNA
ctcagcggcagtcacagctccttcctacatgattggaagtctttttatggatacgtccacggcatgatatgggttgcatcacgctgttgtcggatggt

SEQ ID NO: 434 - RNA, 98 nt, synthetic construct
  source 1..98: mol_type = other RNA
ctcagcggcagtcacagctccttcctacatgattggaagtctttttatggatacgtccacggcatgatatggattgcatcacgctgttgtcggatggt

SEQ ID NO: 435 - RNA, 98 nt, synthetic construct
  source 1..98: mol_type = other RNA
ctcagcggcagtcacagctccttcctacatgattggaagtctttttatggatacgtccacggcatgatatggattgcatcacgctgttgttggatggt

SEQ ID NO: 436 - RNA, 98 nt, synthetic construct
  source 1..98: mol_type = other RNA
ctcagcggcagtcacagctccttcctacatgattggaagtctttttatggatacgtccacggcatgatatggattgcatcgcgctgttgttggatggt

SEQ ID NO: 437 - RNA, 98 nt, synthetic construct
  source 1..98: mol_type = other RNA
ctcagcggcagtcacagctccttcctacatgattggaagtctttttatggatacgtccacggcatgataaggattgcatcgcgctgttgttggatggt

SEQ ID NO: 438 - RNA, 98 nt, synthetic construct
  source 1..98: mol_type = other RNA
ctcagcggcagtcacagctccttcctacatgattggaggtctttttatggatacgtccacggcatgataaggattgcatcgcgctgttgttggatggt

SEQ ID NO: 439 - RNA, 98 nt, synthetic construct
  source 1..98: mol_type = other RNA
cttagcggcagtcacagctccttcctacatgattggaggtctttttatggatacgtccacggcatgataaggattgcatcgcgctgttgttggatggt

SEQ ID NO: 440 - RNA, 98 nt, synthetic construct
  source 1..98: mol_type = other RNA
cttagcggcagtcacagctccttcctacatgattggaggtctttttatggatacgtccacggcatgataaggattgcatcgcgctgttgttgggtggt

SEQ ID NO: 441 - DNA, 101 bp, synthetic construct
  source 1..101: mol_type = other DNA
ctcagcagcagccacaactccctcgcgcaacttaggaaagtgggttcatgcatagatccacggctacataattccttttggagtcactgtcgtcgaatggc

SEQ ID NO: 442 - DNA, 99 bp, synthetic construct
  source 1..99: mol_type = other DNA
ctcagcagcagccacaactccctcgcgcaactgggaaaggctttcatgcatagatccacggctacataattccttttggagtcactgtcgtcgaatggc

SEQ ID NO: 443 - DNA, 98 bp, synthetic construct
  source 1..98: mol_type = other DNA
ctcagcagcagccacaactccctcgcgcaacttcaagtggtttcatgcatagatccacggctacataattccttttggagtcactgtcgtcgaatggc

SEQ ID NO: 444 - DNA, 100 bp, synthetic construct
  source 1..100: mol_type = other DNA
ctcagcagcagccacaactccctcgcgcaacgaagaacaacctttcatgcatagatccacggctacataattccttttggagtcactgtcgtcgaatggc

SEQ ID NO: 445 - DNA, 101 bp, synthetic construct
  source 1..101: mol_type = other DNA
ctcagcagcagccacaactccctcgcgcaacttccgaaaggactttcatgcatagatccacggctacataattccttttggagtcactgtcgtcgaatggc

SEQ ID NO: 446 - DNA, 101 bp, synthetic construct
  source 1..101: mol_type = other DNA
ctcagcagcagccacaactccctcgcgcaacgacgaaacatcctttcatgcatagatccacggctacataattccttttggagtcactgtcgtcgaatggc

SEQ ID NO: 447 - DNA, 101 bp, synthetic construct
  source 1..101: mol_type = other DNA
ctcagcagcagccacaactccctcgcgcaacgcggaaagcccctttcatgcatagatccacggctacataattccttttggagtcactgtcgtcgaatggc

SEQ ID NO: 448 - DNA, 100 bp, synthetic construct
  source 1..100: mol_type = other DNA
ctcagcagcagccacaactccctcgcgcaactgccaaagaactttcatgcatagatccacggctacataattccttttggagtcactgtcgtcgaatggc

SEQ ID NO: 449 - DNA, 103 bp, synthetic construct
  source 1..103: mol_type = other DNA
ctcagcagcagccacaactccctcgcgcaacttcggaaagcagcctttcatgcatagatccacggctacataattccttttggagtcactgtcgtcgaatggc

SEQ ID NO: 450 - DNA, 99 bp, synthetic construct
  source 1..99: mol_type = other DNA
ctcagcagcagccacaactccctcgcgcaacattgaaagtcgttcatgcatagatccacggctacataattccttttggagtcactgtcgtcgaatggc

SEQ ID NO: 451 - DNA, 100 bp, synthetic construct
  source 1..100: mol_type = other DNA
ctcagcagcagccacaactccctcgcgcaacttcgaaagtgacttcatgcatagatccacggctacataattccttttggagtcactgtcgtcgaatggc

SEQ ID NO: 452 - DNA, 98 bp, synthetic construct
  source 1..98: mol_type = other DNA
ctcagcagcagccacaactccctcgcgcaacgatgattcctttcatgcatagatccacggctacataattccttttggagtcactgtcgtcgaatggc

SEQ ID NO: 453 - DNA, 99 bp, synthetic construct
  source 1..99: mol_type = other DNA
ctcagcagcagccacaactccctcgcgcaactgcaaaagtccatcatgcatagatccacggctacataattccttttggagtcactgtcgtcgaatggc

SEQ ID NO: 454 - DNA, 101 bp, synthetic construct
  source 1..101: mol_type = other DNA
ctcagcagcagccacaactccctcgcgcaactttgaaccgtccttccatgcatagatccacggctacataattccttttggagtcactgtcgtcgaatggc

SEQ ID NO: 455 - DNA, 99 bp, synthetic construct
  source 1..99: mol_type = other DNA
ctcagcagcagccacaactccctcgcgcaactttgatgtgatttcatgcatagatccacggctacataattccttttggagtcactgtcgtcgaatggc

SEQ ID NO: 456 - DNA, 99 bp, synthetic construct
  source 1..99: mol_type = other DNA
ctcagcagcagccacaactccctcgcgcaactcctgaaagtgttcatgcatagatccacggctacataattccttttggagtcactgtcgtcgaatggc

SEQ ID NO: 457 - DNA, 100 bp, synthetic construct
  source 1..100: mol_type = other DNA
ctcagcagcagccacaactccctcgcgcaactttacaaaagtgttcatgcatagatccacggctacataattccttttggagtcactgtcgtcgaatggc

SEQ ID NO: 458 - DNA, 99 bp, synthetic construct
  source 1..99: mol_type = other DNA
ctcagcagcagccacaactccctcgcgcaactttgacgtagtttcatgcatagatccacggctacataattccttttggagtcactgtcgtcgaatggc

SEQ ID NO: 459 - DNA, 100 bp, synthetic construct
  source 1..100: mol_type = other DNA
ctcagcagcagccacaactccctcgcgcaacggtgaaagtcaattcatgcatagatccacggctacataattccttttggagtcactgtcgtcgaatggc

SEQ ID NO: 460 - DNA, 100 bp, synthetic construct
  source 1..100: mol_type = other DNA
ctcagcagcagccacaactccctcgcgcaactgcgacgctcctttcatgcatagatccacggctacataattccttttggagtcactgtcgtcgaatggc

SEQ ID NO: 461 - DNA, 99 bp, synthetic construct
  source 1..99: mol_type = other DNA
ctcagcagcagccacaactccctcgcgcaactttgctaagtcatcatgcatagatccacggctacataattccttttggagtcactgtcgtcgaatggc

SEQ ID NO: 462 - DNA, 100 bp, synthetic construct
  source 1..100: mol_type = other DNA
ctcagcagcagccacaactccctcgcgcaactgtgaaagtgatttcatgcatagatccacggctacataattccttttggagtcactgtcgtcgaatggc

SEQ ID NO: 463 - DNA, 100 bp, synthetic construct
  source 1..100: mol_type = other DNA
ctcagcagcagccacaactccctcgcgcaactttgaaagtggtttcatgcatagatccacggctacataattccttttggagtcactgtcgtcgaatggc

SEQ ID NO: 464 - DNA, 101 bp, synthetic construct
  source 1..101: mol_type = other DNA
ctcagcagcagccacaactccctcgcgcaactttgcacaagtcgttcatgcatagatccacggctacataattccttttggagtcactgtcgtcgaatggc

SEQ ID NO: 465 - DNA, 100 bp, synthetic construct
  source 1..100: mol_type = other DNA
ctcagcagcagccacaactccctcgcgcaactgagaaacccctttcatgcatagatccacggctacataattccttttggagtcactgtcgtcgaatggc

SEQ ID NO: 466 - DNA, 103 bp, synthetic construct
  source 1..103: mol_type = other DNA
ctcagcagcagccacaactccctcgcgcaactttgatctagtgactttcatgcatagatccacggctacataattccttttggagtcactgtcgtcgaatggc

SEQ ID NO: 467 - DNA, 100 bp, synthetic construct
  source 1..100: mol_type = other DNA
ctcagcagcagccacaactccctcgcgcaacggggaaagtccttccatgcatagatccacggctacataattccttttggagtcactgtcgtcgaatggc

SEQ ID NO: 468 - DNA, 100 bp, synthetic construct
  source 1..100: mol_type = other DNA
ctcagcagcagccacaactccctcgcgcaactttgatcgtcggttcatgcatagatccacggctacataattccttttggagtcactgtcgtcgaatggc

SEQ ID NO: 469 - DNA, 101 bp, synthetic construct
  source 1..101: mol_type = other DNA
ctcagcagcagccacaactccctcgcgcaactcatgatcctcctttcatgcatagatccacggctacataattccttttggagtcactgtcgtcgaatggc

SEQ ID NO: 470 - DNA, 100 bp, synthetic construct
  source 1..100: mol_type = other DNA
ctcagcagcagccacaactccctcgcgcaactttgaccctcctttcatgcatagaaccacggctacataattccttttggagtcactgtcgtcgaatggc

SEQ ID NO: 471 - DNA, 100 bp, synthetic construct
  source 1..100: mol_type = other DNA
ctcagcagcagccacaactccctcgcgcaactttgaaagtgacttcatgcatagatccacggctacataattccttttggagtcactgtcgtcgaatggc

SEQ ID NO: 472 - DNA, 100 bp, synthetic construct
  source 1..100: mol_type = other DNA
ctcagcagcagccacaactccctcgcgcaactttgtccgggctttcatgcatagatccacggctacataattccttttggagtcactgtcgtcgaatggc

SEQ ID NO: 473 - DNA, 100 bp, synthetic construct
  source 1..100: mol_type = other DNA
ctcagcagcagccacaactccctcgcgcaacttcataagtccgggcatgcatagatccacggctacataattccttttggagtcactgtcgtcgaatggc

SEQ ID NO: 474 - DNA, 100 bp, synthetic construct
  source 1..100: mol_type = other DNA
ctcagcagcagccacaactccctcgcgcaactcatgatgtcctttcatgcatagatccacggctacataattccttttggagtcactgtcgtcgaatggc

SEQ ID NO: 475 - DNA, 99 bp, synthetic construct
  source 1..99: mol_type = other DNA
ctcagcagcagccacaactccctcgcgcaactttgttagtccgtcatgcatagatccacggctacataattccttttggagtcactgtcgtcgaatggc

SEQ ID NO: 476 - DNA, 100 bp, synthetic construct
  source 1..100: mol_type = other DNA
ctcagcagcagccacaactccctcgcgcaacttatcaagtcggatcatgcatagatccacggctacataattccttttggagtcactgtcgtcgaatggc

SEQ ID NO: 477 - DNA, 98 bp, synthetic construct
  source 1..98: mol_type = other DNA
ctcagcagcagccacaactccctcgcgcaacttcaagtcagttcatgcatagatccacggctacataattccttttggagtcactgtcgtcgaatggc

SEQ ID NO: 478 - DNA, 98 bp, synthetic construct
  source 1..98: mol_type = other DNA
ctcagcagcagccacaactccctcgcgcaactttcagtcctttgatgcatagatccacggctacataattccttttggagtcactgtcgtcgaatggc

SEQ ID NO: 479 - DNA, 99 bp, synthetic construct
  source 1..99: mol_type = other DNA
ctcagcagcagccacaactccctcgcgcaactttgtaagtcattcatgcatagatccacggctacataattccttttggagtcactgtcgtcgaatggc

SEQ ID NO: 480 - DNA, 96 bp, synthetic construct
  source 1..96: mol_type = other DNA
ctcagcagcagccacaactccctcgcgcaactttgcgtcgtcatgcatagatccacggctacataattccttttggagtcactgtcgtcgaatggc

SEQ ID NO: 481 - DNA, 102 bp, synthetic construct
  source 1..102: mol_type = other DNA
ctcagcagcagccacaactccctcgcgcaacttgtggaaagtcgattcatgcatagatccacggctacataattccttttggagtcactgtcgtcgaatggc

SEQ ID NO: 482 - DNA, 98 bp, synthetic construct
  source 1..98: mol_type = other DNA
ctcagcagcagccacaactccctcgcgcaactttgatgtcgttcatgcatagatccacggctacataattccttttggagtcactgtcgtcgaatggc

SEQ ID NO: 483 - DNA, 102 bp, synthetic construct
  source 1..102: mol_type = other DNA
ctcagcagcagccacaactccctcgcgcaacgatgaaatcttcctttcatgcatagatccacggctacataattccttttggagtcactgtcgtcgaatggc

SEQ ID NO: 484 - DNA, 100 bp, synthetic construct
  source 1..100: mol_type = other DNA
ctcagcagcagccacaactccctcgcgcaacggtgaaagtcctttcatgcatagatctacggctacataattccttttggagtcactgtcgtcgaatggc

SEQ ID NO: 485 - DNA, 101 bp, synthetic construct
  source 1..101: mol_type = other DNA
ctcagcagcagccacaactccctcgcgcaacttatagaaagtcctgcatgcatagatccacggctacataattccttttggagtcactgtcgtcgaatggc

SEQ ID NO: 486 - DNA, 99 bp, synthetic construct
  source 1..99: mol_type = other DNA
ctcagcagcagccacaactccctcgcgcaactttgaaaggctttcatgcatagatccacggctacataattccttttggagtcactgtcgtcgaatggc

SEQ ID NO: 487 - DNA, 100 bp, synthetic construct
  source 1..100: mol_type = other DNA
ctcagcagcagccacaactccctcgcgcaactccaaaacgcctttcatgcatagatccacggctacataattccttttggagtcactgtcgtcgaatggc

SEQ ID NO: 488 - DNA, 98 bp, synthetic construct
  source 1..98: mol_type = other DNA
ctcagcagcagccacaactccctcgcgcaactttcaagtcattcatgcatagatccacggctacataattccttttggagtcactgtcgtcgaatggc

SEQ ID NO: 489 - DNA, 99 bp, synthetic construct
  source 1..99: mol_type = other DNA
ctcagcagcagccacaactccctcgcgcaacttcaaagtaatttcatgcatagatccacggctacataattccttttggagtcactgtcgtcgaatggc

SEQ ID NO: 490 - DNA, 102 bp, synthetic construct
  source 1..102: mol_type = other DNA
ctcagcagcagccacaactccctcgcgcaactttacaaaagcactttcatgcatagatccacggctacataattccttttggagtcactgtcgtcgaatggc

SEQ ID NO: 491 - DNA, 100 bp, synthetic construct
  source 1..100: mol_type = other DNA
ctcagcagcagccacaactccctcgcgcaacgaagaacttcctttcatgcatagatccacggctacataattccttttggagtcactgtcgtcgaatggc

SEQ ID NO: 492 - DNA, 101 bp, synthetic construct
  source 1..101: mol_type = other DNA
ctcagcagcagccacaactccctcgcgcaacttacaaacctcctttcatgcatagatccacggctacataattccttttggagtcactgtcgtcgaatggc

SEQ ID NO: 493 - DNA, 99 bp, synthetic construct
  source 1..99: mol_type = other DNA
ctcagcagcagccacaactccctcgcgcaacattgacgtcctttcatgcatagatccacggctacataattccttttggagtcactgtcgtcgaatggc

SEQ ID NO: 494 - DNA, 102 bp, synthetic construct
  source 1..102: mol_type = other DNA
ctcagcagcagccacaactccctcgcgcaacgctgaaagtccgggttcatgcatagatccacggctacataattccttttggagtcactgtcgtcgaatggc

SEQ ID NO: 495 - DNA, 102 bp, synthetic construct
  source 1..102: mol_type = other DNA
ctcagcagcagccacaactccctcgcgcaactttgaaagcagcagttcatgcatagatccacggctacataattccttttggagtcactgtcgtcgaatggc

SEQ ID NO: 496 - DNA, 101 bp, synthetic construct
  source 1..101: mol_type = other DNA
ctcagcagcagccacaactccctcgcgcaacttgcgaaagtaacttcatgcatagatccacggctacataattccttttggagtcactgtcgtcgaatggc

SEQ ID NO: 497 - DNA, 98 bp, synthetic construct
  source 1..98: mol_type = other DNA
ctcagcagcagccacaactccctcgcgcaactttgaaagtgttcatgcatagatccacggctacataattccttttggagtcactgtcgtcgaatggc

SEQ ID NO: 498 - DNA, 98 bp, synthetic construct
  source 1..98: mol_type = other DNA
ctcagcagcagccacaactccctcgcgcaactttgtgtcctttcatgcatagatccacggctacataattccttttggagtcactgtcgtcgaatggc

SEQ ID NO: 499 - DNA, 99 bp, synthetic construct
  source 1..99: mol_type = other DNA
ctcagcagcagccacaactccctcgcgcaacttcaagtggctttcatgcatagatccacggctacataattccttttggagtcactgtcgtcgaatggc

SEQ ID NO: 500 - DNA, 102 bp, synthetic construct
  source 1..102: mol_type = other DNA
ctcagcagcagccacaactccctcgcgcaactttgattagtccgattcatgcatagatccacggctacataattccttttggagtcactgtcgtcgaatggc

SEQ ID NO: 501 - DNA, 100 bp, synthetic construct
  source 1..100: mol_type = other DNA
ctcagcagcagccacaactccctcgcgcaacttcccaagtcctttcatgcatagatccacggctacataattccttttggagtcactgtcgtcgaatggc

SEQ ID NO: 502 - DNA, 102 bp, synthetic construct
  source 1..102: mol_type = other DNA
ctcagcagcagccacaactccctcgcgcaacattgtccaagtcctttcatgcatagatccacggctacataattccttttggagtcactgtcgtcgaatggc

SEQ ID NO: 503 - DNA, 98 bp, synthetic construct
  source 1..98: mol_type = other DNA
ctcagcagcagccacaactccctcgcgcaacttcgaaagtgttcatgcatagatccacggctacataattccttttggagtcactgtcgtcgaatggc

SEQ ID NO: 504 - DNA, 99 bp, synthetic construct
  source 1..99: mol_type = other DNA
ctcagcagcagccacaactccctcgcgcaactttgtttgtcattcatgcatagatccacggctacataattccttttggagtcactgtcgtcgaatggc

SEQ ID NO: 505 - DNA, 102 bp, synthetic construct
  source 1..102: mol_type = other DNA
ctcagcagcagccacaactccctcgcgcaactttgaaactatcctttcatgcatagatccacggctacataattccttttggagtcactgtcgtcgaatggc

SEQ ID NO: 506 - DNA, 102 bp, synthetic construct
  source 1..102: mol_type = other DNA
ctcagcagcagccacaactccctcgcgcaactttgtccaagtgctttcatgcatagatccacggctacataattccttttggagtcactgtcgtcgaatggc

SEQ ID NO: 507 - DNA, 102 bp, synthetic construct
  source 1..102: mol_type = other DNA
ctcagcagcagccacaactccctcgcgcaactttgaactcgtcctttcatgcatagatccacggctacataattccttttggagtcactgtcgtcgaatggc

SEQ ID NO: 508 - DNA, 104 bp, synthetic construct
  source 1..104: mol_type = other DNA
ctcagcagcagccacaactccctcgcgcaacttaaagaaatactcctttcatgcatagatccacggctacataattccttttggagtcactgtcgtcgaatggc

SEQ ID NO: 509 - DNA, 98 bp, synthetic construct
  source 1..98: mol_type = other DNA
ctcagcagcagccacaactccctcgcgcaacttgtaaagtccacatgcatagatccacggctacataattccttttggagtcactgtcgtcgaatggc

SEQ ID NO: 510 - DNA, 100 bp, synthetic construct
  source 1..100: mol_type = other DNA
ctcagcagcagccacaactccctcgcgcaactttgaaaggagtttcatgcatagatccacggctacataattccttttggagtcactgtcgtcgaatggc

SEQ ID NO: 511 - DNA, 100 bp, synthetic construct
  source 1..100: mol_type = other DNA
ctcagcagcagccacaactccctcgcgcaacattgaaagtcatttcatgcatagatccacggctacataattccttttggagtcactgtcgtcgaatggc

SEQ ID NO: 512 - DNA, 98 bp, synthetic construct
  source 1..98: mol_type = other DNA
ctcagcagcagccacaactccctcgcgcaactttgactgctttcatgcatagatccacggctacataattccttttggagtcactgtcgtcgaatggc

SEQ ID NO: 513 - DNA, 98 bp, synthetic construct
  source 1..98: mol_type = other DNA
ctcagcagcagccacaactccctcgcgcaactttgtgccctttcatgcatagatccacggctacataattccttttggagtcactgtcgtcgaatggc

SEQ ID NO: 514 - DNA, 100 bp, synthetic construct
  source 1..100: mol_type = other DNA
ctcagcagcagccacaactccctcgcgcaacttaaaaagtgaattcatgcatagatccacggctacataattccttttggagtcactgtcgtcgaatggc

SEQ ID NO: 515 - DNA, 99 bp, synthetic construct
  source 1..99: mol_type = other DNA
ctcagcagcagccacaactccctcgcgcaactttgaaagtcgttcatgcatagatccacggctacataattccttttggagtcactgtcgtcgaatggc

SEQ ID NO: 516 - DNA, 102 bp, synthetic construct
  source 1..102: mol_type = other DNA
ctcagcagcagccacaactccctcgcgcaacgacgaaagatacctttcatgcatagatccacggctacataattccttttggagtcactgtcgtcgaatggc

SEQ ID NO: 517 - DNA, 100 bp, synthetic construct
  source 1..100: mol_type = other DNA
ctcagcagcagccacaactccctcgcgcaacttaaaagcgcctttcatgcatagatccacggctacataattccttttggagtcactgtcgtcgaatggc

SEQ ID NO: 518 - DNA, 101 bp, synthetic construct
  source 1..101: mol_type = other DNA
ctcagcagcagccacaactccctcgcgcaacttgtaaagtccgattcatgcatagatccacggctacataattccttttggagtcactgtcgtcgaatggc

SEQ ID NO: 519 - DNA, 100 bp, synthetic construct
  source 1..100: mol_type = other DNA
ctcagcagcagccacaactccctcgcgcaacattgaatttcctttcatgcatagatccacggctacataattccttttggagtcactgtcgtcgaatggc

SEQ ID NO: 520 - DNA, 98 bp, synthetic construct
  source 1..98: mol_type = other DNA
ctcagcagcagccacaactccctcgcgcaacttttcaagtcatcatgcatagatccacggctacataattccttttggagtcactgtcgtcgaatggc

SEQ ID NO: 521 - DNA, 100 bp, synthetic construct
  source 1..100: mol_type = other DNA
ctcagcagcagccacaactccctcgcgcaactcggaaggtcctttcatgcatagatccacggctacataattccttttggagtcactgtcgtcgaatggc

SEQ ID NO: 522 - DNA, 102 bp, synthetic construct
  source 1..102: mol_type = other DNA
ctcagcagcagccacaactccctcgcgcaactttgactgtccgcgttcatgcatagatccacggctacataattccttttggagtcactgtcgtcgaatggc

SEQ ID NO: 523 - DNA, 99 bp, synthetic construct
  source 1..99: mol_type = other DNA
ctcagcagcagccacaactccctcgcgcaactttgaaagtatttcatgcatagatccacggctacataattccttttggagtcactgtcgtcgaatggc

SEQ ID NO: 524 - DNA, 101 bp, synthetic construct
  source 1..101: mol_type = other DNA
ctcagcagcagccacaactccctcgcgcaactttgaaagaacttagcatgcatagatccacggctacataattccttttggagtcactgtcgtcgaatggc

SEQ ID NO: 525 - DNA, 102 bp, synthetic construct
  source 1..102: mol_type = other DNA
ctcagcagcagccacaactccctcgcgcaacttacagaaataactttcatgcatagatccacggctacataattccttttggagtcactgtcgtcgaatggc

SEQ ID NO: 526 - DNA, 98 bp, synthetic construct
  source 1..98: mol_type = other DNA
ctcagcagcagccacaactccctcgcgcaactcggattcctttcatgcatagatccacggctacataattccttttggagtcactgtcgtcgaatggc

SEQ ID NO: 527 - DNA, 100 bp, synthetic construct
  source 1..100: mol_type = other DNA
ctcagcagcagccacaactccctcgcgcaactttctcagtgctttcatgcatagatccacggctacataattccttttggagtcactgtcgtcgaatggc

SEQ ID NO: 528 - DNA, 97 bp, synthetic construct
  source 1..97: mol_type = other DNA
ctcagcagcagccacaactccctcgcgcaacttttagtcgttcatgcatagatccacggctacataattccttttggagtcactgtcgtcgaatggc

SEQ ID NO: 529 - DNA, 100 bp, synthetic construct
  source 1..100: mol_type = other DNA
ctcagcagcagccacaactccctcgcgcaactggaaaaggagtttcatgaaccgatccacggctatcatactcctttacaccacactgtcgtcgaatggc

SEQ ID NO: 530 - DNA, 98 bp, synthetic construct
  source 1..98: mol_type = other DNA
ctcagcagcagccacaactccctcgcgcaactttgtagtcgttcatgcatagatccacggctacataattccttttggagtcactgtcgtcgaatggc

SEQ ID NO: 531 - DNA, 100 bp, synthetic construct
  source 1..100: mol_type = other DNA
ctcagcagcagccacaactccctcgcgcaacttagcttgtcctttcatgcatagatccacggctacataattccttttggagtcactgtcgtcgaatggc

SEQ ID NO: 532 - DNA, 101 bp, synthetic construct
  source 1..101: mol_type = other DNA
ctcagcagcagccacaactccctcgcgcaactttgaccagtccttgcatgcatagatccacggctacataattccttttggagtcactgtcgtcgaatggc

SEQ ID NO: 533 - DNA, 101 bp, synthetic construct
  source 1..101: mol_type = other DNA
ctcagcagcagccacaactccctcgcgcaactcatgaaattactttcatgcatagatccacggctacataattccttttggagtcactgtcgtcgaatggc

SEQ ID NO: 534 - DNA, 100 bp, synthetic construct
  source 1..100: mol_type = other DNA
ctcagcagcagccacaactccctcgcgcaacttcgaaagggctttcatgcatagatccacggctacataattccttttggagtcactgtcgtcgaatggc

SEQ ID NO: 535 - DNA, 100 bp, synthetic construct
  source 1..100: mol_type = other DNA
ctcagcagcagccacaactccctcgcgcaactttgaaagtcagttcatgcatagatccacggctacataattccttttggagtcactgtcgtcgaatggc

SEQ ID NO: 536 - DNA, 100 bp, synthetic construct
  source 1..100: mol_type = other DNA
ctcagcagcagccacaactccctcgcgcaactttgaatttaatttcatgcatagatccacggctacataattccttttggagtcactgtcgtcgaatggc

SEQ ID NO: 537 - DNA, 102 bp, synthetic construct
  source 1..102: mol_type = other DNA
ctcagcagcagccacaactccctcgcgcaacttaaaaaacttcctttcatgcatagatccacggctacataattccttttggagtcactgtcgtcgaatggc

SEQ ID NO: 538 - DNA, 97 bp, synthetic construct
  source 1..97: mol_type = other DNA
ctcagcagcagccacaactccctcgcgcaactttcgtagattcatgtcgacatccacggctatcatactcctttacaccacactgtcgtcgaatggc

SEQ ID NO: 539 - DNA, 97 bp, synthetic construct
  source 1..97: mol_type = other DNA
ctcagcagcagccacaactccctcgcgcaactttgtgtatttcatgcatagatccacggctacataattccttttggagtcactgtcgtcgaatggc

SEQ ID NO: 540 - DNA, 99 bp, synthetic construct
  source 1..99: mol_type = other DNA
ctcagcagcagccacaactccctcgcgcaacttcaaagtcctttcatgcatagatccacggctacataattccttttggagtcactgtcgtcgaatggc

SEQ ID NO: 541 - DNA, 100 bp, synthetic construct
  source 1..100: mol_type = other DNA
ctcagcagcagccacaactccctcgcgcaactttgaaactaatttcatgcatagatccacggctacataattccttttggagtcactgtcgtcgaatggc

SEQ ID NO: 542 - DNA, 98 bp, synthetic construct
  source 1..98: mol_type = other DNA
ctcagcagcagccacaactccctcgcgcaactttcagatatttcatgcatagatccacggctacataattccttttggagtcactgtcgtcgaatggc

SEQ ID NO: 543 - DNA, 100 bp, synthetic construct
  source 1..100: mol_type = other DNA
ctcagcagcagccacaactccctcgcgcaactatgaaggtcctttcatgcatagatccacggctacataattccttttggagtcactgtcgtcgaatggc

SEQ ID NO: 544 - DNA, 101 bp, synthetic construct
  source 1..101: mol_type = other DNA
ctcagcagcagccacaactccctcgcgcaacgaagaactgtcctttcatgcatagatccacggctacataattccttttggagtcactgtcgtcgaatggc

SEQ ID NO: 545 - DNA, 100 bp, synthetic construct
  source 1..100: mol_type = other DNA
ctcagcagcagccacaactccctcgcgcaactttgaaagtgctttcatgcatagatccacggctacataattccttttggagtcactgtcgtcgaatggc

SEQ ID NO: 546 - DNA, 101 bp, synthetic construct
  source 1..101: mol_type = other DNA
ctcagcagcagccacaactccctcgcgcaacttgctaaggagtttcatgatagtcatccacggctatcatactcctttacaccacactgtcgtcgaatggc

SEQ ID NO: 547 - DNA, 100 bp, synthetic construct
  source 1..100: mol_type = other DNA
ctcagcagcagccacaactccctcgcgcaacggagaaagggatttcatgacaccatccacggctatcatactcctttacaccacactgtcgtcgaatggc

SEQ ID NO: 548 - DNA, 101 bp, synthetic construct
  source 1..101: mol_type = other DNA
ctcagcagcagccacaactccctcgcgcaactttgatcagtccgggcatgcatagatccacggctacataattccttttggagtcactgtcgtcgaatggc

SEQ ID NO: 549 - DNA, 101 bp, synthetic construct
  source 1..101: mol_type = other DNA
ctcagcagcagccacaactccctcgcgcaactttgcaagtcccattcatgcatagatccacggctacataattccttttggagtcactgtcgtcgaatggc

SEQ ID NO: 550 - DNA, 102 bp, synthetic construct
  source 1..102: mol_type = other DNA
ctcagcagcagccacaactccctcgcgcaactttctcaaagtcctttcatgcatagatccacggctacataattccttttggagtcactgtcgtcgaatggc

SEQ ID NO: 551 - DNA, 100 bp, synthetic construct
  source 1..100: mol_type = other DNA
ctcagcagcagccacaactccctcgcgcaactgtgaaaatcctttcatgcatagatccacggctacataattccttttggagtcactgtcgtcgaatggc

SEQ ID NO: 552 - DNA, 99 bp, synthetic construct
  source 1..99: mol_type = other DNA
ctcagcagcagccacaactccctcgcgcaactttttaagtcattcatgcatagatccacggctacataattccttttggagtcactgtcgtcgaatggc

SEQ ID NO: 553 - DNA, 98 bp, synthetic construct
  source 1..98: mol_type = other DNA
ctcagcagcagccacaactccctcgcgcaactttgaaagatttcatgcatagatccacggctacataattccttttggagtcactgtcgtcgaatggc

SEQ ID NO: 554 - DNA, 99 bp, synthetic construct
  source 1..99: mol_type = other DNA
ctcagcagcagccacaactccctcgcgcaactttgaaagtcctggcatgcatagaccacggctacataattccttttggagtcactgtcgtcgaatggc

SEQ ID NO: 555 - DNA, 100 bp, synthetic construct
  source 1..100: mol_type = other DNA
ctcagcagcagccacaactccctcgcgcaactttgtttggggtttcatgatgtcatccacggctatcatactcctttacaccacactgtcgtcgaatggc

SEQ ID NO: 556 - DNA, 99 bp, synthetic construct
  source 1..99: mol_type = other DNA
ctcagcagcagccacaactccctcgcgcaactttccaaagtcatcatgcatagatccacggctacataattccttttggagtcactgtcgtcgaatggc

SEQ ID NO: 557 - DNA, 98 bp, synthetic construct
  source 1..98: mol_type = other DNA
ctcagcagcagccacaactccctcgcgcaactttgattcatttcatgcatagatccacggctacataattccttttggagtcactgtcgtcgaatggc

SEQ ID NO: 558 - DNA, 103 bp, synthetic construct
  source 1..103: mol_type = other DNA
ctcagcagcagccacaactccctcgcgcaacttgtcgaaagtcctttcatgcatagatggcacggctacataattccttttggagtcactgtcgtcgaatggc

SEQ ID NO: 559 - DNA, 101 bp, synthetic construct
  source 1..101: mol_type = other DNA
ctcagcagcagccacaactccctcgcgcaactagctaaaggagtttcatgaatagccccacggctatcatactcctttacaccacactgtcgtcgaatggc

SEQ ID NO: 560 - DNA, 100 bp, synthetic construct
  source 1..100: mol_type = other DNA
ctcagcagcagccacaactccctcgcgcaacgctgaaagtcctttcatgcatagatccacggctacataattccttttggagtcactgtcgtcgaatggc

SEQ ID NO: 561 - DNA, 100 bp, synthetic construct
  source 1..100: mol_type = other DNA
ctcagcagcagccacaactccctcgcgcaactgccaaaggagtttcatgtacccatccacggctatcatactcctttacaccacactgtcgtcgaatggc

SEQ ID NO: 562 - DNA, 98 bp, synthetic construct
  source 1..98: mol_type = other DNA
ctcagcagcagccacaactccctcgcgcaacttcaagtcctttcatgcatagatccacggctacataattccttttggagtcactgtcgtcgaatggc

SEQ ID NO: 563 - DNA, 100 bp, synthetic construct
  source 1..100: mol_type = other DNA
ctcagcagcagccacaactccctcgcgcaactttgaaagacacatcatgcatagatccacggctacataattccttttggagtcactgtcgtcgaatggc

SEQ ID NO: 564 - DNA, 100 bp, synthetic construct
  source 1..100: mol_type = other DNA
ctcagcagcagccacaactccctcgcgcaactacataaggagtttcatcgttacatccacggctatcatactcctttacaccacactgtcgtcgaatggc

SEQ ID NO: 565 - DNA, 102 bp, synthetic construct
  source 1..102: mol_type = other DNA
ctcagcagcagccacaactccctcgcgcaactttgaaataatcacttcatgcatagatccacggctacataattccttttggagtcactgtcgtcgaatggc

SEQ ID NO: 566 - DNA, 101 bp, synthetic construct
  source 1..101: mol_type = other DNA
ctcagcagcagccacaactccctcgcgcaactttgaaagcaccaatcatgcatagatccacggctacataattccttttggagtcactgtcgtcgaatggc

SEQ ID NO: 567 - DNA, 102 bp, synthetic construct
  source 1..102: mol_type = other DNA
ctcagcagcagccacaactccctcgcgcaactttgaactgtaactttcatgcatagatccacggctacataattccttttggagtcactgtcgtcgaatggc

SEQ ID NO: 568 - DNA, 100 bp, synthetic construct
  source 1..100: mol_type = other DNA
ctcagcagcagccacaactccctcgcgcaacttcgaaacgcctttcatgcatagatccacggctacataattccttttggagtcactgtcgtcgaatggc

SEQ ID NO: 569 - DNA, 97 bp, synthetic construct
  source 1..97: mol_type = other DNA
ctcagcagcagccacaactccctcgcgcaactttgtagatttcatgcatagatccacggctacataattccttttggagtcactgtcgtcgaatggc

SEQ ID NO: 570 - DNA, 102 bp, synthetic construct
  source 1..102: mol_type = other DNA
ctcagcagcagccacaactccctcgcgcaactttgaatcgtccgattcatgcatagatccacggctacataattccttttggagtcactgtcgtcgaatggc

SEQ ID NO: 571 - DNA, 102 bp, synthetic construct
  source 1..102: mol_type = other DNA
ctcagcagcagccacaactccctcgcgcaactttgactatggggtttcatccttacatccacggctatcatactcctttacaccacactgtcgtcgaatggc

SEQ ID NO: 572 - DNA, 101 bp, synthetic construct
  source 1..101: mol_type = other DNA
ctcagcagcagccacaactccctcgcgcaactttgctaaaccctttcatgcatagatccacggctacataattccttttggagtcactgtcgtcgaatggc

SEQ ID NO: 573 - DNA, 100 bp, synthetic construct
  source 1..100: mol_type = other DNA
ctcagcagcagccacaactccctcgcgcaactgccaaaggagtttcatgcgcacatccacggctatcatactcctttacaccacactgtcgtcgaatggc

SEQ ID NO: 574 - DNA, 102 bp, synthetic construct
  source 1..102: mol_type = other DNA
ctcagcagcagccacaactccctcgcgcaacttaacaagcggtttcatgtcgctacatccacggctatcatactcctttacaccacactgtcgtcgaatggc

SEQ ID NO: 575 - DNA, 99 bp, synthetic construct
  source 1..99: mol_type = other DNA
ctcagcagcagccacaactccctcgcgcaactttgaaagtcattcatgcatagacacacggctacataattccttttggagtcactgtcgtcgaatggc

SEQ ID NO: 576 - DNA, 98 bp, synthetic construct
  source 1..98: mol_type = other DNA
ctcagcagcagccacaactccctcgcgcaactttgactcacatcatgcatagatccacggctacataattccttttggagtcactgtcgtcgaatggc

SEQ ID NO: 577 - DNA, 100 bp, synthetic construct
  source 1..100: mol_type = other DNA
ctcagcagcagccacaactccctcgcgcaactttgaaagtgatttcatgaatagatccacggctatcatactcctttacaccacactgtcgtcgaatggc

SEQ ID NO: 578 - DNA, 102 bp, synthetic construct
  source 1..102: mol_type = other DNA
ctcagcagcagccacaactccctcgcgcaacggggaaagtagtatttcatgatgccatccacggctatcatactcctttacaccacactgtcgtcgaatggc

SEQ ID NO: 579 - DNA, 101 bp, synthetic construct
  source 1..101: mol_type = other DNA
ctcagcagcagccacaactccctcgcgcaacttctctaagtgggttcatgacctcatccacggctatcatactcctttacaccacactgtcgtcgaatggc

SEQ ID NO: 580 - DNA, 98 bp, synthetic construct
  source 1..98: mol_type = other DNA
ctcagcagcagccacaactccctcgcgcaactgtgaaagtcgtcatgcttacatccacggctatcatactcctttacaccacactgtcgtcgaatggc

SEQ ID NO: 581 - DNA, 98 bp, synthetic construct
  source 1..98: mol_type = other DNA
ctcagcagcagccacaactccctcgcgcaactttgtagtcattcatgcatagatccacggctacataattccttttggagtcactgtcgtcgaatggc

SEQ ID NO: 582 - DNA, 101 bp, synthetic construct
  source 1..101: mol_type = other DNA
ctcagcagcagccacaactccctcgcgcaacgaagaacccgcctttcatgctgacatccacggctatcatactcctttacaccacactgtcgtcgaatggc

SEQ ID NO: 583 - DNA, 102 bp, synthetic construct
  source 1..102: mol_type = other DNA
ctcagcagcagccacaactccctcgcgcaacttctataaaggaatttcatgaataaccccacggctatcatactcctttacaccacactgtcgtcgaatggc

SEQ ID NO: 584 - DNA, 99 bp, synthetic construct
  source 1..99: mol_type = other DNA
ctcagcagcagccacaactccctcgcgcaactttgaaaggggtttcatgcatagaccacggctacataattccttttggagtcactgtcgtcgaatggc

SEQ ID NO: 585 - DNA, 100 bp, synthetic construct
  source 1..100: mol_type = other DNA
ctcagcagcagccacaactccctcgcgcaactttgattgtcctgtcatgcatagatccacggctacataattccttttggagtcactgtcgtcgaatggc

SEQ ID NO: 586 - DNA, 96 bp, synthetic construct
  source 1..96: mol_type = other DNA
ctcagcagcagccacaactccctcgcgcaactttgcgtcctgatgcatagatccacggctacataattccttttggagtcactgtcgtcgaatggc

SEQ ID NO: 587 - DNA, 100 bp, synthetic construct
  source 1..100: mol_type = other DNA
ctcagcagcagccacaactccctcgcgcaactcgaaaagtgacttcattgctacatccacggctatcatactcctttacaccacactgtcgtcgaatggc

SEQ ID NO: 588 - DNA, 99 bp, synthetic construct
  source 1..99: mol_type = other DNA
ctcagcagcagccacaactccctcgcgcaactttgaaagtaacttcatgcatagaccacggctacataattccttttggagtcactgtcgtcgaatggc

SEQ ID NO: 589 - DNA, 99 bp, synthetic construct
  source 1..99: mol_type = other DNA
ctcagcagcagccacaactccctcgcgcaactttgaaagtggtttcatgcatagaccacggctacataattccttttggagtcactgtcgtcgaatggc

SEQ ID NO: 590 - DNA, 100 bp, synthetic construct
  source 1..100: mol_type = other DNA
ctcagcagcagccacaactccctcgcgcaactgtgaaagtcctttcatgcatagatccacggctacataattccttttggagtcactgtcgtcgaatggc

SEQ ID NO: 591 - DNA, 101 bp, synthetic construct
  source 1..101: mol_type = other DNA
ctcagcagcagccacaactccctcgcgcaactttgctaagtcctgtcatgcatagatccacggctacataattccttttggagtcactgtcgtcgaatggc

SEQ ID NO: 592 - DNA, 101 bp, synthetic construct
  source 1..101: mol_type = other DNA
ctcagcagcagccacaactccctcgcgcaactttctcaaagtccgtcatgactacatccacggctatcatactcctttacaccacactgtcgtcgaatggc

SEQ ID NO: 593 - DNA, 102 bp, synthetic construct
  source 1..102: mol_type = other DNA
ctcagcagcagccacaactccctcgcgcaactttgaccctccgcgttcatgcatagatccacggctacataattccttttggagtcactgtcgtcgaatggc

SEQ ID NO: 594 - DNA, 99 bp, synthetic construct
  source 1..99: mol_type = other DNA
ctcagcagcagccacaactccctcgcgcaactttgaaaggactttcatgcatagaccacggctacataattccttttggagtcactgtcgtcgaatggc

SEQ ID NO: 595 - DNA, 102 bp, synthetic construct
  source 1..102: mol_type = other DNA
ctcagcagcagccacaactccctcgcgcaactttgtttaagtcctctcatgcatagatccacggctacataattccttttggagtcactgtcgtcgaatggc

SEQ ID NO: 596 - DNA, 99 bp, synthetic construct
  source 1..99: mol_type = other DNA
ctcagcagcagccacaactccctcgcgcaactttgacgctccctcatgcatagatccacggctacataattccttttggagtcactgtcgtcgaatggc

SEQ ID NO: 597 - DNA, 100 bp, synthetic construct
  source 1..100: mol_type = other DNA
ctcagcagcagccacaactccctcgcgcaactttgacgcagtcgtcatgcatagatccacggctacataattccttttggagtcactgtcgtcgaatggc

SEQ ID NO: 598 - DNA, 98 bp, synthetic construct
  source 1..98: mol_type = other DNA
ctcagcagcagccacaactccctcgcgcaactttgaaagtgttcgtgcatagatccacggctacataattccttttggagtcactgtcgtcgaatggc

SEQ ID NO: 599 - DNA, 101 bp, synthetic construct
  source 1..101: mol_type = other DNA
ctcagcagcagccacaactccctcgcgcaactttgtcaagtccgttcatgcatagatccacggctacataattccttttggagtcactgtcgtcgaatggc

SEQ ID NO: 600 - DNA, 97 bp, synthetic construct
  source 1..97: mol_type = other DNA
ctcagcagcagccacaactccctcgcgcaacttcaagtgtttcatgaatcgatccacggctatcatactcctttacaccacactgtcgtcgaatggc

SEQ ID NO: 601 - DNA, 99 bp, synthetic construct
  source 1..99: mol_type = other DNA
ctcagcagcagccacaactccctcgcgcaactttgaaagtgtttcatgcatacaaccacggctatcatactcctttacaccacactgtcgtcgaatggc

SEQ ID NO: 602 - DNA, 100 bp, synthetic construct
  source 1..100: mol_type = other DNA
ctcagcagcagccacaactccctcgcgcaactttgaccgtcctttcatgcatagatccacggctacataattccttttggagtcactgtcgtcgaatggc

SEQ ID NO: 603 - DNA, 102 bp, synthetic construct
  source 1..102: mol_type = other DNA
ctcagcagcagccacaactccctcgcgcaactttgaaattcccgattcatgcatagatccacggctacataattccttttggagtcactgtcgtcgaatggc

SEQ ID NO: 604 - DNA, 100 bp, synthetic construct
  source 1..100: mol_type = other DNA
ctcagcagcagccacaactccctcgcgcaactacaaaaggggattcatccttacatccacggctatcatactcctttacaccacactgtcgtcgaatggc

SEQ ID NO: 605 - DNA, 100 bp, synthetic construct
  source 1..100: mol_type = other DNA
ctcagcagcagccacaactccctcgcgcaactttgcttggatgttcattgctacatccacggctatcatactcctttacaccacactgtcgtcgaatggc

SEQ ID NO: 606 - DNA, 98 bp, synthetic construct
  source 1..98: mol_type = other DNA
ctcagcagcagccacaactccctcgcgcaactttgaaaggtttcatgaatagatctacggctatcatactcctttacaccacactgtcgtcgaatggc

SEQ ID NO: 607 - DNA, 101 bp, synthetic construct
  source 1..101: mol_type = other DNA
ctcagcagcagccacaactccctcgcgcaactttgaaacagcttcccatgcatagatccacggctacataattccttttggagtcactgtcgtcgaatggc

SEQ ID NO: 608 - DNA, 99 bp, synthetic construct
  source 1..99: mol_type = other DNA
ctcagcagcagccacaactccctcgcgcaactttgaaagtccttaattgcatagaccacggctacataattccttttggagtcactgtcgtcgaatggc

SEQ ID NO: 609 - DNA, 99 bp, synthetic construct
  source 1..99: mol_type = other DNA
ctcagcagcagccacaactccctcgcgcaactttgtgtgatttcatgaataccctccacggctatcatactcctttacaccacactgtcgtcgaatggc

SEQ ID NO: 610 - DNA, 103 bp, synthetic construct
  source 1..103: mol_type = other DNA
ctcagcagcagccacaactccctcgcgcaacgtcgaccctcctttcatgaattgaccatccacggctatcatactcctttacaccacactgtcgtcgaatggc

SEQ ID NO: 611 - DNA, 98 bp, synthetic construct
  source 1..98: mol_type = other DNA
ctcagcagcagccacaactccctcgcgcaactttgaaagtattcatgcatagatctacggctacataattccttttggagtcactgtcgtcgaatggc

SEQ ID NO: 612 - DNA, 99 bp, synthetic construct
  source 1..99: mol_type = other DNA
ctcagcagcagccacaactccctcgcgcaacggtgaaagacctttcatgaatacaccacggctatcatactcctttacaccacactgtcgtcgaatggc

SEQ ID NO: 613 - DNA, 98 bp, synthetic construct
  source 1..98: mol_type = other DNA
ctcagcagcagccacaactccctcgcgcaactttcagtgatttcatgaatagatccacggctatcatactcctttacaccacactgtcgtcgaatggc

SEQ ID NO: 614 - DNA, 98 bp, synthetic construct
  source 1..98: mol_type = other DNA
ctcagcagcagccacaactccctcgcgcaactttgaaccctggaatgcatagatccacggctacataattccttttggagtcactgtcgtcgaatggc

SEQ ID NO: 615 - DNA, 99 bp, synthetic construct
  source 1..99: mol_type = other DNA
ctcagcagcagccacaactccctcgcgcaactttgtagtcctttcatgcatagatccacggctacataattccttttggagtcactgtcgtcgaatggc

SEQ ID NO: 616 - DNA, 100 bp, synthetic construct
  source 1..100: mol_type = other DNA
ctcagcagcagccacaactccctcgcgcaactttgaaagtgatttcatgcatagacacacggctacataattccttttggagtcactgtcgtcgaatggc

SEQ ID NO: 617 - DNA, 98 bp, synthetic construct
  source 1..98: mol_type = other DNA
ctcagcagcagccacaactccctcgcgcaactttgactcctagcatgcatagatccacggctacataattccttttggagtcactgtcgtcgaatggc

SEQ ID NO: 618 - DNA, 102 bp, synthetic construct
  source 1..102: mol_type = other DNA
ctcagcagcagccacaactccctcgcgcaactgccaaagtggggtttcatgtccacatccacggctatcatactcctttacaccacactgtcgtcgaatggc

SEQ ID NO: 619 - DNA, 97 bp, synthetic construct
  source 1..97: mol_type = other DNA
ctcagcagcagccacaactccctcgcgcaactttgaaagtcgtcatgttacaaccacggctatcatactcctttacaccacactgtcgtcgaatggc

SEQ ID NO: 620 - DNA, 101 bp, synthetic construct
  source 1..101: mol_type = other DNA
ctcagcagcagccacaactccctcgcgcaactttgttaagtcctttcgtgcatagatccacggctacataattccttttggagtcactgtcgtcgaatggc

SEQ ID NO: 621 - DNA, 99 bp, synthetic construct
  source 1..99: mol_type = other DNA
ctcagcagcagccacaactccctcgcgcaactttgtagtcgtttcatgcatagatccacggctacataattccttttggagtcactgtcgtcgaatggc

SEQ ID NO: 622 - DNA, 100 bp, synthetic construct
  source 1..100: mol_type = other DNA
ctcagcagcagccacaactccctcgcgcaactttgaaagtattccatgaataagatccacggctatcatactcctttacaccacactgtcgtcgaatggc

SEQ ID NO: 623 - DNA, 99 bp, synthetic construct
  source 1..99: mol_type = other DNA
ctcagcagcagccacaactccctcgcgcaactttgaaaggagtttcatgcatagaccacggctacataattccttttggagtcactgtcgtcgaatggc

SEQ ID NO: 624 - DNA, 101 bp, synthetic construct
  source 1..101: mol_type = other DNA
ctcagcagcagccacaactccctcgcgcaacttgttaagcatatttcatcactacatccacggctatcatactcctttacaccacactgtcgtcgaatggc

SEQ ID NO: 625 - DNA, 100 bp, synthetic construct
  source 1..100: mol_type = other DNA
ctcagcagcagccacaactccctcgcgcaacttgtcaagagatttcatctttacatccacggctatcatactcctttacaccacactgtcgtcgaatggc

SEQ ID NO: 626 - DNA, 98 bp, synthetic construct
  source 1..98: mol_type = other DNA
ctcagcagcagccacaactccctcgcgcaactttgaaaggctttcatgaatgcaccacggctatcatactcctttacaccacactgtcgtcgaatggc

SEQ ID NO: 627 - DNA, 99 bp, synthetic construct
  source 1..99: mol_type = other DNA
ctcagcagcagccacaactccctcgcgcaactttgaactacctacatgcatagatccacggctacataattccttttggagtcactgtcgtcgaatggc

SEQ ID NO: 628 - DNA, 102 bp, synthetic construct
  source 1..102: mol_type = other DNA
ctcagcagcagccacaactccctcgcgcaactttgcataagcactttcatgcatagatccacggctacataattccttttggagtcactgtcgtcgaatggc

SEQ ID NO: 629 - DNA, 103 bp, synthetic construct
  source 1..103: mol_type = other DNA
ctcagcagcagccacaactccctcgcgcaactttgaatgctgtggattcatgctcacatccacggctatcatactcctttacaccacactgtcgtcgaatggc

SEQ ID NO: 630 - DNA, 99 bp, synthetic construct
  source 1..99: mol_type = other DNA
ctcagcagcagccacaactccctcgcgcaactttgaaagtccagtcatgcatagaccacggctacataattccttttggagtcactgtcgtcgaatggc

SEQ ID NO: 631 - DNA, 100 bp, synthetic construct
  source 1..100: mol_type = other DNA
ctcagcagcagccacaactccctcgcgcaactttgacagccctttcatgcatagatccacggctacataattccttttggagtcactgtcgtcgaatggc

SEQ ID NO: 632 - DNA, 100 bp, synthetic construct
  source 1..100: mol_type = other DNA
ctcagcagcagccacaactccctcgcgcaactgtccaaggaatttcacttatacatccacggctatcatactcctttacaccacactgtcgtcgaatggc

SEQ ID NO: 633 - DNA, 99 bp, synthetic construct
  source 1..99: mol_type = other DNA
ctcagcagcagccacaactccctcgcgcaactttgaaagtcattcatgaatccatccacggctatcatactcctttacaccacactgtcgtcgaatggc

SEQ ID NO: 634 - DNA, 100 bp, synthetic construct
  source 1..100: mol_type = other DNA
ctcagcagcagccacaactccctcgcgcaacttcctaagcgggttcatagttacatccacggctatcatactcctttacaccacactgtcgtcgaatggc

SEQ ID NO: 635 - DNA, 98 bp, synthetic construct
  source 1..98: mol_type = other DNA
ctcagcagcagccacaactccctcgcgcaactttgaaagtcgttcatgaatgcaccacggctatcatactcctttacaccacactgtcgtcgaatggc

SEQ ID NO: 636 - DNA, 100 bp, synthetic construct
  source 1..100: mol_type = other DNA
ctcagcagcagccacaactccctcgcgcaactttgaaagtcattcatgaataagatccacggctatcatactcctttacaccacactgtcgtcgaatggc

SEQ ID NO: 637 - DNA, 101 bp, synthetic construct
  source 1..101: mol_type = other DNA
ctcagcagcagccacaactccctcgcgcaactttgaaagacccctttcatgcatagaccacggctacataattccttttggagtcactgtcgtcgaatggc

SEQ ID NO: 638 - DNA, 100 bp, synthetic construct
  source 1..100: mol_type = other DNA
ctcagcagcagccacaactccctcgcgcaacgacgaaaggtatttcatgattccatccacggctatcatactcctttacaccacactgtcgtcgaatggc

SEQ ID NO: 639 - DNA, 100 bp, synthetic construct
  source 1..100: mol_type = other DNA
ctcagcagcagccacaactccctcgcgcaactttgactgtatttcatgccatacatccacggctatcatactcctttacaccacactgtcgtcgaatggc

SEQ ID NO: 640 - DNA, 100 bp, synthetic construct
  source 1..100: mol_type = other DNA
ctcagcagcagccacaactccctcgcgcaactgctaaagcgatttcattgctacatccacggctatcatactcctttacaccacactgtcgtcgaatggc

SEQ ID NO: 641 - DNA, 99 bp, synthetic construct
  source 1..99: mol_type = other DNA
ctcagcagcagccacaactccctcgcgcaactttgaaatgactttcatgcatagaccacggctacataattccttttggagtcactgtcgtcgaatggc

SEQ ID NO: 642 - DNA, 99 bp, synthetic construct
  source 1..99: mol_type = other DNA
ctcagcagcagccacaactccctcgcgcaactttgaaagtatttcatgacgacaaccacggctatcatactcctttacaccacactgtcgtcgaatggc

SEQ ID NO: 643 - DNA, 100 bp, synthetic construct
  source 1..100: mol_type = other DNA
ctcagcagcagccacaactccctcgcgcaactttgtctgtcctttcatgcatagatccacggctacataattccttttggagtcactgtcgtcgaatggc

SEQ ID NO: 644 - DNA, 99 bp, synthetic construct
  source 1..99: mol_type = other DNA
ctcagcagcagccacaactccctcgcgcaactttgaaactactttcatgcatagaccacggctacataattccttttggagtcactgtcgtcgaatggc

SEQ ID NO: 645 - DNA, 100 bp, synthetic construct
  source 1..100: mol_type = other DNA
ctcagcagcagccacaactccctcgcgcaactttgccagtcctttcatgcatagacccacggctacataattccttttggagtcactgtcgtcgaatggc

SEQ ID NO: 646 - DNA, 97 bp, synthetic construct
  source 1..97: mol_type = other DNA
ctcagcagcagccacaactccctcgcgcaactttgtttcctttcatgtgcccatccacggcaatcgccgactttacaccacactgtcgtcgaatggc

SEQ ID NO: 647 - DNA, 98 bp, synthetic construct
  source 1..98: mol_type = other DNA
ctcagcagcagccacaactccctcgcgcaactttgaaaactttcatgcatagatcgacggctacataattccttttggagtcactgtcgtcgaatggc

SEQ ID NO: 648 - DNA, 99 bp, synthetic construct
  source 1..99: mol_type = other DNA
ctcagcagcagccacaactccctcgcgcaactttgaaagtcattcatgactacatccacggctatcatactcctttacaccacactgtcgtcgaatggc

SEQ ID NO: 649 - DNA, 100 bp, synthetic construct
  source 1..100: mol_type = other DNA
ctcagcagcagccacaactccctcgcgcaacttcaagtaagctttcatgcatacatccacggctatcatactcctttacaccacactgtcgtcgaatggc

SEQ ID NO: 650 - DNA, 99 bp, synthetic construct
  source 1..99: mol_type = other DNA
ctcagcagcagccacaactccctcgcgcaactttgaccacctttcatgctacacatccacggcaatcgccgactttacaccacactgtcgtcgaatggc

SEQ ID NO: 651 - DNA, 99 bp, synthetic construct
  source 1..99: mol_type = other DNA
ctcagcagcagccacaactccctcgcgcaactttgaaagtaatttcatgcatagaccacggctacataattccttttggagtcactgtcgtcgaatggc

SEQ ID NO: 652 - DNA, 100 bp, synthetic construct
  source 1..100: mol_type = other DNA
ctcagcagcagccacaactccctcgcgcaactcatgatgtcctttcatgaatacatctacggctatcatactcctttacaccacactgtcgtcgaatggc

SEQ ID NO: 653 - DNA, 98 bp, synthetic construct
  source 1..98: mol_type = other DNA
ctcagcagcagccacaactccctcgcgcaactttgaaagtcgttcatgaaccatccacggctatcatactcctttacaccacactgtcgtcgaatggc

SEQ ID NO: 654 - DNA, 100 bp, synthetic construct
  source 1..100: mol_type = other DNA
ctcagcagcagccacaactccctcgcgcaactctaaaagtgggttcatgcggccatccacggctatcatactcctttacaccacactgtcgtcgaatggc

SEQ ID NO: 655 - DNA, 97 bp, synthetic construct
  source 1..97: mol_type = other DNA
ctcagcagcagccacaactccctcgcgcaactttgaacgctttcctccatacatccacggcaatcgccgactttacaccacactgtcgtcgaatggc

SEQ ID NO: 656 - DNA, 97 bp, synthetic construct
  source 1..97: mol_type = other DNA
ctcagcagcagccacaactccctcgcgcaacttcccaagattcatccttacatccacggctatcatactcctttacaccacactgtcgtcgaatggc

SEQ ID NO: 657 - DNA, 101 bp, synthetic construct
  source 1..101: mol_type = other DNA
ctcagcagcagccacaactccctcgcgcaactttgtcaagtactttcatgcatagatccacggctacataattccttttggagtcactgtcgtcgaatggc

SEQ ID NO: 658 - DNA, 99 bp, synthetic construct
  source 1..99: mol_type = other DNA
ctcagcagcagccacaactccctcgcgcaactttgaaagtccaggcatgcatagaccacggctacataattccttttggagtcactgtcgtcgaatggc

SEQ ID NO: 659 - DNA, 99 bp, synthetic construct
  source 1..99: mol_type = other DNA
ctcagcagcagccacaactccctcgcgcaactttgaaagtgatttcatgcatagaccacggctacataattccttttggagtcactgtcgtcgaatggc

SEQ ID NO: 660 - DNA, 99 bp, synthetic construct
  source 1..99: mol_type = other DNA
ctcagcagcagccacaactccctcgcgcaactttgaaagatttcatgacttacttccacggctatcatactcctttacaccacactgtcgtcgaatggc

SEQ ID NO: 661 - DNA, 98 bp, synthetic construct
  source 1..98: mol_type = other DNA
ctcagcagcagccacaactccctcgcgcaactttgaaagtgtttcatgaaaccaccacggctatcatactcctttacaccacactgtcgtcgaatggc

SEQ ID NO: 662 - DNA, 100 bp, synthetic construct
  source 1..100: mol_type = other DNA
ctcagcagcagccacaactccctcgcgcaactttcttagtaggttcatgaatctctccacggctatcatactcctttacaccacactgtcgtcgaatggc

SEQ ID NO: 663 - DNA, 103 bp, synthetic construct
  source 1..103: mol_type = other DNA
ctcagcagcagccacaactccctcgcgcaactttgaattgcgtgggttcattgttacatccacggctatcatactcctttacaccacactgtcgtcgaatggc

SEQ ID NO: 664 - DNA, 99 bp, synthetic construct
  source 1..99: mol_type = other DNA
ctcagcagcagccacaactccctcgcgcaactttgactgtcgttcatgaatccatccacggctatcatactcctttacaccacactgtcgtcgaatggc

SEQ ID NO: 665 - DNA, 100 bp, synthetic construct
  source 1..100: mol_type = other DNA
ctcagcagcagccacaactccctcgcgcaactgttaaagtcctttcactcatacatctacggctatcatactcctttacaccacactgtcgtcgaatggc

SEQ ID NO: 666 - DNA, 101 bp, synthetic construct
  source 1..101: mol_type = other DNA
ctcagcagcagccacaactccctcgcgcaacgtcgattactcctttcatgaataaagccacggctatcatactcctttacaccacactgtcgtcgaatggc

SEQ ID NO: 667 - DNA, 100 bp, synthetic construct
  source 1..100: mol_type = other DNA
ctcagcagcagccacaactccctcgcgcaactttgaaagggctttcatgaatcgatccacggctatcatactcctttacaccacactgtcgtcgaatggc

SEQ ID NO: 668 - DNA, 98 bp, synthetic construct
  source 1..98: mol_type = other DNA
ctcagcagcagccacaactccctcgcgcaactttgaaagtgtttcatgaaccatccacggctatcatactcctttacaccacactgtcgtcgaatggc

SEQ ID NO: 669 - DNA, 102 bp, synthetic construct
  source 1..102: mol_type = other DNA
ctcagcagcagccacaactccctcgcgcaacgaagaacaacctttcatgtgtttacatccacggctatcatactcctttacaccacactgtcgtcgaatggc

SEQ ID NO: 670 - DNA, 101 bp, synthetic construct
  source 1..101: mol_type = other DNA
ctcagcagcagccacaactccctcgcgcaactttgttaagactttcatgaatactctccacggctatcatactcctttacaccacactgtcgtcgaatggc

SEQ ID NO: 671 - DNA, 100 bp, synthetic construct
  source 1..100: mol_type = other DNA
ctcagcagcagccacaactccctcgcgcaactttgatcgtcctttgatgaatccatccacggctatcatactcctttacaccacactgtcgtcgaatggc

SEQ ID NO: 672 - DNA, 97 bp, synthetic construct
  source 1..97: mol_type = other DNA
ctcagcagcagccacaactccctcgcgcaacttttagtatttcatgaatacaaccacggctatcatactcctttacaccacactgtcgtcgaatggc

SEQ ID NO: 673 - DNA, 100 bp, synthetic construct
  source 1..100: mol_type = other DNA
ctcagcagcagccacaactccctcgcgcaactttgaaagtgtttcatgaatactttccacggctatcatactcctttacaccacactgtcgtcgaatggc

SEQ ID NO: 674 - DNA, 99 bp, synthetic construct
  source 1..99: mol_type = other DNA
ctcagcagcagccacaactccctcgcgcaactttgaaagactttcatgaatccatccacggctatcatactcctttacaccacactgtcgtcgaatggc

SEQ ID NO: 675 - DNA, 98 bp, synthetic construct
  source 1..98: mol_type = other DNA
ctcagcagcagccacaactccctcgcgcaacttcctaagggttcatccttacatccacggctatcatactcctttacaccacactgtcgtcgaatggc

SEQ ID NO: 676 - DNA, 100 bp, synthetic construct
  source 1..100: mol_type = other DNA
ctcagcagcagccacaactccctcgcgcaactttgaaagtccgttcatgcatagagccacggctacataattccttttggagtcactgtcgtcgaatggc

SEQ ID NO: 677 - DNA, 100 bp, synthetic construct
  source 1..100: mol_type = other DNA
ctcagcagcagccacaactccctcgcgcaacattgaaagtcctttcatgaatagtttcacggctatcatactcctttacaccacactgtcgtcgaatggc

SEQ ID NO: 678 - DNA, 100 bp, synthetic construct
  source 1..100: mol_type = other DNA
ctcagcagcagccacaactccctcgcgcaacttataacctcctttcatgaatagatccacggctatcatactcctttacaccacactgtcgtcgaatggc

SEQ ID NO: 679 - DNA, 98 bp, synthetic construct
  source 1..98: mol_type = other DNA
ctcagcagcagccacaactccctcgcgcaactttgatcgtcctttcatctacatctacggctatcatactcctttacaccacactgtcgtcgaatggc

SEQ ID NO: 680 - DNA, 98 bp, synthetic construct
  source 1..98: mol_type = other DNA
ctcagcagcagccacaactccctcgcgcaactttgaccctttcatgtctttacatccacggcaatcgccgactttacaccacactgtcgtcgaatggc

SEQ ID NO: 681 - DNA, 99 bp, synthetic construct
  source 1..99: mol_type = other DNA
ctcagcagcagccacaactccctcgcgcaactttgaaagtcatctcatgcatagaccacggctacataattccttttggagtcactgtcgtcgaatggc

SEQ ID NO: 682 - DNA, 104 bp, synthetic construct
  source 1..104: mol_type = other DNA
ctcagcagcagccacaactccctcgcgcaacgatgatgcagtcctttcatgaataaagatccacggctatcatactcctttacaccacactgtcgtcgaatggc

SEQ ID NO: 683 - DNA, 100 bp, synthetic construct
  source 1..100: mol_type = other DNA
ctcagcagcagccacaactccctcctctcgagttgaaagagctttcatgaatacaaccacggctacggatagagcttacaccacactgtcgtcgaatggc

SEQ ID NO: 684 - DNA, 102 bp, synthetic construct
  source 1..102: mol_type = other DNA
ctcagcagcagccacaactccctcgcgcaactttgatgacgtgggttcatctttacatccacggctatcatactcctttacaccacactgtcgtcgaatggc

SEQ ID NO: 685 - DNA, 99 bp, synthetic construct
  source 1..99: mol_type = other DNA
ctcagcagcagccacaactccctcgcgcaactttgaaagtatttcatgaatacagccacggctatcatactcctttacaccacactgtcgtcgaatggc

SEQ ID NO: 686 - DNA, 98 bp, synthetic construct
  source 1..98: mol_type = other DNA
ctcagcagcagccacaactccctcctatagagttgaaaggtttcatcaatacatctacggctaatcgccgactttacaccacactgtcgtcgaatggc

SEQ ID NO: 687 - DNA, 98 bp, synthetic construct
  source 1..98: mol_type = other DNA
ctcagcagcagccacaactccctcgcgcaactttgaaaggtttcatgaacccatccacggctatcatactcctttacaccacactgtcgtcgaatggc

SEQ ID NO: 688 - DNA, 98 bp, synthetic construct
  source 1..98: mol_type = other DNA
ctcagcagcagccacaactccctcgcgcaactttgaaaggtttcatgaatacagccacggctatcatactcctttacaccacactgtcgtcgaatggc

SEQ ID NO: 689 - DNA, 100 bp, synthetic construct
  source 1..100: mol_type = other DNA
ctcagcagcagccacaactccctcgcgcaacattgaaagtatttcatgaatacatagcacggctatcatactcctttacaccacactgtcgtcgaatggc

SEQ ID NO: 690 - DNA, 100 bp, synthetic construct
  source 1..100: mol_type = other DNA
ctcagcagcagccacaactccctcgcgcaactttgaaagtccagtcatgataacatccacggctatcatactcctttacaccacactgtcgtcgaatggc

SEQ ID NO: 691 - DNA, 99 bp, synthetic construct
  source 1..99: mol_type = other DNA
ctcagcagcagccacaactccctcctctcgagttgaaagtgtttcatgcttacatccacggctacggatagagcttacaccacactgtcgtcgaatggc

SEQ ID NO: 692 - DNA, 97 bp, synthetic construct
  source 1..97: mol_type = other DNA
ctcagcagcagccacaactccctcgcgcaactttgatgcctttcattataacatccacggcaatcgccgactttacaccacactgtcgtcgaatggc

SEQ ID NO: 693 - DNA, 98 bp, synthetic construct
  source 1..98: mol_type = other DNA
ctcagcagcagccacaactccctcgcgcaacttcaagcactttcatgactacatccacggctatcatactcctttacaccacactgtcgtcgaatggc

SEQ ID NO: 694 - DNA, 100 bp, synthetic construct
  source 1..100: mol_type = other DNA
ctcagcagcagccacaactccctcgcgcaacttattaagtcctttcatgaagccatctacggctatcatactcctttacaccacactgtcgtcgaatggc

SEQ ID NO: 695 - DNA, 99 bp, synthetic construct
  source 1..99: mol_type = other DNA
ctcagcagcagccacaactccctcgcgcaactttgaaagtcattcatgtatacaaccacggctatcatactcctttacaccacactgtcgtcgaatggc

SEQ ID NO: 696 - DNA, 99 bp, synthetic construct
  source 1..99: mol_type = other DNA
ctcagcagcagccacaactccctcctctcgagttgaaagtcctggcatgaatacaccacggctacggatagagcttacaccacactgtcgtcgaatggc

SEQ ID NO: 697 - DNA, 103 bp, synthetic construct
  source 1..103: mol_type = other DNA
ctcagcagcagccacaactccctcgcgcaactttaccaaaaggggtttcatctttacatccacggctatcatactcctttacaccacactgtcgtcgaatggc

SEQ ID NO: 698 - DNA, 101 bp, synthetic construct
  source 1..101: mol_type = other DNA
ctcagcagcagccacaactccctcgcgcaactttataaagtcctttggatgaatacaccacggctatcatactcctttacaccacactgtcgtcgaatggc

SEQ ID NO: 699 - DNA, 98 bp, synthetic construct
  source 1..98: mol_type = other DNA
ctcagcagcagccacaactccctcgcgcaactttgacagtattcatgtttacatccacggctatcatactcctttacaccacactgtcgtcgaatggc

SEQ ID NO: 700 - DNA, 99 bp, synthetic construct
  source 1..99: mol_type = other DNA
ctcagcagcagccacaactccctcgcgcaactttgaaagcaatttcatgcatagaccacggctacataattccttttggagtcactgtcgtcgaatggc

SEQ ID NO: 701 - DNA, 102 bp, synthetic construct
  source 1..102: mol_type = other DNA
ctcagcagcagccacaactccctcgcgcaacttttccaaagtcctttgatgaatacaagcacggctatcatactcctttacaccacactgtcgtcgaatggc

SEQ ID NO: 702 - DNA, 97 bp, synthetic construct
  source 1..97: mol_type = other DNA
ctcagcagcagccacaactccctcgcgcaactttgaaatatttcatctctacaccacggctatcatactcctttacaccacactgtcgtcgaatggc

SEQ ID NO: 703 - DNA, 103 bp, synthetic construct
  source 1..103: mol_type = other DNA
ctcagcagcagccacaactccctcgcgcaactttgaaactcttcctttcatgctgcacatccacggcaatcgccgactttacaccacactgtcgtcgaatggc

SEQ ID NO: 704 - DNA, 98 bp, synthetic construct
  source 1..98: mol_type = other DNA
ctcagcagcagccacaactccctcgcgcaactttgaaagatttcatgaatagatctacggctatcatactcctttacaccacactgtcgtcgaatggc

SEQ ID NO: 705 - DNA, 98 bp, synthetic construct
  source 1..98: mol_type = other DNA
ctcagcagcagccacaactccctcctatagagttgtgtcctttcatgtatacatctacggctaatcgccgactttacaccacactgtcgtcgaatggc

SEQ ID NO: 706 - DNA, 100 bp, synthetic construct
  source 1..100: mol_type = other DNA
ctcagcagcagccacaactccctcgcgcaacttcaggaaagatttcatgaatacatctacggctatcatactcctttacaccacactgtcgtcgaatggc

SEQ ID NO: 707 - DNA, 99 bp, synthetic construct
  source 1..99: mol_type = other DNA
ctcagcagcagccacaactccctcgcgcaactttgaaagtgtttcctgaatactgccacggctatcatactcctttacaccacactgtcgtcgaatggc

SEQ ID NO: 708 - DNA, 99 bp, synthetic construct
  source 1..99: mol_type = other DNA
ctcagcagcagccacaactccctcgcgcaactttgaaagtggcttcatgcatagaccacggctacataattccttttggagtcactgtcgtcgaatggc

SEQ ID NO: 709 - DNA, 99 bp, synthetic construct
  source 1..99: mol_type = other DNA
ctcagcagcagccacaactccctcctatagagttgaaagactttcatgtctacatctacggctaatcgccgactttacaccacactgtcgtcgaatggc

SEQ ID NO: 710 - DNA, 100 bp, synthetic construct
  source 1..100: mol_type = other DNA
ctcagcagcagccacaactccctcctatagagttgaccctcctttcatgaataccgctacggctaatcgccgactttacaccacactgtcgtcgaatggc

SEQ ID NO: 711 - DNA, 99 bp, synthetic construct
  source 1..99: mol_type = other DNA
ctcagcagcagccacaactccctcgcgcaactttgaaagtcgttcatgacgccatctacggctatcatactcctttacaccacactgtcgtcgaatggc

SEQ ID NO: 712 - DNA, 100 bp, synthetic construct
  source 1..100: mol_type = other DNA
ctcagcagcagccacaactccctcctctcgagttgaaagtgacttcatgagtacatccacggctacggatagagcttacaccacactgtcgtcgaatggc

SEQ ID NO: 713 - DNA, 99 bp, synthetic construct
  source 1..99: mol_type = other DNA
ctcagcagcagccacaactccctcgcgcaactttgaaagtgttcatgaataaaatctacggctatcatactcctttacaccacactgtcgtcgaatggc

SEQ ID NO: 714 - DNA, 101 bp, synthetic construct
  source 1..101: mol_type = other DNA
ctcagcagcagccacaactccctcctctcgagttgaaagggacctttcatgaatacaccacggctacggatagagcttacaccacactgtcgtcgaatggc

SEQ ID NO: 715 - DNA, 102 bp, synthetic construct
  source 1..102: mol_type = other DNA
ctcagcagcagccacaactccctcgcgcaactttgaacgtgtcctttcatgaatacaagcacggctatcatactcctttacaccacactgtcgtcgaatggc

SEQ ID NO: 716 - DNA, 100 bp, synthetic construct
  source 1..100: mol_type = other DNA
ctcagcagcagccacaactccctcctctcgagttgaaagtagattcatgattacatccacggctacggatagagcttacaccacactgtcgtcgaatggc

SEQ ID NO: 717 - DNA, 103 bp, synthetic construct
  source 1..103: mol_type = other DNA
ctcagcagcagccacaactccctcgcgcaacttaacgatcagtcctttcatgaatacatgcacggctatcatactcctttacaccacactgtcgtcgaatggc

SEQ ID NO: 718 - DNA, 96 bp, synthetic construct
  source 1..96: mol_type = other DNA
ctcagcagcagccacaactccctcgcgcaactttgaaccctttcacatacatctacggctatcatactcctttacaccacactgtcgtcgaatggc

SEQ ID NO: 719 - DNA, 99 bp, synthetic construct
  source 1..99: mol_type = other DNA
ctcagcagcagccacaactccctcgcgcaactttgaaagtccggtcatgcatagaccacggctacataattccttttggagtcactgtcgtcgaatggc

SEQ ID NO: 720 - DNA, 100 bp, synthetic construct
  source 1..100: mol_type = other DNA
ctcagcagcagccacaactccctcctgaatgattgaaagtccttccctgaatacatccacggcgtcatcattcctttacaccacactgtcgtcgaatggc

SEQ ID NO: 721 - DNA, 98 bp, synthetic construct
  source 1..98: mol_type = other DNA
ctcagcagcagccacaactccctcgcgcaactttgaaagtgttcatgcctaccgccacggctatcatactcctttacaccacactgtcgtcgaatggc

SEQ ID NO: 722 - DNA, 99 bp, synthetic construct
  source 1..99: mol_type = other DNA
ctcagcagcagccacaactccctcctctcgagttgaatcccctttcatgaatacaccacggctacggatagagcttacaccacactgtcgtcgaatggc

SEQ ID NO: 723 - DNA, 102 bp, synthetic construct
  source 1..102: mol_type = other DNA
ctcagcagcagccacaactccctcctctcgagttgaaagaaacctttcatgaataccaccacggctacggatagagcttacaccacactgtcgtcgaatggc

SEQ ID NO: 724 - DNA, 95 bp, synthetic construct
  source 1..95: mol_type = other DNA
cagcagccacaactcccgaaggagcctttgaaagtccttaattgaatacatccacggctatacttatcctttacaccacactgtcgtcgaatggt

SEQ ID NO: 725 - DNA, 95 bp, synthetic construct
  source 1..95: mol_type = other DNA
cagcagccacaactcccgaaggagcctttgaaagtccttaattgaatacatccacggctatacttatcctttgcatcacgctgttgttgagtggt

SEQ ID NO: 726 - DNA, 95 bp, synthetic construct
  source 1..95: mol_type = other DNA
cagcagccacaactcccgaaggagcctttgaaagtccttaattgagtatatccacggttatacttattctttgcatcacgctgttgttgagtggt

SEQ ID NO: 727 - DNA, 95 bp, synthetic construct
  source 1..95: mol_type = other DNA
cagcagccacaactctcgaaggagcctttgagagtccttaattgagtatatccacggttatacttattctttgcatcacgctgttgttgagtggt

SEQ ID NO: 728 - DNA, 95 bp, synthetic construct
  source 1..95: mol_type = other DNA
cagtagccacgactctcgaaggagcctttgagagtccttaattgagtatatccacggttatacttattctttgcatcacgctgttgttgagtggt

SEQ ID NO: 729 - DNA, 95 bp, synthetic construct
  source 1..95: mol_type = other DNA
cagcagccacaactcccgaaggagcctttgaaagtcctttattgaatacatccacggctaatgaaatcctttacaccacactgtcgtcgaatggc

SEQ ID NO: 730 - DNA, 100 bp, synthetic construct
  source 1..100: mol_type = other DNA
ctcagcagcagccacaactccgtggtaggcctttgaaagtcctttcatgaatacatctacggcattccttctcctttacaccacactgtcgtcgaatggc

SEQ ID NO: 731 - DNA, 100 bp, synthetic construct
  source 1..100: mol_type = other DNA
ctcagcagcggccacagctccatggaaggcttttggaagttcttttatgagtacgtctacggcatcactcctcctttgcatcacactgtcgttgagtggc

SEQ ID NO: 732 - DNA, 100 bp, synthetic construct
  source 1..100: mol_type = other DNA
ctcagcagcggccacagctccgtggaaggcttttggaagttcttttatgagtacgtctacggcatccctcctcctttgcatcacactgtcgttgagtggc

SEQ ID NO: 733 - DNA, 100 bp, synthetic construct
  source 1..100: mol_type = other DNA
ctcagtagcggccacagctccgtggaaggcttttggaagttcttttatgagtacgtctacggcatccctcctcctttgcatcacactgtcgttgagtggc

SEQ ID NO: 734 - DNA, 100 bp, synthetic construct
  source 1..100: mol_type = other DNA
ctcagtagcggccacagctccgtggaaggcttttggaagttcttttatgagtacgtctacggcattcctcctcctttgcatcacactgtcgttgagtggc

SEQ ID NO: 735 - DNA, 100 bp, synthetic construct
  source 1..100: mol_type = other DNA
ctcagtagcggccacagctccgtggaaggcttttggaagttcttttatgagtacgtctacggcattcctcctcctttgcatcgcactgtcgttgagtggc

SEQ ID NO: 736 - DNA, 100 bp, synthetic construct
  source 1..100: mol_type = other DNA
ctcagtagcggccacagctccgtggaaggcttttggaagttcttttatgagtacgtctacggcattccttctcctttgcatcgcactgtcgttgagtggc

SEQ ID NO: 737 - DNA, 100 bp, synthetic construct
  source 1..100: mol_type = other DNA
ctcagtagcggccacagctccgtggtaggcttttggaagttcttttatgagtacgtctacggcattccttctcctttgcatcgcactgtcgttgagtggc

SEQ ID NO: 738 - DNA, 100 bp, synthetic construct
  source 1..100: mol_type = other DNA
cttagtagcggccacagctccgtggtaggcttttggaagttcttttatgagtacgtctacggcattccttctcctttgcatcgcactgtcgttgagtggc

SEQ ID NO: 739 - DNA, 100 bp, synthetic construct
  source 1..100: mol_type = other DNA
ctcagcagcagccacaactccgtggacggcctttgaaagtcctttcatgaatagttccacggctacggtcatcctttacaccacactgtcgtcgaatggc

SEQ ID NO: 740 - DNA, 100 bp, synthetic construct
  source 1..100: mol_type = other DNA
ctcagcagcagtcacgactccgtggtaggcctttgagagtcctttcgtgaatagttccacggctacggtcatcctttacatcacactgtcgtcggatggc

SEQ ID NO: 741 - DNA, 100 bp, synthetic construct
  source 1..100: mol_type = other DNA
ctcagcagcagtcacgactccgtggacggcctttgagagtcctttcgtgaatagttccacggctacggtcatcctttacatcacactgtcgtcggatggc

SEQ ID NO: 742 - DNA, 100 bp, synthetic construct
  source 1..100: mol_type = other DNA
ctcagcagcagtcacaactccgtggacggcctttgagagttctttcgtgaatagttccacggctacggtcatcctttacatcacactgttgtcggatggc

SEQ ID NO: 743 - DNA, 100 bp, synthetic construct
  source 1..100: mol_type = other DNA
ctcagcagcagtcacgactccgtggacggcctttgagagttctttcgtgaatagttccacggctacggtcatcctttacatcacactgttgtcggatggc

SEQ ID NO: 744 - DNA, 100 bp, synthetic construct
  source 1..100: mol_type = other DNA
ctcagcagcagtcacgactccgtggacggtctttgagagtcctttcgtgaatagttccacggctacggtcatcctttacatcacgctgttgtcggatggc

SEQ ID NO: 745 - DNA, 100 bp, synthetic construct
  source 1..100: mol_type = other DNA
ctcagcagcagtcacgactccgtggacggtctttgagagttctttcgtgaatagttccacggctacggtcatcctttacatcacgctgttgtcggatggc

SEQ ID NO: 746 - DNA, 100 bp, synthetic construct
  source 1..100: mol_type = other DNA
ctcagcggcagtcacgactccgtggacggtctttgagagtcctttcgtgaatagttccacggctacggtcatcctttacatcacgctgttgtcggatggt

SEQ ID NO: 747 - DNA, 100 bp, synthetic construct
  source 1..100: mol_type = other DNA
ctcagcggcagtcacgactccgtggacggtctttgagagttctttcgtgaatagttccacggctacggtcatcctttacatcacgctgttgtcggatggt

SEQ ID NO: 748 - DNA, 100 bp, synthetic construct
  source 1..100: mol_type = other DNA
ctcagcagcagccacaactccctcgcgcaactttgaaagtcctttcatgcatagatccacggctacataattccttttggagtcactgtcgtcgaatggc

SEQ ID NO: 749 - DNA, 100 bp, synthetic construct
  source 1..100: mol_type = other DNA
ctcagcagcagccacaactccctcctatagagttgaaagtcctttcatgaatacatctacggctaatcgccgactttacaccacactgtcgtcgaatggc

SEQ ID NO: 750 - DNA, 90 bp, synthetic construct
  source 1..90: mol_type = other DNA
cagcagccacaactccctcaaaccgctttgaaagtcctttcatgtatagatccacggctatacaattccttttgtggtcgctgttgttgg

SEQ ID NO: 751 - DNA, 90 bp, synthetic construct
  source 1..90: mol_type = other DNA
cagcagccacagctctctcacaccgctttggaagttctttcgtgcatggatccacggctatacaatttcttttgtggtcgctgttgttga

SEQ ID NO: 752 - DNA, 90 bp, synthetic construct
  source 1..90: mol_type = other DNA
cagcagccacagctctctcaaaccgctttggaagttctttcgtgcatggatccacggctatacaatttcttttgtggtcgctgttgttga

SEQ ID NO: 753 - DNA, 90 bp, synthetic construct
  source 1..90: mol_type = other DNA
cagcagccacagctctctcaaaccgctttggaggttctttcgtgcatggatccacggctatacaatttcttttgtggtcgctgttgttga

SEQ ID NO: 754 - DNA, 90 bp, synthetic construct
  source 1..90: mol_type = other DNA
cagtagccacagctctctcaaaccgctttggaggttctttcgtgcatggatccacggctatacaatttcttttgtggtcgctgttgttga

SEQ ID NO: 755 - DNA, 90 bp, synthetic construct
  source 1..90: mol_type = other DNA
cagtagccacagctctctcaaaccgctttggaggttctttcgtgcatggatccacggttatacaatttcttttgtggtcgctgttgttga

SEQ ID NO: 756 - DNA, 90 bp, synthetic construct
  source 1..90: mol_type = other DNA
cagtagccacagctctctcaaaccgctttggaggttctttcgtgcatggattcacggttatacaatttcttttgtggtcgctgttgttga

SEQ ID NO: 757 - DNA, 90 bp, synthetic construct
  source 1..90: mol_type = other DNA
cagtagccacagctctctcaaaccgctttggaggttctttcgtgtatggattcacggttatacaatttcttttgtggtcgctgttgttga

SEQ ID NO: 758 - DNA, 90 bp, synthetic construct
  source 1..90: mol_type = other DNA
cagtagccacagctctctcaaaccgctttggaggttctttcgtgtatggattcacggttatacaatttcttttgtggtcgctgttgttgg

SEQ ID NO: 759 - DNA, 90 bp, synthetic construct
  source 1..90: mol_type = other DNA
cagtagctacagctctctcaaaccgctttggaggttctttcgtgtatggattcacggttatacaatttcttttgtggtcgctgttgttgg

SEQ ID NO: 760 - DNA, 86 bp, synthetic construct
  source 1..86: mol_type = other DNA
cagcagccacaactccctcctacaaacttgaaagtcctttcatgaatacaaccacggctaattgacggagattacaccacactgtt

SEQ ID NO: 761 - DNA, 86 bp, synthetic construct
  source 1..86: mol_type = other DNA
cagcagccacgactcccttctccaagattgagagttcttttatgagtacacccacggctagttgatgaggattgcgccacgctgtt

SEQ ID NO: 762 - DNA, 86 bp, synthetic construct
  source 1..86: mol_type = other DNA
cagcagccacgactcccttctccaagattgagagttcttttatgagtacacccacggctagttgatgaggattgcgctacgctgtt

SEQ ID NO: 763 - DNA, 86 bp, synthetic construct
  source 1..86: mol_type = other DNA
cagcagtcacgactcccttctccaagattgagagttcttttatgagtacacccacggctagttgatgaggattgcgctacgctgtt

SEQ ID NO: 764 - DNA, 86 bp, synthetic construct
  source 1..86: mol_type = other DNA
cagcagtcacgactcccttctccaagattgagagttcttttataagtacacccacggctagttgatgaggattgcgctacgctgtt

SEQ ID NO: 765 - DNA, 86 bp, synthetic construct
  source 1..86: mol_type = other DNA
cagcagtcacgactctcttctccaagattgagagttcttttataagtacacccacggctagttgatgaggattgcgctacgctgtt

SEQ ID NO: 766 - DNA, 86 bp, synthetic construct
  source 1..86: mol_type = other DNA
cagcagtcacgactctcttctccaagattgagagttcttttataagtacacccacggctagttgataaggattgcgctacgctgtt

SEQ ID NO: 767 - DNA, 86 bp, synthetic construct
  source 1..86: mol_type = other DNA
cagtagtcacgactctcttctccaagattgagagttcttttataagtacacccacggctagttgataaggattgcgctacgctgtt

SEQ ID NO: 768 - DNA, 98 bp, synthetic construct
  source 1..98: mol_type = other DNA
ctcagcggcagtcacagctccttcctacatgattggaagtctttttatggatacgtccacggcatgatatgggttgcatcacgctgttgtcggatggc

SEQ ID NO: 769 - DNA, 98 bp, synthetic construct
  source 1..98: mol_type = other DNA
ctcagcggcagtcacagctccttcctacatgattggaagtctttttatggatacgtccacggcatgatatgggttgcatcacgctgttgtcggatggt

SEQ ID NO: 770 - DNA, 98 bp, synthetic construct
  source 1..98: mol_type = other DNA
ctcagcggcagtcacagctccttcctacatgattggaagtctttttatggatacgtccacggcatgatatggattgcatcacgctgttgtcggatggt

SEQ ID NO: 771 - DNA, 98 bp, synthetic construct
  source 1..98: mol_type = other DNA
ctcagcggcagtcacagctccttcctacatgattggaagtctttttatggatacgtccacggcatgatatggattgcatcacgctgttgttggatggt

SEQ ID NO: 772 - DNA, 98 bp, synthetic construct
  source 1..98: mol_type = other DNA
ctcagcggcagtcacagctccttcctacatgattggaagtctttttatggatacgtccacggcatgatatggattgcatcgcgctgttgttggatggt

SEQ ID NO: 773 - DNA, 98 bp, synthetic construct
  source 1..98: mol_type = other DNA
ctcagcggcagtcacagctccttcctacatgattggaagtctttttatggatacgtccacggcatgataaggattgcatcgcgctgttgttggatggt

SEQ ID NO: 774 - DNA, 98 bp, synthetic construct
  source 1..98: mol_type = other DNA
ctcagcggcagtcacagctccttcctacatgattggaggtctttttatggatacgtccacggcatgataaggattgcatcgcgctgttgttggatggt

SEQ ID NO: 775 - DNA, 98 bp, synthetic construct
  source 1..98: mol_type = other DNA
cttagcggcagtcacagctccttcctacatgattggaggtctttttatggatacgtccacggcatgataaggattgcatcgcgctgttgttggatggt

SEQ ID NO: 776 - DNA, 98 bp, synthetic construct
  source 1..98: mol_type = other DNA
cttagcggcagtcacagctccttcctacatgattggaggtctttttatggatacgtccacggcatgataaggattgcatcgcgctgttgttgggtggt

SEQ ID NO: 777 - RNA, 100 nt, synthetic construct
  source 1..100: mol_type = other RNA
ctcagcagcagccacaactccgtgcggggcctttgaaagtcctttcatgaatacatctacggctatactggtcctttacaccacactgtcgtcgaatggt

SEQ ID NO: 778 - RNA, 100 nt, synthetic construct
  source 1..100: mol_type = other RNA
ctcagcagcagccacaactccgtgcggggcctttgaaagtcctttcgtgagtacgtctacggctatactggttctttgcatcgcgctgttgtcgagtggt

SEQ ID NO: 779 - RNA, 100 nt, synthetic construct
  source 1..100: mol_type = other RNA
ctcagcagcagccacaactccgtgcggggtctttgaaagttctttcgtgagtacgtctacggctatactggttctttgcatcgcgctgttgtcgagtggt

SEQ ID NO: 780 - RNA, 100 nt, synthetic construct
  source 1..100: mol_type = other RNA
ctcagcagcagccacgacttcgtgcggggtctttgaaagttctttcgtgagtacgtctacggctatactggttctttgcatcgcgctgttgtcgagtggt

SEQ ID NO: 781 - RNA, 100 nt, synthetic construct
  source 1..100: mol_type = other RNA
ctcagcagcggctacgacttcgtgcggggtctttgaaagttctttcgtgagtacgtctacggctatactggttctttgcatcgcgctgttgtcgagtggt

SEQ ID NO: 782 - RNA, 100 nt, synthetic construct
  source 1..100: mol_type = other RNA
ctcggtagcggctacgacttcgtgcggggtctttgaaagttctttcgtgagtacgtctacggctatactggttctttgcatcgcgctgttgtcgagtggt

SEQ ID NO: 783 - RNA, 100 nt, synthetic construct
  source 1..100: mol_type = other RNA
ctcagcagcagccacaactccgtgcggggcctttgaaagtcctttcatgaatacatctacggctaatgaggtcctttacaccacactgtcgtcgaatggt

SEQ ID NO: 784 - RNA, 100 nt, synthetic construct
  source 1..100: mol_type = other RNA
ctcagcagcagccacaactccctccttgataggcgaaagtcctttcatgaataactccacggctatcatactcctttacaccacactgtcgtcgaatggc

SEQ ID NO: 785 - DNA, 100 bp, synthetic construct
  source 1..100: mol_type = other DNA
ctcagcagcagccacaactccgtgcggggcctttgaaagtcctttcatgaatacatctacggctatactggtcctttacaccacactgtcgtcgaatggt

SEQ ID NO: 786 - DNA, 100 bp, synthetic construct
  source 1..100: mol_type = other DNA
ctcagcagcagccacaactccgtgcggggcctttgaaagtcctttcgtgagtacgtctacggctatactggttctttgcatcgcgctgttgtcgagtggt

SEQ ID NO: 787 - DNA, 100 bp, synthetic construct
  source 1..100: mol_type = other DNA
ctcagcagcagccacaactccgtgcggggtctttgaaagttctttcgtgagtacgtctacggctatactggttctttgcatcgcgctgttgtcgagtggt

SEQ ID NO: 788 - DNA, 100 bp, synthetic construct
  source 1..100: mol_type = other DNA
ctcagcagcagccacgacttcgtgcggggtctttgaaagttctttcgtgagtacgtctacggctatactggttctttgcatcgcgctgttgtcgagtggt

SEQ ID NO: 789 - DNA, 100 bp, synthetic construct
  source 1..100: mol_type = other DNA
ctcagcagcggctacgacttcgtgcggggtctttgaaagttctttcgtgagtacgtctacggctatactggttctttgcatcgcgctgttgtcgagtggt

SEQ ID NO: 790 - DNA, 100 bp, synthetic construct
  source 1..100: mol_type = other DNA
ctcggtagcggctacgacttcgtgcggggtctttgaaagttctttcgtgagtacgtctacggctatactggttctttgcatcgcgctgttgtcgagtggt

SEQ ID NO: 791 - DNA, 100 bp, synthetic construct
  source 1..100: mol_type = other DNA
ctcagcagcagccacaactccgtgcggggcctttgaaagtcctttcatgaatacatctacggctaatgaggtcctttacaccacactgtcgtcgaatggt

SEQ ID NO: 792 - DNA, 100 bp, synthetic construct
  source 1..100: mol_type = other DNA
ctcagcagcagccacaactccctccttgataggcgaaagtcctttcatgaataactccacggctatcatactcctttacaccacactgtcgtcgaatggc

SEQ ID NO: 793 - DNA, 10 bp, synthetic construct
  source 1..10: mol_type = other DNA
aaaaaggggg

SEQ ID NO: 794 - DNA, 10 bp, synthetic construct
  source 1..10: mol_type = other DNA
aaaaaaaaaa

SEQ ID NO: 795 - DNA, 20 bp, synthetic construct
  source 1..20: mol_type = other DNA
cccccggggggggggccccc

SEQ ID NO: 796 - DNA, 10 bp, synthetic construct
  source 1..10: mol_type = other DNA
gggggggggg

SEQ ID NO: 797 - protein, 10 aa, synthetic construct
FFFFFYYYYY

SEQ ID NO: 798 - protein, 10 aa, synthetic construct
YYYYYYYYYY

SEQ ID NO: 799 - protein, 20 aa, synthetic construct
LLLLLFFFFFYYYYYLLLLL

SEQ ID NO: 800 - DNA, 105 bp, synthetic construct
  source 1..105: mol_type = other DNA
  misc_difference 1: note = a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 801 - DNA, 105 bp, synthetic construct
  source 1..105: mol_type = other DNA
  misc_difference 105: note = a, c, t, g, unknown or other
agcagccacaactccgggaacccctttgaaagtcctttcccgaatacatccacggctaaacttctcctttacaccacactgtcgtcgattaggccactcccagtn

SEQ ID NO: 802 - RNA, 24 nt, synthetic construct
  source 1..24: mol_type = other RNA
  modified_base 8: mod_base = OTHER; note = Inosine
  modified_base 21: mod_base = OTHER; note = Inosine
gtgcacantggtctacgtgtngat

SEQ ID NO: 803 - RNA, 24 nt, synthetic construct
  source 1..24: mol_type = other RNA
  modified_base 21: mod_base = OTHER; note = Inosine
gtgcacaatggtctacgtgtngat

SEQ ID NO: 804 - DNA, 38 bp, synthetic construct
  source 1..38: mol_type = other DNA
agtgtggtgtaaaggaattcattagccatggatgtatt

SEQ ID NO: 805 - DNA, 42 bp, synthetic construct
  source 1..42: mol_type = other DNA
atgaaaggactttcaaaggccaaggagggagttgtggctgct

SEQ ID NO: 806 - DNA, 38 bp, synthetic construct
  source 1..38: mol_type = other DNA
aatacatctacggctacatatctcctttacaccacact

SEQ ID NO: 807 - DNA, 42 bp, synthetic construct
  source 1..42: mol_type = other DNA
agcagccacaactccaatgaaggcctttgaaagtcctttcat

SEQ ID NO: 808 - DNA, 22 bp, synthetic construct
  source 1..22: mol_type = other DNA
aaaggaattcattagccatgga

SEQ ID NO: 809 - DNA, 22 bp, synthetic construct
  source 1..22: mol_type = other DNA
tctacggcaccatcattccttt

SEQ ID NO: 810 - DNA, 23 bp, synthetic construct
  source 1..23: mol_type = other DNA
aaaggaattcattagccatggat

SEQ ID NO: 811 - DNA, 23 bp, synthetic construct
  source 1..23: mol_type = other DNA
atctacggcaccatccatccttt

SEQ ID NO: 812 - DNA, 24 bp, synthetic construct
  source 1..24: mol_type = other DNA
taaaggaattcattagccatggat

SEQ ID NO: 813 - DNA, 23 bp, synthetic construct
  source 1..23: mol_type = other DNA
atctacggcaataccgcatttta